(19) United States
(12) Patent Application Publication
(10) Pub. No.: US 2023/0096028 A1
(43) Pub. Date: Mar. 30, 2023

(54) BICYCLIC HETEROCYCLYL COMPOUNDS AND USES THEREOF

(71) Applicant: Revolution Medicines, Inc., Redwood City, CA (US)

(72) Inventors: James Joseph CREGG, Redwood City, CA (US); Andreas BUCKL, Redwood City, CA (US); Naing AAY, Redwood City, CA (US); Arlyn A. TAMBO-ONG, Redwood City, CA (US); Elena S. KOLTUN, Redwood City, CA (US); Adrian Liam GILL, Redwood City, CA (US); Severin THOMPSON, Redwood City, CA (US); Micah J. Gliedt, Redwood City, CA (US)

(21) Appl. No.: 17/310,932

(22) PCT Filed: Mar. 2, 2020
(86) PCT No.: PCT/US2020/020609

§ 371 Date: Aug. 31, 2021

Related U.S. Application Data
(60) Provisional application No. 62/812,839, filed on Mar. 1, 2019.
(60) Provisional application No. 62/949,785, filed on Dec. 18, 2019.

(51) Int. Cl. C07D 471/04; C07D 519/00; C07D 239/94; C07D 491/107; C07D 487/04; A61P 35/00; A61P 11/00

(52) U.S. Cl. CPC C07D 471/04; C07D 519/00; C07D 239/94; C07D 491/107; C07D 487/04; A61P 35/00; A61P 11/00

(57) ABSTRACT

The present disclosure is directed to modulators of SOS1 and their use in the treatment of disease. Also disclosed are Q pharmaceutical compositions comprising the same.

### CROSS REFERENCE TO RELATED APPLICATION(S)

The present application claims the benefit of priority to U.S. provisional application Ser. No. 62/812,839, filed Mar. 1, 2019, the disclosure of which is hereby incorporated by reference as if set forth in its entirety. The present application claims the benefit of priority to U.S. provisional application Ser. No. 62/949,785, filed Dec. 18, 2019, the disclosure of which is hereby incorporated by reference as if set forth in its entirety.

### FIELD OF THE DISCLOSURE

The present disclosure relates to inhibitors of SOS1 useful in the treatment of diseases or disorders. Specifically, the present disclosure is concerned with compounds and compositions inhibiting SOS1, methods of treating diseases associated with SOS1, and methods of synthesizing these compounds.

### BACKGROUND OF THE DISCLOSURE

RAS-family proteins including KRAS (V-Ki-ras2 Kirsten rat sarcoma viral oncogene homolog), NRAS (neuroblastoma RAS viral oncogene homolog) and HRAS (Harvey murine sarcoma virus oncogene) and any mutants thereof are small GTPases that exist in cells in either GTP-bound or GDP-bound states (McCormick et al., J. Mol. Med. (Berl)., 2016, 94(3):253-8; Nimnual et al., Sci. STKE., 2002, 2002(145):pl36). The RAS-family proteins have a weak intrinsic GTPase activity and slow nucleotide exchange rates (Hunter et al., Mol. Cancer Res., 2015, 13(9): 1325-35). Binding of GTPase activating proteins (GAPs) such as NF1 increases the GTPase activity of RAS-family proteins. The binding of guanine nucleotide exchange factors (GEFs) such as SOS1 (Son of Sevenless 1) promote release of GDP from RAS-family proteins, enabling GTP binding (Chardin et al., Science, 1993, 260(5112):1338-43). When in the GTP-bound state, RAS-family proteins are active and engage effector proteins including RAF and phosphoinositide 3-kinase (PI3K) to promote the RAF/mitogen or extracellular signal-regulated kinases (MEK/ERK). Published data indicate a critical involvement of SOS1 in mutant KRAS activation and oncogenic signaling in cancer (Jeng et al., Nat. Commun., 2012, 3:1168). Depleting SOS1 levels decreased the proliferation rate and survival of tumor cells carrying a KRAS mutation whereas no effect was observed in KRAS wild type cell lines. The effect of loss of SOS1 could not be rescued by introduction of a catalytic site mutated SOS1, demonstrating the essential role of SOS1 GEF activity in KRAS mutant cancer cells.

SOS1 is critically involved in the activation of RAS-family protein signaling in cancer via mechanisms other than mutations in RAS-family proteins. SOS1 interacts with the adaptor protein Grb2 and the resulting SOS1/Grb2 complex binds to activated/phosphorylated Receptor Tyrosine Kinases (e.g., EGFR, ErbB2, ErbB3, ErbB4, PDGFR-A/B, FGFR1/2/3, IGF1 R, INSR, ALK, ROS, TrkA, TrkB, TrkC, RET, c-MET, VEGFR1/2/3, AXL) (Pierre et al., Biochem. Pharmacol., 2011, 82(9): 1049-56). SOS1 is also recruited to other phosphorylated cell surface receptors such as the T cell Receptor (TCR), B cell Receptor (BCR) and monocyte colony-stimulating factor receptor (Salojin et al., J. Biol. Chem. 2000, 275(8):5966-75). This localization of SOS1 to the plasma membrane, proximal to RAS-family proteins, enables SOS1 to promote RAS-family protein activation. SOS1-activation of RAS-family proteins can also be mediated by the interaction of SOS1/Grb2 with the BCR-ABL oncoprotein commonly found in chronic myelogenous leukemia (Kardinal et al., 2001, Blood, 98:1773-81; Sini et al., Nat. Cell Biol., 2004, 6(3):268-74). Furthermore, alterations in SOS1 have been implicated in cancer. SOS1 mutations are found in embryonal rhabdomyosarcomas, Sertoli cell testis tumors, granular cell tumors of the skin (Denayer et al., Genes Chromosomes Cancer, 2010, 49(3):242-52) and lung adenocarcinoma (Cancer Genome Atlas Research Network., Nature, 2014, 511 (751 1):543-50). Meanwhile over-expression of SOS1 has been described in bladder cancer (Watanabe et al., IUBMB Life, 2000, 49(4):317-20) and prostate cancer (Timofeeva et al., Int. J. Oncol., 2009; 35(4):751-60). In addition to cancer, hereditary SOS1 mutations are implicated in the pathogenesis of RASopathies like e.g., Noonan syndrome (NS), cardio-facio-cutaneous syndrome (CFC) and hereditary gingival fibromatosis type 1 (Pierre et al., Biochem. Pharmacol., 2011, 82(9):1049-56).

SOS1 is also a GEF for the activation of the GTPases RAC1 (Ras-related C3 botulinum toxin substrate 1) (Innocenti et al., J. Cell Biol., 2002, 156(1):125-36). RAC1, like RAS-family proteins, is implicated in the pathogenesis of a variety of human cancers and other diseases (Bid et al., Mol. Cancer Ther. 2013, 12(10):1925-34).

Son of Sevenless 2 (SOS2), a homolog of SOS1 in mammalian cells, also acts as a GEF for the activation of RAS-family proteins (Pierre et al., Biochem. Pharmacol., 2011, 82(9): 1049-56; Buday et al., Biochim. Biophys. Acta., 2008, 1786(2):178-87). Published data from mouse knockout models suggests a redundant role for SOS1 and SOS2 in homeostasis in the adult mouse. Whilst germline knockout of SOS1 in mice results in lethality during mid-embryonic gestation (Qian et al., EMBO J., 2000, 19(4):642-54), systemic conditional SOS1 knockout adult mice are viable (Baltanas et al., Mol. Cell. Biol., 2013, 33(22):4562-78). SOS2 gene targeting did not result in any overt phenotype in mice (Esteban et al., Mol. Cell. Biol., 2000, 20(17):6410-3). In contrast, double SOS1 and SOS2 knockout leads to rapid lethality in adult mice (Baltanas et al., Mol. Cell. Biol., 2013, 33(22):4562-78). These published data suggest that selective targeting of individual SOS isoforms (e.g., selective SOS1 targeting) may be adequately tolerated to achieve a therapeutic index between SOS1/RAS-family protein driven cancers (or other SOS1/RAS-family protein pathologies) and normal cells and tissues.

Selective pharmacological inhibition of the binding of the catalytic site of SOS1 to RAS-family proteins is expected to prevent SOS1-mediated activation of RAS-family proteins to the GTP-bound form. Such SOS1 inhibitor compounds are be expected to consequently inhibit signaling in cells downstream of RAS-family proteins (e.g., ERK phosphorylation). In cancer cells associated with dependence on RAS-family proteins (e.g., KRAS mutant cancer cell lines), SOS1 inhibitor compounds are be expected to deliver anti-cancer efficacy (e.g., inhibition of proliferation, survival, metastasis, etc). High potency towards inhibition of SOS1:RAS-family protein binding (nanomolar level IC₅₀ values) and ERK phosphorylation in cells (nanomolar level IC₅₀ values) are desirable characteristics for a SOS1 inhibitor compound. Furthermore, a desirable characteristic of a SOS1 inhibitor compound would be the selective inhibition of SOS1 over SOS2. This conclusion is based on the viable phenotype of SOS1 knockout mice and lethality of SOS1/SOS2 double knockout mice, as described above.

These characteristics have not been achieved in previously described SOS1 inhibitor compounds. In the last decades, the RAS family proteins-SOS1 protein interaction has gained increasing recognition. Several efforts to identify and optimize binders, which target either the effector binding site of RAS or the catalytic binding site of SOS1 (for a selected review see: Lu et al., Chem Med Chem. 2016, 11(8):814-21), have been made with limited success.

Recently, small activating molecules have been identified, which bind to a lipophilic pocket of SOS1 in close proximity to the RAS binding site (Burns et al., Proc. Natl. Acad. Sci. 2014, 111(9):3401-6). However, binding of these molecules seems to lead to increased nucleotide exchange and thereby activation of RAS instead of deactivation.

In an effort to stabilize the protein-protein-interaction of RAS-family proteins with SOS1 and to prevent reloading of RAS-family proteins with GTP, several different fragments were subsequently identified (Winter et al., J. Med. Chem. 2015, 58(5):2265-74). However, reversible binding of fragments to SOS1 did not translate into a measurable effect on the nucleotide exchange and only a weak effect was observed for fragments covalently bound to RAS.

Also recently, studies have been conducted to combine rational design and screening platforms to identify small molecule inhibitors of SOS1 (Evelyn et al., Chem. Biol. 2014, 21 (12):1618-28; Evelyn et al., J. Biol. Chem. 2015, 290(20):12879-98; Zheng et al., WO 2016/077793), i.e. compounds which bind to SOS1 and inhibit protein-protein interaction with RAS-family proteins. Although compounds with a slight inhibitory effect on SOS1 have been identified, the effects on guanine nucleotide exchange and cellular signal transduction modulation (e.g., ERK phosphorylation) are weak.

### BRIEF SUMMARY

The present disclosure relates to compounds capable of inhibiting the activity of SOS1. The present disclosure further provides a process for the preparation of compounds, pharmaceutical preparations comprising such compounds and methods of using such compounds and compositions in the management of diseases or disorders associated with the aberrant activity of SOS1.

One aspect of the present disclosure relates to compounds of Formula (I):

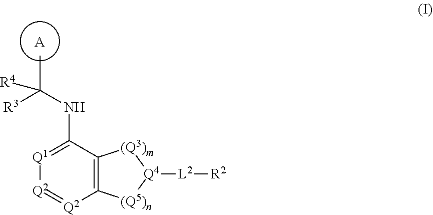

or a pharmaceutically acceptable salt, solvate, isomer, prodrug, or tautomer thereof, wherein:

Q¹ is CH or N;

Q⁴ is CH, C, or N;

each Q² is independently C—R¹ or N, wherein one Q² is N and the other Q² is C—R¹;

each Q³ and Q⁵ are independently C(R^{QC})₂, NR^{QN}, CO, O, S, or SO₂, wherein each R^{QC} is independently H, F, Cl, Br, or 6-10 membered aryl, and wherein each R^{QN} is independently H, C₁₋₆ alkyl, or 6-10 membered aryl;

wherein at least one of Q¹, Q², Q³, Q⁴, and Q⁵ is N, NR^{QN}, O, or SO₂;

m is 0, 1, 2, or 3;

n is 0, 1, 2, or 3;

wherein when m is 0, then n is not 0;

R¹ is selected from the group consisting of H, C₁₋₆ alkyl, halogen, —CONHR^{1a}, —NHR^{1a}, —OR^{1a}, cyclopropyl, azetidinyl, and —CN; wherein each C₁₋₆ alkyl and azetidinyl is optionally substituted with halogen, R^{1a}, —NHR^{1a}, or —OR^{1a}; wherein R^{1a} is H, C₁₋₆ alkyl, cyclopropyl, 3-6 membered heterocyclyl, or C1.6 haloalkyl;

L² is selected from the group consisting of a bond, —C(O)—, —C(O)O—, —C(O)NH(CH₂)ₒ—, —S(O)₂—,

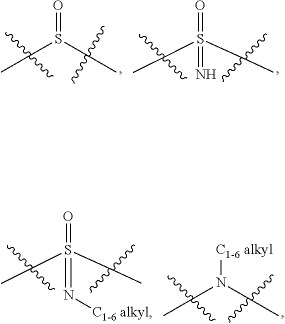

C(O)(CH₂)ₚ—, —(CH₂)ₚ—, and —O—; wherein o is 0, 1, or 2; and wherein p is a number from 1 to 6;

R² is selected from the group consisting of H, C₁₋₆ alkyl, —NR^{2b}R^{2c}, —OR^{2a}, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl are independently optionally substituted with C₁₋₆ alkyl, C₁₋₆ haloalkyl, C₁₋₆ hydroxyalkyl, C₁₋₆ methoxyalkyl, —OH, —OR^{2a}, oxo, ═N, halogen, —C(O)R^{2a}, —C(O)OR^{2a}, —C(O)NR^{2b}R^{2c}, —SO₂R^{2a}, —CN, —NR^{2b}R^{2c}, 3-6 membered cycloalkyl, 3-7 membered heterocyclyl, 6-10 membered aryl, or 5-10 membered heteroaryl;
wherein R^{2a} is H, C₁₋₆ alkyl, C₁₋₆ haloalkyl, 3-7 membered heterocyclyl, or —(CH₂)ᵣOCH₃, wherein r is 1, 2, or 3;
wherein R^{2b} is H or C₁₋₆ alkyl;
wherein R^{2c} is H or C₁₋₆ alkyl;

R³ and R⁴ are independently H or C₁₋₆ alkyl optionally substituted with halo or —OH; wherein at least one of R³ and R⁴ is H or wherein R³ and R⁴ together with the atom to which they are attached combine to form a 3-6 membered cycloalkyl; and

A is an optionally substituted 6-membered aryl or an optionally substituted 5-6 membered heteroaryl;

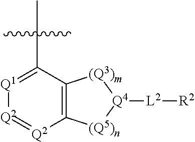

with the proviso that when

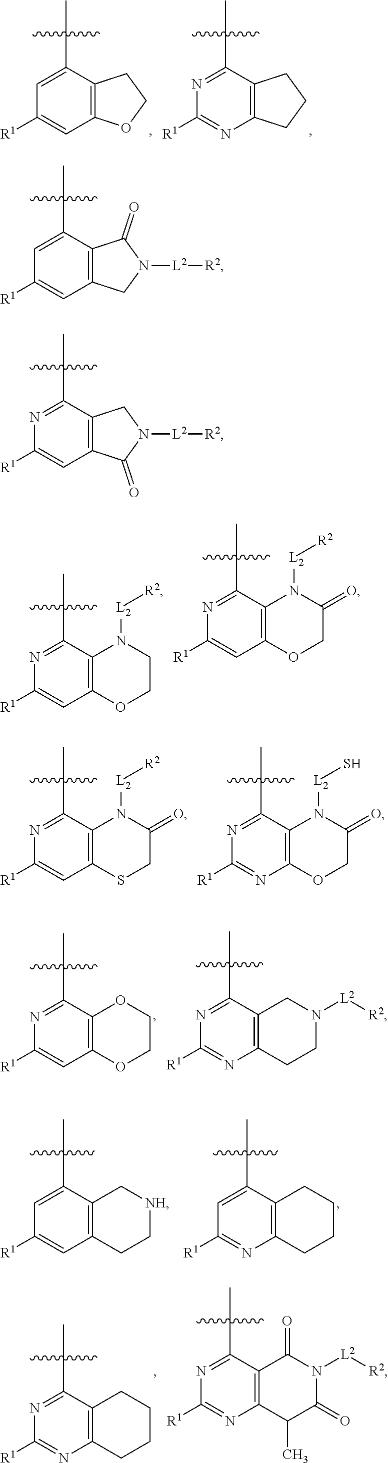

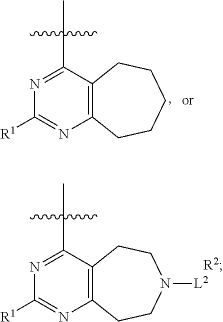

then R¹ is not H.

One aspect of the present disclosure relates to compounds of Formula (I-a):

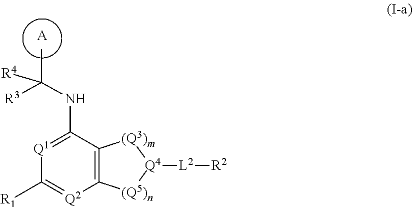

or a pharmaceutically acceptable salt, solvate, isomer, prodrug, or tautomer thereof, wherein:

Q¹, Q³, Q⁴, Q⁵, m, n and A are as defined in Formula (I);

Q² is CH or N;

wherein at least one of Q¹, Q², Q³, Q⁴, and Q⁵ is N, NR^{QN}, O, or SO₂;

R¹ is selected from the group consisting of H, halogen, C₁₋₆ alkyl, cyclopropyl, —CN, and —OR^{1a}; wherein R^{1a} is H or C₁₋₆ alkyl;

L² is selected from the group consisting of a bond, —C(O)—, —C(O)O—, —C(O)NH(CH₂)ₒ—, —S(O)₂—, —C(O)(CH₂)ₚ—, —(CH₂)ₚ—, or —O—; wherein o is 0, 1, or 2; and wherein p is a number from 1 to 6;

R² is selected from the group consisting of H, —(CH₂)_{q}CH₃, 3-14 membered cycloalkyl, 3-14 membered cycloalkenyl, 3-14 membered heterocyclyl, 6-10 membered aryl, and 5-10 membered heteroaryl; wherein q is a number from 1 to 5; wherein each 3-14 membered cycloalkyl, 3-14 membered cycloalkenyl, 3-14 membered heterocyclyl, 6-10 membered aryl, or 5-10 membered heteroaryl is optionally substituted with C₁₋₆ alkyl, —OH, halogen, —C(O)R^{2a}, or —C(O)NR^{2b}R^{2c}; wherein R^{2a} is C₁₋₆ alkyl or —(CH₂)ᵣOCH₃, wherein r is 1, 2, or 3; wherein R^{2b} is H or C₁₋₆ alkyl; and wherein R^{2e} is H or C1.6 alkyl; and

R³ and R⁴ are independently H or C₁₋₆ alkyl; wherein at least one of R³ and R⁴ is not H; or R³ and R⁴ together with the atom to which they are attached combine to form a 3-6 membered cycloalkyl; and

wherein the proviso of Formula (I) also applies to Formula (I-a).

Another aspect of the present disclosure relates to compounds of Formula (V):

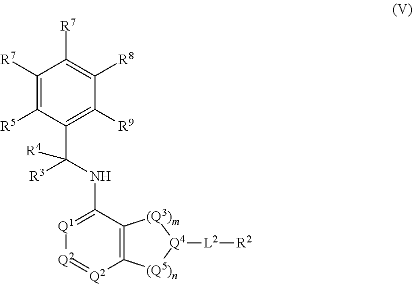

or a pharmaceutically acceptable salt, solvate, isomer, prodrug, or tautomer thereof, wherein:

L², Q¹, Q², Q³, Q⁴, Q⁵, m, n, R¹, R², R³ and R⁴ are as defined in Formula (I);

R⁵, R⁶, R⁷, R⁸, and R⁹ are independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OH, halogen, —NO₂, —CN, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, halogen, —NO₂, oxo, —CN, —R¹⁰, —OR¹⁰, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, 3-8 membered cycloalkyl, 3-14 membered heterocyclyl, 6-10 membered aryl, or 5-10 membered heteroaryl, or any two adjacent R⁵, R⁶, R⁷, R⁸, and R⁹ form a 3-14 membered fused ring;

R¹⁰, R¹¹, and R¹² are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR 3, —SR¹¹, halogen, —NR¹³R¹⁴, —NO₂, and —CN; and

R¹³ and R¹⁴ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN; and

wherein the proviso of Formula (I) also applies to Formula (V).

Another aspect of the present disclosure relates to compounds of Formula (V-a):

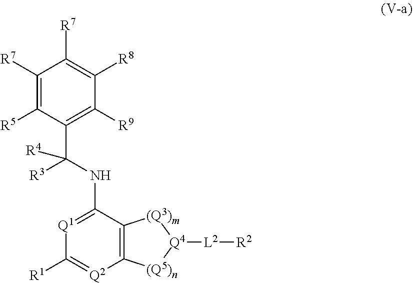

or a pharmaceutically acceptable salt, solvate, isomer, prodrug, or tautomer thereof, wherein:

Q¹, Q³, Q⁴, Q⁵, m, n, R², R³, R⁴, R⁵, R⁶, R⁷, R⁸, R⁹, R¹⁰, R¹¹, R¹², R¹³ and R¹⁴ are as defined in Formula (V);

Q² is CH or N;

wherein at least one of Q¹, Q², Q³, Q⁴, and Q⁵ is N, NR^{QN}, O, or SO₂;

R¹ is selected from the group consisting of H, halogen, C₁₋₆ alkyl, cyclopropyl, —CN, and —OR^{1a}; wherein R^{1a} is H or C₁₋₆ alkyl; and

L² is selected from the group consisting of a bond, —C(O)—, —C(O)O—, —C(O)NH(CH₂)ₒ—, —S(O)₂—, —C(O)(CH₂)ₚ—, —(CH₂)ₚ—, or —O—; wherein o is 0, 1, or 2; and wherein p is a number from 1 to 6; and wherein the proviso of Formula (I) also applies to Formula (V-a).

Another aspect of the present disclosure relates to compounds of Formula (VI):

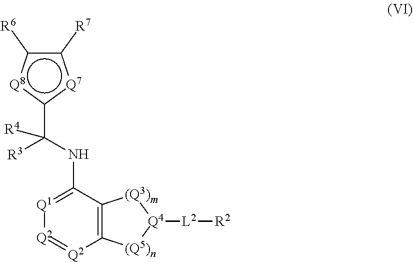

or a pharmaceutically acceptable salt, solvate, isomer, prodrug, or tautomer thereof, wherein:

L², Q¹, Q², Q³, Q⁴, Q⁵, m, n, R¹, R², R³, and R⁴ are as defined in Formula (I);

Q⁷ and Q⁸ are each independently CH, N, NH, O, or S, provided at least one of Q⁷ and Q⁸ is N, NH, O, or S;

R⁶ and R⁷ are independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OH, halogen, —NO₂, —CN, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, halogen, —NO₂, oxo, —CN, —R¹⁰, —OR¹⁰, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, 3-8 membered cycloalkyl, 3-14 membered heterocyclyl, 6-10 membered aryl, or 5-10 membered heteroaryl,

R¹⁰, R¹¹, and R¹² are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR¹³, —SR¹³, halogen, —NR¹³R¹⁴, —NO₂, and —CN; and

R¹³ and R¹⁴ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN; and

wherein the proviso of Formula (I) also applies to Formula (VI).

Another aspect of the present disclosure relates to compounds of Formula (VI-a).

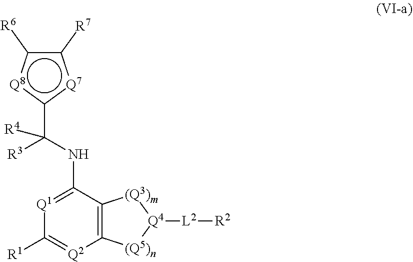

wherein L², Q¹, Q², Q³, Q⁴, Q⁵, Q⁷, Q⁸, R¹, R², R³, R⁴, R⁶, and R⁷ are as defined in Formula (VI), and wherein the proviso of Formula (I) also applies to Formula (VI-a).

Another aspect of the present disclosure relates to a pharmaceutical composition comprising a compound, or a pharmaceutically acceptable salt, solvate, hydrate, tautomer, or isomer thereof, as set forth above and a pharmaceutically acceptable carrier.

Another aspect of the present disclosure relates to a method of inhibiting SOS1 in a subject, comprising administering to the subject a compound, or a pharmaceutically acceptable salt, solvate, hydrate, tautomer, or isomer thereof, or a pharmaceutical composition, as set forth above.

Another aspect of the present disclosure relates to a method of inhibiting the interaction of SOS1 and a RAS-family protein in a cell or inhibiting the interaction of SOS1 and RAC1 in a cell, comprising administering to the cell a compound, or a pharmaceutically acceptable salt, solvate, hydrate, tautomer, or isomer thereof, or a pharmaceutical composition, as set forth above.

Another aspect of the present disclosure relates to a method of treating or preventing a disease, wherein treating or preventing the disease is characterized by inhibition of the interaction of SOS1 and a RAS-family protein or by inhibition of the interaction of SOS1 and RAC1, the method comprising administering to a subject in need thereof an effective amount of a compound, or a pharmaceutically acceptable salt, solvate, hydrate, tautomer, or isomer thereof, or a pharmaceutical composition, as set forth above.

Another aspect of the present disclosure relates to a method of treating or preventing cancer in a subject in need thereof, comprising administering to the subject an effective amount of a compound, or a pharmaceutically acceptable salt, solvate, hydrate, tautomer, or isomer thereof, or a pharmaceutical composition, as set forth above.

Another aspect of the present disclosure relates to a compound, or a pharmaceutically acceptable salt, solvate, hydrate, tautomer, or isomer thereof, or a pharmaceutical composition, as set forth above for use as a medicament.

Another aspect of the present disclosure relates to the use of the compound, or a pharmaceutically acceptable salt, solvate, hydrate, tautomer, or isomer thereof, or a pharmaceutical composition, as set forth above in the manufacture of a medicament for use in inhibiting the binding of hSOS1 to H- or N- or K-RAS including their clinically known mutations and which inhibits the nucleotide exchange reaction catalyzed by hSOS1 in the presence of a concentration of 20 μM or lower, but which are substantially inactive against EGFR-kinase at concentrations of 20 μM or lower.

Another aspect of the present disclosure relates to the use the compound, or a pharmaceutically acceptable salt, solvate, hydrate, tautomer, or isomer thereof, or a pharmaceutical composition, as set forth above in the manufacture of a medicament for use inhibiting the binding of hSOS1 specifically to K-RAS G12C protein and which inhibits the nucleotide exchange reaction catalyzed by hSOS1 in the presence of a concentration of 20 μM or lower, but which are substantially inactive against EGFR-kinase at concentrations of 20 μM or lower.

The present disclosure also provides a compound, or a pharmaceutically acceptable salt, prodrug, solvate, hydrate, tautomer, or isomer thereof, or a pharmaceutical composition, as set forth above that is useful in inhibiting SOS1.

### BRIEF DESCRIPTION OF THE DRAWINGS

FIG. 1A is a graph displaying the efficacy of repeated daily dosing of Compound A at 50 and 250 mg/kg po and MRTX1257 at 10 mg/kg on tumor cell growth in vivo in a NSCLC NCI-H358 xenograft model using female balb/c athymic nude mice.

FIG. 1B is a graph displaying mice body weight change associated with efficacy study of FIG. 1A.

FIG. 1C depicts the Structure of MRTX1257.

### DETAILED DESCRIPTION OF THE DISCLOSURE

The details of the present disclosure are set forth in the accompanying description below. Although methods and materials similar or equivalent to those described herein can be used in the practice or testing of the present disclosure, illustrative methods and materials are now described. Other features, objects, and advantages of the present disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which the present disclosure belongs. All patents and publications cited in this specification are incorporated herein by reference in their entireties.

### Terms

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise. The use of the term “or” is used to mean “and/or” unless explicitly indicated to refer to alternatives only or the alternative are mutually exclusive, although the disclosure supports a definition that refers to only alternatives and “and/or.”

As used herein, the term “about” is used to indicate that a value includes the standard deviation of error for the device or method being employed to determine the value. In certain embodiments, the term “about” refers to a range of values that fall within 25%, 20%, 19%, 18%, 17%1, 16%%, 15%, 14%, 13%, %12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

By “optional” or “optionally,” it is meant that the subsequently described event or circumstance may or may not occur, and that the description includes instances where the event or circumstance occurs and instances in which it does not. For example, “optionally substituted aryl” encompasses both “aryl” and “substituted aryl” as defined herein. It will be understood by those ordinarily skilled in the art, with respect to any group containing one or more substituents, that such groups are not intended to introduce any substitution or substitution patterns that are sterically impractical, synthetically non-feasible, and/or inherently unstable.

The term “optionally substituted” unless otherwise specified means that a group may be unsubstituted or substituted by one or more (e.g., 0, 1, 2, 3, 4, or 5 or more, or any range derivable therein) of the substituents listed for that group in which said substituents may be the same or different. In an embodiment, an optionally substituted group has 1 substituent. In another embodiment, an optionally substituted group has 2 substituents. In another embodiment, an optionally substituted group has 3 substituents. In another embodiment, an optionally substituted group has 4 substituents. In another embodiment, an optionally substituted group has 5 substituents. For instance, an alkyl group that is optionally substituted can be a fully saturated alkyl chain (i.e., a pure hydrocarbon). Alternatively, the same optionally substituted alkyl group can have substituents different from hydrogen. For instance, it can, at any point along the chain be bonded to a halogen atom, a hydroxyl group, or any other substituent described herein. Thus the term “optionally substituted” means that a given chemical moiety has the potential to contain other functional groups, but does not necessarily have any further functional groups.

As used herein, “alkyl” may mean a straight chain or branched saturated chain having from 1 to 10 carbon atoms. Representative saturated alkyl groups include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, n-pentyl, isopentyl, neopentyl, n-hexyl and the like, and longer alkyl groups, such as heptyl, and octyl and the like. An alkyl group can be unsubstituted or substituted. Alkyl groups containing three or more carbon atoms may be straight or branched. As used herein, “lower alkyl” means an alkyl having from 1 to 6 carbon atoms.

As used herein, the term “heteroalkyl” refers to an “alkyl” group (as defined herein), in which at least one carbon atom has been replaced with a heteroatom (e.g., an O, N, or S atom). The heteroatom may appear in the middle or at the end of the radical.

The term “alkenyl” means an aliphatic hydrocarbon group containing a carbon-carbon double bond and which may be straight or branched having about 2 to about 6 carbon atoms in the chain. Certain alkenyl groups have 2 to about 4 carbon atoms in the chain. Branched means that one or more lower alkyl groups such as methyl, ethyl, or propyl are attached to a linear alkenyl chain. Exemplary alkenyl groups include ethenyl, propenyl, n-butenyl, and i-butenyl. A C₂-C₆ alkenyl group is an alkenyl group containing between 2 and 6 carbon atoms.

The term “alkynyl” means an aliphatic hydrocarbon group containing a carbon-carbon triple bond and which may be straight or branched having about 2 to about 6 carbon atoms in the chain. Certain alkynyl groups have 2 to about 4 carbon atoms in the chain. Branched means that one or more lower alkyl groups such as methyl, ethyl, or propyl are attached to a linear alkynyl chain. Exemplary alkynyl groups include ethynyl, propynyl, n-butynyl, 2-butynyl, 3-methylbutynyl, and n-pentynyl. A C₂-C₆ alkynyl group is an alkynyl group containing between 2 and 6 carbon atoms.

As used herein, the term “halo” or “halogen” means a fluoro, chloro, bromo, or iodo group.

The term “oxo” as used herein refers to an “═O” group. When an oxo group is bonded to a carbon atom, it can also be abbreviated herein as C(O) or as C═O. An oxo group can also be bonded to a sulfur atom (e.g., S═O and S(O)₂) or at phosphorous atom (e.g., P═O, PO₂, PO₃, PO₄, etc.).

The term “imine” as used herein refers to an “═N” group. When an imine is bonded to a carbon atom, it can also be abbreviated herein as C═N. Nitrogen can also be double bonded to sulfur, e.g., S═N, which is referred to as a thioimine.

The term “annular atoms” used in conjunction with terms relating to ring systems described herein (e.g., cycloalkyl, cycloalkenyl, aryl, heterocyclyl, and heteroaryl) refers to the total number of ring atoms present in the system. “Annular atoms” therefore does not include the atoms present in a substituent attached to the ring. Thus, the number of “annular atoms” includes all atoms present in a fused ring. For example, a 2-indolyl ring,

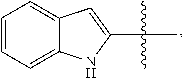 [SMILES: *c1cc2ccccc2[nH]1]

is considered a 5-membered heteroaryl, but is also a heteroaryl containing 9 annular atoms. In another example, pyridine is considered a 6-membered heteroaryl, and is a heteroaryl containing 6 annular atoms.

“Cycloalkyl” refers to a single saturated all carbon ring having 3 to 20 annular carbon atoms (i.e., C₃-C₂₀ cycloalkyl), for example from 3 to 15 annular atoms, for example, from 3 to 12 annular atoms. In certain embodiments, the cycloalkyl group is either monocyclic (“monocyclic cycloalkyl”) or contains a fused, bridged or spiro ring system such as a bicyclic system (“bicyclic cycloalkyl”) and can be saturated. “Cycloalkyl” includes ring systems where the cycloalkyl ring, as defined above, is fused with one or more cycloalkyl, cycloalkenyl, heterocyclyl, aryl or heteroaryl groups, wherein the point of attachment is on a cycloalkyl ring, and, in such instances, the number of carbon atoms recited continues to designate the number of carbons in the cycloalkyl ring containing the point of attachment. Examples of cycloalkyl groups include cyclohexyl, cycloheptyl, 2-adamantyl

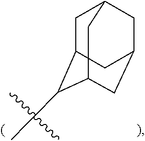 [SMILES: *C1C2CC3CC(C2)CC1C3]

2-(2,3-dihydro-1H-indene)

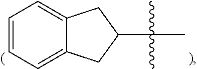 [SMILES: *C1Cc2ccccc2C1]

and 9-fluorenyl

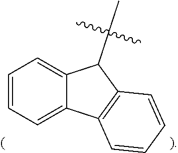 [SMILES: *C1c2ccccc2-c2ccccc21]

As noted above, cycloalkyl rings can be further characterized by the number of annular atoms. For example, a cyclohexyl ring is a C₆ cycloalkyl ring with 6 annular atoms, while 2-(2,3-dihydro-1H-indene) is a C₅ cycloalkyl ring with 9 annular atoms. Also, for example, 9-fluorenyl is a C₅ cycloalkyl ring with 13 annular atoms and 2-adamantyl is a C₆ cycloalkyl with 10 annular atoms.

As used herein, the term “cycloalkenyl” may refer to a partially saturated, monocyclic, fused or spiro polycyclic, all carbon ring having from 3 to 18 carbon atoms per ring and contains at least one double bond. “Cycloalkenyl” includes ring systems where the cycloalkenyl ring, as defined above, is fused with one or more cycloalkyl, cycloalkenyl, heterocyclyl, aryl or heteroaryl groups, wherein the point of attachment is on a cycloalkenyl ring, and, in such instances, the number of carbon atoms recited continues to designate the number of carbons in the cycloalkenyl ring containing the point of attachment. Cycloalkenyl rings can be further characterized by the number of annular atoms. Examples of cycloalkenyl include 1-cyclohex-1-enyl and cyclopent-1-enyl.

The term “aryl” as used herein refers to a single all carbon aromatic ring or a multiple condensed all carbon ring system wherein at least one of the rings is aromatic. For example, in certain embodiments, an aryl group has 5 to 20 annular carbon atoms, 5 to 14 annular carbon atoms, or 5 to 12 annular carbon atoms. Aryl also includes multiple condensed ring systems (e.g., ring systems comprising 2, 3 or 4 rings) having about 9 to 20 carbon atoms in which at least one ring is aromatic and wherein the other rings may be aromatic or not aromatic (i.e., cycloalkyl). “Aryl” includes ring systems where the aryl ring, as defined above, is fused with one or more cycloalkyl, cycloalkenyl, heterocyclyl, aryl or heteroaryl groups, and wherein the point of attachment is on an aryl ring, and, in such instances, the number of carbon atoms recited continues to designate the number of carbon atoms in the aryl ring containing the point of attachment. Examples of aryl groups include phenyl and 5-(2,3-dihydro-TH-indene):

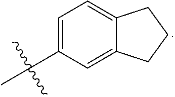 [SMILES: *c1ccc2c(c1)CCC2]

As noted above, aryl rings can be further characterized by the number of annular atoms. For example, phenyl is a C₆ aryl with 6 annular atoms, while 5-(2,3-dihydro-TH-indene) is a C₆ aryl with 9 annular atoms.

“Heterocyclyl” as used herein refers to a single saturated or partially unsaturated non-aromatic ring or a non-aromatic multiple ring system (including fused and spiro polycyclic) that has at least one heteroatom in the ring (at least one annular heteroatom selected from oxygen, nitrogen, phosphorus, and sulfur). Unless otherwise specified, a heterocyclyl group has from 5 to about 20 annular atoms, for example from 5 to 15 annular atoms, for example from 5 to 10 annular atoms. Thus, the term includes single saturated or partially unsaturated rings (e.g., 3, 4, 5, 6 or 7-membered rings) having from about 1 to 6 annular carbon atoms and from about 1 to 3 annular heteroatoms selected from the group consisting of oxygen, nitrogen, phosphorus, and sulfur in the ring. The term also includes single saturated or partially unsaturated rings (e.g., 5, 6, 7, 8, 9, or 10-membered rings) having from about 4 to 9 annular carbon atoms and from about 1 to 3 annular heteroatoms selected from the group consisting of oxygen, nitrogen, phosphorus, and sulfur in the ring. “Heterocyclyl” includes ring systems where the heterocyclyl ring, as defined above, is fused with one or more cycloalkyl, cycloalkenyl, heterocyclyl, aryl or heteroaryl groups, wherein the point of attachment is on a heterocyclic ring, and, in such instances, the number of ring members recited continues to designate the number of annular atoms in the heterocyclic ring containing the point of attachment. Heterocyclic rings can be further characterized by the number of annular atoms. Examples of heterocyclic groups include piperidinyl (6-membered heterocycle with 6 annular atoms), azepanyl (7-membered heterocycle with 7 annular atoms), and 3-chromanyl (6-membered heterocycle with 10 annular atoms)

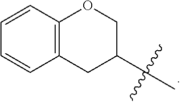 [SMILES: *C1COc2ccccc2C1]

The term “heteroaryl” as used herein refers to a single aromatic ring that has at least one atom other than carbon in the ring, wherein the atom is selected from the group consisting of oxygen, nitrogen and sulfur; the term also includes multiple condensed ring systems that have at least one such aromatic ring. Thus, the term includes single heteroaryl rings of from about 1 to 10 annular carbon atoms and about 1-5 annular heteroatoms selected from the group consisting of oxygen, nitrogen and sulfur in the rings. The sulfur and nitrogen atoms may also be present in an oxidized form provided the ring is aromatic. “Heteroaryl” includes ring systems where the heteroaryl ring, as defined above, is fused with one or more cycloalkyl, cycloalkenyl, heterocyclyl, aryl or heteroaryl groups, wherein the point of attachment is on a heteroaryl ring, and, in such instances, the number of ring members continues to designate the number of ring members in the heteroaryl ring containing the point of attachment. Heteroaryl rings can be further characterized by the number of annular atoms. For example, pyridine is a 6-membered heteroaryl having 6 annular atoms.

The disclosure also includes pharmaceutical compositions comprising an effective amount of a disclosed compound and a pharmaceutically acceptable carrier. Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, sethionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

The term “tautomers” refers to a set of compounds that have the same number and type of atoms, but differ in bond connectivity and are in equilibrium with one another. A “tautomer” is a single member of this set of compounds. Typically a single tautomer is drawn but it is understood that this single structure is meant to represent all possible tautomers that might exist. Examples include enol-ketone tautomerism. When a ketone is drawn it is understood that both the enol and ketone forms are part of the present disclosure.

Compounds of the present disclosure can also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the present disclosure can be replaced or substituted with isotopes of the atoms in natural or non-natural abundance. In some embodiments, the compound comprises at least one deuterium atom. For example, one or more hydrogen atoms in a compound of the present disclosure can be replaced or substituted by deuterium. In some embodiments, the compound comprises two or more deuterium atoms. In some embodiments, the compound comprises 1, 2, 3, 4, 5, 6, 7, 8, 9, 10, 11 or 12 deuterium atoms. Synthetic methods for including isotopes into organic compounds are known in the art.

The term “prodrug,” as used in this disclosure, means a compound which is convertible in vivo by metabolic means (e.g., by hydrolysis) to a disclosed compound. Furthermore, as used herein a prodrug is a drug which is inactive in the body, but is transformed in the body typically either during absorption or after absorption from the gastrointestinal tract into the active compound. The conversion of the prodrug into the active compound in the body may be done chemically or biologically (i.e., using an enzyme).

The term “solvate” refers to a complex of variable stoichiometry formed by a solute and solvent. Such solvents for the purpose of the present disclosure may not interfere with the biological activity of the solute. Examples of suitable solvents include, but are not limited to, water, MeOH, EtOH, and AcOH. Solvates wherein water is the solvent molecule are typically referred to as hydrates. Hydrates include compositions containing stoichiometric amounts of water, as well as compositions containing variable amounts of water.

The term “isomer” refers to compounds that have the same composition and molecular weight but differ in physical and/or chemical properties. The structural difference may be in constitution (geometric isomers) or in the ability to rotate the plane of polarized light (stereoisomers). With regard to stereoisomers, the compounds herein may have one or more asymmetric carbon atom and may occur as racemates, racemic mixtures and as individual enantiomers or diastereomers.

The term “stereoisomers” refers to the set of compounds which have the same number and type of atoms and share the same bond connectivity between those atoms, but differ in three dimensional structure. The term “stereoisomer” refers to any member of this set of compounds. For instance, a stereoisomer may be an enantiomer or a diastereomer.

The term “enantiomers” refers to a pair of stereoisomers which are non-superimposable mirror images of one another. The term “enantiomer” refers to a single member of this pair of stereoisomers. The term “racemic” refers to a 1:1 mixture of a pair of enantiomers.

The term “diastereomers” refers to the set of stereoisomers which cannot be made superimposable by rotation around single bonds. For example, cis- and trans-double bonds, endo- and exo-substitution on bicyclic ring systems, and compounds containing multiple stereogenic centers with different relative configurations are considered to be diastereomers. The term “diastereomer” refers to any member of this set of compounds. In some examples presented, the synthetic route may produce a single diastereomer or a mixture of diastereomers.

An “effective amount” when used in connection with a compound is an amount effective for treating or preventing a disease in a subject as described herein.

The term “carrier”, as used in this disclosure, encompasses excipients and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treating” with regard to a subject, refers to improving at least one symptom of the subject's disorder. Treating includes curing, improving, or at least partially ameliorating the disorder.

The term “prevent” or “preventing” with regard to a subject refers to keeping a disease or disorder from afflicting the subject. Preventing includes prophylactic treatment. For instance, preventing can include administering to the subject a compound disclosed herein before a subject is afflicted with a disease and the administration will keep the subject from being afflicted with the disease.

The terms “inhibiting” and “reducing,” or any variation of these terms, includes any measurable or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of activity (e.g., SOS1:Ras-family protein binding activity) compared to normal.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

A “patient” or “subject” is a mammal, e.g., a human, mouse, rat, guinea pig, dog, cat, horse, cow, pig, or non-human primate, such as a monkey, chimpanzee, baboon or rhesus.

### Compounds of Disclosed Formulae

In some embodiments, the present disclosure relates to compounds of the following formula:

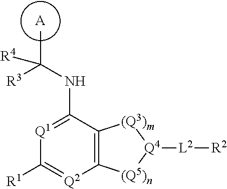

or a pharmaceutically acceptable salt, solvate, isomer, prodrug, or tautomer thereof, wherein:

Q¹ and Q² are independently CH or N;
each Q³ and Q⁵ are independently C(R^{QC})₂, NR^{QN}, CO, O, or SO₂, wherein each R^{QC} is independently H, F, Cl, Br, or aryl, and wherein each R^{QN} is independently H, C₁₋₆ alkyl, or aryl;

Q⁴ is CH or N;

wherein at least one of Q¹, Q², Q³, Q⁴, and Q⁵ is N, NR^{QN}, O, or SO₂;

m is 0, 1, 2, or 3;

n is 0, 1, 2, or 3;

wherein when m is 0, then n is not 0;

R¹ is H, halogen, C₁₋₆ alkyl, 3-membered cycloalkyl, —CN, or —OR^{1a}; wherein R^{1a} is H or C₁₋₆ alkyl;

L² is a bond, —C(O)—, —C(O)O—, —C(O)NH(CH₂)ₒ—, —S(O)₂—, —C(O)(CH₂)ₚ—, —(CH₂)ₚ—, or —O—; wherein o is 0, 1, or 2; and wherein p is a number from 1 to 6;

R² is H, —(CH₂)_{q}CH₃, cycloalkyl, cycloalkenyl, heterocyclyl, aryl, heteroaryl; wherein q is a number from 1 to 5; wherein each cycloalkyl, cycloalkenyl, heterocyclyl, aryl, or heteroaryl is optionally substituted with one or more C₁₋₆ alkyl, —OH, halogen, —C(O)R^{2a}, or —C(O)NR^{2b}R^{2c}; wherein R^{2a} is C₁₋₆ alkyl or —(CH₂)ᵣOCH₃, wherein r is 1, 2, or 3; wherein R^{2b} is H or C₁₋₆ alkyl; and wherein R^{2c} is H or C₁₋₆ alkyl;

R³ and R⁴ are independently selected from the group consisting of H and C₁₋₆ alkyl; wherein at least one of R³ and R⁴ is not H; or R³ and R⁴ together with the atom to which they are attached combine to form a 3-6 membered cycloalkyl; and

A is an optionally substituted 6-membered aryl or an optionally substituted 5-6 membered heteroaryl;

with the proviso that when

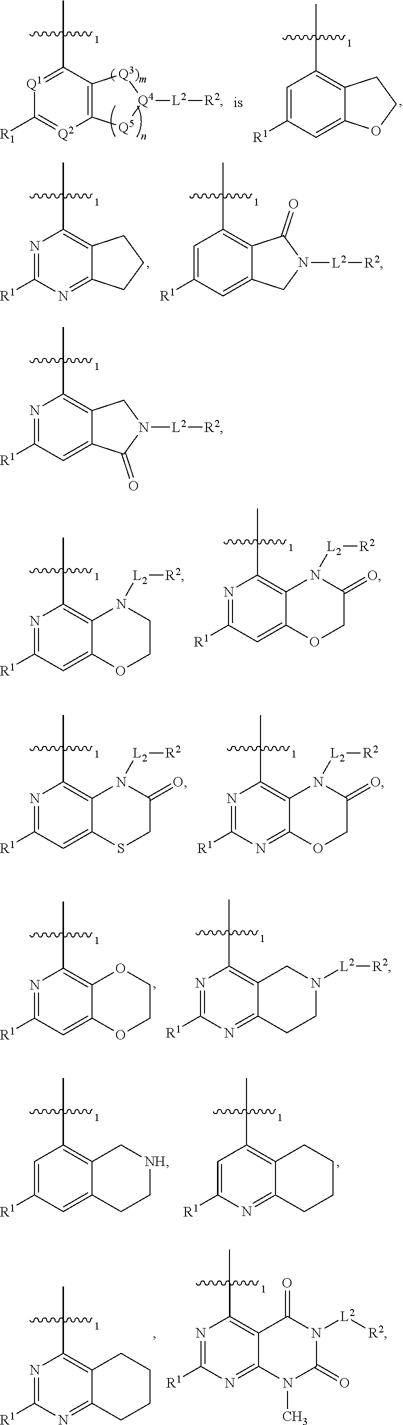

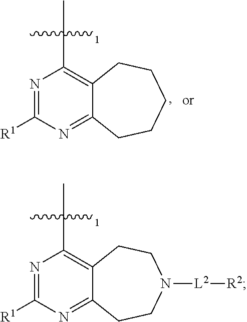

then R¹ is not H.

In other embodiments, the present disclosure relates to compounds of the following formula:

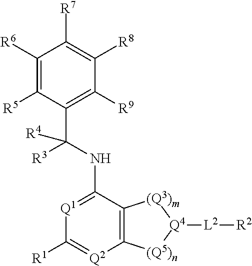

or a pharmaceutically acceptable salt, solvate, isomer, prodrug, or tautomer thereof, wherein:

Q¹ and Q² are independently CH or N;

each Q³ and Q⁵ are independently C(R^{QC})₂, NR^{QN}, CO, O, or SO₂, wherein each R^{QC} is independently H, F, Cl, Br, or aryl, and wherein each R^{QN} is independently H, C₁₋₆ alkyl, or aryl;

Q⁴ is CH or N;

wherein at least one of Q¹, Q², Q³, Q⁴, and Q⁵ is N, NR^{QN}, O, or SO₂;

m is 0, 1, 2, or 3;

n is 0, 1, 2, or 3;

wherein when m is 0, then n is not 0;

R¹ is H, halogen, C₁₋₆ alkyl, 3-membered cycloalkyl, —CN, or —OR^{1a}; wherein R^{1a} is H or C₁₋₆ alkyl;

L² is a bond, —C(O)—, —C(O)O—, —C(O)NH(CH₂)ₒ—, —S(O)₂—, —C(O)(CH₂)ₚ—, —(CH₂)ₚ—, or —O—; wherein o is 0, 1, or 2; and wherein p is a number from 1 to 6;

R² is H, —(CH₂)_{q}CH₃, cycloalkyl, cycloalkenyl, heterocyclyl, aryl, heteroaryl; wherein q is a number from 1 to 5; wherein each cycloalkyl, cycloalkenyl, heterocyclyl, aryl, or heteroaryl is optionally substituted with one or more C₁₋₆ alkyl, —OH, halogen, —C(O)R^{2a}, or —C(O)NR^{2b}R^{2c}; wherein R^{2a} is C₁₋₆ alkyl or —(CH₂)ᵣOCH₃, wherein r is 1, 2, or 3; wherein R^{2b} is H or C₁₋₆ alkyl; and wherein R^{2c} is H or C₁₋₆ alkyl;

R³ and R⁴ are independently selected from the group consisting of H and C₁₋₆ alkyl; wherein at least one of R³ and R⁴ is not H; or R³ and R⁴ together with the atom to which they are attached combine to form a 3-6 membered cycloalkyl; and

R⁵, R⁶, R⁷, R⁸, and R⁹ are independently selected from the group consisting of H, D, C₁₋₆ alkyl, C₂₋₆ alkenyl, C₄₋₈ cycloalkenyl, C₂₋₆ alkynyl, C₃₋₈ cycloalkyl, —OH, halogen, —NO₂, —CN, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, —C(O)R¹⁰, or —CO₂R¹⁰, wherein each alkyl, alkenyl, cycloalkenyl, alkynyl, or cycloalkyl is optionally substituted with one or more —OH, halogen, —NO₂, oxo, —CN, —R¹⁰, —OR¹⁰, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹R², —NR¹⁰S(O)R¹¹, heterocycle, aryl, or heteroaryl;

R¹⁰, R¹¹, and R¹² are independently, at each occurrence, H, D, C₁₋₆ alkyl, C₂₋₆ alkenyl, C₄₋₈ cycloalkenyl, C₂₋₆ alkynyl, C₃₋₈ cycloalkyl, a monocyclic 3-12 membered heterocycle, a polycyclic 3-12 membered heterocycle, —OR¹³, —SR¹³, halogen, —NR¹³R¹⁴, —NO₂, or —CN;

R¹³ and R¹⁴ are independently, at each occurrence, H, D, C₁₋₆ alkyl, C₂₋₆ alkenyl, C₄₋₈ cycloalkenyl, C₂₋₆ alkynyl, C₃₋₈ cycloalkyl, a monocyclic 3-12 membered heterocycle, or a polycyclic 3-12 membered heterocycle, wherein each alkyl, alkenyl, cycloalkenyl, alkynyl, cycloalkyl, or heterocycle is optionally substituted with one or more —OH, —SH, —NH₂, —NO₂, or —CN; with the proviso that when

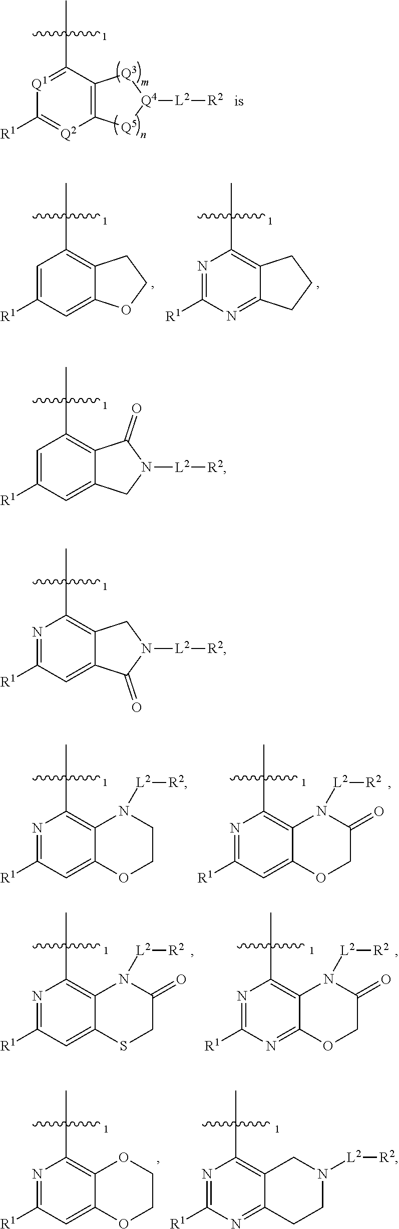

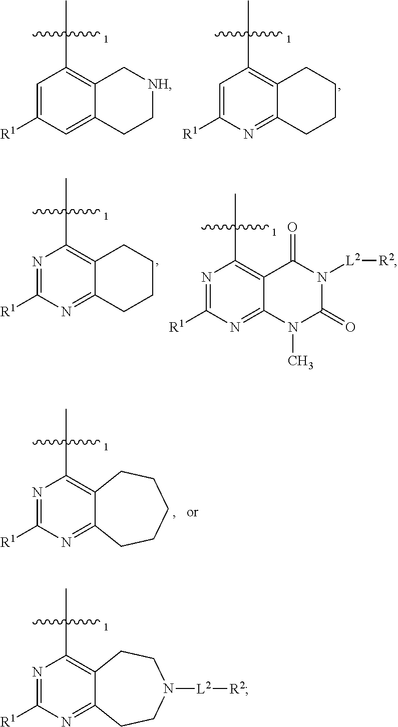

then R¹ is not H.

### Additional Compounds of Disclosed Formulae

The present disclosure provides for compounds of Formula (I),

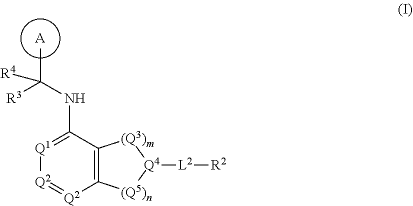

or a pharmaceutically acceptable salt, solvate, isomer, prodrug, or tautomer thereof, wherein:

Q¹ is CH or N;

Q⁴ is CH, C, or N;

each Q² is independently C—R¹ or N, wherein one Q² is N and the other Q² is C—R¹;

each Q³ and Q⁵ are independently C(R^{QC})₂, NR^{QN}, CO, O, S, or SO₂, wherein each R^{QC} is independently H, F, Cl, Br, or 6-10 membered aryl, and wherein each R^{QN} is independently H, C₁₋₆ alkyl, or 6-10 membered aryl;

wherein at least one of Q¹, Q², Q³, Q⁴, and Q⁵ is N, NR^{QN}, O, or SO₂;

m is 0, 1, 2, or 3;

n is 0, 1, 2, or 3;

wherein when m is 0, then n is not 0;

R¹ is selected from the group consisting of H, C₁₋₆ alkyl, halogen, —CONHR^{1a}, —NHR^{1a}, —OR^{1a}, cyclopropyl, azetidinyl, and —CN; wherein each C₁₋₆ alkyl and azetidinyl is optionally substituted with halogen, R^{1a}, —NHR^{1a}, or —OR^{1a}; wherein R^{1a} is H, C₁₋₆ alkyl, cyclopropyl, 3-6 membered heterocyclyl, or C₁₋₆ haloalkyl;

L² is selected from the group consisting of a bond, —C(O)—, —C(O)O—, —C(O)NH(CH₂)ₒ—, —S(O)₂—,

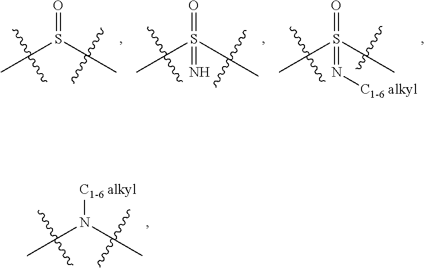

—C(O)(CH₂)ₚ—, —(CH₂)ₚ—, and —O—; wherein o is 0, 1, or 2; and wherein p is a number from 1 to 6;

R² is selected from the group consisting of H, C₁₋₆ alkyl, —NR^{2b}R^{2c}, —OR^{2a}, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl are independently optionally substituted with C₁₋₆ alkyl, C₁₋₆ haloalkyl, C₁₋₆ hydroxyalkyl, C₁₋₆ methoxyalkyl, —OH, —OR^{2a}, oxo, ═N, halogen, —C(O)R^{2a}, —C(O)OR^{2a}, —C(O)NR^{2b}R^{2c}, —SO₂R^{2a}, —CN, —NR^{2b}R^{2c}, 3-6 membered cycloalkyl, 3-7 membered heterocyclyl, 6-10 membered aryl, or 5-10 membered heteroaryl;
wherein R^{2a} is H, C₁₋₆ alkyl, C₁₋₆ haloalkyl, 3-7 membered heterocyclyl, or —(CH₂)ᵣOCH₃, wherein r is 1, 2, or 3;
wherein R^{2b} is H or C₁₋₆ alkyl;
wherein R^{2c} is H or C₁₋₆ alkyl;

R³ and R⁴ are independently H or C₁₋₆ alkyl optionally substituted with halo or —OH; wherein at least one of R³ and R⁴ is H or wherein R³ and R⁴ together with the atom to which they are attached combine to form a 3-6 membered cycloalkyl; and

A is an optionally substituted 6-membered aryl or an optionally substituted 5-6 membered heteroaryl;

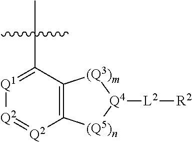

with the proviso that when

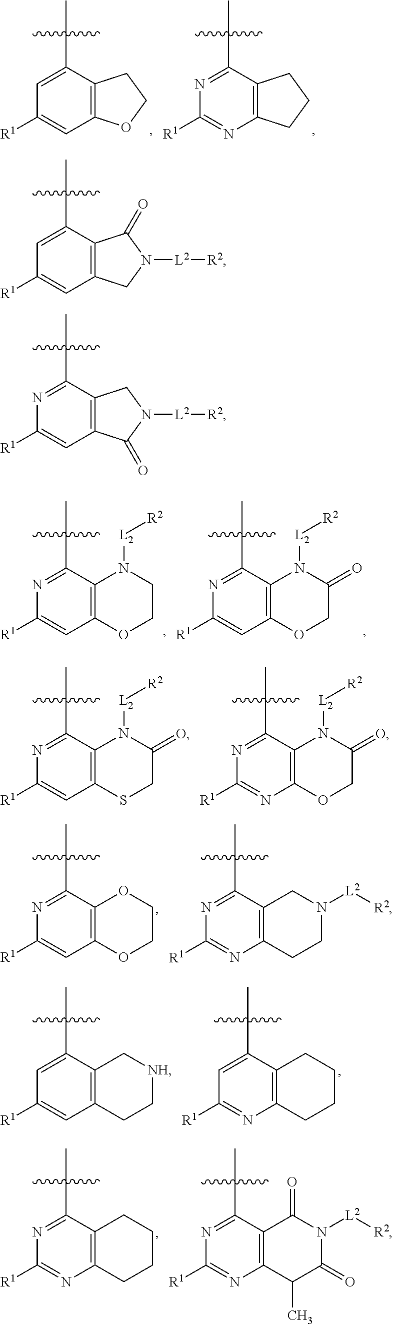

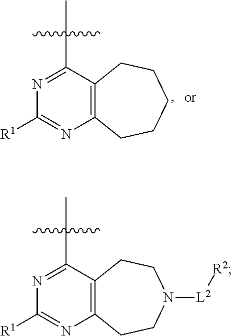

then R¹ is not H.

The present disclosure provides for compounds of Formula (I-a),

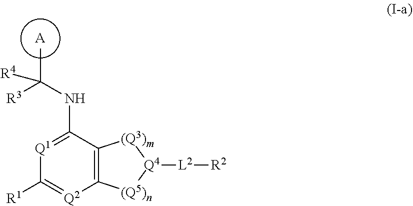

or a pharmaceutically acceptable salt, solvate, isomer, prodrug, or tautomer thereof, wherein:

Q¹, Q³, Q⁴, Q⁵, m, n and A are as defined in Formula (I);

Q² is CH or N;

wherein at least one of Q¹, Q², Q³, Q⁴, and Q⁵ is N, NR^{QN}, O, or SO₂;

R¹ is selected from the group consisting of H, halogen, C₁₋₆ alkyl, cyclopropyl, —CN, and —OR^{1a}; wherein R^{1a} is H or C₁₋₆ alkyl;

L² is selected from the group consisting of a bond, —C(O)—, —C(O)O—, —C(O)NH(CH₂)ₒ—, —S(O)₂—, —C(O)(CH₂)ₚ—, —(CH₂)ₚ—, or —O—; wherein o is 0, 1, or 2; and wherein p is a number from 1 to 6;

R² is selected from the group consisting of H, —(CH₂)_{q}CH₃, 3-14 membered cycloalkyl, 3-14 membered cycloalkenyl, 3-14 membered heterocyclyl, 6-10 membered aryl, and 5-10 membered heteroaryl; wherein q is a number from 1 to 5; wherein each 3-14 membered cycloalkyl, 3-14 membered cycloalkenyl, 3-14 membered heterocyclyl, 6-10 membered aryl, or 5-10 membered heteroaryl is optionally substituted with C₁₋₆ alkyl, —OH, halogen, —C(O)R^{2a}, or —C(O)NR^{2b}R^{2c}; wherein R^{2a} is C₁₋₆ alkyl or —(CH₂)ᵣOCH₃, wherein r is 1, 2, or 3; wherein R^{2b} is H or C₁₋₆ alkyl; and wherein R^{2c} is H or C1.6 alkyl; and

R³ and R⁴ are independently H or C₁₋₆ alkyl; wherein at least one of R³ and R⁴ is not H; or R³ and R⁴ together with the atom to which they are attached combine to form a 3-6 membered cycloalkyl.

The present disclosure provides for compounds of Formula (II-a), (II-b), or (II-c),

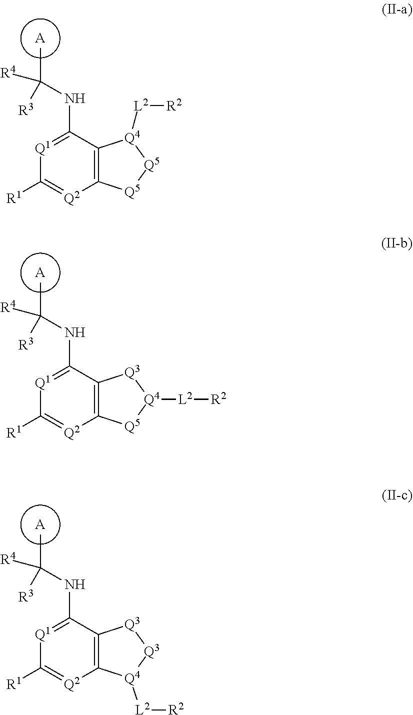

and pharmaceutically acceptable salts, prodrugs, solvates, hydrates, tautomers, or isomers thereof, wherein:

Q¹ and Q² are independently CH or N;

each Q³ and Q⁵ are independently C(R^{QC})₂, NR^{QN}, CO, O, or SO₂, wherein each R^{QC} is independently H, F, Cl, Br, or aryl, and wherein each R^{QN} is independently H, C₁₋₆ alkyl, or aryl (e.g., 6-10 membered aryl);

Q⁴ is CH or N;

wherein at least one of Q¹, Q², Q³, Q⁴, and Q⁵ is N, NR^{QN}, O, or SO₂;

R¹ is H, halogen, C₁₋₆ alkyl, cyclopropyl, —CN, or —OR^{1a}; wherein R^{1a} is H or C₁₋₆ alkyl;

L² is a bond, —C(O)—, —C(O)O—, —C(O)NH(CH₂)ₒ—, —S(O)₂—, —C(O)(CH₂)ₚ—, —(CH₂)ₚ—, or —O—; wherein o is 0, 1, or 2; and wherein p is a number from 1 to 6;

R² is H, —(CH₂)_{q}CH₃, cycloalkyl (e.g., 3-14 membered cycloalkyl), cycloalkenyl (e.g., 3-14 membered cycloalkenyl), heterocyclyl (e.g., 3-14 membered heterocyclyl), aryl (e.g., 6-10 membered aryl), heteroaryl (e.g., 5-10 membered heteroaryl); wherein q is a number from 1 to 5; wherein each cycloalkyl, cycloalkenyl, heterocyclyl, aryl, or heteroaryl is optionally substituted with one or more C₁₋₆ alkyl, —OH, halogen, —C(O)R^{2a}, or —C(O)NR^{2b}R^{2c}; wherein R^{2a} is C₁₋₆ alkyl or —(CH₂)ᵣOCH₃, wherein r is 1, 2, or 3; wherein R^{2b} is H or C₁₋₆ alkyl; and wherein R^{2c} is H or C₁₋₆ alkyl;

R³ and R⁴ are independently selected from the group consisting of H and C₁₋₆ alkyl; wherein at least one of R³ and R⁴ is not H; or R³ and R⁴ together with the atom to which they are attached combine to form a 3-6 membered cycloalkyl; and

A is an optionally substituted 6-membered aryl or an optionally substituted 5-6 membered heteroaryl;

with the proviso that when

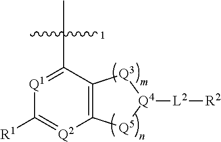

is

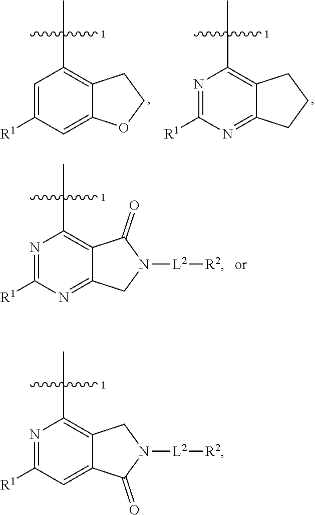

then R is not H.

The present disclosure provides for compounds of Formula (III-a), (III-b), (III-c), or (III-d)

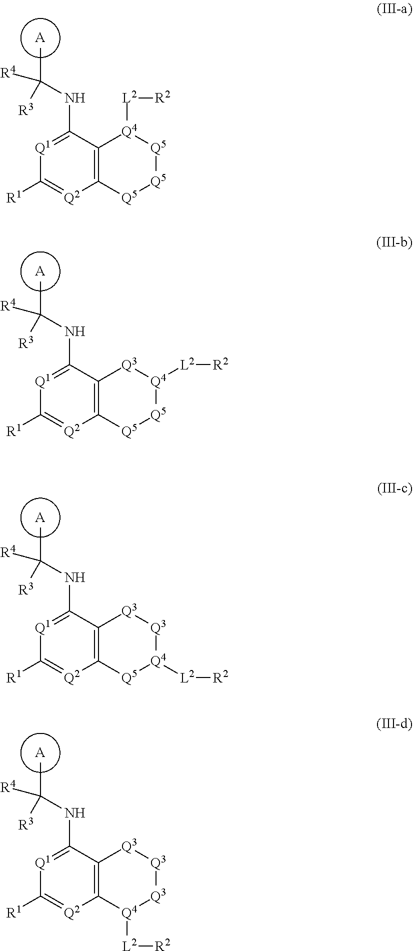

and pharmaceutically acceptable salts, prodrugs, solvates, hydrates, tautomers, or isomers thereof, wherein:

Q¹ and Q² are independently CH or N;

each Q³ and Q⁵ are independently C(R^{QC})₂, NR^{QN}, CO, O, or SO₂, wherein each R^{QC} is independently H, F, Cl, Br, or aryl, and wherein each R^{QN} is independently H, C₁₋₆ alkyl, or aryl (e.g., 6-10 membered aryl);

Q⁴ is CH or N;

wherein at least one of Q¹, Q², Q³, Q⁴, and Q⁵ is N, NR^{QN}, O, or SO₂;

R¹ is H, halogen, C₁₋₆ alkyl, cyclopropyl, —CN, or —OR^{1a}; wherein R^{1a} is H or C₁₋₆ alkyl;

L² is a bond, —C(O)—, —C(O)O—, —C(O)NH(CH₂)ₒ—, —S(O)₂—, —C(O)(CH₂)ₚ—, —(CH₂)ₚ—, or —O—; wherein o is 0, 1, or 2; and wherein p is a number from 1 to 6;

R² is H, —(CH₂)_{q}CH₃, cycloalkyl (e.g., 3-14 membered cycloalkyl), cycloalkenyl (e.g., 3-14 membered cycloalkenyl), heterocyclyl (e.g., 3-14 membered heterocyclyl), aryl (e.g., 6-10 membered aryl), heteroaryl (e.g., 5-10 membered heteroaryl); wherein q is a number from 1 to 5; wherein each cycloalkyl, cycloalkenyl, heterocyclyl, aryl, or heteroaryl is optionally substituted with one or more C₁₋₆ alkyl, —OH, halogen, —C(O)R^{2a}, or —C(O)NR^{2b}R^{2c}; wherein R^{2a} is C₁₋₆ alkyl or —(CH₂)ᵣOCH₃, wherein r is 1, 2, or 3; wherein R^{2b} is H or C₆ alkyl: and wherein R^{2c} is H or C₁₋₆ alkyl;

R³ and R⁴ are independently selected from the group consisting of H and C₁₋₆ alkyl; wherein at least one of R³ and R⁴ is not H; or R³ and R⁴ together with the atom to which they are attached combine to form a 3-6 membered cycloalkyl; and

A is an optionally substituted 6-membered aryl or an optionally substituted 5-6 membered heteroaryl;

with the proviso that when

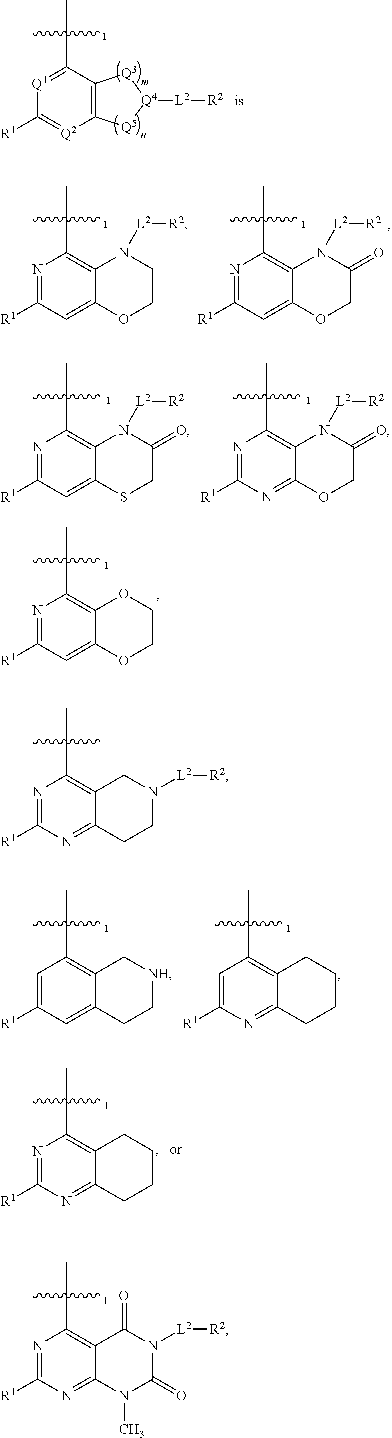

then R¹ is not H.

The present disclosure provides for compounds of Formula (IV-a), (IV-b), (IV-c), (IV-d), or (IV-e),

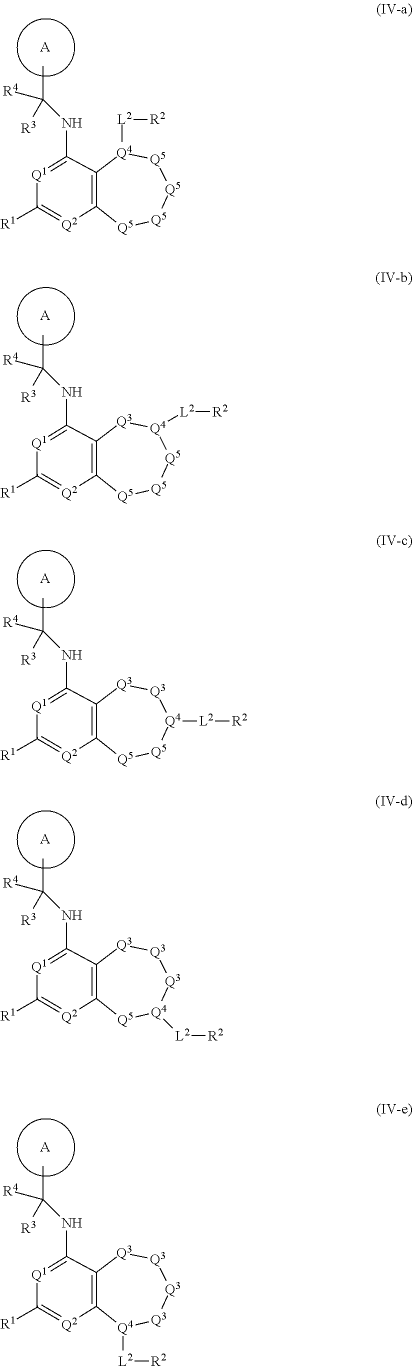

and pharmaceutically acceptable salts, prodrugs, solvates, hydrates, tautomers, or isomers thereof, wherein:

Q¹ and Q² are independently CH or N;

each Q³ and Q⁵ are independently C(R^{QC})₂, NR^{QN}, CO, O, or SO₂, wherein each R^{QC} is independently H, F, Cl, Br, or aryl, and wherein each R^{QN} is independently H, C₁₋₆ alkyl, or aryl (e.g., 6-10 membered aryl);

Q⁴ is CH or N;

wherein at least one of Q¹, Q², Q³, Q⁴, and Q⁵ is N, NR^{QN}, O, or SO₂;

R¹ is H, halogen, C₁₋₆ alkyl, cyclopropyl, —CN, or —OR^{1a}; wherein R^{1a} is H or C₁₋₆ alkyl;

L² is a bond, —C(O)—, —C(O)O—, —C(O)NH(CH₂)ₒ—, —S(O)₂—, —C(O)(CH₂)ₚ—, —(CH₂)ₚ—, or —O—; wherein o is 0, 1, or 2; and wherein p is a number from 1 to 6;

R² is H, —(CH₂)_{q}CH₃, cycloalkyl (e.g., 3-14 membered cycloalkyl), cycloalkenyl (e.g., 3-14 membered cycloalkenyl), heterocyclyl (e.g., 3-14 membered heterocyclyl), aryl (e.g., 6-10 membered aryl), heteroaryl (e.g., 5-10 membered heteroaryl); wherein q is a number from 1 to 5; wherein each cycloalkyl, cycloalkenyl, heterocyclyl, aryl, or heteroaryl is optionally substituted with one or more C₁₋₆ alkyl, —OH, halogen, —C(O)R^{2a} or —C(O)NR^{2b}R^{2c}; wherein R^{2a} is C₁₋₆ alkyl or —(CH₂)ᵣOCH₃, wherein r is 1, 2, or 3; wherein R^{2b} is H or C₁₋₆ alkyl; and wherein R^{2c} is H or C₁₋₆ alkyl;

R³ and R⁴ are independently selected from the group consisting of H and C₁₋₆ alkyl; wherein at least one of R³ and R⁴ is not H; or R³ and R⁴ together with the atom to which they are attached combine to form a 3-6 membered cycloalkyl; and

A is an optionally substituted 6-membered aryl or an optionally substituted 5-6 membered heteroaryl;

with the proviso that when

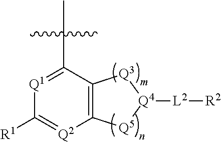

is

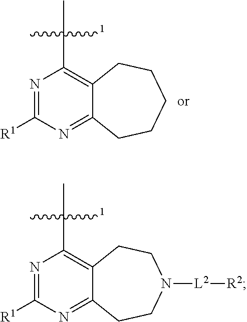

then R¹ is not H.

As described herein for Formula (I)-(IV), A is an optionally substituted 6-membered aryl or an optionally substituted 5-6 membered heteroaryl.

In certain embodiments of Formula (I)-(IV), A is an optionally substituted 6-membered aryl. In certain embodiments, A is an optionally substituted 5-6-membered heteroaryl. In certain embodiments, A is an optionally substituted 5-membered heteroaryl. In certain embodiments, A is an optionally substituted 6-membered heteroaryl.

In certain embodiments of Formula (I)-(IV), A is an optionally substituted 6-membered aryl, wherein the substituents form a fused ring, i.e., the A group is a bicyclic group. In certain embodiments, the A group is a fused bicyclic group containing 18 ring atoms or fewer, 14 ring atoms or fewer, or 10 ring atoms of fewer. The fused ring may be a 3-8 membered cycloalkyl, a 4-8 membered cycloalkenyl, a 3-14 membered heterocyclyl, or a 3-8 membered heteroaryl. In some embodiments, the bicyclic ring is optionally substituted with one to three substituents.

In certain embodiments of Formula (I)-(IV), A is a 6-membered aryl. In certain embodiments of Formula I, A is a 6-membered aryl, which is substituted with R⁵, R⁶, R⁷, R⁸, and R⁹, as described herein and shown below:

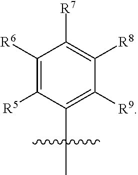

In some embodiments, R⁵, R⁶, R⁷, R⁸, and R⁹ are independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OH, halogen, —NO₂, —CN, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, halogen, —NO₂, oxo, —CN, —R¹⁰, —OR, —NR¹¹R¹², —SR, —S(O)₂NR¹¹R¹², —S(O)₂R, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, 3-8 membered cycloalkyl, 3-14 membered heterocyclyl, 6-10 membered aryl, or 5-10 membered heteroaryl, or any two adjacent R⁵, R⁶, R⁷, R⁸, and R⁹ form a 3-14 membered fused ring.

In some embodiments, R⁵, R⁶, R⁷, R⁸, and R⁹ are independently selected from the group consisting of H, D, C₁₋₆ alkyl, C₂₋₆ alkenyl, 4-8 membered cycloalkenyl, C₂₋₆ alkynyl, 3-8 membered cycloalkyl, —OH, halogen, —NO₂, —CN, —NR¹¹R¹², —SR¹⁰, —S(O)₂NR¹¹R¹², —S(O)₂R, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, and 3-8 membered cycloalkyl are independently optionally substituted with —OH, halogen, —NO₂, oxo, —CN, —R¹⁰, —OR¹⁰, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, 3-14 membered heterocyclyl, 6-10 membered aryl, or 5-10 membered heteroaryl.

In the above, R¹⁰, R¹, and R¹² are at each occurrence independently selected from H, D, C₁₋₆ alkyl, C₂₆ alkenyl, 4-8 membered cycloalkenyl, C₂₆ alkynyl, 3-8 membered cycloalkyl, 3-14 membered heterocyclyl, —OR¹³, —SR¹³, halogen, —NR¹³R¹⁴, —NO₂, and —CN.

In the above, R¹³ and R¹⁴ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN.

In certain embodiments of Formula (I)-(IV), A is a 5-6 membered heteroaryl.

In certain embodiments of Formula I, A is a 5-membered heteroaryl, which is substituted with R⁶ and R⁷, as described herein and shown below:

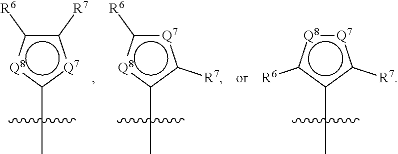

In some embodiments, Q⁷ and Q⁸ are independently CH, N, NH, 0, or S, provided at least one of Q⁷ and Q⁸ is N, NH, 0, or S.

In some embodiments, R⁶ and R⁷ are independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OH, halogen, —NO₂, —CN, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, halogen, —NO₂, oxo, —CN, —R¹⁰, —OR¹⁰, —NR¹¹R¹², —SR¹⁰, —S(O)₂NR¹¹R¹², —S(O)₂R, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, 3-8 membered cycloalkyl, 3-14 membered heterocyclyl, 6-10 membered aryl, or 5-10 membered heteroaryl.

In some embodiments, R⁶ and R⁷ are independently selected from the group consisting of H, D, C₁₋₆ alkyl, C₂₋₆ alkenyl, 4-8 membered cycloalkenyl, C₂₋₆ alkynyl, 3-8 membered cycloalkyl, —OH, halogen, —NO₂, —CN, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, and 3-8 membered cycloalkyl are independently optionally substituted with —OH, halogen, —NO₂, oxo, —CN, —R¹⁰, —OR¹⁰, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, 3-14 membered heterocyclyl, 6-10 membered aryl, or 5-10 membered heteroaryl.

In the above, R¹⁰, R¹¹, and R¹² are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR 3, —SR¹³, halogen, —NR¹³R¹⁴, —NO₂, or —CN.

In the above, R¹³ and R¹⁴ are at each occurrence independently selected from H, D, C₁₋₆ alkyl, C₂₋₆ alkenyl, 4-8 membered cycloalkenyl, C₂₋₆ alkynyl, 3-8 membered cycloalkyl, or 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN.

In certain embodiments of Formula (I)-(IV), A is a 6-membered heteroaryl. In certain embodiments of Formula I, A is a 6-membered heteroaryl, which is substituted with R⁵, R⁶, R⁷, R⁸, and R⁹, as described herein and shown below:

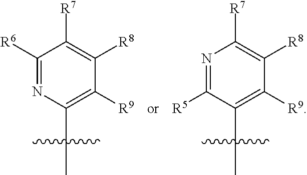

In some embodiments, R⁵, R⁶, R⁷, R⁸, and R⁹ are independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OH, halogen, —NO₂, —CN, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, halogen, —NO₂, oxo, —CN, —R¹⁰, —OR¹⁰, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, 3-8 membered cycloalkyl, 3-14 membered heterocyclyl, 6-10 membered aryl, or 5-10 membered heteroaryl, or any two adjacent R, R⁸, and R⁹ form a 3-14 membered fused ring.

In some embodiments, R⁵, R⁶, R⁷, R⁸, and R⁹ are independently selected from the group consisting of H, D, C₁₋₆ alkyl, C₂₋₆ alkenyl, 4-8 membered cycloalkenyl, C₂₋₆ alkynyl, 3-8 membered cycloalkyl, —OH, halogen, —NO₂, —CN, —NR¹¹R¹², —SR¹⁰, —S(O)₂NR¹¹R¹², —S(O)₂R, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, and 3-8 membered cycloalkyl are independently optionally substituted with —OH, halogen, —NO₂, oxo, —CN, —R¹⁰, —OR¹⁰, —NR¹¹R¹², —SR¹⁰, —S(O)₂NR¹¹R¹², —S(O)₂R, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, 3-14 membered heterocyclyl, 6-10 membered aryl, or 5-10 membered heteroaryl.

In the above, R¹⁰, R¹¹, and R¹² are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR 3, —SR¹³, halogen, —NR¹³R¹⁴, —NO₂, and —CN.

In the above, R¹³ and R¹⁴ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN.

The present disclosure provides for compounds of Formula (V),

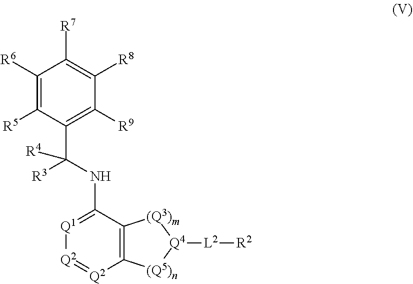

or a pharmaceutically acceptable salt, solvate, isomer, prodrug, or tautomer thereof, wherein:

L², Q¹, Q², Q³, Q⁴, Q⁵, m, n, R¹, R², R³ and R⁴ are as defined in Formula (I);

R⁵, R⁶, R⁷, R⁸, and R⁹ are independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OH, halogen, —NO₂, —CN, —NR¹¹R¹², —SR, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, halogen, —NO₂, oxo, —CN, —R¹⁰, —OR¹⁰, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, 3-8 membered cycloalkyl, 3-14 membered heterocyclyl, 6-10 membered aryl, or 5-10 membered heteroaryl, or any two adjacent R⁵, R⁶, R⁷, R⁸, and R⁹ form a 3-14 membered fused ring;

R¹⁰, R¹¹, and R¹² are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR¹³, —SR¹³, halogen, —NR¹³R¹⁴, —NO₂, and —CN; and R¹³ and R¹⁴ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN.

The present disclosure provides for compounds of Formula (V-a),

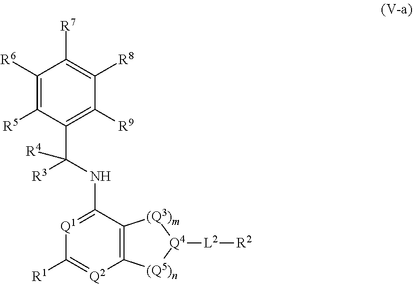

or a pharmaceutically acceptable salt, solvate, isomer, prodrug, or tautomer thereof, wherein:

Q¹, Q³, Q⁴, Q⁵, m, n, R², R³, R⁴, R, R⁶, R⁷, R⁸, R⁹, R¹⁰, R¹¹, R¹², R¹³ and R¹⁴ are as defined in Formula (V);

Q² is CH or N;

wherein at least one of Q¹, Q², Q³, Q⁴, and Q⁵ is N, NR^{QN}, O, or SO₂;

R¹ is selected from the group consisting of H, halogen, C₁₋₆ alkyl, cyclopropyl, —CN, and —OR^{1a}; wherein R^{1a} is H or C₁₋₆ alkyl; and

L² is selected from the group consisting of a bond, —C(O)—, —C(O)O—, —C(O)NH(CH₂)ₒ—, —S(O)₂—, —C(O)(CH₂)ₚ—, —(CH₂)ₚ—, or —O—; wherein o is 0, 1, or 2; and wherein p is a number from 1 to 6.

The present disclosure provides for compounds of Formula (V-b),

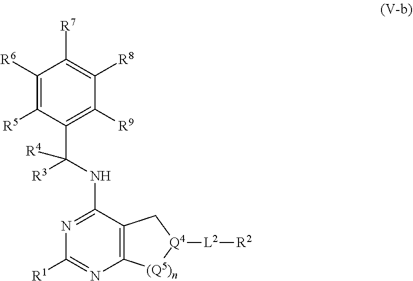

or a pharmaceutically acceptable salt, solvate, isomer, prodrug, or tautomer thereof, wherein:

Q⁴ is CH, C, or N;

each Q⁵ is independent CH₂, N—CH₃, or CO, and n is 1 or 2;

L² is selected from the group consisting of a bond, —C(O)—, —S(O)₂—, —C(O)NH(CH₂)ₒ—, —C(O)(CH₂)ₚ—, —(CH₂)ₚ—; wherein o is 0, 1, or 2; and wherein p is a number from 1 to 6;

R¹ is selected from the group consisting of H, C₁₋₆ alkyl, halogen, —CONHR^{1a}, —NHR^{1a}, —OR^{1a}, and azetidinyl; wherein each C₁₋₆ alkyl and azetidinyl is optionally substituted with halogen, R^{1a}, —NHR^{1a}, or —OR^{1a}; wherein R^{1a} is H, C₁₋₆ alkyl, cyclopropyl, 3-6 membered heterocyclyl, or C1.6 haloalkyl;

R² is selected from the group consisting of H, C₁₋₆ alkyl, —NR^{2b}R^{2c}, —OR^{2a}, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl are independently optionally substituted with C₁₋₆ alkyl, C₁₋₆ haloalkyl, C₁₋₆ hydroxyalkyl, C₁₋₆ methoxyalkyl, —OH, —OR^{2a}, oxo, ═N, halogen, —C(O)R^{2a}, —C(O)OR^{2a}, —C(O)NR^{2b}R^{2c}, —SO₂R^{2a}, —CN, —NR^{2b}R^{2c}, 3-6 membered cycloalkyl, 3-7 membered heterocyclyl, 6-10 membered aryl, or 5-10 membered heteroaryl;
wherein R^{2a} is H, C₁₋₆ alkyl, C₁₋₆ haloalkyl, 3-7 membered heterocyclyl, or —(CH₂)ᵣOCH₃, wherein r is 1, 2, or 3;
wherein R^{2b} is H or C₁₋₆ alkyl;
wherein R^{2c} is H or C₁₋₆ alkyl;

R³ and R⁴ are each independently selected from the group consisting of —H, —CH₃, and —CH₂CH₃;

R⁵, R⁶, R⁷, R⁸, and R⁹ are independently selected from the group consisting of H, C₁₋₆ alkyl, 4-8 membered cycloalkenyl, 3-8 membered cycloalkyl, 3-14 membered heterocyclyl, halogen, and —NR¹¹R¹², wherein each C₁₋₆ alkyl, 4-8 membered cycloalkenyl, 3-8 membered cycloalkyl, and 3-14 membered heterocyclyl are independently optionally substituted with —OH, halogen, —CN, —R¹⁰, —OR¹⁰, —NR¹¹R¹², 3-8 membered cycloalkyl, or 3-14 membered heterocyclyl; or any two adjacent R⁵, R⁶, R⁷, R⁸, and R⁹ forms a 4-8 membered cycloalkenyl fused ring, a 3-8 membered cycloalkyl fused ring, or a 3-14 membered heterocyclyl fused ring, wherein the 4-8 membered cycloalkenyl fused ring, the 3-8 membered cycloalkyl fused ring, or the 3-14 membered heterocyclyl fused ring are optionally substituted with —F or —CH₂OH; and

R¹⁰, R¹¹, and R¹² are at each occurrence independently selected from H, C₁₋₆ alkyl, 3-8 membered cycloalkyl, or 3-14 membered heterocyclyl.

In some embodiments of the structure (V-b), R¹ is selected from the group consisting of —H, —CH₃, —Cl, —OH, —CH₂F, —CF₂CH₂NH₂, —CF₂CH₂OH, —CONH₂,

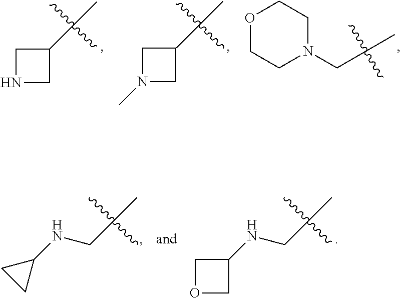

In some embodiments of the structure (V-b), R⁵, R⁶, R⁷, R⁸, and R⁹ are independently selected from the group consisting of —H, —CF₃, —NH₂, —F, —Br, —CHF₂, —CH₂F, —CH₃, —CF₂CH₂OH, —CF₂CH₂NH₂, —CF₂CH₂OCH₃, —CHFCH₂OH, —CF₂C(CH₃)₂OH, —CH₂CH₂OH, —CH(CH₂)CH₂OH, —C(CH₃)₂CN,

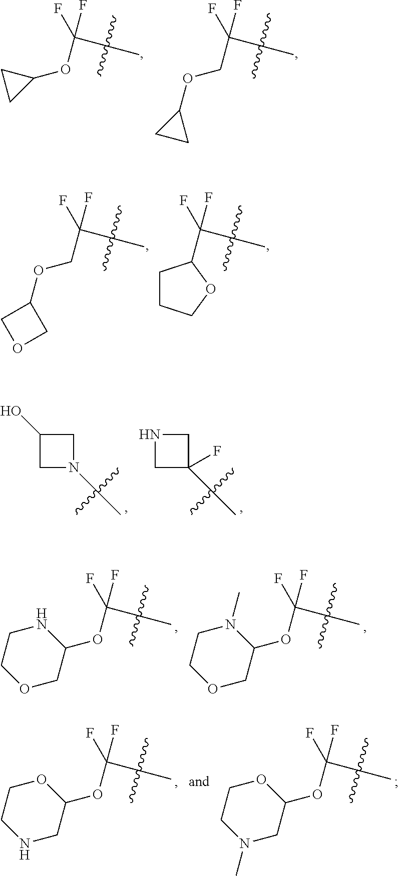

or any two adjacent R⁵, R⁶, R⁷, R⁸, and R⁹ forms the 4-8 membered cycloalkenyl fused ring, 3-8 membered cycloalkyl fused ring, or 3-14 membered heterocyclyl fused ring, wherein the 4-8 membered cycloalkenyl fused ring, the 3-8 membered cycloalkyl fused ring, or the 3-14 membered heterocyclyl fused ring are optionally substituted with —F or —CH₂OH.

The present disclosure provides for compounds of Formula (VI),

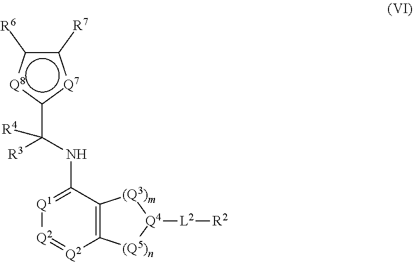

or a pharmaceutically acceptable salt, solvate, isomer, prodrug, or tautomer thereof, wherein:

L², Q¹, Q², Q³, Q⁴, Q⁵, m, n, R¹, R², R³ and R⁴ are as defined in Formula (I);

Q⁷ and Q⁸ are each independently CH, N, NH, O, or S, provided at least one of Q⁷ and Q⁸ is N, NH, O, or S;

R⁶ and R⁷ are independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OH, halogen, —NO₂, —CN, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, halogen, —NO₂, oxo, —CN, —R¹⁰, —OR¹⁰, —NR¹¹R¹², —SR, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, 3-8 membered cycloalkyl, 3-14 membered heterocyclyl, 6-10 membered aryl, or 5-10 membered heteroaryl,

R¹⁰, R¹¹, and R¹² are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR¹³, —SR¹¹, halogen, —NR¹³R¹⁴—NO₂, and —CN; and

R¹³ and R¹⁴ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN.

The present disclosure provides for compounds of Formula (VI-a),

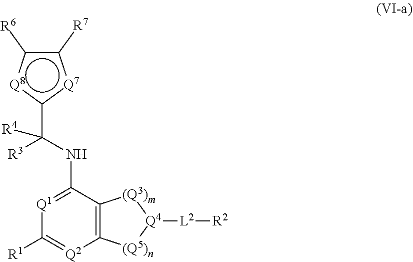

and pharmaceutically acceptable salts, prodrugs, solvates, hydrates, tautomers, or isomers thereof, wherein Q¹, Q², Q³, Q⁵, Q⁷, Q⁸, R¹, R², R³, R⁴, R⁶, R⁷, L², m, and n are described as above.

The present disclosure provides for compounds of Formula (VII-a) and (VII-b),

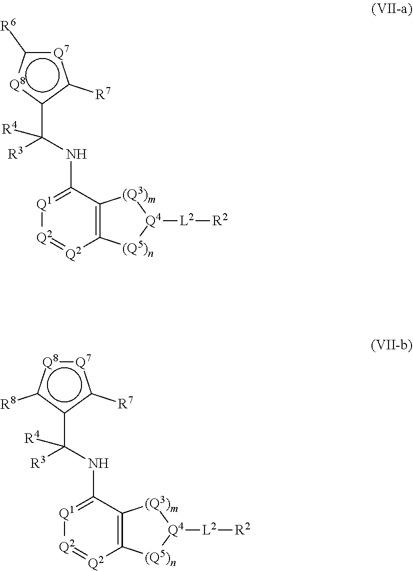

or a pharmaceutically acceptable salt, solvate, isomer, prodrug, or tautomer thereof, wherein:

L², Q¹, Q², Q³, Q⁴, Q⁵, m, n, R¹, R², R³ and R⁴ are as defined in Formula (I);

Q⁷ and Q⁸ are each independently CH, N, NH, O, or S, provided at least one of Q⁷ and Q⁸ is N, NH, O, or S;

R⁶ and R⁷ are independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OH, halogen, —NO₂, —CN, —NR¹¹R¹², —SR¹⁰, —S(O)₂NR¹¹R¹², —S(O)₂R¹⁰, —NR¹⁰S(O)₂NR¹¹R¹². —NR¹⁰S(O)₂R¹¹, —S(O)NR¹¹R¹², —S(O)R¹⁰, —NR¹⁰S(O)NR¹¹R¹², —NR¹⁰S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, halogen, —NO₂, oxo, —CN, —R¹⁰, —OR¹⁰, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, 3-8 membered cycloalkyl, 3-14 membered heterocyclyl, 6-10 membered aryl, or 5-10 membered heteroaryl,

R¹⁰, R¹¹, and R¹² are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR¹³, —SR¹³, halogen, —NR¹³R¹⁴, —NO₂, and —CN; and

R¹³ and R¹⁴ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN.

The present disclosure provides for compounds of Formula (VII-c) and (VII-d),

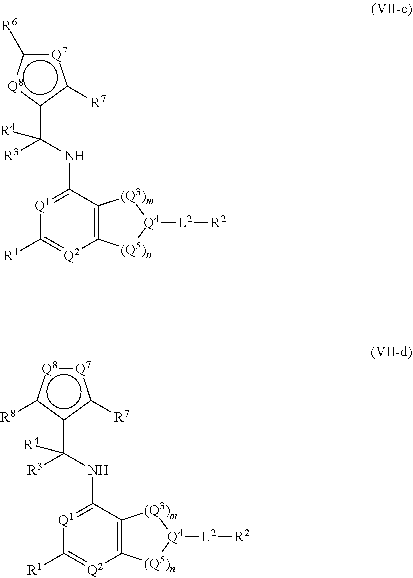

and pharmaceutically acceptable salts, prodrugs, solvates, hydrates, tautomers, or isomers thereof, wherein Q¹, Q², Q³, Q⁵, Q⁷, Q⁸, R¹, R², R³, R⁴, R⁶, R⁷, L², m, and n are described as above.

The present disclosure provides for compounds of Formula (VIII-a) and (VIII-b),

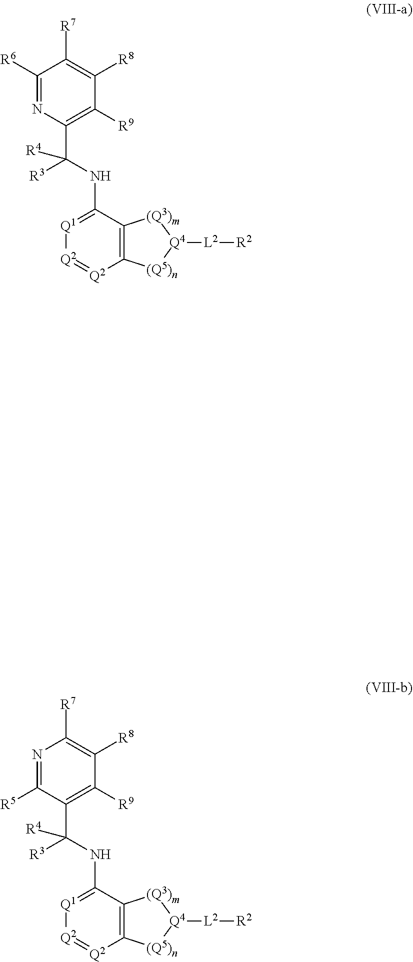

or a pharmaceutically acceptable salt, solvate, isomer, prodrug, or tautomer thereof, wherein:

L², Q¹, Q², Q³, Q⁴, Q⁵, m, n, R¹, R², R³ and R⁴ are as defined in Formula (I);

R⁵, R⁶, R⁷, R⁸, and R⁹ are independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OH, halogen, —NO₂, —CN, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, halogen, —NO₂, oxo, —CN, —R¹⁰, —OR¹⁰, —NR¹¹R¹², —SR¹⁰, —S(O)₂NR¹¹R¹², —S(O)₂R, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, 3-8 membered cycloalkyl, 3-14 membered heterocyclyl, 6-10 membered aryl, or 5-10 membered heteroaryl, or any two adjacent R⁷, R⁸, and R⁹ form a 3-14 membered fused ring;

R¹⁰, R¹¹, and R¹² are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR¹³, —SR¹³, halogen, —NR¹³R¹⁴. —NO₂, and —CN; and

R¹³ and R¹⁴ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN.

The present disclosure provides for compounds of Formula (VIII-c) and (VIII-d),

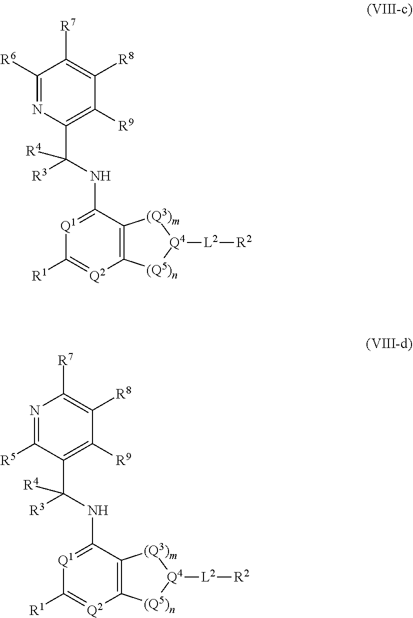

and pharmaceutically acceptable salts, prodrugs, solvates, hydrates, tautomers, or isomers thereof, wherein Q¹, Q², Q³, Q⁵, R¹, R², R³, R⁴, R⁵, R⁶, R⁷, L², m, and n are described as above.

As described above, m is 0, 1, 2, or 3; n is 0, 1, 2, or 3; wherein when m is 0, then n is not 0. In certain embodiments, m is 0. In certain embodiments, m is 1. In certain embodiments, m is 2. In certain embodiments, m is 3. In certain embodiments, n is 0. In certain embodiments, n is 1. In certain embodiments, n is 2. In certain embodiments, n is 3.

In certain embodiments, m is 1 and n is 1. In certain embodiments, m is 1 and n is 2. In certain embodiments, m is 2 and n is 1. In certain embodiments, m is 1 and n is 3. In certain embodiments, m is 2 and n is 2.

As described above, Q¹ and Q² are independently CH or N. In certain embodiments, Q¹ is CH. In certain embodiments, Q¹ is N. In certain embodiments, Q² is CH. In certain embodiments, Q² is N.

As described above, Q⁴ is C or N. In certain embodiments, Q⁴ is C. In certain embodiments, Q⁴ is N.

As described above, each Q³ and Q⁵ are independently C(R^{QC})₂, NR^{QN}, CO, O, or SO₂, wherein each R^{QC} is independently H, F, Cl, Br, or aryl (e.g., 6-10 membered aryl), and wherein each R^{QN} is independently H, C₁₋₆ alkyl, or aryl (e.g., 6-10 membered aryl). In certain embodiments, each Q³ and Q⁵ are independently C(R^{QC})₂, NR^{QN}, O, or SO₂, wherein each R^{QC} is independently H, F, Cl, Br, or aryl (e.g., 6-10 membered aryl), and wherein each R^{QN} is independently H, C₁₋₆ alkyl, or aryl (e.g., 6-10 membered aryl). In certain embodiments, each Q³ and Q⁵ are independently C(R^{QC})₂ or NR^{QN}, wherein each R^{QC} is independently H, F, Cl, Br, or aryl (e.g., 6-10 membered aryl), and wherein each R^{QN} is independently H, C₁₋₆ alkyl, or aryl (e.g., 6-10 membered aryl). In certain embodiments, each Q³ and Q⁵ are independently CH₂ or NH. In certain embodiments, each Q³ and Q⁵ are independently CH₂.

In some embodiments,

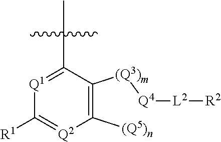

is selected from the group consisting of

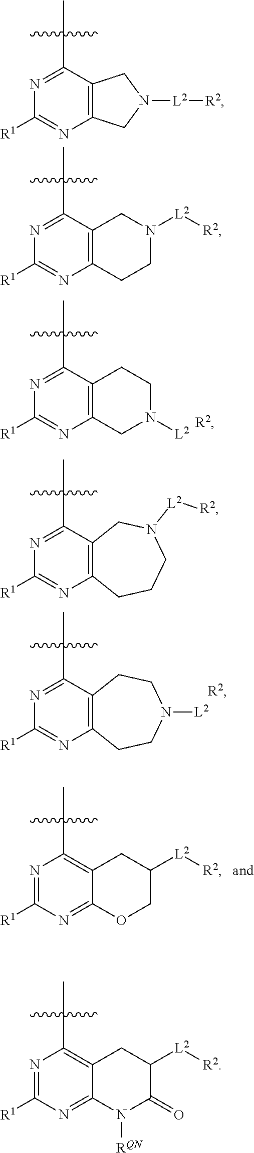

In some embodiments,

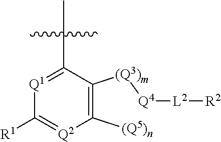

is

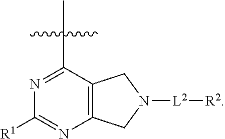

In some embodiments,

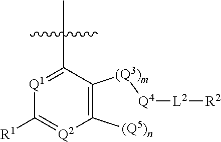

is

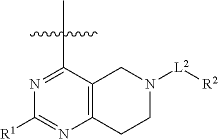

In some embodiments,

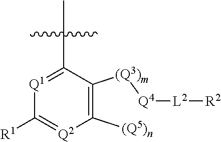

is

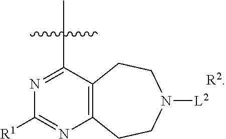

In certain embodiments,

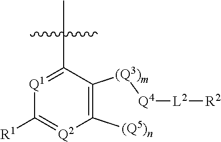

is selected from the group consisting of

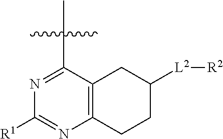

and

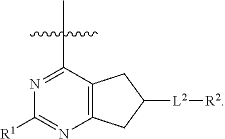

In some embodiments, R¹ is selected from the group consisting of H, C₁₋₆ alkyl, halogen, —CONHR^{1a}, —NHR^{1a}, —OR^{1a}, cyclopropyl, azetidinyl, and —CN; wherein each C₁₋₆ alkyl and azetidinyl is optionally substituted with halogen, R^{1a}, —NHR^{1a}, or —OR^{1a}; wherein Ria is H, C₁₋₆ alkyl, cyclopropyl, 3-6 membered heterocyclyl, or C₁₋₆ haloalkyl

In some embodiments, R¹ is selected from the group consisting of H, C₁₋₆ alkyl, halogen, —NHR^{1a}, —OR^{1a}, azetidinyl, cyclopropyl, and —CN; wherein each C₁₋₆ alkyl and azetidinyl is optionally substituted with halogen, —R^{1a}, —NHR^{1a}, or —OR^{1a}; wherein R^{1a} is H, C₁₋₆ alkyl, 3-6 membered heterocyclyl, or C₁₋₆ haloalkyl.

In some embodiments, R¹ is H, halogen, C₁₋₆ alkyl, cyclopropyl, —CN, or —OR^{1a}; wherein R^{1a} is H or C₁₋₆ alkyl. In certain embodiments, R¹ is halogen, C₁₋₆ alkyl, cyclopropyl, —CN, or —OR^{1a}; wherein R^{1a} is H or C₁₋₆ alkyl.

In certain embodiments, R¹ is H. In certain embodiments, R¹ is halogen. In certain embodiments, R¹ is C₁₋₆ alkyl. In certain embodiments, R¹ is C₁ alkyl, C₂ alkyl, C₃ alkyl, C₄ alkyl, C₅ alkyl, or C₆ alkyl. In some embodiments, the C₁₋₆ alkyl is substituted. In certain embodiments, R¹ is cyclopropyl. In certain embodiments, R¹ is —CN. In certain embodiments, R¹ is —OR^{1a}; wherein R^{1a} is H or C₁₋₆ alkyl. In certain embodiments, R¹ is —OH. In certain embodiments, R¹ is —OR^{1a}; wherein R^{1a} is C₁₋₆ alkyl.

In certain embodiments, R¹ is selected from the group consisting of H, —CH₃, —CH₂OH, —CH₂NH₂, —CH₂CH₃, —CF₂CH₂OH, —CONH₂, —Cl, —Br, —I, cyclopropyl, —OH, —CN, —OCH₃, —OCH₂CH₃, —NHCH₃, —CHF₂, —CF₃, —OCF₃,

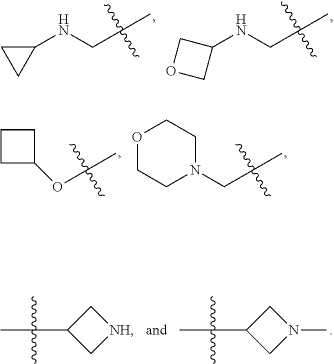

In certain embodiments, R¹ is selected from the group consisting of H, —CH₃, —CH₂OH, —CH₂NH₂, —CH₂CH₃, —Cl, —Br, —I, cyclopropyl, —OH, —CN, —OCH₃, —OCH₂CH₃, —NHCH₃, —CHF₂, —CF₃, —OCF₃, and

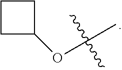 [SMILES: *OC1CCC1]

In some embodiments, L² is selected from the group consisting of a bond, —C(O)—, —C(O)O—, —C(O)NH(CH₂)ₒ—, —S(O)₂—,

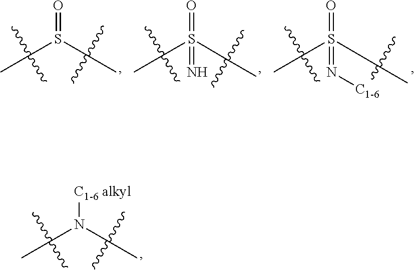

—C(O)(CH₂)ₚ—, —(CH₂)ₚ—, and —O—; wherein o is 0, 1, or 2; and wherein p is a number from 1 to 6. In some embodiments, wherein L² comprises a carbonyl group, the carbon of the carbonyl group is bonded to Q⁴.

In some embodiments, L² is selected from the group consisting of a bond, —C(O)—, —C(O)O—, —C(O)NH(CH₂)ₒ—, —S(O)₂—, —C(O)(CH₂)ₚ—, —(CH₂)ₚ—, and —O—; wherein o is 0, 1, or 2; and wherein p is a number from 1 to 6. In some embodiments, wherein L² comprises a carbonyl group, the carbon of the carbonyl group is bonded to Q⁴.

In some embodiments, L² is selected from the group consisting of

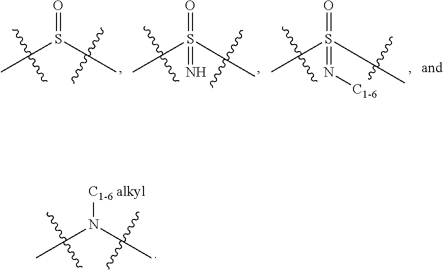

In certain embodiments, L² is a bond. In certain embodiments, L² is —C(O)—. In certain embodiments, L² is —C(O)O—, wherein the carbonyl carbon is bonded to Q⁷. In certain embodiments, L² is —C(O)NH(CH₂)ₒ—, wherein the carbonyl carbon is bonded to Q⁷. In certain embodiments, L² is —S(O)₂—. In certain embodiments, L² is —C(O)(CH₂)ₚ—. In certain embodiments, L² is —(CH₂)ₚ—. In certain embodiments, L² is —O—.

As described herein, o is 0, 1, or 2. In certain embodiments, o is 0. In certain embodiments, o is 1. In certain embodiments, o is 2.

As described above, p is a number from 1 to 6. In certain embodiments, p is 1. In certain embodiments, p is 2. In certain embodiments, p is 3. In certain embodiments, p is 4. In certain embodiments, p is 5. In certain embodiments, p is 6.

In some embodiments, R² is selected from the group consisting of H, C₁₋₆ alkyl, —NR^{2b}R^{2c}, —OR^{2a}, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl are independently optionally substituted with C₁₋₆ alkyl, —OH, —OR^{2a}, oxo, ═N, halogen, —C(O)R^{2a}, —C(OO)R^{2a}, —C(O)NR^{2b}R^{2c}, —CN, —NR^{2b}R^{2c}, 3-6 membered cycloalkyl, 3-7 membered heterocyclyl, 6-10 membered aryl, or 5-10 membered heteroaryl;
wherein R^{2a} is H, C₁₋₆ alkyl, C₁₋₆ haloalkyl, 3-7 membered heterocyclyl, or —(CH₂)ᵣOCH₃, wherein r is 1, 2, or 3;
wherein R^{2b} is H or C₁₋₆ alkyl;
wherein R^{2c} is H or C₁₋₆ alkyl.

In some embodiments, R² is selected from the group consisting of H, —(CH₂)_{q}CH₃, 3-14 membered cycloalkyl, 3-14 membered cycloalkenyl, 3-14 membered heterocyclyl, 6-10 membered aryl, and 5-10 membered heteroaryl; wherein q is a number from 1 to 5; wherein each 3-14 membered cycloalkyl, 3-14 membered cycloalkenyl, 3-14 membered heterocyclyl, 6-10 membered aryl, and 5-10 membered heteroaryl is independently optionally substituted with C₁₋₆ alkyl, —OH, halogen, —C(O)R^{2a}, or —C(O)NR^{2b}R^{2c}; wherein R^{2a} is C₁₋₆ alkyl or —(CH₂)ᵣOCH₃, wherein r is 1, 2, or 3; wherein R^{2b} is H or C₁₋₆ alkyl; and wherein R², is H or C₁₋₆ alkyl.

In certain embodiments, R² is H. In some embodiments, R² is —CH₃. In some embodiments, R² is —CH(CH₃)₂. In certain embodiments, R² is C₁₋₆ alkyl optionally substituted with halogen or —OR²a. In certain embodiments, R² is —(CH₂)_{q}CH₃. In some embodiments, R² is —CH₂CH₂OH. In some embodiments, R² is —CH₂CH₂OCH₂CH₃. In some embodiments, R² is —OCH₃. In certain embodiments, R² is —(CH₂)_{q}CH₃, wherein q is a number from 1 to 5. In certain embodiments, q is 1. In certain embodiments, q is 2. In certain embodiments, q is 3. In certain embodiments, q is 4. In certain embodiments, q is 5. In certain embodiments, R² is C₁₋₆ alkyl optionally substituted with —NR^{2b}R^{2c}. In some embodiments, R² is C₁ alkyl substituted with —NR^{2b}R^{2c} and R^{2b} and R^{2c} are H or —CH₃. In some embodiments, R² is C₁ alkyl substituted with —NR^{2b}R^{2c} and R^{2b} and R^{2c} are both —CH₃.

In certain embodiments, R² is —NR^{2b}R^{2c}, wherein R^{2b} is H or C₁₋₆ alkyl; and wherein R^{2c} is H or C₁₋₆ alkyl. In some embodiments, R² is —NHCH₃. In some embodiments, R² is —(CH₃)₂.

In some embodiments, R² is selected from the group consisting of 3-14 membered cycloalkyl, 3-14 membered cycloalkenyl, 3-14 membered heterocyclyl, 6-10 membered aryl, and 5-10 membered heteroaryl. In some embodiments, wherein R² comprises a cyclic structure, L² is a bond, and the cyclic structure selected from among 3-14 membered cycloalkyl, 3-14 membered cycloalkenyl, 3-14 membered heterocyclyl, 6-10 membered aryl, and 5-10 membered heteroaryl is Spiro bonded to Q⁴. For example,

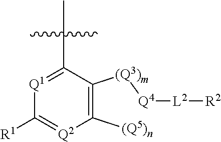

comprises a group having a structure or

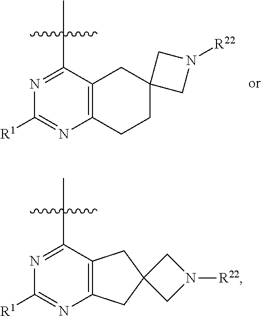

wherein R²² is H, C₁₋₆ alkyl, 3-6 membered cycloalkyl, 3-7 membered heterocyclyl, 6-10 membered aryl, or 5-10 membered heteroaryl.

In certain embodiments, R² is 3-14 membered heterocyclyl, wherein the 3-14 membered heterocyclyl is optionally substituted with C₁₋₆ alkyl optionally substituted with halogen or —OR^{2a}, —OH, —OR²a, oxo, ═N, halogen, —C(O)R^{2a}, —C(OO)R^{2a}, —C(O)NR^{2b}R^{2c}, —CN, —NR^{2b}R^{2c}, 3-6 membered cycloalkyl, 3-7 membered heterocyclyl, 6-10 membered aryl, or 5-10 membered heteroaryl; wherein R^{2a} is H, C₁₋₆ alkyl, C₁₋₆ haloalkyl, 3-7 membered heterocyclyl, or —(CH₂)ᵣOCH₃, wherein r is 1, 2, or 3; wherein R^{2b} is H or C₁₋₆ alkyl; and wherein R^{2c} is H or C₁₋₆ alkyl.

In certain embodiments, R² is 3-14 membered heterocyclyl, wherein the 3-14 membered heterocyclyl is optionally substituted with C₁₋₆ alkyl, —OH, halogen, —C(O)R^{2a}, or —C(O)NR^{2b}R^{2c}; wherein R^{2a} is C₁₋₆ alkyl or —(CH₂)ᵣOCH₃, wherein r is 1, 2, or 3; wherein R^{2b} is H or C₁₋₆ alkyl; and wherein R^{2e} is H or C₁₋₆ alkyl.

In some embodiments, R² is selected from among

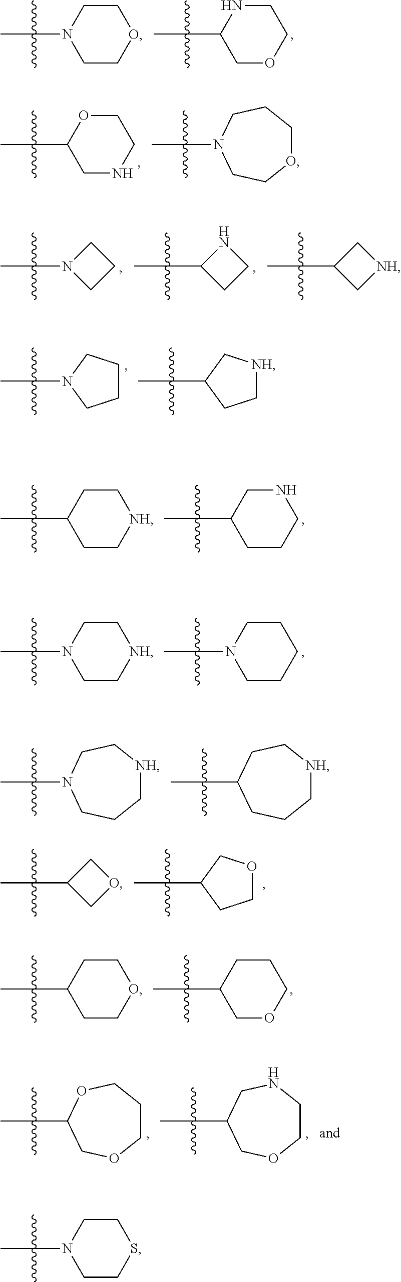

each of which may be optionally substituted at, e.g., any carbon, nitrogen, or sulfur atom.

In some embodiments, R² is selected from among

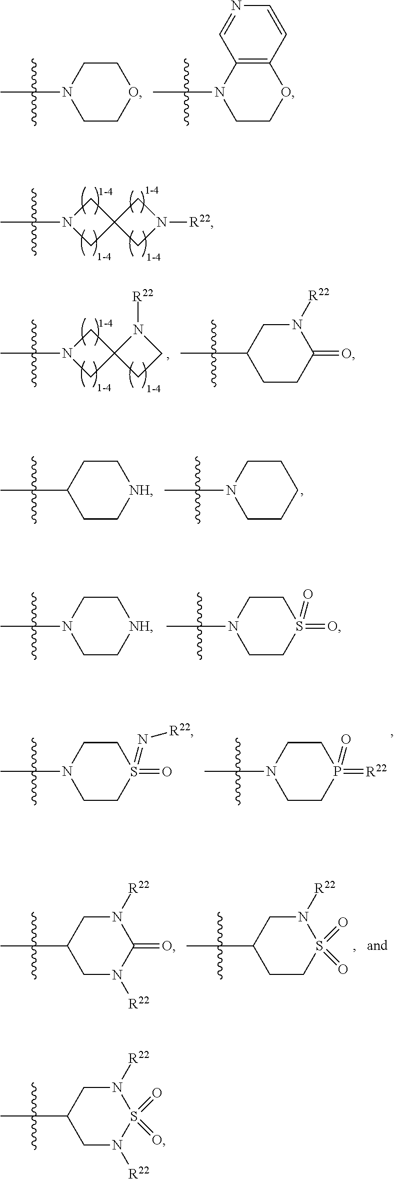

wherein R²² is H, C₁₋₆ alkyl, 3-6 membered cycloalkyl, 3-7 membered heterocyclyl, 6-10 membered aryl, or 5-10 membered heteroaryl.

In some embodiments, R² is selected from among

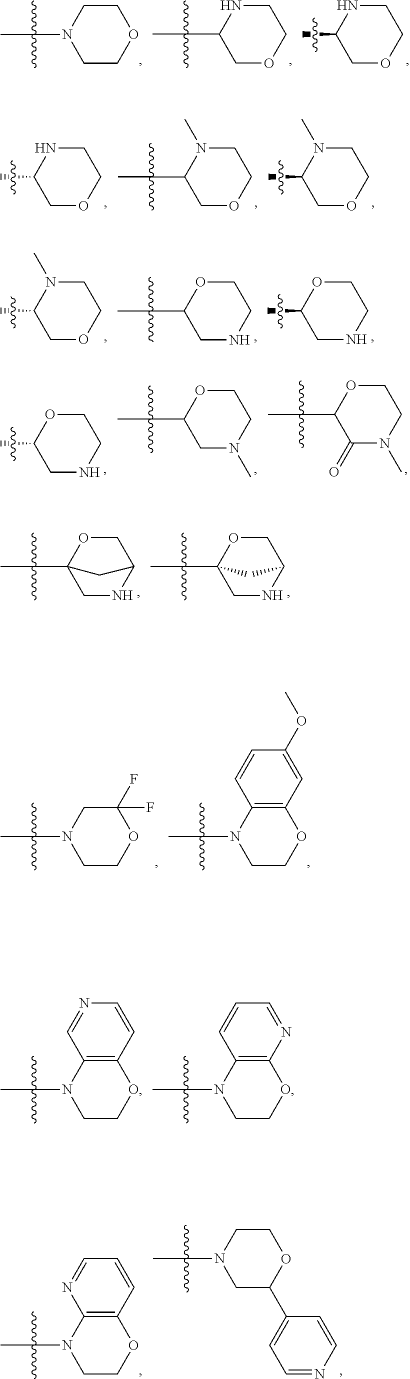

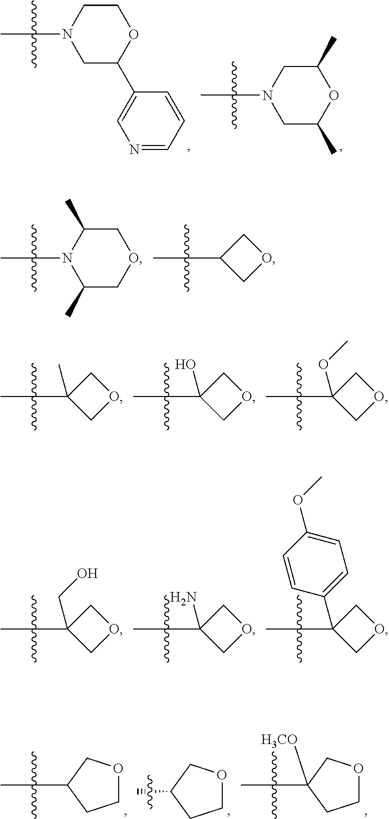

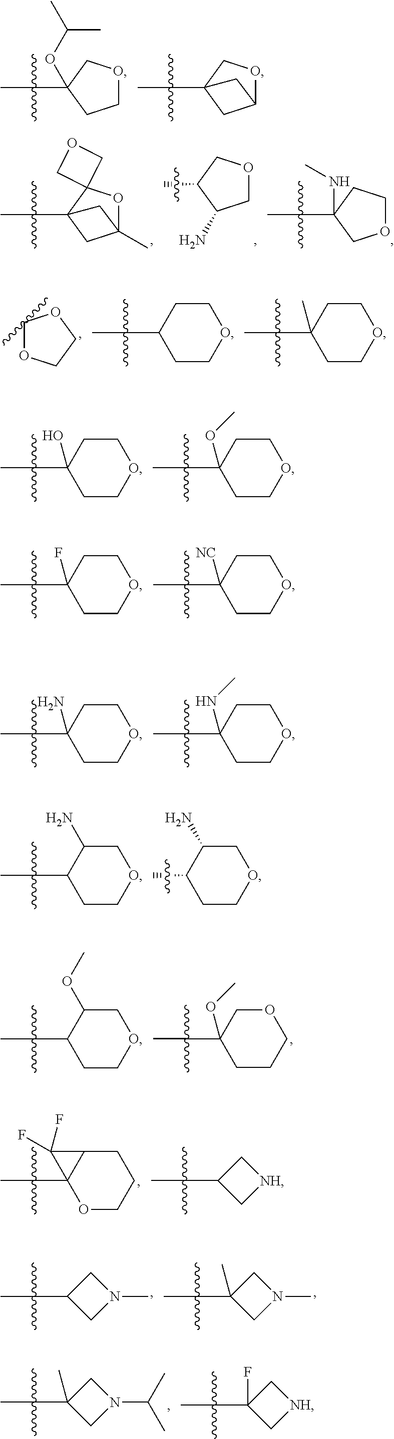

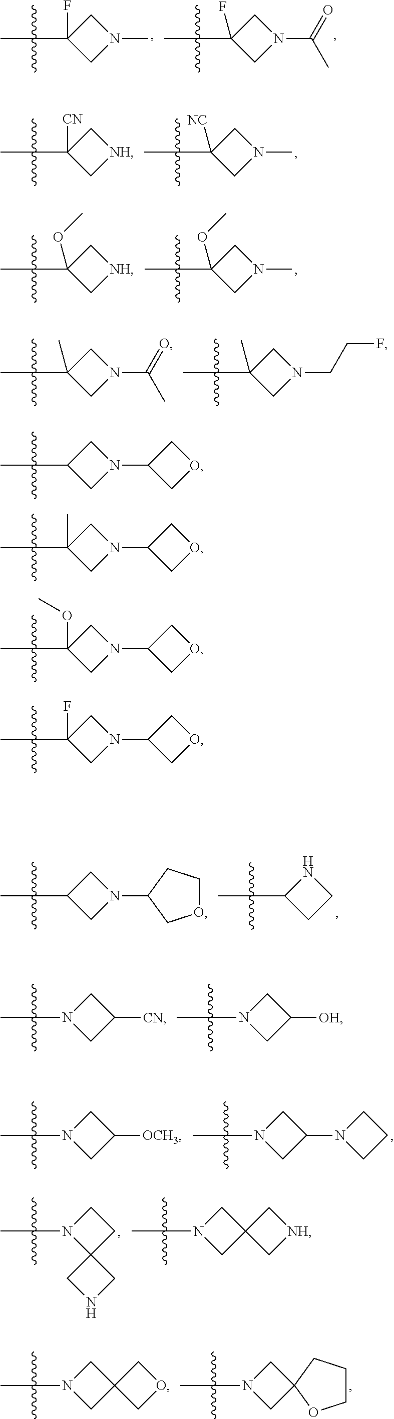

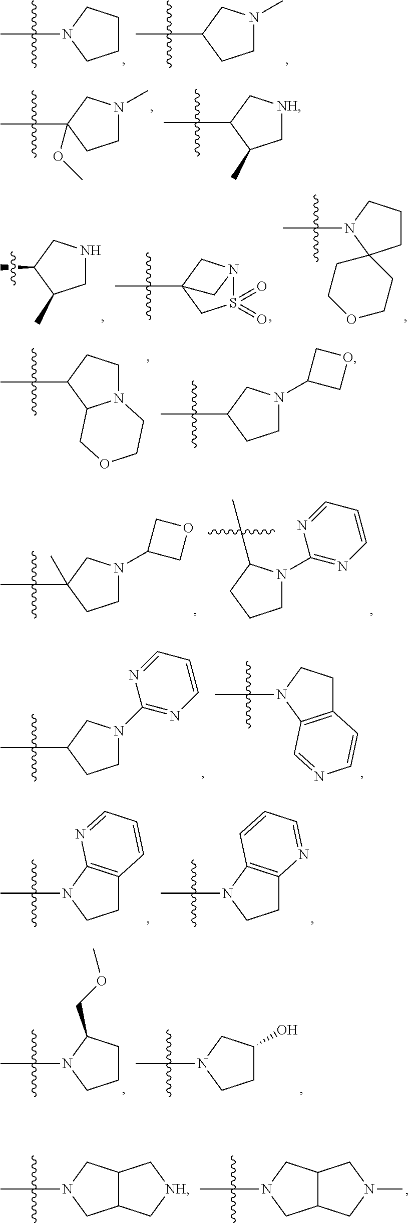

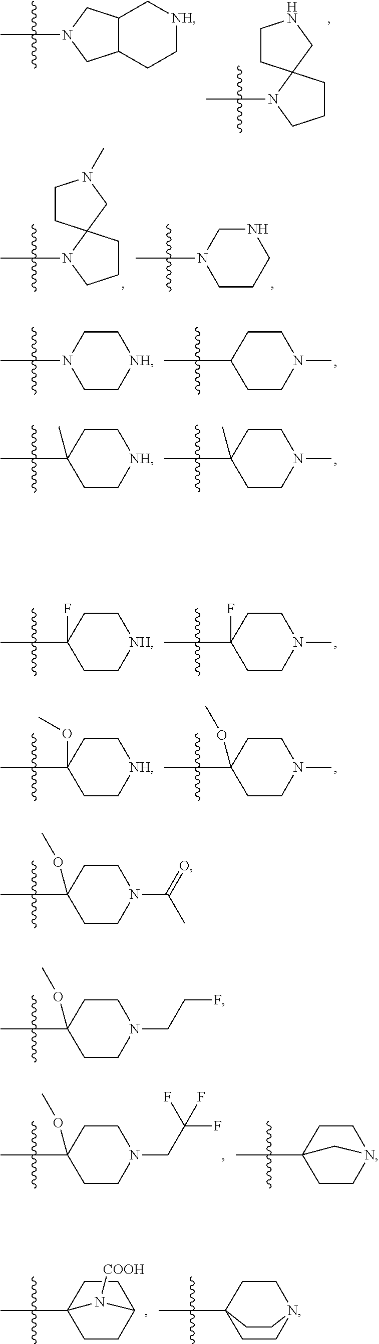

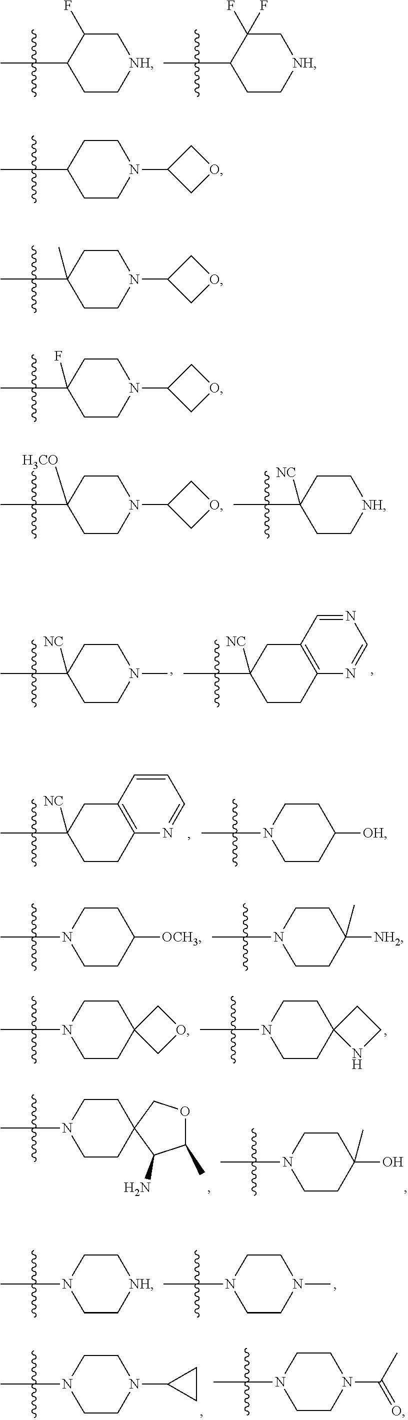

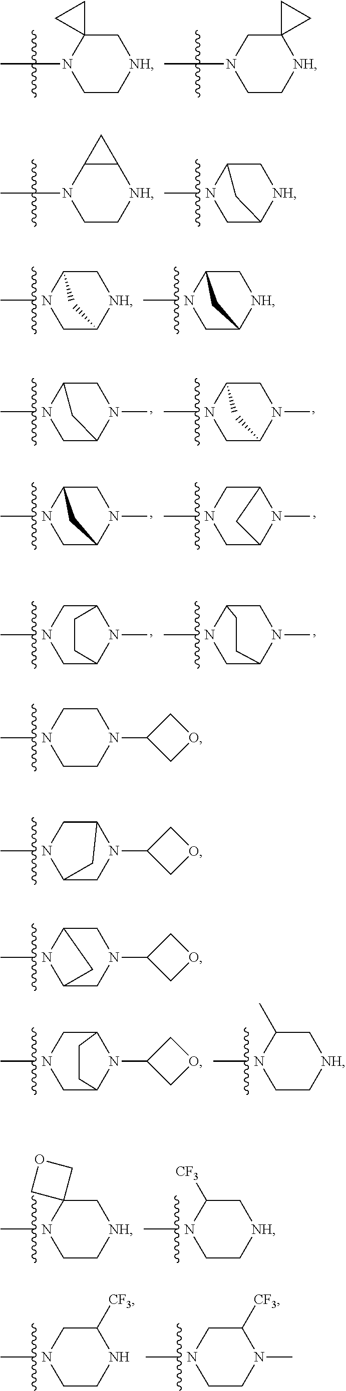

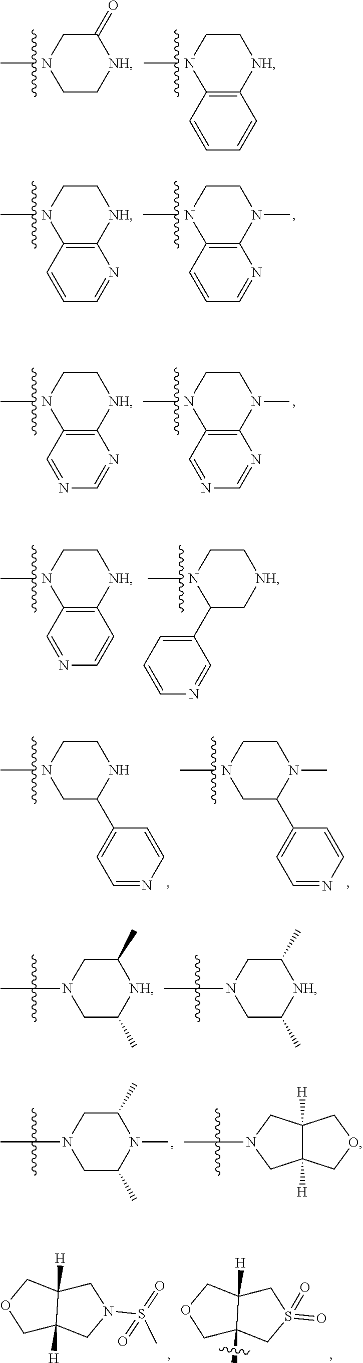

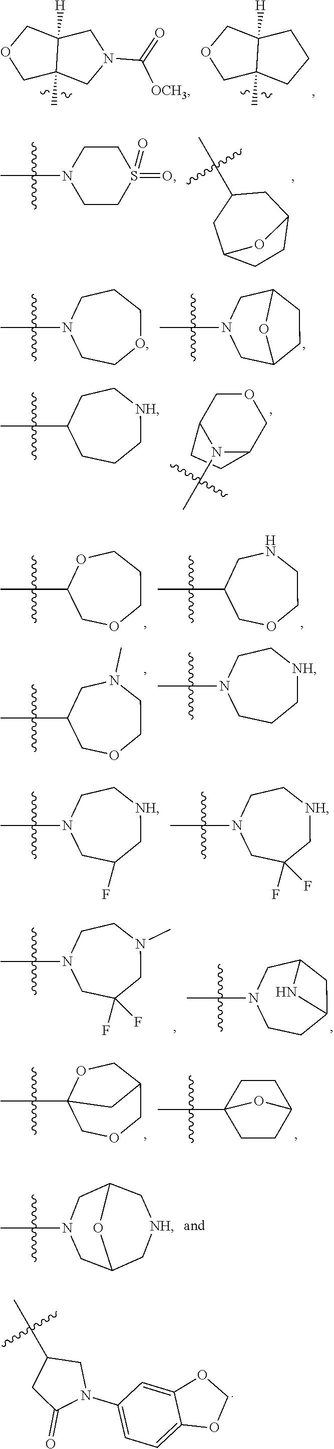

In some embodiments, R² is selected from among

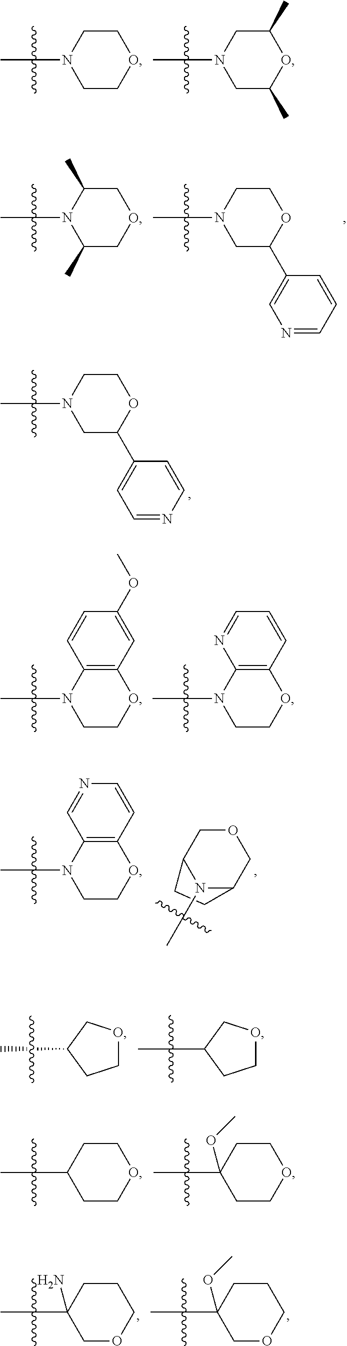

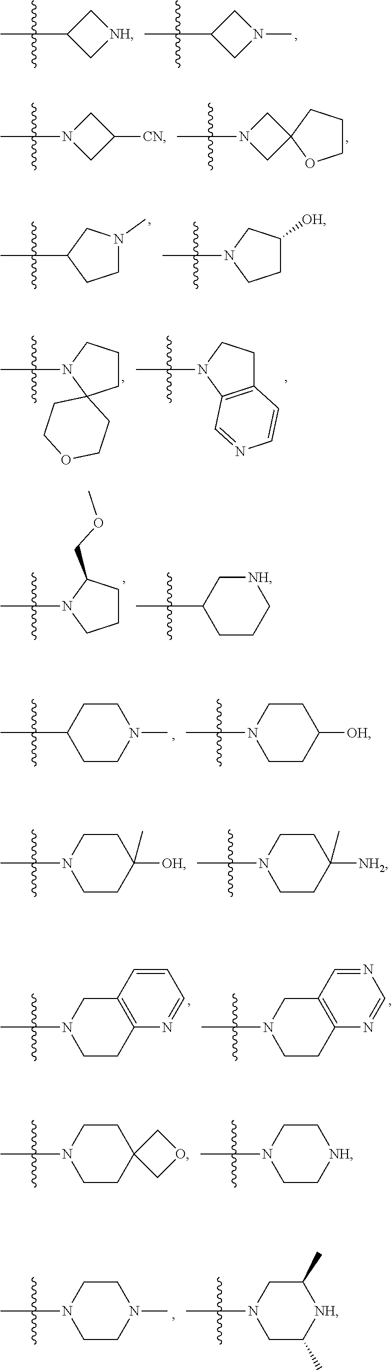

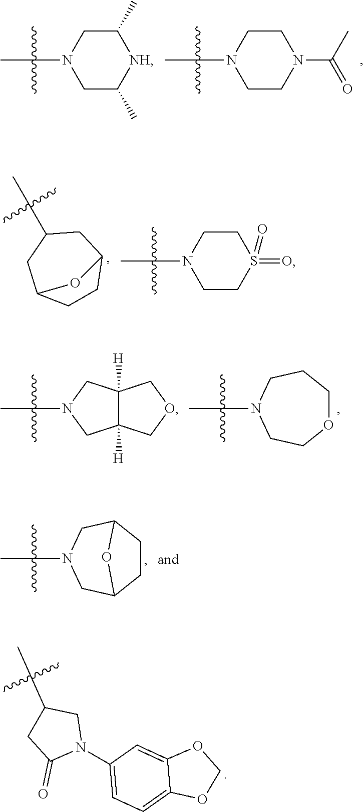

In certain embodiments, R² is 5-10 membered heteroaryl, wherein the 5-10 membered heteroaryl is optionally substituted with C₁₋₆ alkyl optionally substituted with halogen or —OR^{2a}, —OH, —OR a, oxo, halogen, —C(O)R^{2a}, —C(OO)R^{2a}, —C(O)NR^{2b}R^{2c}, —CN, —NR^{2b}R^{2c}, 3-6 membered cycloalkyl, 3-7 membered heterocyclyl, 6-10 membered aryl, or 5-10 membered heteroaryl; wherein R^{2a} is H, C₁₋₆ alkyl, C₁₋₆ haloalkyl, 3-7 membered heterocyclyl, or —(CH₂)ᵣOCH₃, wherein r is 1, 2, or 3; wherein R^{2b} is H or C₁₋₆ alkyl; and wherein R^{2c} is H or C₁₋₆ alkyl.

In certain embodiments, R² is 5-10 membered heteroaryl, wherein the 5-10 membered heteroaryl is optionally substituted with C₁₋₆ alkyl, —OH, halogen, —C(O)R^{2a}, or —C(O)NR^{2b}R^{2c}; wherein R^{2a} is C₁₋₆ alkyl or —(CH₂)ᵣOCH₃, wherein r is 1, 2, or 3; wherein R^{2b} is H or C₁₋₆ alkyl; and wherein R^{2c} is H or C₁₋₆ alkyl.

In some embodiments, R² is selected from among

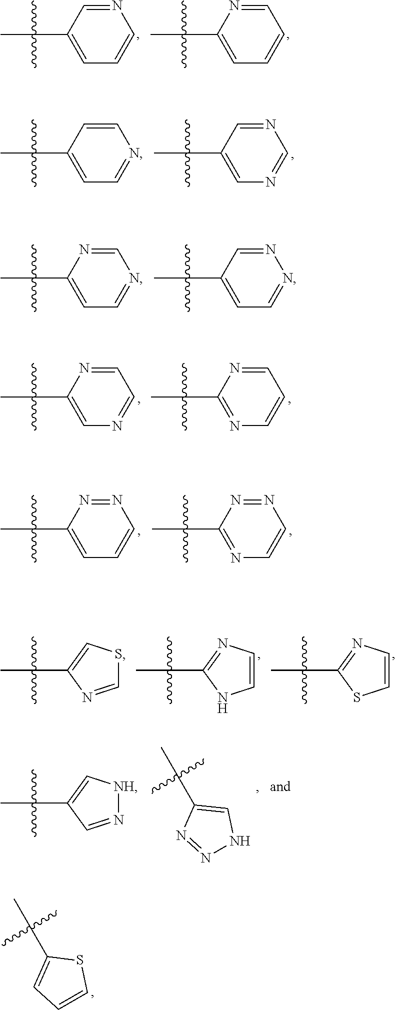

each of which may be substituted at, e.g., a carbon atom, nitrogen atom, or sulfur atom. The 5-10 membered heteroaryl may be monocyclic or polycyclic, including fused rings with aryl, heteroaryl, cycloalkyl, or heterocyclyl rings.

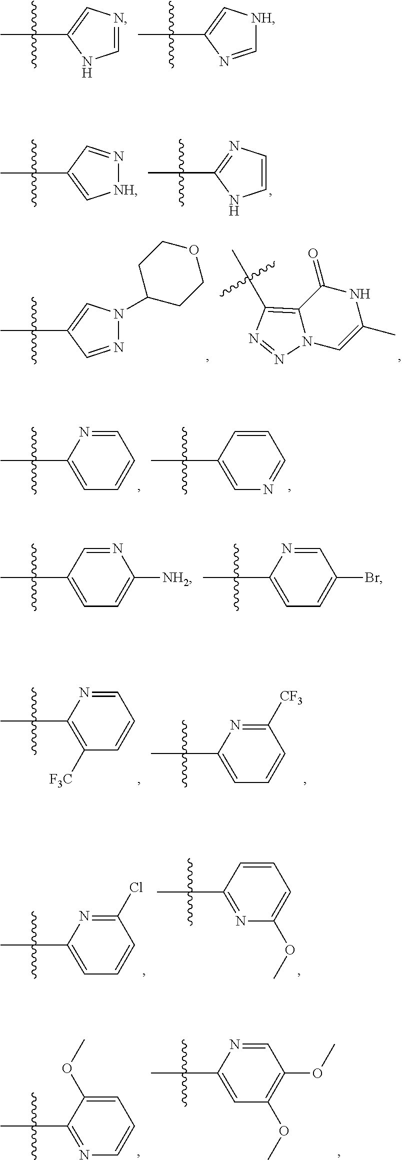

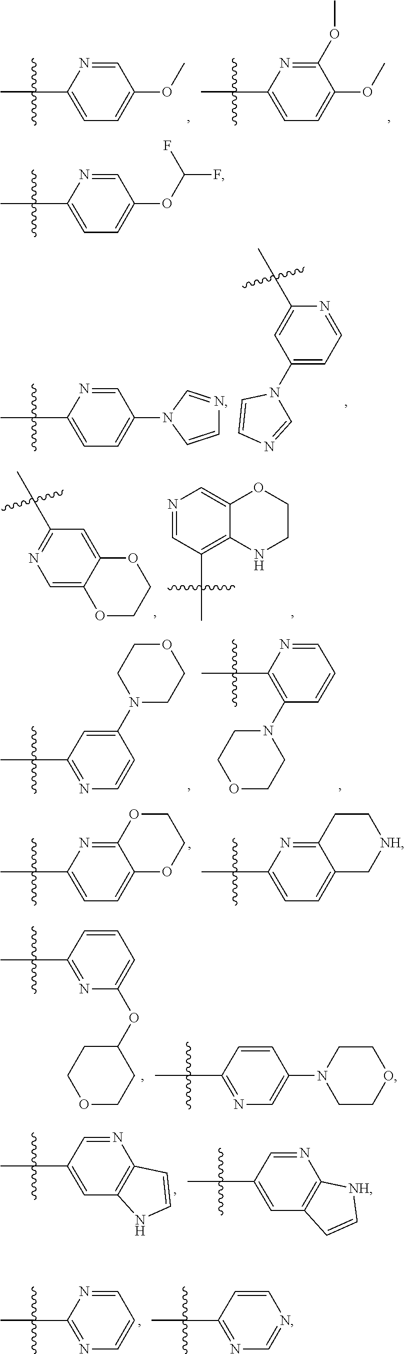

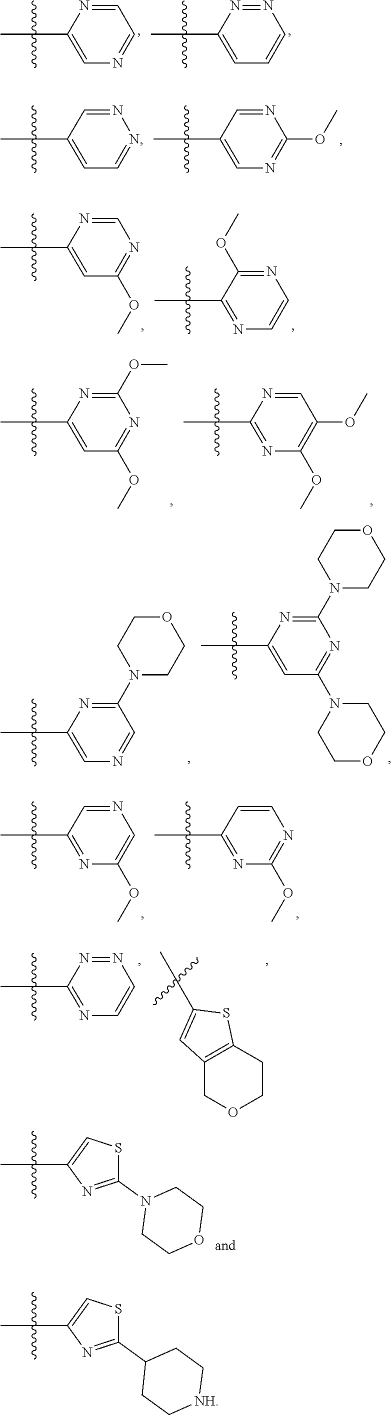

In some embodiments, R² is selected from among

In certain embodiments, R² is 6-10 membered aryl, wherein the 6-10 membered aryl is optionally substituted with C₁₋₆ alkyl optionally substituted with halogen or —OR^{2a}, —OH, —OR^{2a}, oxo, halogen, —C(O)R^{2a}, —C(OO)R^{2a}, —C(O)NR^{2b}R^{2c}, —CN, —NR^{2b}R^{2c}, 3-6 membered cycloalkyl, 3-7 membered heterocyclyl, 6-10 membered aryl, or 5-10 membered heteroaryl; wherein R^{2a} is H, C₁₋₆ alkyl, C₁₋₆haloalkyl, 3-7 membered heterocyclyl, or —(CH₂)ᵣOCH₃, wherein r is 1, 2, or 3; wherein R^{2b} is H or C₁₋₆ alkyl; and wherein R^{2e} is H or C₁₋₆ alkyl.

In certain embodiments, R² is 6-10 membered aryl, wherein the 6-10 membered aryl is optionally substituted with C₁₋₆ alkyl, —OH, halogen, —C(O)R^{2a}, or —C(O)NR^{2b}R^{2c}; wherein R^{2a} is C₁₋₆ alkyl or —(CH₂)ᵣOCH₃, wherein r is 1, 2, or 3; wherein R^{2b} is H or C₁₋₆ alkyl; and wherein R^{2c} is H or C₁₋₆ alkyl.

In some embodiments, R² is a phenyl ring, which is optionally substituted. The phenyl ring may comprise a fused ring, including to heteroaryl, heterocyclyl, and cycloalkyl rings. In some embodiments, R² is selected from among

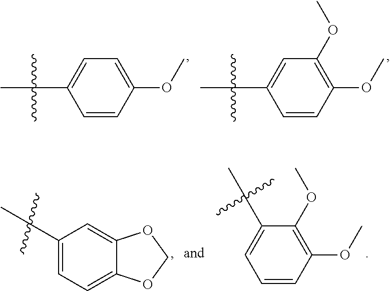

In certain embodiments, R² is 3-14 membered cycloalkyl, wherein the 3-14 membered cycloalkyl is optionally substituted with C₁₋₆ alkyl optionally substituted with halogen or —OR^{2a}, —OH, —OR^{2a}, oxo, halogen, —C(O)R^{2a}, —C(OO)R^{2a}, —C(O)NR^{2b}R^{2c}, —CN, —NR^{2b}R^{2c}, 3-6 membered cycloalkyl, 3-7 membered heterocyclyl, 6-10 membered aryl, or 5-10 membered heteroaryl; wherein R^{2a} is H, C₁₋₆ alkyl, C₁₋₆ haloalkyl, 3-7 membered heterocyclyl, or —(CH₂)ᵣOCH₃, wherein r is 1, 2, or 3; wherein R^{2b} is H or C₁₋₆ alkyl; and wherein R^{2c} is H or C₁₋₆ alkyl.

In certain embodiments, R² is 3-14 membered cycloalkyl, wherein the 3-14 membered cycloalkyl is optionally substituted with C₁₋₆ alkyl, —OH, halogen, —C(O)R^{2a}, or —C(O)NR^{2b}R^{2c}; wherein R^{2a} is C₁₋₆ alkyl or —(CH₂)ᵣOCH₃, wherein r is 1, 2, or 3; wherein R^{2b} is H or C₁₋₆ alkyl; and wherein R^{2c} is H or C₁₋₆ alkyl.

In some embodiments, R² is selected from among cyclopropyl, cyclobutyl, cyclopentyl, and cyclohexyl, each of which is optionally substituted. The cycloalkyl may comprise a fused ring, including to aryl (e.g., 6-10 membered aryl), heteroaryl (e.g., 5-10 membered heteroaryl), heterocyclyl (e.g., 3-14 membered heterocyclyl), and cycloalkyl (e.g., 3-8 membered cycloalkyl) rings.

In some embodiments, R² is selected from among

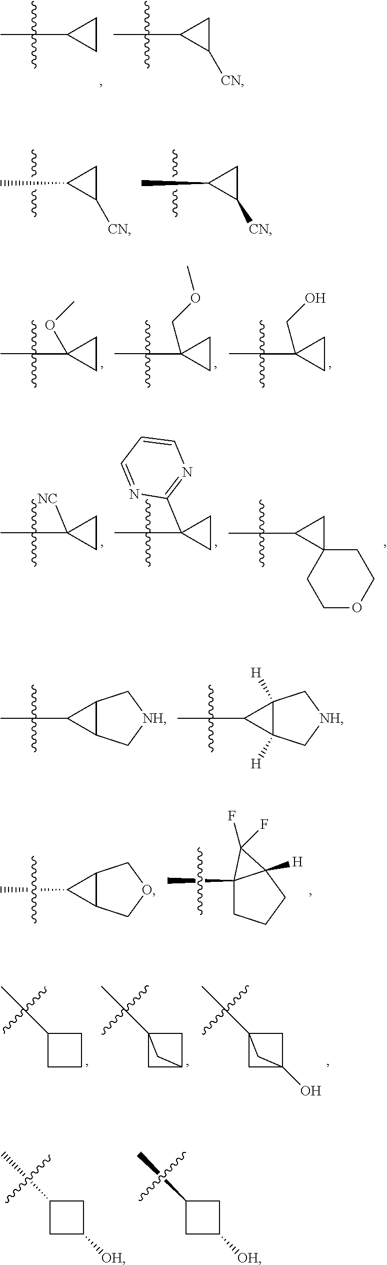

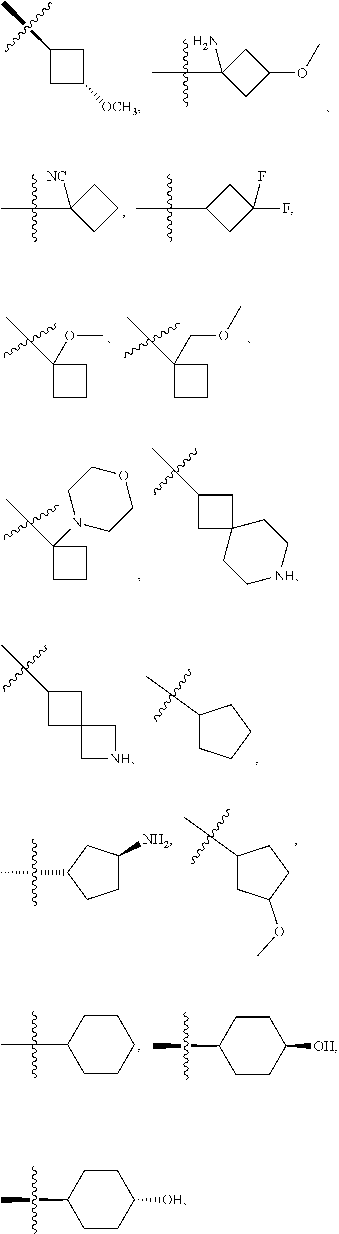

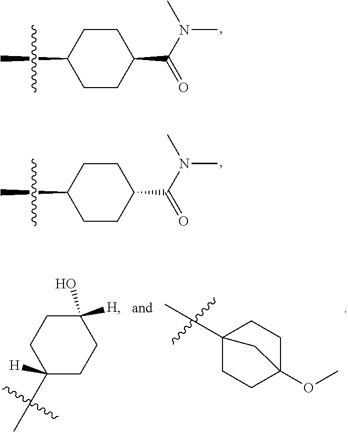

In some embodiments, R² is selected from among

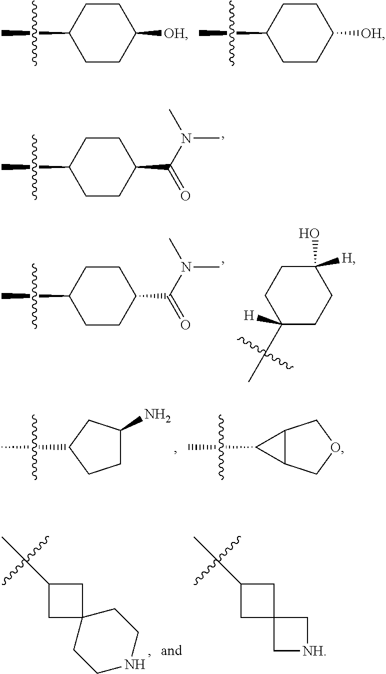

In certain embodiments, R² is 3-14 membered cycloalkenyl, wherein the 3-14 membered cycloalkenyl is optionally substituted with C₁₋₆ alkyl optionally substituted with halogen or —OR^{2a}, —OH, —OR^{2a} oxo, halogen, —C(O)R^{2a}, —C(OO)R^{2a}, —C(O)NR^{2b}R^{2c}, —CN, —NR^{2b}R^{2c}, 3-6 membered cycloalkyl, 3-7 membered heterocyclyl, 6-10 membered aryl, or 5-10 membered heteroaryl; wherein R^{2a} is H, C₁₋₆ alkyl, C₁₋₆ haloalkyl, 3-7 membered heterocyclyl, or —(CH₂)ᵣOCH₃, wherein r is 1, 2, or 3; wherein R^{2b} is H or C₁₋₆ alkyl; and wherein R^{2c} is H or C₁₋₆ alkyl.

In certain embodiments, R² is 3-14 membered cycloalkenyl, wherein the 3-14 membered cycloalkenyl is optionally substituted with C₁₋₆ alkyl, —OH, halogen, —C(O)R^{2a} or —C(O)NR^{2b}R^{2c}; wherein R^{2a} is C₁₋₆ alkyl or —(CH₂)ᵣOCH₃, wherein r is 1, 2, or 3; wherein R^{2b} is H or C₁₋₆ alkyl; and wherein R^{2c} is H or C₁₋₆ alkyl.

In certain embodiments, L² is a bond and R² is H.

In some embodiments, R³ and R⁴ are independently H or C₁₋₆ alkyl optionally substituted with halo or —OH; wherein at least one of R³ and R⁴ is H or wherein R³ and R⁴ together with the atom to which they are attached combine to form a 3-6 membered cycloalkyl.

In some embodiments, R³ and R⁴ are independently selected from the group consisting of H and C₁₋₆ alkyl; wherein at least one of R³ and R⁴ is not H; or R³ and R⁴ together with the atom to which they are attached combine to form a 3-6 membered cycloalkyl.

In certain embodiments, R³ is H. In certain embodiments, R³ is C₁₋₆ alkyl, such as C₁ alkyl, C₂ alkyl, C₃ alkyl, C₄ alkyl, C₅ alkyl, or C₆ alkyl.

In certain embodiments, R⁴ is H. In certain embodiments, R⁴ is C₁₋₆ alkyl, such as C₁ alkyl, C₂ alkyl, C₃ alkyl, C₄ alkyl, C₅ alkyl, or C₆ alkyl.

In certain embodiments, R³ is H and R⁴ is C₁₋₆ alkyl, such as Cl alkyl, C₂ alkyl, C₃ alkyl, C₄ alkyl, C₅ alkyl, or C₆ alkyl.

In certain embodiments, R³ and R⁴ together with the atom to which they are attached combine to form a 3-6 membered cycloalkyl, such as 3, 4, 5 or 6-membered cycloalkyl.

In some embodiments, R⁵, R⁶, R⁷, R⁸, and R⁹ are independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OH, halogen, —NO₂, —CN, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, halogen, —NO₂, oxo, —CN, —R¹⁰, —OR¹⁰, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, 3-8 membered cycloalkyl, 3-14 membered heterocyclyl, 6-10 membered aryl, or 5-10 membered heteroaryl

In some embodiments, R⁵, R⁶, R⁷, R⁸, and R⁹ are independently selected from the group consisting of H, D, C₁₋₆ alkyl, C₂₋₆ alkenyl, 4-8 membered cycloalkenyl, C₂₋₆ alkynyl, 3-8 membered cycloalkyl, —OH, halogen, —NO₂, —CN, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, and 3-8 membered cycloalkyl are independently optionally substituted with —OH, halogen, —NO₂, oxo, —CN, —R¹⁰, —OR¹⁰, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, 3-14 membered heterocyclyl, 6-10 membered aryl, or 5-10 membered heteroaryl.

In some embodiments, any two adjacent R⁵, R⁶, R⁷, R⁸, and R⁹ form a 3-14 membered fused ring, or a 3-10 membered fused ring, or a 3-6 membered fused ring. Stated another way, in some embodiments, A is a fused bicyclic group containing 18 ring atoms or fewer, 14 ring atoms or fewer, or 10 ring atoms of fewer. The fused ring may be a 3-8 membered cycloalkyl, a 4-8 membered cycloalkenyl, a 3-14 membered heterocyclyl, or a 3-8 membered heteroaryl. In some embodiments, the bicyclic ring is optionally substituted with one to three substituents selected from among C₁₋₆ alkyl optionally substituted with —OH, —OH, halogen, —NO₂, oxo, —CN, —R¹⁰, —OR¹⁰, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, 3-14 membered heterocyclyl, 6-10 membered aryl, or 5-10 membered heteroaryl.

In some embodiments, R¹⁰, R¹¹, and R¹² are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR¹³, —SR¹³, halogen, —NR¹³R¹⁴, —NO₂, and —CN.

In some embodiments, R¹³ and R¹⁴ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₆ alkynyl, 3-8 membered cycloalkyl, and 3-14 membered heterocyclyl are independently optionally substituted with —OH, —SH, —NH₂, —NO₂, or —CN.

In certain embodiments, one to three of R₅, R₆, R₇, R₈, and R₉ is C₁₋₆ alkyl optionally substituted with halogen. In certain embodiments, one to three of R⁵, R⁶, R⁷, R⁸, and R⁹ is CF₃. In certain embodiments, one to three of R⁵, R⁶, R⁷, R⁸, and R⁹ is CHF₂.

In certain embodiments, one to three of R₅, R₆, R₇, R₈, and R₉ is C₁₋₆ alkyl optionally substituted with halogen or —OH. In certain embodiments, one to three of R₅, R₆, R₇, R₈, and R₉ is C₁₋₆ alkyl optionally substituted with fluorine and —OH.

In certain embodiments, one to three of R₅, R₆, R₇, R₈, and R₉ is halogen, and one to three of R₅, R₆, R₇, R₈, and R₉ is C₁₋₆ alkyl optionally substituted with halogen. In certain embodiments, one to three of R₅, R₆, R₇, R₈, and R₉ is fluorine, and one to three of R₅, R₆, R₇, R₈, and R₉ is C₁₋₆ alkyl optionally substituted with fluorine.

In certain embodiments, one to three of R⁵, R⁶, R⁷, R⁸, and R⁹ is —NH₂.

In certain embodiments, one of R₅, R₆, R₇, R₈, and R₉ is —NH₂; and one of R₅, R₆, R₇, R₈, and R₉ is C₁₋₆ alkyl optionally substituted with halogen. In certain embodiments, one of R⁵, R⁶, R⁷, R⁸, and R⁹ is —NH₂; and one of R⁵, R⁶, R⁷, R⁸, and R⁹ is CF₃.

In some embodiments, A is selected from among:

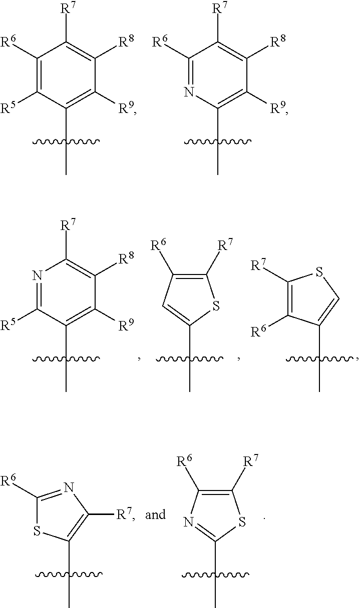

In some embodiments, A is selected from among:

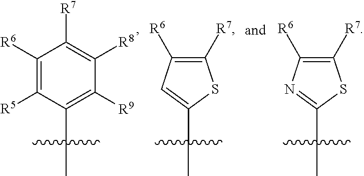

In some embodiments, A is selected from among:

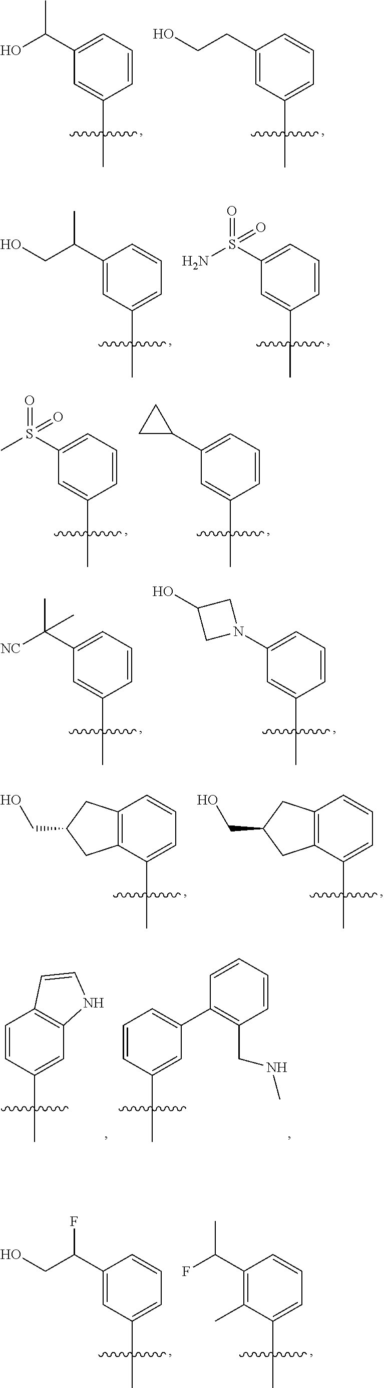

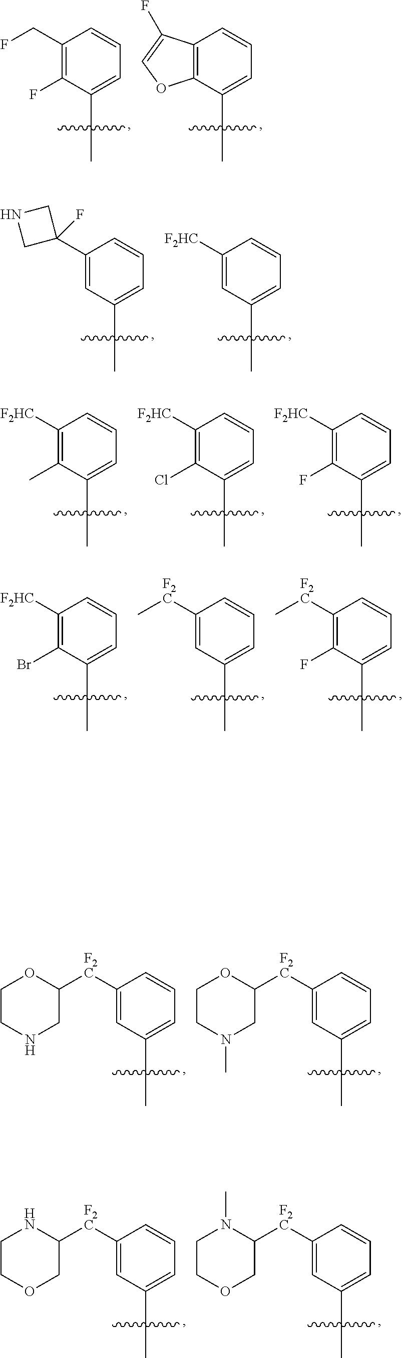

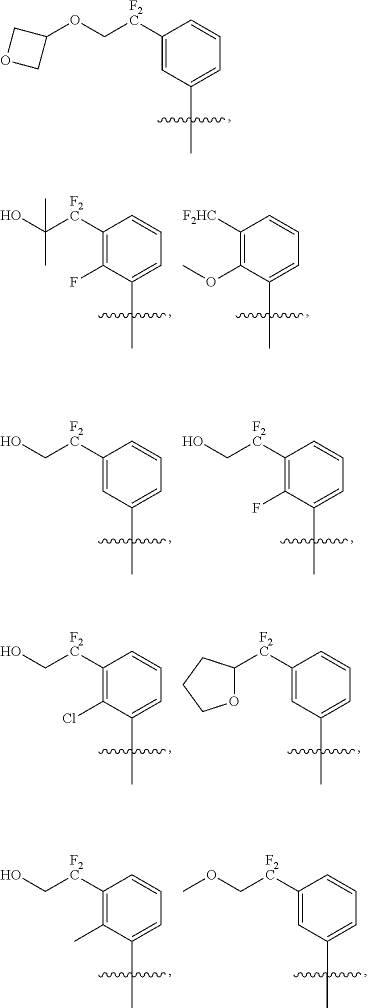

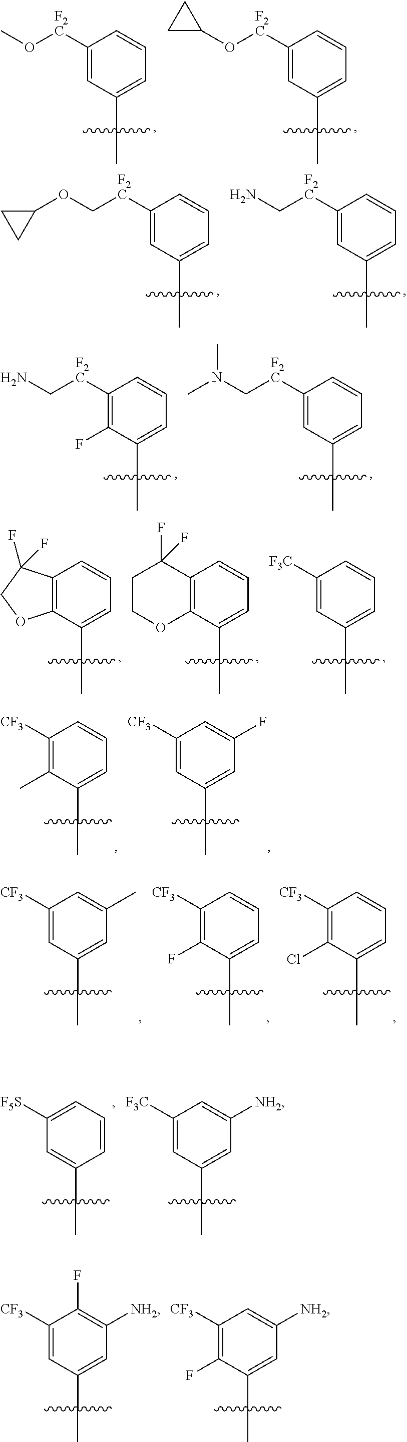

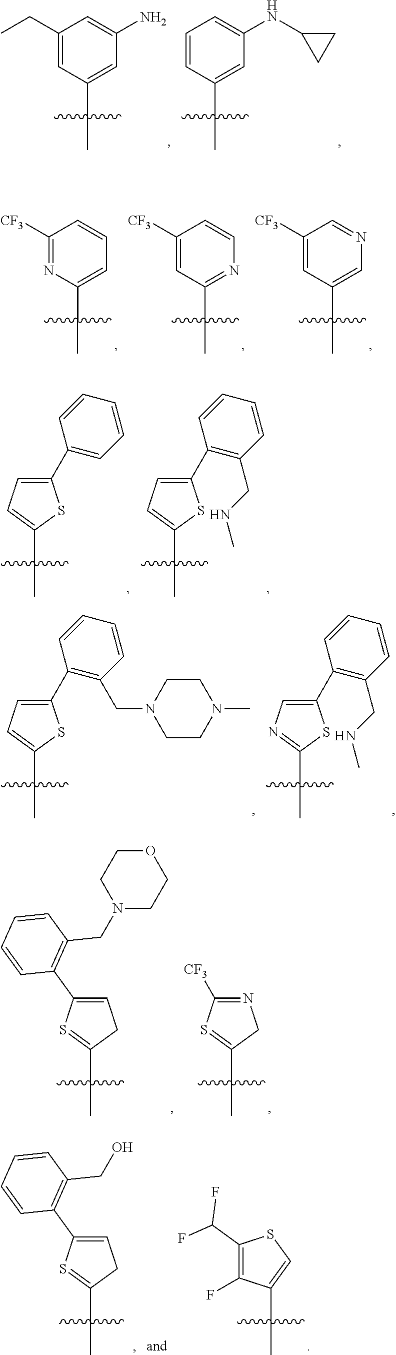

In some embodiments, A is selected from among:

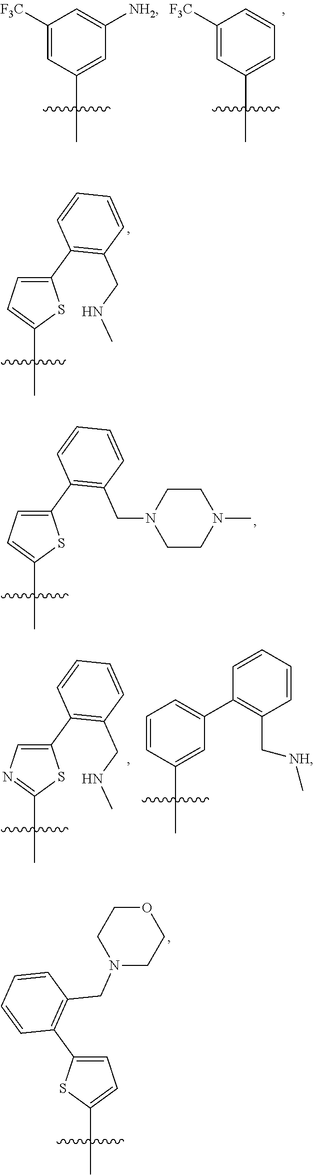

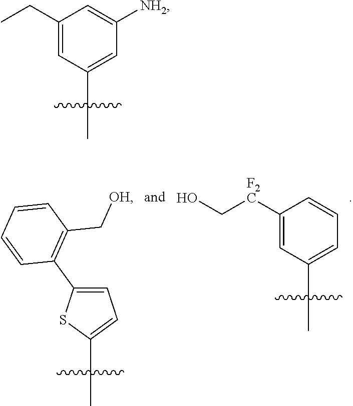

In some embodiments, the compound of formula (I-a) or (V-a), or a pharmaceutically acceptable salt, solvate, stereoisomer, or tautomer thereof, has one, two, three or more of the following features:
a)

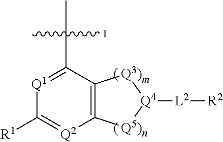

is selected from the group consisting of

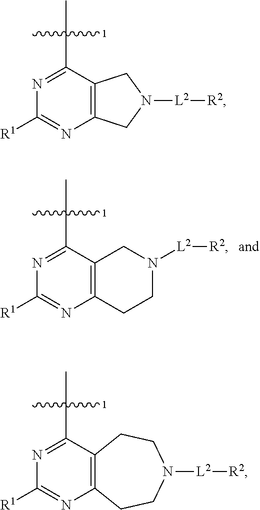

wherein 1 is a point of connection of the A ring to the nitrogen atom;
b) R³ is H and R⁴ is C₁₋₆ alkyl;
c) L² is a bond or —C(O)—;
d) R² is optionally substituted (e.g., 3-8 membered cycloalkyl), optionally substituted cycloalkenyl (e.g., 4-8 membered cycloalkenyl), or optionally substituted heterocyclyl (e.g., 3-14 membered heterocyclyl).

In some embodiments, the compound of formula (V-a), or a pharmaceutically acceptable salt, solvate, stereoisomer, or tautomer thereof, has one, two, three or more of the following features:
a)

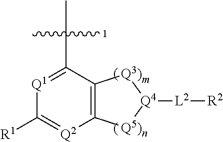

is selected from the group consisting of

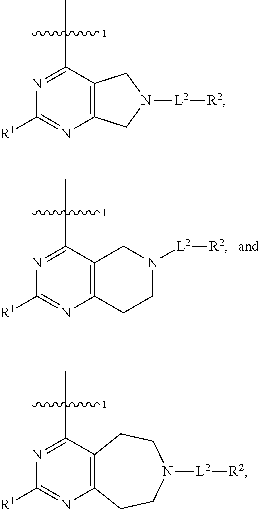

wherein 1 is a point of connection of the A ring to the nitrogen atom;
b) one to three of R⁵, R⁶, R⁷, R⁸, and R⁹ is C₁₋₆ alkyl, wherein the alkyl is optionally substituted with one or more halogen atoms;
c) R³ is H and R⁴ is C₁₋₆ alkyl;
d) L² is a bond or —C(O)—;
e) R² is optionally substituted cycloalkyl (e.g., 3-8 membered cycloalkyl), optionally substituted cycloalkenyl (e.g., 4-8 membered cycloalkenyl), or optionally substituted heterocyclyl (e.g., 3-14 membered heterocyclyl).

The present disclosure provides compound of formula (I-a), or a pharmaceutically acceptable salt, solvate, isomer, prodrug, or tautomer thereof, which is

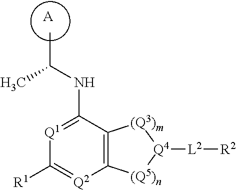

wherein A, L², Q¹, Q², Q³, Q⁴, Q⁵, R¹, R², m and n are as defined above.

The present disclosure provides compound of formula (V-a), or a pharmaceutically acceptable salt, solvate, isomer, prodrug, or tautomer thereof, which is

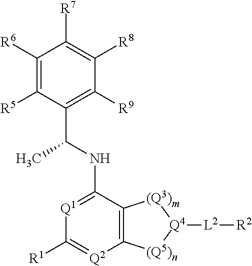

wherein L², Q¹, Q², Q³, Q⁴, Q⁵, R¹, R², R⁴, R⁵, R⁶, R⁷, R⁸, R⁹, m and n are as defined above.

The present disclosure provides a compound, and pharmaceutically acceptable salts, solvates, stereoisomers, and tautomers thereof, selected from the group consisting of compounds of Table A:

TABLE A

| Example # | Structure |
|---|---|
| Example 1. | 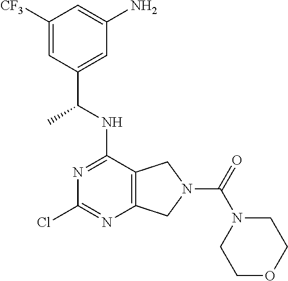 [SMILES: C[C@@H](Nc1nc(Cl)nc2c1CN(C(=O)N1CCOCC1)C2)c1cc(N)cc(C(F)(F)F)c1] |
| Example 2. | 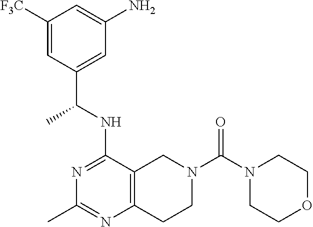 [SMILES: Cc1nc2c(c(N[C@H](C)c3cc(N)cc(C(F)(F)F)c3)n1)CN(C(=O)N1CCOCC1)CC2] |
| Example 3. | 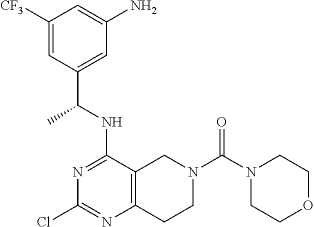 [SMILES: C[C@@H](Nc1nc(Cl)nc2c1CN(C(=O)N1CCOCC1)CC2)c1cc(N)cc(C(F)(F)F)c1] |
| Example 4. | 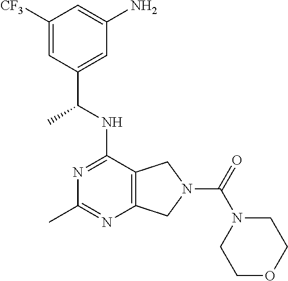 [SMILES: Cc1nc2c(c(N[C@H](C)c3cc(N)cc(C(F)(F)F)c3)n1)CN(C(=O)N1CCOCC1)C2] |
| Example 5. | 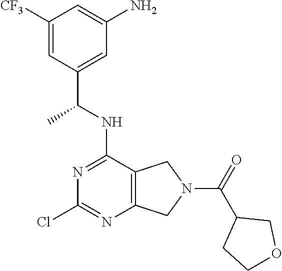 [SMILES: C[C@@H](Nc1nc(Cl)nc2c1CN(C(=O)C1CCOC1)C2)c1cc(N)cc(C(F)(F)F)c1] |
| Example 6. | 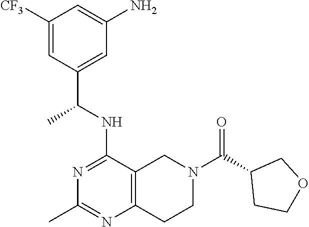 [SMILES: Cc1nc2c(c(N[C@H](C)c3cc(N)cc(C(F)(F)F)c3)n1)CN(C(=O)[C@H]1CCOC1)CC2] |
| Example 7. | 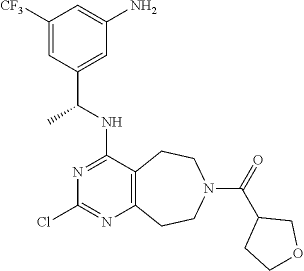 [SMILES: C[C@@H](Nc1nc(Cl)nc2c1CCN(C(=O)C1CCOC1)CC2)c1cc(N)cc(C(F)(F)F)c1] |
| Example 8. | 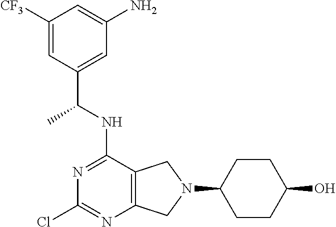 [SMILES: C[C@@H](Nc1nc(Cl)nc2c1CN([C@H]1CC[C@@H](O)CC1)C2)c1cc(N)cc(C(F)(F)F)c1] |
| Example 9. | 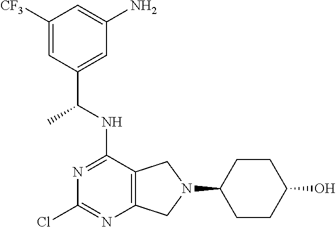 [SMILES: C[C@@H](Nc1nc(Cl)nc2c1CN([C@H]1CC[C@H](O)CC1)C2)c1cc(N)cc(C(F)(F)F)c1] |
| Example 10. | 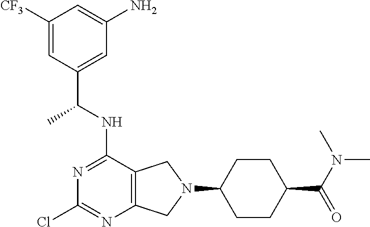 [SMILES: C[C@@H](Nc1nc(Cl)nc2c1CN([C@H]1CC[C@@H](C(=O)N(C)C)CC1)C2)c1cc(N)cc(C(F)(F)F)c1] |
| Example 11. | 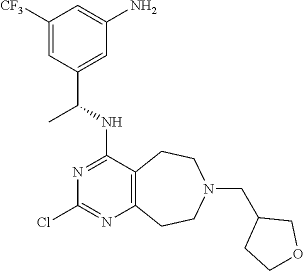 [SMILES: C[C@@H](Nc1nc(Cl)nc2c1CCN(CC1CCOC1)CC2)c1cc(N)cc(C(F)(F)F)c1] |
| Example 12. | 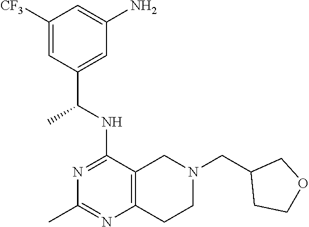 [SMILES: Cc1nc2c(c(N[C@H](C)c3cc(N)cc(C(F)(F)F)c3)n1)CN(CC1CCOC1)CC2] |
| Example 13. | 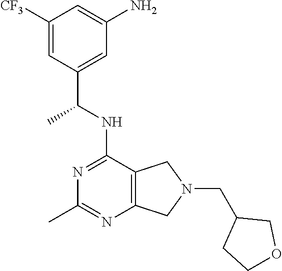 [SMILES: Cc1nc2c(c(N[C@H](C)c3cc(N)cc(C(F)(F)F)c3)n1)CN(CC1CCOC1)C2] |
| Example 14. | 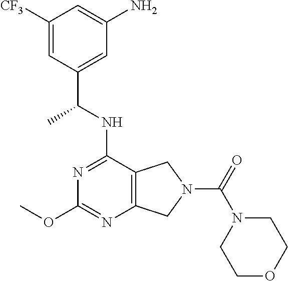 [SMILES: COc1nc2c(c(N[C@H](C)c3cc(N)cc(C(F)(F)F)c3)n1)CN(C(=O)N1CCOCC1)C2] |
| Example 15. | 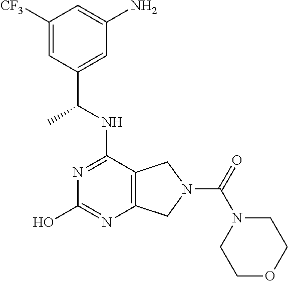 [SMILES: C[C@@H](Nc1nc(O)nc2c1CN(C(=O)N1CCOCC1)C2)c1cc(N)cc(C(F)(F)F)c1] |
| Example 16. | 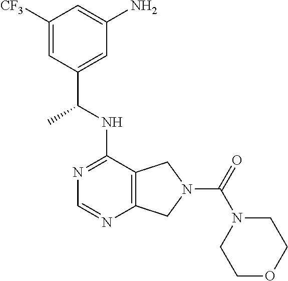 [SMILES: C[C@@H](Nc1ncnc2c1CN(C(=O)N1CCOCC1)C2)c1cc(N)cc(C(F)(F)F)c1] |
| Example 17. | 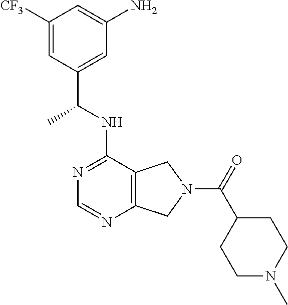 [SMILES: C[C@@H](Nc1ncnc2c1CN(C(=O)C1CCN(C)CC1)C2)c1cc(N)cc(C(F)(F)F)c1] |
| Example 18. | 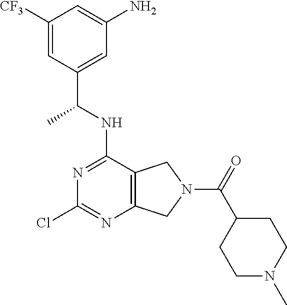 [SMILES: C[C@@H](Nc1nc(Cl)nc2c1CN(C(=O)C1CCN(C)CC1)C2)c1cc(N)cc(C(F)(F)F)c1] |
| Example 19. | 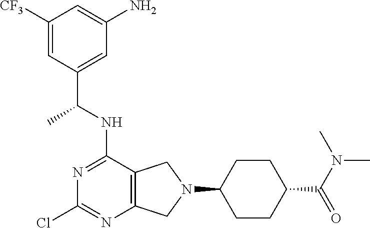 [SMILES: C[C@@H](Nc1nc(Cl)nc2c1CN([C@H]1CC[C@H](C(=O)N(C)C)CC1)C2)c1cc(N)cc(C(F)(F)F)c1] |
| Example 20. | 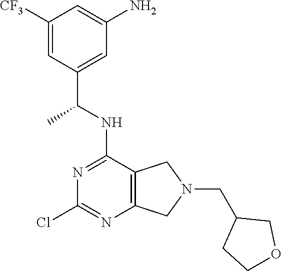 [SMILES: C[C@@H](Nc1nc(Cl)nc2c1CN(CC1CCOC1)C2)c1cc(N)cc(C(F)(F)F)c1] |
| Example 21. | 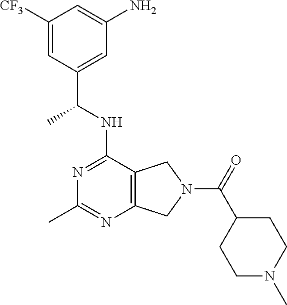 [SMILES: Cc1nc2c(c(N[C@H](C)c3cc(N)cc(C(F)(F)F)c3)n1)CN(C(=O)C1CCN(C)CC1)C2] |
| Example 22. | 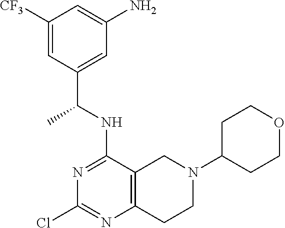 [SMILES: C[C@@H](Nc1nc(Cl)nc2c1CN(C1CCOCC1)CC2)c1cc(N)cc(C(F)(F)F)c1] |
| Example 23. | 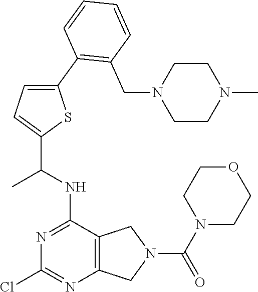 [SMILES: CC(Nc1nc(Cl)nc2c1CN(C(=O)N1CCOCC1)C2)c1ccc(-c2ccccc2CN2CCN(C)CC2)s1] |
| Example 24. | 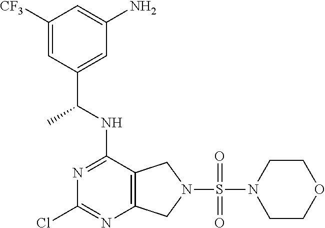 [SMILES: C[C@@H](Nc1nc(Cl)nc2c1CN(S(=O)(=O)N1CCOCC1)C2)c1cc(N)cc(C(F)(F)F)c1] |
| Example 25. | 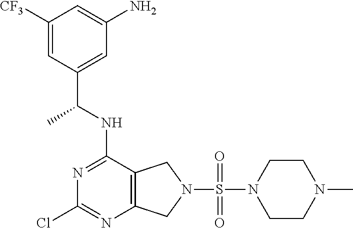 [SMILES: C[C@@H](Nc1nc(Cl)nc2c1CN(S(=O)(=O)N1CCN(C)CC1)C2)c1cc(N)cc(C(F)(F)F)c1] |
| Example 26. | 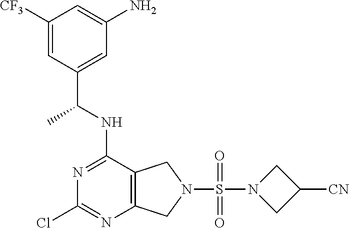 [SMILES: C[C@@H](Nc1nc(Cl)nc2c1CN(S(=O)(=O)N1CC(C#N)C1)C2)c1cc(N)cc(C(F)(F)F)c1] |
| Example 27. | 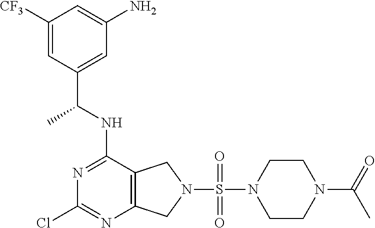 [SMILES: CC(=O)N1CCN(S(=O)(=O)N2Cc3nc(Cl)nc(N[C@H](C)c4cc(N)cc(C(F)(F)F)c4)c3C2)CC1] |
| Example 28. | 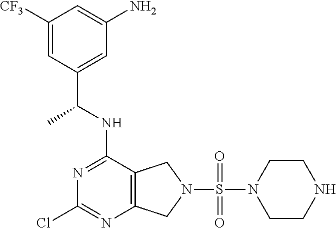 [SMILES: C[C@@H](Nc1nc(Cl)nc2c1CN(S(=O)(=O)N1CCNCC1)C2)c1cc(N)cc(C(F)(F)F)c1] |
| Example 29. | 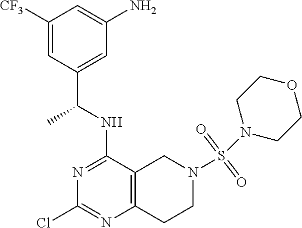 [SMILES: C[C@@H](Nc1nc(Cl)nc2c1CN(S(=O)(=O)N1CCOCC1)CC2)c1cc(N)cc(C(F)(F)F)c1] |
| Example 30. | 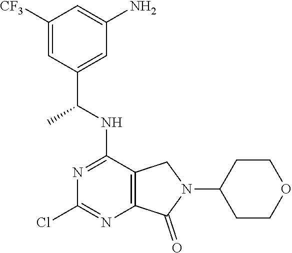 [SMILES: C[C@@H](Nc1nc(Cl)nc2c1CN(C1CCOCC1)C2=O)c1cc(N)cc(C(F)(F)F)c1] |
| Example 31. | 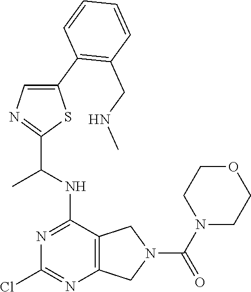 [SMILES: CNCc1ccccc1-c1cnc(C(C)Nc2nc(Cl)nc3c2CN(C(=O)N2CCOCC2)C3)s1] |
| Example 32. | 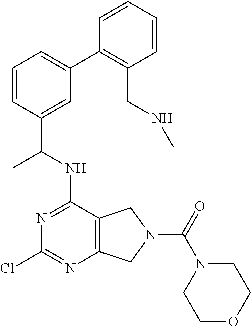 [SMILES: CNCc1ccccc1-c1cccc(C(C)Nc2nc(Cl)nc3c2CN(C(=O)N2CCOCC2)C3)c1] |
| Example 33. | 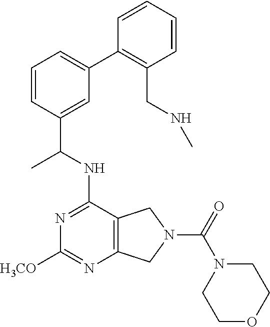 [SMILES: CNCc1ccccc1-c1cccc(C(C)Nc2nc(OC)nc3c2CN(C(=O)N2CCOCC2)C3)c1] |
| Example 34. | 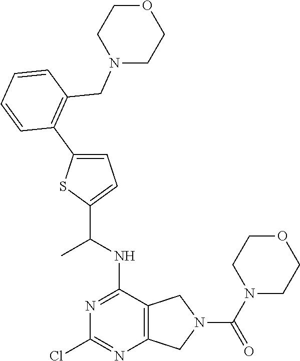 [SMILES: CC(Nc1nc(Cl)nc2c1CN(C(=O)N1CCOCC1)C2)c1ccc(-c2ccccc2CN2CCOCC2)s1] |
| Example 35. | 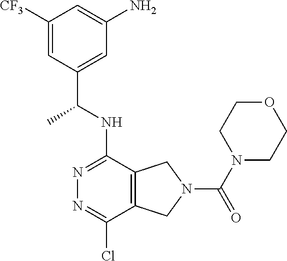 [SMILES: C[C@@H](Nc1nnc(Cl)c2c1CN(C(=O)N1CCOCC1)C2)c1cc(N)cc(C(F)(F)F)c1] |
| Example 36. | 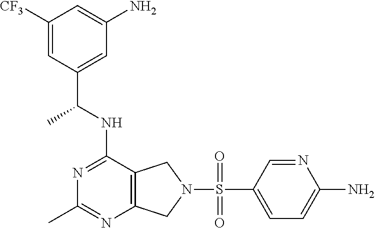 [SMILES: Cc1nc2c(c(N[C@H](C)c3cc(N)cc(C(F)(F)F)c3)n1)CN(S(=O)(=O)c1ccc(N)nc1)C2] |
| Example 37. | 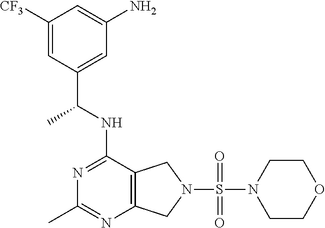 [SMILES: Cc1nc2c(c(N[C@H](C)c3cc(N)cc(C(F)(F)F)c3)n1)CN(S(=O)(=O)N1CCOCC1)C2] |
| Example 38. | 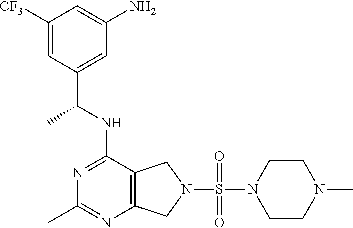 [SMILES: Cc1nc2c(c(N[C@H](C)c3cc(N)cc(C(F)(F)F)c3)n1)CN(S(=O)(=O)N1CCN(C)CC1)C2] |
| Example 39. | 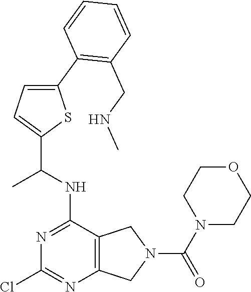 [SMILES: CNCc1ccccc1-c1ccc(C(C)Nc2nc(Cl)nc3c2CN(C(=O)N2CCOCC2)C3)s1] |
| Example 40. | 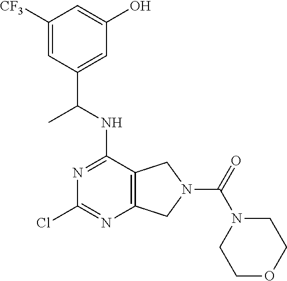 [SMILES: CC(Nc1nc(Cl)nc2c1CN(C(=O)N1CCOCC1)C2)c1cc(O)cc(C(F)(F)F)c1] |
| Example 41. | 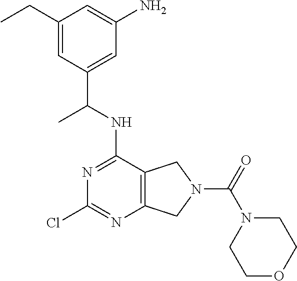 [SMILES: CCc1cc(N)cc(C(C)Nc2nc(Cl)nc3c2CN(C(=O)N2CCOCC2)C3)c1] |
| Example 42. | 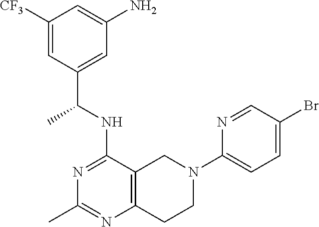 [SMILES: Cc1nc2c(c(N[C@H](C)c3cc(N)cc(C(F)(F)F)c3)n1)CN(c1ccc(Br)cn1)CC2] |
| Example 43. | 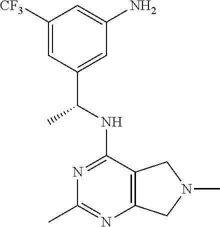 [SMILES: Cc1nc2c(c(N[C@H](C)c3cc(N)cc(C(F)(F)F)c3)n1)CN(C)C2] |
| Example 44. | 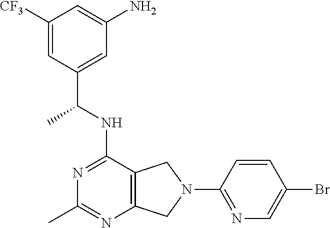 [SMILES: Cc1nc2c(c(N[C@H](C)c3cc(N)cc(C(F)(F)F)c3)n1)CN(c1ccc(Br)cn1)C2] |
| Example 45. | 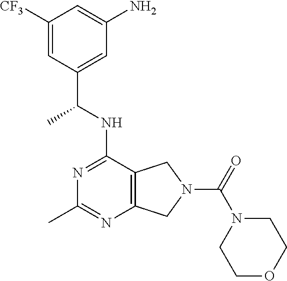 [SMILES: Cc1nc2c(c(N[C@H](C)c3cc(N)cc(C(F)(F)F)c3)n1)CN(C(=O)N1CCOCC1)C2] |
| Example 46. | 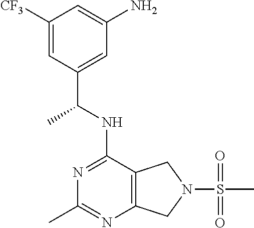 [SMILES: Cc1nc2c(c(N[C@H](C)c3cc(N)cc(C(F)(F)F)c3)n1)CN(S(C)(=O)=O)C2] |
| Example 47. | 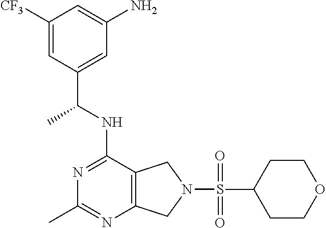 [SMILES: Cc1nc2c(c(N[C@H](C)c3cc(N)cc(C(F)(F)F)c3)n1)CN(S(=O)(=O)C1CCOCC1)C2] |
| Example 48., 166. | 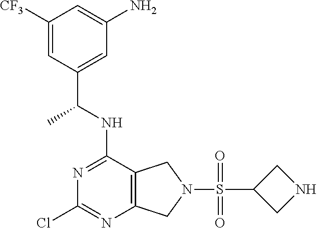 [SMILES: C[C@@H](Nc1nc(Cl)nc2c1CN(S(=O)(=O)C1CNC1)C2)c1cc(N)cc(C(F)(F)F)c1] |
| Example 49. | 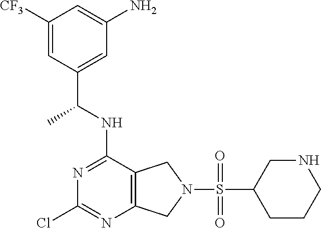 [SMILES: C[C@@H](Nc1nc(Cl)nc2c1CN(S(=O)(=O)C1CCCNC1)C2)c1cc(N)cc(C(F)(F)F)c1] |
| Example 50. | 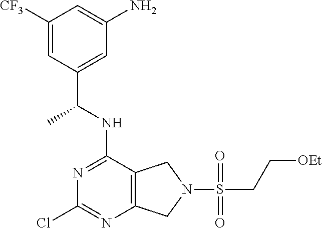 [SMILES: CCOCCS(=O)(=O)N1Cc2nc(Cl)nc(N[C@H](C)c3cc(N)cc(C(F)(F)F)c3)c2C1] |
| Example 51. | 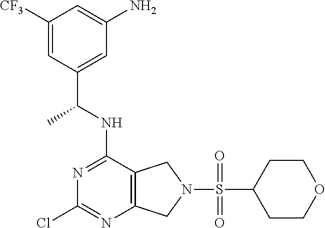 [SMILES: C[C@@H](Nc1nc(Cl)nc2c1CN(S(=O)(=O)C1CCOCC1)C2)c1cc(N)cc(C(F)(F)F)c1] |
| Example 52. | 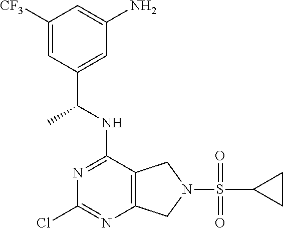 [SMILES: C[C@@H](Nc1nc(Cl)nc2c1CN(S(=O)(=O)C1CC1)C2)c1cc(N)cc(C(F)(F)F)c1] |
| Example 53. | 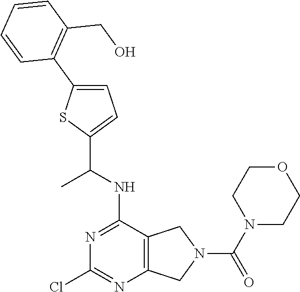 [SMILES: CC(Nc1nc(Cl)nc2c1CN(C(=O)N1CCOCC1)C2)c1ccc(-c2ccccc2CO)s1] |
| Example 54., 110. | 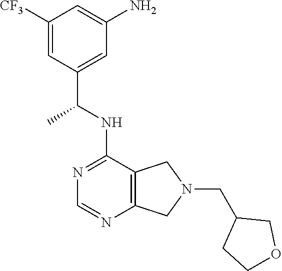 [SMILES: C[C@@H](Nc1ncnc2c1CN(CC1CCOC1)C2)c1cc(N)cc(C(F)(F)F)c1] |
| Example 55. | 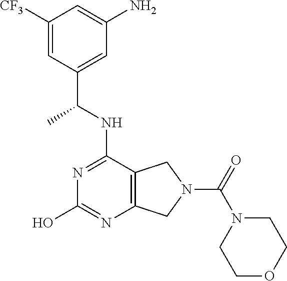 [SMILES: C[C@@H](Nc1nc(O)nc2c1CN(C(=O)N1CCOCC1)C2)c1cc(N)cc(C(F)(F)F)c1] |
| Example 56. | 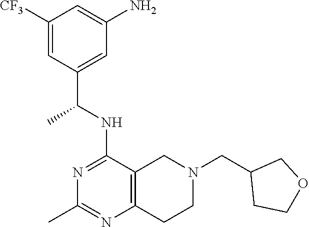 [SMILES: Cc1nc2c(c(N[C@H](C)c3cc(N)cc(C(F)(F)F)c3)n1)CN(CC1CCOC1)CC2] |
| Example 57. | 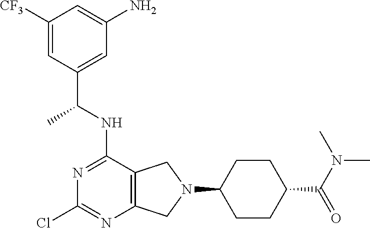 [SMILES: C[C@@H](Nc1nc(Cl)nc2c1CN([C@H]1CC[C@H](C(=O)N(C)C)CC1)C2)c1cc(N)cc(C(F)(F)F)c1] |
| Example 58. | 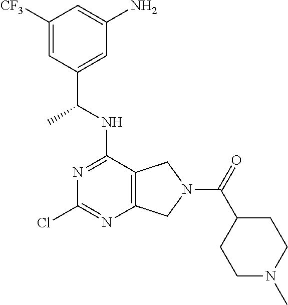 [SMILES: C[C@@H](Nc1nc(Cl)nc2c1CN(C(=O)C1CCN(C)CC1)C2)c1cc(N)cc(C(F)(F)F)c1] |
| Example 59. | 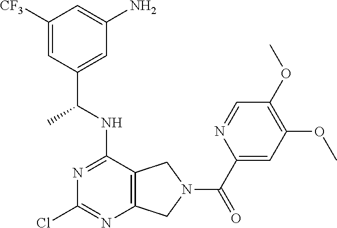 [SMILES: COc1cnc(C(=O)N2Cc3nc(Cl)nc(N[C@H](C)c4cc(N)cc(C(F)(F)F)c4)c3C2)cc1OC] |
| Example 60. | 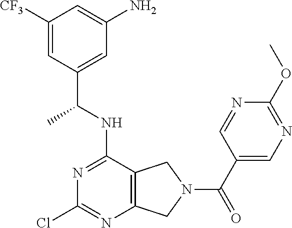 [SMILES: COc1ncc(C(=O)N2Cc3nc(Cl)nc(N[C@H](C)c4cc(N)cc(C(F)(F)F)c4)c3C2)cn1] |
| Example 61. | 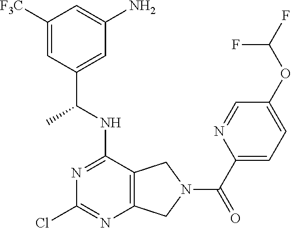 [SMILES: C[C@@H](Nc1nc(Cl)nc2c1CN(C(=O)c1ccc(OC(F)F)cn1)C2)c1cc(N)cc(C(F)(F)F)c1] |
| Example 62. | 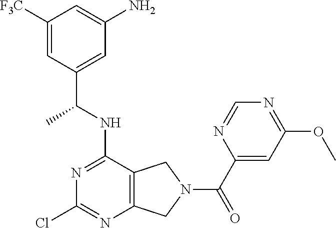 [SMILES: COc1cc(C(=O)N2Cc3nc(Cl)nc(N[C@H](C)c4cc(N)cc(C(F)(F)F)c4)c3C2)ncn1] |
| Example 63. | 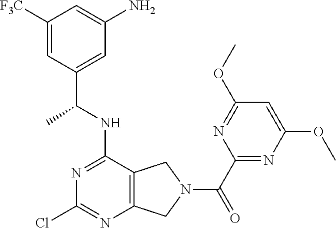 [SMILES: COc1cc(OC)nc(C(=O)N2Cc3nc(Cl)nc(N[C@H](C)c4cc(N)cc(C(F)(F)F)c4)c3C2)n1] |
| Example 64. | 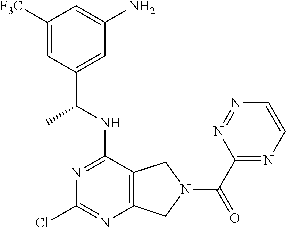 [SMILES: C[C@@H](Nc1nc(Cl)nc2c1CN(C(=O)c1nccnn1)C2)c1cc(N)cc(C(F)(F)F)c1] |
| Example 65. | 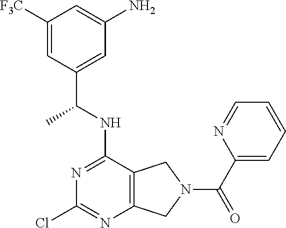 [SMILES: C[C@@H](Nc1nc(Cl)nc2c1CN(C(=O)c1ccccn1)C2)c1cc(N)cc(C(F)(F)F)c1] |
| Example 66. | 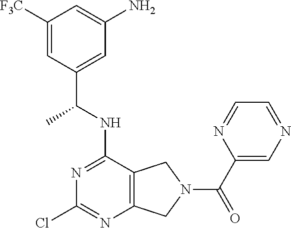 [SMILES: C[C@@H](Nc1nc(Cl)nc2c1CN(C(=O)c1cnccn1)C2)c1cc(N)cc(C(F)(F)F)c1] |
| Example 67. | 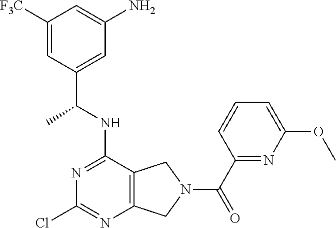 [SMILES: COc1cccc(C(=O)N2Cc3nc(Cl)nc(N[C@H](C)c4cc(N)cc(C(F)(F)F)c4)c3C2)n1] |
| Example 68. | 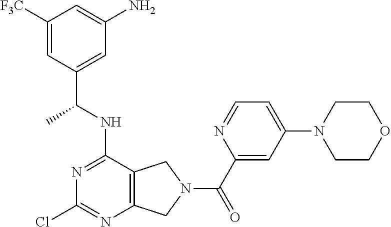 [SMILES: C[C@@H](Nc1nc(Cl)nc2c1CN(C(=O)c1cc(N3CCOCC3)ccn1)C2)c1cc(N)cc(C(F)(F)F)c1] |
| Example 69. | 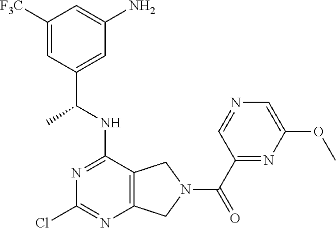 [SMILES: COc1cncc(C(=O)N2Cc3nc(Cl)nc(N[C@H](C)c4cc(N)cc(C(F)(F)F)c4)c3C2)n1] |
| Example 70. | 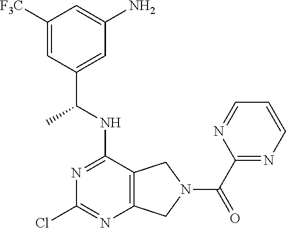 [SMILES: C[C@@H](Nc1nc(Cl)nc2c1CN(C(=O)c1ncccn1)C2)c1cc(N)cc(C(F)(F)F)c1] |
| Example 71. | 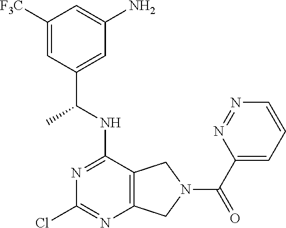 [SMILES: C[C@@H](Nc1nc(Cl)nc2c1CN(C(=O)c1cccnn1)C2)c1cc(N)cc(C(F)(F)F)c1] |
| Example 72. | 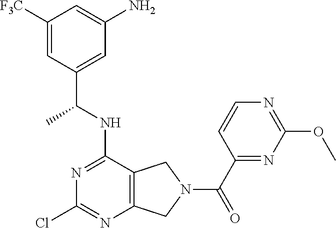 [SMILES: COc1nccc(C(=O)N2Cc3nc(Cl)nc(N[C@H](C)c4cc(N)cc(C(F)(F)F)c4)c3C2)n1] |
| Example 73. | 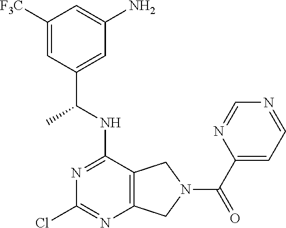 [SMILES: C[C@@H](Nc1nc(Cl)nc2c1CN(C(=O)c1ccncn1)C2)c1cc(N)cc(C(F)(F)F)c1] |
| Example 74. | 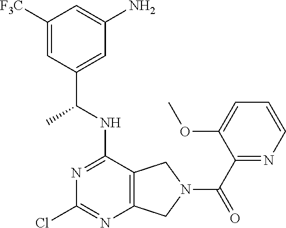 [SMILES: COc1cccnc1C(=O)N1Cc2nc(Cl)nc(N[C@H](C)c3cc(N)cc(C(F)(F)F)c3)c2C1] |
| Example 75. | 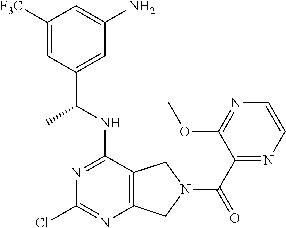 [SMILES: COc1nccnc1C(=O)N1Cc2nc(Cl)nc(N[C@H](C)c3cc(N)cc(C(F)(F)F)c3)c2C1] |
| Example 76. | 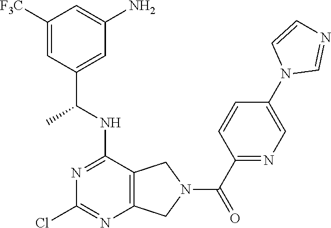 [SMILES: C[C@@H](Nc1nc(Cl)nc2c1CN(C(=O)c1ccc(-n3ccnc3)cn1)C2)c1cc(N)cc(C(F)(F)F)c1] |
| Example 77. | 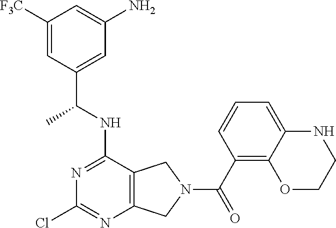 [SMILES: C[C@@H](Nc1nc(Cl)nc2c1CN(C(=O)c1cccc3c1OCCN3)C2)c1cc(N)cc(C(F)(F)F)c1] |
| Example 78. | 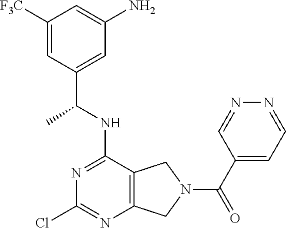 [SMILES: C[C@@H](Nc1nc(Cl)nc2c1CN(C(=O)c1ccnnc1)C2)c1cc(N)cc(C(F)(F)F)c1] |
| Example 79. | 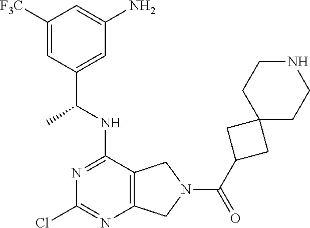 [SMILES: C[C@@H](Nc1nc(Cl)nc2c1CN(C(=O)C1CC3(CCNCC3)C1)C2)c1cc(N)cc(C(F)(F)F)c1] |
| Example 80. | 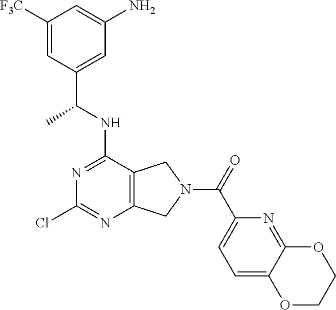 [SMILES: C[C@@H](Nc1nc(Cl)nc2c1CN(C(=O)c1ccc3c(n1)OCCO3)C2)c1cc(N)cc(C(F)(F)F)c1] |
| Example 81. | 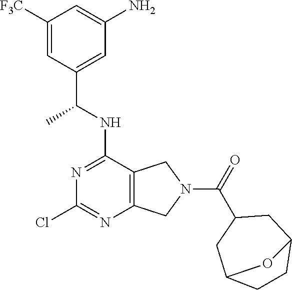 [SMILES: C[C@@H](Nc1nc(Cl)nc2c1CN(C(=O)C1CC3CCC(C1)O3)C2)c1cc(N)cc(C(F)(F)F)c1] |
| Example 82. | 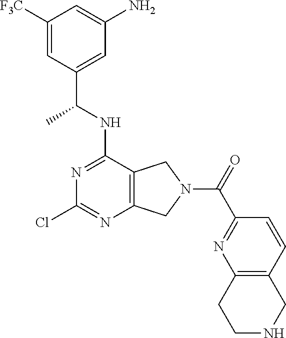 [SMILES: C[C@@H](Nc1nc(Cl)nc2c1CN(C(=O)c1ccc3c(n1)CCNC3)C2)c1cc(N)cc(C(F)(F)F)c1] |
| Example 83. | 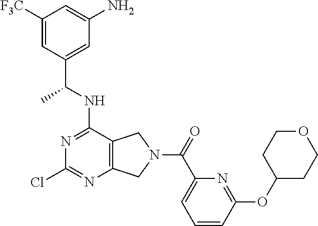 [SMILES: C[C@@H](Nc1nc(Cl)nc2c1CN(C(=O)c1cccc(OC3CCOCC3)n1)C2)c1cc(N)cc(C(F)(F)F)c1] |
| Example 84. | 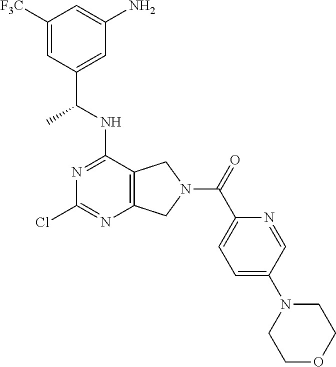 [SMILES: C[C@@H](Nc1nc(Cl)nc2c1CN(C(=O)c1ccc(N3CCOCC3)cn1)C2)c1cc(N)cc(C(F)(F)F)c1] |
| Example 85. | 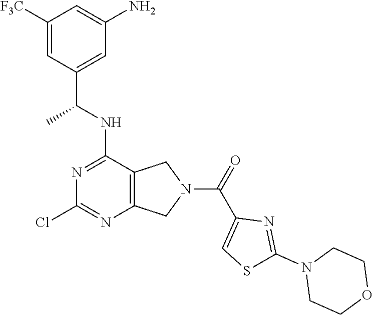 [SMILES: C[C@@H](Nc1nc(Cl)nc2c1CN(C(=O)c1csc(N3CCOCC3)n1)C2)c1cc(N)cc(C(F)(F)F)c1] |
| Example 86. | 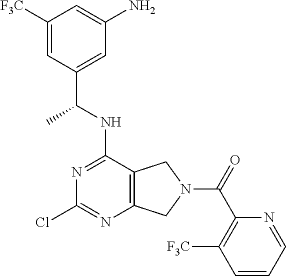 [SMILES: C[C@@H](Nc1nc(Cl)nc2c1CN(C(=O)c1ncccc1C(F)(F)F)C2)c1cc(N)cc(C(F)(F)F)c1] |
| Example 87. | 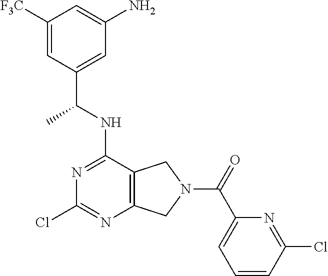 [SMILES: C[C@@H](Nc1nc(Cl)nc2c1CN(C(=O)c1cccc(Cl)n1)C2)c1cc(N)cc(C(F)(F)F)c1] |
| Example 88. | 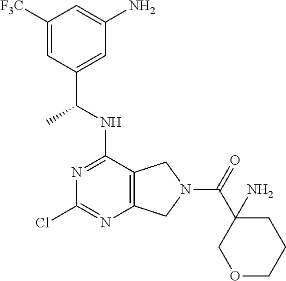 [SMILES: C[C@@H](Nc1nc(Cl)nc2c1CN(C(=O)C1(N)CCCOC1)C2)c1cc(N)cc(C(F)(F)F)c1] |
| Example 89. | 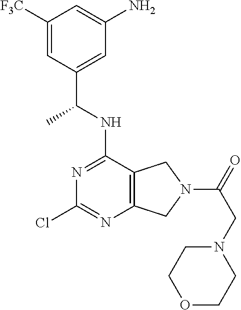 [SMILES: C[C@@H](Nc1nc(Cl)nc2c1CN(C(=O)CN1CCOCC1)C2)c1cc(N)cc(C(F)(F)F)c1] |
| Example 90. | 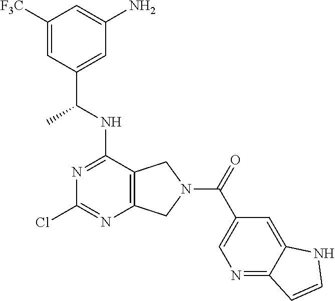 [SMILES: C[C@@H](Nc1nc(Cl)nc2c1CN(C(=O)c1cnc3cc[nH]c3c1)C2)c1cc(N)cc(C(F)(F)F)c1] |
| Example 91. | 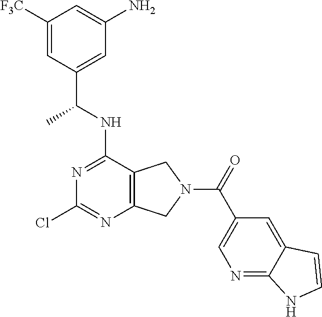 [SMILES: C[C@@H](Nc1nc(Cl)nc2c1CN(C(=O)c1cnc3[nH]ccc3c1)C2)c1cc(N)cc(C(F)(F)F)c1] |
| Example 92. | 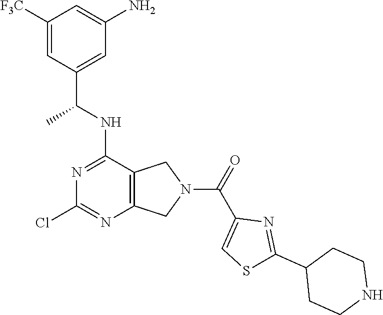 [SMILES: C[C@@H](Nc1nc(Cl)nc2c1CN(C(=O)c1csc(C3CCNCC3)n1)C2)c1cc(N)cc(C(F)(F)F)c1] |
| Example 93. | 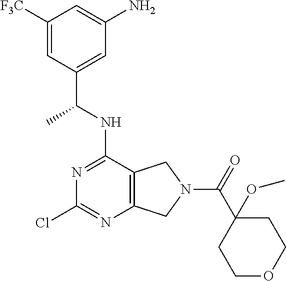 [SMILES: COC1(C(=O)N2Cc3nc(Cl)nc(N[C@H](C)c4cc(N)cc(C(F)(F)F)c4)c3C2)CCOCC1] |
| Example 94. | 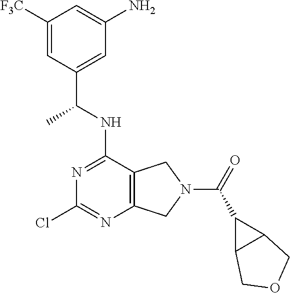 |
| Example 95. | 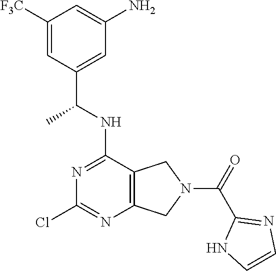 [SMILES: C[C@@H](Nc1nc(Cl)nc2c1CN(C(=O)c1ncc[nH]1)C2)c1cc(N)cc(C(F)(F)F)c1] |
| Example 96. | 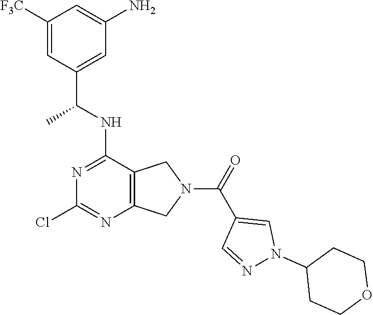 [SMILES: C[C@@H](Nc1nc(Cl)nc2c1CN(C(=O)c1cnn(C3CCOCC3)c1)C2)c1cc(N)cc(C(F)(F)F)c1] |
| Example 97. | 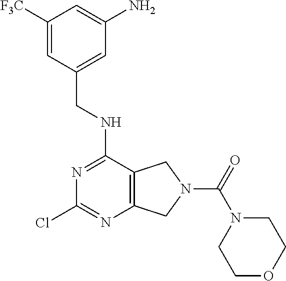 [SMILES: Nc1cc(CNc2nc(Cl)nc3c2CN(C(=O)N2CCOCC2)C3)cc(C(F)(F)F)c1] |
| Example 98. | 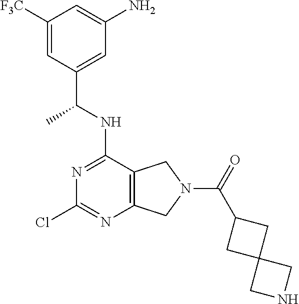 [SMILES: C[C@@H](Nc1nc(Cl)nc2c1CN(C(=O)C1CC3(CNC3)C1)C2)c1cc(N)cc(C(F)(F)F)c1] |
| Example 99. | 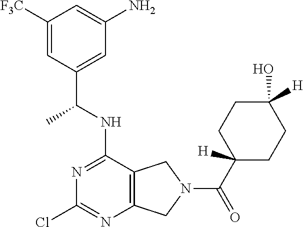 [SMILES: C[C@@H](Nc1nc(Cl)nc2c1CN(C(=O)[C@H]1CC[C@@H](O)CC1)C2)c1cc(N)cc(C(F)(F)F)c1] |
| Example 100. | 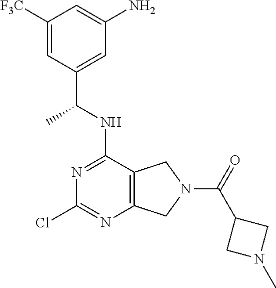 [SMILES: C[C@@H](Nc1nc(Cl)nc2c1CN(C(=O)C1CN(C)C1)C2)c1cc(N)cc(C(F)(F)F)c1] |
| Example 101. | 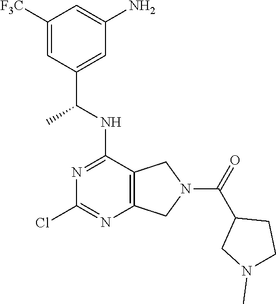 [SMILES: C[C@@H](Nc1nc(Cl)nc2c1CN(C(=O)C1CCN(C)C1)C2)c1cc(N)cc(C(F)(F)F)c1] |
| Example 102. | 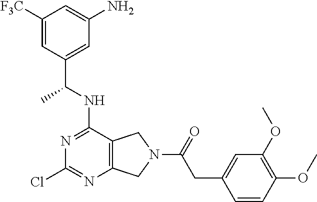 [SMILES: COc1ccc(CC(=O)N2Cc3nc(Cl)nc(N[C@H](C)c4cc(N)cc(C(F)(F)F)c4)c3C2)cc1OC] |
| Example 103. | 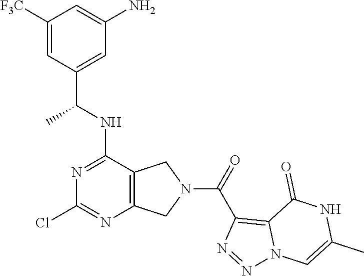 [SMILES: Cc1cn2nnc(C(=O)N3Cc4nc(Cl)nc(N[C@H](C)c5cc(N)cc(C(F)(F)F)c5)c4C3)c2c(=O)[nH]1] |
| Example 104. | 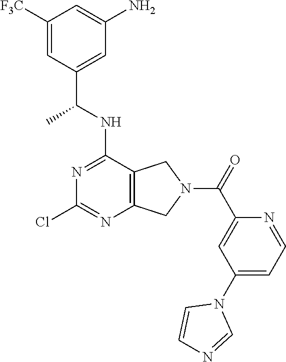 [SMILES: C[C@@H](Nc1nc(Cl)nc2c1CN(C(=O)c1cc(-n3ccnc3)ccn1)C2)c1cc(N)cc(C(F)(F)F)c1] |
| Example 105. | 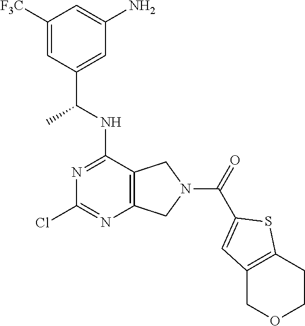 [SMILES: C[C@@H](Nc1nc(Cl)nc2c1CN(C(=O)c1cc3c(s1)CCOC3)C2)c1cc(N)cc(C(F)(F)F)c1] |
| Example 106. | 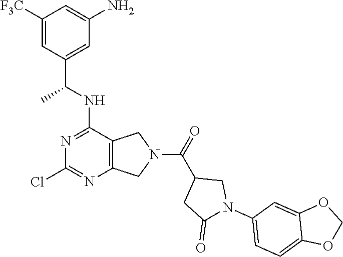 [SMILES: C[C@@H](Nc1nc(Cl)nc2c1CN(C(=O)C1CC(=O)N(c3ccc4c(c3)OCO4)C1)C2)c1cc(N)cc(C(F)(F)F)c1] |
| Example 107. | 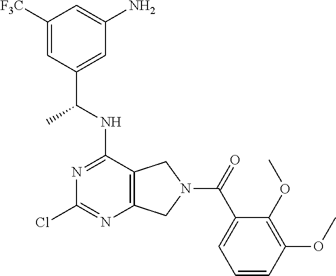 [SMILES: COc1cccc(C(=O)N2Cc3nc(Cl)nc(N[C@H](C)c4cc(N)cc(C(F)(F)F)c4)c3C2)c1OC] |
| Example 108. | 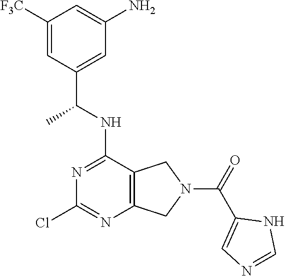 [SMILES: C[C@@H](Nc1nc(Cl)nc2c1CN(C(=O)c1cnc[nH]1)C2)c1cc(N)cc(C(F)(F)F)c1] |
| Example 109. | 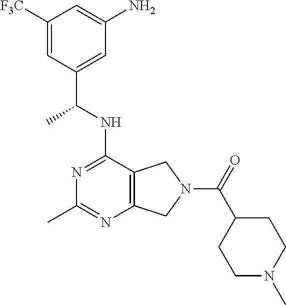 [SMILES: Cc1nc2c(c(N[C@H](C)c3cc(N)cc(C(F)(F)F)c3)n1)CN(C(=O)C1CCN(C)CC1)C2] |
| Example 111. | 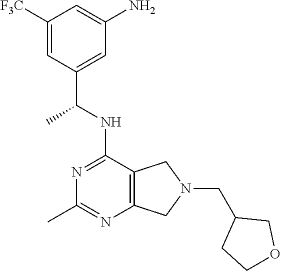 [SMILES: Cc1nc2c(c(N[C@H](C)c3cc(N)cc(C(F)(F)F)c3)n1)CN(CC1CCOC1)C2] |
| Example 112. | 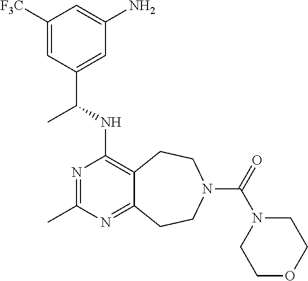 [SMILES: Cc1nc2c(c(N[C@H](C)c3cc(N)cc(C(F)(F)F)c3)n1)CCN(C(=O)N1CCOCC1)CC2] |
| Example 113. | 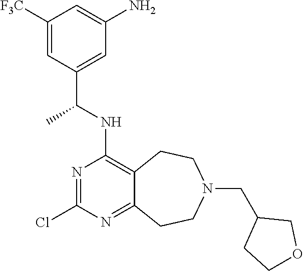 [SMILES: C[C@@H](Nc1nc(Cl)nc2c1CCN(CC1CCOC1)CC2)c1cc(N)cc(C(F)(F)F)c1] |
| Example 114. | 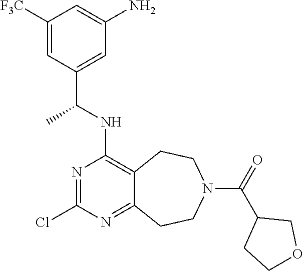 [SMILES: C[C@@H](Nc1nc(Cl)nc2c1CCN(C(=O)C1CCOC1)CC2)c1cc(N)cc(C(F)(F)F)c1] |
| Example 115. | 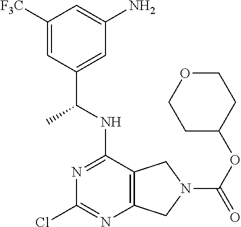 [SMILES: C[C@@H](Nc1nc(Cl)nc2c1CN(C(=O)OC1CCOCC1)C2)c1cc(N)cc(C(F)(F)F)c1] |
| Example 116. | 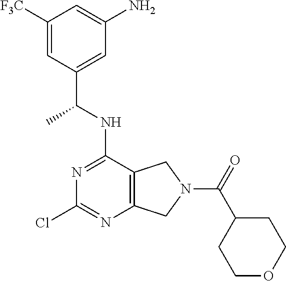 [SMILES: C[C@@H](Nc1nc(Cl)nc2c1CN(C(=O)C1CCOCC1)C2)c1cc(N)cc(C(F)(F)F)c1] |
| Example 117. | 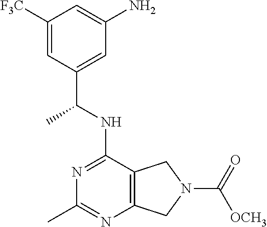 [SMILES: COC(=O)N1Cc2nc(C)nc(N[C@H](C)c3cc(N)cc(C(F)(F)F)c3)c2C1] |
| Example 118. | 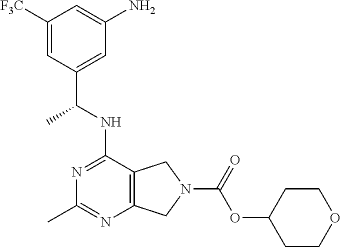 [SMILES: Cc1nc2c(c(N[C@H](C)c3cc(N)cc(C(F)(F)F)c3)n1)CN(C(=O)OC1CCOCC1)C2] |
| Example 119. | 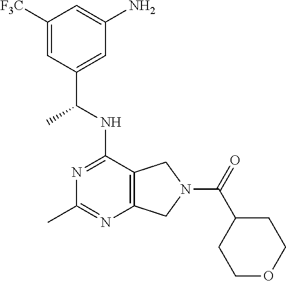 [SMILES: Cc1nc2c(c(N[C@H](C)c3cc(N)cc(C(F)(F)F)c3)n1)CN(C(=O)C1CCOCC1)C2] |
| Example 120. | 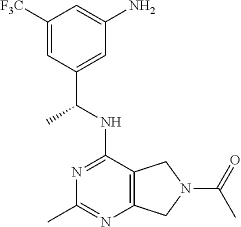 [SMILES: CC(=O)N1Cc2nc(C)nc(N[C@H](C)c3cc(N)cc(C(F)(F)F)c3)c2C1] |
| Example 121. | 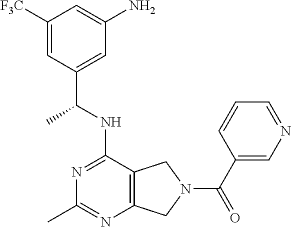 [SMILES: Cc1nc2c(c(N[C@H](C)c3cc(N)cc(C(F)(F)F)c3)n1)CN(C(=O)c1cccnc1)C2] |
| Example 122. | 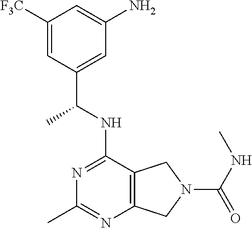 [SMILES: CNC(=O)N1Cc2nc(C)nc(N[C@H](C)c3cc(N)cc(C(F)(F)F)c3)c2C1] |
| Example 123. | 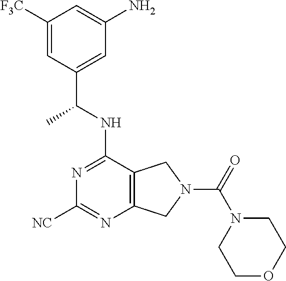 [SMILES: C[C@@H](Nc1nc(C#N)nc2c1CN(C(=O)N1CCOCC1)C2)c1cc(N)cc(C(F)(F)F)c1] |
| Example 124. | 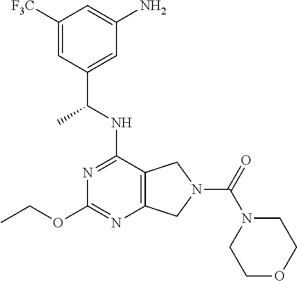 [SMILES: CCOc1nc2c(c(N[C@H](C)c3cc(N)cc(C(F)(F)F)c3)n1)CN(C(=O)N1CCOCC1)C2] |
| Example 125. | 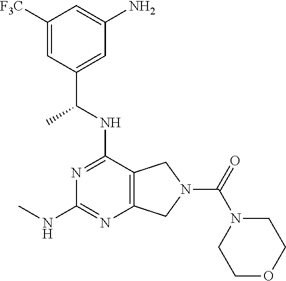 [SMILES: CNc1nc2c(c(N[C@H](C)c3cc(N)cc(C(F)(F)F)c3)n1)CN(C(=O)N1CCOCC1)C2] |
| Example 126. | 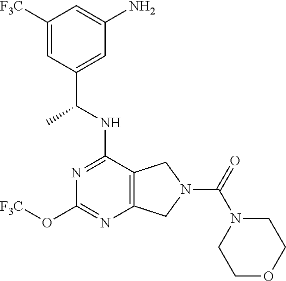 [SMILES: C[C@@H](Nc1nc(OC(F)(F)F)nc2c1CN(C(=O)N1CCOCC1)C2)c1cc(N)cc(C(F)(F)F)c1] |
| Example 127. | 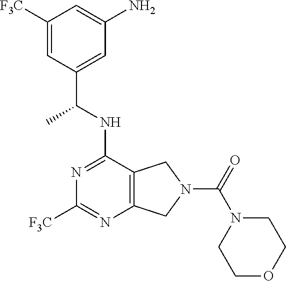 [SMILES: C[C@@H](Nc1nc(C(F)(F)F)nc2c1CN(C(=O)N1CCOCC1)C2)c1cc(N)cc(C(F)(F)F)c1] |
| Example 128. | 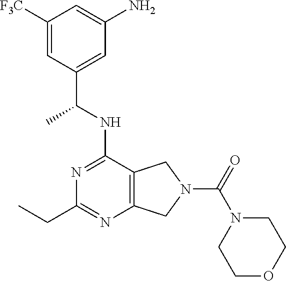 [SMILES: CCc1nc2c(c(N[C@H](C)c3cc(N)cc(C(F)(F)F)c3)n1)CN(C(=O)N1CCOCC1)C2] |
| Example 129. | 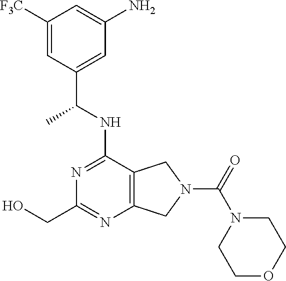 [SMILES: C[C@@H](Nc1nc(CO)nc2c1CN(C(=O)N1CCOCC1)C2)c1cc(N)cc(C(F)(F)F)c1] |
| Example 130. | 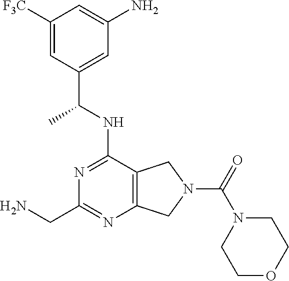 [SMILES: C[C@@H](Nc1nc(CN)nc2c1CN(C(=O)N1CCOCC1)C2)c1cc(N)cc(C(F)(F)F)c1] |
| Example 131. | 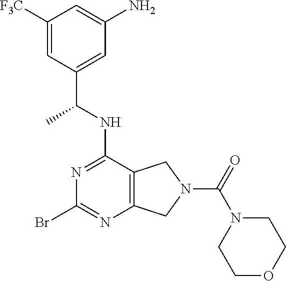 [SMILES: C[C@@H](Nc1nc(Br)nc2c1CN(C(=O)N1CCOCC1)C2)c1cc(N)cc(C(F)(F)F)c1] |
| Example 132. | 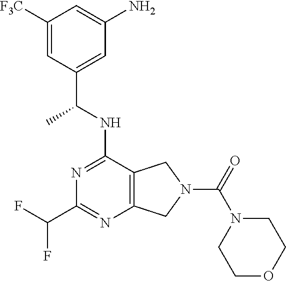 [SMILES: C[C@@H](Nc1nc(C(F)F)nc2c1CN(C(=O)N1CCOCC1)C2)c1cc(N)cc(C(F)(F)F)c1] |
| Example 133. | 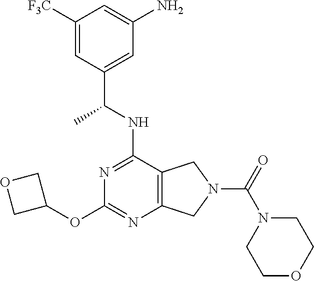 [SMILES: C[C@@H](Nc1nc(OC2COC2)nc2c1CN(C(=O)N1CCOCC1)C2)c1cc(N)cc(C(F)(F)F)c1] |
| Example 134. | 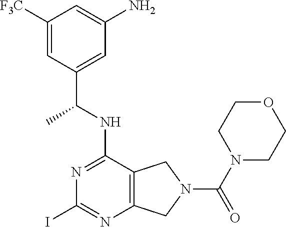 [SMILES: C[C@@H](Nc1nc(I)nc2c1CN(C(=O)N1CCOCC1)C2)c1cc(N)cc(C(F)(F)F)c1] |
| Example 135. | 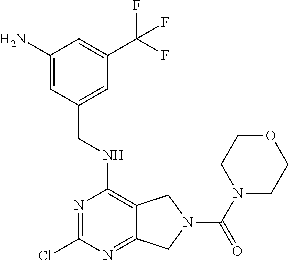 [SMILES: Nc1cc(CNc2nc(Cl)nc3c2CN(C(=O)N2CCOCC2)C3)cc(C(F)(F)F)c1] |
| Example 136. | 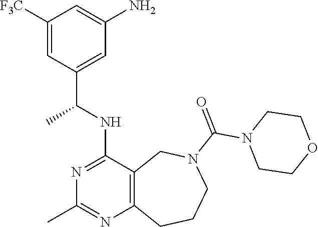 [SMILES: Cc1nc2c(c(N[C@H](C)c3cc(N)cc(C(F)(F)F)c3)n1)CN(C(=O)N1CCOCC1)CCC2] |
| Example 137. | 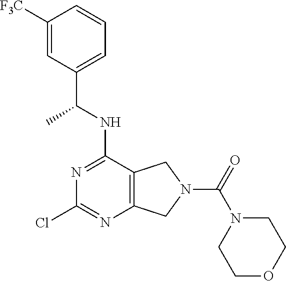 [SMILES: C[C@@H](Nc1nc(Cl)nc2c1CN(C(=O)N1CCOCC1)C2)c1cccc(C(F)(F)F)c1] |
| Example 138. | 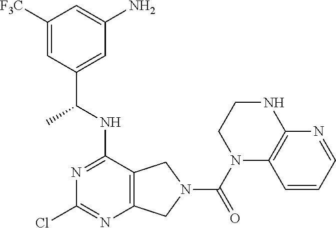 [SMILES: C[C@@H](Nc1nc(Cl)nc2c1CN(C(=O)N1CCNc3ncccc31)C2)c1cc(N)cc(C(F)(F)F)c1] |
| Example 139. | 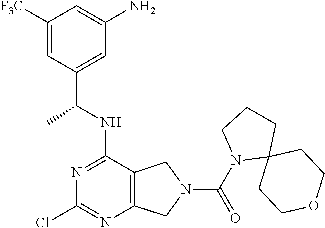 [SMILES: C[C@@H](Nc1nc(Cl)nc2c1CN(C(=O)N1CCCC13CCOCC3)C2)c1cc(N)cc(C(F)(F)F)c1] |
| Example 140. | 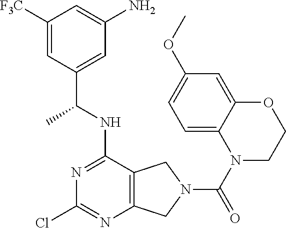 [SMILES: COc1ccc2c(c1)OCCN2C(=O)N1Cc2nc(Cl)nc(N[C@H](C)c3cc(N)cc(C(F)(F)F)c3)c2C1] |
| Example 141. | 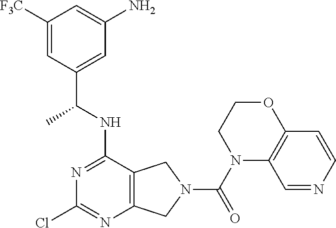 [SMILES: C[C@@H](Nc1nc(Cl)nc2c1CN(C(=O)N1CCOc3ccncc31)C2)c1cc(N)cc(C(F)(F)F)c1] |
| Example 142. | 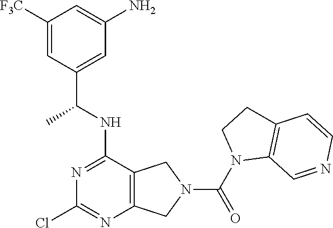 [SMILES: C[C@@H](Nc1nc(Cl)nc2c1CN(C(=O)N1CCc3ccncc31)C2)c1cc(N)cc(C(F)(F)F)c1] |
| Example 143. | 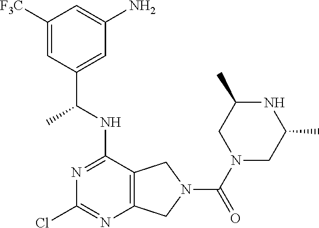 [SMILES: C[C@@H]1CN(C(=O)N2Cc3nc(Cl)nc(N[C@H](C)c4cc(N)cc(C(F)(F)F)c4)c3C2)C[C@@H](C)N1] |
| Example 144. | 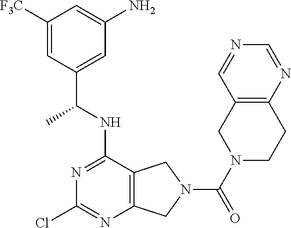 [SMILES: C[C@@H](Nc1nc(Cl)nc2c1CN(C(=O)N1CCc3ncncc3C1)C2)c1cc(N)cc(C(F)(F)F)c1] |
| Example 145. | 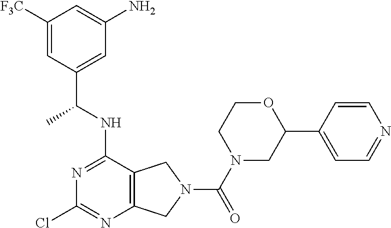 [SMILES: C[C@@H](Nc1nc(Cl)nc2c1CN(C(=O)N1CCOC(c3ccncc3)C1)C2)c1cc(N)cc(C(F)(F)F)c1] |
| Example 146. | 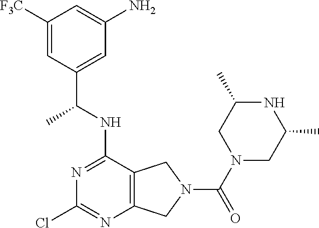 [SMILES: C[C@@H]1CN(C(=O)N2Cc3nc(Cl)nc(N[C@H](C)c4cc(N)cc(C(F)(F)F)c4)c3C2)C[C@H](C)N1] |
| Example 147. | 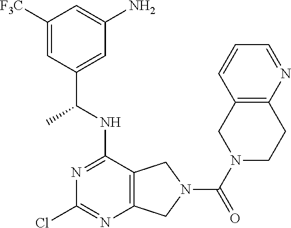 [SMILES: C[C@@H](Nc1nc(Cl)nc2c1CN(C(=O)N1CCc3ncccc3C1)C2)c1cc(N)cc(C(F)(F)F)c1] |
| Example 148. | 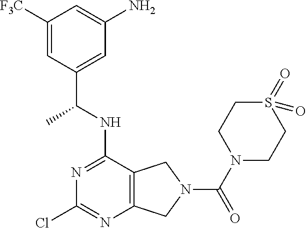 [SMILES: C[C@@H](Nc1nc(Cl)nc2c1CN(C(=O)N1CCS(=O)(=O)CC1)C2)c1cc(N)cc(C(F)(F)F)c1] |
| Example 149. | 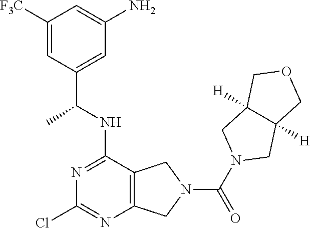 [SMILES: C[C@@H](Nc1nc(Cl)nc2c1CN(C(=O)N1C[C@H]3COC[C@H]3C1)C2)c1cc(N)cc(C(F)(F)F)c1] |
| Example 150. | 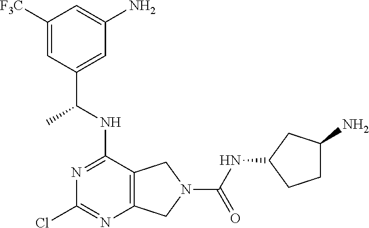 |
| Example 151. | 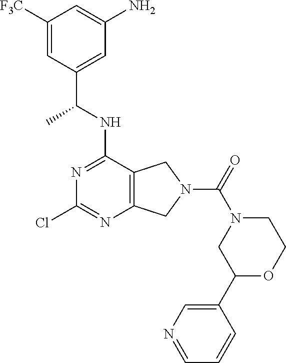 [SMILES: C[C@@H](Nc1nc(Cl)nc2c1CN(C(=O)N1CCOC(c3cccnc3)C1)C2)c1cc(N)cc(C(F)(F)F)c1] |
| Example 152. | 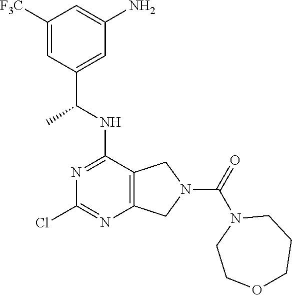 [SMILES: C[C@@H](Nc1nc(Cl)nc2c1CN(C(=O)N1CCCOCC1)C2)c1cc(N)cc(C(F)(F)F)c1] |
| Example 153. | 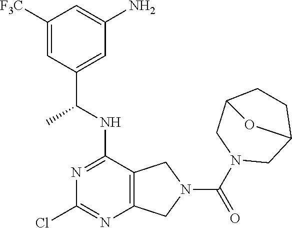 [SMILES: C[C@@H](Nc1nc(Cl)nc2c1CN(C(=O)N1CC3CCC(C1)O3)C2)c1cc(N)cc(C(F)(F)F)c1] |
| Example 154. | 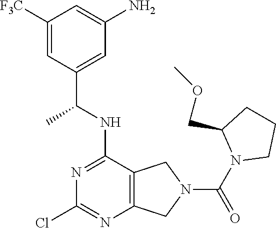 [SMILES: COC[C@H]1CCCN1C(=O)N1Cc2nc(Cl)nc(N[C@H](C)c3cc(N)cc(C(F)(F)F)c3)c2C1] |
| Example 155. | 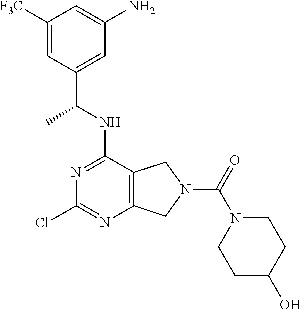 [SMILES: C[C@@H](Nc1nc(Cl)nc2c1CN(C(=O)N1CCC(O)CC1)C2)c1cc(N)cc(C(F)(F)F)c1] |
| Example 156. | 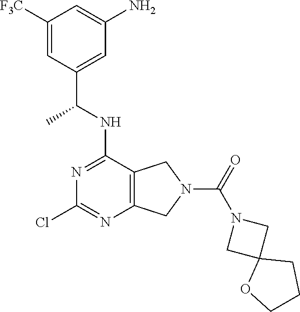 [SMILES: C[C@@H](Nc1nc(Cl)nc2c1CN(C(=O)N1CC3(CCCO3)C1)C2)c1cc(N)cc(C(F)(F)F)c1] |
| Example 157. | 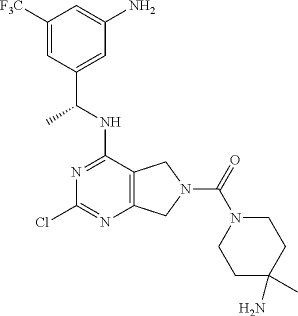 [SMILES: C[C@@H](Nc1nc(Cl)nc2c1CN(C(=O)N1CCC(C)(N)CC1)C2)c1cc(N)cc(C(F)(F)F)c1] |
| Example 158. | 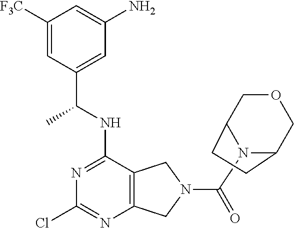 [SMILES: C[C@@H](Nc1nc(Cl)nc2c1CN(C(=O)N1C3CCC1COC3)C2)c1cc(N)cc(C(F)(F)F)c1] |
| Example 159. | 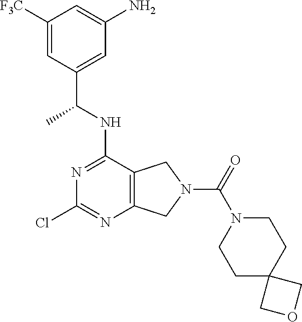 [SMILES: C[C@@H](Nc1nc(Cl)nc2c1CN(C(=O)N1CCC3(CC1)COC3)C2)c1cc(N)cc(C(F)(F)F)c1] |
| Example 160. | 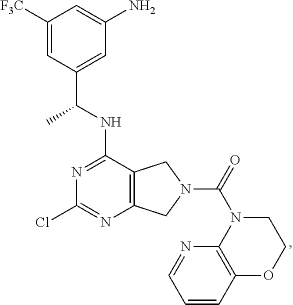 [SMILES: C[C@@H](Nc1nc(Cl)nc2c1CN(C(=O)N1CCOc3cccnc31)C2)c1cc(N)cc(C(F)(F)F)c1] |
| Example 161. | 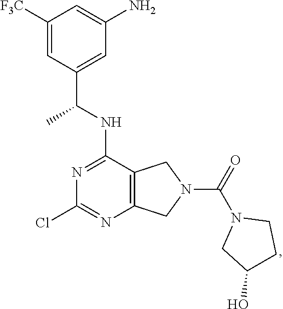 [SMILES: C[C@@H](Nc1nc(Cl)nc2c1CN(C(=O)N1CC[C@H](O)C1)C2)c1cc(N)cc(C(F)(F)F)c1] |
| Example 162. | 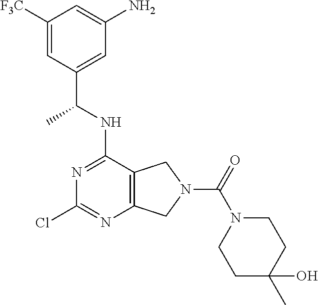 [SMILES: C[C@@H](Nc1nc(Cl)nc2c1CN(C(=O)N1CCC(C)(O)CC1)C2)c1cc(N)cc(C(F)(F)F)c1] |
| Example 163. | 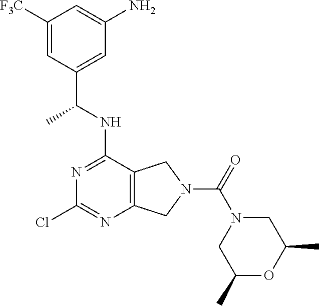 [SMILES: C[C@@H]1CN(C(=O)N2Cc3nc(Cl)nc(N[C@H](C)c4cc(N)cc(C(F)(F)F)c4)c3C2)C[C@H](C)O1] |
| Example 164. | 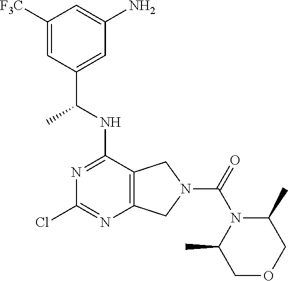 [SMILES: C[C@@H]1COC[C@H](C)N1C(=O)N1Cc2nc(Cl)nc(N[C@H](C)c3cc(N)cc(C(F)(F)F)c3)c2C1] |
| Example 165. | 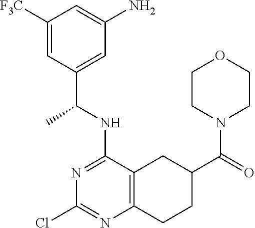 [SMILES: C[C@@H](Nc1nc(Cl)nc2c1CC(C(=O)N1CCOCC1)CC2)c1cc(N)cc(C(F)(F)F)c1] |
| Example 167. | 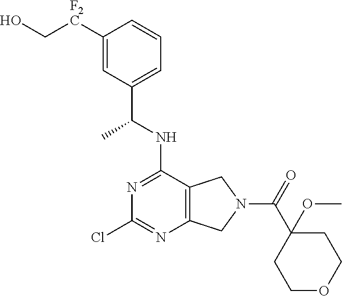 [SMILES: COC1(C(=O)N2Cc3nc(Cl)nc(N[C@H](C)c4cccc(C(F)(F)CO)c4)c3C2)CCOCC1] |
| Example 168. | 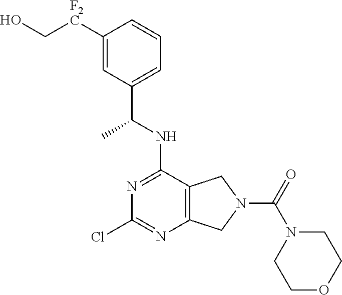 [SMILES: C[C@@H](Nc1nc(Cl)nc2c1CN(C(=O)N1CCOCC1)C2)c1cccc(C(F)(F)CO)c1] |
| Example 169. | 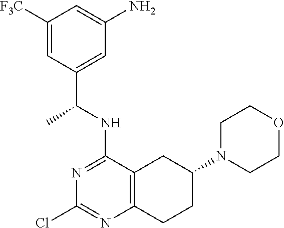 [SMILES: C[C@@H](Nc1nc(Cl)nc2c1C[C@H](N1CCOCC1)CC2)c1cc(N)cc(C(F)(F)F)c1] |
| Example 170. | 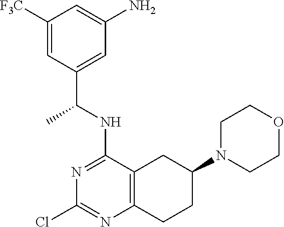 [SMILES: C[C@@H](Nc1nc(Cl)nc2c1C[C@@H](N1CCOCC1)CC2)c1cc(N)cc(C(F)(F)F)c1] |
| Example 171. | 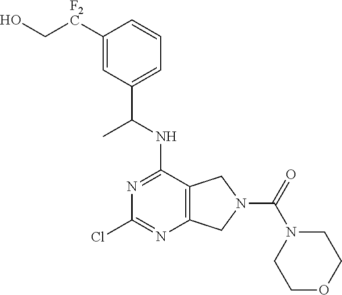 [SMILES: CC(Nc1nc(Cl)nc2c1CN(C(=O)N1CCOCC1)C2)c1cccc(C(F)(F)CO)c1] |
| Example 172. | 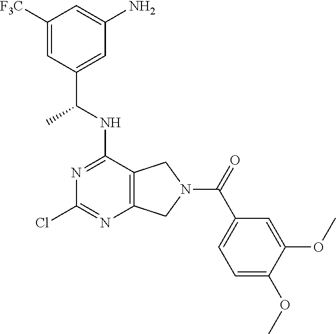 [SMILES: COc1ccc(C(=O)N2Cc3nc(Cl)nc(N[C@H](C)c4cc(N)cc(C(F)(F)F)c4)c3C2)cc1OC] |
| Example 173. | 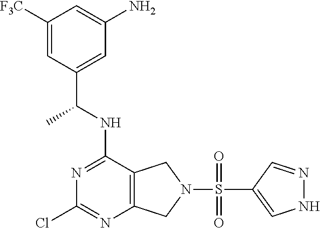 [SMILES: C[C@@H](Nc1nc(Cl)nc2c1CN(S(=O)(=O)c1cn[nH]c1)C2)c1cc(N)cc(C(F)(F)F)c1] |
| Example 174. | 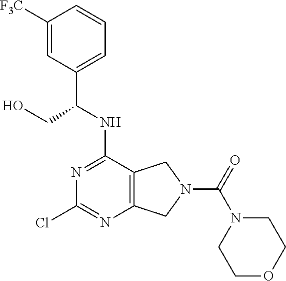 [SMILES: O=C(N1CCOCC1)N1Cc2nc(Cl)nc(N[C@H](CO)c3cccc(C(F)(F)F)c3)c2C1] |
| Example 175. | 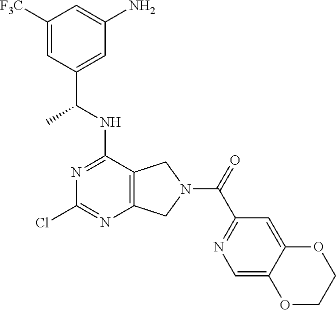 [SMILES: C[C@@H](Nc1nc(Cl)nc2c1CN(C(=O)c1cc3c(cn1)OCCO3)C2)c1cc(N)cc(C(F)(F)F)c1] |
| Example 176. | 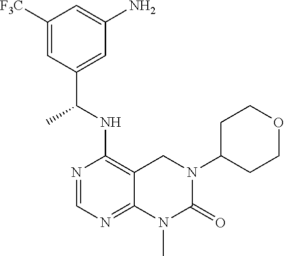 [SMILES: C[C@@H](Nc1ncnc2c1CN(C1CCOCC1)C(=O)N2C)c1cc(N)cc(C(F)(F)F)c1] |
| Example 177. | 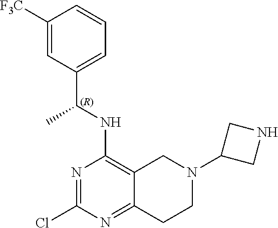 |
| Example 178. | 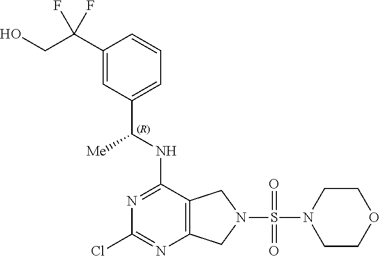 |
| Example 179. | 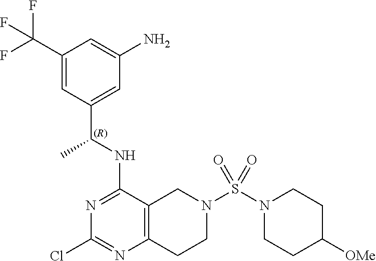 |
| Example 180. | 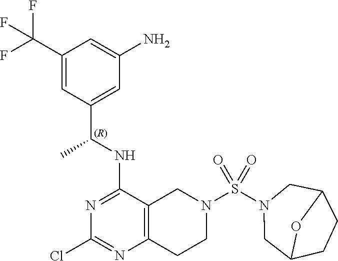 |
| Example 181. | 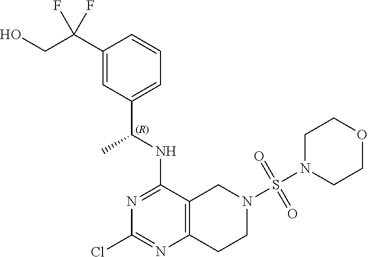 |
| Example 182. | 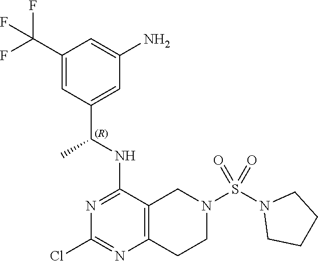 |
| Example 183. | 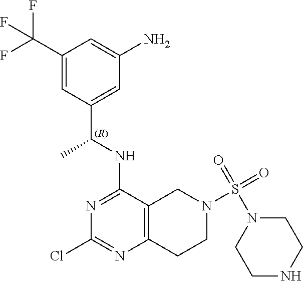 |
| Example 184. | 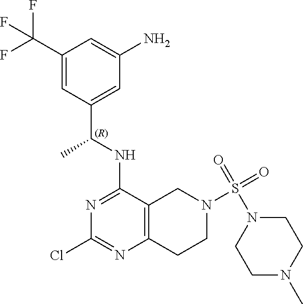 |
| Example 185. | 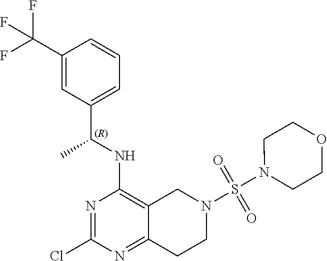 |
| Example 186. | 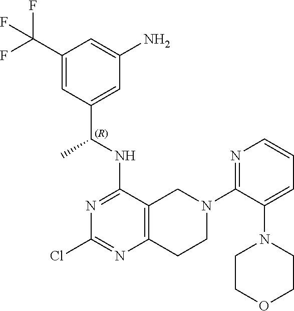 |
| Example 187. | 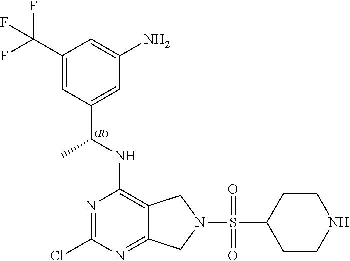 |
| Example 188. | 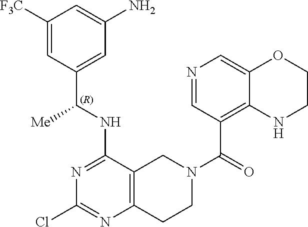 |
| Example 189. | 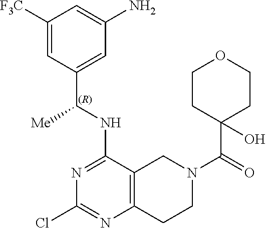 |
| Example 190. | 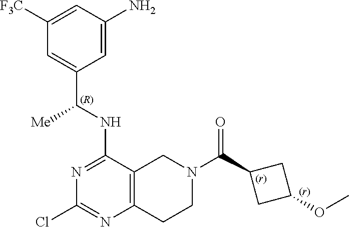 |
| Example 191. | 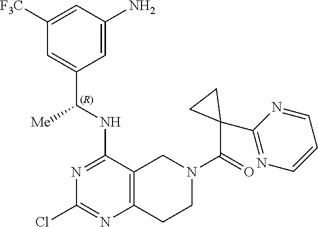 |
| Example 192. | 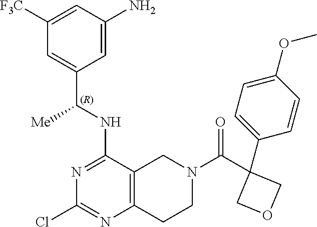 |
| Example 193. | 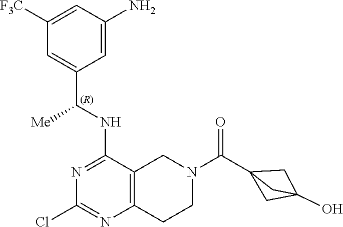 |
| Example 194. | 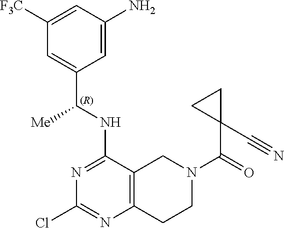 |
| Example 195. | 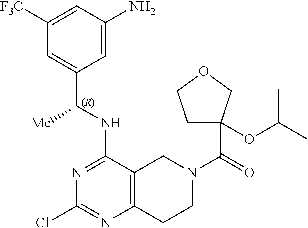 |
| Example 196. | 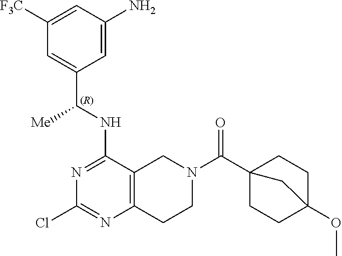 |
| Example 197. | 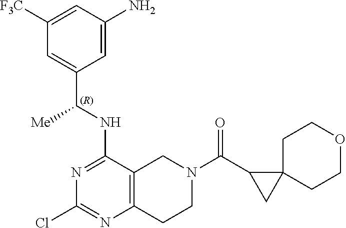 |
| Example 198. | 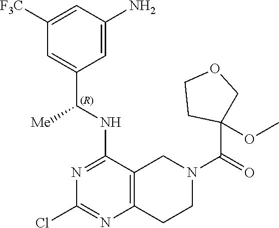 |
| Example 199. | 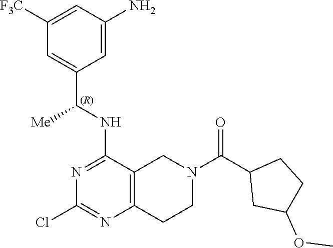 |
| Example 200. | 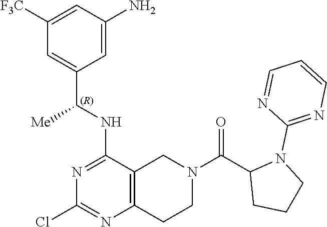 |
| Example 201. | 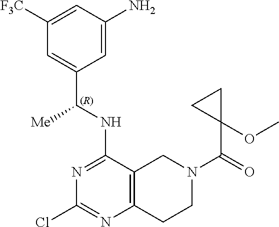 |
| Example 202. | 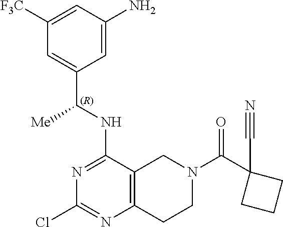 |
| Example 203. | 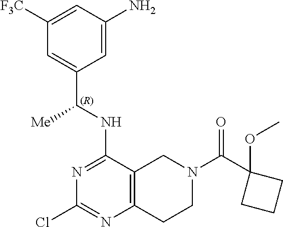 |
| Example 204. | 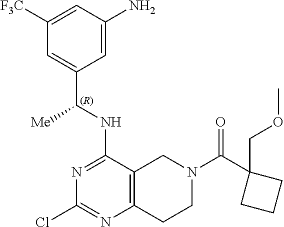 |
| Example 205. | 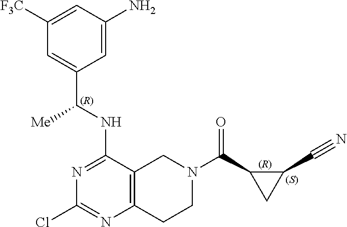 |
| Example 206. | 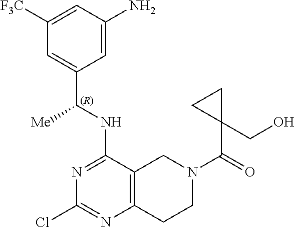 |
| Example 207. | 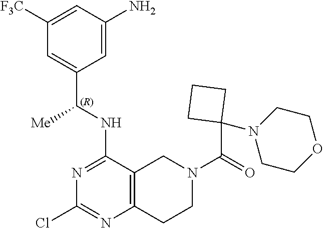 |
| Example 208. | 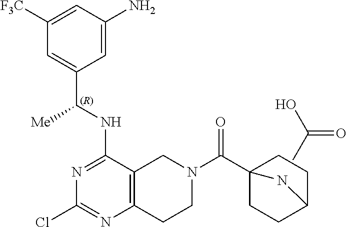 |
| Example 209. | 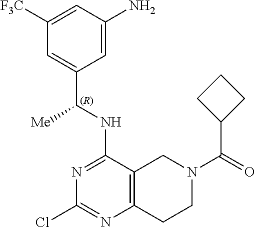 |
| Example 210. | 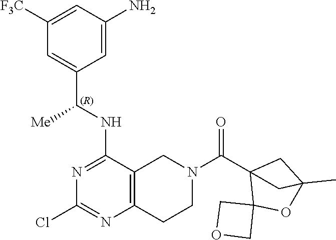 |
| Example 211. | 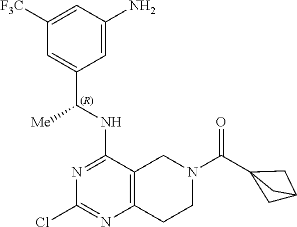 |
| Example 212. | 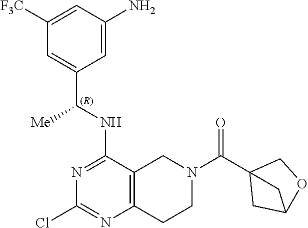 |
| Example 213. | 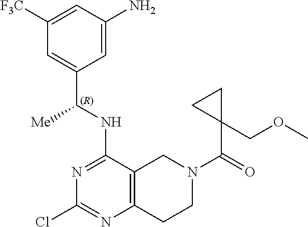 |
| Example 214. | 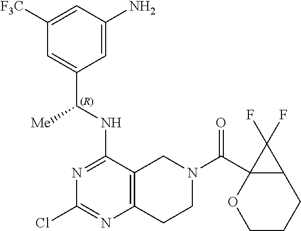 |
| Example 215. | 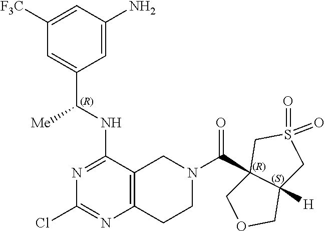 |
| Example 216. | 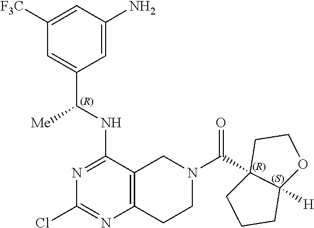 |
| Example 217. | 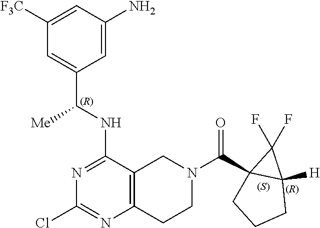 |
| Example 218. | 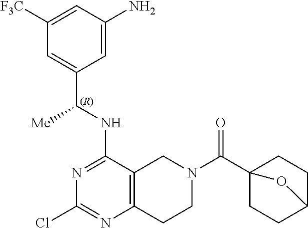 |
| Example 219. | 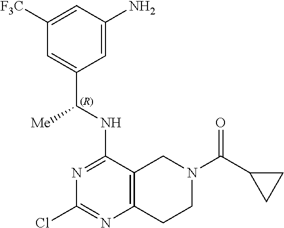 |
| Example 220. | 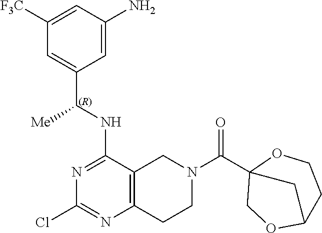 |
| Example 221. | 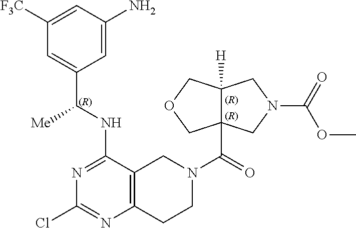 |
| Example 222. | 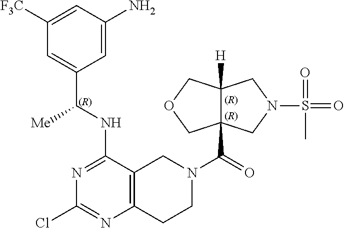 |
| Example 223. | 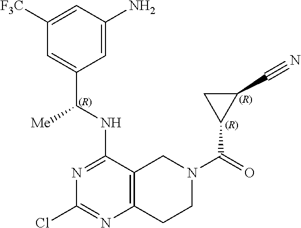 |
| Example 224. | 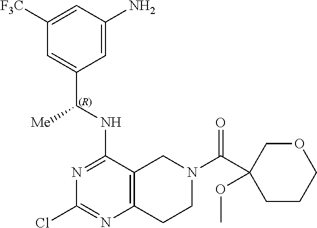 |
| Example 225. | 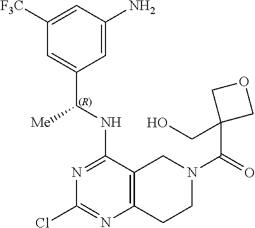 |
| Example 226. | 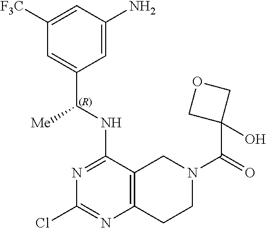 |
| Example 227. | 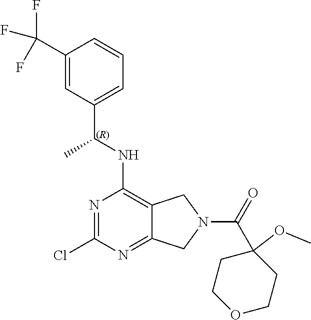 |
| Example 228. | 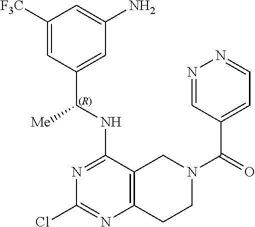 |
| Example 229. | 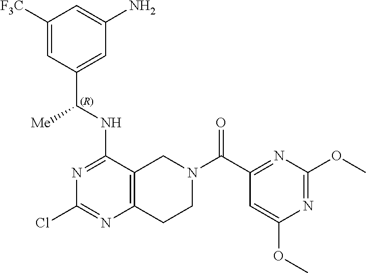 |
| Example 230. | 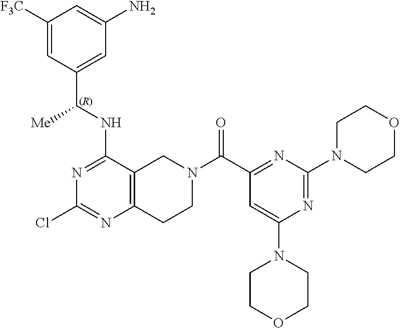 |
| Example 231. | 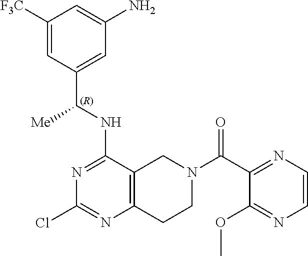 |
| Example 232. | 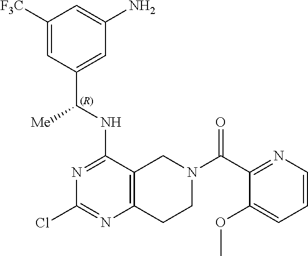 |
| Example 233. | 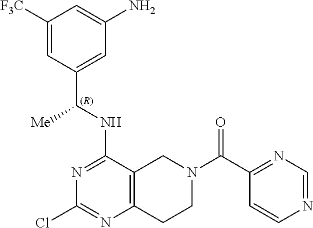 |
| Example 234. | 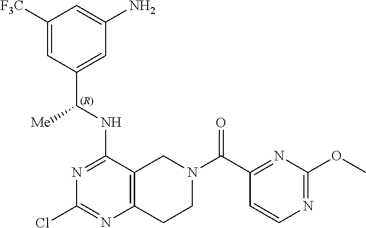 |
| Example 235. | 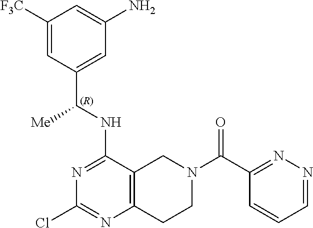 |
| Example 236. | 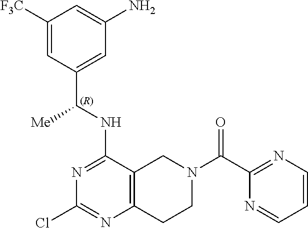 |
| Example 237. | 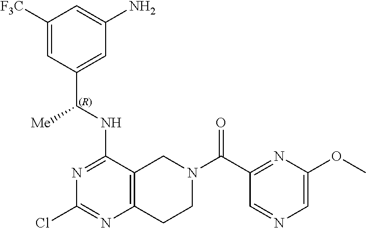 |
| Example 238. | 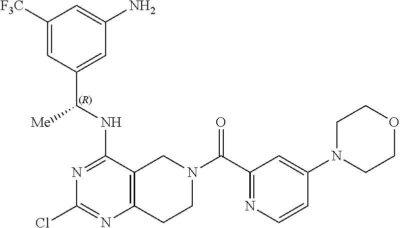 |
| Example 239. | 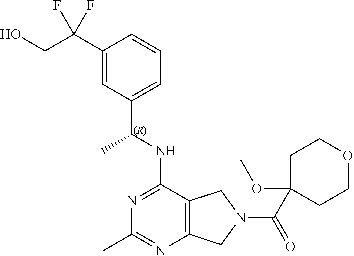 |
| Example 240. | 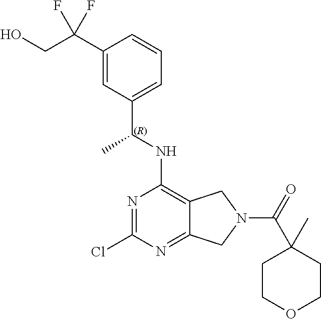 |
| Example 241. | 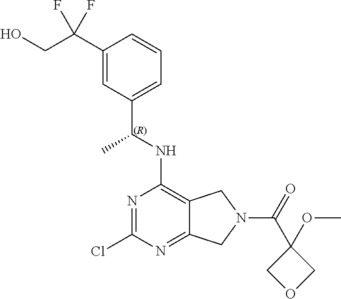 |
| Example 242. | 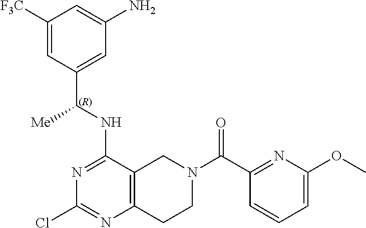 |
| Example 243. | 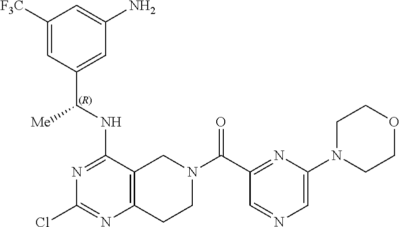 |
| Example 244. | 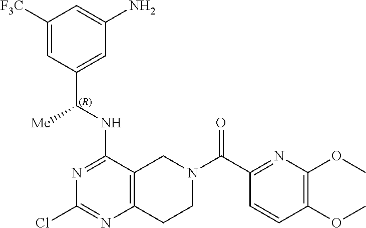 |
| Example 245. | 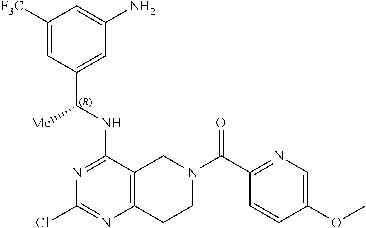 |
| Example 246. | 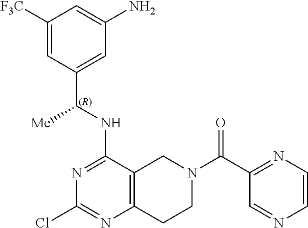 |
| Example 247. | 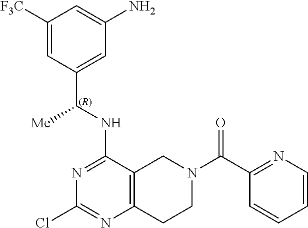 |
| Example 248. | 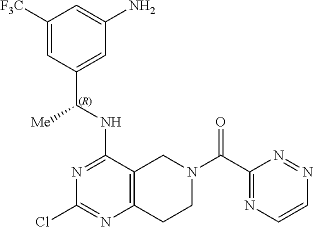 |
| Example 249. | 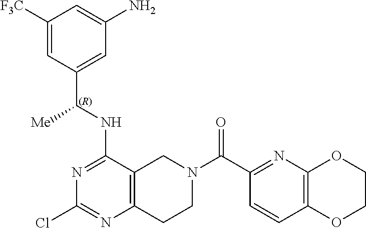 |
| Example 250. | 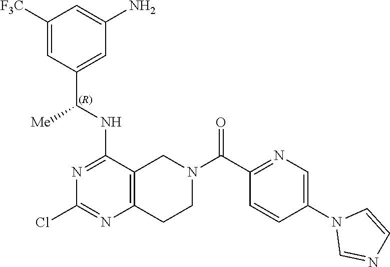 |
| Example 251. | 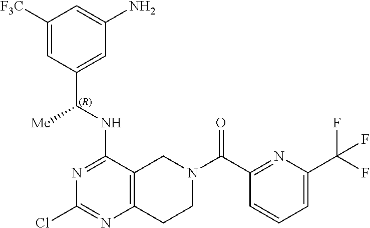 |
| Example 252. | 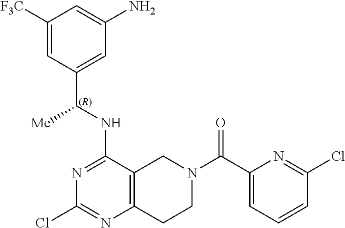 |
| Example 253. | 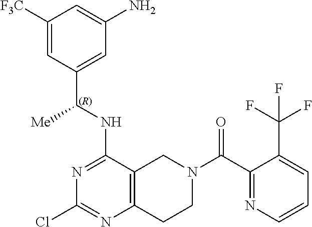 |
| Example 254. | 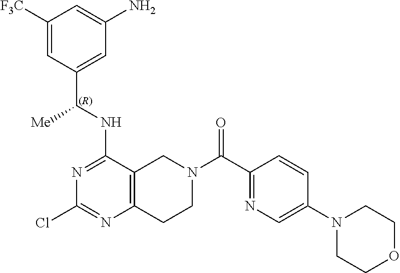 |
| Example 255. | 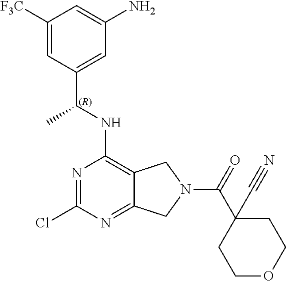 |
| Example 256. | 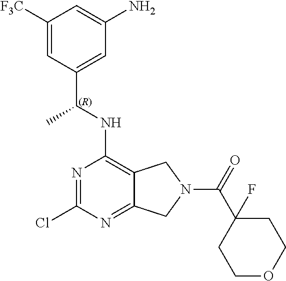 |
| Example 257. | 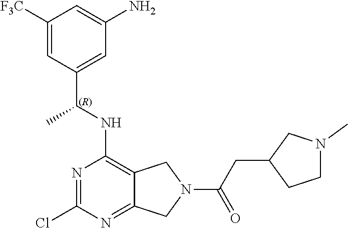 |
| Example 258. | 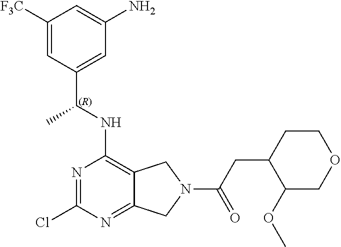 |
| Example 259. | 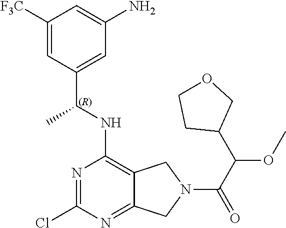 |
| Example 260. | 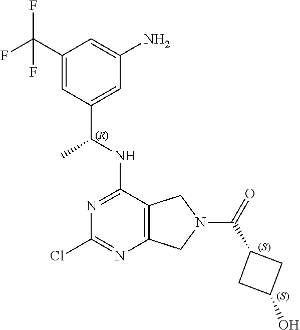 |
| Example 261. | 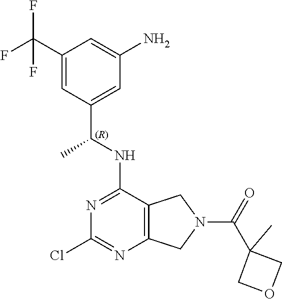 |
| Example 262. | 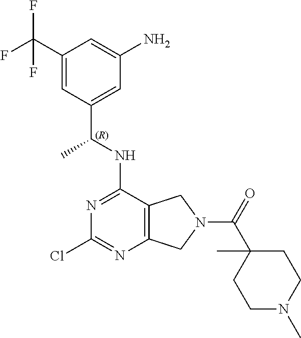 |
| Example 263. | 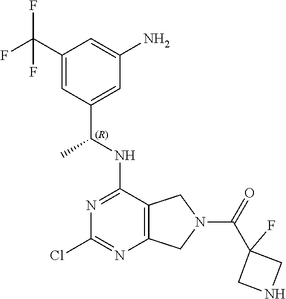 |
| Example 264. | 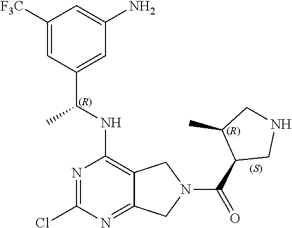 |
| Example 265. | 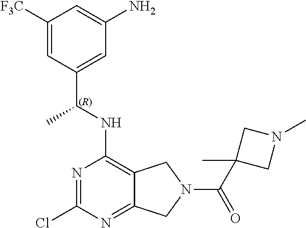 |
| Example 266. | 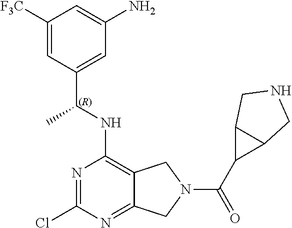 |
| Example 267. | 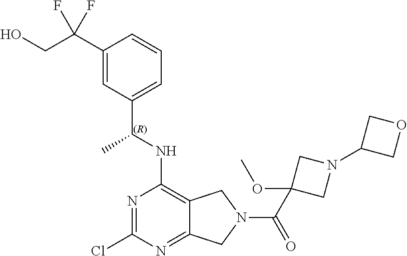 |
| Example 268. | 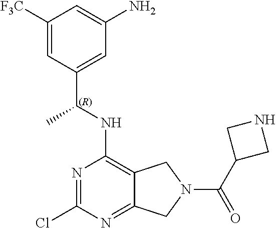 |
| Example 269. | 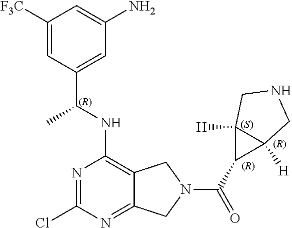 |
| Example 270. | 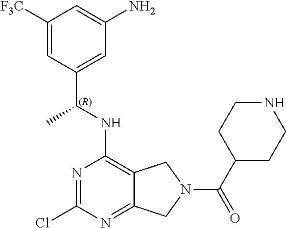 |
| Example 271. | 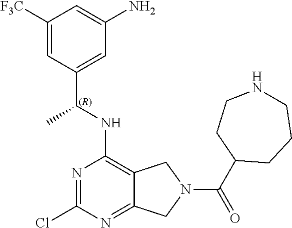 |
| Example 272. | 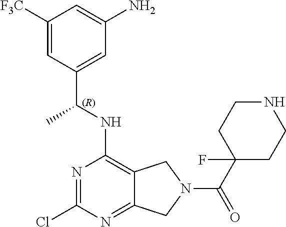 |
| Example 273. | 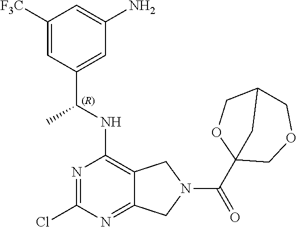 |
| Example 274. | 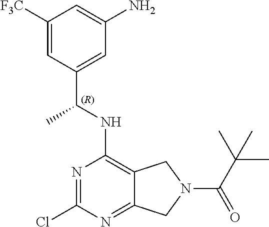 |
| Example 275. | 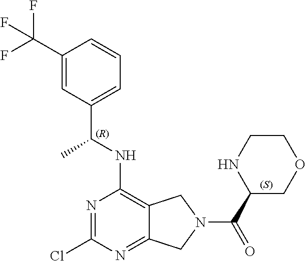 |
| Example 276. | 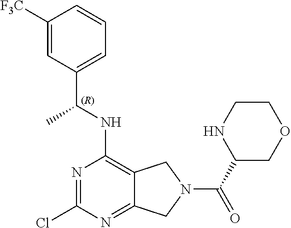 |
| Example 277. | 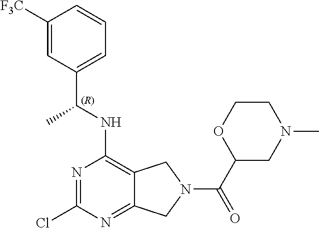 |
| Example 278. | 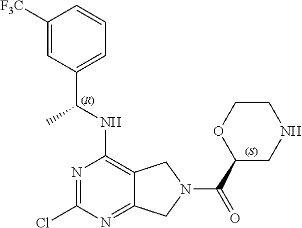 |
| Example 279. | 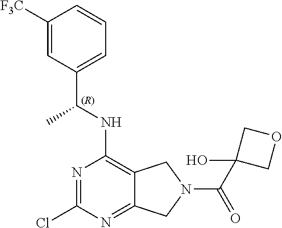 |
| Example 280. | 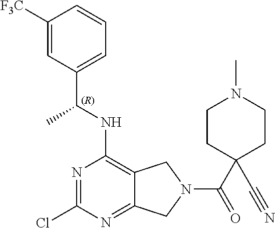 |
| Example 281. | 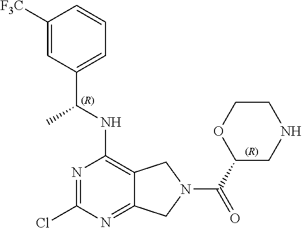 |
| Example 282. | 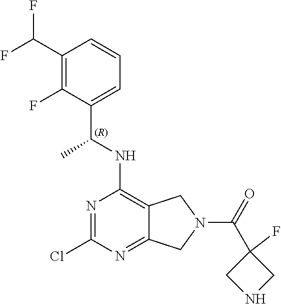 |
| Example 283. | 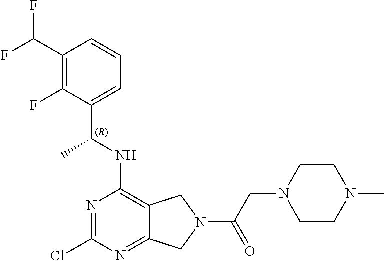 |
| Example 284. | 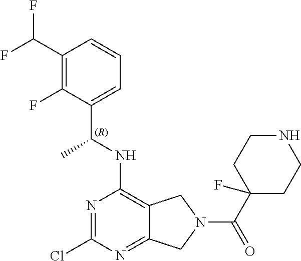 |
| Example 285. | 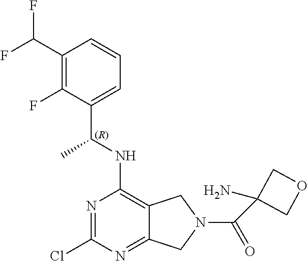 |
| Example 286. | 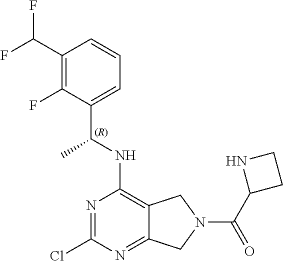 |
| Example 287. | 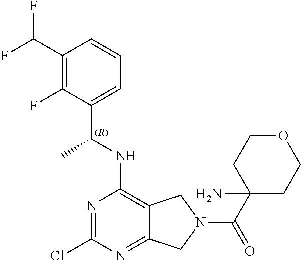 |
| Example 288. | 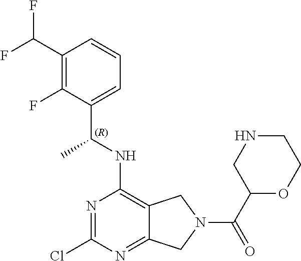 |
| Example 289. | 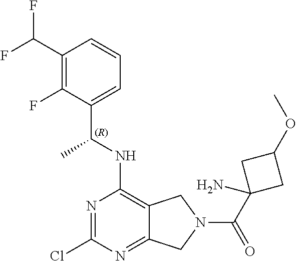 |
| Example 290. | 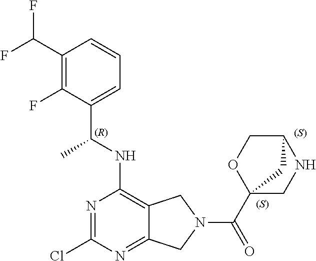 |
| Example 291. | 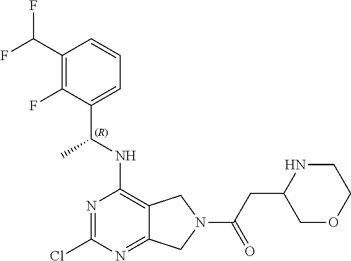 |
| Example 292. | 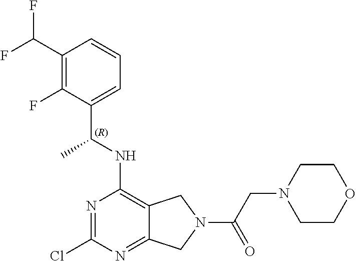 |
| Example 293. | 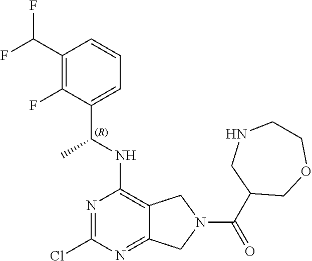 |
| Example 294. | 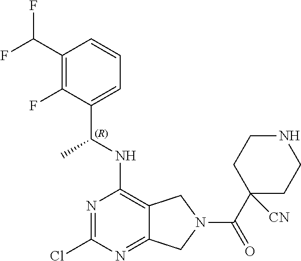 |
| Example 295. | 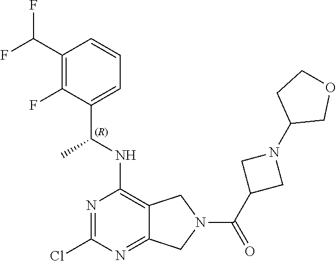 |
| Example 296. | 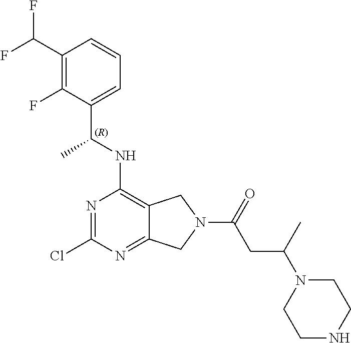 |
| Example 297. | 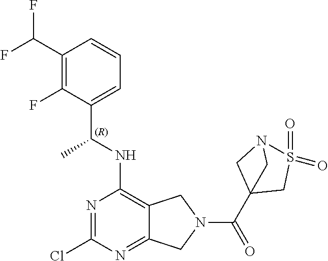 |
| Example 298. | 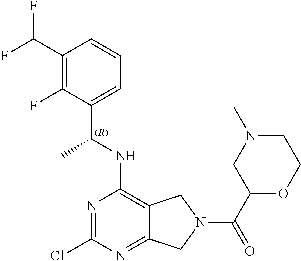 |
| Example 299. | 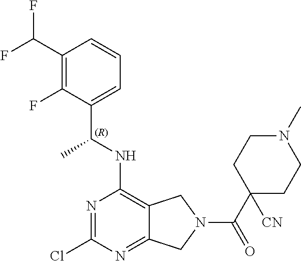 |
| Example 300. | 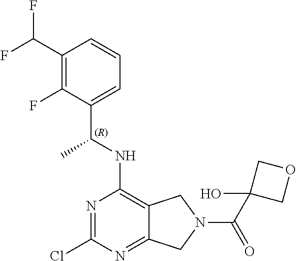 |
| Example 301. | 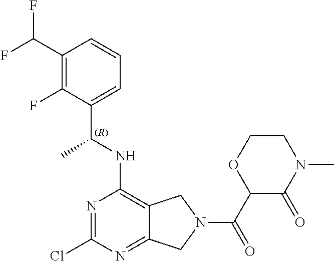 |
| Example 302. | 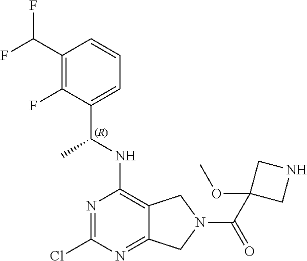 |
| Example 303. | 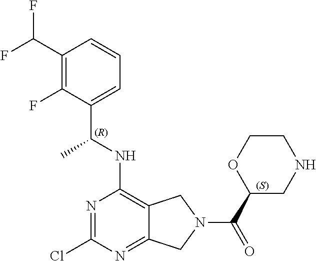 |
| Example 304. | 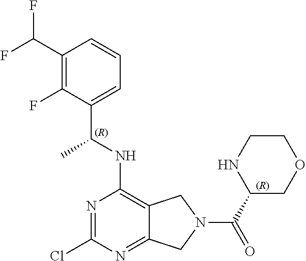 |
| Example 305. | 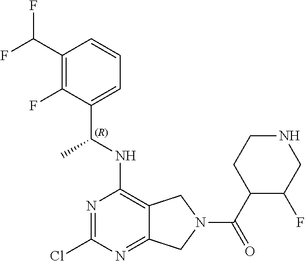 |
| Example 306. | 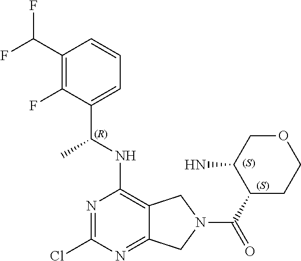 |
| Example 307. | 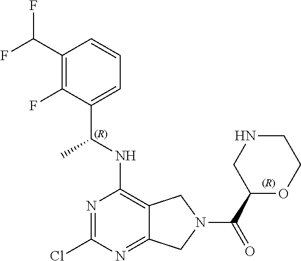 |
| Example 308. | 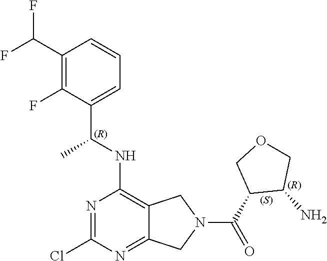 |
| Example 309. | 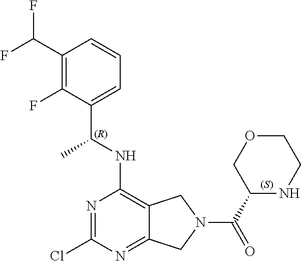 |
| Example 310. | 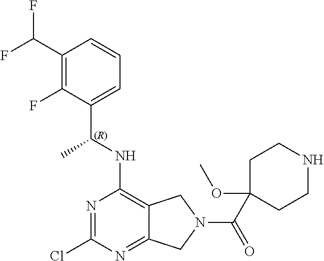 |
| Example 311. | 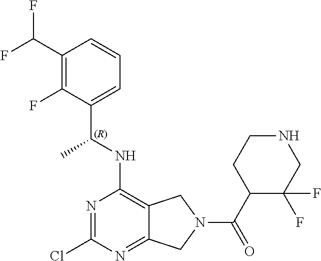 |
| Example 312. | 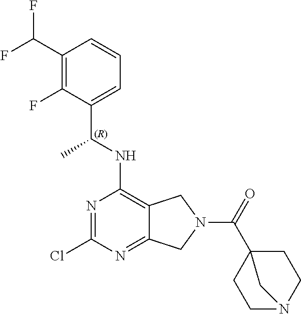 |
| Example 313. | 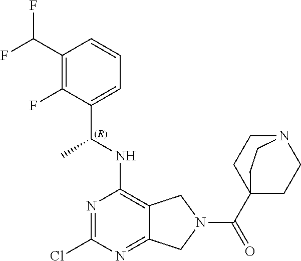 |
| Example 314. | 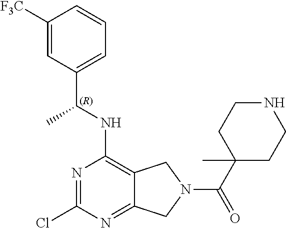 |
| Example 315. | 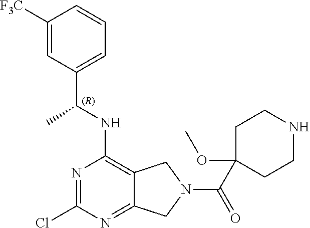 |
| Example 316. | 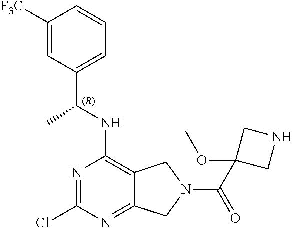 |
| Example 317. | 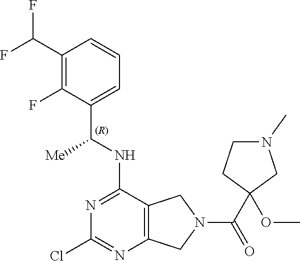 |
| Example 318. | 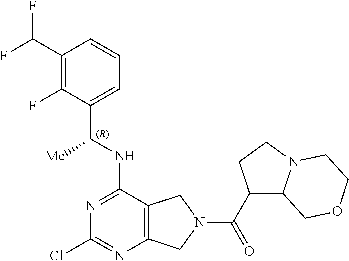 |
| Example 319. | 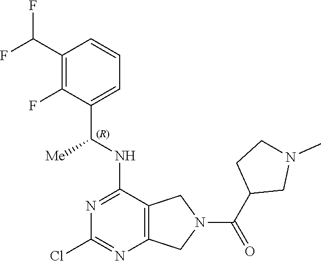 |
| Example 320. | 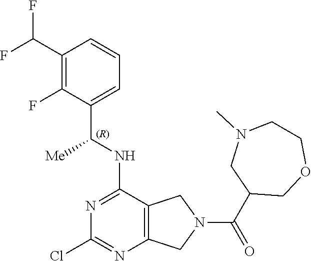 |
| Example 321. | 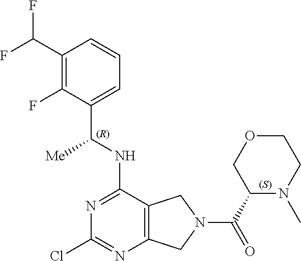 |
| Example 322. | 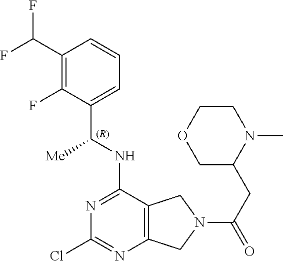 |
| Example 323. | 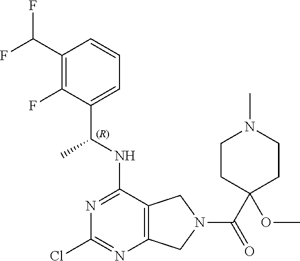 |
| Example 324. | 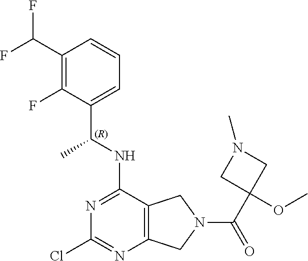 |
| Example 325. | 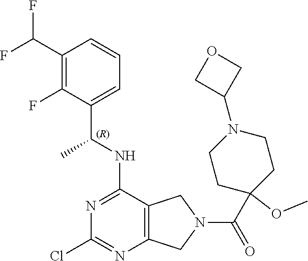 |
| Example 326. | 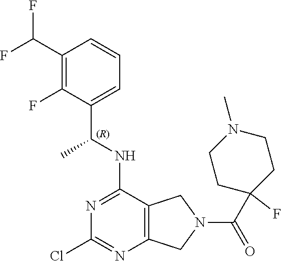 |
| Example 327. | 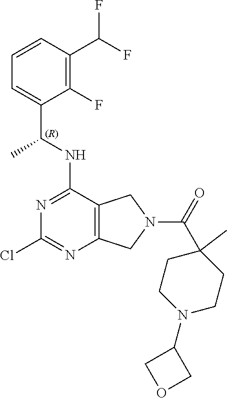 |
| Example 328. | 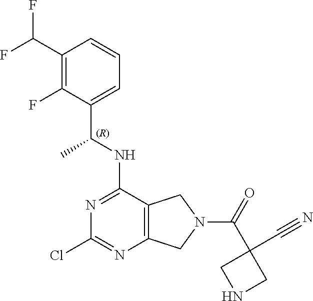 |
| Example 329. | 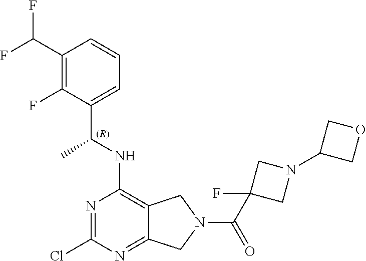 |
| Example 330. | 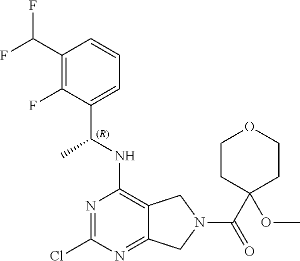 |
| Example 331. | 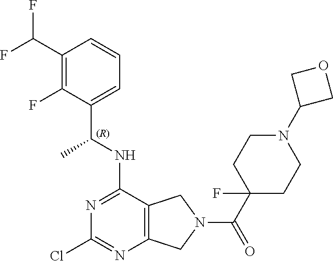 |
| Example 332. | 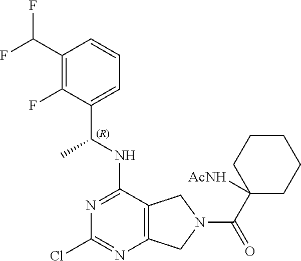 |
| Example 333. | 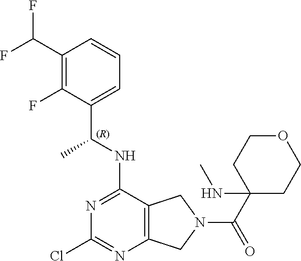 |
| Example 334. | 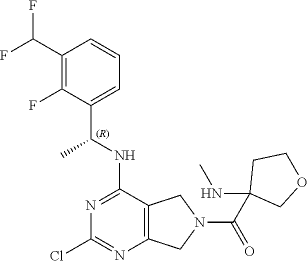 |
| Example 335. | 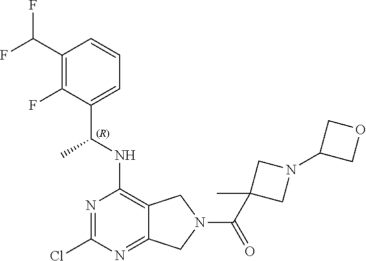 |
| Example 336. | 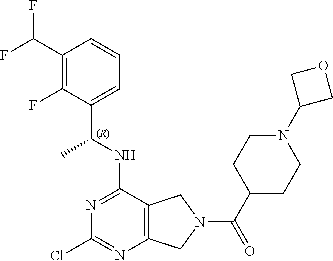 |
| Example 337. | 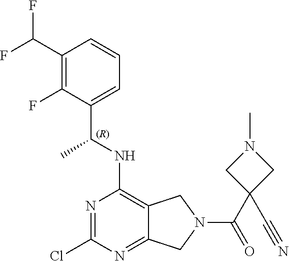 |
| Example 338. | 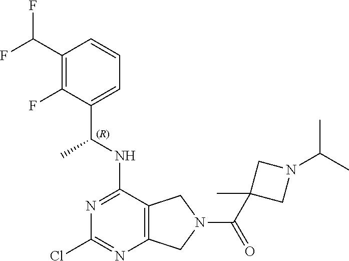 |
| Example 339. | 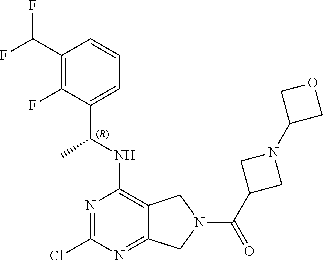 |
| Example 340. | 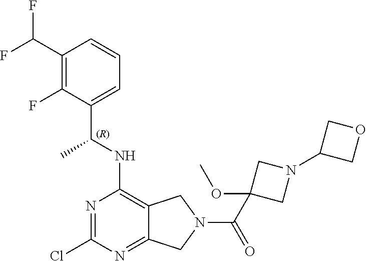 |
| Example 341. | 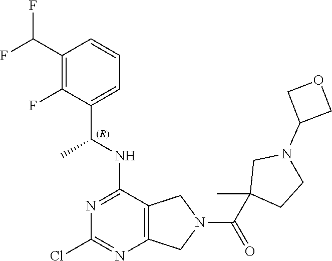 |
| Example 342. | 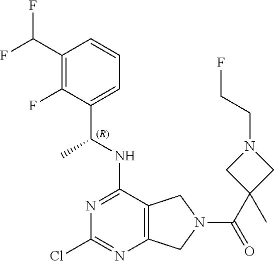 |
| Example 343. | 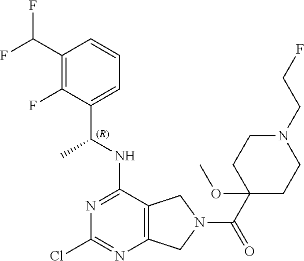 |
| Example 344. | 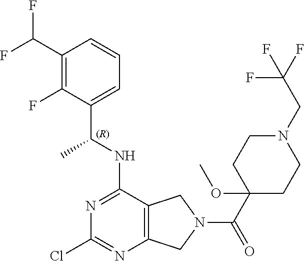 |
| Example 345. | 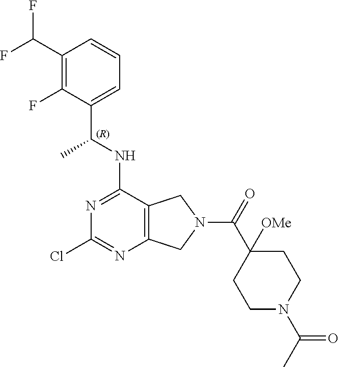 |
| Example 346. | 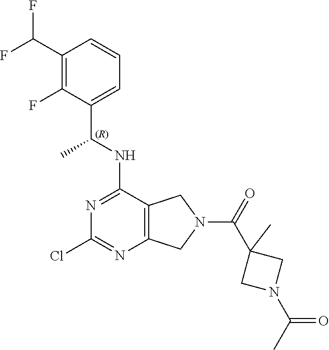 |
| Example 347. | 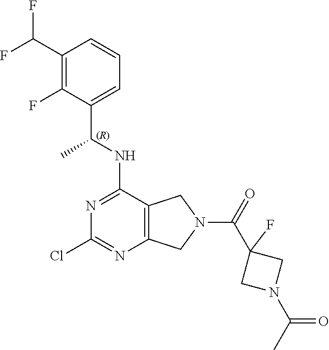 |
| Example 348. | 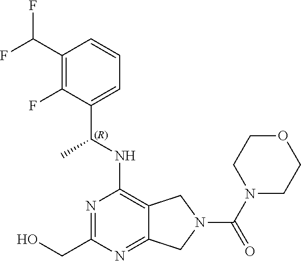 |
| Example 349. | 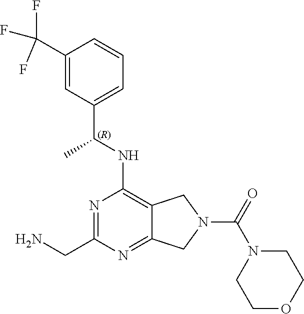 |
| Example 350. | 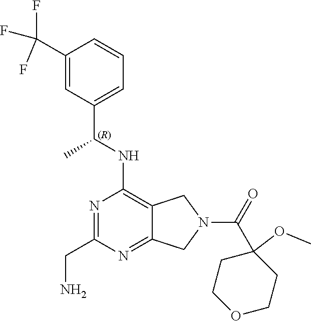 |
| Example 351. | 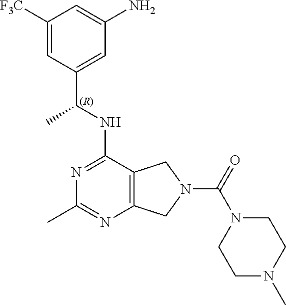 |
| Example 352. | 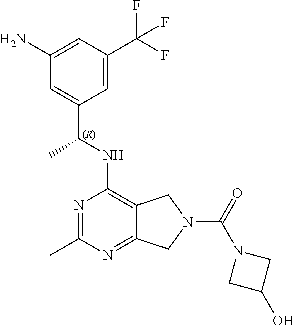 |
| Example 353. | 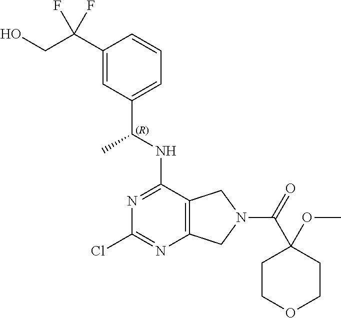 |
| Example 354. | 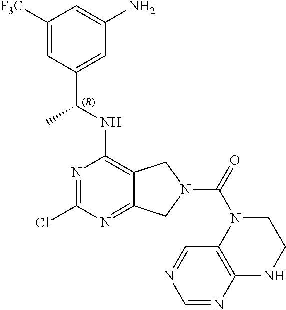 |
| Example 355. | 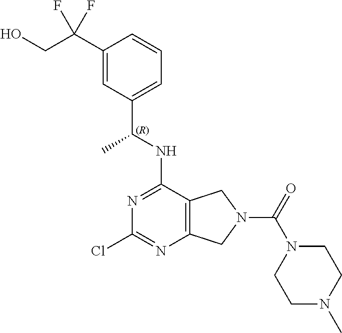 |
| Example 356. | 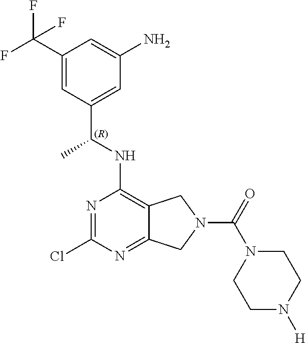 |
| Example 357. | 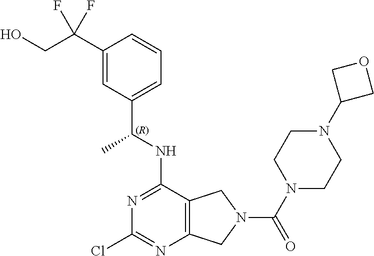 |
| Example 358. | 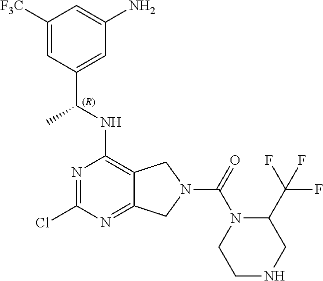 |
| Example 359. | 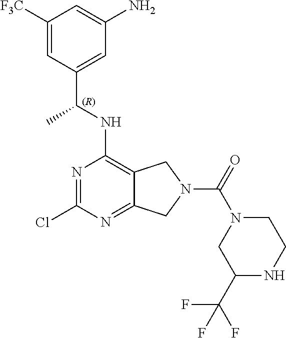 |
| Example 360. | 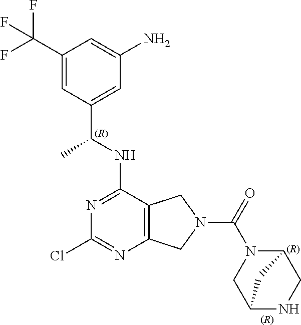 |
| Example 361. | 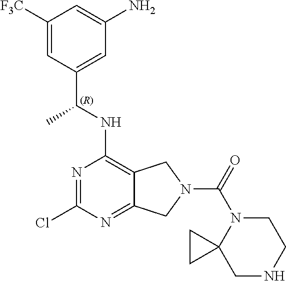 |
| Example 362. | 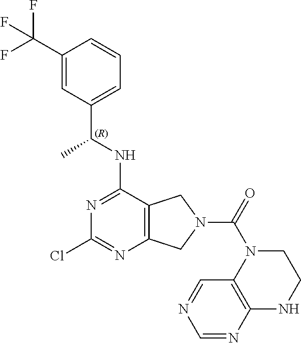 |
| Example 363. | 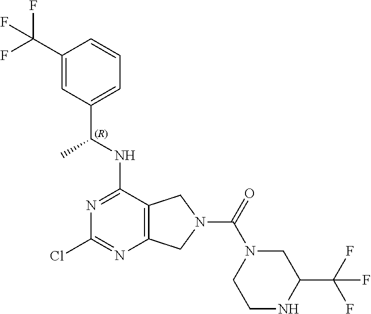 |
| Example 364. | 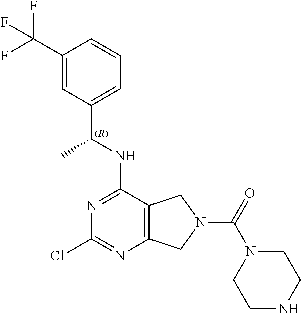 |
| Example 365. | 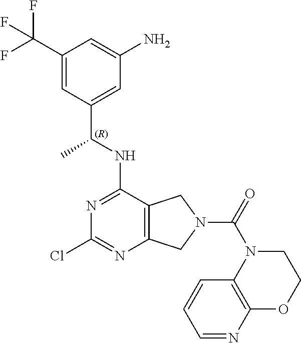 |
| Example 366. | 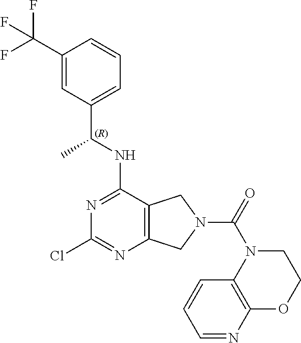 |
| Example 367. | 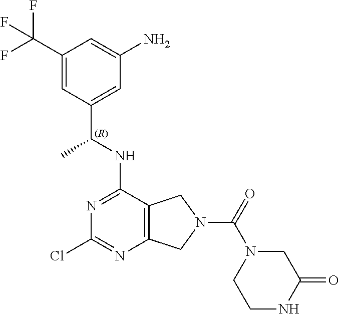 |
| Example 368. | 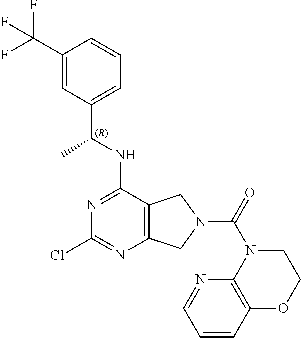 |
| Example 369. | 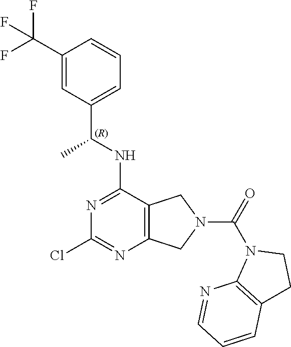 |
| Example 370. | 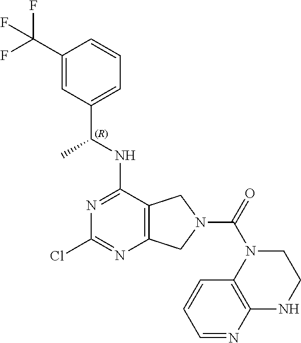 |
| Example 371. | 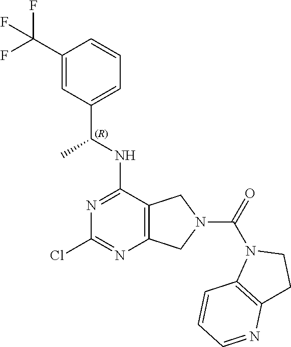 |
| Example 372. | 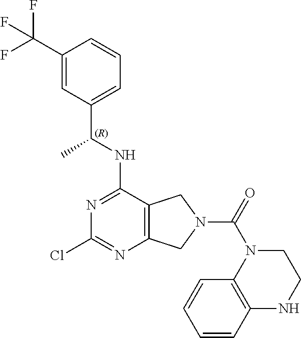 |
| Example 373. | 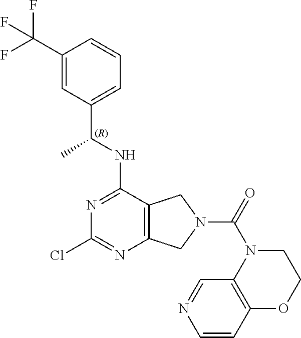 |
| Example 374. | 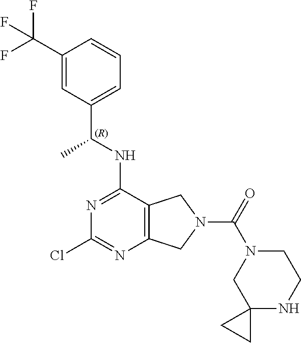 |
| Example 375. | 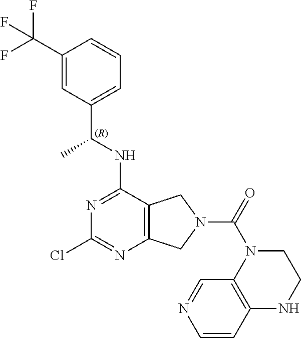 |
| Example 376. | 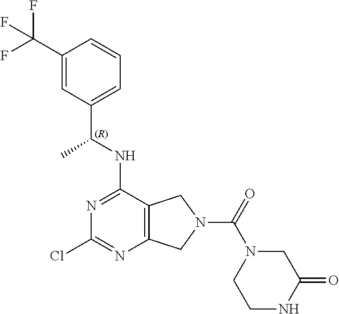 |
| Example 377. | 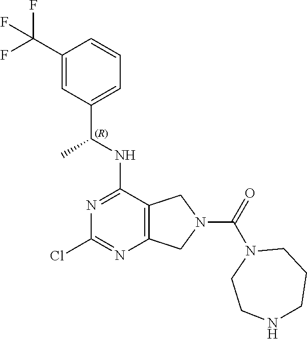 |
| Example 378. | 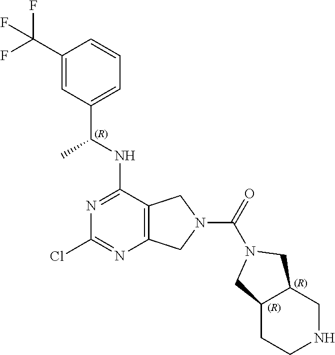 |
| Example 379. | 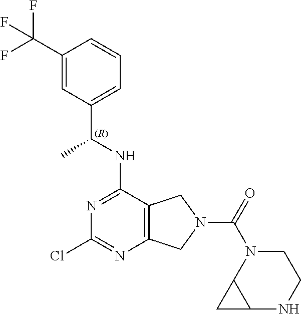 |
| Example 380. | 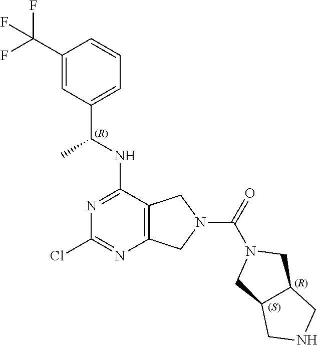 |
| Example 381. | 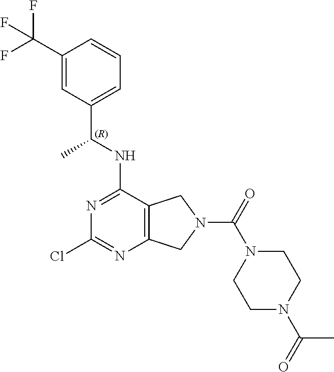 |
| Example 382. | 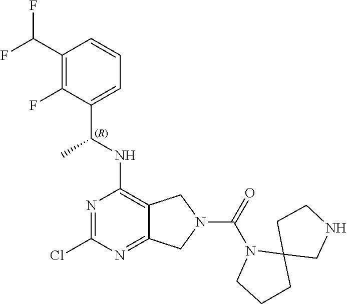 |
| Example 383. | 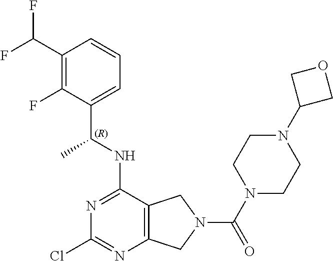 |
| Example 384. | 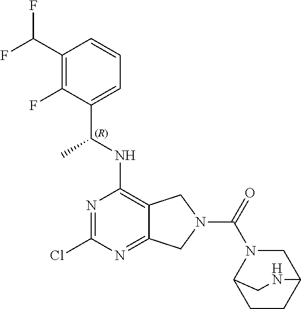 |
| Example 385. | 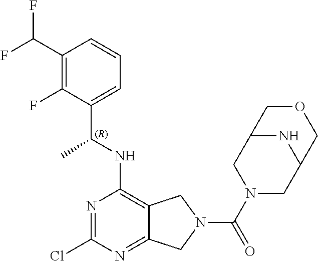 |
| Example 386. | 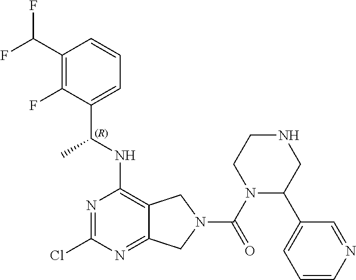 |
| Example 387. | 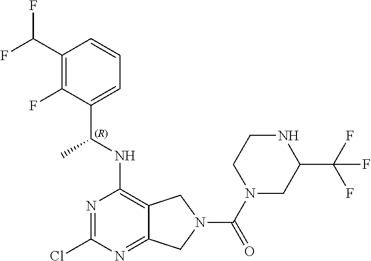 |
| Example 388. | 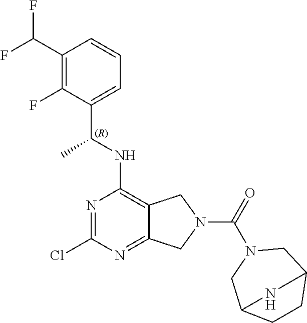 |
| Example 389. | 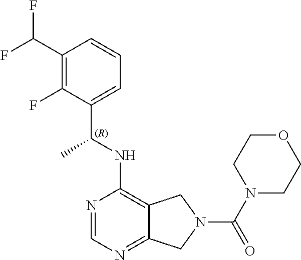 |
| Example 390. | 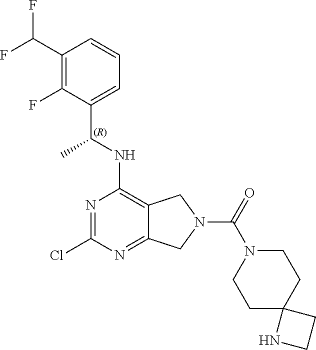 |
| Example 391. | 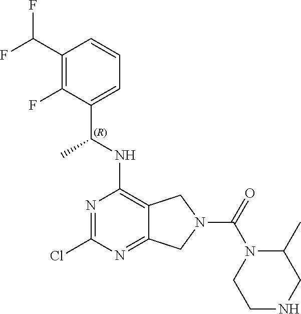 |
| Example 392. | 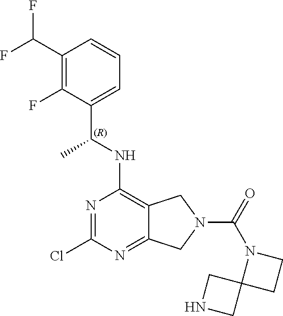 |
| Example 393. | 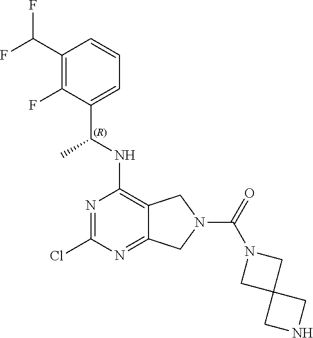 |
| Example 394. | 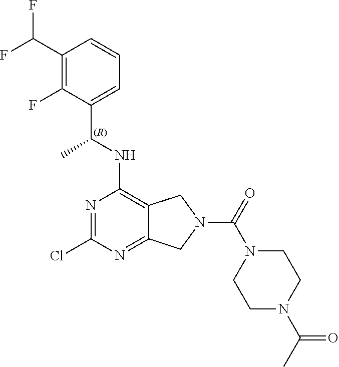 |
| Example 395. | 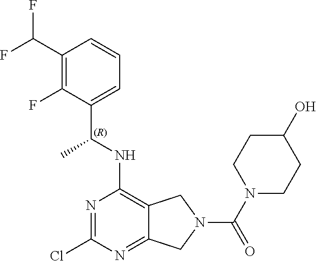 |
| Example 396. | 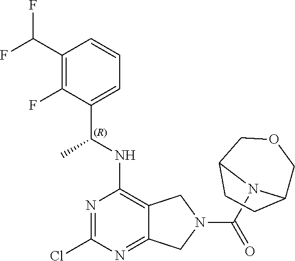 |
| Example 397. | 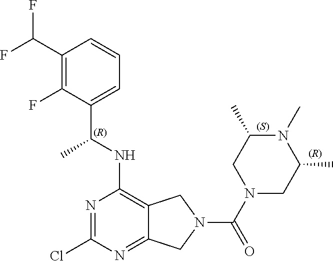 |
| Example 398. | 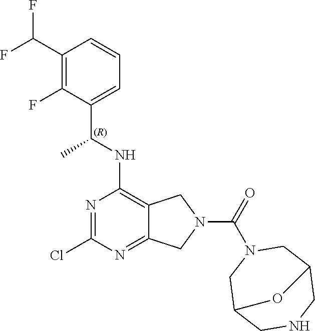 |
| Example 399. | 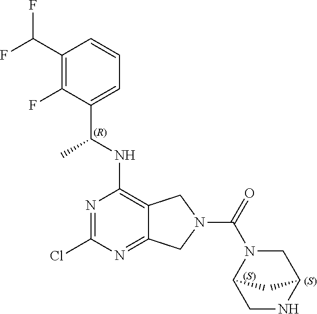 |
| Example 400. | 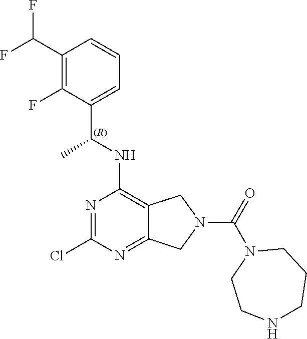 |
| Example 401. | 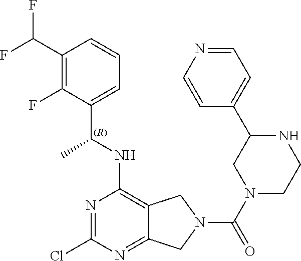 |
| Example 402. | 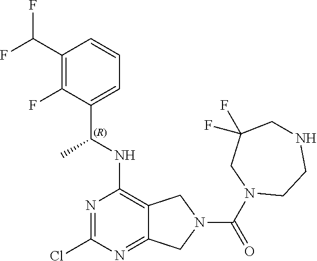 |
| Example 403. | 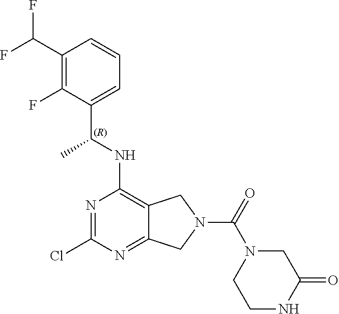 |
| Example 404. | 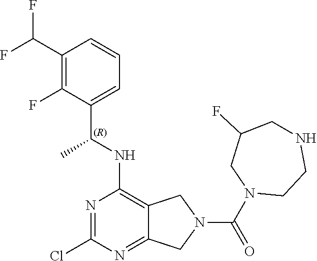 |
| Example 405. | 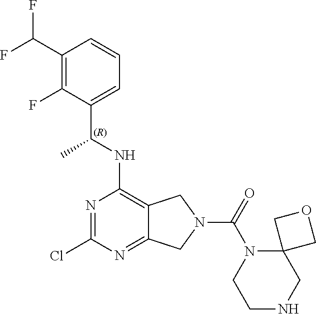 |
| Example 406. | 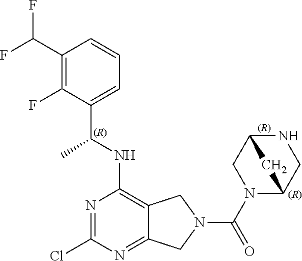 |
| Example 407. | 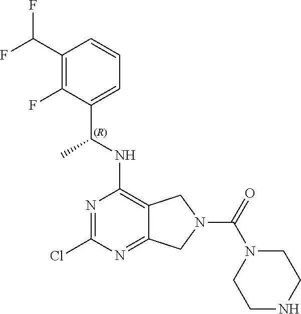 |
| Example 408. | 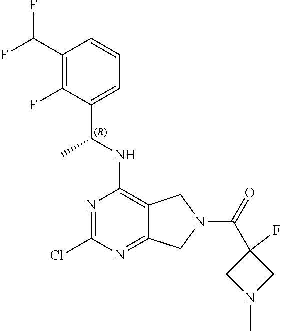 |
| Example 409. | 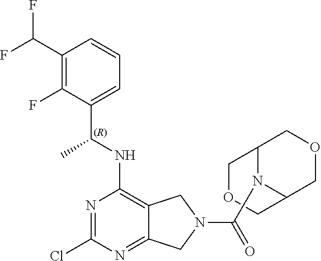 |
| Example 410. | 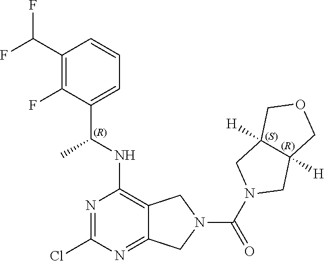 |
| Example 411. | 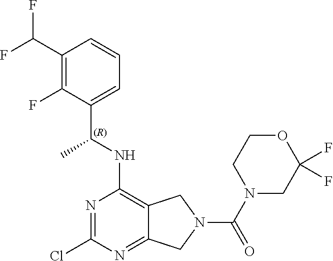 |
| Example 412. | 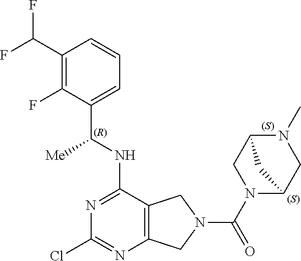 |
| Example 413. | 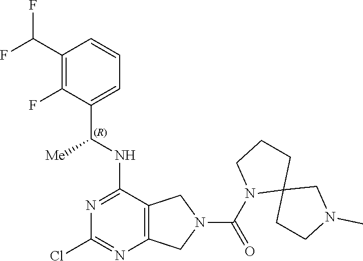 |
| Example 414. | 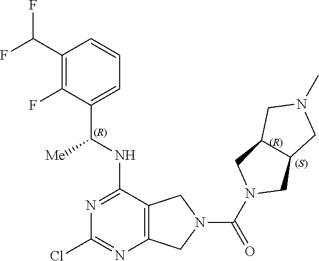 |
| Example 415. | 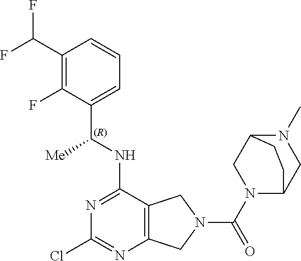 |
| Example 416. | 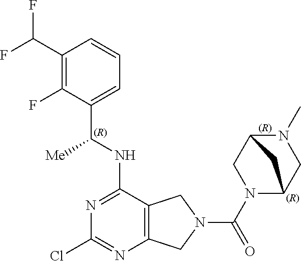 |
| Example 417. | 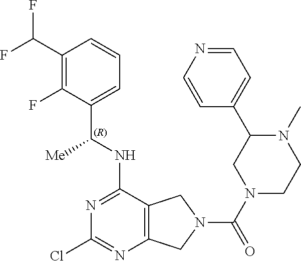 |
| Example 418. | 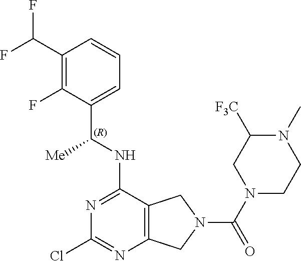 |
| Example 419. | 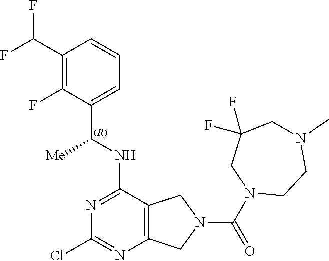 |
| Example 420. | 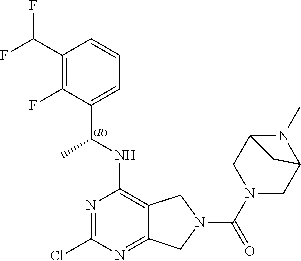 |
| Example 421. | 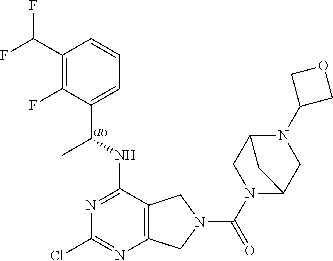 |
| Example 422. | 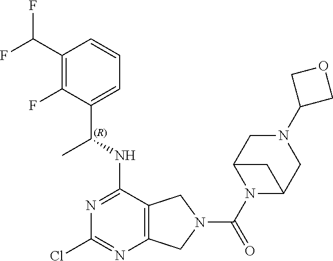 |
| Example 423. | 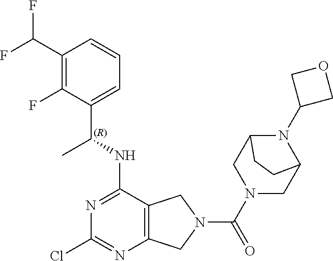 |
| Example 424. | 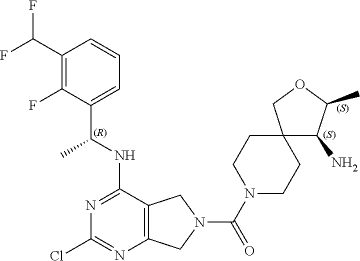 |
| Example 425. | 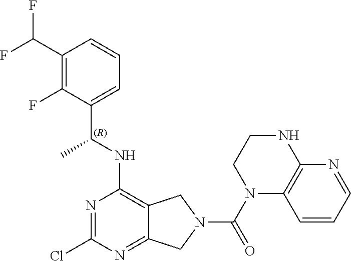 |
| Example 426. | 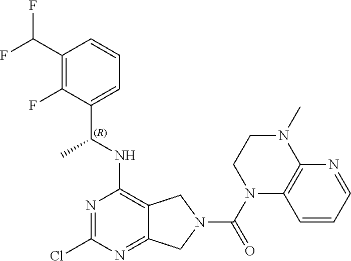 |
| Example 427. | 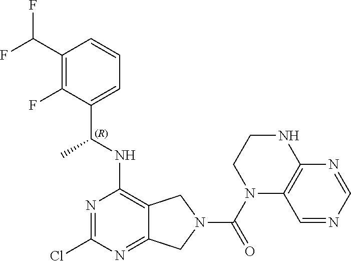 |
| Example 428. | 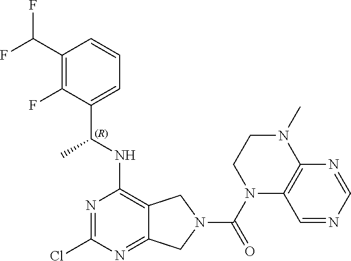 |
| Example 429. | 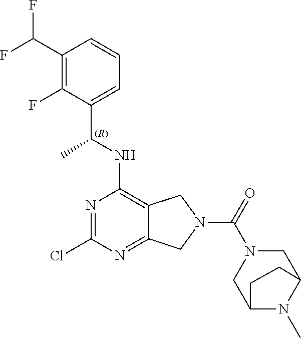 |
| Example 430. | 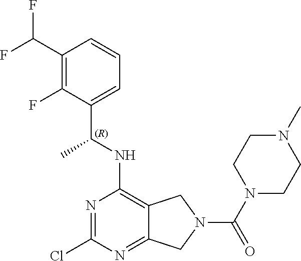 |
| Example 431. | 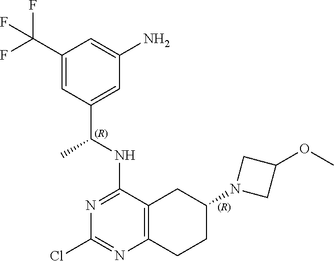 |
| Example 432. | 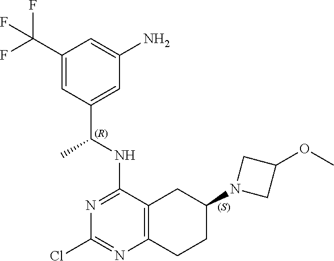 |
| Example 433. | 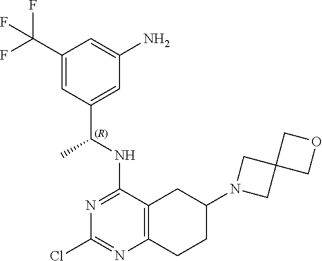 |
| Example 434. | 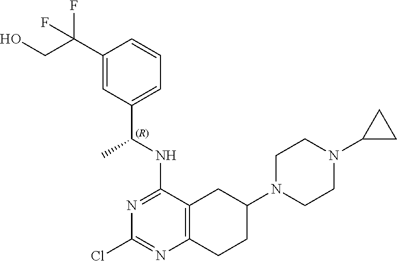 |
| Example 435. | 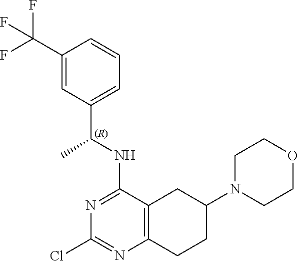 |
| Example 436. | 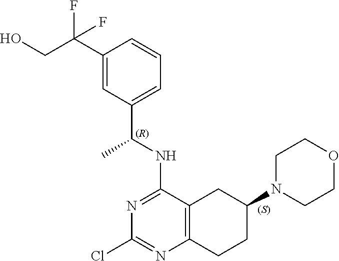 |
| Example 437. | 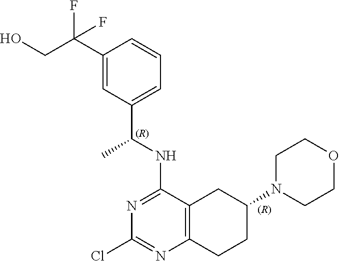 |
| Example 438. | 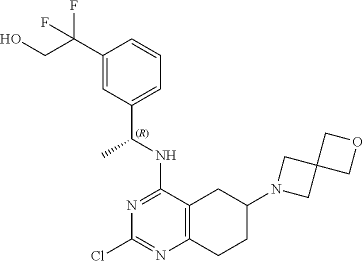 |
| Example 439. | 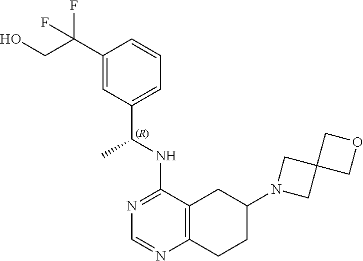 |
| Example 440. | 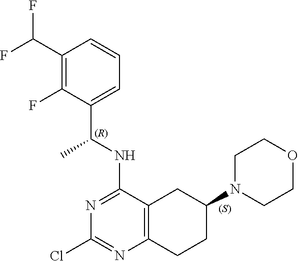 |
| Example 441. | 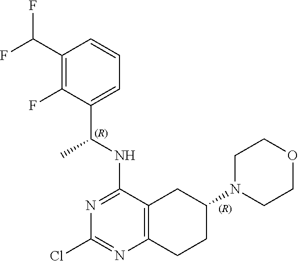 |
| Example 442. | 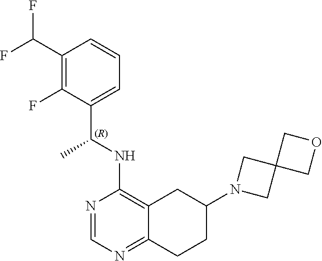 |
| Example 443. | 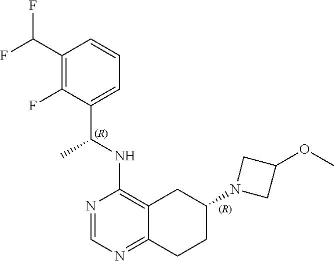 |
| Example 444. | 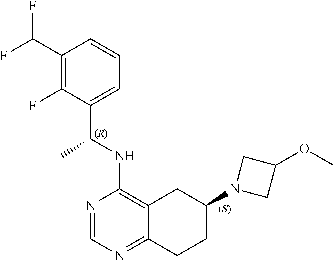 |
| Example 445. | 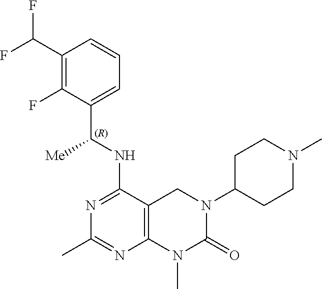 |
| Example 446. | 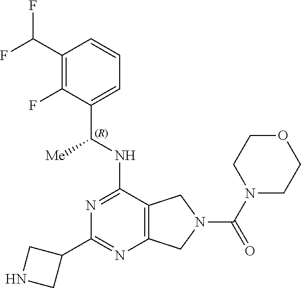 |
| Example 447. | 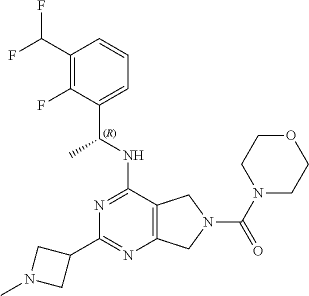 |
| Example 448. | 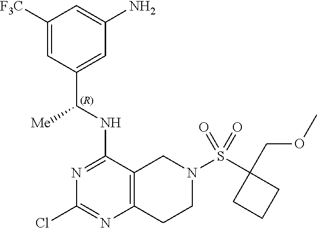 |
| Example 449. | 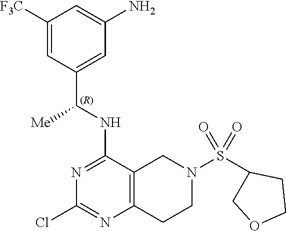 |
| Example 450. | 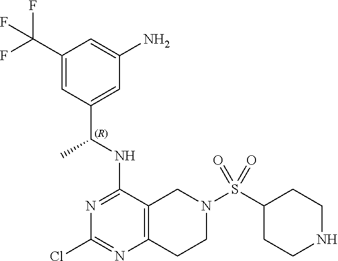 |
| Example 451. | 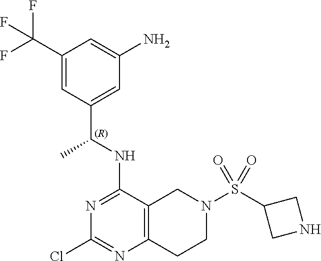 |
| Example 452. | 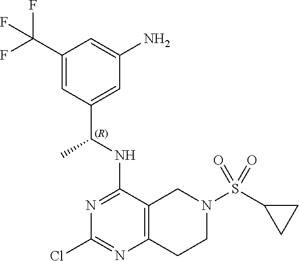 |
| Example 453. | 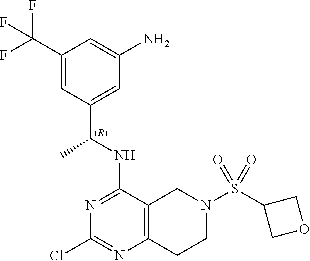 |
| Example 454. | 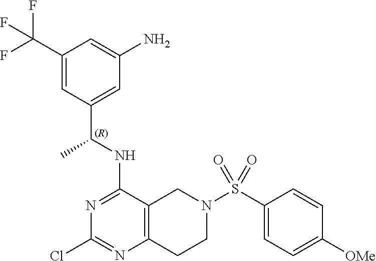 |
| Example 455. | 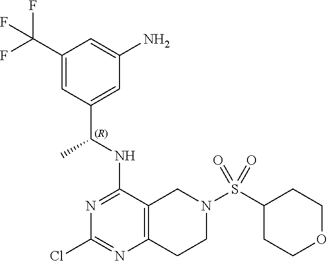 |
| Example 456. | 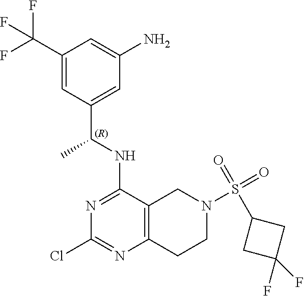 |
| Example 457. | 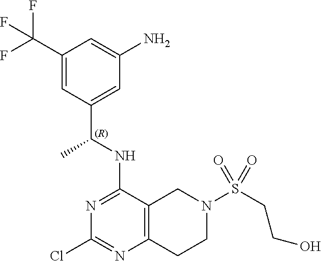 |
| Example 458. | 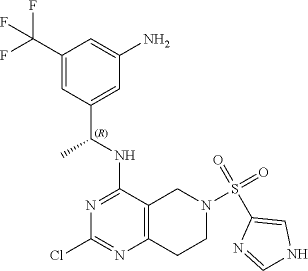 |
| Example 459. | 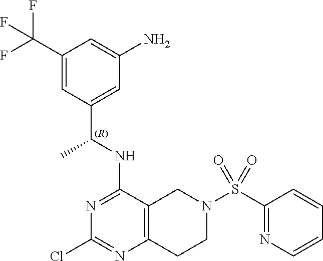 |
| Example 460. | 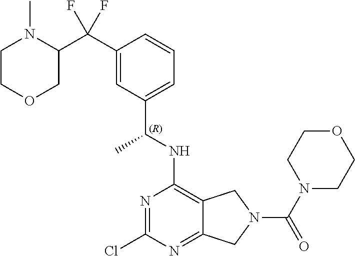 |
| Example 461. | 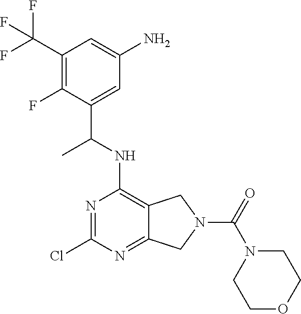 [SMILES: CC(Nc1nc(Cl)nc2c1CN(C(=O)N1CCOCC1)C2)c1cc(N)cc(C(F)(F)F)c1F] |
| Example 462. | 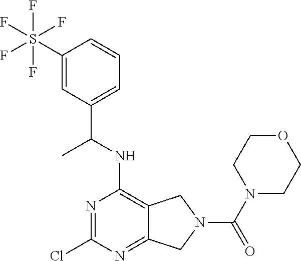 [SMILES: CC(Nc1nc(Cl)nc2c1CN(C(=O)N1CCOCC1)C2)c1cccc(S(F)(F)(F)(F)F)c1] |
| Example 463. | 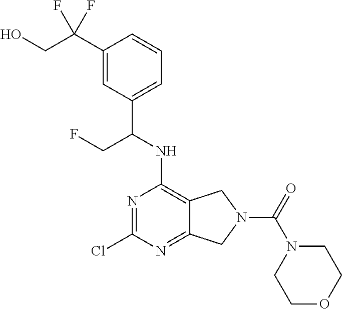 [SMILES: O=C(N1CCOCC1)N1Cc2nc(Cl)nc(NC(CF)c3cccc(C(F)(F)CO)c3)c2C1] |
| Example 464. | 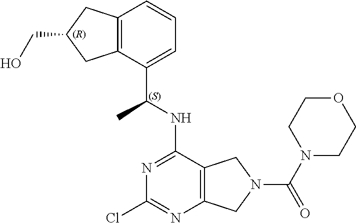 |
| Example 465. | 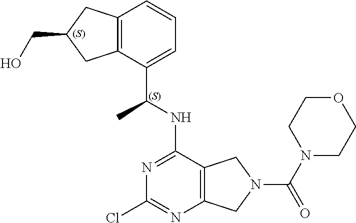 |
| Example 466. | 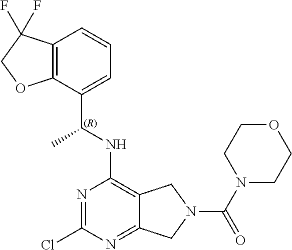 |
| Example 467. | 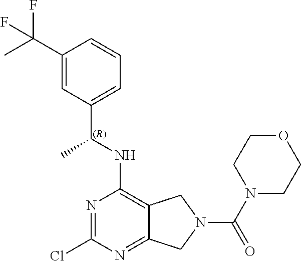 |
| Example 468. | 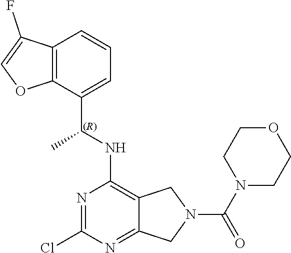 |
| Example 469. | 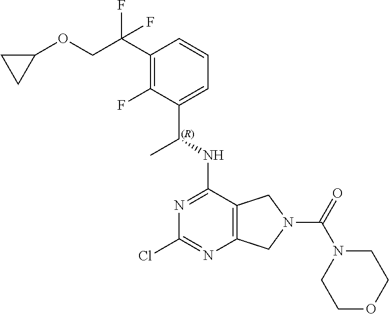 |
| Example 470. | 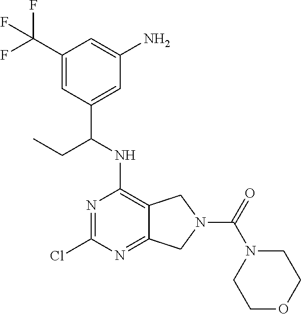 [SMILES: CCC(Nc1nc(Cl)nc2c1CN(C(=O)N1CCOCC1)C2)c1cc(N)cc(C(F)(F)F)c1] |
| Example 471. | 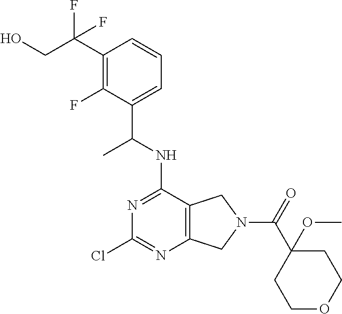 [SMILES: COC1(C(=O)N2Cc3nc(Cl)nc(NC(C)c4cccc(C(F)(F)CO)c4F)c3C2)CCOCC1] |
| Example 472. | 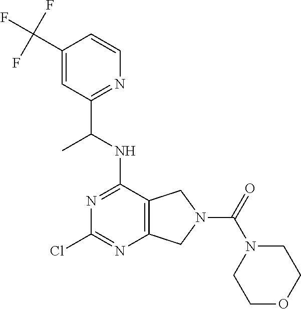 [SMILES: CC(Nc1nc(Cl)nc2c1CN(C(=O)N1CCOCC1)C2)c1cc(C(F)(F)F)ccn1] |
| Example 473. | 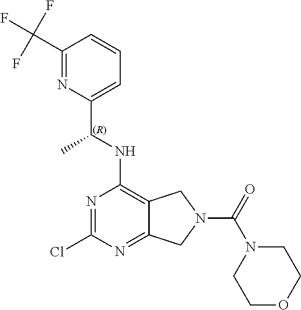 |
| Example 474. | 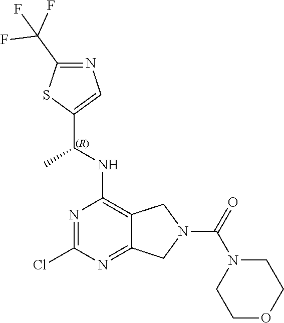 |
| Example 475. | 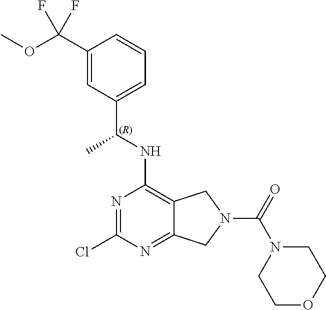 |
| Example 476. | 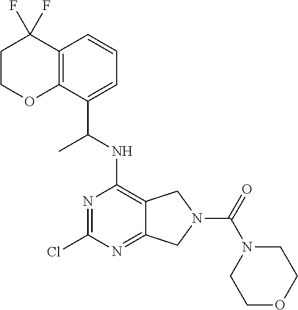 [SMILES: CC(Nc1nc(Cl)nc2c1CN(C(=O)N1CCOCC1)C2)c1cccc2c1OCCC2(F)F] |
| Example 477. | 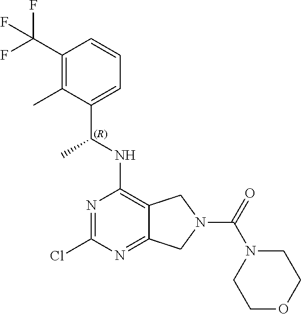 |
| Example 478. | 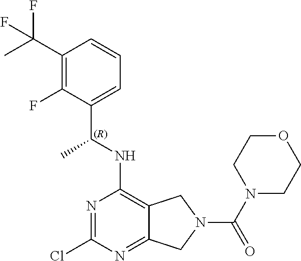 |
| Example 479. | 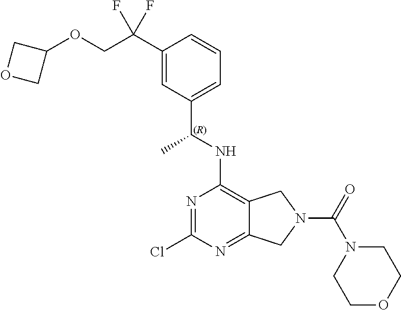 |
| Example 480. | 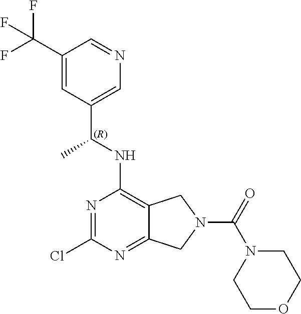 |
| Example 481. | 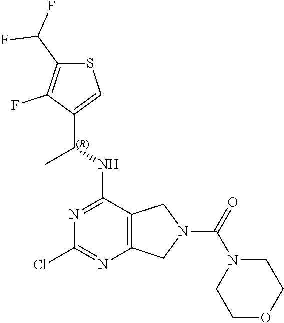 |
| Example 482. | 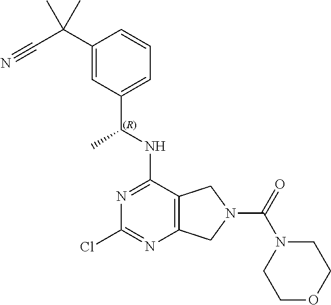 |
| Example 483. | 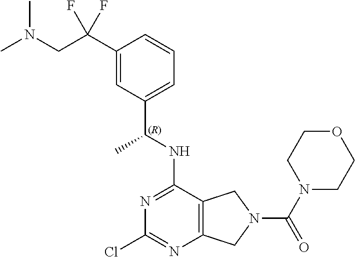 |
| Example 484. | 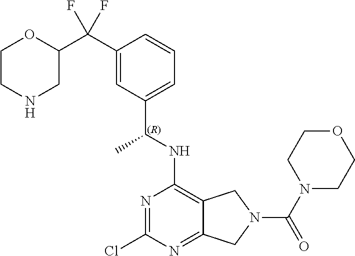 |
| Example 485. | 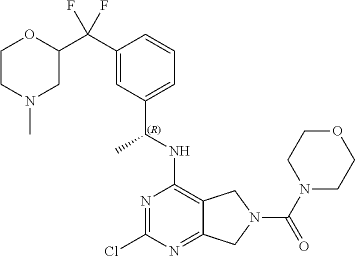 |
| Example 486. | 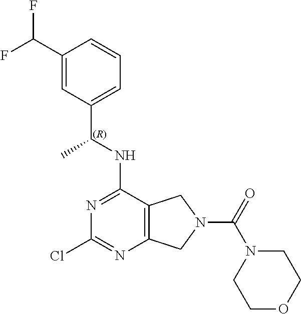 |
| Example 487. | 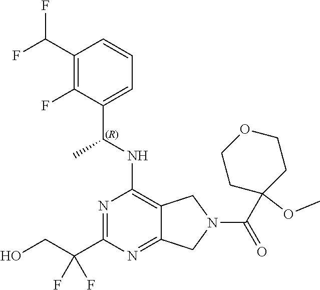 |
| Example 488. | 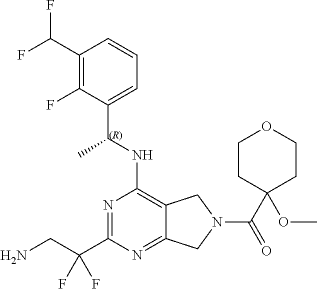 |
| Example 489. | 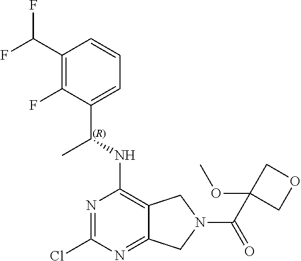 |
| Example 490. | 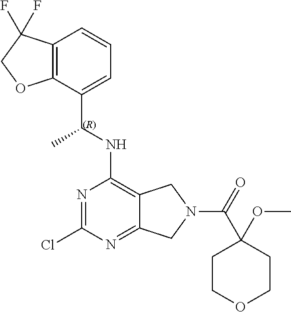 |
| Example 491. | 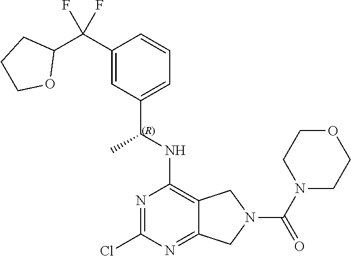 |
| Example 492. | 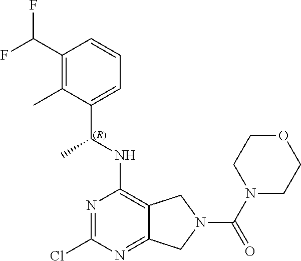 |
| Example 493. | 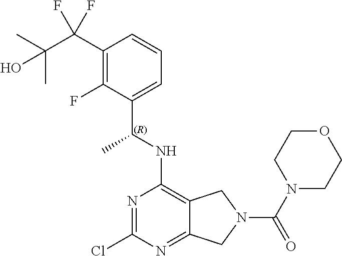 |
| Example 494. | 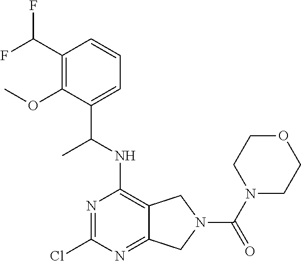 [SMILES: COc1c(C(F)F)cccc1C(C)Nc1nc(Cl)nc2c1CN(C(=O)N1CCOCC1)C2] |
| Example 495. | 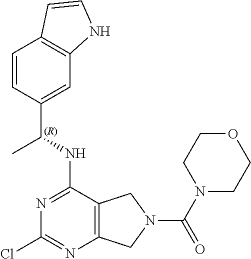 |
| Example 496. | 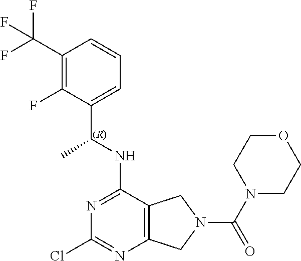 |
| Example 497. | 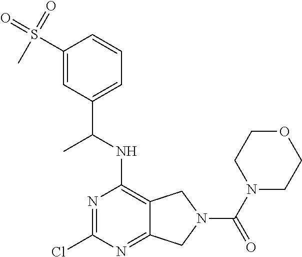 [SMILES: CC(Nc1nc(Cl)nc2c1CN(C(=O)N1CCOCC1)C2)c1cccc(S(C)(=O)=O)c1] |
| Example 498. | 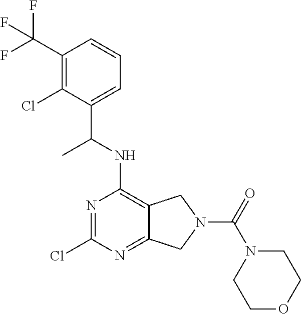 [SMILES: CC(Nc1nc(Cl)nc2c1CN(C(=O)N1CCOCC1)C2)c1cccc(C(F)(F)F)c1Cl] |
| Example 499. | 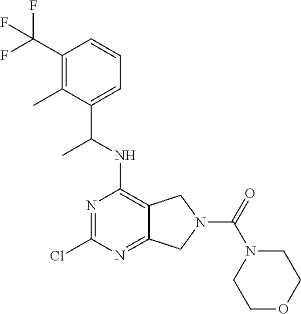 [SMILES: Cc1c(C(C)Nc2nc(Cl)nc3c2CN(C(=O)N2CCOCC2)C3)cccc1C(F)(F)F] |
| Example 500. | 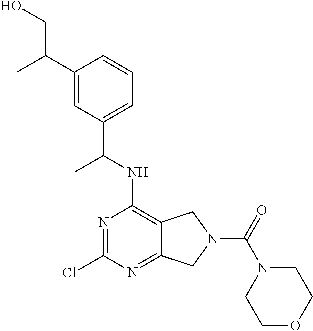 [SMILES: CC(CO)c1cccc(C(C)Nc2nc(Cl)nc3c2CN(C(=O)N2CCOCC2)C3)c1] |
| Example 501. | 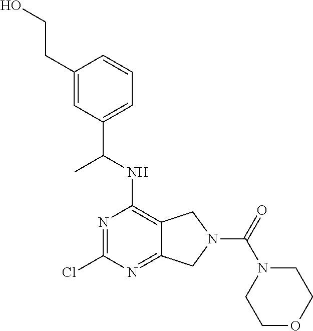 [SMILES: CC(Nc1nc(Cl)nc2c1CN(C(=O)N1CCOCC1)C2)c1cccc(CCO)c1] |
| Example 502. | 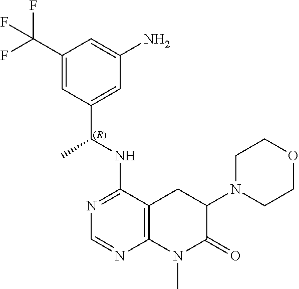 |
| Example 503. | 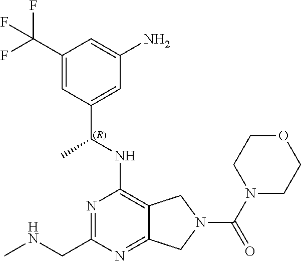 |
| Example 504. | 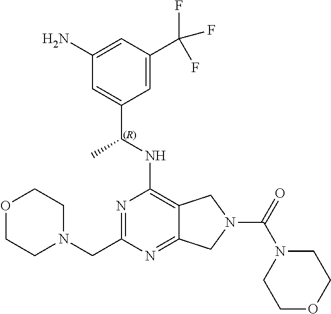 |
| Example 505. | 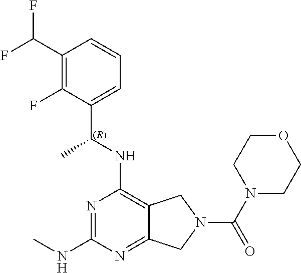 |
| Example 506. | 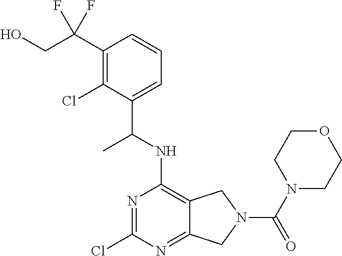 [SMILES: CC(Nc1nc(Cl)nc2c1CN(C(=O)N1CCOCC1)C2)c1cccc(C(F)(F)CO)c1Cl] |
| Example 507. | 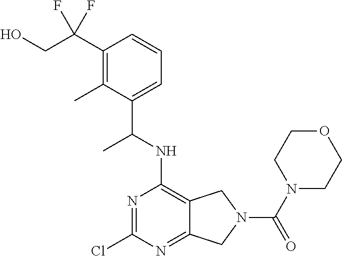 [SMILES: Cc1c(C(C)Nc2nc(Cl)nc3c2CN(C(=O)N2CCOCC2)C3)cccc1C(F)(F)CO] |
| Example 508. | 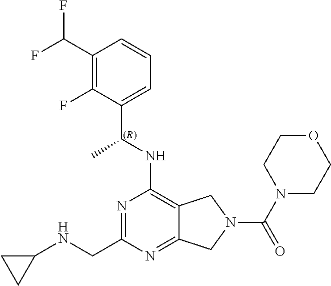 |
| Example 509. | 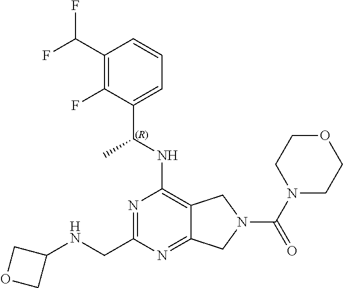 |
| Example 510. | 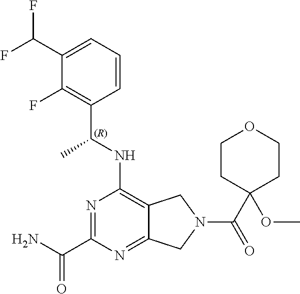 |
| Example 511. | 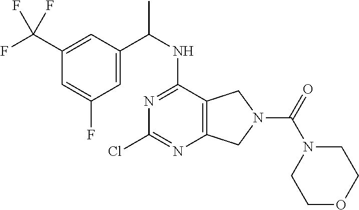 [SMILES: CC(Nc1nc(Cl)nc2c1CN(C(=O)N1CCOCC1)C2)c1cc(F)cc(C(F)(F)F)c1] |
| Example 512. | 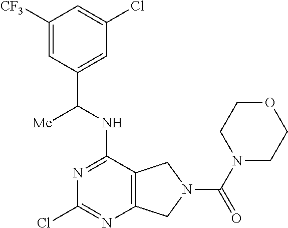 [SMILES: CC(Nc1nc(Cl)nc2c1CN(C(=O)N1CCOCC1)C2)c1cc(Cl)cc(C(F)(F)F)c1] |
| Example 513. | 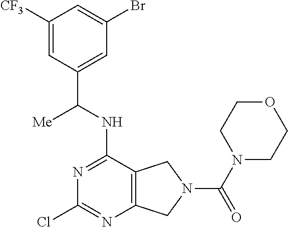 [SMILES: CC(Nc1nc(Cl)nc2c1CN(C(=O)N1CCOCC1)C2)c1cc(Br)cc(C(F)(F)F)c1] |
| Example 514. | 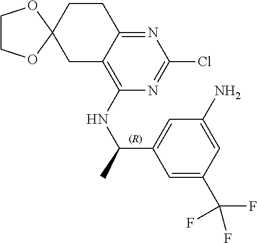 |
| Example 515. | 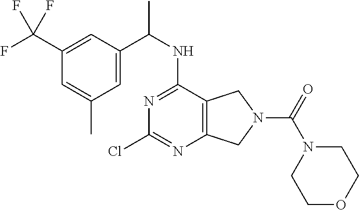 [SMILES: Cc1cc(C(C)Nc2nc(Cl)nc3c2CN(C(=O)N2CCOCC2)C3)cc(C(F)(F)F)c1] |
| Example 516. | 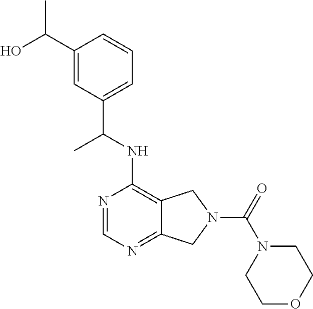 [SMILES: CC(O)c1cccc(C(C)Nc2ncnc3c2CN(C(=O)N2CCOCC2)C3)c1] |
| Example 517. | 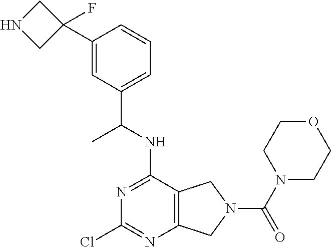 [SMILES: CC(Nc1nc(Cl)nc2c1CN(C(=O)N1CCOCC1)C2)c1cccc(C2(F)CNC2)c1] |
| Example 518. | 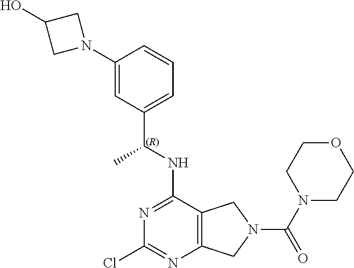 |
| Example 519. | 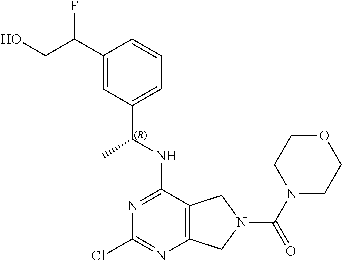 |
| Example 520. | 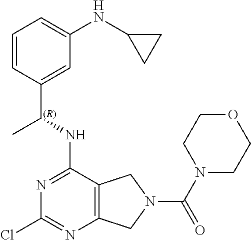 |
| Example 521. | 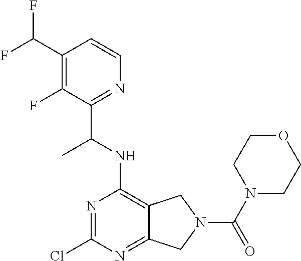 [SMILES: CC(Nc1nc(Cl)nc2c1CN(C(=O)N1CCOCC1)C2)c1nccc(C(F)F)c1F] |
| Example 522. | 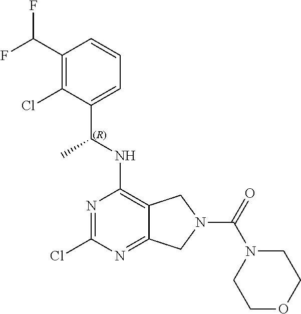 |
| Example 523. | 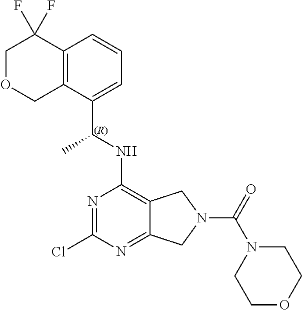 |
| Example 524. | 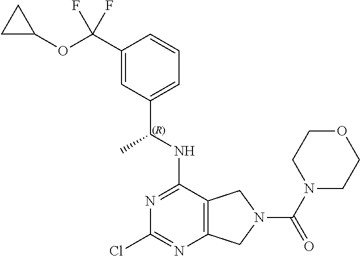 |
| Example 525. | 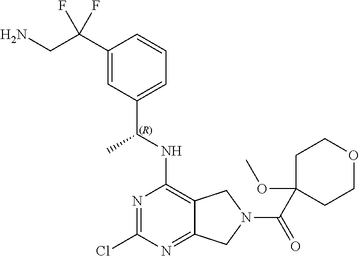 |
| Example 526. | 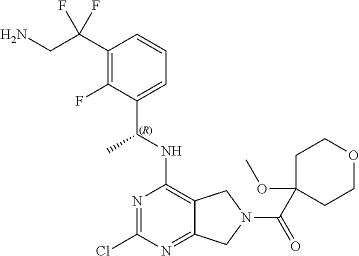 |
| Example 527. | 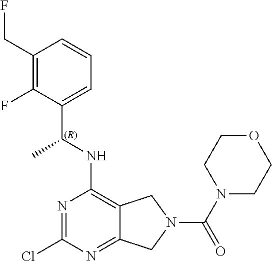 |
| Example 528. | 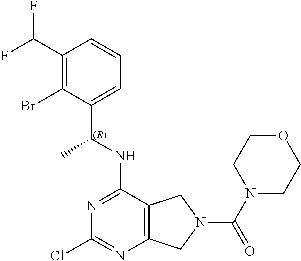 |
| Example 529. | 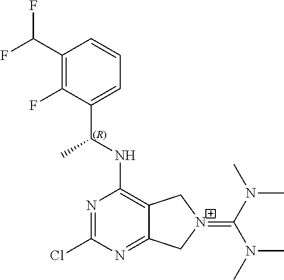 |
| Example 530. | 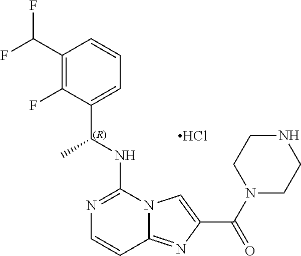 |
| Example 531. | 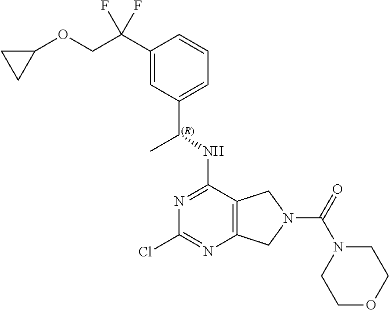 |
| Example 532. | 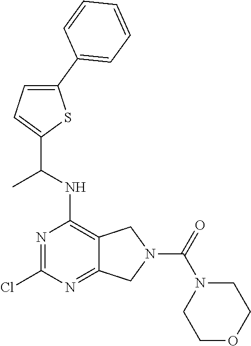 [SMILES: CC(Nc1nc(Cl)nc2c1CN(C(=O)N1CCOCC1)C2)c1ccc(-c2ccccc2)s1] |
| Example 533. | 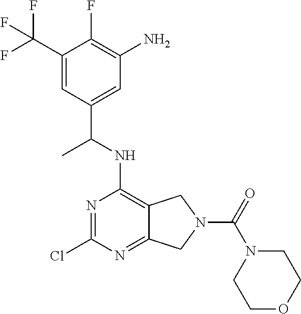 [SMILES: CC(Nc1nc(Cl)nc2c1CN(C(=O)N1CCOCC1)C2)c1cc(N)c(F)c(C(F)(F)F)c1] |
| Example 534. | 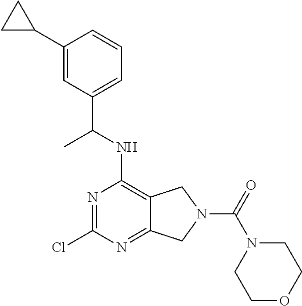 [SMILES: CC(Nc1nc(Cl)nc2c1CN(C(=O)N1CCOCC1)C2)c1cccc(C2CC2)c1] |
| Example 535. | 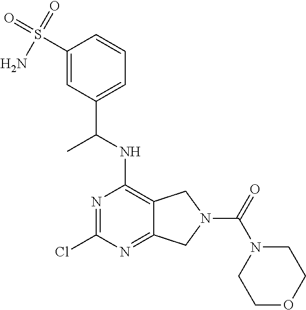 [SMILES: CC(Nc1nc(Cl)nc2c1CN(C(=O)N1CCOCC1)C2)c1cccc(S(N)(=O)=O)c1] |
| Example 536. | 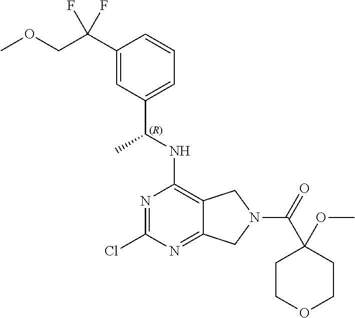 |
| Example 537. | 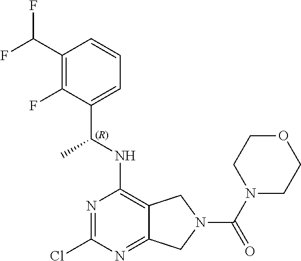 |
| Example 538. | 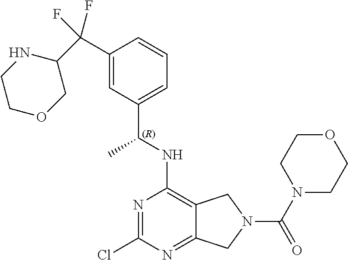 |
| Example 539. | 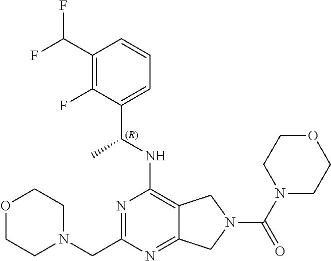 |
| Example 540. | 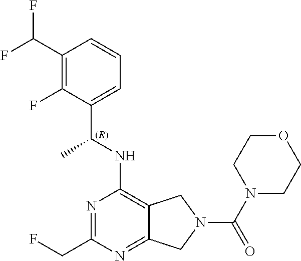 |

The present disclosure provides a compound, and pharmaceutically acceptable salts, solvates, stereoisomers, and tautomers thereof, selected from the group consisting of compounds of Collection 1:

### Collection 1: Certain Compounds of the Present Invention

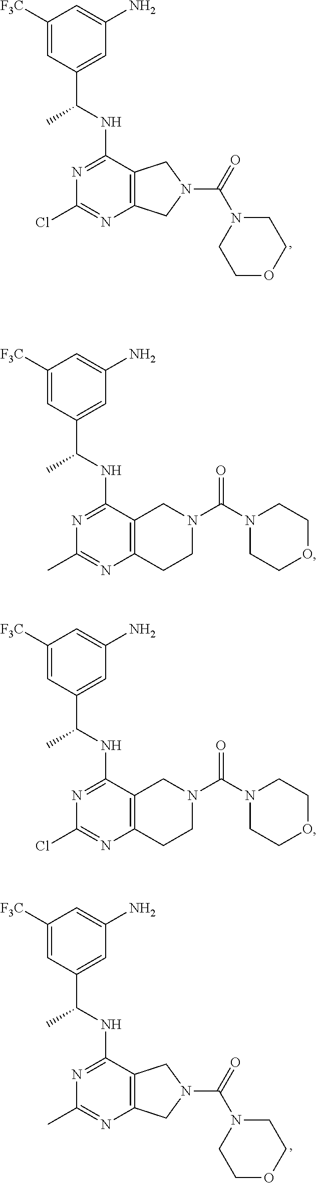 [SMILES: C[C@@H](Nc1nc(Cl)nc2c1CN(C(=O)N1CCOCC1)C2)c1cc(N)cc(C(F)(F)F)c1 ; Cc1nc2c(c(N[C@H](C)c3cc(N)cc(C(F)(F)F)c3)n1)CN(C(=O)N1CCOCC1)CC2 ; C[C@@H](Nc1nc(Cl)nc2c1CN(C(=O)N1CCOCC1)CC2)c1cc(N)cc(C(F)(F)F)c1 ; Cc1nc2c(c(N[C@H](C)c3cc(N)cc(C(F)(F)F)c3)n1)CN(C(=O)N1CCOCC1)C2]

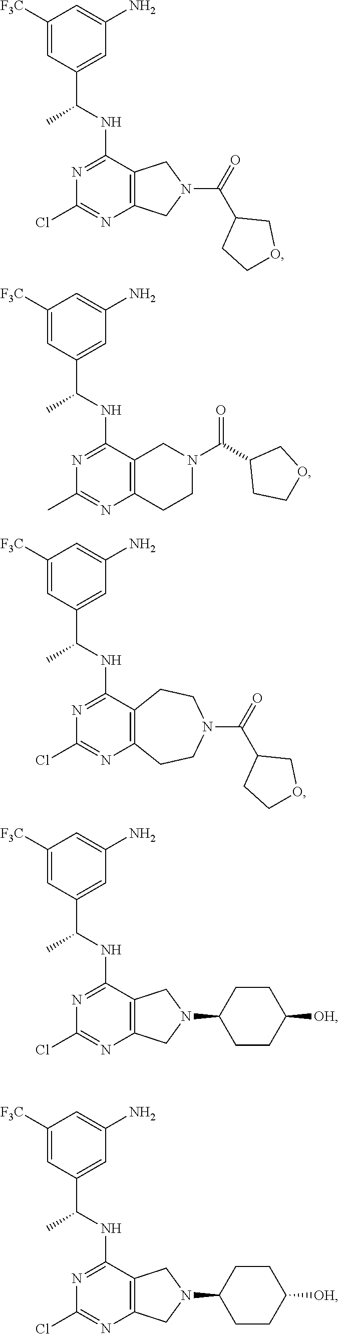

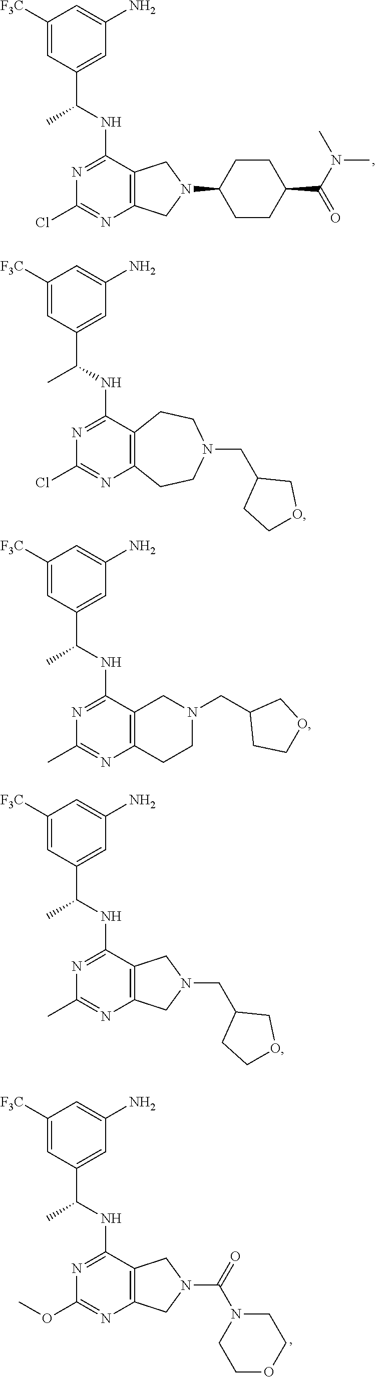

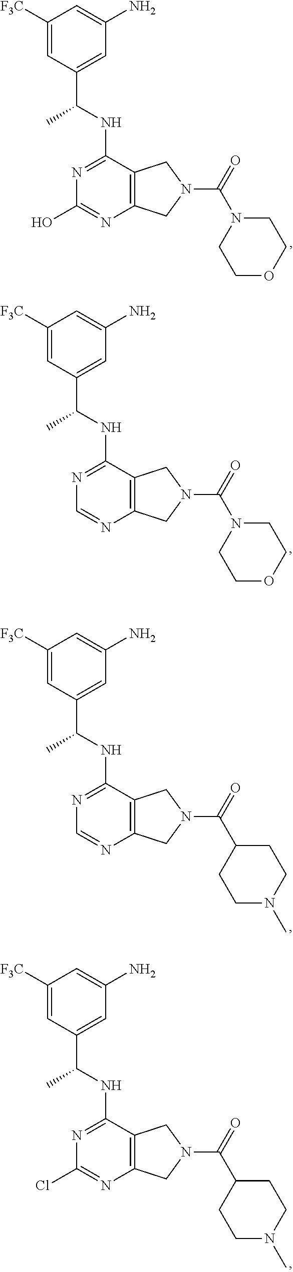 [SMILES: C[C@@H](Nc1nc(O)nc2c1CN(C(=O)N1CCOCC1)C2)c1cc(N)cc(C(F)(F)F)c1 ; C[C@@H](Nc1ncnc2c1CN(C(=O)N1CCOCC1)C2)c1cc(N)cc(C(F)(F)F)c1 ; C[C@@H](Nc1ncnc2c1CN(C(=O)C1CCN(C)CC1)C2)c1cc(N)cc(C(F)(F)F)c1 ; C[C@@H](Nc1nc(Cl)nc2c1CN(C(=O)C1CCN(C)CC1)C2)c1cc(N)cc(C(F)(F)F)c1]

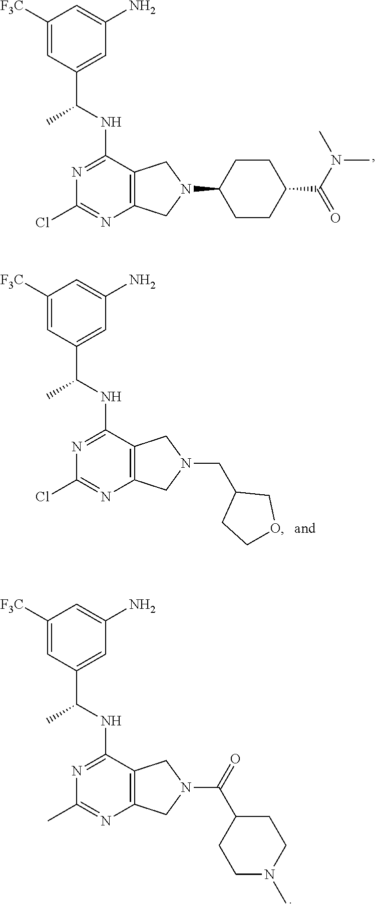 [SMILES: C[C@@H](Nc1nc(Cl)nc2c1CN([C@H]1CC[C@H](C(=O)N(C)C)CC1)C2)c1cc(N)cc(C(F)(F)F)c1 ; C[C@@H](Nc1nc(Cl)nc2c1CN(CC1CCOC1)C2)c1cc(N)cc(C(F)(F)F)c1 ; Cc1nc2c(c(N[C@H](C)c3cc(N)cc(C(F)(F)F)c3)n1)CN(C(=O)C1CCN(C)CC1)C2]

The present disclosure provides a compound, and pharmaceutically acceptable salts, solvates, stereoisomers, and tautomers thereof, selected from the group consisting of compounds of Collection 2:

### Collection 2: Certain Compounds of the Present Invention

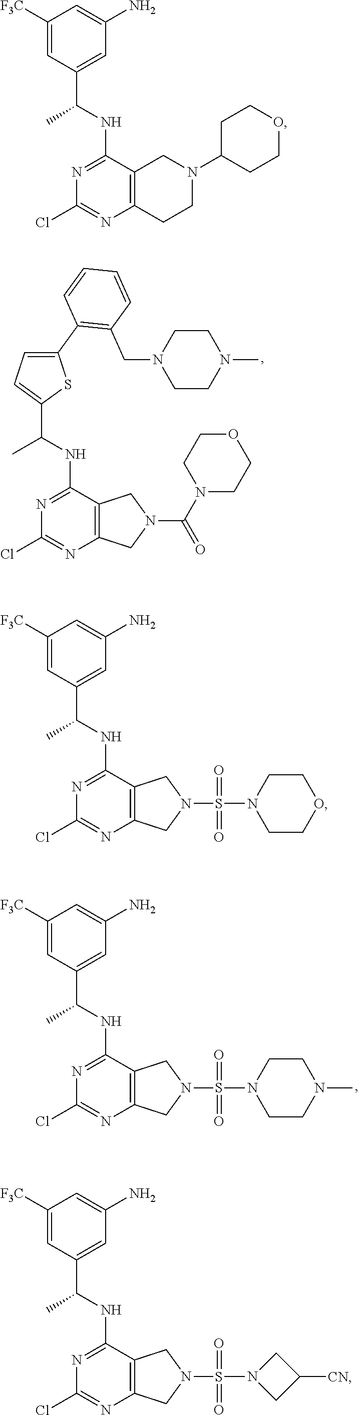 [SMILES: C[C@@H](Nc1nc(Cl)nc2c1CN(C1CCOCC1)CC2)c1cc(N)cc(C(F)(F)F)c1 ; CC(Nc1nc(Cl)nc2c1CN(C(=O)N1CCOCC1)C2)c1ccc(-c2ccccc2CN2CCN(C)CC2)s1 ; C[C@@H](Nc1nc(Cl)nc2c1CN(S(=O)(=O)N1CCOCC1)C2)c1cc(N)cc(C(F)(F)F)c1 ; C[C@@H](Nc1nc(Cl)nc2c1CN(S(=O)(=O)N1CCN(C)CC1)C2)c1cc(N)cc(C(F)(F)F)c1 ; C[C@@H](Nc1nc(Cl)nc2c1CN(S(=O)(=O)N1CC(C#N)C1)C2)c1cc(N)cc(C(F)(F)F)c1]

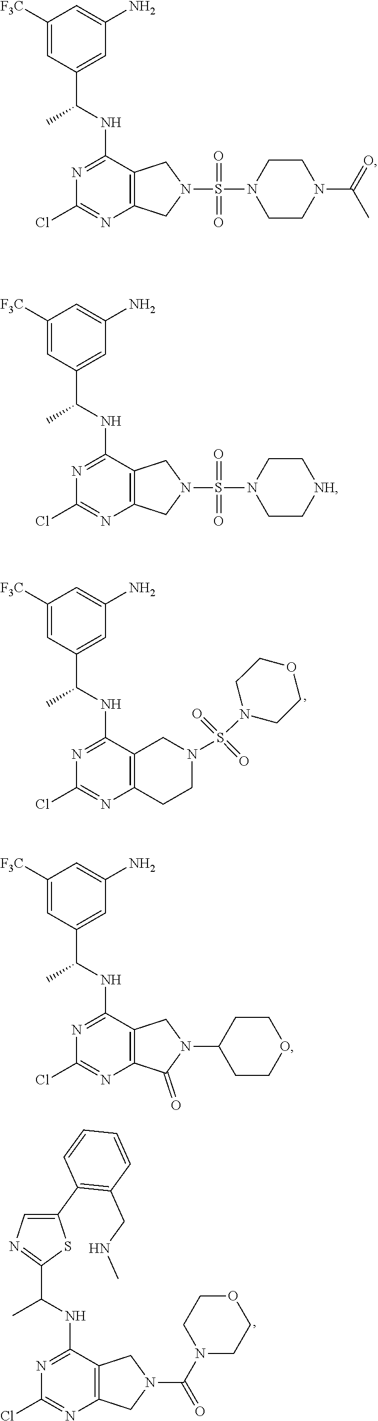 [SMILES: CC(=O)N1CCN(S(=O)(=O)N2Cc3nc(Cl)nc(N[C@H](C)c4cc(N)cc(C(F)(F)F)c4)c3C2)CC1 ; C[C@@H](Nc1nc(Cl)nc2c1CN(S(=O)(=O)N1CCNCC1)C2)c1cc(N)cc(C(F)(F)F)c1 ; C[C@@H](Nc1nc(Cl)nc2c1CN(S(=O)(=O)N1CCOCC1)CC2)c1cc(N)cc(C(F)(F)F)c1 ; C[C@@H](Nc1nc(Cl)nc2c1CN(C1CCOCC1)C2=O)c1cc(N)cc(C(F)(F)F)c1 ; CNCc1ccccc1-c1cnc(C(C)Nc2nc(Cl)nc3c2CN(C(=O)N2CCOCC2)C3)s1]

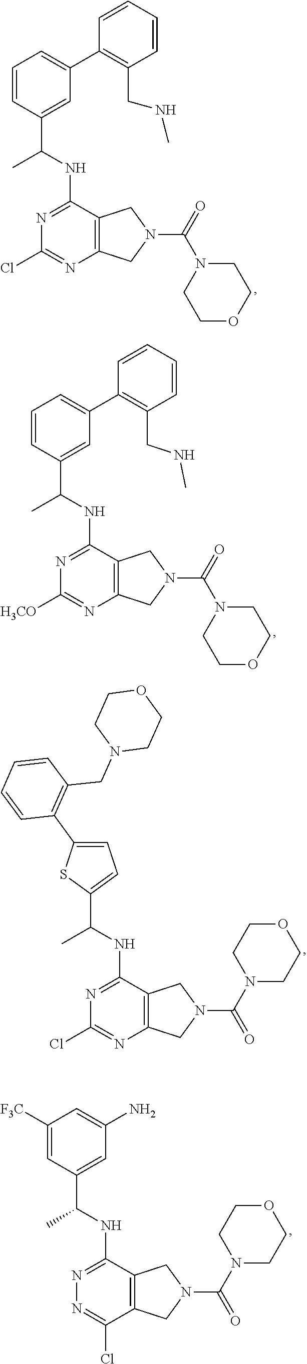 [SMILES: CNCc1ccccc1-c1cccc(C(C)Nc2nc(Cl)nc3c2CN(C(=O)N2CCOCC2)C3)c1 ; CNCc1ccccc1-c1cccc(C(C)Nc2nc(OC)nc3c2CN(C(=O)N2CCOCC2)C3)c1 ; CC(Nc1nc(Cl)nc2c1CN(C(=O)N1CCOCC1)C2)c1ccc(-c2ccccc2CN2CCOCC2)s1 ; C[C@@H](Nc1nnc(Cl)c2c1CN(C(=O)N1CCOCC1)C2)c1cc(N)cc(C(F)(F)F)c1]

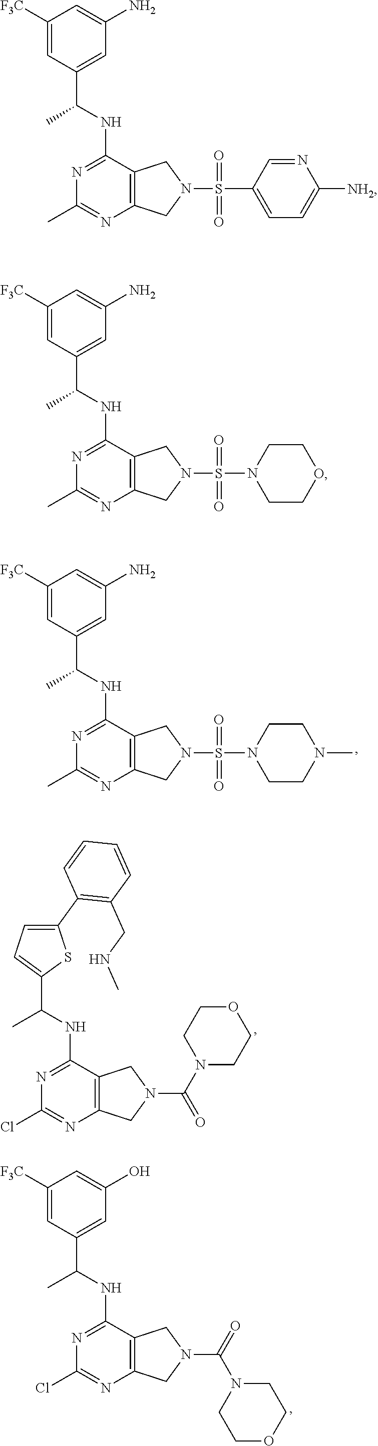

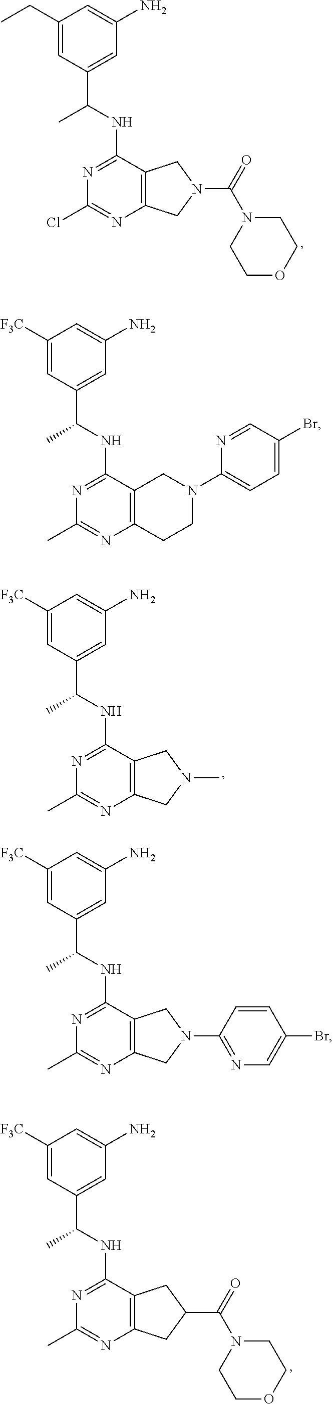 [SMILES: CCc1cc(N)cc(C(C)Nc2nc(Cl)nc3c2CN(C(=O)N2CCOCC2)C3)c1 ; Cc1nc2c(c(N[C@H](C)c3cc(N)cc(C(F)(F)F)c3)n1)CN(c1ccc(Br)cn1)CC2 ; Cc1nc2c(c(N[C@H](C)c3cc(N)cc(C(F)(F)F)c3)n1)CN(C)C2 ; Cc1nc2c(c(N[C@H](C)c3cc(N)cc(C(F)(F)F)c3)n1)CN(c1ccc(Br)cn1)C2 ; Cc1nc2c(c(N[C@H](C)c3cc(N)cc(C(F)(F)F)c3)n1)CC(C(=O)N1CCOCC1)C2]

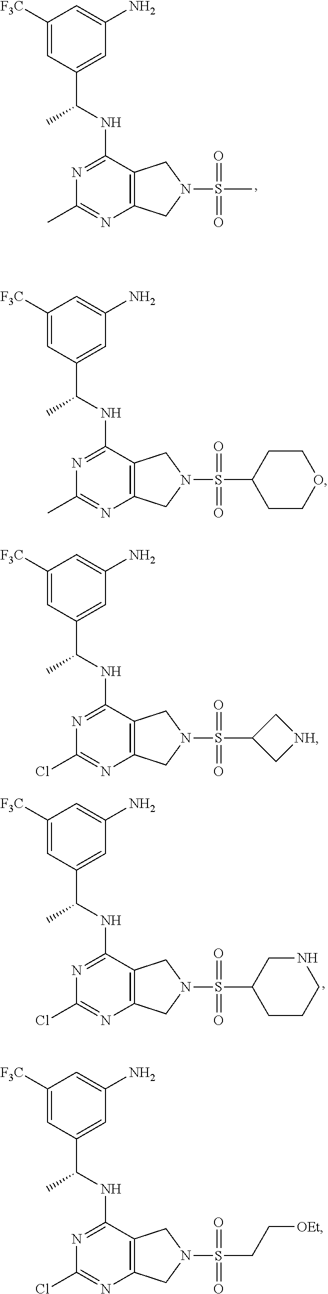

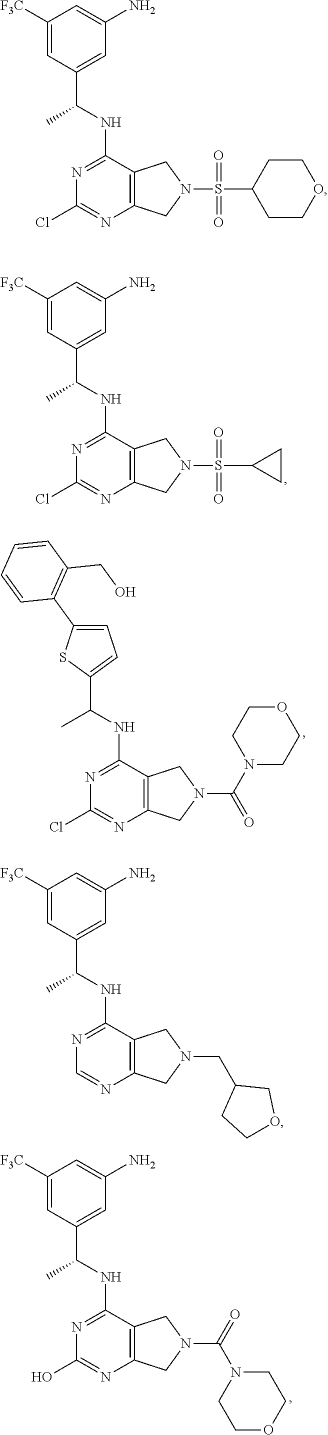

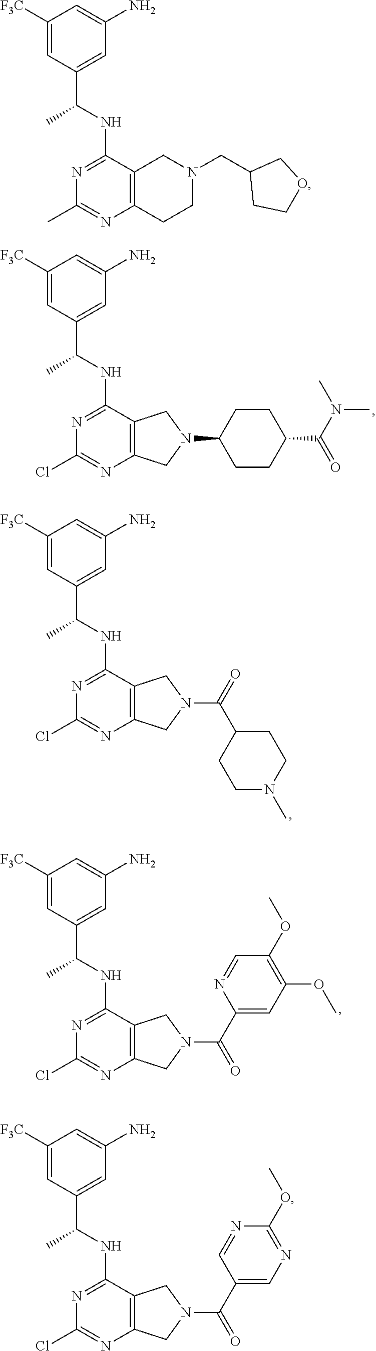

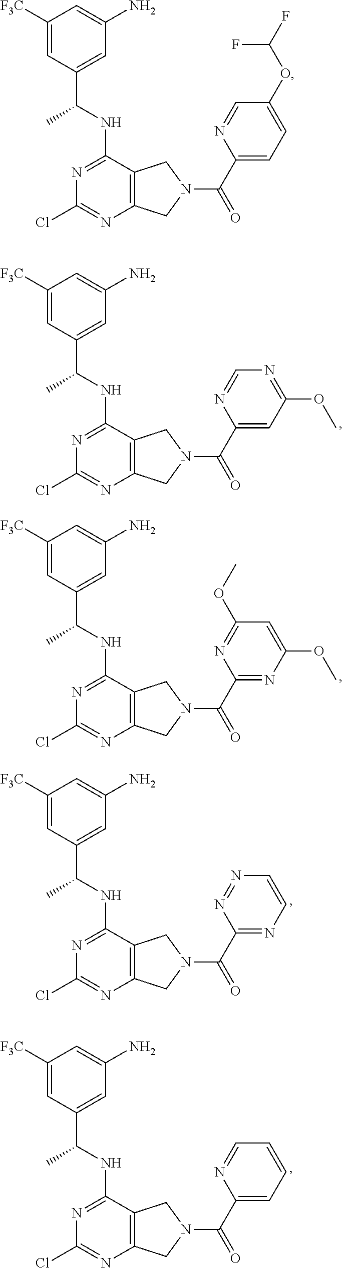

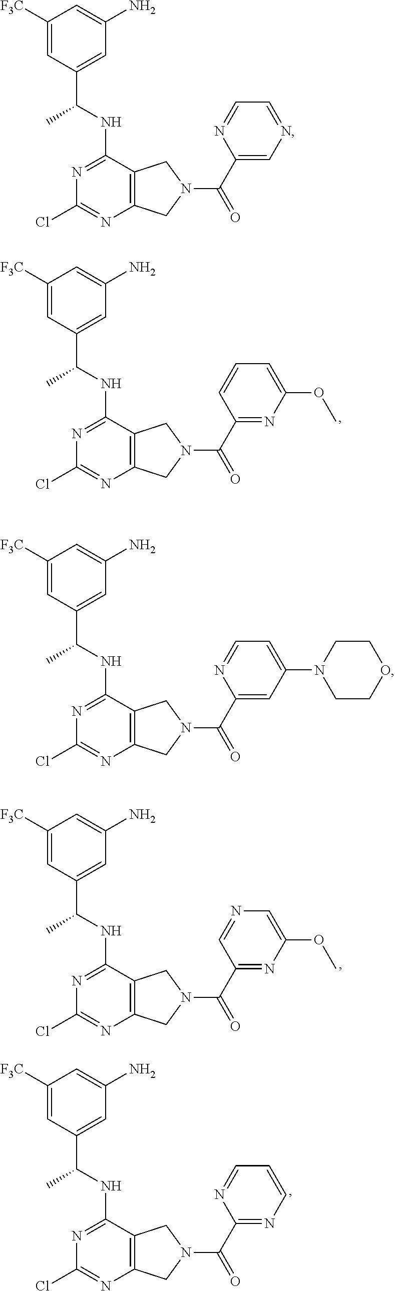 [SMILES: C[C@@H](Nc1nc(Cl)nc2c1CN(C(=O)c1cnccn1)C2)c1cc(N)cc(C(F)(F)F)c1 ; COc1cccc(C(=O)N2Cc3nc(Cl)nc(N[C@H](C)c4cc(N)cc(C(F)(F)F)c4)c3C2)n1 ; C[C@@H](Nc1nc(Cl)nc2c1CN(C(=O)c1cc(N3CCOCC3)ccn1)C2)c1cc(N)cc(C(F)(F)F)c1 ; COc1cncc(C(=O)N2Cc3nc(Cl)nc(N[C@H](C)c4cc(N)cc(C(F)(F)F)c4)c3C2)n1 ; C[C@@H](Nc1nc(Cl)nc2c1CN(C(=O)c1ncccn1)C2)c1cc(N)cc(C(F)(F)F)c1]

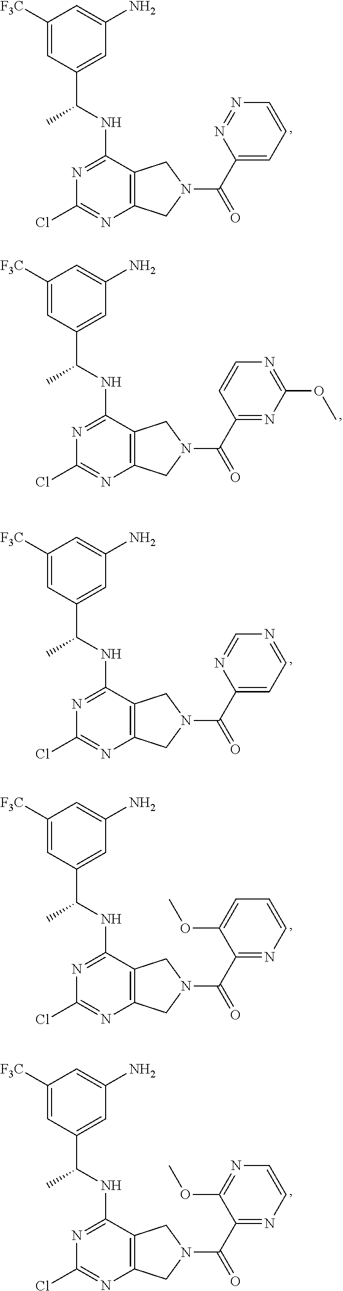 [SMILES: C[C@@H](Nc1nc(Cl)nc2c1CN(C(=O)c1cccnn1)C2)c1cc(N)cc(C(F)(F)F)c1 ; COc1nccc(C(=O)N2Cc3nc(Cl)nc(N[C@H](C)c4cc(N)cc(C(F)(F)F)c4)c3C2)n1 ; C[C@@H](Nc1nc(Cl)nc2c1CN(C(=O)c1ccncn1)C2)c1cc(N)cc(C(F)(F)F)c1 ; COc1cccnc1C(=O)N1Cc2nc(Cl)nc(N[C@H](C)c3cc(N)cc(C(F)(F)F)c3)c2C1 ; COc1nccnc1C(=O)N1Cc2nc(Cl)nc(N[C@H](C)c3cc(N)cc(C(F)(F)F)c3)c2C1]

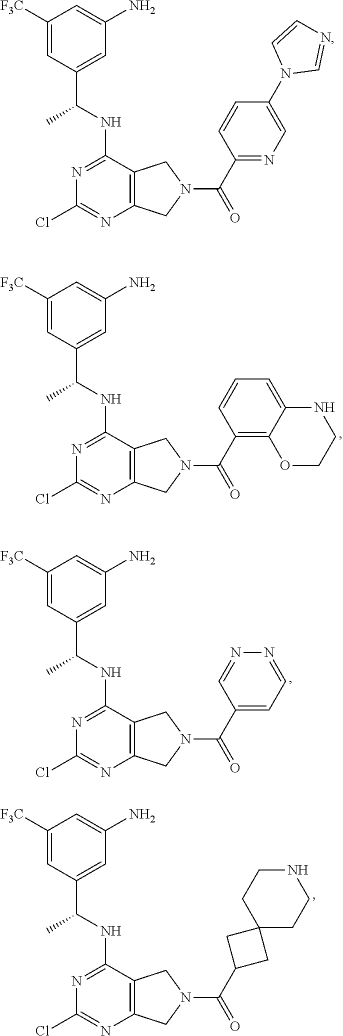 [SMILES: C[C@@H](Nc1nc(Cl)nc2c1CN(C(=O)c1ccc(-n3ccnc3)cn1)C2)c1cc(N)cc(C(F)(F)F)c1 ; C[C@@H](Nc1nc(Cl)nc2c1CN(C(=O)c1cccc3c1OCCN3)C2)c1cc(N)cc(C(F)(F)F)c1 ; C[C@@H](Nc1nc(Cl)nc2c1CN(C(=O)c1ccnnc1)C2)c1cc(N)cc(C(F)(F)F)c1 ; C[C@@H](Nc1nc(Cl)nc2c1CN(C(=O)C1CC3(CCNCC3)C1)C2)c1cc(N)cc(C(F)(F)F)c1]

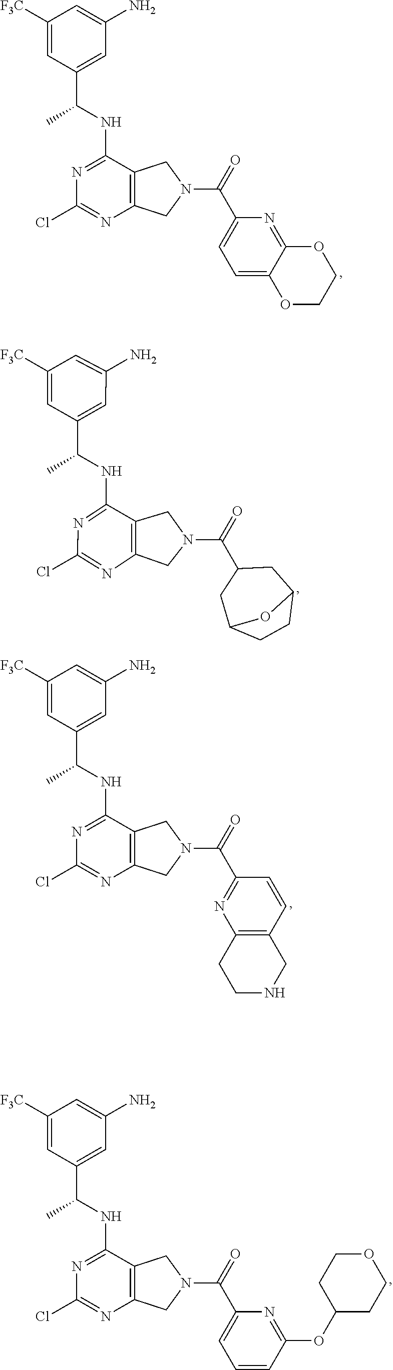 [SMILES: C[C@@H](Nc1nc(Cl)nc2c1CN(C(=O)c1ccc3c(n1)OCCO3)C2)c1cc(N)cc(C(F)(F)F)c1 ; C[C@@H](Nc1nc(Cl)nc2c1CN(C(=O)C1CC3CCC(C1)O3)C2)c1cc(N)cc(C(F)(F)F)c1 ; C[C@@H](Nc1nc(Cl)nc2c1CN(C(=O)c1ccc3c(n1)CCNC3)C2)c1cc(N)cc(C(F)(F)F)c1 ; C[C@@H](Nc1nc(Cl)nc2c1CN(C(=O)c1cccc(OC3CCOCC3)n1)C2)c1cc(N)cc(C(F)(F)F)c1]

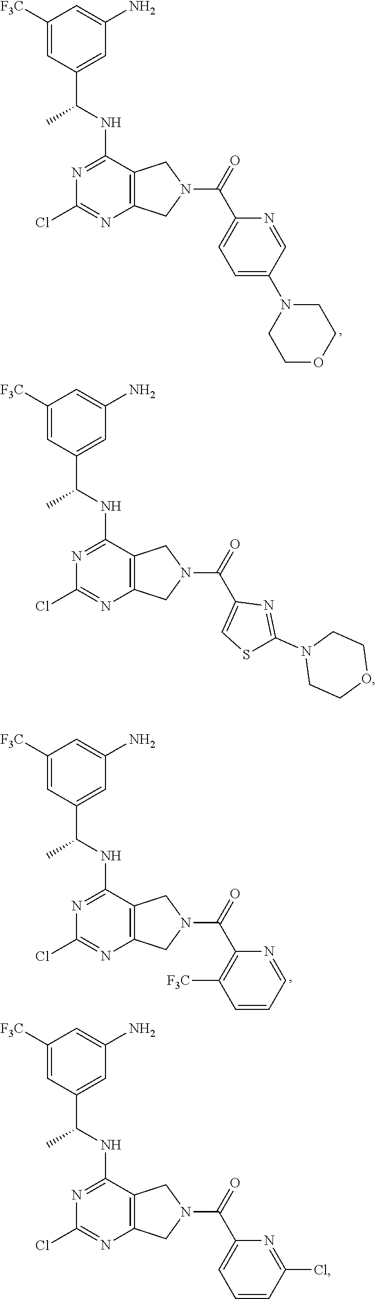 [SMILES: C[C@@H](Nc1nc(Cl)nc2c1CN(C(=O)c1ccc(N3CCOCC3)cn1)C2)c1cc(N)cc(C(F)(F)F)c1 ; C[C@@H](Nc1nc(Cl)nc2c1CN(C(=O)c1csc(N3CCOCC3)n1)C2)c1cc(N)cc(C(F)(F)F)c1 ; C[C@@H](Nc1nc(Cl)nc2c1CN(C(=O)c1ncccc1C(F)(F)F)C2)c1cc(N)cc(C(F)(F)F)c1 ; C[C@@H](Nc1nc(Cl)nc2c1CN(C(=O)c1cccc(Cl)n1)C2)c1cc(N)cc(C(F)(F)F)c1]

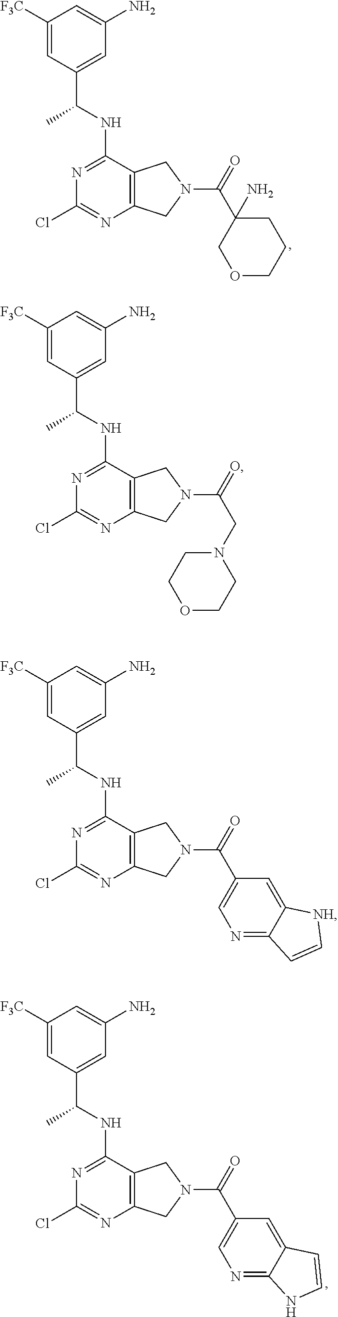 [SMILES: C[C@@H](Nc1nc(Cl)nc2c1CN(C(=O)C1(N)CCCOC1)C2)c1cc(N)cc(C(F)(F)F)c1 ; C[C@@H](Nc1nc(Cl)nc2c1CN(C(=O)CN1CCOCC1)C2)c1cc(N)cc(C(F)(F)F)c1 ; C[C@@H](Nc1nc(Cl)nc2c1CN(C(=O)c1cnc3cc[nH]c3c1)C2)c1cc(N)cc(C(F)(F)F)c1 ; C[C@@H](Nc1nc(Cl)nc2c1CN(C(=O)c1cnc3[nH]ccc3c1)C2)c1cc(N)cc(C(F)(F)F)c1]

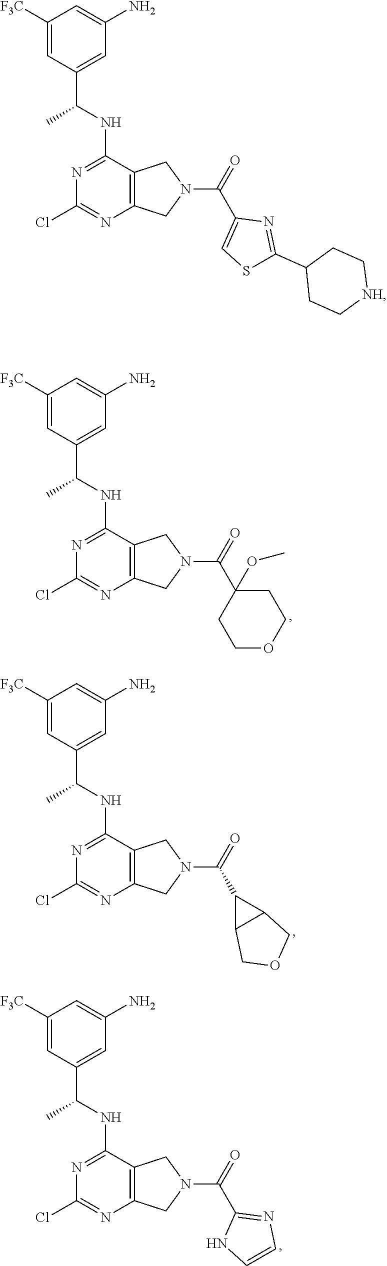

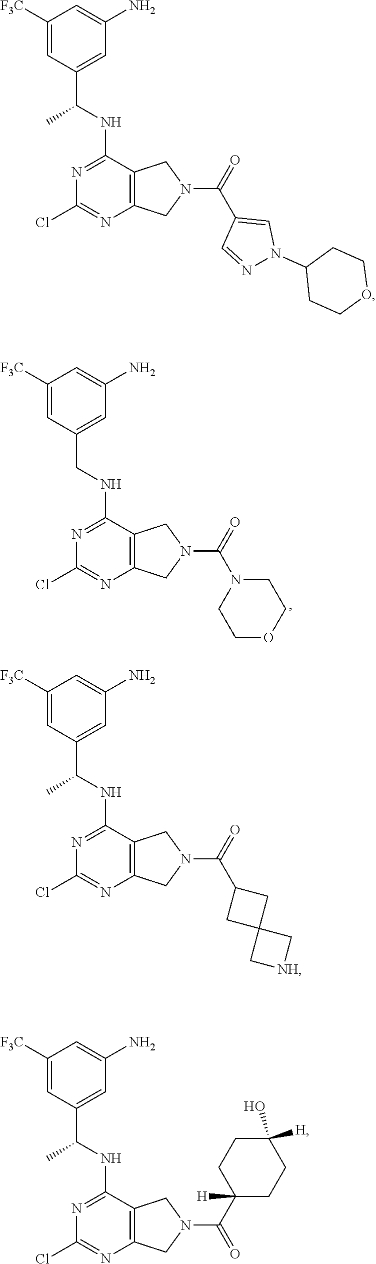

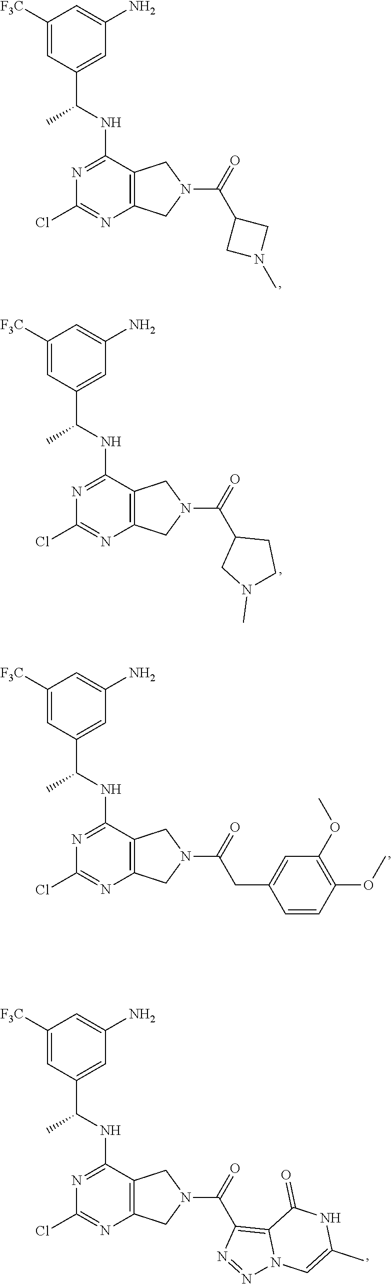 [SMILES: C[C@@H](Nc1nc(Cl)nc2c1CN(C(=O)C1CN(C)C1)C2)c1cc(N)cc(C(F)(F)F)c1 ; C[C@@H](Nc1nc(Cl)nc2c1CN(C(=O)C1CCN(C)C1)C2)c1cc(N)cc(C(F)(F)F)c1 ; COc1ccc(CC(=O)N2Cc3nc(Cl)nc(N[C@H](C)c4cc(N)cc(C(F)(F)F)c4)c3C2)cc1OC ; Cc1cn2nnc(C(=O)N3Cc4nc(Cl)nc(N[C@H](C)c5cc(N)cc(C(F)(F)F)c5)c4C3)c2c(=O)[nH]1]

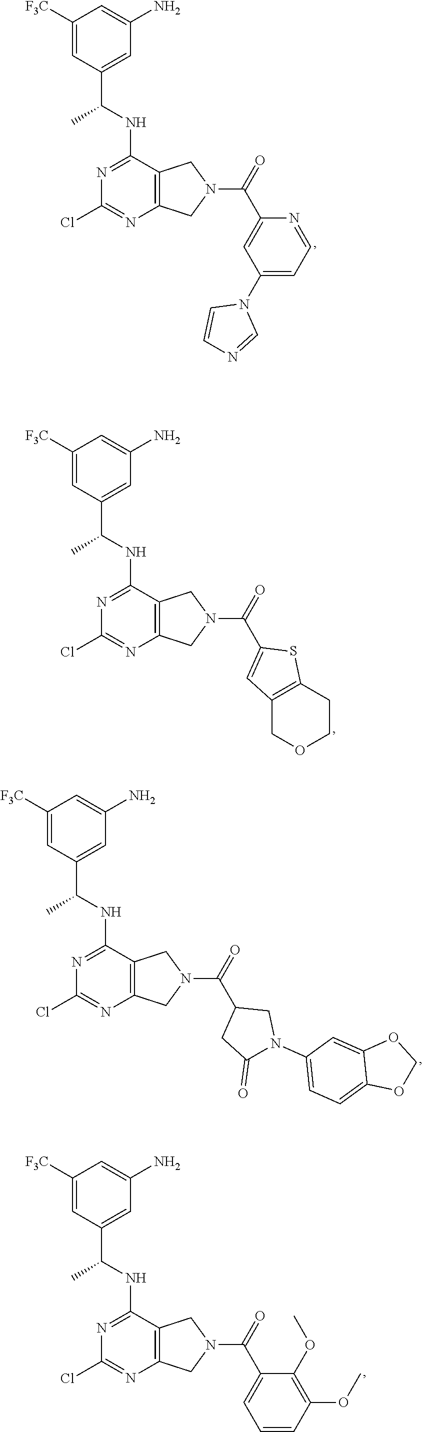 [SMILES: C[C@@H](Nc1nc(Cl)nc2c1CN(C(=O)c1cc(-n3ccnc3)ccn1)C2)c1cc(N)cc(C(F)(F)F)c1 ; C[C@@H](Nc1nc(Cl)nc2c1CN(C(=O)c1cc3c(s1)CCOC3)C2)c1cc(N)cc(C(F)(F)F)c1 ; C[C@@H](Nc1nc(Cl)nc2c1CN(C(=O)C1CC(=O)N(c3ccc4c(c3)OCO4)C1)C2)c1cc(N)cc(C(F)(F)F)c1 ; COc1cccc(C(=O)N2Cc3nc(Cl)nc(N[C@H](C)c4cc(N)cc(C(F)(F)F)c4)c3C2)c1OC]

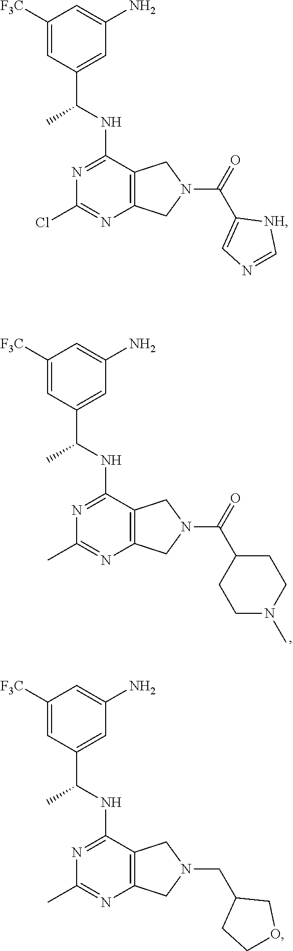 [SMILES: C[C@@H](Nc1nc(Cl)nc2c1CN(C(=O)c1cnc[nH]1)C2)c1cc(N)cc(C(F)(F)F)c1 ; Cc1nc2c(c(N[C@H](C)c3cc(N)cc(C(F)(F)F)c3)n1)CN(C(=O)C1CCN(C)CC1)C2 ; Cc1nc2c(c(N[C@H](C)c3cc(N)cc(C(F)(F)F)c3)n1)CN(CC1CCOC1)C2]

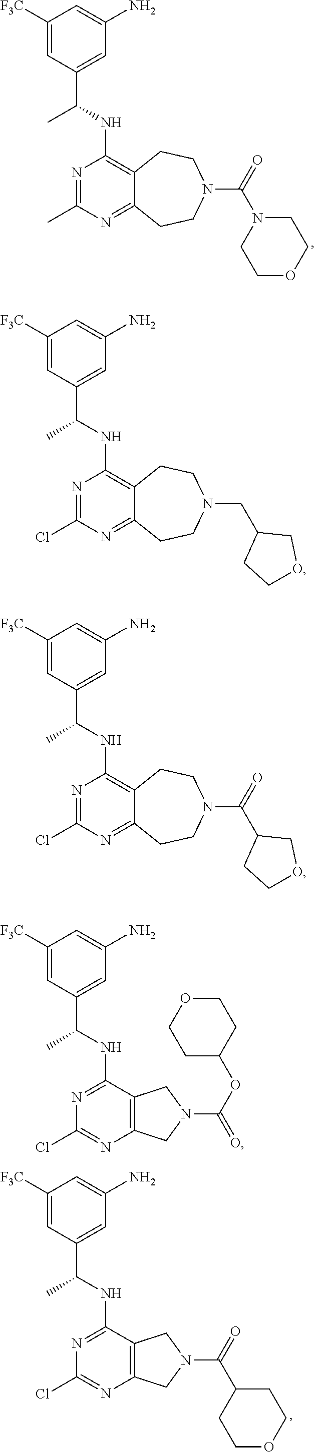

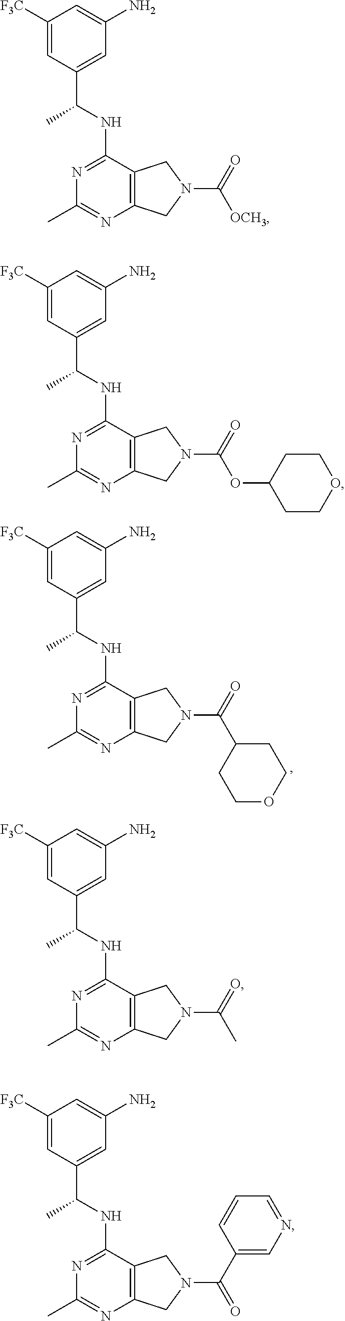 [SMILES: COC(=O)N1Cc2nc(C)nc(N[C@H](C)c3cc(N)cc(C(F)(F)F)c3)c2C1 ; Cc1nc2c(c(N[C@H](C)c3cc(N)cc(C(F)(F)F)c3)n1)CN(C(=O)OC1CCOCC1)C2 ; Cc1nc2c(c(N[C@H](C)c3cc(N)cc(C(F)(F)F)c3)n1)CN(C(=O)C1CCOCC1)C2 ; CC(=O)N1Cc2nc(C)nc(N[C@H](C)c3cc(N)cc(C(F)(F)F)c3)c2C1 ; Cc1nc2c(c(N[C@H](C)c3cc(N)cc(C(F)(F)F)c3)n1)CN(C(=O)c1cccnc1)C2]

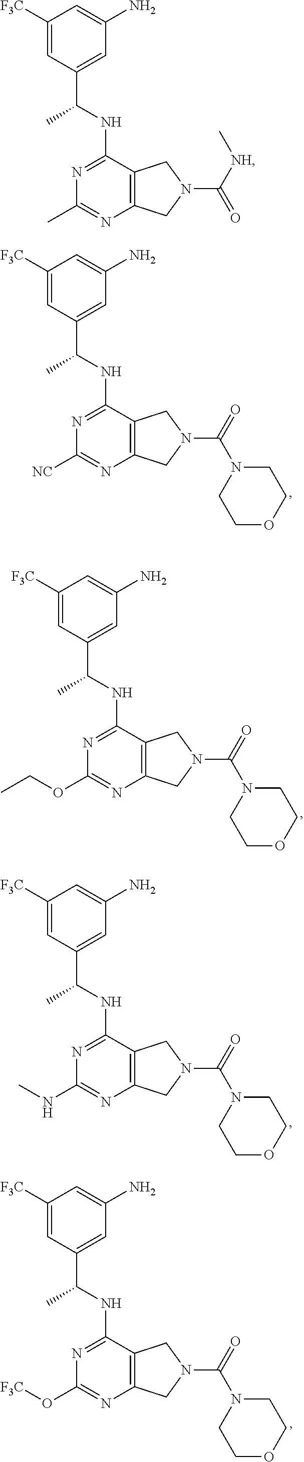

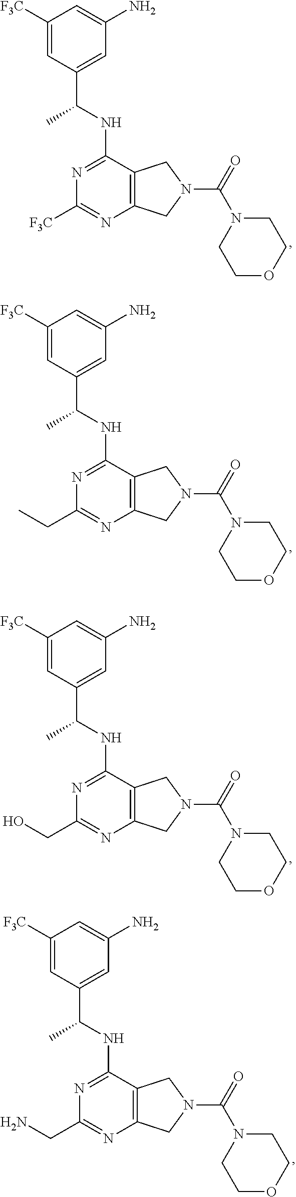 [SMILES: C[C@@H](Nc1nc(C(F)(F)F)nc2c1CN(C(=O)N1CCOCC1)C2)c1cc(N)cc(C(F)(F)F)c1 ; CCc1nc2c(c(N[C@H](C)c3cc(N)cc(C(F)(F)F)c3)n1)CN(C(=O)N1CCOCC1)C2 ; C[C@@H](Nc1nc(CO)nc2c1CN(C(=O)N1CCOCC1)C2)c1cc(N)cc(C(F)(F)F)c1 ; C[C@@H](Nc1nc(CN)nc2c1CN(C(=O)N1CCOCC1)C2)c1cc(N)cc(C(F)(F)F)c1]

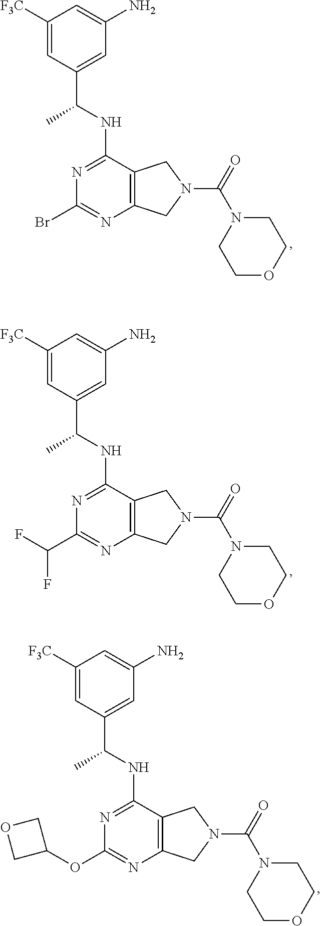 [SMILES: C[C@@H](Nc1nc(Br)nc2c1CN(C(=O)N1CCOCC1)C2)c1cc(N)cc(C(F)(F)F)c1 ; C[C@@H](Nc1nc(C(F)F)nc2c1CN(C(=O)N1CCOCC1)C2)c1cc(N)cc(C(F)(F)F)c1 ; C[C@@H](Nc1nc(OC2COC2)nc2c1CN(C(=O)N1CCOCC1)C2)c1cc(N)cc(C(F)(F)F)c1]

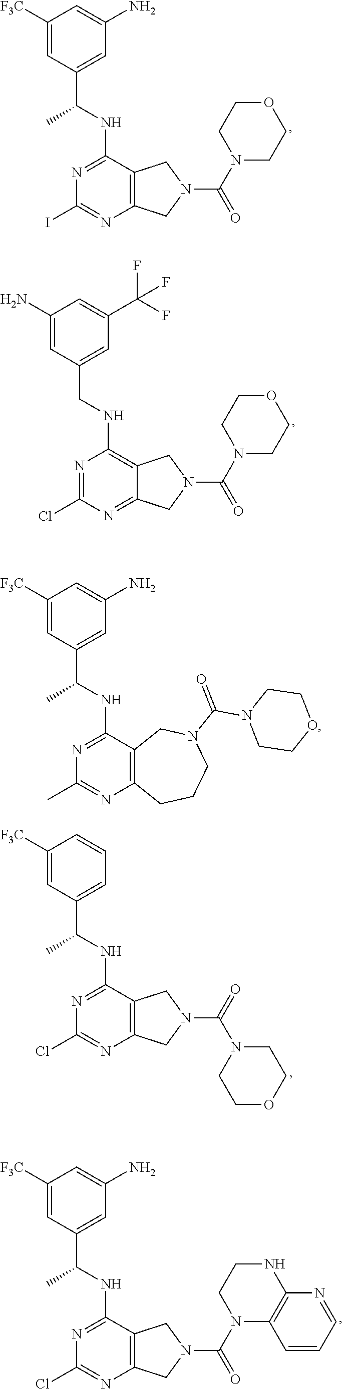

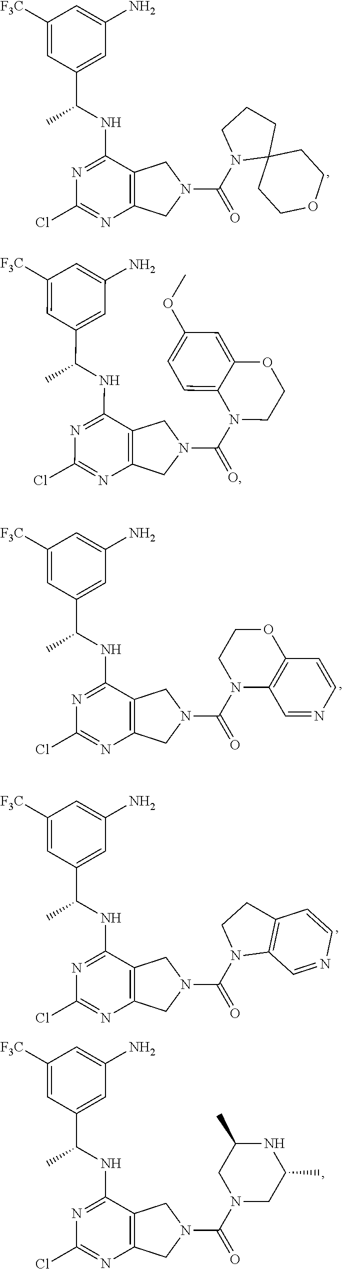

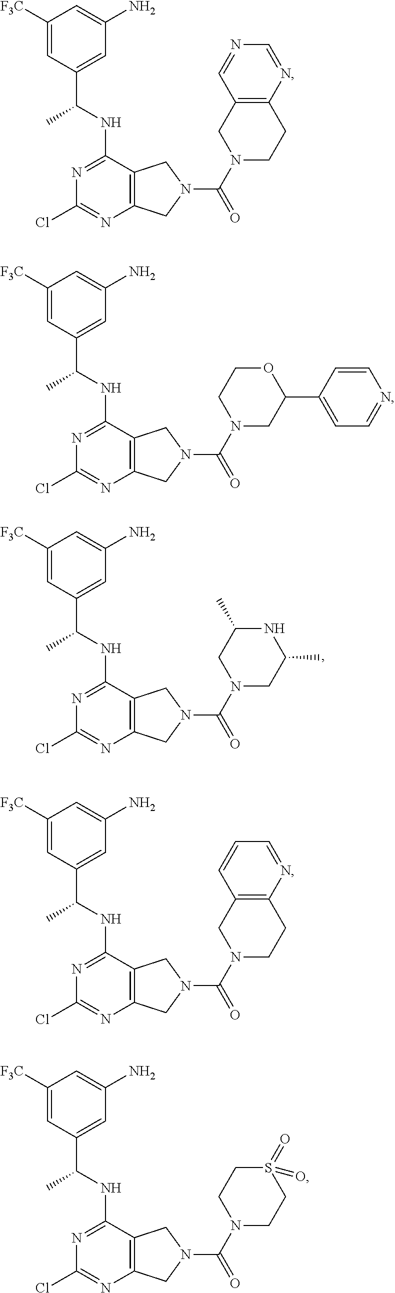 [SMILES: C[C@@H](Nc1nc(Cl)nc2c1CN(C(=O)N1CCc3ncncc3C1)C2)c1cc(N)cc(C(F)(F)F)c1 ; C[C@@H](Nc1nc(Cl)nc2c1CN(C(=O)N1CCOC(c3ccncc3)C1)C2)c1cc(N)cc(C(F)(F)F)c1 ; C[C@@H]1CN(C(=O)N2Cc3nc(Cl)nc(N[C@H](C)c4cc(N)cc(C(F)(F)F)c4)c3C2)C[C@H](C)N1 ; C[C@@H](Nc1nc(Cl)nc2c1CN(C(=O)N1CCc3ncccc3C1)C2)c1cc(N)cc(C(F)(F)F)c1 ; C[C@@H](Nc1nc(Cl)nc2c1CN(C(=O)N1CCS(=O)(=O)CC1)C2)c1cc(N)cc(C(F)(F)F)c1]

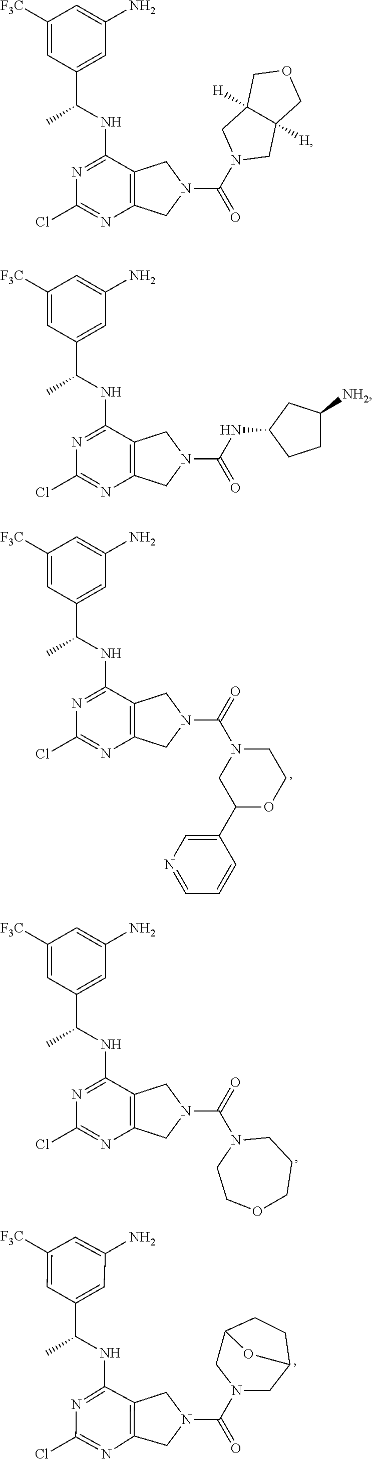

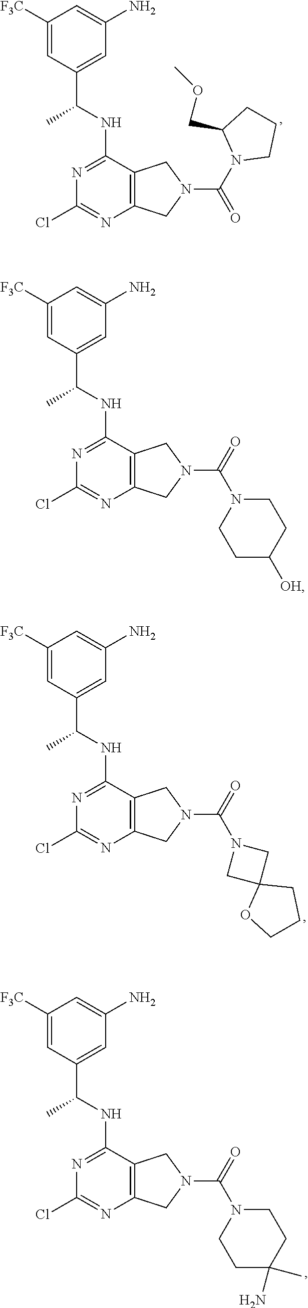 [SMILES: COC[C@H]1CCCN1C(=O)N1Cc2nc(Cl)nc(N[C@H](C)c3cc(N)cc(C(F)(F)F)c3)c2C1 ; C[C@@H](Nc1nc(Cl)nc2c1CN(C(=O)N1CCC(O)CC1)C2)c1cc(N)cc(C(F)(F)F)c1 ; C[C@@H](Nc1nc(Cl)nc2c1CN(C(=O)N1CC3(CCCO3)C1)C2)c1cc(N)cc(C(F)(F)F)c1 ; C[C@@H](Nc1nc(Cl)nc2c1CN(C(=O)N1CCC(C)(N)CC1)C2)c1cc(N)cc(C(F)(F)F)c1]

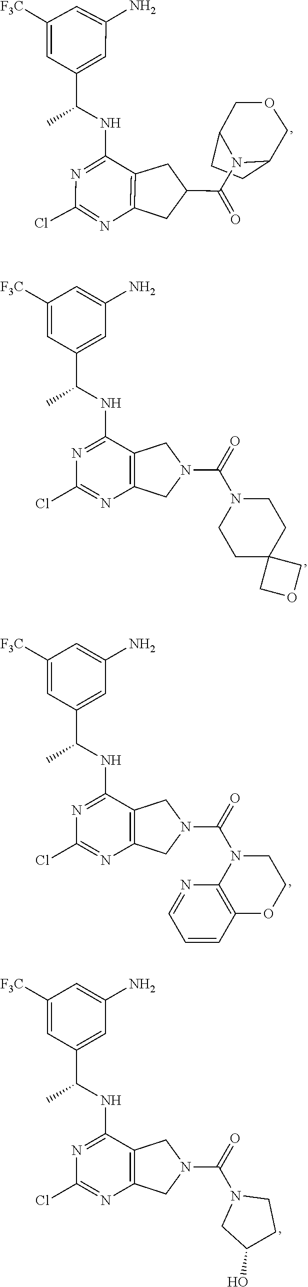 [SMILES: C[C@@H](Nc1nc(Cl)nc2c1CC(C(=O)N1C3CCC1COC3)C2)c1cc(N)cc(C(F)(F)F)c1 ; C[C@@H](Nc1nc(Cl)nc2c1CN(C(=O)N1CCC3(CC1)COC3)C2)c1cc(N)cc(C(F)(F)F)c1 ; C[C@@H](Nc1nc(Cl)nc2c1CN(C(=O)N1CCOc3cccnc31)C2)c1cc(N)cc(C(F)(F)F)c1 ; C[C@@H](Nc1nc(Cl)nc2c1CN(C(=O)N1CC[C@H](O)C1)C2)c1cc(N)cc(C(F)(F)F)c1]

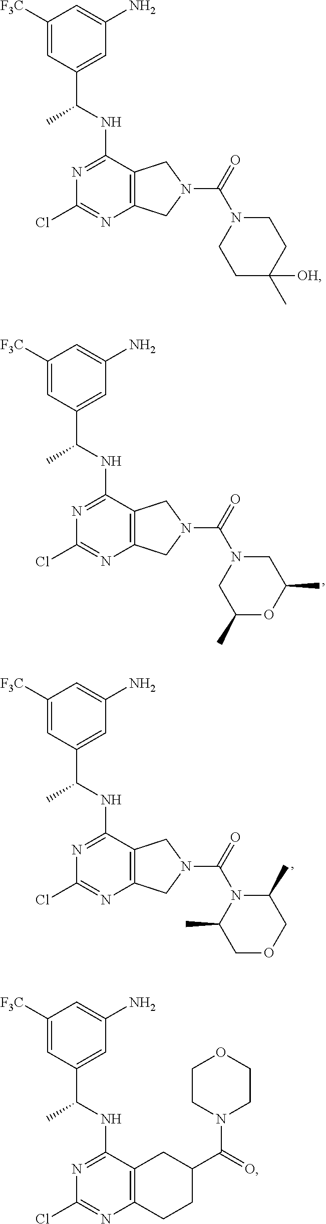 [SMILES: C[C@@H](Nc1nc(Cl)nc2c1CN(C(=O)N1CCC(C)(O)CC1)C2)c1cc(N)cc(C(F)(F)F)c1 ; C[C@@H]1CN(C(=O)N2Cc3nc(Cl)nc(N[C@H](C)c4cc(N)cc(C(F)(F)F)c4)c3C2)C[C@H](C)O1 ; C[C@@H]1COC[C@H](C)N1C(=O)N1Cc2nc(Cl)nc(N[C@H](C)c3cc(N)cc(C(F)(F)F)c3)c2C1 ; C[C@@H](Nc1nc(Cl)nc2c1CC(C(=O)N1CCOCC1)CC2)c1cc(N)cc(C(F)(F)F)c1]

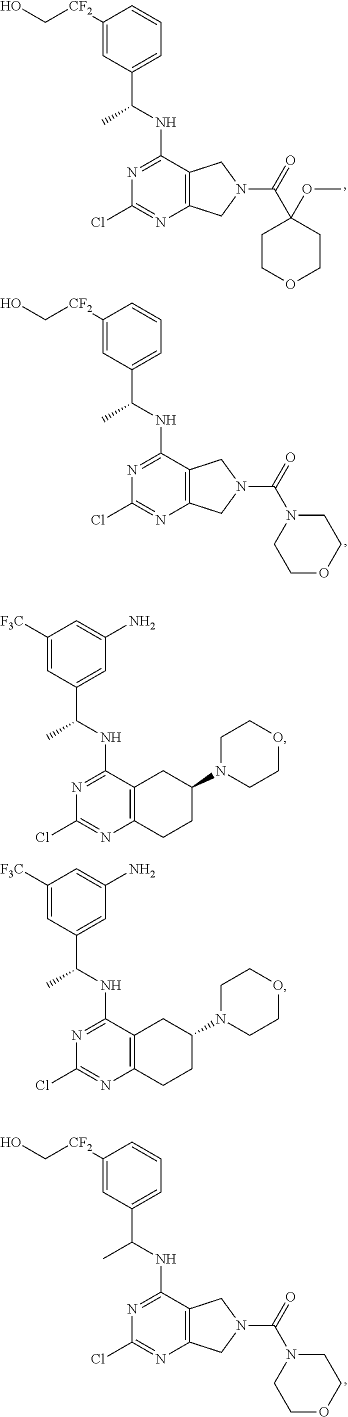

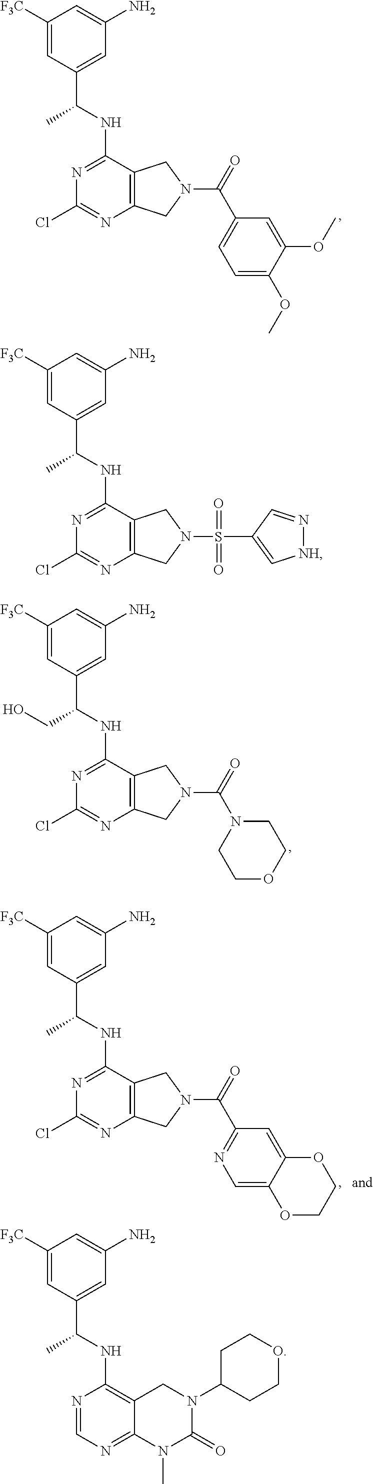 [SMILES: COc1ccc(C(=O)N2Cc3nc(Cl)nc(N[C@H](C)c4cc(N)cc(C(F)(F)F)c4)c3C2)cc1OC ; C[C@@H](Nc1nc(Cl)nc2c1CN(S(=O)(=O)c1cn[nH]c1)C2)c1cc(N)cc(C(F)(F)F)c1 ; Nc1cc([C@@H](CO)Nc2nc(Cl)nc3c2CN(C(=O)N2CCOCC2)C3)cc(C(F)(F)F)c1 ; C[C@@H](Nc1nc(Cl)nc2c1CN(C(=O)c1cc3c(cn1)OCCO3)C2)c1cc(N)cc(C(F)(F)F)c1 ; C[C@@H](Nc1ncnc2c1CN(C1CCOCC1)C(=O)N2C)c1cc(N)cc(C(F)(F)F)c1]

The present disclosure provides a compound, and pharmaceutically acceptable salts, solvates, stereoisomers, and tautomers thereof, selected from the group consisting of compounds of Collection 3:

### Collection 3: Certain Compounds of the Present Invention

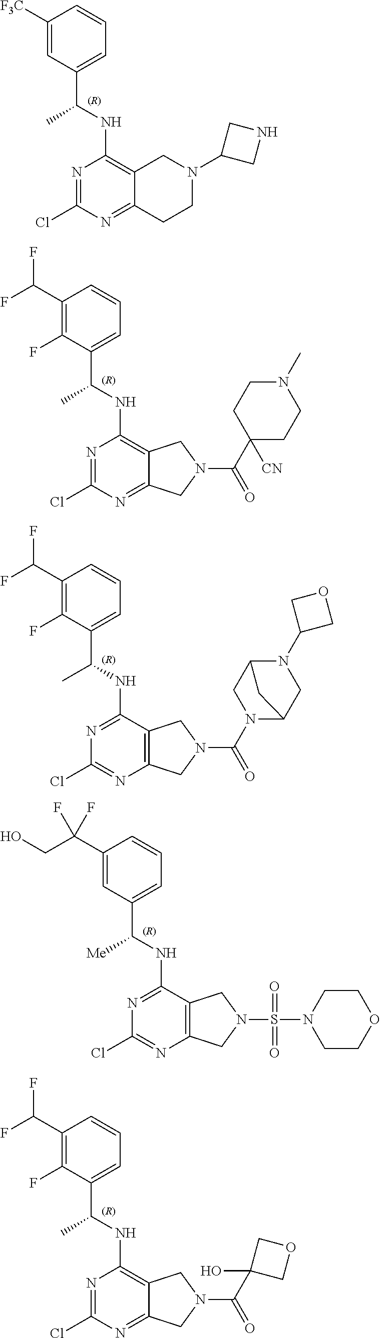

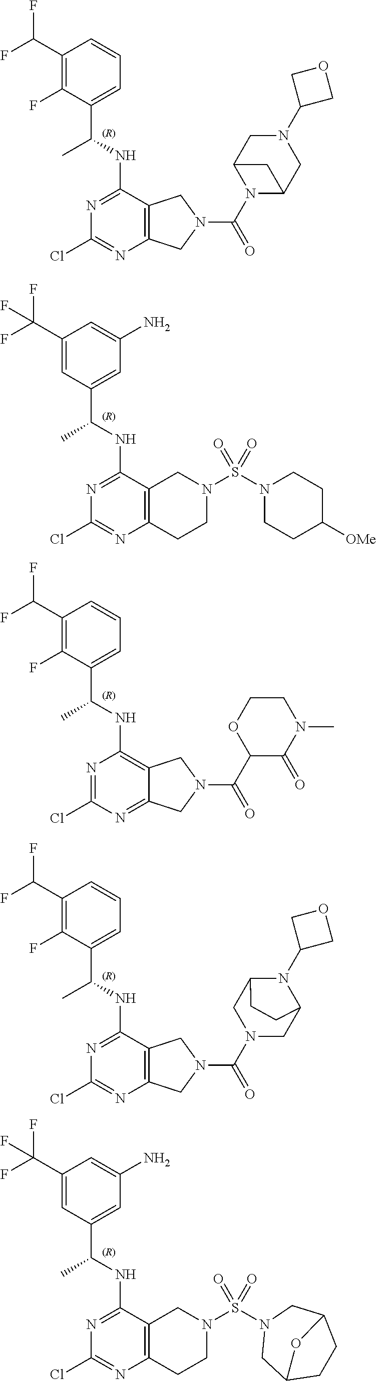

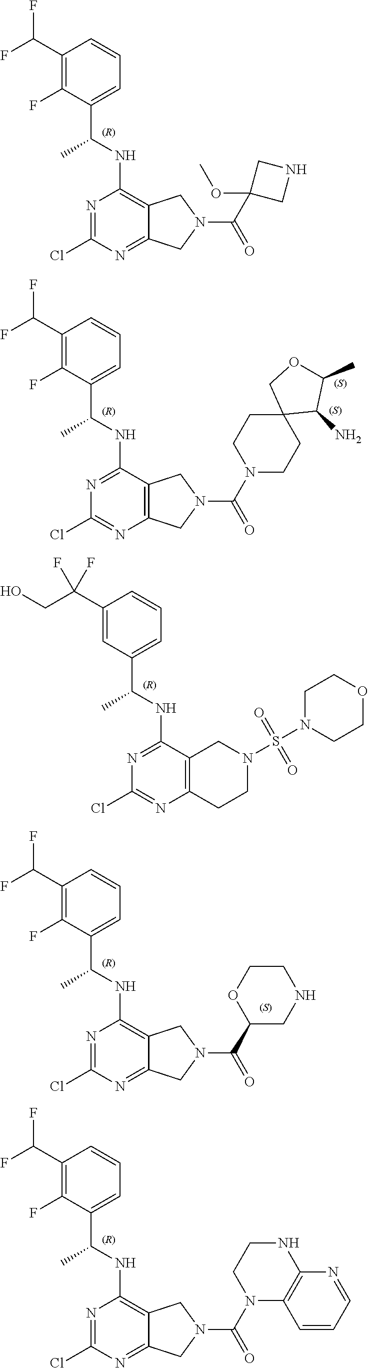

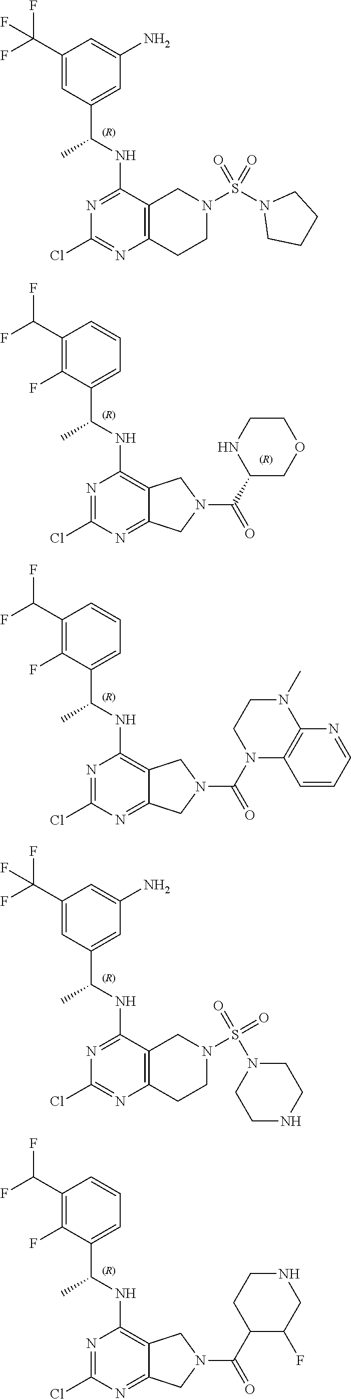

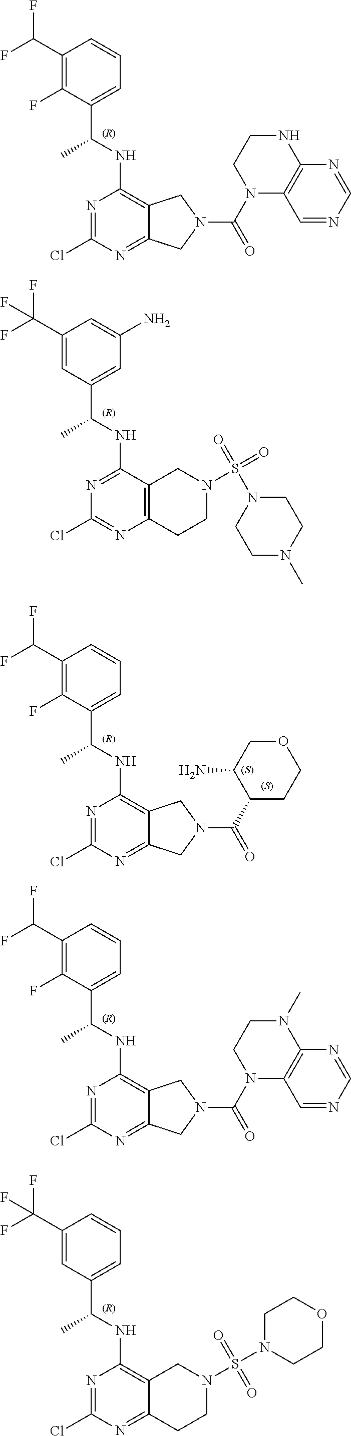

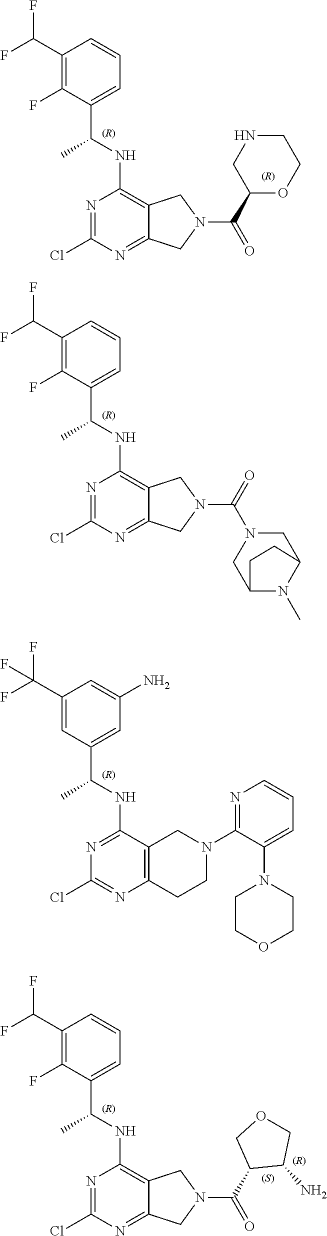

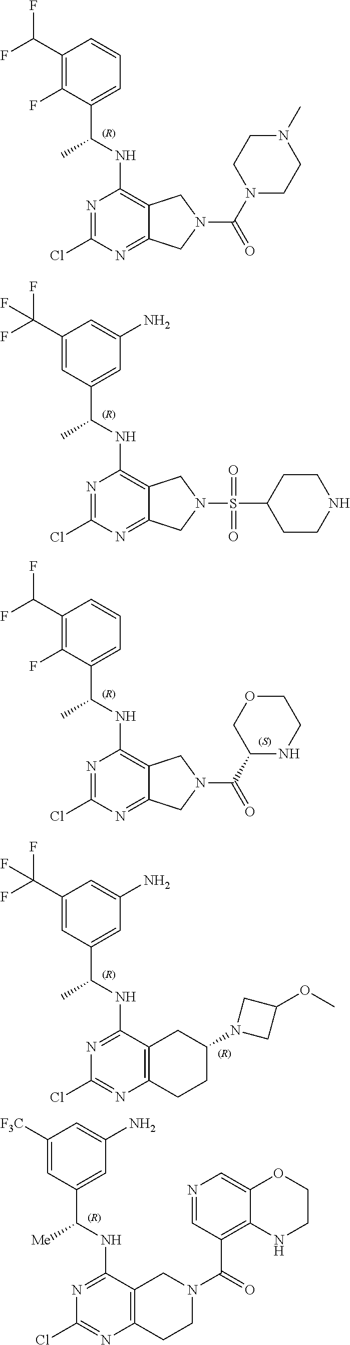

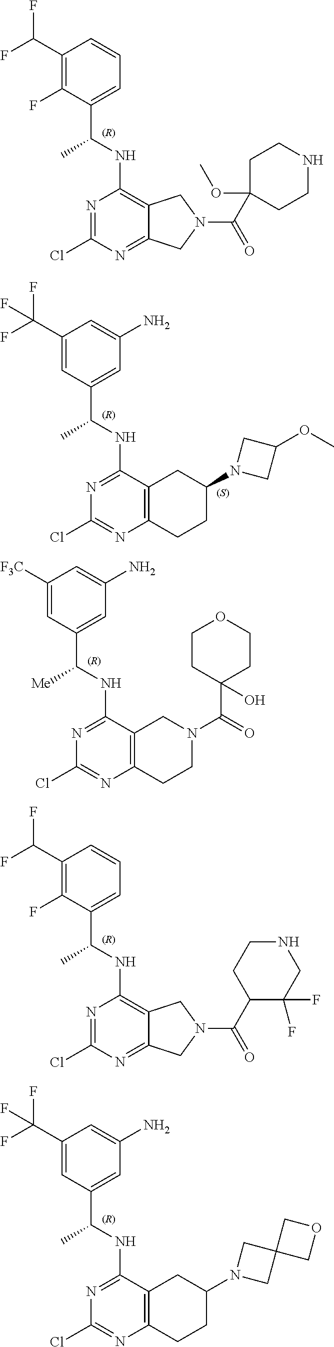

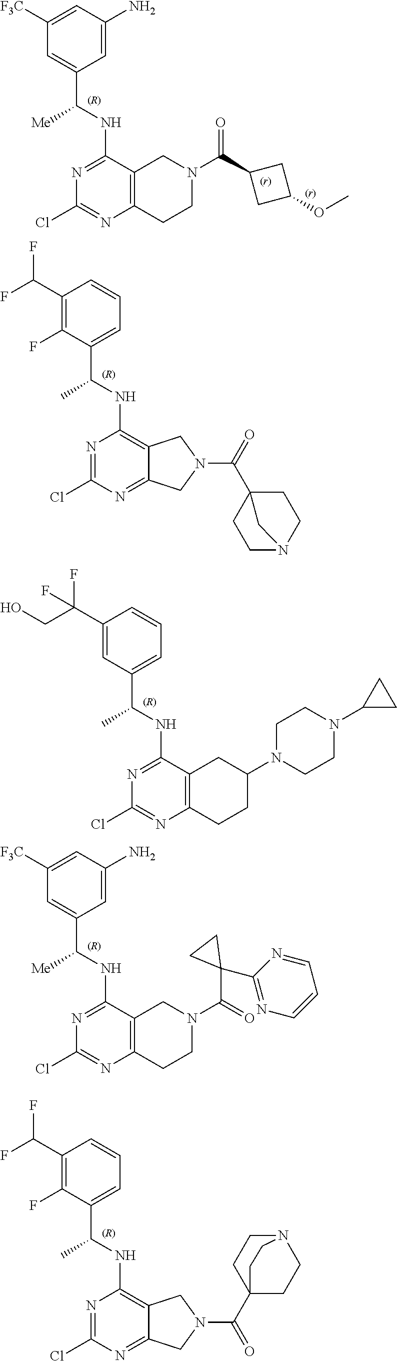

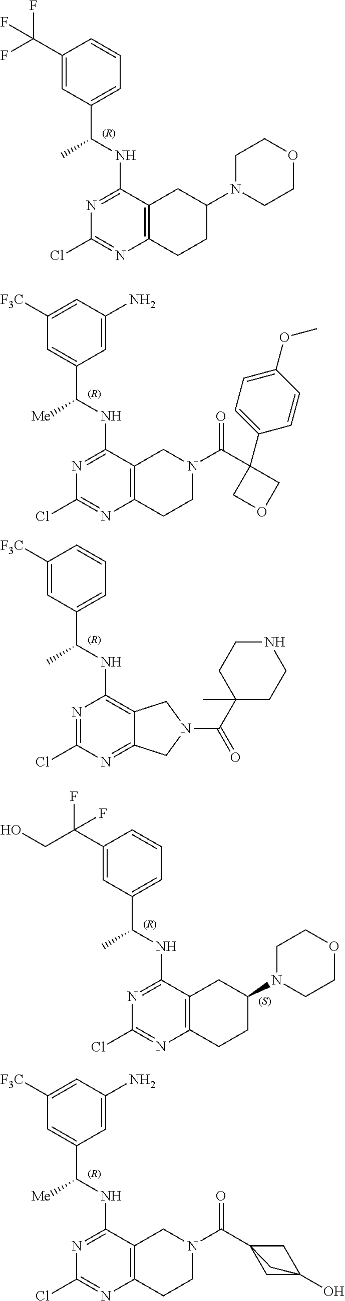

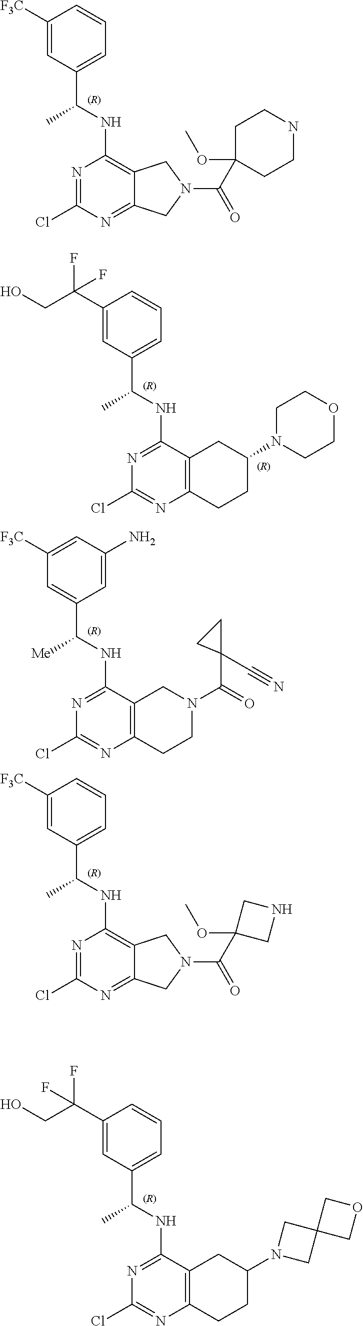

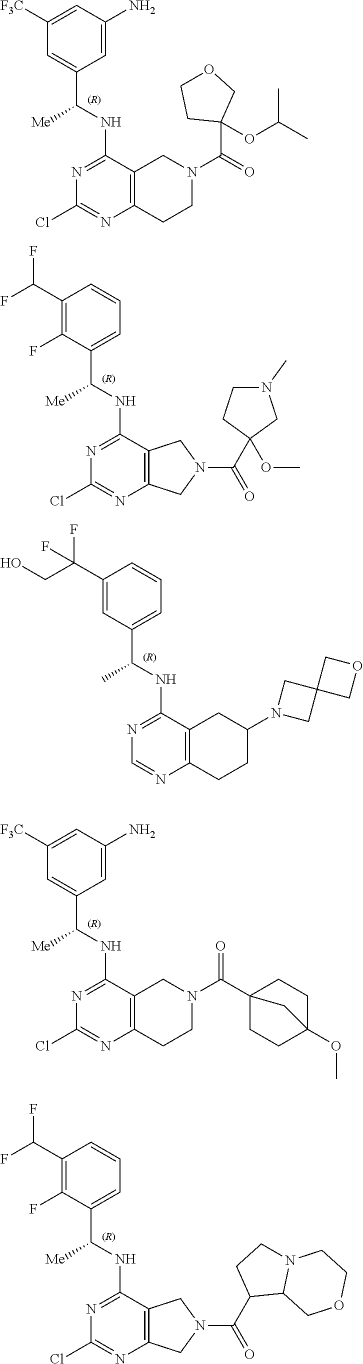

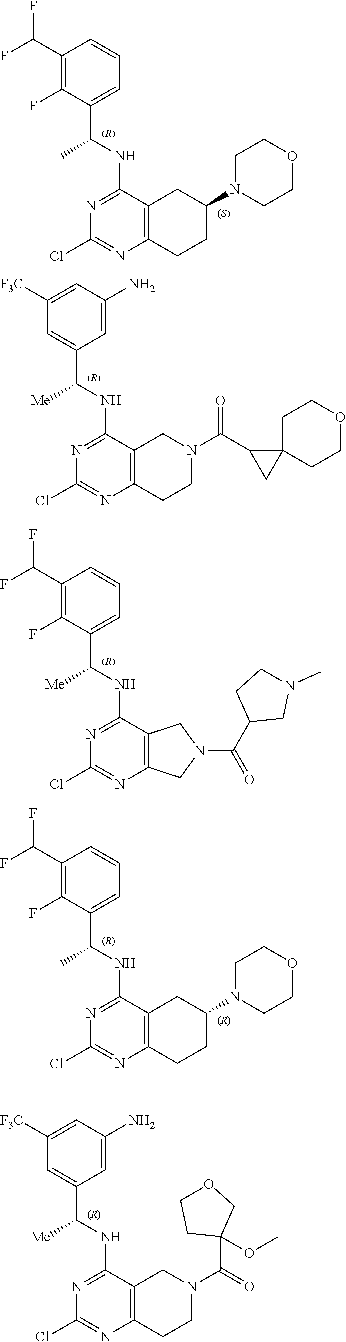

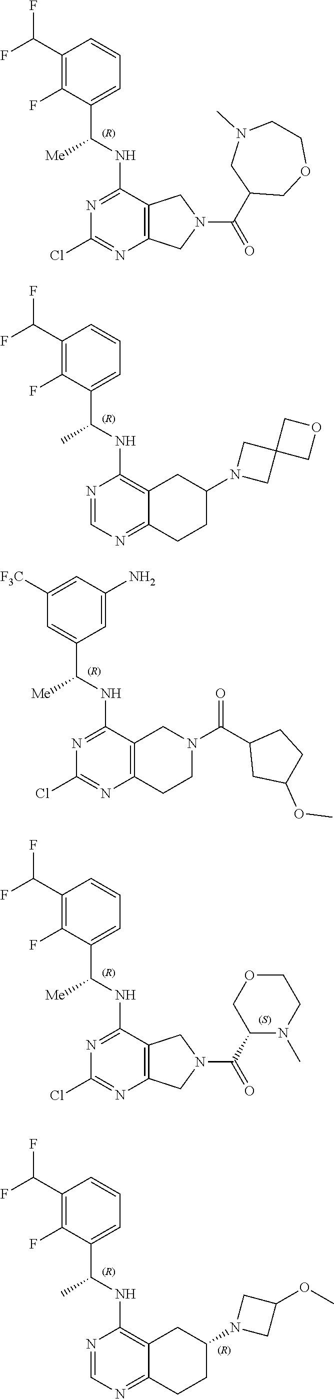

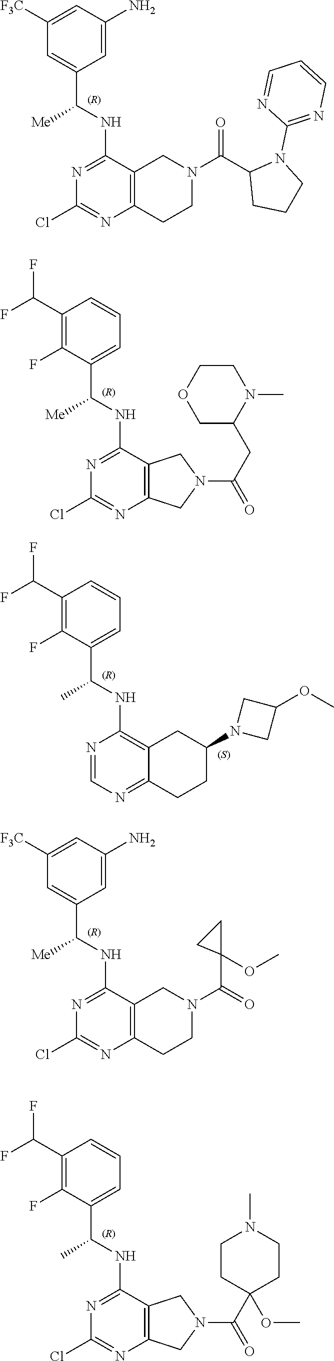

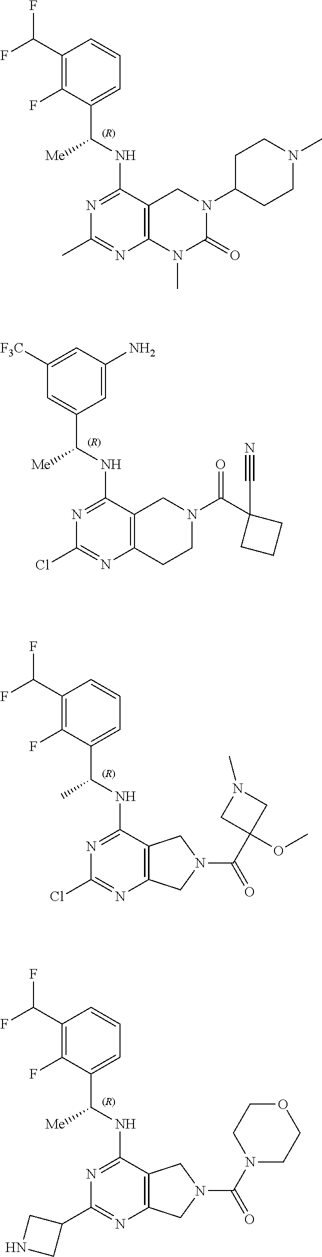

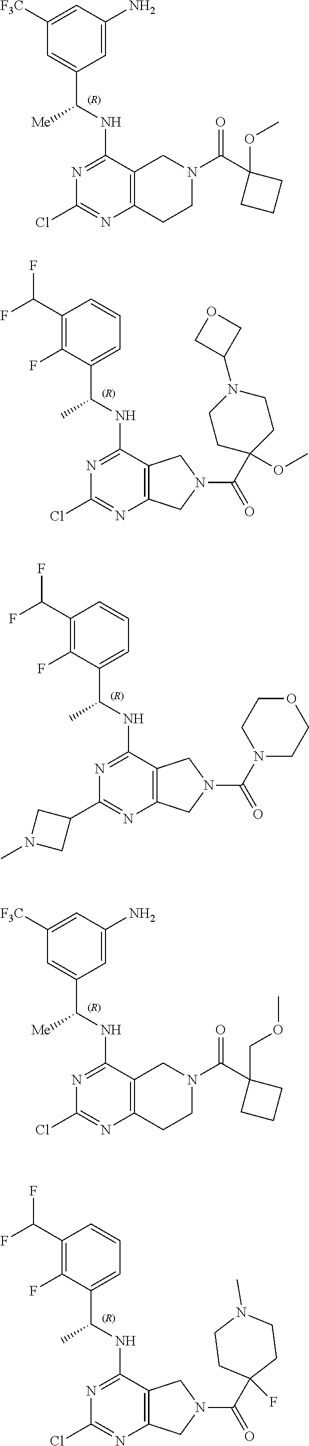

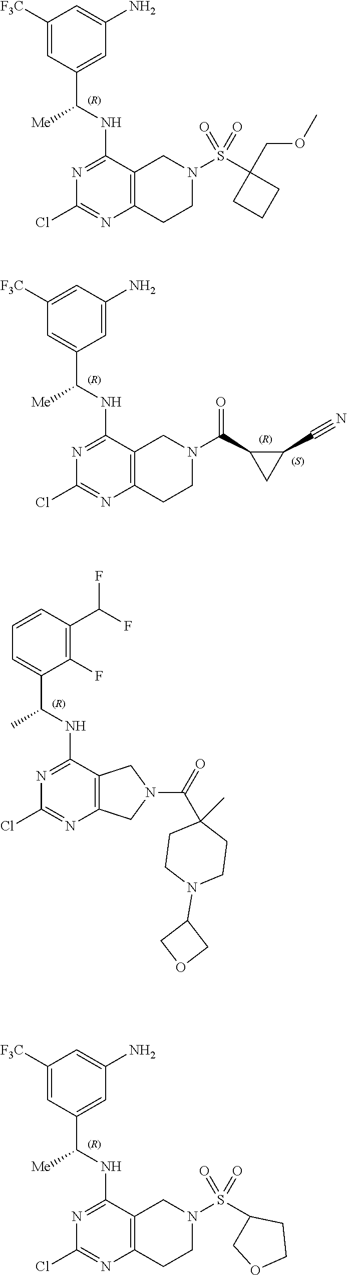

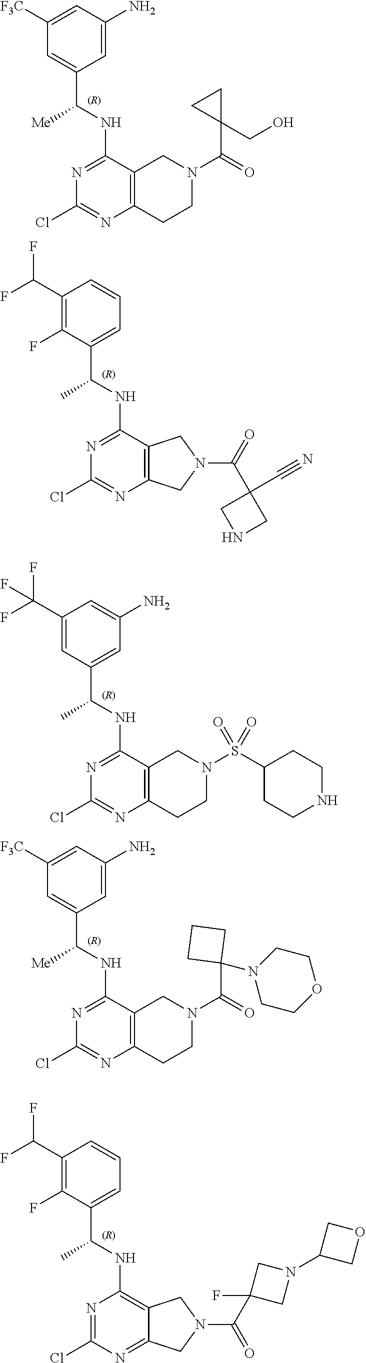

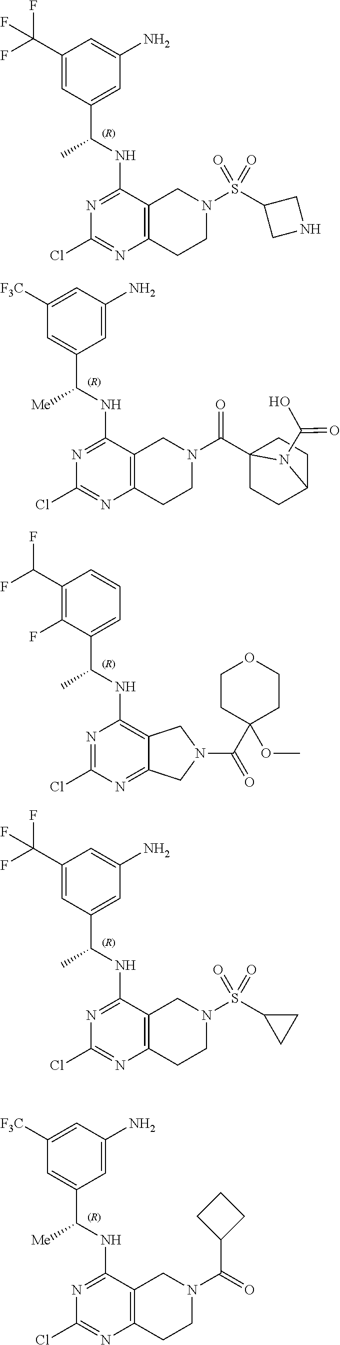

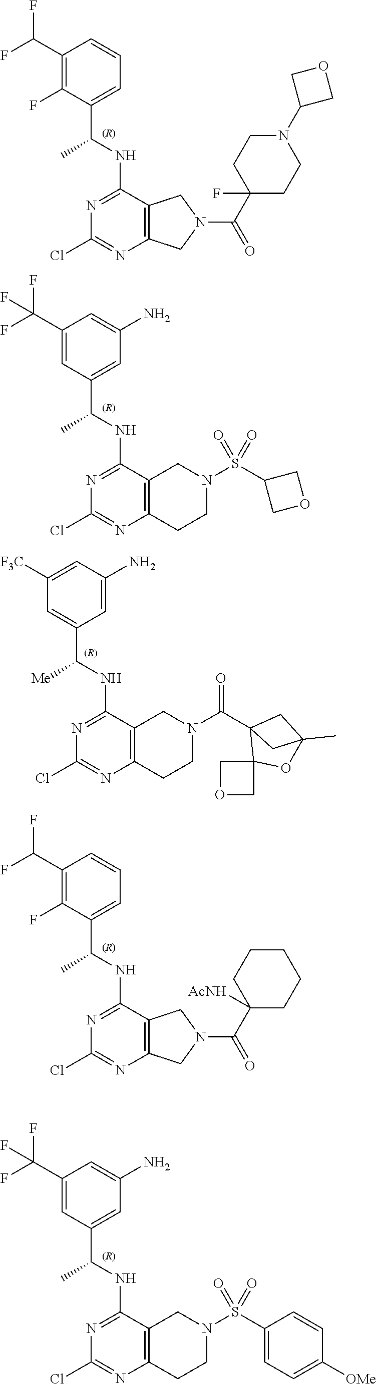

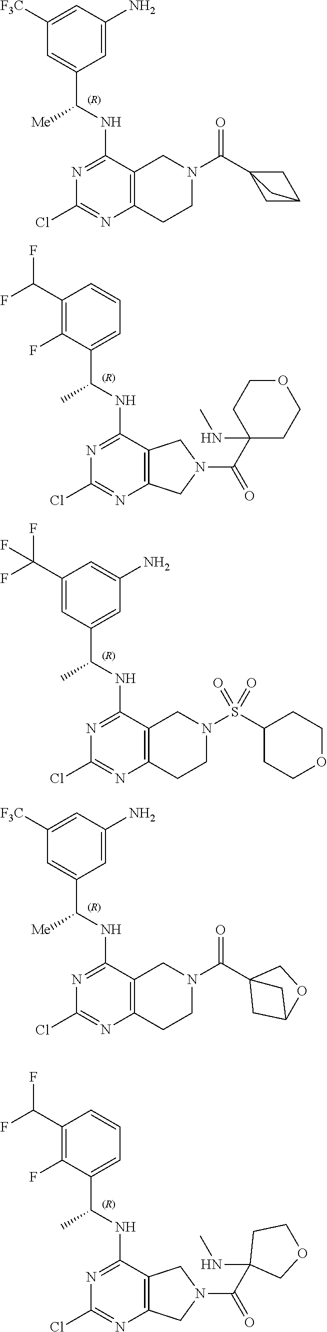

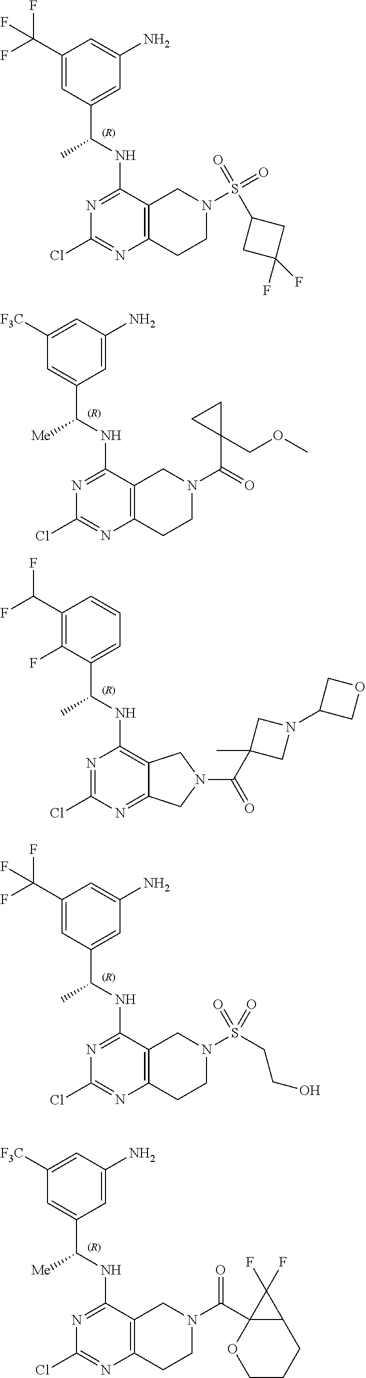

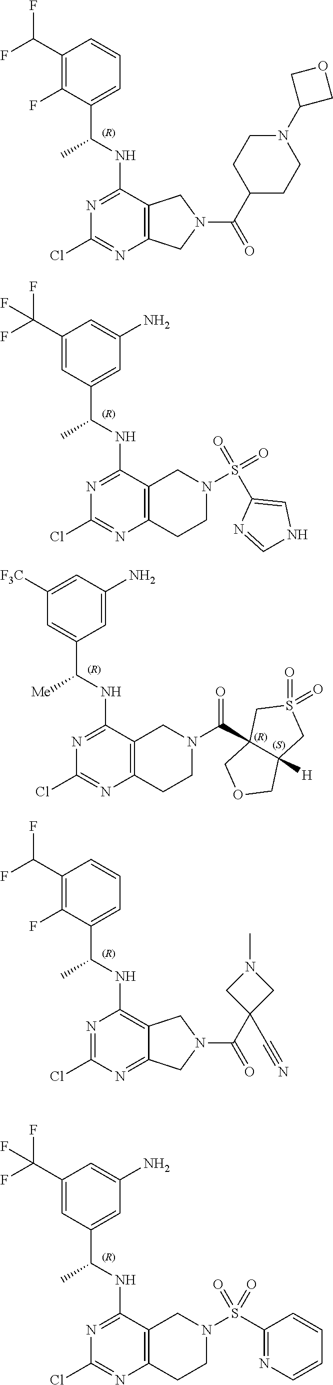

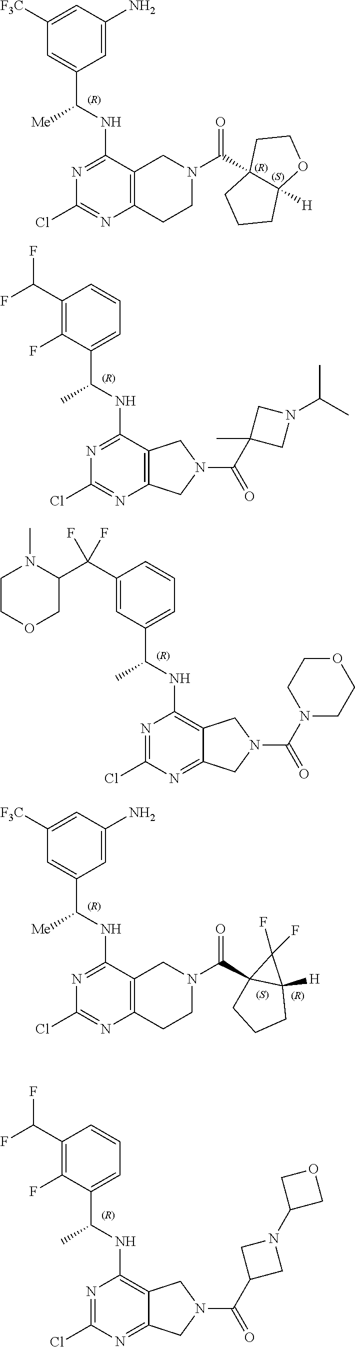

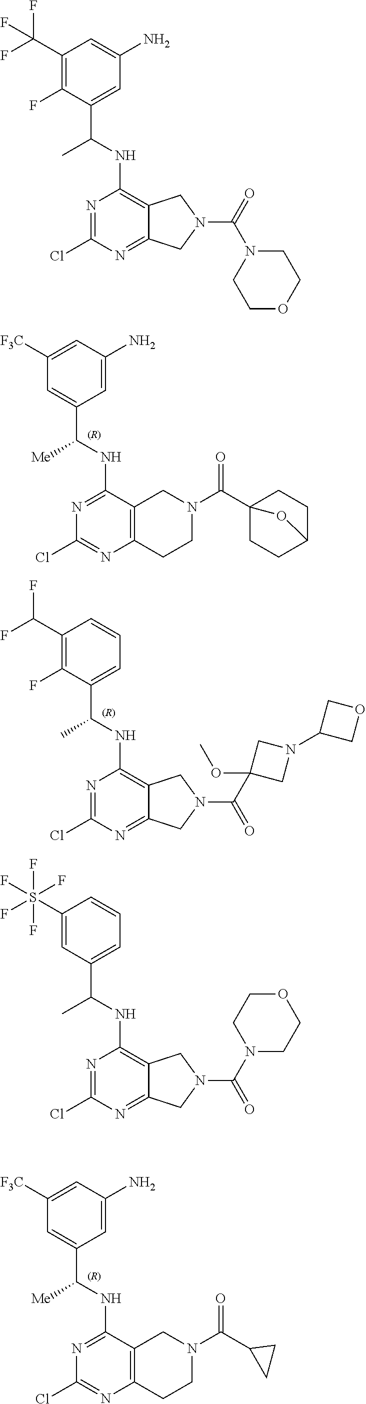

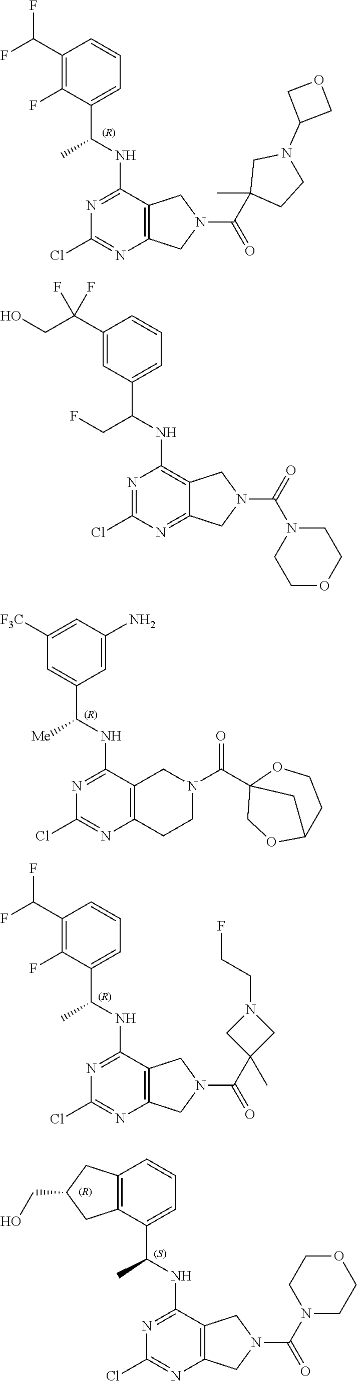

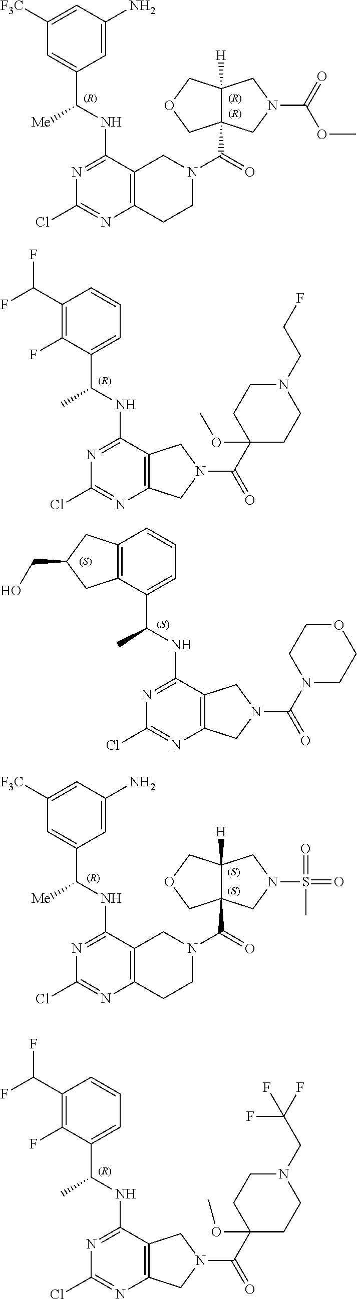

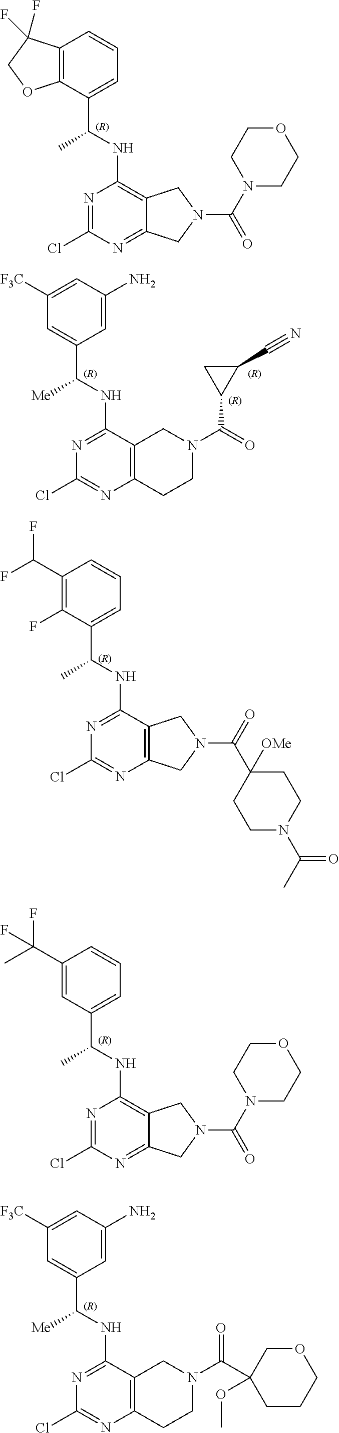

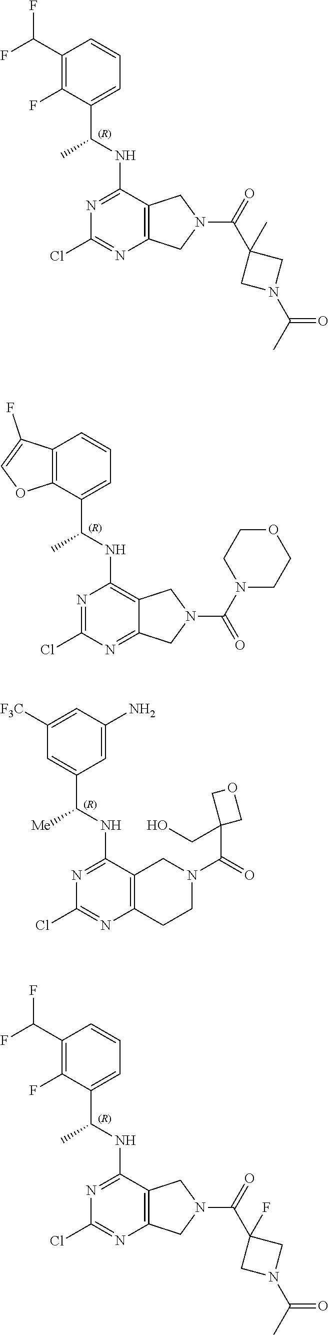

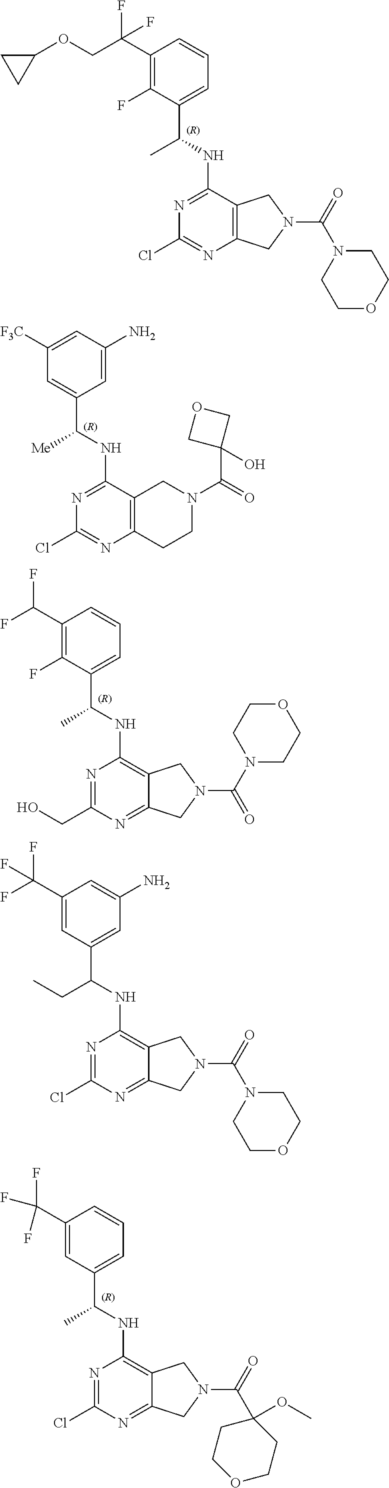

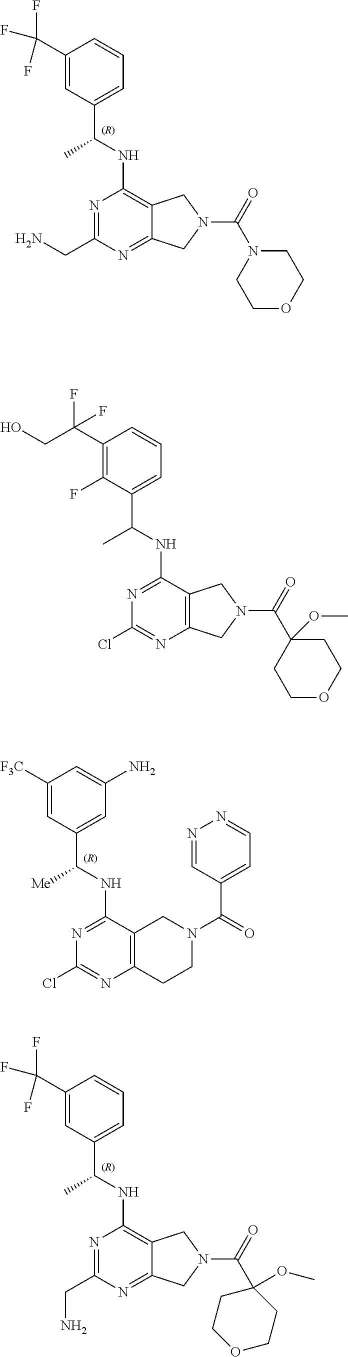

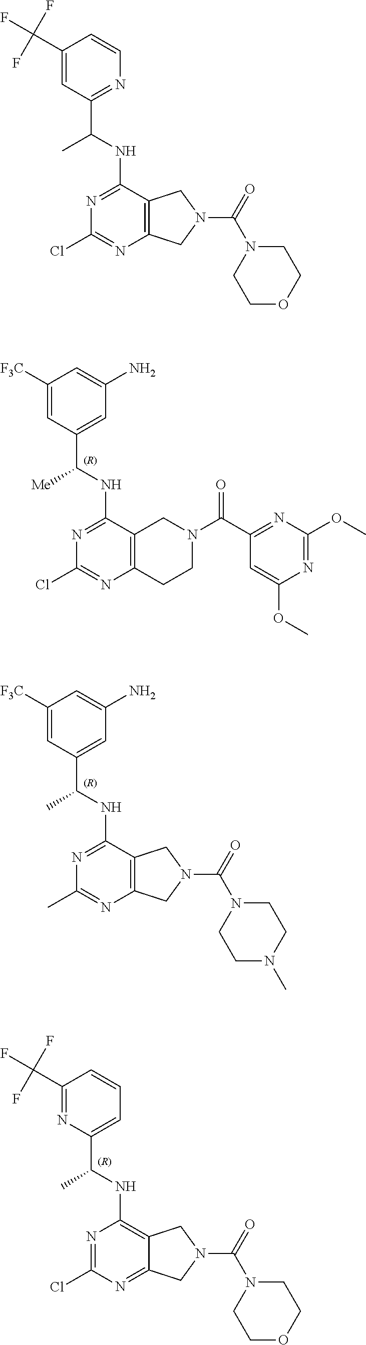

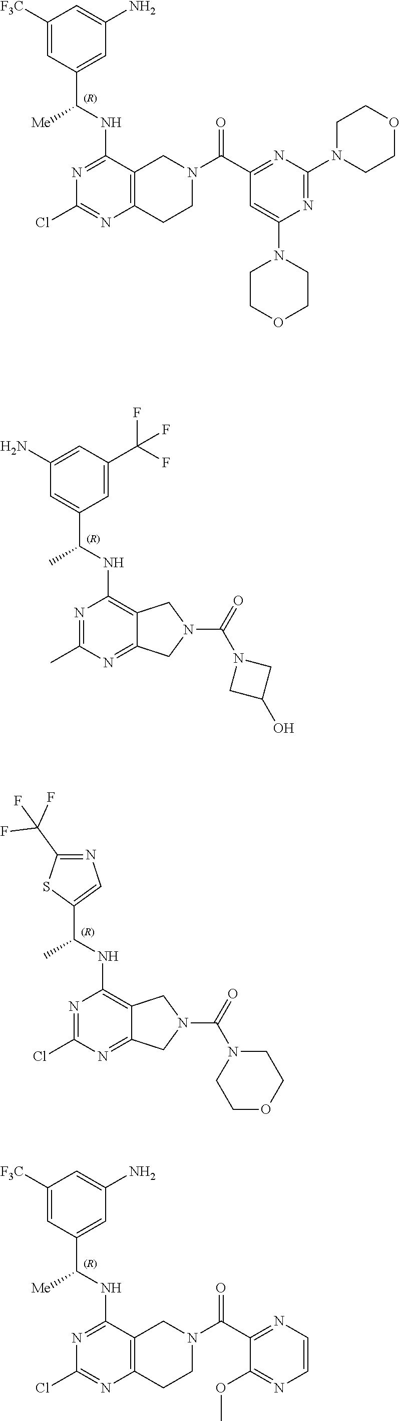

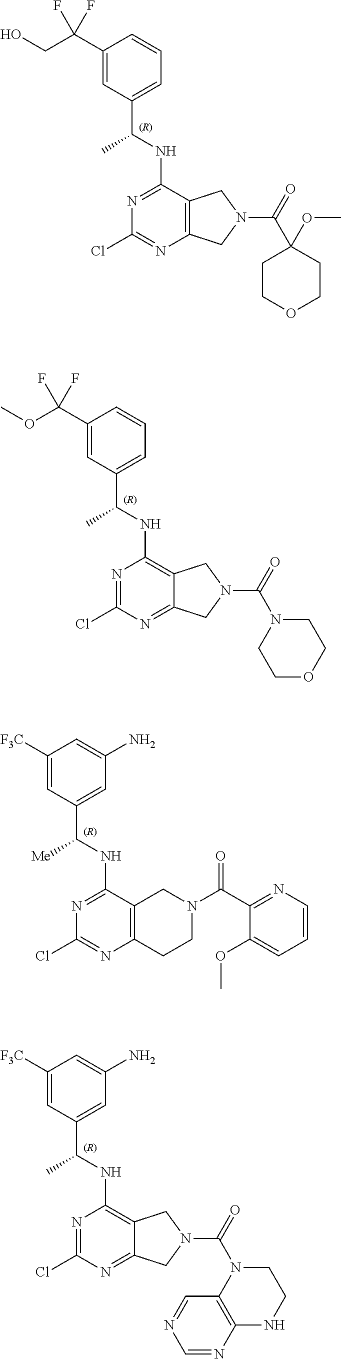

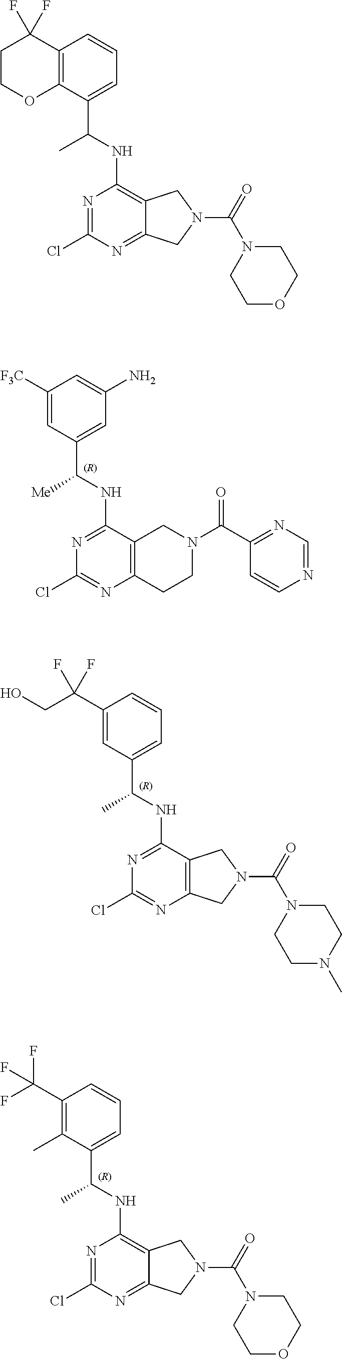

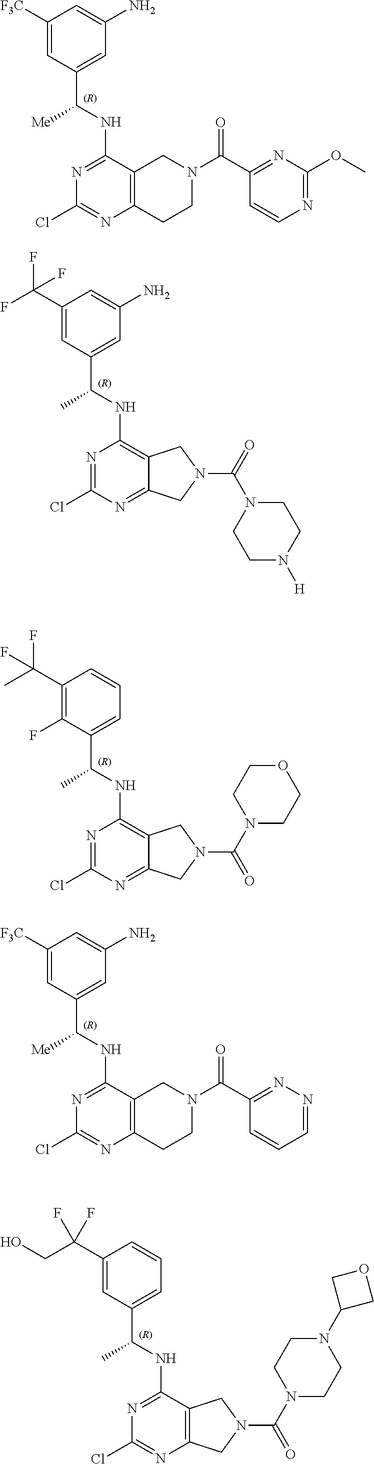

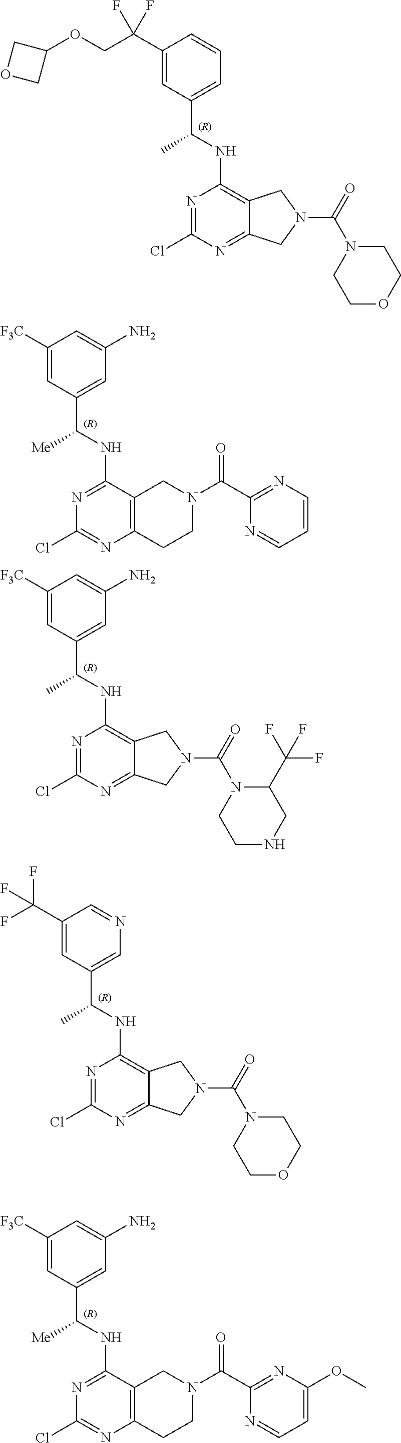

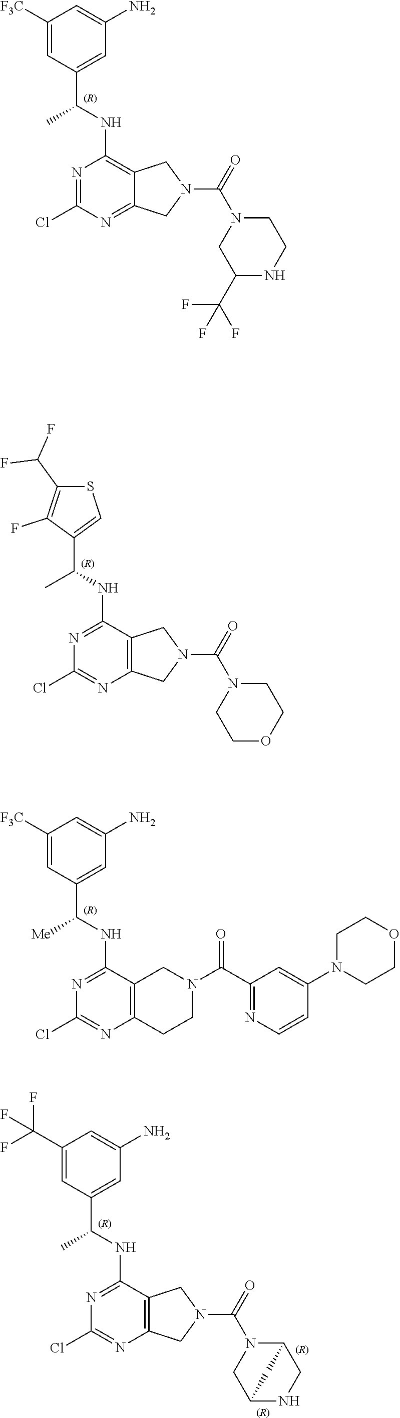

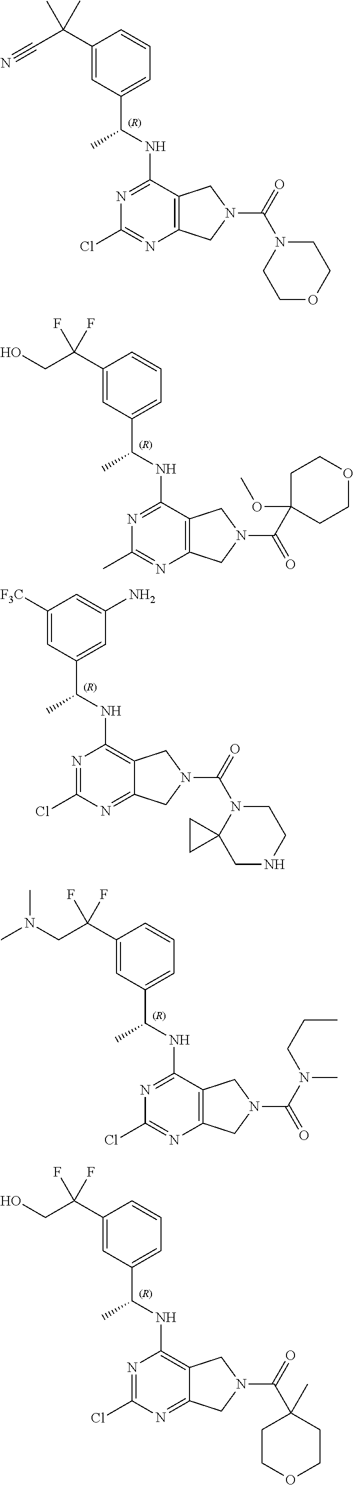

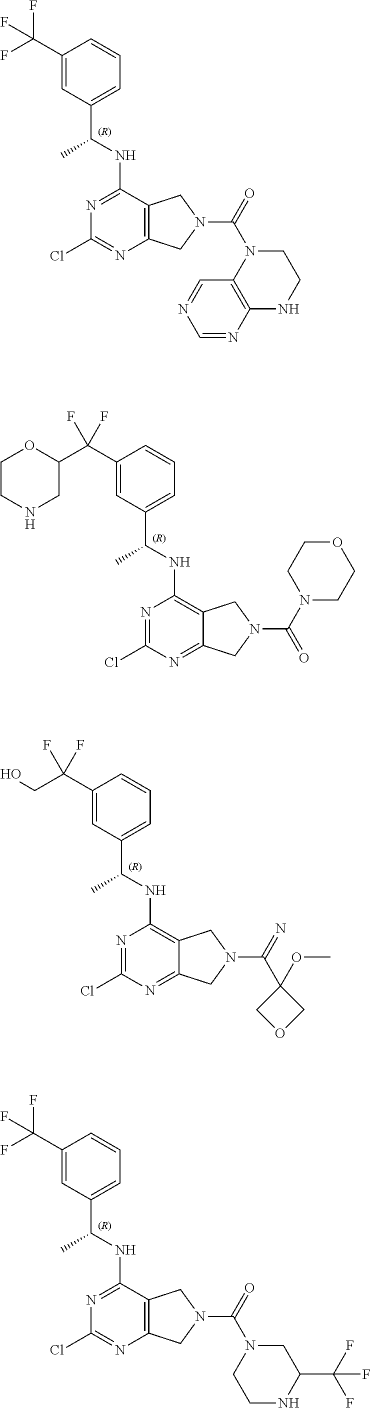

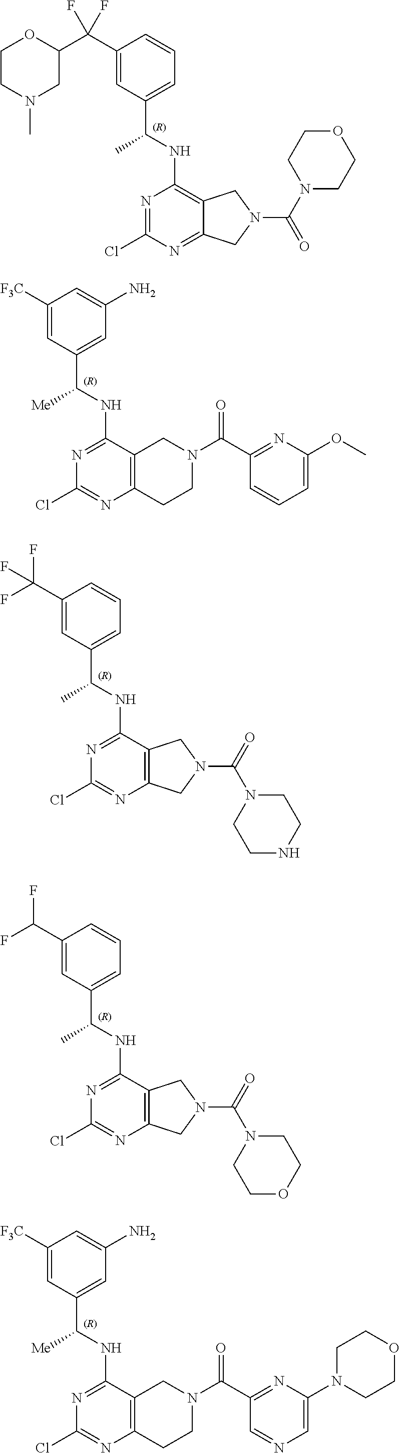

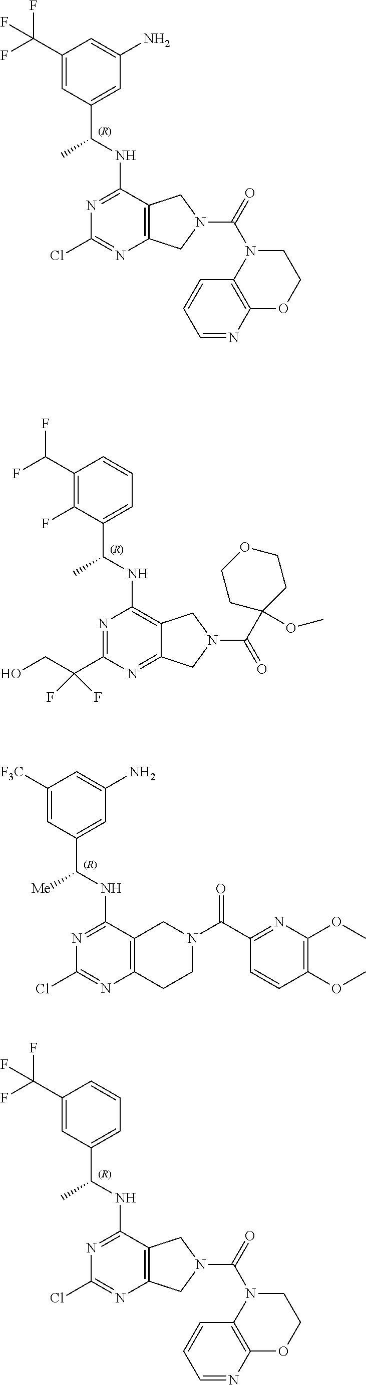

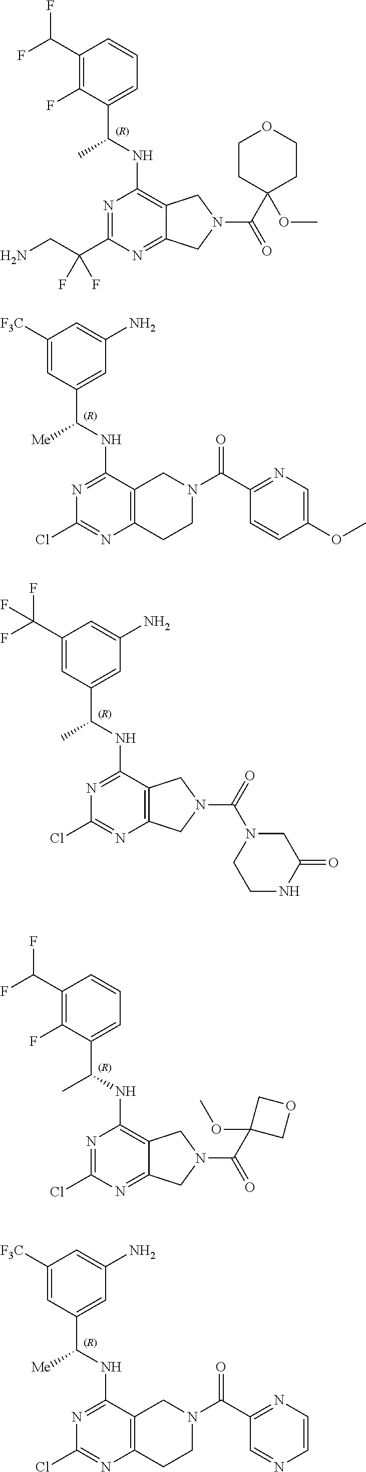

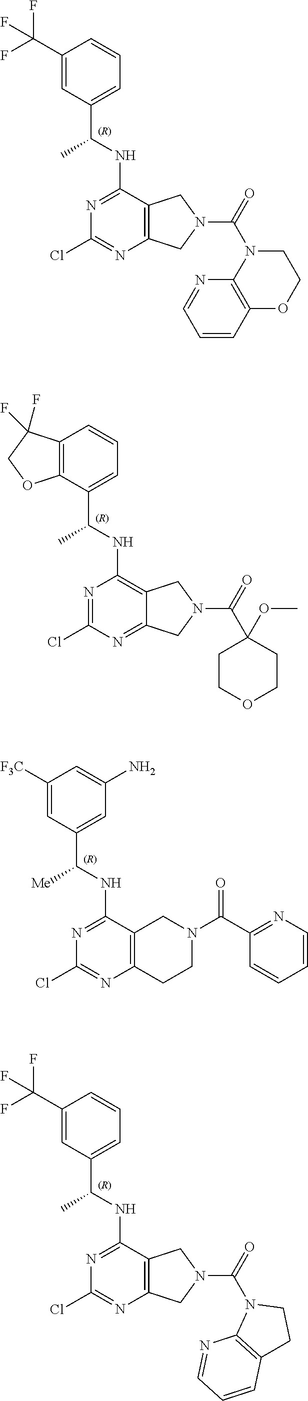

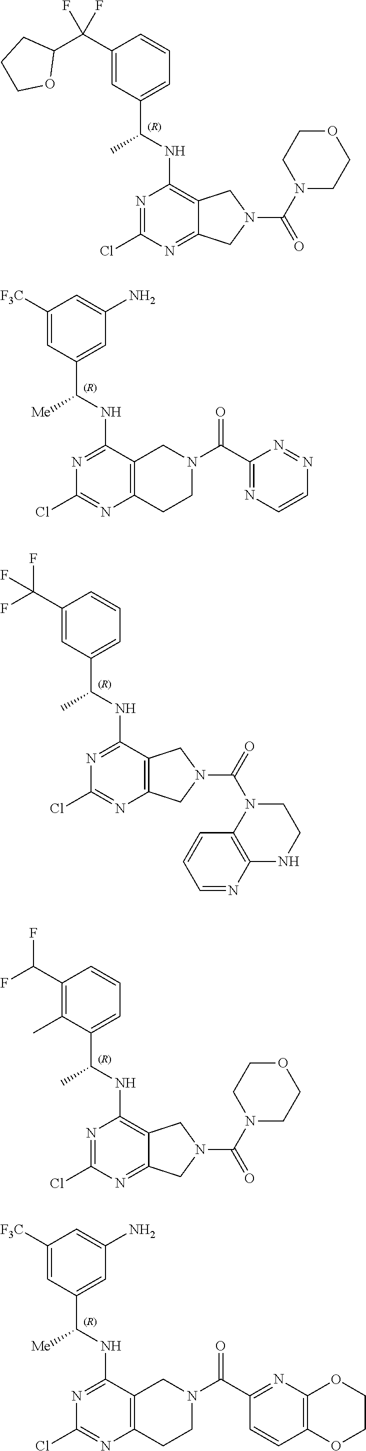

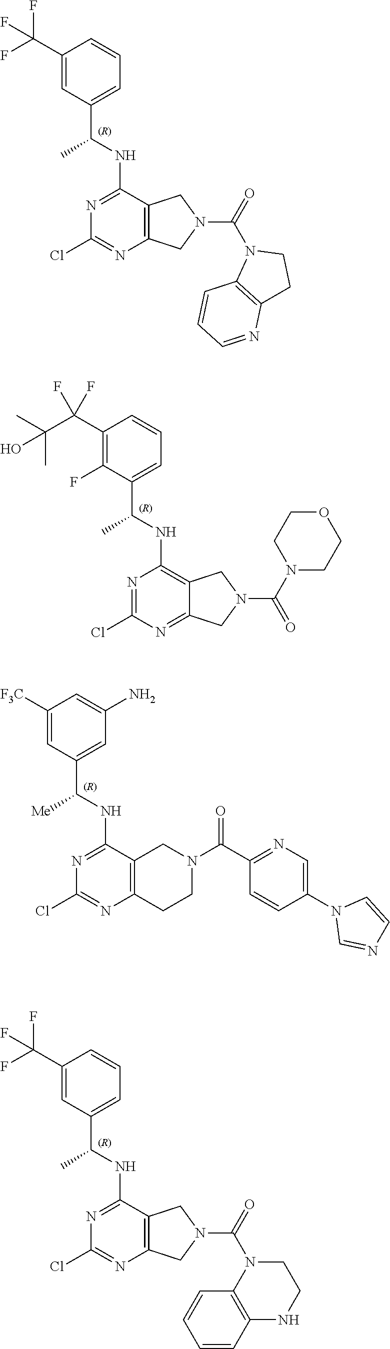

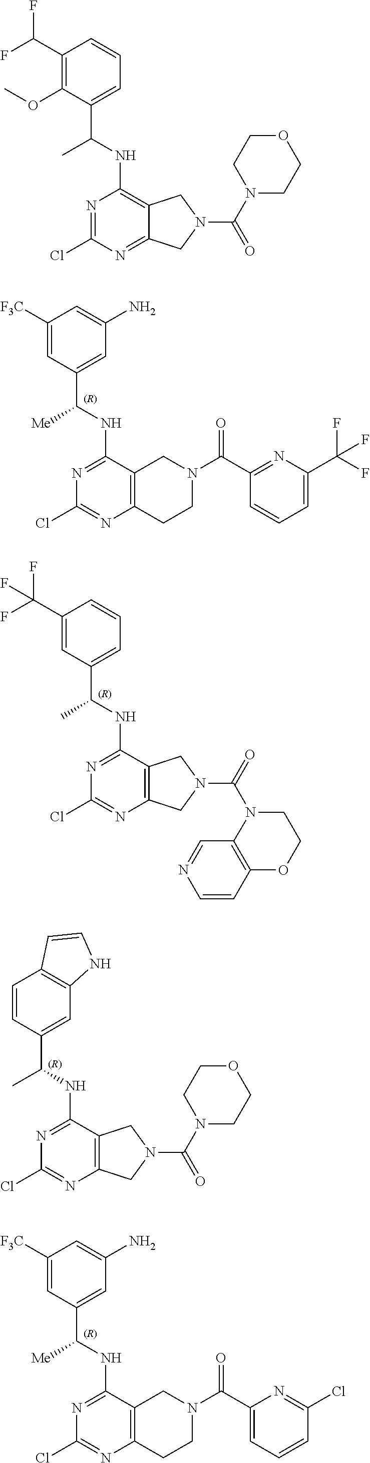

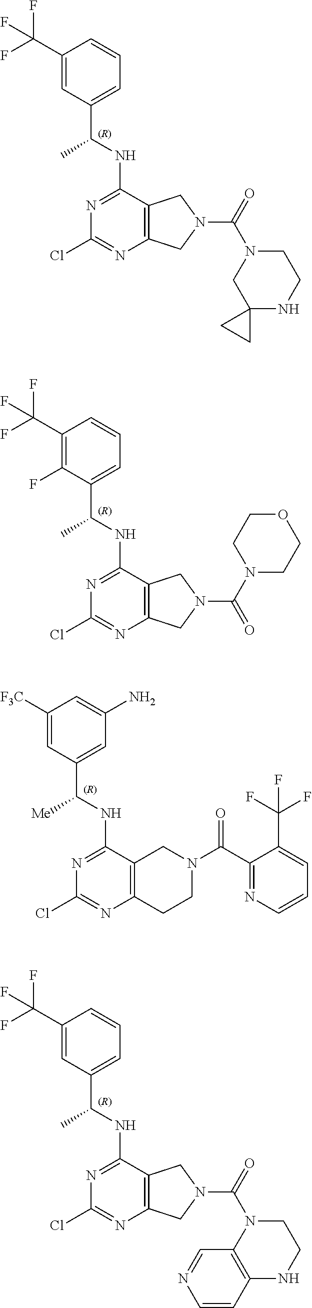

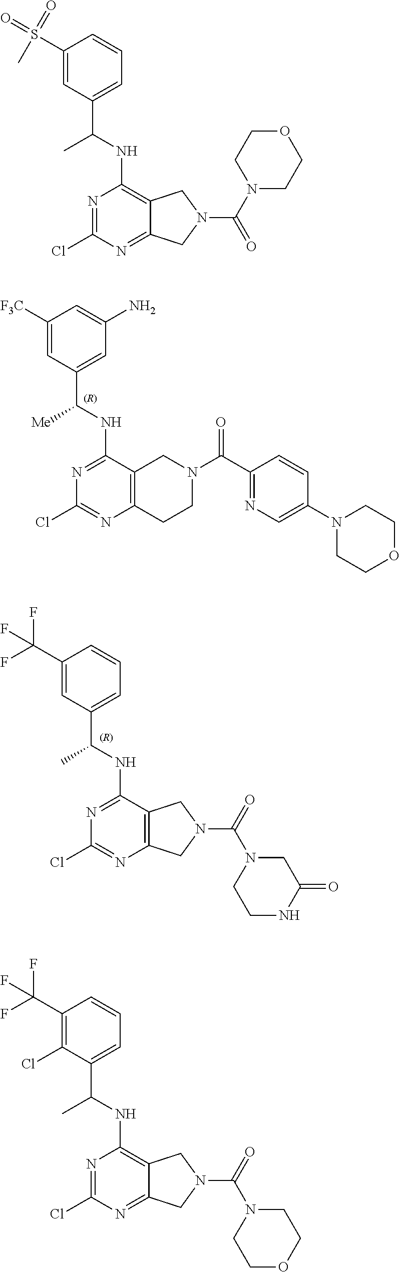

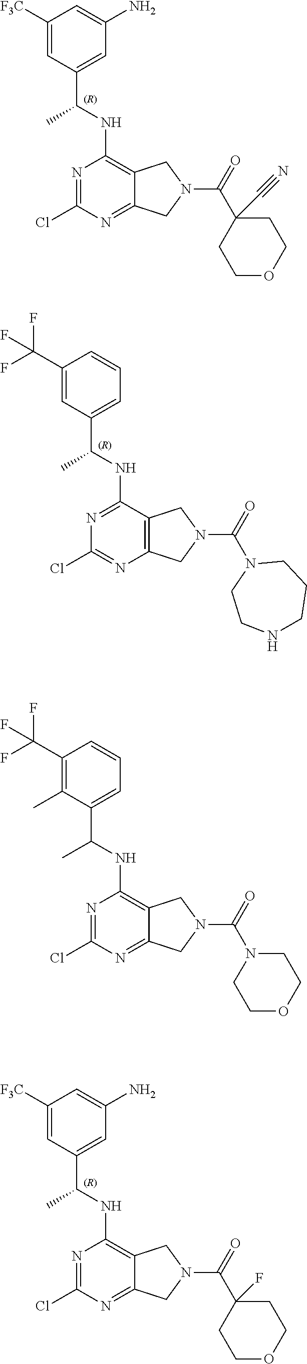

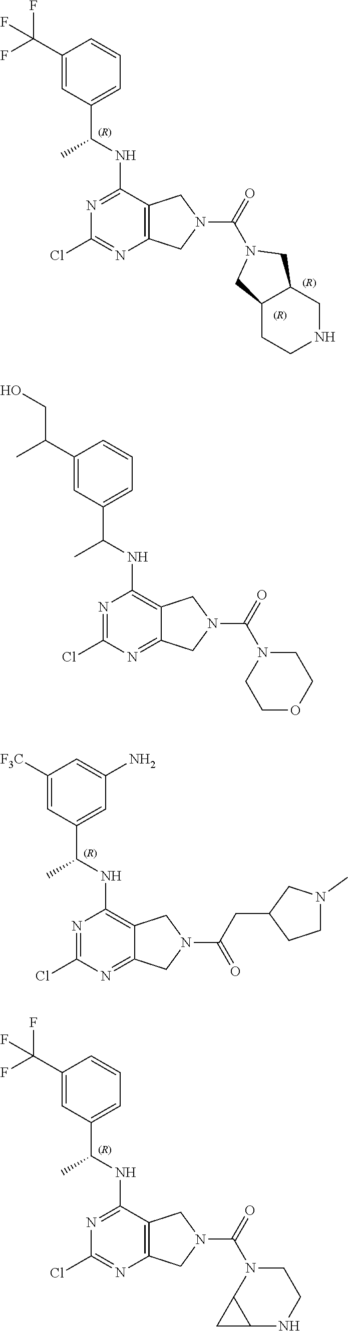

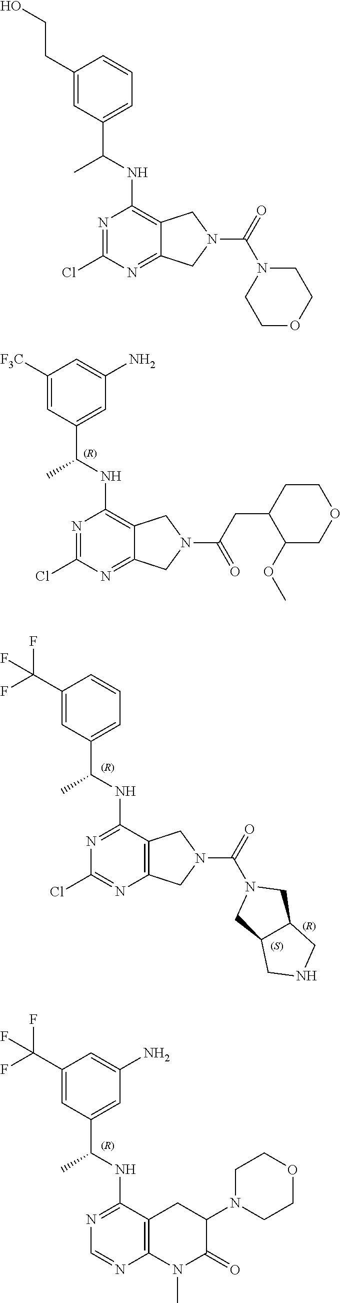

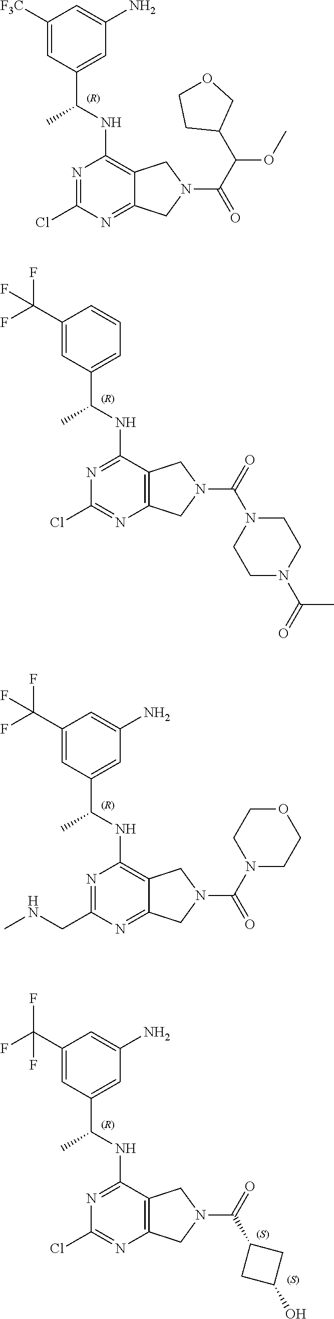

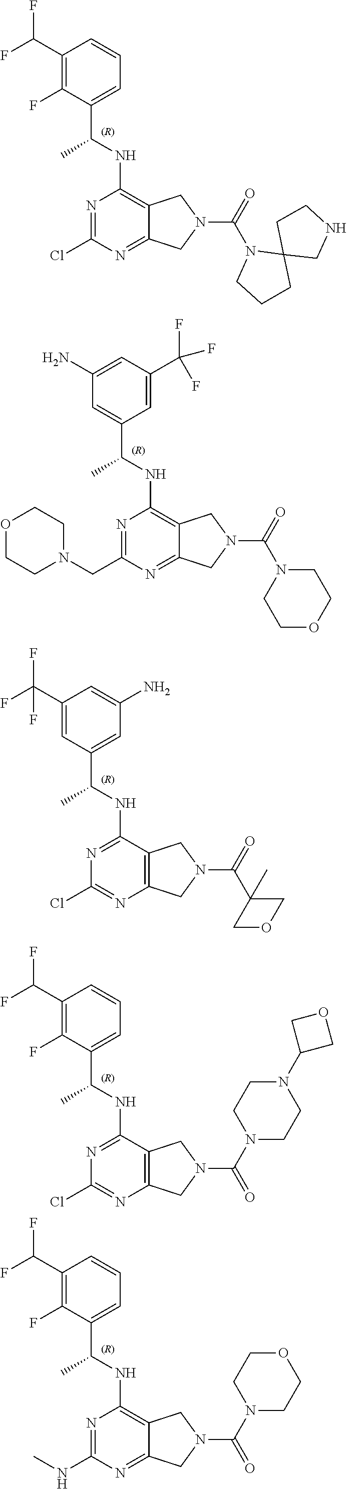

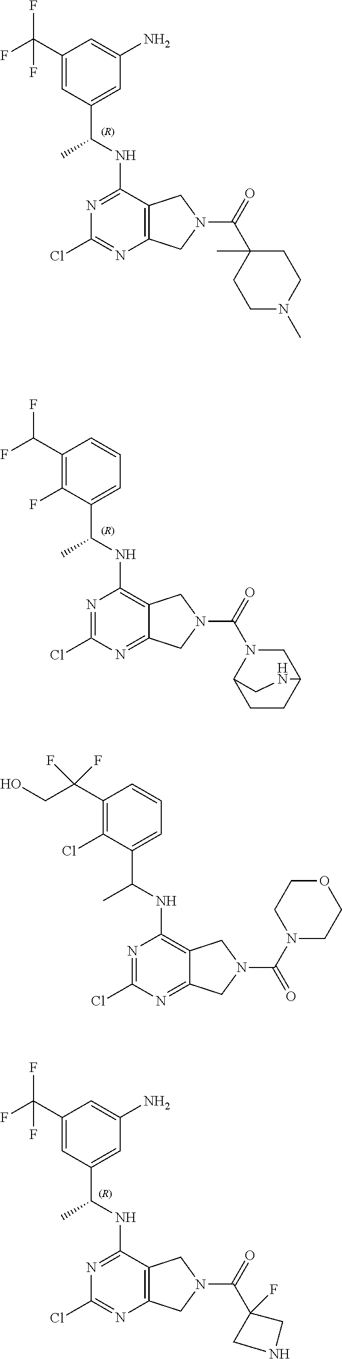

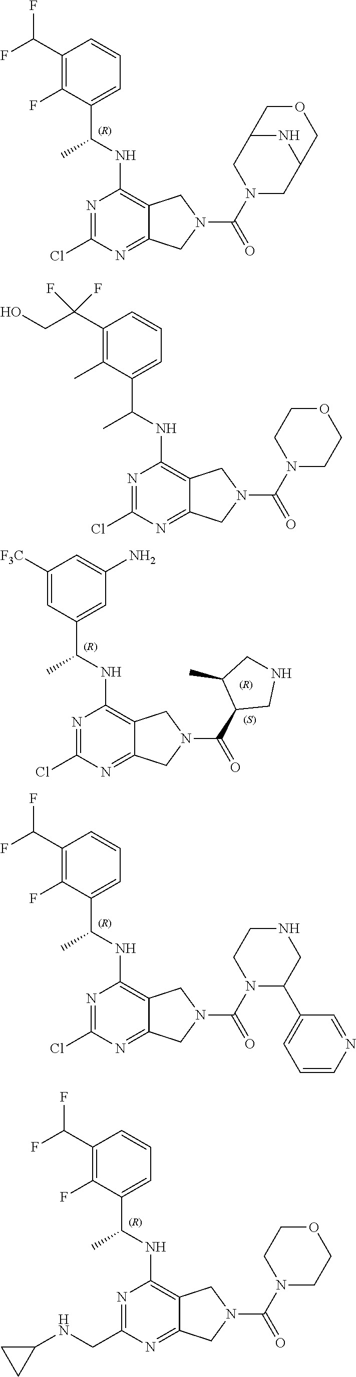

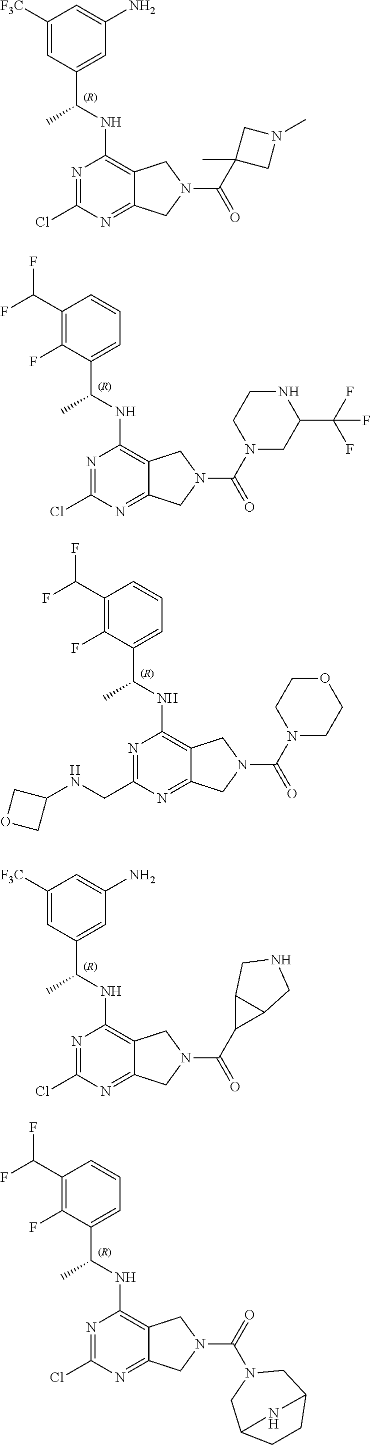

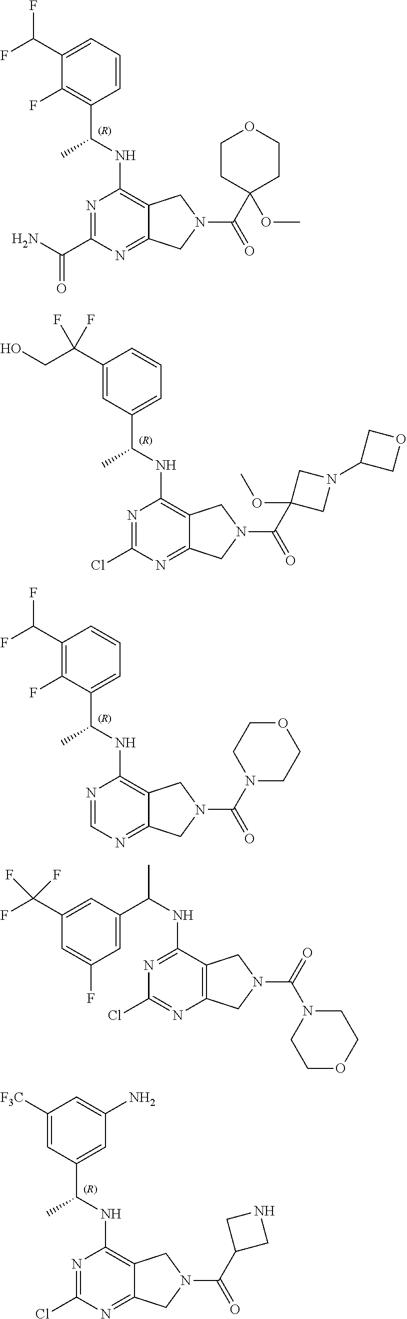

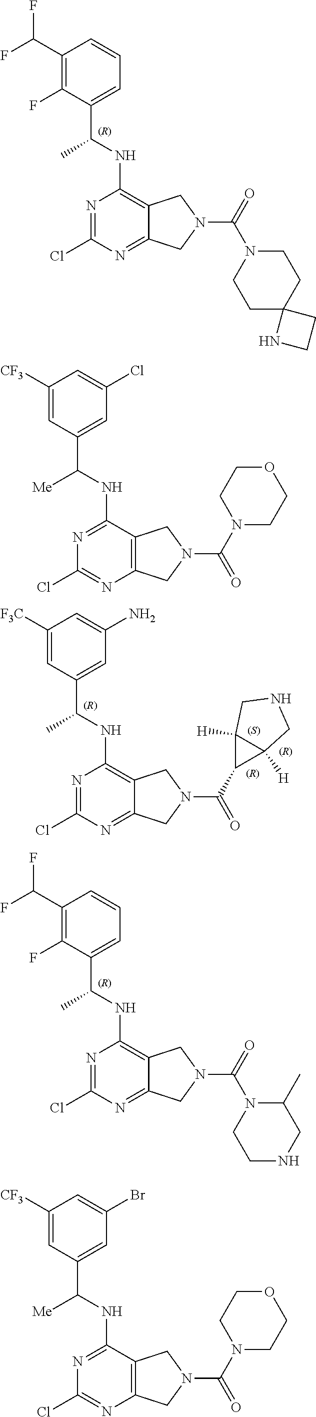

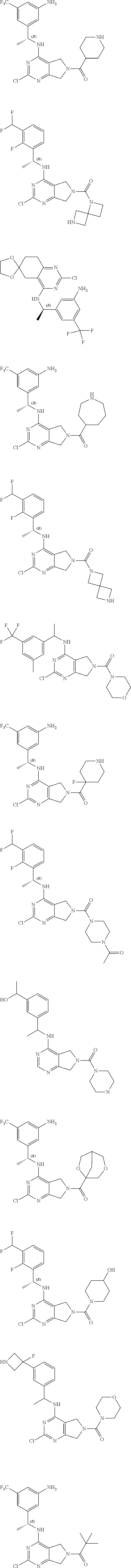

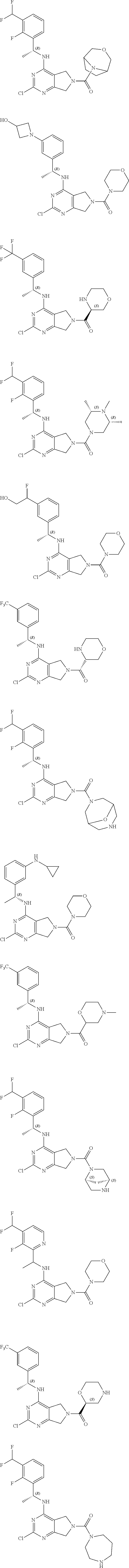

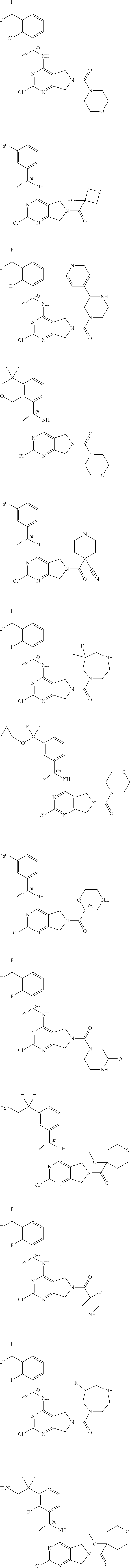

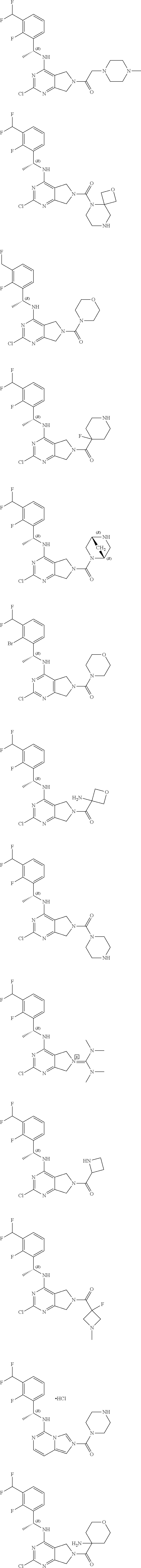

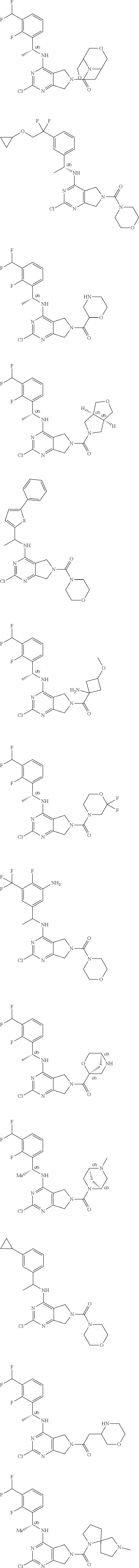

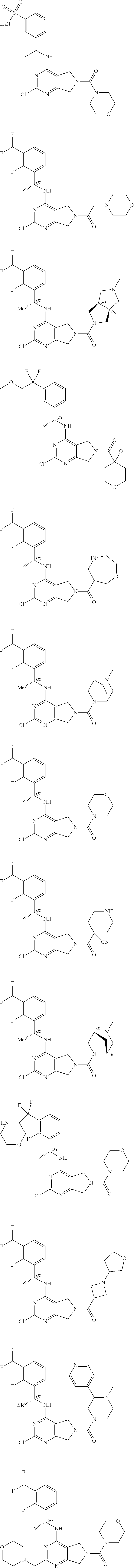

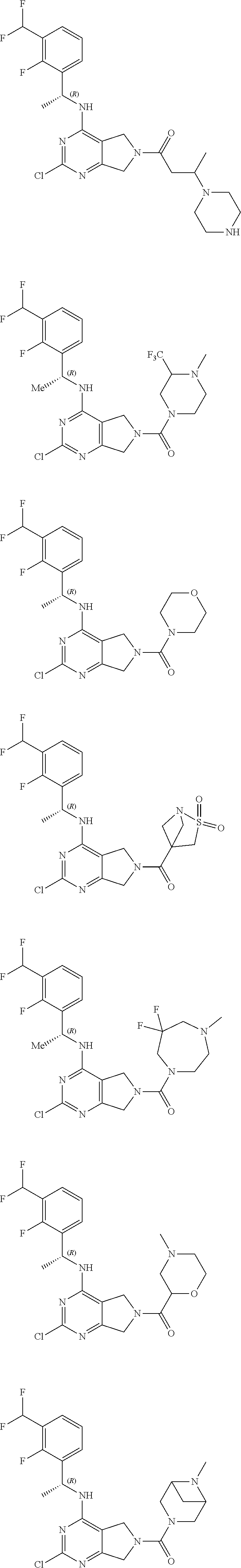

### Methods of Synthesizing the Disclosed Compounds

The compounds of the present invention may be made by a variety of methods, including standard chemistry. Suitable synthetic routes are depicted in the schemes given below.

The compounds of any of the formulae described herein may be prepared by methods known in the art of organic synthesis as set forth in part by the following synthetic schemes and examples. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of their execution, shall be consistent with the preparation of compounds of any formula disclosed herein.

Those skilled in the art will recognize if a stereocenter exists in any of the compounds of the present disclosure. Accordingly, the present invention includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-lnterscience, 1994).

### Preparation of Compounds

The compounds described herein may be made from commercially available starting materials or synthesized using known organic, inorganic, and/or enzymatic processes.

The compounds of the present invention can be prepared in a number of ways well known to those skilled in the art of organic synthesis. By way of example, compounds of the disclosure can be synthesized using the methods described below, together with synthetic methods known in the art of synthetic organic chemistry, or variations thereon as appreciated by those skilled in the art. These methods include but are not limited to those methods described below.

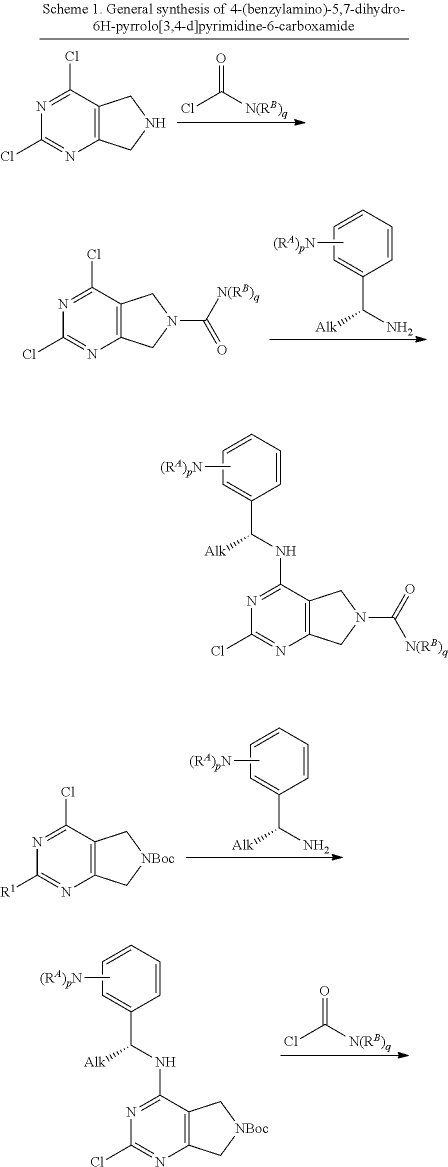

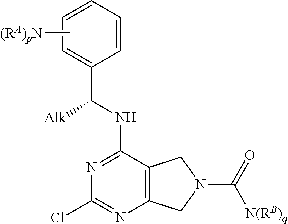

A general synthesis of 4-(benzylamino)-2-chloro-5,7-dihydro-6H-pyrrolo[3,4-d]pyrimidine-6-carboxamides is outlined in Scheme 1. 2,4-dichloro-6,7-dihydro-5H-pyrrolo[3,4-d]pyrimidine or analogous appropriately substituted halogenated heterocyclic ring can be coupled to a substituted carbamic chloride in the presence of an organic base (e.g., DIEA). The resulting urea intermediate can then be coupled to a substituted benzyl amine to give a 5,7-dihydro-6H-pyrrolo[3,4-d]pyrimidine-6-carboxamide. Additional deprotection and/or functionalization steps can be required to produce the final compound.

Alternatively, appropriately protected 4-(benzylamino)-2-alkyl-5,7-dihydro-6H-pyrrolo[3,4-d]pyrimidine-6-carboxamides can be synthesized from 2-chloro-4-alkyl-6,7-dihydro-5H-pyrrolo[3,4-d]pyrimidine or analogous appropriately substituted halogenated heterocyclic ring that is coupled to a benzyl amine in the presence of base followed by deprotection. Subsequent coupling to an appropriately substituted carbamic chloride in the presence of an organic base (e.g., DIEA) results in formation of a urea. Additional deprotection and/or functionalization steps can be required to produce the final compound.

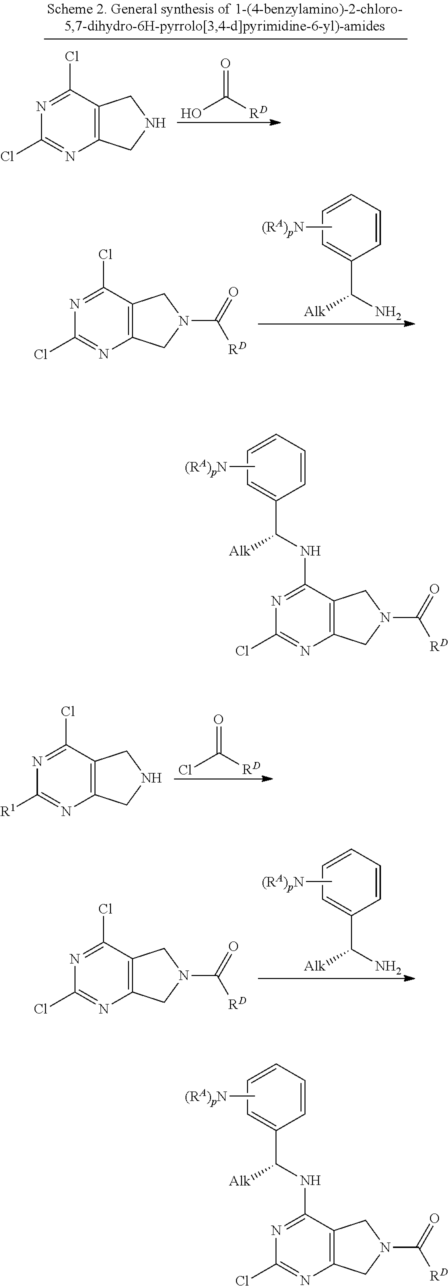

A general synthesis of 1-(4-(benzylamino)-2-chloro-5,7-dihydro-6H-pyrrolo[3,4-d]pyrimidin-6-yl)-amides is outlined in Scheme 2. 2,4-dichloro-6,7-dihydro-5H-pyrrolo[3,4-d]pyrimidine or analogous appropriately substituted halogenated heterocyclic ring can be coupled to a substituted carboxylic acid in the presence of a coupling agents (e.g., T3P). The resulting amide intermediate can then be coupled to a substituted benzyl amine to give a 5,7-dihydro-6H-pyrrolo[3,4-d]pyrimidine-6-yl-amide. Additional deprotection and/or functionalization steps can be required to produce the final compound.

Alternatively, 1-(4-(benzylamino)-2-chloro-5,7-dihydro-6H-pyrrolo[3,4-d]pyrimidin-6-yl)-amides can be synthesized from as 2,4-dichloro-6,7-dihydro-5H-pyrrolo[3,4-d]pyrimidine or analogous appropriately substituted halogenated heterocyclic ring coupled to a substituted carboxylic acid chloride in the presence of an organic base (e.g., DIEA). The resulting amide intermediate can then be coupled to a substituted benzyl amine to give a 5,7-dihydro-6H-pyrrolo[3,4-d]pyrimidine-6-yl-amide. Additional deprotection and/or functionalization steps can be required to produce the final compound.

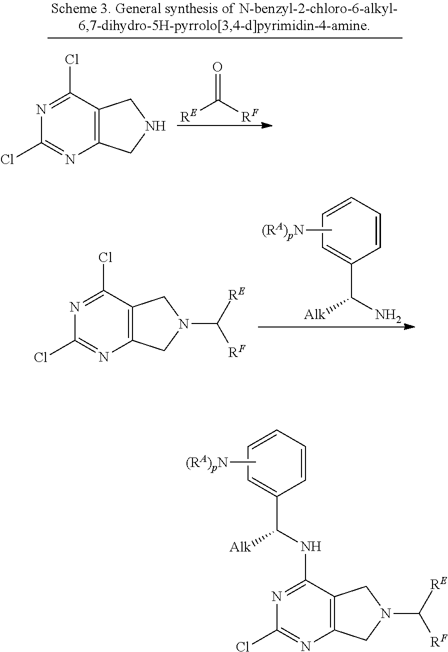

A general synthesis of N-benzyl-2-chloro-6-alkyl-6,7-dihydro-5H-pyrrolo[3,4-d]pyrimidin-4-amines is outlined in Scheme 3. 2,4-dichloro-6,7-dihydro-5H-pyrrolo[3,4-d]pyrimidine or analogous appropriately substituted halogenated heterocyclic ring can be coupled to a substituted ketone in the presence of a reducing agent (e.g., NaBH(OAc)₃). The resulting alkyl intermediate can then be coupled to a substituted benzyl amine to give a N-benzyl-6-alkyl-6,7-dihydro-5H-pyrrolo[3,4-d]pyrimidin-4-amine. Additional deprotection and/or functionalization steps can be required to produce the final compound.

The present disclosure provides a compound of Formula Int-I:

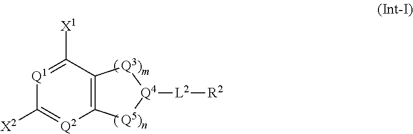

and salts, prodrugs, solvates, hydrates, tautomers, and isomers thereof, wherein:

X¹ is F, Cl, Br, or I;

X² is F, Cl, Br, or I.

Q¹ and Q² are independently CH or N;

each Q³ and Q⁵ are independently C(R^{QC})₂, NR^{QN}, CO, O, or SO₂, wherein each R^{QC} is independently H, F, Cl, Br, or aryl, and wherein each R^{QN} is independently H, C₁₋₆ alkyl, or aryl;

Q⁴ is CH or N;

wherein at least one of Q¹, Q², Q², Q³, Q⁴, and Q⁵ is N, NR^{QN}, O, or SO₂;

m is 0, 1, 2, or 3;

n is 0, 1, 2, or 3;

wherein when m is 0, then n is not 0;

L² is a bond, —C(O)—, —C(O)O—, —C(O)NH(CH₂)ₒ—, —S(O)₂—, —C(O)(CH₂)ₚ—, —(CH₂)ₚ—, or —O—; wherein o is 0, 1, or 2; and wherein p is a number from 1 to 6; R² is H, —(CH₂)_{q}CH₃, cycloalkyl, cycloalkenyl, heterocyclyl, aryl, heteroaryl; wherein q is a number from 1 to 5; wherein each cycloalkyl, cycloalkenyl, heterocyclyl, aryl, or heteroaryl is optionally substituted with one or more C₁₋₆ alkyl, —OH, halogen, —C(O)R^{2a}, or —C(O)NR^{2b}R^{2c}; wherein R^{2a} is C₁₋₆ alkyl or —(CH₂)ᵣOCH₃, wherein r is 1, 2, or 3; wherein R^{2b} is H or C₁₋₆ alkyl; and wherein R^{2c} is H or C₁₋₆ alkyl.

The present disclosure provides a compound of Formula Int-Ia:

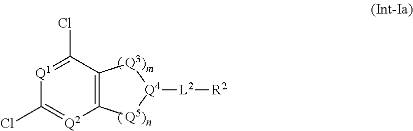

and salts, prodrugs, solvates, hydrates, tautomers, and isomers thereof, wherein:

Q¹ and Q² are independently CH or N;

each Q³ and Q⁵ are independently C(R^{QC})₂, NR^{QN}, CO, O, or SO₂, wherein each R^{QC} is independently H, F, Cl, Br, or aryl, and wherein each R^{QN} is independently H, C₁₋₆ alkyl, or aryl;

Q⁴ is CH or N;

wherein at least one of Q¹, Q², Q², Q³, Q⁴, and Q⁵ is N, NR^{QN}, O, or SO₂;

m is 0, 1, 2, or 3;

n is 0, 1, 2, or 3;

wherein when m is 0, then n is not 0;

L² is a bond, —C(O)—, —C(O)O—, —C(O)NH(CH₂)ₒ—, —S(O)₂—, —C(O)(CH₂)ₚ—, —(CH₂)ₚ—, or —O—; wherein o is 0, 1, or 2; and wherein p is a number from 1 to 6;

R² is H, —(CH₂)_{q}CH₃, cycloalkyl, cycloalkenyl, heterocyclyl, aryl, heteroaryl; wherein q is a number from 1 to 5; wherein each cycloalkyl, cycloalkenyl, heterocyclyl, aryl, or heteroaryl is optionally substituted with one or more C₁₋₆ alkyl, —OH, halogen, —C(O)R^{2a}, or —C(O)NR_{2b}R_{2c}; wherein R^{2a} is C₆ alkyl or —(CH₂)ᵣOCH₃.

The present disclosure provides a compound, and salts, solvates, stereoisomers, and tautomers thereof, selected from the group consisting of:

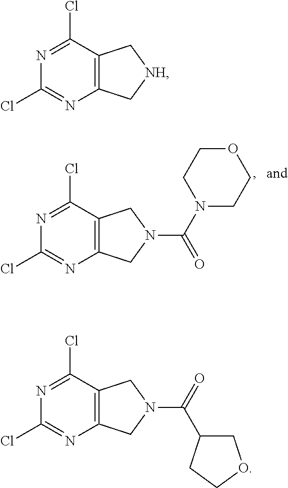 [SMILES: Clc1nc(Cl)c2c(n1)CNC2 ; O=C(N1CCOCC1)N1Cc2nc(Cl)nc(Cl)c2C1 ; O=C(C1CCOC1)N1Cc2nc(Cl)nc(Cl)c2C1]

The present disclosure provides a compound, and salts, solvates, stereoisomers, and tautomers thereof, having the formula:

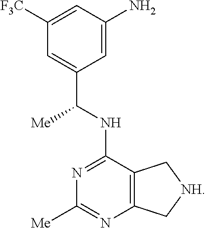 [SMILES: Cc1nc2c(c(N[C@H](C)c3cc(N)cc(C(F)(F)F)c3)n1)CNC2]

### Therapeutic Use

Due to their biological properties the compounds of the present disclosure, their tautomers, racemates, enantiomers, diastereomers, mixtures thereof and the salts of all the above-mentioned forms may be suitable for treating diseases characterized by excessive or abnormal cell proliferation such as cancer.

For example, the following cancers, tumors and other proliferative diseases may be treated with compounds of the present disclosure, without being restricted thereto:

cancers/tumors/carcinomas of the head and neck: e.g., tumors/carcinomas/cancers of the nasal cavity, paranasal sinuses, nasopharynx, oral cavity (including lip, gum, alveolar ridge, retromolar trigone, floor of mouth, tongue, hard palate, buccal mucosa), oropharynx (including base of tongue, tonsil, tonsillar pilar, soft palate, tonsillar fossa, pharyngeal wall), middle ear, larynx (including supraglottis, glottis, subglottis, vocal cords), hypopharynx, salivary glands (including minor salivary glands); intraocular cancers (e.g., uveal melanoma), and orbital and adnexal cancers;

cancers/tumors/carcinomas of the lung: e.g., non-small cell lung cancer (NSCLC) (squamous cell carcinoma, spindle cell carcinoma, adenocarcinoma, large cell carcinoma, clear cell carcinoma, bronchioalveolar), small cell lung cancer (SCLC) (oat cell cancer, intermediate cell cancer, combined oat cell cancer);

neoplasms of the mediastinum: e.g., neurogenic tumors (including neurofibroma, neurilemoma, malignant schwannoma, neurosarcoma, ganglioneuroblastoma, ganglioneuroma, neuroblastoma, pheochromocytoma, paraganglioma), germ cell tumors (including seminoma, teratoma, non-seminoma), thymic tumors (including thymoma, thymolipoma, thymic carcinoma, thymic carcinoid), mesenchymal tumors (including fibroma, fibrosarcoma, lipoma, liposarcoma, myxoma, mesothelioma, leiomyoma, leiomyosarcoma, rhabdomyosarcoma, xanthogranuloma, mesenchymoma, hemangioma, hemangioendothelioma, hemangiopericytoma, lymphangioma, lymphangiopericytoma, lymphangiomyoma), astrocytoma (cerebral, cerebellar, diffuse, fibrillary, anaplastic, pilocytic, protoplasmic, gemistocytary), glioblastoma, gliomas, oligodendrogliomas, oligoastrocytomas, ependymomas, ependymoblastomas, choroid plexus tumors, medulloblastomas, meningiomas, schwannomas, hemangioblastomas, hemangiomas, hemangiopericytomas, neuromas, ganglioneuromas, neuroblastomas, retinoblastomas, neurinomas (e.g., acoustic), spinal axis tumors;

cancers/tumors/carcinomas of the gastrointestinal (GI) tract: e.g., tumors/carcinomas/cancers of the esophagus, stomach (gastric cancer), pancreas, liver and biliary tree (including hepatocellular carcinoma (HCC), e.g., childhood HCC, fibrolamellar HCC, combined HCC, spindle cell HCC, clear cell HCC, giant cell HCC, carcinosarcoma HCC, sclerosing HCC; hepatoblastoma; cholangiocarcinoma; cholangiocellular carcinoma; hepatic cystadenocarcinoma; angiosarcoma, hemangioendothelioma, leiomyosarcoma, malignant schwannoma, fibrosarcoma, Klatskin tumor), gall bladder, extrahepatic bile ducts, small intestine (including duodenum, jejunum, ileum), large intestine (including cecum, colon, rectum, anus; colorectal cancer, gastrointestinal stroma tumor (GIST)), genitourinary system (including kidney, e.g., renal pelvis, renal cell carcinoma (RCC), nephroblastoma (Wilms' tumor), hypernephroma, Grawitz tumor; ureter; urinary bladder, e.g., urachal cancer, urothelial cancer; urethra, e.g., distal, bulbomembranous, prostatic; prostate (androgen dependent, androgen independent, castration resistant, hormone independent, hormone refractory), penis);

cancers/tumors/carcinomas of the testis: e.g., seminomas, non-seminomas;

gynecologic cancers/tumors/carcinomas: e.g., tumors/carcinomas/cancers of the ovary, fallopian tube, peritoneum, cervix, vulva, vagina, uterine body (including endometrium, fundus);

cancers/tumors/carcinomas of the breast: e.g., mammary carcinoma (infiltrating ductal, colloid, lobular invasive, tubular, adenocystic, papillary, medullary, mucinous), hormone receptor positive breast cancer (estrogen receptor positive breast cancer, progesterone receptor positive breast cancer), HER2 positive breast cancer, triple negative breast cancer, Paget's disease of the breast;

cancers/tumors/carcinomas of the endocrine system: e.g., tumors/carcinomas/cancers of the endocrine glands, thyroid gland (thyroid carcinomas/tumors; papillary, follicular, anaplastic, medullary), parathyroid gland (parathyroid carcinoma/tumor), adrenal cortex (adrenal cortical carcinoma/tumors), pituitary gland (including prolactinoma, craniopharyngioma), thymus, adrenal glands, pineal gland, carotid body, islet cell tumors, paraganglion, pancreatic endocrine tumors (PET; non-functional PET, PPoma, gastrinoma, insulinoma, VIPoma, glucagonoma, somatostatinoma, GRFoma, ACTHoma), carcinoid tumors;

sarcomas of the soft tissues: e.g., fibrosarcoma, fibrous histiocytoma, liposarcoma, leiomyosarcoma, rhabdomyosarcoma, angiosarcoma, lymphangiosarcoma, Kaposi's sarcoma, glomus tumor, hemangiopericytoma, synovial sarcoma, giant cell tumor of tendon sheath, solitary fibrous tumor of pleura and peritoneum, diffuse mesothelioma, malignant peripheral nerve sheath tumor (MPNST), granular cell tumor, clear cell sarcoma, melanocytic schwannoma, plexosarcoma, neuroblastoma, ganglioneuroblastoma, neuroepithelioma, extraskeletal Ewing's sarcoma, paraganglioma, extraskeletal chondrosarcoma, extraskeletal osteosarcoma, mesenchymoma, alveolar soft part sarcoma, epithelioid sarcoma, extrarenal rhabdoid tumor, desmoplastic small cell tumor;

sarcomas of the bone: e.g., myeloma, reticulum cell sarcoma, chondrosarcoma (including central, peripheral, clear cell, mesenchymal chondrosarcoma), osteosarcoma (including parosteal, periosteal, high-grade surface, small cell, radiation-induced osteosarcoma, Paget's sarcoma), Ewing's tumor, malignant giant cell tumor, adamantinoma, (fibrous) histiocytoma, fibrosarcoma, chordoma, small round cell sarcoma, hemangioendothelioma, hemangiopericytoma, osteochondroma, osteoid osteoma, osteoblastoma, eosinophilic granuloma, chondroblastoma;

mesothelioma: e.g., pleural mesothelioma, peritoneal mesothelioma;

cancers of the skin: e.g., basal cell carcinoma, squamous cell carcinoma, Merkel's cell carcinoma, melanoma (including cutaneous, superficial spreading, lentigo maligna, acral lentiginous, nodular, intraocular melanoma), actinic keratosis, eyelid cancer;

neoplasms of the peripheral and central nervous system and brain: e.g., astrocytoma (cerebral, cerebellar, diffuse, fibrillary, anaplastic, pilocytic, protoplasmic, gemistocytary), glioblastoma, gliomas, oligodendrogliomas, oligoastrocytomas, ependymomas, ependymoblastomas, choroid plexus tumors, medulloblastomas, meningiomas, schwannomas, hemangioblastomas, hemangiomas, hemangiopericytomas, neuromas, ganglioneuromas, neuroblastomas, retinoblastomas, neurinomas (e.g., acoustic), spinal axis tumors, neurogenic tumors (including neurofibroma, neurilemoma, malignant schwannoma, neurosarcoma, ganglioneuroblastoma, ganglioneuroma, neuroblastoma, pheochromocytoma, paraganglioma), germ cell tumors (including seminoma, teratoma, non-seminoma), thymic tumors (including thymoma, thymolipoma, thymic carcinoma, thymic carcinoid), mesenchymal tumors (including fibroma, fibrosarcoma, lipoma, liposarcoma, myxoma, mesothelioma, leiomyoma, leiomyosarcoma, rhabdomyosarcoma, xanthogranuloma, mesenchymoma, hemangioma, hemangioendothelioma, hemangiopericytoma, lymphangioma, lymphangiopericytoma, lymphangiomyoma);

lymphomas and leukemias: e.g., B-cell non-Hodgkin lymphomas (NHL) (including small lymphocytic lymphoma (SLL), lymphoplasmacytoid lymphoma (LPL), mantle cell lymphoma (MCL), follicular lymphoma (FL), diffuse large cell lymphoma (DLCL), Burkitts lymphoma (BL)), Burkitt leukemia, T-cell non-Hodgkin lymphomas (including anaplastic large cell lymphoma (ALCL), adult T-cell leukemia/lymphoma (ATLL), cutaneous T-cell lymphoma (CTCL), peripheral T-cell lymphoma (PTCL)), lymphoblastic T-cell lymphoma (T-LBL), adult T-cell lymphoma, lymphoblastic B-cell lymphoma (B-LBL), immunocytoma, chronic B-cell lymphocytic leukemia (B-CLL), chronic T-cell lymphocytic leukemia (T-CLL) B-cell small lymphocytic lymphoma (B-SLL), cutaneous T-cell lymphoma (CTLC), primary central nervous system lymphoma (PCNSL), immunoblastoma, Hodgkin's disease (HD) (including nodular lymphocyte predominance HD (NLPHD), nodular sclerosis HD (NSHD), mixed-cellularity HD (MCHD), lymphocyte-rich classic HD, lymphocyte-depleted HD (LDHD)), large granular lymphocyte leukemia (LGL), chronic myelogenous leukemia (CML), acute myelogenous/myeloid leukemia (AML), acute lymphatic/lymphoblastic leukemia (ALL), acute promyelocytic leukemia (APL), chronic lymphocytic/lymphatic leukemia (CLL), prolymphocytic leukemia (PLL), hairy cell leukemia, chronic myelogenous/myeloid leukemia (CML), myeloma, plasmacytoma, multiple myeloma (MM), plasmacytoma, myelodysplastic syndromes (MDS), chronic myelomonocytic leukemia (CMML), JMML (juvenile my elomonocytic leukemia), acute leukemia of ambiguous lineage, myeloproliferative neoplasms, blastic plasmacytoid dendritic cell neoplasm, early T-cell precursor leukemia, natural killer cell leukemia/lymphoma, myeloid/lymphoid neoplasms with eosinophilia, myeloid sarcoma, transient abnormal myelopoiesis; and

cancers of unknown primary site (CUP).

All cancers/tumors/carcinomas mentioned above which are characterized by their specific location/origin in the body are meant to include both the primary tumors and the metastatic tumors derived therefrom.

All cancers/tumors/carcinomas mentioned above may be further differentiated by their histopathological classification:

epithelial cancers, e.g., squamous cell carcinoma (SCC) (carcinoma in situ, superficially invasive, verrucous carcinoma, pseudosarcoma, anaplastic, transitional cell, lymphoepithelial), adenocarcinoma (AC) (well-differentiated, mucinous, papillary, pleomorphic giant cell, ductal, small cell, signet-ring cell, spindle cell, clear cell, oat cell, colloid, adenosquamous, mucoepidermoid, adenoid cystic), mucinous cystadenocarcinoma, acinar cell carcinoma, large cell carcinoma, small cell carcinoma, neuroendocrine tumors (small cell carcinoma, paraganglioma, carcinoid); oncocytic carcinoma; and

nonepithilial and mesenchymal cancers, e.g., sarcomas (fibrosarcoma, chondrosarcoma, rhabdomyosarcoma, leiomyosarcoma, hemangiosarcoma, giant cell sarcoma, lymphosarcoma, fibrous histiocytoma, liposarcoma, angiosarcoma, lymphangiosarcoma, neurofibrosarcoma), lymphoma, melanoma, germ cell tumors, hematological neoplasms, mixed and undifferentiated carcinomas.

The compounds of the present disclosure may be used in therapeutic regimens in the context of first line, second line, or any further line treatments.

The compounds of the invention may be used for the prevention, short-term or long-term treatment of the above-mentioned diseases, optionally also in combination with radiotherapy and/or surgery and/or other compounds.

Of course, the above also includes the use of the compounds of the present disclosure in various methods of treating the above diseases by administering a therapeutically effective dose to a patient in need thereof, as well as the use of these compounds for the manufacture of medicaments for the treatment of such diseases, as well as pharmaceutical compositions including such compounds of the invention, as well as the preparation and/or manufacture of medicaments including such compounds of the invention, and the like.

### Additional Methods of Using the Disclosed Compounds

One aspect of the present disclosure relates to a method of inhibiting SOS1 in a subject in need thereof, comprising administering to the subject a SOS1 inhibitor of the present invention, or a pharmaceutically acceptable salt, solvate, hydrate, tautomer, or isomer thereof.

Another aspect of the present disclosure relates to a method of treating or preventing a disease that is effected or characterized by modification of the interaction of SOS1 and a RAS-family protein and/or RAC1 in a subject in need thereof. The method involves administering to a patient in need of treatment for diseases or disorders associated with SOS1 modulation an effective amount of a compound of any formula disclosed herein, or a pharmaceutically acceptable salt, solvate, isomer, prodrug, or tautomer thereof.

In certain embodiments, a method is provided of inhibiting the interaction of SOS1 and a RAS-family protein in a cell or inhibiting the interaction of SOS1 and RAC1 in a cell, comprising administering to the cell a compound of any formula disclosed herein, or a pharmaceutically acceptable salt, solvate, hydrate, tautomer, or isomer thereof, and a pharmaceutically acceptable carrier.

In certain embodiments, a method is provided of treating or preventing cancer in a subject in need thereof, comprising administering to the subject an effective amount of a compound of any formula disclosed herein, or a pharmaceutically acceptable salt, solvate, hydrate, tautomer, or isomer thereof.

In certain embodiments, the disease can be, but is not limited to, cancer. In certain embodiments, the disease or cancer is selected from the group consisting of pancreatic cancer, lung cancer, colorectal cancer, cholangiocarcinoma, multiple myeloma, melanoma, uterine cancer, endometrial cancer, thyroid cancer, acute myeloid leukemia, JMML (juvenile mvelornonocytic leukermia), acute lymphoblastic leukemia/lymphoma, lymphomas, tumors of the central and peripheral nervous system, epithelial and nonepithelial tumors and mesenchymal tumor, bladder cancer, urothelial cancer, gastric cancer, cervical cancer, head and neck squamous cell carcinoma, diffuse large B cell lymphoma, esophageal cancer, chronic lymphocytic leukemia, hepatocellular cancer, breast cancer, ovarian cancer, prostate cancer, glioblastoma, renal cancer and sarcomas.

In certain embodiments, the disease can be, but is not limited to, cancer. In certain embodiments, the disease or cancer is selected from the group consisting of pancreatic cancer, lung cancer, colorectal cancer, cholangiocarcinoma, multiple myeloma, melanoma, uterine cancer, endometrial cancer, thyroid cancer, acute myeloid leukemia, ladder cancer, urothelial cancer, gastric cancer, cervical cancer, head and neck squamous cell carcinoma, diffuse large B cell lymphoma, esophageal cancer, chronic lymphocytic leukemia, hepatocellular cancer, breast cancer, ovarian cancer, prostate cancer, glioblastoma, renal cancer and sarcomas.

In certain embodiments, the disease can be, but is not limited to, a RASopathy. In certain embodiments, the RASopathy is selected from the group consisting of Neurofibromatosis type 1 (NF1), Noonan Syndrome (NS), Noonan Syndrome with Multiple Lentigines (NSML), Capillary Malformation-Arteriovenous Malformation Syndrome (CM-AVM), Costello Syndrome (CS), Cardio-Facio-Cutaneous Syndrome (CFC), Legius Syndrome, and Hereditary gingival fibromatosis.

Another aspect of the present disclosure is directed to a method of inhibiting SOS1. The method involves administering to a patient in need thereof an effective amount of a compound of any formula disclosed herein, or a pharmaceutically acceptable salt, solvate, isomer, prodrug, or tautomer thereof

The present disclosure relates to compositions capable of modulating the activity of (e.g., inhibiting) SOS1. The present disclosure also relates to the therapeutic use of such compounds.

The disclosed compound can be administered in effective amounts to treat or prevent a disorder and/or prevent the development thereof in subjects.

Another aspect of the present disclosure relates to a compound of any formula disclosed herein, or a pharmaceutically acceptable salt, hydrate, solvate, prodrug, stereoisomer, or tautomer thereof, for use in treating or preventing a disease that is affected by modification of the interaction of SOS1 and a RAS-family protein and/or RAC1. Another aspect of the present disclosure relates to a compound of any formula disclosed herein, or a pharmaceutically acceptable salt, hydrate, solvate, prodrug, stereoisomer, or tautomer thereof, for use in treating or preventing a disease that is characterized by inhibition of the interaction of SOS1 with a RAS-family protein or the interaction of SOS1 with RAC1.

Another aspect of the present disclosure relates to a compound of any formula disclosed herein, or a pharmaceutically acceptable salt, hydrate, solvate, prodrug, stereoisomer, or tautomer thereof, for use in treating or preventing a disease, wherein the treating or preventing is effected or characterized by inhibition of the interaction of SOS1 and a RAS-family protein or by inhibition of the interaction of SOS1 and RA.

Another aspect of the present disclosure relates to a compound of any formula disclosed herein, or a pharmaceutically acceptable salt, hydrate, solvate, prodrug, stereoisomer, or tautomer thereof, for use inhibiting the binding of hSOS1 to H- or N- or K-RAS including their clinically known mutations and which inhibits the nucleotide exchange reaction catalyzed by hSOS1 in the presence of a concentration of 20 μM or lower, but which are substantially inactive against EGFR-kinase at concentrations of 20 μM or lower for the preparation of a medicament for the treatment or prophylaxis of a hyperproliferative disorder.

Another aspect of the present disclosure relates to a compound of any formula disclosed herein, or a pharmaceutically acceptable salt, hydrate, solvate, prodrug, stereoisomer, or tautomer thereof, for the manufacture of a medicament for use inhibiting the binding of hSOS1 specifically to K-RAS G12C protein or another Ras mutant, as described herein, and which inhibits the nucleotide exchange reaction catalyzed by hSOS1 in the presence of a concentration of 20 μM or lower, but which are substantially inactive against EGFR-kinase at concentrations of 20 μM or lower for the preparation of a medicament for the treatment or prophylaxis of a hyperproliferative disorder.

In another aspect, the present disclosure relates to the use of a compound of any formula disclosed herein, or a pharmaceutically acceptable salt, hydrate, solvate, prodrug, stereoisomer, or tautomer thereof, in the manufacture of a medicament for treating or preventing a disease.

Administration of the disclosed compounds can be accomplished via any mode of administration for therapeutic agents. These modes include systemic or local administration such as oral, nasal, parenteral, intravenous, transdermal, subcutaneous, vaginal, buccal, rectal or topical administration modes. Likewise, they can also be administered in intravenous (both bolus and infusion), intraperitoneal, subcutaneous or intramuscular form, and all using forms well known to those skilled in the pharmaceutical arts

Depending on the intended mode of administration, the disclosed compounds or pharmaceutical compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices.

Illustrative pharmaceutical compositions are tablets and gelatin capsules comprising a compound of the disclosure and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, alginic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, the disclosed compound is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

The disclosed compounds can be also formulated as a suppository that can be prepared from fatty emulsions or suspensions; using polyalkylene glycols such as propylene glycol, as the carrier.

The disclosed compounds can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described for instance in U.S. Pat. No. 5,262,564, the contents of which are hereby incorporated by reference.

Disclosed compounds can also be delivered by the use of monoclonal antibodies as individual carriers to which the disclosed compounds are coupled. The disclosed compounds can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the disclosed compounds can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels. In one embodiment, disclosed compounds are not covalently bound to a polymer, e.g., a polycarboxylic acid polymer, or a polyacrylate.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

Another aspect of the present disclosure relates to a pharmaceutical composition comprising a compound of the present disclosure and a pharmaceutically acceptable carrier. The pharmaceutically acceptable carrier can further include an excipient, diluent, or surfactant.

Compositions can be prepared according to conventional mixing, granulating or coating methods, respectively, and the present pharmaceutical compositions can contain from about 0.1% to about 99%, from about 5% to about 90%, or from about 1% to about 20% of the disclosed compound by weight or volume.

The dosage regimen utilizing the disclosed compound is selected in accordance with a variety of factors including type, species, age, weight, sex, and medical condition of the patient; the severity of the condition to be treated; the route of administration; the renal or hepatic function of the patient; and the particular disclosed compound employed. A physician or veterinarian of ordinary skill in the art can readily determine and prescribe the effective amount of the drug required to prevent, counter or arrest the progress of the condition.

Effective dosage amounts of the disclosed compounds, when used for the indicated effects, range from about 0.5 mg to about 5000 mg of the disclosed compound as needed to treat the condition. Compositions for in vivo or in vitro use can contain about 0.5, 5, 20, 50, 75, 100, 150, 250, 500, 750, 1000, 1250, 2500, 3500, or 5000 mg of the disclosed compound, or, in a range of from one amount to another amount in the list of doses. In one embodiment, the compositions are in the form of a tablet that can be scored.

### Combination Therapy

The methods of the invention may include a compound of the invention used alone or in combination with one or more additional therapies (e.g., non-drug treatments or therapeutic agents). Combination therapy may, for example, combine two therapies or may combine three therapies (e.g., a triple therapy of three therapeutic agents), or more. The dosages of one or more of the additional therapies (e.g., non-drug treatments or therapeutic agents) may be reduced from standard dosages when administered alone. For example, doses may be determined empirically from drug combinations and permutations or may be deduced by isobolographic analysis (e.g., Black et al., Neurology 65:S3-S6 (2005)).

A compound of the present invention may be administered before, after, or concurrently with one or more of such additional therapies. When combined, dosages of a compound of the invention and dosages of the one or more additional therapies (e.g., non-drug treatment or therapeutic agent) provide a therapeutic effect (e.g., synergistic or additive therapeutic effect). A compound of the present invention and an additional therapy, such as an anti-cancer agent, may be administered together, such as in a unitary pharmaceutical composition, or separately and, when administered separately, this may occur simultaneously or sequentially. Such sequential administration may be close or remote in time.

In some embodiments, the additional therapy is the administration of side-effect limiting agents (e.g., agents intended to lessen the occurrence or severity of side effects of treatment. For example, in some embodiments, the compounds of the present invention can also be used in combination with a therapeutic agent that treats nausea. Examples of agents that can be used to treat nausea include: dronabinol, granisetron, metoclopramide, ondansetron, and prochlorperazine, or pharmaceutically acceptable salts thereof.

In some embodiments, the one or more additional therapies includes a non-drug treatment (e.g., surgery or radiation therapy). In some embodiments, the one or more additional therapies includes a therapeutic agent (e.g., a compound or biologic that is an anti-angiogenic agent, signal transduction inhibitor, antiproliferative agent, glycolysis inhibitor, or autophagy inhibitor). In some embodiments, the one or more additional therapies includes a non-drug treatment (e.g., surgery or radiation therapy) and a therapeutic agent (e.g., a compound or biologic that is an anti-angiogenic agent, signal transduction inhibitor, antiproliferative agent, glycolysis inhibitor, or autophagy inhibitor). In other embodiments, the one or more additional therapies includes two therapeutic agents. In still other embodiments, the one or more additional therapies includes three therapeutic agents. In some embodiments, the one or more additional therapies includes four or more therapeutic agents.

### Non-Drug Therapies

Examples of non-drug treatments include, but are not limited to, radiation therapy, cryotherapy, hyperthermia, surgery (e.g., surgical excision of tumor tissue), and T cell adoptive transfer (ACT) therapy.

In some embodiments, the compounds of the invention may be used as an adjuvant therapy after surgery. In some embodiments, the compounds of the invention may be used as a neo-adjuvant therapy prior to surgery.

Radiation therapy may be used for inhibiting abnormal cell growth or treating a hyperproliferative disorder, such as cancer, in a subject (e.g., mammal (e.g., human)). Techniques for administering radiation therapy are known in the art. Radiation therapy can be administered through one of several methods, or a combination of methods, including, without limitation, external-beam therapy, internal radiation therapy, implant radiation, stereotactic radiosurgery, systemic radiation therapy, radiotherapy and permanent or temporary interstitial brachy therapy. The term “brachy therapy,” as used herein, refers to radiation therapy delivered by a spatially confined radioactive material inserted into the body at or near a tumor or other proliferative tissue disease site. The term is intended, without limitation, to include exposure to radioactive isotopes (e.g., At-211, 1-131, 1-125, Y-90, Re-186, Re-188, Sm-153, Bi-212, P-32, and radioactive isotopes of Lu). Suitable radiation sources for use as a cell conditioner of the present invention include both solids and liquids. By way of non-limiting example, the radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, or Y-90. Moreover, the radionuclide(s) can be embodied in a gel or radioactive micro spheres.

In some embodiments, the compounds of the present invention can render abnormal cells more sensitive to treatment with radiation for purposes of killing or inhibiting the growth of such cells. Accordingly, this invention further relates to a method for sensitizing abnormal cells in a mammal to treatment with radiation which comprises administering to the mammal an amount of a compound of the present invention, which amount is effective to sensitize abnormal cells to treatment with radiation. The amount of the compound in this method can be determined according to the means for ascertaining effective amounts of such compounds described herein. In some embodiments, the compounds of the present invention may be used as an adjuvant therapy after radiation therapy or as a neo-adjuvant therapy prior to radiation therapy.

In some embodiments, the non-drug treatment is a T cell adoptive transfer (ACT) therapy. In some embodiments, the T cell is an activated T cell. The T cell may be modified to express a chimeric antigen receptor (CAR). CAR modified T (CAR-T) cells can be generated by any method known in the art. For example, the CAR-T cells can be generated by introducing a suitable expression vector encoding the CAR to a T cell. Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art may be used. In some embodiments, the T cell is an autologous T cell. Whether prior to or after genetic modification of the T cells to express a desirable protein (e.g., a CAR), the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 7,572,631; 5,883,223; 6,905,874; 6,797,514; and 6,867,041.

### Therapeutic Agents

A therapeutic agent may be a compound used in the treatment of cancer or symptoms associated therewith.

For example, a therapeutic agent may be a steroid. Accordingly, in some embodiments, the one or more additional therapies includes a steroid. Suitable steroids may include, but are not limited to, 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, fiucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts or derivatives thereof.

Further examples of therapeutic agents that may be used in combination therapy with a compound of the present invention include compounds described in the following patents: U.S. Pat. Nos. 6,258,812, 6,630,500, 6,515,004, 6,713,485, 5,521,184, 5,770,599, 5,747,498, 5,990,141, 6,235,764, and 8,623,885, and International Patent Applications WO01/37820, WO01/32651, WO02/68406, WO02/66470, WO02/55501, WO04/05279, WO04/07481, WO04/07458, WO04/09784, WO02/59110, WO99/45009, WO00/59509, WO99/61422, WO00/12089, and WO00/02871.

A therapeutic agent may be a biologic (e.g., cytokine (e.g., interferon or an interleukin such as IL-2)) used in treatment of cancer or symptoms associated therewith. In some embodiments, the biologic is an immunoglobulin-based biologic, e.g., a monoclonal antibody (e.g., a humanized antibody, a fully human antibody, an Fc fusion protein, or a functional fragment thereof) that agonizes a target to stimulate an anti-cancer response or antagonizes an antigen important for cancer. Also included are antibody-drug conjugates.

A therapeutic agent may be a checkpoint inhibitor. In one embodiment, the checkpoint inhibitor is an inhibitory antibody (e.g., a monospecific antibody such as a monoclonal antibody). The antibody may be, e.g., humanized or fully human. In some embodiments, the checkpoint inhibitor is a fusion protein, e.g., an Fc-receptor fusion protein. In some embodiments, the checkpoint inhibitor is an agent, such as an antibody, that interacts with a checkpoint protein. In some embodiments, the checkpoint inhibitor is an agent, such as an antibody, that interacts with the ligand of a checkpoint protein. In some embodiments, the checkpoint inhibitor is an inhibitor (e.g., an inhibitory antibody or small molecule inhibitor) of CTLA-4 (e.g., an anti-CTLA-4 antibody or fusion a protein). In some embodiments, the checkpoint inhibitor is an inhibitor or antagonist (e.g., an inhibitory antibody or small molecule inhibitor) of PD-1. In some embodiments, the checkpoint inhibitor is an inhibitor or antagonist (e.g., an inhibitory antibody or small molecule inhibitor) of PDL-1. In some embodiments, the checkpoint inhibitor is an inhibitor or antagonist (e.g., an inhibitory antibody or Fc fusion or small molecule inhibitor) of PDL-2 (e.g., a PDL-2/Ig fusion protein). In some embodiments, the checkpoint inhibitor is an inhibitor or antagonist (e.g., an inhibitory antibody or small molecule inhibitor) of B7-H3, B7-H4, BTLA, HVEM, TIM3, GAL9, LAG3, VISTA, KIR, 2B4, CD160, CGEN-15049, CHK 1, CHK2, A2aR, B-7 family ligands, or a combination thereof. In some embodiments, the checkpoint inhibitor is pembrolizumab, nivolumab, PDR001 (NVS), REGN2810 (Sanofi/Regeneron), a PD-L1 antibody such as, e.g., avelumab, durvalumab, atezolizumab, pidilizumab, JNJ-63723283 (JNJ), BGB-A317 (BeiGene & Celgene) or a checkpoint inhibitor disclosed in Preusser, M. et al. (2015) Nat. Rev. Neurol., including, without limitation, ipilimumab, tremelimumab, nivolumab, pembrolizumab, AMP224, AMP514/MEDI0680, BMS936559, MED14736, MPDL3280A, MSB0010718C, BMS986016, IMP321, lirilumab, IPH2101, 1-7F9, and KW-6002.

A therapeutic agent may be an agent that treats cancer or symptoms associated therewith (e.g., a cytotoxic agent, non-peptide small molecules, or other compound useful in the treatment of cancer or symptoms associated therewith, collectively, an “anti-cancer agent”). Anti-cancer agents can be, e.g., chemotherapeutics or targeted therapy agents.

Anti-cancer agents include mitotic inhibitors, intercalating antibiotics, growth factor inhibitors, cell cycle inhibitors, enzymes, topoisomerase inhibitors, biological response modifiers, alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Further anti-cancer agents include leucovorin (LV), irenotecan, oxaliplatin, capecitabine, paclitaxel, and doxetaxel. In some embodiments, the one or more additional therapies includes two or more anti-cancer agents. The two or more anti-cancer agents can be used in a cocktail to be administered in combination or administered separately. Suitable dosing regimens of combination anti-cancer agents are known in the art and described in, for example, Saltz et al., Proc. Am. Soc. Clin. Oncol. 18:233a (1999), and Douillard et al., Lancet 355(9209):1041-1047 (2000).

Other non-limiting examples of anti-cancer agents include Gleevec® (Imatinib Mesylate); Kyprolis® (carfilzomib); Velcade® (bortezomib); Casodex (bicalutamide); Iressa® (gefitinib); alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; sarcodictyin A; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, such as calicheamicin gammall and calicheamicin omegall (see, e.g., Agnew, Chem. Intl. Ed Engl. 33:183-186 (1994)); dynemicin such as dynemicin A; bisphosphonates such as clodronate; an esperamicin; neocarzinostatin chromophore and related chromoprotein enediyne antibiotic chromophores, aclacinomysins, actinomycin, authramycin, azaserine, bleomycins, cactinomycin, calicheamicin, carabicin, caminomycin, carminomycin, carzinophilin, chromomycins, dactinomycin, daunorubicin, detorubicin, 6-diazo-5-oxo-L-norleucine, adriamycin (doxorubicin), morpholino-doxorubicin, cyanomorpholino-doxorubicin, 2-pyrrolino-doxorubicin, deoxydoxorubicin, epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenishers such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone such as epothilone B; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes such as T-2 toxin, verracurin A, roridin A and anguidine; urethane; vindesine; dacarbazine; mannomustine; mitobronitol; mitolactol; pipobroman; gacytosine; arabinoside (“Ara-C”); cyclophosphamide; thiotepa; taxoids, e.g., Taxol® (paclitaxel), Abraxane® (cremophor-free, albumin-engineered nanoparticle formulation of paclitaxel), and Taxotere® (doxetaxel); chloranbucil; tamoxifen (Nolvadex™); raloxifene; aromatase inhibiting 4(5)-imidazoles; 4-hydroxytamoxifen; trioxifene; keoxifene; LY 117018; onapristone; toremifene (Fareston®); flutamide, nilutamide, bicalutamide, leuprolide, goserelin; chlorambucil; Gemzar® gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; Navelbine® (vinorelbine); novantrone; teniposide; edatrexate; daunomycin; aminopterin; ibandronate; irinotecan (e.g., CPT-11); topoisomerase inhibitor RFS 2000; difluoromethylomithine (DMFO); retinoids such as retinoic acid; esperamicins; capecitabine (e.g., Xeloda®); and pharmaceutically acceptable salts of any of the above.

Additional non-limiting examples of anti-cancer agents include trastuzumab (Herceptin®), bevacizumab (Avastin®), cetuximab (Erbitux®), rituximab (Rituxan®), Taxol®, Arimidex®, ABVD, avicine, abagovomab, acridine carboxamide, adecatumumab, 17-N-allylamino-17-demethoxygeldanamycin, alpharadin, alvocidib, 3-aminopyridine-2-carboxaldehyde thiosemicarbazone, amonafide, anthracenedione, anti-CD22 immunotoxins, antineoplastics (e.g., cell-cycle nonspecific antineoplastic agents, and other antineoplastics described herein), antitumorigenic herbs, apaziquone, atiprimod, azathioprine, belotecan, bendamustine, BIBW 2992, biricodar, brostallicin, bryostatin, buthionine sulfoximine, CBV (chemotherapy), calyculin, dichloroacetic acid, discodermolide, elsamitrucin, enocitabine, eribulin, exatecan, exisulind, ferruginol, forodesine, fosfestrol, ICE chemotherapy regimen, IT-101, imexon, imiquimod, indolocarbazole, irofulven, laniquidar, larotaxel, lenalidomide, lucanthone, lurtotecan, mafosfamide, mitozolomide, nafoxidine, nedaplatin, olaparib, ortataxel, PAC-1, pawpaw, pixantrone, proteasome inhibitors, rebeccamycin, resiquimod, rubitecan, SN-38, salinosporamide A, sapacitabine, Stanford V, swainsonine, talaporfin, tariquidar, tegafur-uracil, temodar, tesetaxel, triplatin tetranitrate, tris(2-chloroethyl)amine, troxacitabine, uramustine, vadimezan, vinflunine, ZD6126, and zosuquidar.

Further non-limiting examples of anti-cancer agents include natural products such as vinca alkaloids (e.g., vinblastine, vincristine, and vinorelbine), epidipodophyllotoxins (e.g., etoposide and teniposide), antibiotics (e.g., dactinomycin (actinomycin D), daunorubicin, and idarubicin), anthracyclines, mitoxantrone, bleomycins, plicamycin (mithramycin), mitomycin, enzymes (e.g., L-asparaginase which systemically metabolizes L-asparagine and deprives cells which do not have the capacity to synthesize their own asparagine), antiplatelet agents, antiproliferative/antimitotic alkylating agents such as nitrogen mustards (e.g., mechlorethamine, cyclophosphamide and analogs, melphalan, and chlorambucil), ethylenimines and methylmelamines (e.g., hexaamethylmelaamine and thiotepa), CDK inhibitors (e.g., a CDK 4/6 inhibitor such as ribociclib, abemaciclib, or palbociclib), seliciclib, UCN-01, P1446A-05, PD-0332991, dinaciclib, P27-00, AT-7519, RGB286638, and SCH727965), alkyl sulfonates (e.g., busulfan), nitrosoureas (e.g., carmustine (BCNU) and analogs, and streptozocin), trazenes-dacarbazinine (DTIC), antiproliferative/antimitotic antimetabolites such as folic acid analogs, pyrimidine analogs (e.g., fluorouracil, floxuridine, and cytarabine), purine analogs and related inhibitors (e.g., mercaptopurine, thioguanine, pentostatin, and 2-chlorodeoxyadenosine), aromatase inhibitors (e.g., anastrozole, exemestane, and letrozole), and platinum coordination complexes (e.g., cisplatin and carboplatin), procarbazine, hydroxyurea, mitotane, aminoglutethimide, histone deacetylase (HDAC) inhibitors (e.g., trichostatin, sodium butyrate, apicidan, suberoyl anilide hydroamic acid, vorinostat, LBH 589, romidepsin, ACY-1215, and panobinostat), mTOR inhibitors (e.g., vistusertib, temsirolimus, everolimus, ridaforolimus, and sirolimus), KSP(Eg5) inhibitors (e.g., Array 520), DNA binding agents (e.g., Zalypsis®), PI3K inhibitors such as PI3K delta inhibitor (e.g., GS-1101 and TGR-1202), PI3K delta and gamma inhibitor (e.g., CAL-130), copanlisib, alpelisib and idelalisib; multi-kinase inhibitor (e.g., TG02 and sorafenib), hormones (e.g., estrogen) and hormone agonists such as leutinizing hormone releasing hormone (LHRH) agonists (e.g., goserelin, leuprolide and triptorelin), BAFF-neutralizing antibody (e.g., LY2127399), IKK inhibitors, p38MAPK inhibitors, anti-IL-6 (e.g., CNT0328), telomerase inhibitors (e.g., GRN 163L), aurora kinase inhibitors (e.g., MLN8237), cell surface monoclonal antibodies (e.g., anti-CD38 (HUMAX-CD38), anti-CSl (e.g., elotuzumab), HSP90 inhibitors (e.g., 17 AAG and KOS 953), PI3K/Akt inhibitors (e.g., perifosine), Akt inhibitors (e.g., GSK-2141795), PKC inhibitors (e.g., enzastaurin), FTIs (e.g., Zamestra™), anti-CD138 (e.g., BT062), Torcl/2 specific kinase inhibitors (e.g., INK128), ER/UPR targeting agents (e.g., MKC-3946), cFMS inhibitors (e.g., ARRY-382), JAK1/2 inhibitors (e.g., CYT387), PARP inhibitors (e.g., olaparib and veliparib (ABT-888)), and BCL-2 antagonists.

In some embodiments, an anti-cancer agent is selected from mechlorethamine, camptothecin, ifosfamide, tamoxifen, raloxifene, gemcitabine, Navelbine®, sorafenib, or any analog or derivative variant of the foregoing.

In some embodiments, an anti-cancer agent is an ALK inhibitor. Non-limiting examples of ALK inhibitors include ceritinib, TAE-684 (NVP-TAE694), PF02341066 (crizotinib or 1066), alectinib; brigatinib; entrectinib; ensartinib (X-396); lorlatinib; ASP3026; CEP-37440; 4SC-203; TL-398; PLB1003; TSR-011; CT-707; TPX-0005, and AP26113. Additional examples of ALK kinase inhibitors are described in examples 3-39 of WO05016894.

In some embodiments, an anti-cancer agent is an inhibitor of a member downstream of a Receptor Tyrosine Kinase (RTK)/Growth Factor Receptor (e.g., a SHP2 inhibitor (e.g., SHP099, TNO155, RMC-4550, RMC-4630, JAB-3068), another SOS1 inhibitor (e.g., BI-1701963), a Raf inhibitor, a MEK inhibitor, an ERK inhibitor, a PT3K inhibitor, a PTEN inhibitor, an AKT inhibitor, or an mTOR inhibitor (e.g., mTORCl inhibitor or mTORC2 inhibitor). In some embodiments, the anti-cancer agent is JAB-3312. In some embodiments, an anti-cancer agent is a Ras inhibitor (e.g., AMG 510, MRTX1257, LY349946, MRTX849, ARS-3248 (JNJ-74699157), or ARS-1620), or a Ras vaccine, or another therapeutic modality designed to directly or indirectly decrease the oncogenic activity of Ras.

In some embodiments, the Ras protein is wild-type. In some embodiments, the cancer comprises a Ras mutation. In some embodiments, a mutation is selected from:
(a) the following K-Ras mutants: G12D, G12V, G12C, G13D, G12R, G12A, Q61H, G12S, A146T, G13C, Q61L, Q61R, K117N, A146V, G12F, Q61K, L19F, Q22K, V14I, A59T, A146P, G13R, G12L, or G13V, and combinations thereof;
(b) the following H-Ras mutants: Q61R, G13R, Q61K, G12S, Q61L, G12D, G13V, G13D, G12C, K117N, A59T, G12V, G13C, Q61H, G13S, A18V, D119N, G13N, A146T, A66T, G12A, A146V, G12N, or G12R, and combinations thereof, and
(c) the following N-Ras mutants: Q61R, Q61K, G12D, Q61L, Q61H, G13R, G13D, G12S, G12C, G12V, G12A, G13V, G12R, P185S, G13C, A146T, G60E, Q61P, A59D, E132K, E49K, T50I, A146V, or A59T, and combinations thereof;
   or a combination of any of the foregoing (e.g., both K-Ras G12C and K-Ras G13C). In some embodiments, the cancer comprises a Ras mutation selected from the group consisting of G12C, G13C, G12A, G12D, G13D, G12S, G13S, G12V and G13V.

In some embodiments, a therapeutic agent that may be combined with a compound of the present invention is an inhibitor of the MAP kinase (MAPK) pathway (or “MAPK inhibitor”). MAPK inhibitors include, but are not limited to, one or more MAPK inhibitor described in Cancers (Basel) 2015 September; 7(3): 1758-1784. For example, the MAPK inhibitor may be selected from one or more of trametinib, binimetinib, selumetinib, cobimetinib, LErafAON (NeoPharm), ISIS 5132; vemurafenib, pimasertib, TAK733, R04987655 (CH4987655); CI-1040; PD-0325901; CH5126766; MAP855; AZD6244; refametinib (RDEA 119/BAY 86-9766); GDC-0973/XL581; AZD8330 (ARRY-424704/ARRY-704); R05126766 (Roche, described in PLoS One. 2014 Nov. 25; 9(11)); and GSK1120212 (or JTP-74057, described in Clin Cancer Res. 2011 Mar. 1; 17(5):989-1000).

In some embodiments, an anti-cancer agent is a disrupter or inhibitor of the RAS-RAF-ERK or PI3K-AKT-TOR or PI3K-AKT signaling pathways. The PI3K/AKT inhibitor may include, but is not limited to, one or more PI3K/AKT inhibitor described in Cancers (Basel) 2015 September; 7(3): 1758-1784. For example, the PI3K/AKT inhibitor may be selected from one or more of NVP-BEZ235; BGT226; XL765/SAR245409; SF1126; GDC-0980; PI-103; PF-04691502; PKI-587; GSK2126458.

In some embodiments, an anti-cancer agent is a PD-1 or PD-L1 antagonist.

In some embodiments, additional therapeutic agents include EGFR inhibitors, IGF-1R inhibitors, MEK inhibitors, PI3K inhibitors, AKT inhibitors, TOR inhibitors, MCL-1 inhibitors, BCL-2 inhibitors, SHP2 inhibitors, proteasome inhibitors, and immune therapies.

IGF-1R inhibitors include linsitinib, or a pharmaceutically acceptable salt thereof.

EGFR inhibitors include, but are not limited to, small molecule antagonists, antibody inhibitors, or specific antisense nucleotide or siRNA. Useful antibody inhibitors of EGFR include cetuximab (Erbitux®), panitumumab (Vectibix®), zalutumumab, nimotuzumab, and matuzumab. Further antibody-based EGFR inhibitors include any anti-EGFR antibody or antibody fragment that can partially or completely block EGFR activation by its natural ligand. Non-limiting examples of antibody-based EGFR inhibitors include those described in Modjtahedi et al., Br. J. Cancer 1993, 67:247-253; Teramoto et al., Cancer 1996, 77:639-645; Goldstein et al., Clin. Cancer Res. 1995, 1:1311-1318; Huang et al., 1999, Cancer Res. 15:59(8):1935-40; and Yang et al., Cancer Res. 1999, 59:1236-1243. The EGFR inhibitor can be monoclonal antibody Mab E7.6.3 (Yang, 1999 supra), or Mab C225 (ATCC Accession No. HB-8508), or an antibody or antibody fragment having the binding specificity thereof.

Small molecule antagonists of EGFR include gefitinib (Iressa®), erlotinib (Tarceva®), and lapatinib (TykerB®). See, e.g., Yan et al., Pharmacogenetics and Pharmacogenomics In Oncology Therapeutic Antibody Development, BioTechniques 2005, 39(4):565-8; and Paez et al., EGFR Mutations In Lung Cancer Correlation With Clinical Response To Gefitinib Therapy, Science 2004, 304(5676):1497-500. Further non-limiting examples of small molecule EGFR inhibitors include any of the EGFR inhibitors described in the following patent publications, and all pharmaceutically acceptable salts of such EGFR inhibitors: EP 0520722; EP 0566226; WO96/33980; U.S. Pat. No. 5,747,498; WO96/30347; EP 0787772; WO97/30034; WO97/30044; WO97/38994; WO97/49688; EP 837063; WO98/02434; WO97/38983; WO95/19774; WO95/19970; WO97/13771; WO98/02437; WO98/02438; WO97/32881; DE 19629652; WO98/33798; WO97/32880; WO97/32880; EP 682027; WO97/02266; WO97/27199; WO98/07726; WO97/34895; WO96/31510; WO98/14449; WO98/14450; WO98/14451; WO95/09847; WO97/19065; WO98/17662; U.S. Pat. Nos. 5,789,427; 5,650,415; 5,656,643; WO99/35146; WO99/35132; WO99/07701; and WO92/20642. Additional non-limiting examples of small molecule EGFR inhibitors include any of the EGFR inhibitors described in Traxler et al., Exp. Opin. Ther. Patents 1998, 8(12):1599-1625. In some embodiments, an EGFR inhibitor is osimertinib.

MEK inhibitors include, but are not limited to, pimasertib, selumetinib, cobimetinib (Cotellic®), trametinib (Mekinist®), and binimetinib (Mektovi®). In some embodiments, a MEK inhibitor targets a MEK mutation that is a Class I MEKI mutation selected from D67N; P124L; P124S; and L177V. In some embodiments, the MEK mutation is a Class II MEKI mutation selected from AE51-Q58; AF53-Q58; E203K; L177M; C121S; F53L; K57E; Q56P; and K57N.

PI3K inhibitors include, but are not limited to, wortmannin; 17-hydroxywortmannin analogs described in WO06/044453; 4-[2-(1H-Indazol-4-yl)-6-[[4-(methylsulfonyl)piperazin-1-yl]methyl]thieno[3,2-d]pyrimidin-4-yl]morpholine (also known as pictilisib or GDC-0941 and described in WO09/036082 and WO09/055730); 2-methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in WO06/122806); (S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one (described in WO08/070740); LY294002 (2-(4-morpholinyl)-8-phenyl-4H-1-benzopyran-4-one (available from Axon Medchem); PI 103 hydrochloride (3-[4-(4-morpholinylpyrido-[3′,2′:4,5]furo[3,2-d]pyrimidin-2-yl] phenol hydrochloride (available from Axon Medchem); PIK 75 (2-methyl-5-nitro-2-[(6-bromoimidazo[1,2-a]pyridin-3-yl)methylene]-1-methylhydrazide-benzenesulfonic acid, monohydrochloride) (available from Axon Medchem); PIK 90 (N-(7,8-dimethoxy-2,3-dihydro-imidazo[1,2-c]quinazolin-5-yl)-nicotinamide (available from Axon Medchem); AS-252424 (5-[1-[5-(4-fluoro-2-hydroxy-phenyl)-furan-2-yl]-meth-(Z)-ylidene]-thiazolidine-2,4-dione (available from Axon Medchem); TGX-221 (7-methyl-2-(4-morpholinyl)-9-[1-(phenylamino)ethyl]-4H-pyrido-[1,2-a]pyrimidin-4-one (available from Axon Medchem); XL-765; and XL-147. Other PI3K inhibitors include demethoxyviridin, perifosine, CAL101, PX-866, BEZ235, SF1126, INK1117, IPI-145, BKM120, XL147, XL765, Palomid 529, GSK1059615, ZSTK474, PWT33597, IC87114, TGI 00-115, CAL263, PI-103, GNE-477, CUDC-907, and AEZS-136.

AKT inhibitors include, but are not limited to, Akt-1-1 (inhibits Aktl) (Barnett et al., Biochem. J. 2005, 385(Pt. 2): 399-408); Akt-1-1,2 (inhibits Akl and 2) (Barnett et al., Biochem. J. 2005, 385(Pt. 2): 399-408); API-59CJ-Ome (e.g., Jin et al., Br. J. Cancer 2004, 91:1808-12); 1-H-imidazo[4,5-c]pyridinyl compounds (e.g., WO 05/011700); indole-3-carbinol and derivatives thereof (e.g., U.S. Pat. No. 6,656,963; Sarkar and Li J Nutr. 2004, 134(12 Suppl):34935-34985); perifosine (e.g., interferes with Akt membrane localization; Dasmahapatra et al. Clin. Cancer Res. 2004, 10(15):5242-52); phosphatidylinositol ether lipid analogues (e.g., Gills and Dennis Expert. Opin. Investig. Drugs 2004, 13:787-97); and triciribine (TCN or API-2 or NCI identifier: NSC 154020; Yang et al., Cancer Res. 2004, 64:4394-9).

mTOR inhibitors include, but are not limited to, ATP-competitive mTORC1/mTORC2 inhibitors, e.g., PI-103, PP242, PP30; Torin 1; FKBP12 enhancers; 4H-1-benzopyran-4-one derivatives; and rapamycin (also known as sirolimus) and derivatives thereof, including: temsirolimus (Torisel®); everolimus (Afinitor®; WO94/09010); ridaforolimus (also known as deforolimus or AP23573); rapalogs, e.g., as disclosed in WO98/02441 and WO01/14387, e.g., AP23464 and AP23841; 40-(2-hydroxyethyl)rapamycin; 40-[3-hydroxy(hydroxymethyl)methylpropanoate]-rapamycin (also known as CC1779); 40-epi-(tetrazolyt)-rapamycin (also called ABT578); 32-deoxorapamycin; 16-pentynyloxy-32(S)-dihydrorapanycin; derivatives disclosed in WO05/005434; derivatives disclosed in U.S. Pat. Nos. 5,258,389, 5,118,677, 5,118,678, 5,100,883, 5,151,413, 5,120,842, and 5,256,790, and in WO94/090101, WO92/05179, WO93/111130, WO94/02136, WO94/02485, WO95/14023, WO94/02136, WO95/16691, WO96/41807, WO96/41807, and WO2018204416; and phosphorus-containing rapamycin derivatives (e.g., WO05/016252). In some embodiments, the mTOR inhibitor is a bisteric inhibitor (see, e.g., WO2018204416, WO2019212990 and WO2019212991), such as RMC-5552.

BRAF inhibitors that may be used in combination with compounds of the invention include, for example, vemurafenib, dabrafenib, and encorafenib. A BRAF may comprise a Class 3 BRAF mutation. In some embodiments, the Class 3 BRAF mutation is selected from one or more of the following amino acid substitutions in human BRAF: D287H; P367R; V459L; G466V; G466E; G466A; S467L; G469E; N581S; N5811; D594N; D594G; D594A; D594H; F595L; G596D; G596R and A762E.

MCL-1 inhibitors include, but are not limited to, AMG-176, MIK665, and S63845. The myeloid cell leukemia-1 (MCL-1) protein is one of the key anti-apoptotic members of the B-cell lymphoma-2 (BCL-2) protein family. Over-expression of MCL-1 has been closely related to tumor progression as well as to resistance, not only to traditional chemotherapies but also to targeted therapeutics including BCL-2 inhibitors such as ABT-263.

In some embodiments, the additional therapeutic agent is a SHP2 inhibitor. SHP2 is a non-receptor protein tyrosine phosphatase encoded by the PTPN11 gene that contributes to multiple cellular functions including proliferation, differentiation, cell cycle maintenance and migration. SHP2 has two N-terminal Src homology 2 domains (N-SH2 and C-SH2), a catalytic domain (PTP), and a C-terminal tail. The two SH2 domains control the subcellular localization and functional regulation of SHP2. The molecule exists in an inactive, self-inhibited conformation stabilized by a binding network involving residues from both the N-SH2 and PTP domains. Stimulation by, for example, cytokines or growth factors acting through receptor tyrosine kinases (RTKs) leads to exposure of the catalytic site resulting in enzymatic activation of SHP2.

SHP2 is involved in signaling through the RAS-mitogen-activated protein kinase (MAPK), the JAK-STAT or the phosphoinositol 3-kinase-AKT pathways. Mutations in the PTPN11 gene and subsequently in SHP2 have been identified in several human developmental diseases, such as Noonan Syndrome and Leopard Syndrome, as well as human cancers, such as juvenile myelomonocytic leukemia, neuroblastoma, melanoma, acute myeloid leukemia and cancers of the breast, lung and colon. Some of these mutations destabilize the auto-inhibited conformation of SHP2 and promote autoactivation or enhanced growth factor driven activation of SHP2. SHP2, therefore, represents a highly attractive target for the development of novel therapies for the treatment of various diseases including cancer. A SHP2 inhibitor (e.g., RMC-4550 or SHP099) in combination with a RAS pathway inhibitor (e.g., a MEK inhibitor) have been shown to inhibit the proliferation of multiple cancer cell lines in vitro (e.g., pancreas, lung, ovarian and breast cancer). Thus, combination therapy involving a SHP2 inhibitor with a RAS pathway inhibitor could be a general strategy for preventing tumor resistance in a wide range of malignancies, and may form the basis of a triple combination inhibitor with a SOS1 inhibitor.

Non-limiting examples of such SHP2 inhibitors that are known in the art, include: Chen et al. Mol Pharmacol. 2006, 70, 562; Sarver et al., J. Med. Chem. 2017, 62, 1793; Xie et al., J. Med. Chem. 2017, 60, 113734; and Igbe et al., Oncotarget, 2017, 8, 113734; and PCT applications: WO2015107493; WO2015107494; WO201507495; WO2016203404; WO2016203405; WO2016203406; WO2011022440; WO2017156397; WO2017079723; WO2017211303; WO2012041524; WO2017211303; WO2019051084; WO2017211303; US20160030594; US20110281942; WO2010011666; WO2014113584; WO2014176488; WO2017100279; WO2019051469; U.S. Pat. No. 8,637,684; WO2007117699; WO2015003094; WO2005094314; WO2008124815; WO2009049098; WO2009135000; WO2016191328; WO2016196591; WO2017078499; WO2017210134; WO2018013597; WO2018129402; WO2018130928; WO20181309928; WO2018136264; WO2018136265; WO2018160731; WO2018172984; and WO2010121212, each of which is incorporated herein by reference.

In some embodiments, a SHP2 inhibitor binds in the active site. In some embodiments, a SHP2 inhibitor is a mixed-type irreversible inhibitor. In some embodiments, a SHP2 inhibitor binds an allosteric site e.g., a non-covalent allosteric inhibitor. In some embodiments, a SHP2 inhibitor is a covalent SHP2 inhibitor, such as an inhibitor that targets the cysteine residue (C333) that lies outside the phosphatase's active site. In some embodiments a SHP2 inhibitor is a reversible inhibitor. In some embodiments, a SHP2 inhibitor is an irreversible inhibitor. In some embodiments, the SHP2 inhibitor is SHP099. In some embodiments, the SHP2 inhibitor is TNO155. In some embodiments, the SHP2 inhibitor is RMC-4550. In some embodiments, the SHP2 inhibitor is RCM-4630. In some embodiments, the SHP2 inhibitor is JAB-3068.

Proteasome inhibitors include, but are not limited to, carfilzomib (Kyprolis®), bortezomib (Velcade®), and oprozomib.

Immune therapies include, but are not limited to, monoclonal antibodies, immunomodulatory imides (IMiDs), GITR agonists, genetically engineered T-cells (e.g., CAR-T cells), bispecific antibodies (e.g., BiTEs), and anti-PD-1, anti-PDL-1, anti-CTLA4, anti-LAGl, and anti-OX40 agents).

Immunomodulatory agents (IMiDs) are a class of immunomodulatory drugs (drugs that adjust immune responses) containing an imide group. The IMiD class includes thalidomide and its analogues (lenalidomide, pomalidomide, and apremilast).

Exemplary anti-PD-1 antibodies and methods for their use are described by Goldberg et al., Blood 2007, 110(1):186-192; Thompson et al., Clin. Cancer Res. 2007, 13(6):1757-1761; and WO06/121168 A1), as well as described elsewhere herein.

GITR agonists include, but are not limited to, GITR fusion proteins and anti-GITR antibodies (e.g., bivalent anti-GITR antibodies), such as, a GITR fusion protein described in U.S. Pat. Nos. 6,111,090, 8,586,023, WO2010/003118 and WO2011/090754; or an anti-GITR antibody described, e.g., in U.S. Pat. No. 7,025,962, EP 1947183, U.S. Pat. Nos. 7,812,135, 8,388,967, 8,591,886, 7,618,632, EP 1866339, and WO2011/028683, WO2013/039954, WO05/007190, WO07/133822, WO05/055808, WO99/40196, WO01/03720, WO99/20758, WO06/083289, WO05/115451, and WO2011/051726.

Another example of a therapeutic agent that may be used in combination with the compounds of the invention is an anti-angiogenic agent. Anti-angiogenic agents are inclusive of, but not limited to, in vitro synthetically prepared chemical compositions, antibodies, antigen binding regions, radionuclides, and combinations and conjugates thereof. An anti-angiogenic agent can be an agonist, antagonist, allosteric modulator, toxin or, more generally, may act to inhibit or stimulate its target (e.g., receptor or enzyme activation or inhibition), and thereby promote cell death or arrest cell growth. In some embodiments, the one or more additional therapies include an anti-angiogenic agent.

Anti-angiogenic agents can be MMP-2 (matrix-metalloproteinase 2) inhibitors, MMP-9 (matrix-metalloprotienase 9) inhibitors, and COX-II (cyclooxygenase 11) inhibitors. Non-limiting examples of anti-angiogenic agents include rapamycin, temsirolimus (CCI-779), everolimus (RAD001), sorafenib, sunitinib, and bevacizumab. Examples of useful COX-II inhibitors include alecoxib, valdecoxib, and rofecoxib. Examples of useful matrix metalloproteinase inhibitors are described in WO96/33172, WO96/27583, WO98/07697, WO98/03516, WO98/34918, WO98/34915, WO98/33768, WO98/30566, WO90/05719, WO99/52910, WO99/52889, WO99/29667, WO99007675, EP0606046, EP0780386, EP1786785, EP1181017, EP0818442, EP1004578, and US20090012085, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 or AMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13). Some specific examples of MMP inhibitors are AG-3340, RO 32-3555, and RS 13-0830.

Further exemplary anti-angiogenic agents include KDR (kinase domain receptor) inhibitory agents (e.g., antibodies and antigen binding regions that specifically bind to the kinase domain receptor), anti-VEGF agents (e.g., antibodies or antigen binding regions that specifically bind VEGF, or soluble VEGF receptors or a ligand binding region thereof) such as VEGF-TRAP™, and anti-VEGF receptor agents (e.g., antibodies or antigen binding regions that specifically bind thereto), EGFR inhibitory agents (e.g., antibodies or antigen binding regions that specifically bind thereto) such as Vectibix® (panitumumab), erlotinib (Tarceva®), anti-Angl and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto). Other anti-angiogenic agents include Campath, IL-8, B-FGF, Tek antagonists (US2003/0162712; U.S. Pat. No. 6,413,932), anti-TWEAK agents (e.g., specifically binding antibodies or antigen binding regions, or soluble TWEAK receptor antagonists; see U.S. Pat. No. 6,727,225), ADAM distintegrin domain to antagonize the binding of integrin to its ligands (US 2002/0042368), specifically binding anti-eph receptor or anti-ephrin antibodies or antigen binding regions (U.S. Pat. Nos. 5,981,245; 5,728,813; 5,969,110; 6,596,852; 6,232,447; 6,057,124 and patent family members thereof, and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto). Additional anti-angiogenic agents include: SD-7784 (Pfizer, USA); cilengitide (Merck KGaA, Germany, EPO 0770622); pegaptanib octasodium, (Gilead Sciences, USA); Alphastatin, (BioActa, UK); M-PGA, (Celgene, USA, U.S. Pat. No. 5,712,291); ilomastat, (Arriva, USA, U.S. Pat. No. 5,892,112); emaxanib, (Pfizer, USA, U.S. Pat. No. 5,792,783); vatalanib, (Novartis, Switzerland); 2-methoxyestradiol (EntreMed, USA); TLC ELL-12 (Elan, Ireland); anecortave acetate (Alcon, USA); alpha-D148 Mab (Amgen, USA); CEP-7055 (Cephalon, USA); anti-Vn Mab (Crucell, Netherlands), DACantiangiogenic (ConjuChem, Canada); Angiocidin (InKine Pharmaceutical, USA); KM-2550 (Kyowa Hakko, Japan); SU-0879 (Pfizer, USA); CGP-79787 (Novartis, Switzerland, EP 0970070); ARGENT technology (Ariad, USA); YIGSR-Stealth (Johnson & Johnson, USA); fibrinogen-E fragment (BioActa, UK); angiogenic inhibitor (Trigen, UK); TBC-1635 (Encysive Pharmaceuticals, USA); SC-236 (Pfizer, USA); ABT-567 (Abbott, USA); Metastatin (EntreMed, USA); maspin (Sosei, Japan); 2-methoxyestradiol (Oncology Sciences Corporation, USA); ER-68203-00 (IV AX, USA); BeneFin (Lane Labs, USA); Tz-93 (Tsumura, Japan); TAN-1120 (Takeda, Japan); FR-111142 (Fujisawa, Japan, JP 02233610); platelet factor 4 (RepliGen, USA, EP 407122); vascular endothelial growth factor antagonist (Borean, Denmark); bevacizumab (pINN) (Genentech, USA); angiogenic inhibitors (SUGEN, USA); XL 784 (Exelixis, USA); XL 647 (Exelixis, USA); MAb, alpha5beta3 integrin, second generation (Applied Molecular Evolution, USA and Medlmmune, USA); enzastaurin hydrochloride (Lilly, USA); CEP 7055 (Cephalon, USA and Sanofi-Synthelabo, France); BC 1 (Genoa Institute of Cancer Research, Italy); rBPI 21 and BPI-derived antiangiogenic (XOMA, USA); PI 88 (Progen, Australia); cilengitide (Merck KGaA, German; Munich Technical University, Germany, Scripps Clinic and Research Foundation, USA); AVE 8062 (Ajinomoto, Japan); AS 1404 (Cancer Research Laboratory, New Zealand); SG 292, (Telios, USA); Endostatin (Boston Childrens Hospital, USA); ATN 161 (Attenuon, USA); 2-methoxyestradiol (Boston Childrens Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProlX, USA); METASTATIN, (EntreMed, USA); troponin I, (Harvard University, USA); SU 6668, (SUGEN, USA); OXI 4503, (OXiGENE, USA); o-guanidines, (Dimensional Pharmaceuticals, USA); motuporamine C, (British Columbia University, Canada); CDP 791, (Celltech Group, UK); atiprimod (pINN), (GlaxoSmithKline, UK); E 7820, (Eisai, Japan); CYC 381, (Harvard University, USA); AE 941, (Aeterna, Canada); vaccine, angiogenic, (EntreMed, USA); urokinase plasminogen activator inhibitor, (Dendreon, USA); oglufanide (pINN), (Melmotte, USA); HIF-lalfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol; anginex (Maastricht University, Netherlands, and Minnesota University, USA); ABT 510 (Abbott, USA); AAL 993 (Novartis, Switzerland); VEGI (ProteomTech, USA); tumor necrosis factor-alpha inhibitors; SU 11248 (Pfizer, USA and SUGEN USA); ABT 518, (Abbott, USA); YH16 (Yantai Rongchang, China); S-3APG (Boston Childrens Hospital, USA and EntreMed, USA); MAb, KDR (ImClone Systems, USA); MAb, alpha5 beta (Protein Design, USA); KDR kinase inhibitor (Celltech Group, UK, and Johnson & Johnson, USA); GFB 116 (South Florida University, USA and Yale University, USA); CS 706 (Sankyo, Japan); combretastatin A4 prodrug (Arizona State University, USA); chondroitinase AC (IBEX, Canada); BAY RES 2690 (Bayer, Germany); AGM 1470 (Harvard University, USA, Takeda, Japan, and TAP, USA); AG 13925 (Agouron, USA); Tetrathiomolybdate (University of Michigan, USA); GCS 100 (Wayne State University, USA) CV 247 (Ivy Medical, UK); CKD 732 (Chong Kun Dang, South Korea); irsogladine, (Nippon Shinyaku, Japan); RG 13577 (Aventis, France); WX 360 (Wilex, Germany); squalamine, (Genaera, USA); RPI 4610 (Sirna, USA); heparanase inhibitors (InSight, Israel); KL 3106 (Kolon, South Korea); Honokiol (Emory University, USA); ZK CDK (Schering AG, Germany); ZK Angio (Schering AG, Germany); ZK 229561 (Novartis, Switzerland, and Schering AG, Germany); XMP 300 (XOMA, USA); VGA 1102 (Taisho, Japan); VE-cadherin-2 antagonists(ImClone Systems, USA); Vasostatin (National Institutes of Health, USA); Flk-1 (ImClone Systems, USA); TZ 93 (Tsumura, Japan); TumStatin (Beth Israel Hospital, USA); truncated soluble FLT 1 (vascular endothelial growth factor receptor 1) (Merck & Co, USA); Tie-2 ligands (Regeneron, USA); and thrombospondin 1 inhibitor (Allegheny Health, Education and Research Foundation, USA).

Further examples of therapeutic agents that may be used in combination with compounds of the invention include agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor, c-Met.

Another example of a therapeutic agent that may be used in combination with compounds of the invention is an autophagy inhibitor. Autophagy inhibitors include, but are not limited to chloroquine, 3-methyladenine, hydroxychloroquine (Plaquenil™) bafilomycin A1, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside, and vinblastine. In addition, antisense or siRNA that inhibits expression of proteins including but not limited to ATG5 (which are implicated in autophagy), may also be used. In some embodiments, the one or more additional therapies include an autophagy inhibitor.

Another example of a therapeutic agent that may be used in combination with compounds of the invention is an anti-neoplastic agent. In some embodiments, the one or more additional therapies include an anti-neoplastic agent. Non-limiting examples of anti-neoplastic agents include acemannan, aclarubicin, aldesleukin, alemtuzumab, alitretinoin, altretamine, amifostine, aminolevulinic acid, amrubicin, amsacrine, anagrelide, anastrozole, ancer, ancestim, arglabin, arsenic trioxide, BAM-002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflomithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-Nl, interferon alfa-n3, interferon alfacon-1, interferon alpha, natural, interferon beta, interferon beta-1a, interferon beta-1b, interferon gamma, natural interferon gamma-1a, interferon gamma-1b, interleukin-1 beta, iobenguane, irinotecan, irsogladine, lanreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburiembodiment, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, virulizin, zinostatin stimalamer, or zoledronic acid; abarelix; AE 941 (Aeterna), ambamustine, antisense oligonucleotide, bcl-2 (Genta), APC 8015 (Dendreon), decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techni clone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

Additional examples of therapeutic agents that may be used in combination with compounds of the invention include ipilimumab (Yervoy®); tremelimumab; galiximab; nivolumab, also known as BMS-936558 (Opdivo®); pembrolizumab (Keytruda®); avelumab (Bavencio®); AMP224; BMS-936559; MPDL3280A, also known as RG7446; MEDI-570; AMG557; MGA271; IMP321; BMS-663513; PF-05082566; CDX-1127; anti-OX40 (Providence Health Services); huMAbOX40L; atacicept; CP-870893; lucatumumab; dacetuzumab; muromonab-CD3; ipilumumab; MEDI4736 (Imfinzi@); MSB0010718C; AMP 224; adalimumab (Humira®); ado-trastuzumab emtansine (Kadcyla®); aflibercept (Eylea®); alemtuzumab (Campath®); basiliximab (Simulect@); belimumab (Benlysta®); basiliximab (Simulect®); belimumab (Benlysta®); brentuximab vedotin (Adcetris®); canakinumab (Ilaris®); certolizumab pegol (Cimzia®); daclizumab (Zenapax®); daratumumab (Darzalex®); denosumab (Prolia®); eculizumab (Soliris®); efalizumab (Raptiva®); gemtuzumab ozogamicin (Mylotarg®); golimumab (Simponi®); ibritumomab tiuxetan (Zevalin®); infliximab (Remicade®); motavizumab (Numax®); natalizumab (Tysabri®); obinutuzumab (Gazyva®); ofatumumab (Arzerra®); omalizumab (Xolair®); palivizumab (Synagis®); pertuzumab (Perjeta®); pertuzumab (Perjeta®); ranibizumab (Lucentis®); raxibacumab (Abthrax®); tocilizumab (Actemra®); tositumomab; tositumomab-i-131; tositumomab and tositumomab-i-131 (Bexxar®); ustekinumab (Stelara®); AMG 102; AMG 386; AMG 479; AMG 655; AMG 706; AMG 745; and AMG 951.

In some embodiments, an additional compound used in combination therapy with a compound of the present invention is selected from the group consisting of a CDK4/6 inhibitor (e.g., abemaciclib, palbociclib, or ribociclib), a KRAS:GDP G12C inhibitor (e.g., AMG 510, MRTX 1257) or other mutant Ras:GDP inhibitor, a KRAS:GTP G12C inhibitor or other mutant Ras:GTP inhibitor, a MEK inhibitor (e.g., refametinib, selumetinib, trametinib, or cobimetinib), a SHP2 inhibitor (e.g., TNO155, RMC-4630), an ERK inhibitor, and an RTK inhibitor (e.g., an EGFR inhibitor).

In some embodiments, an additional compound used in combination therapy with a compound of the present invention is selected from the group consisting of ABT-737, AT-7519, carfilzomib, cobimetinib, danusertib, dasatinib, doxorubicin, GSK-343, JQ1, MLN-7243, NVP-ADW742, paclitaxel, palbociclib and volasertib. In some embodiments, an additional compound used in combination therapy with a compound of the present invention is selected from the group consisting of neratinib, acetinib and reversine.

The compounds described herein can be used in combination with the agents disclosed herein or other suitable agents, depending on the condition being treated. Hence, in some embodiments the one or more compounds of the disclosure will be co-administered with other therapies as described herein. When used in combination therapy, the compounds described herein may be administered with the second agent simultaneously or separately. This administration in combination can include simultaneous administration of the two agents in the same dosage form, simultaneous administration in separate dosage forms, and separate administration. That is, a compound described herein and any of the agents described herein can be formulated together in the same dosage form and administered simultaneously. Alternatively, a compound of the invention and any of the therapies described herein can be simultaneously administered, wherein both the agents are present in separate formulations. In another alternative, a compound of the present disclosure can be administered and followed by any of the therapies described herein, or vice versa. In some embodiments of the separate administration protocol, a compound of the invention and any of the therapies described herein are administered a few minutes apart, or a few hours apart, or a few days apart.

In some embodiments, a combination therapeutic regimen employs two therapeutic agents, one compound of the present invention and a second selected from the therapeutic agents described herein. In some embodiments, a combination therapeutic regimen employs three therapeutic agents, one compound of the present invention and two selected from the therapeutic agents described herein. In some embodiments, a combination therapeutic regimen employs four or more therapeutic agents, one compound of the present invention and three selected from the therapeutic agents described herein.

In some embodiments of any of the methods described herein, the first therapy (e.g., a compound of the invention) and one or more additional therapies are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours, up to 24 hours, or up to 1-7, 1-14, 1-21 or 1-30 days before or after the one or more additional therapies.

The invention also features kits including (a) a pharmaceutical composition including an agent (e.g., a compound of the invention) described herein, and (b) a package insert with instructions to perform any of the methods described herein. In some embodiments, the kit includes (a) a pharmaceutical composition including an agent (e.g., a compound of the invention) described herein, (b) one or more additional therapies (e.g., non-drug treatment or therapeutic agent), and (c) a package insert with instructions to perform any of the methods described herein.

As one aspect of the present invention contemplates the treatment of the disease or symptoms associated therewith with a combination of pharmaceutically active compounds that may be administered separately, the invention further relates to combining separate pharmaceutical compositions in kit form. The kit may comprise two separate pharmaceutical compositions: a compound of the present invention, and one or more additional therapies. The kit may comprise a container for containing the separate compositions such as a divided bottle or a divided foil packet. Additional examples of containers include syringes, boxes, and bags. In some embodiments, the kit may comprise directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

In this Combination Therapy section, all references are incorporated by reference for the agents described, whether explicitly stated as such or not.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

Definitions used in the following examples and elsewhere herein are:

CH₂Cl₂, DCM Methylene chloride, Dichloromethane

ACN, CH₃CN, Acetonitrile

MeCN

DAST Diethylaminosulfur trifluoride

DCE Dichloroethane

DIEA N,N-diisopropylethylamine

DIPEA Diisopropylethyl amine

DMA Dimethylacetamide

DMF N,N-Dimethylformamide

EtOAc Ethyl acetate

h Hour

H₂O Water

HCl Hydrochloric acid

K₃PO₄ Potassium phosphate (tribasic)

MeOH Methanol

Na₂SO₄ Sodium sulfate

NCS N-chlorosuccinimide

NMP N-methyl pyrrolidone

rt Room temperature

T3P Propanephosphonic acid anhydride

TBAF Tetrabutylammonium fluoride

TEA Triethylamine

TFA Trifluoroacetic acid

THF Tetrahydrofuran

TMSBr Trimethylsilyl bromide

TMSCF₃ Trifluoromethyltrimethylsilane

### Example 1. Synthesis of N-[(1R)-1-[3-amino-5-(trifluoromethyl)phenyl]ethyl]-2-chloro-6-(morpholine-4-carbonyl)-5H,6H,7H-pyrrolo[3,4-d]pyrimidin-4-amine

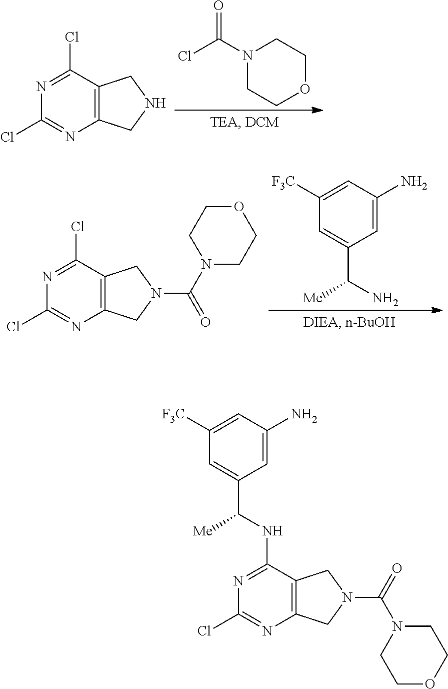 [SMILES: Clc1nc(Cl)c2c(n1)CNC2 ; O=C(Cl)N1CCOCC1 ; O=C(N1CCOCC1)N1Cc2nc(Cl)nc(Cl)c2C1 ; C[C@@H](N)c1cc(N)cc(C(F)(F)F)c1 ; C[C@@H](Nc1nc(Cl)nc2c1CN(C(=O)N1CCOCC1)C2)c1cc(N)cc(C(F)(F)F)c1]

### Step 1.

A mixture of 2,4-dichloro-6,7-dihydro-5H-pyrrolo[3,4-d]pyrimidine (105 mg, 551 μmol) and TEA (230 μL, 1.65 mmol) in DCM (1 mL) was added morpholine-4-carbonyl chloride (64 μL, 551.44 μmol) the mixture was stirred at 25° C. for 1 h and then concentrated under reduced pressure. The crude residue was purified by prep-TLC to give (2,4-dichloro-5,7-dihydropyrrolo[3,4-d]pyrimidin-6-yl)-morpholino-methanone (20 mg, 12% yield). LCMS (ESI): m/z: [M+H] calculated for C₁₁H₁₃Cl₂N₄O₂: 303.0. found 303.1.

### Step 2.

To a mixture of (2,4-dichloro-5,7-dihydropyrrolo[3,4-d]pyrimidin-6-yl)-morpholino-methanone (20 mg, 66 μmol) and 3-[(1R)-1-aminoethyl]-5-(trifluoromethyl)aniline hydrochloride (21 mg, 86 μmol) in n-BuOH (1 mL) was added DIEA (114.92 μL, 660 μmol). The mixture was stirred at 100° C. for 2 h, cooled to rt and filtered. The solvent was removed under reduced pressure and the crude product was purified by prep-HPLC to give [4-[[(1R)-1-[3-amino-5-(trifluoromethyl)phenyl]ethyl]amino]-2-chloro-5,7-dihydropyrrolo[3,4-d]pyrimidin-6-yl]-morpholino-methanone (6 mg, 19% yield). LCMS (ESI): m/z: [M+H] calculated for C₂₀H₂₃ClF₃N₆O₂: 471.1. found 471.0; ¹H NMR (400 MHz, METHANOL-d4) δ ppm 6.91 (d, J=9.54 Hz, 2H) 6.81 (s, 1H) 5.29-5.38 (m, 1H) 4.54-4.65 (m, 4H) 3.68-3.76 (m, 4H) 3.33-3.36 (m, 4H) 1.54 (d, J=7.09 Hz 3H,).

### Example 2. Synthesis of N-[(1R)-1-[3-amino-5-(trifluoromethyl)phenyl]ethyl]-2-methyl-6-(morpholine-4-carbonyl)-5H,6H,7H,8H-pyrido[4,3-d]pyrimidin-4-amine

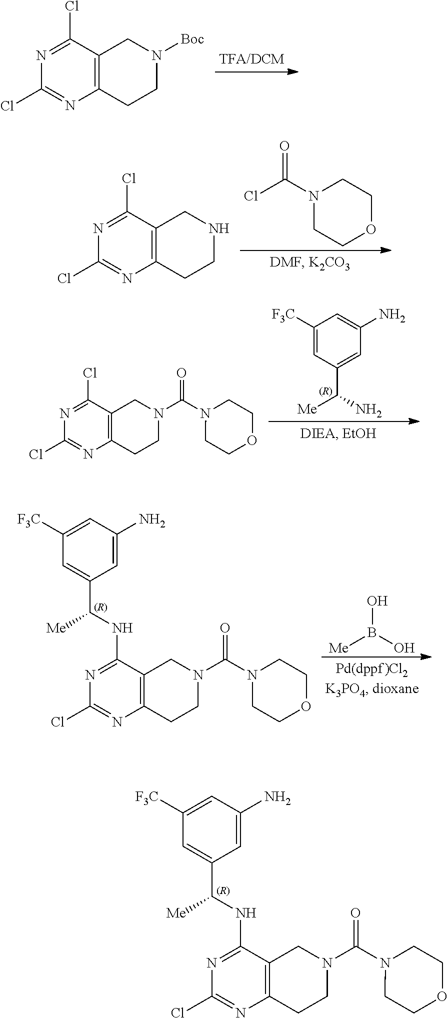

### Step 1.

A mixture of tert-butyl 2,4-dichloro-7,8-dihydro-5H-pyrido[4,3-d]pyrimidine-6-carboxylate (0.2 g, 660 μmol) in DCM (3 mL) was added TFA (1 mL) at 20° C. The mixture was stirred at 20° C. for 12 h and then concentrated to give 2,4-dichloro-5,6,7,8-tetrahydropyrido[4,3-d]pyrimidine (0.21 g) as yellow solid, which was used in the next step without further purification. ¹H NMR (400 MHz, METHANOL-d₄) δ ppm 4.41 (s, 2H) 3.62 (t, J=6.28 Hz, 2H) 3.21 (t, J=6.28 Hz, 2H).

### Step 2.

To a mixture of 2,4-dichloro-5,6,7,8-tetrahy dropyrido[4,3-d]pyrimidine trifluoroacetate (0.21 g, 660 μmol) in THF (5 mL) was added morpholine-4-carbonyl chloride (77 μL, 660 μmol) and TEA (460 μL, 3.3 mmol). The mixture was stirred at 20° C. for 1 h and then concentrated under reduced pressure. The crude residue was purified by silica gel chromatography to give (2,4-dichloro-7,8-dihydro-5H-pyrido[4,3-d]pyrimidin-6-yl)-morpholino-methanone (0.2 g, 95% yield). ¹H NMR (400 MHz, METHANOL-d₄) δ ppm 4.42 (s, 2H) 3.66-3.74 (m, 4H) 3.60 (t, J=5.84 Hz, 2H) 3.33-3.38 (m, 4H) 3.01 (t, J=5.84 Hz, 2H).

### Step 3.

To a mixture of (2,4-dichloro-7,8-dihydro-5H-pyrido[4,3-d]pyrimidin-6-yl)-morpholino-methanone (0.2 g, 630 μmol) in butan-1-ol (4 mL) was added 3-[(1R)-1-aminoethyl]-5-(trifluoromethyl)aniline hydrochloride (182 mg, 757 μmol) and DIEA (1.1 mL, 6.31 mmol). The mixture was stirred at 90° C. for 3 h and then concentrated under reduced pressure. The crude residue was purified by silica gel chromatography to give [4-[[(1R)-1-[3-amino-5-(trifluoromethyl)phenyl]ethyl]amino]-2-chloro-7,8-dihydro-5H-pyrido[4,3-d]pyrimidin-6-yl]-morpholino-methanone (0.28 g, 71% yield). ¹H NMR (400 MHz, METHANOL-d₄) δ ppm 6.94 (s, 1H) 6.91 (s, 1H) 6.81 (s, 1H) 5.39 (q, J=7.06 Hz, 1H) 4.16 (s, 2H) 3.68-3.71 (m, 4H) 3.53 (t, J=5.84 Hz, 2H) 3.32-3.37 (m, 4H) 2.75 (s, 2H) 1.56 (d, J=7.06 Hz, 3H).

### Step 4.

To a solution of [4-[[(1R)-1-[3-amino-5-(trifluoromethyl)phenyl]ethyl]amino]-2-chloro-7,8-dihydro-5H-pyrido[4,3-d]pyrimidin-6-yl]-morpholino-methanone (0.28 g, 577 μmol) in DME (3 mL) was added methylboronic acid (242 mg, 4.0 mmol), H₂O (0.6 mL), Na₂CO₃ (367 mg, 3.5 mmol) and Pd(PPh₃)₄([1,1′-Bis(diphenylphosphino)ferrocene]dichloropalladium(II), 133 mg, 115 μmol). The reaction was stirred at 100° C. for 16 h, cooled to room temperature and concentrated under reduced pressure. The crude residue was purified by prep-HPLC to give [4-[[(1R)-1-[3-amino-5-(trifluoromethyl)phenyl]ethyl]amino]-2-methyl-7,8-dihydro-5H-pyrido[4,3-d]pyrimidin-6-yl]-morpholino-methanone (6 mg, 2% yield). ¹H NMR (400 MHz, METHANOL-d₄) δ ppm 6.93 (s, 2H) 6.78 (s, 1H) 5.43-5.50 (m, 1H) 4.16 (s, 2H) 3.65-3.76 (m, 4H) 3.54 (t, J=5.95 Hz, 2H) 3.33 (br s, 4H) 3.31-3.35 (m, 1H) 2.74 (t, J=5.62 Hz, 2H) 2.33 (s, 3H) 1.54 (d, J=7.06 Hz, 3H); LCMS (ESI): m/z: [M+H] calculated for C₂₂H₂₈F₃N₆O₂: 465.2. found 465.2.

### Example 3. Synthesis of N-[(1R)-1-[3-amino-5-(trifluoromethyl)phenyl]ethyl]-2-chloro-6-(morpholine-4-carbonyl)-5H,6H,7H,8H-pyrido[4,3-d]pyrimidin-4-amine

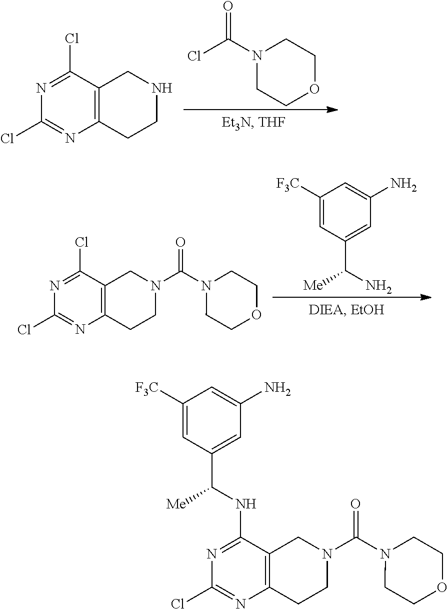 [SMILES: Clc1nc(Cl)c2c(n1)CCNC2 ; O=C(Cl)N1CCOCC1 ; O=C(N1CCOCC1)N1CCc2nc(Cl)nc(Cl)c2C1 ; C[C@@H](N)c1cc(N)cc(C(F)(F)F)c1 ; C[C@@H](Nc1nc(Cl)nc2c1CN(C(=O)N1CCOCC1)CC2)c1cc(N)cc(C(F)(F)F)c1]

To a solution of 2,4-dichloro-5,6,7,8-tetrahy dropyrido[4,3-d]pyrimidine (200 mg, 980 μmol) and morpholine-4-carbonyl chloride (137 μL, 1.18 mmol) in THF (5 mL) was added TEA (409 μL, 2.94 mmol). The mixture was stirred at 0° C. for 0.5 h, and then warmed to 25° C. for 2 h. Water (20 mL) was added and the mixture was extracted with EtOAc. The combined organic phases were washed with brine and dried over Na₂SO₄. The solvent was removed under reduced pressure and the crude residue was purified by prep-TLC to give (2,4-dichloro-7,8-dihydro-5H-pyrido[4,3-d]pyrimidin-6-yl)-morpholino-methanone (150 mg, 48% yield). ¹H NMR (400 MHz, DMSO-d₆) δ ppm 4.30 (s, 2H) 3.61-3.55 (m, 4H) 3.49 (t, J=5.75 Hz, 2H) 3.25-3.19 (m, 4H) 2.96 (t, J=5.69 Hz, 2H).

### Step 2.

To a solution of (2,4-dichloro-7,8-dihydro-5H-pyrido[4,3-d]pyrimidin-6-yl)-morpholino-methanone (50 mg, 157 μmol) and 3-[(1R)-1-aminoethyl]-5-(trifluoromethyl)aniline (41 mg, 205 μmol) in EtOH (2 mL) was added DIEA (82 μL, 473 μmol). The mixture was stirred at 100° C. for 3 h, cooled to rt and the solvent was removed under reduced pressure. The crude residue was purified by prep-HPLC to give [4-[[(1R)-1-[3-amino-5-(trifluoromethyl)phenyl]ethyl]amino]-2-chloro-7,8-dihydro-SH-pyrido[4,3-d]pyrimidin-6-yl]-morpholino-methanone (15 mg, 19% yield). LCMS (ESI): m/z: [M+H] calculated for C₂₁H₂₅ClF₃N₆O₂: 485.2. found 485.2; H NMR (400 MHz, METHANOL-d₄) δ ppm 6.94 (s, 1H) 6.91 (s, 1H) 6.81 (s, 1H) 5.39 (q, J=7.05 Hz, 1H) 4.16 (s, 2H) 3.71-3.66 (m, 4H) 3.53 (t, J=5.81 Hz, 2H) 3.35-3.32 (m, 4H) 2.75 (t, J=5.62 Hz, 2H) 1.56 (d, J=6.97 Hz, 3H).

### Example 4. Synthesis of N-[(1R)-1-[3-amino-5-(trifluoromethyl)phenyl]ethyl]-2-methyl-6-(morpholine-4-carbonyl)-5H,6H,7H-pyrrolo[3,4-d]pyrimidin-4-amine

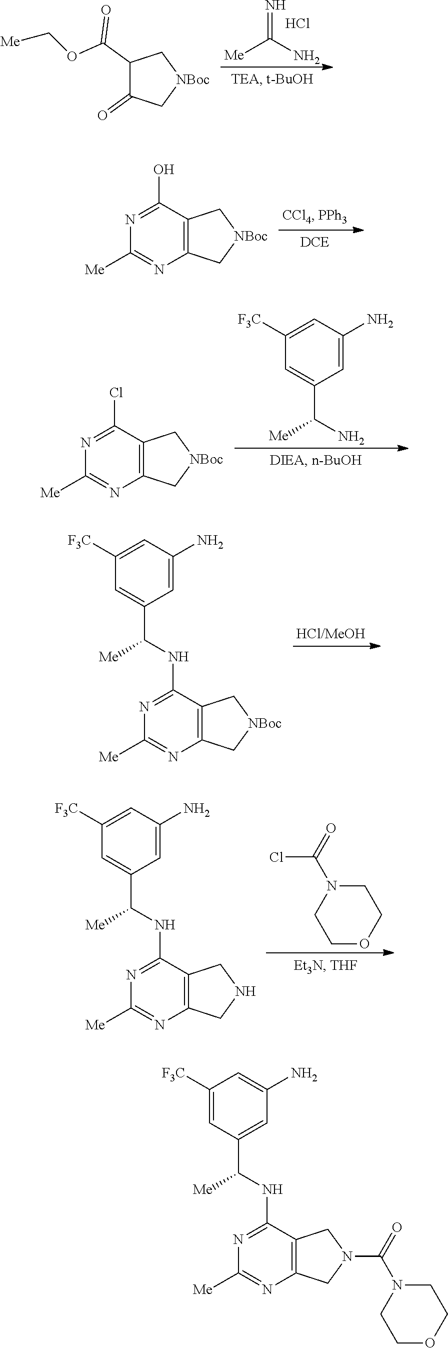

### Step 1.

To a mixture of O1-tert-butyl O3-ethyl 4-oxopyrrolidine-1,3-dicarboxylate (4 g, 15.6 mmol) and acetamidine hydrochloride (1.47 g, 15.6 mmol) in t-BuOH (40 mL) was added TEA (5.6 mL, 40.4 mmol). The mixture was stirred at 90° C. for 1 h, cooled to rt and the solvent was removed under reduced pressure. The crude product was purified by reversed-phase column to give tert-butyl 4-hydroxy-2-methyl-5,7-dihydropyrrolo[3,4-d]pyrimidine-6-carboxylate (1 g, 25% yield). LCMS (ESI): m/z: [M+H] calculated for C₁₂H₁₈N₃O₃: 252.1. found; 252.3.

### Step 2.

To a mixture of tert-butyl 4-hydroxy-2-methyl-5,7-dihydropyrrolo[3,4-d]pyrimidine-6-carboxylate (440 mg, 1.75 mmol) and PPh₃ (918 mg, 3.5 mmol) in DCE (4 mL) was added CCl₄ (505 μL, 5.25 mmol). The mixture was stirred at 70° C. for 3 h. The reaction was cooled to rt and the solvent was removed under reduced pressure to give a crude residue, which was combined with another batch (80 mmol starting material) for purification by column chromatography to give tert-butyl 4-chloro-2-methyl-5,7-dihydropyrrolo[3,4-d]pyrimidine-6-carboxylate (460 mg, 67% yield). ¹H NMR (400 MHz, METHANOL-d₄) δ=4.73-4.59 (m, 4H), 2.71-2.61 (m, 3H), 1.58-1.48 (m, 9H).

### Step 3.

To a mixture of tert-butyl 4-chloro-2-methyl-5,7-dihydropyrrolo[3,4-d]pyrimidine-6-carboxylate (200 mg, 741 μmol) and 3-[(1R)-1-aminoethyl]-5-(trifluoromethyl)aniline (196 mg, 964 μmol) in n-BuOH (2 mL) was added DIEA (387 μL, 2.2 mmol). The mixture was stirred at 110° C. for 12 h, cooled to rt and the solvent was removed under reduced pressure. The crude residue was purified by column chromatography to give tert-butyl 4-[[(1R)-1-[3-amino-5-(trifluoromethyl)phenyl]ethyl]amino]-2-methyl-5,7-dihydropyrrolo[3,4-d]pyrimidine-6-carboxylate (250 mg, 77% yield). ¹H NMR (400 MHz, METHANOL-d₄) δ=6.96-6.87 (m, 2H), 6.81-6.76 (m, 1H), 5.46-5.34 (m, 1H), 4.54-4.40 (m, 4H), 2.42-2.35 (m, 3H), 2.01 (s, 2H), 1.52 (d, J=2.0 Hz, 10H).

### Step 4.

tert-Butyl 4-[[(1R)-1-[3-amino-5-(trifluoromethyl)phenyl]ethyl]amino]-2-methyl-5,7-dihydropyrrolo[3,4-d]pyrimidine-6-carboxylate (140 mg, 320 μmol) was dissolved in HCl/MeOH (3 mL). After stirring at rt for 1 h the solvent was removed under reduced pressure to give N-[(1R)-1-[3-amino-5-(trifluoromethyl)phenyl]ethyl]-2-methyl-6,7-dihydro-5H-pyrrolo[3,4-d]pyrimidin-4-amine hydrochloride (119 mg, 99% yield). ¹H NMR (400 MHz, METHANOL-d₄) δ=7.91-7.81 (m, 2H), 7.62-7.58 (m, 1H), 5.72-5.64 (m, 1H), 4.76-4.62 (m, 4H), 2.59-2.54 (m, 3H), 1.73-1.64 (m, 3H).

### Step 5.

To a mixture of N-[(1R)-1-[3-amino-5-(trifluoromethyl)phenyl]ethyl]-2-methyl-6,7-dihydro-5H-pyrrolo[3,4-d]pyrimidin-4-amine hydrochloride (110 mg, 294.27 μmol) in THF (2 mL) was added TEA (163 μL, 1.18 mmol) and morpholine-4-carbonyl chloride (34 μL, 294 μmol) at 0° C. The mixture was stirred at 25° C. for 30 min, the solvent was removed under reduced pressure and the crude residue was purified by prep-HPLC to give [4-[[(1R)-1-[3-amino-5-(trifluoromethyl)phenyl]ethyl]amino]-2-methyl-5,7-dihydropyrrolo[3,4-d]pyrimidin-6-yl]-morpholino-methanone formate (41 mg, 27% yield). LCMS (ESI): m/z: [M+H] calculated for C₂₁H₂₆F₃N₆O₂: 450.3. found; 450.3; H NMR (400 MHz, METHANOL-d₄) δ=8.21 (s, 1H), 6.96-6.87 (m, 2H), 6.84-6.78 (m, 1H), 5.46-5.36 (m, 1H), 4.60 (d, J=17.0 Hz, 4H), 3.75-3.71 (m, 4H), 3.38-3.35 (m, 4H), 2.40 (s, 3H), 1.53 (d, J=6.8 Hz, 3H).

### Example 5. Synthesis of N-[(1R)-1-[3-amino-5-(trifluoromethyl)phenyl]ethyl]-2-chloro-6-(oxolane-3-carbonyl)-5H,6H,7H-pyrrolo[3,4-d]pyrimidin-4-amine

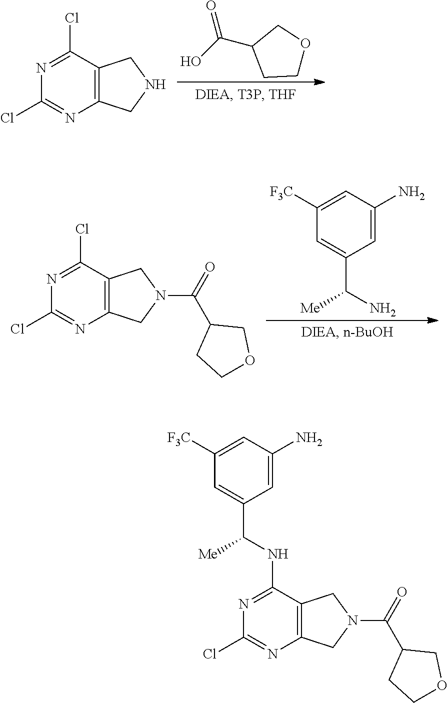 [SMILES: Clc1nc(Cl)c2c(n1)CNC2 ; O=C(O)C1CCOC1 ; O=C(C1CCOC1)N1Cc2nc(Cl)nc(Cl)c2C1 ; C[C@@H](N)c1cc(N)cc(C(F)(F)F)c1 ; C[C@@H](Nc1nc(Cl)nc2c1CN(C(=O)C1CCOC1)C2)c1cc(N)cc(C(F)(F)F)c1]

### Step 1.

To a solution of tetrahydrofuran-3-carboxylic acid (20 μL, 205 μmol) 2,4-dichloro-6,7-dihydro-5H-pyrrolo[3,4-d]pyrimidine trifluoroacetate (52 mg, 171 μmol) in THF (1 mL) was added DIPEA (89 μL, 513 μmol) and T3P (76 μL, 257 μmol). The mixture was stirred at 20° C. for 0.5 h. The solvent was removed under reduced pressure and the residue was purified by column chromatography to give (4,6-dichloro-1,3-dihydropyrrolo[3,4-c]pyridin-2-yl)-tetrahydrofuran-3-yl-methanone (20 mg, 70 μmol) as white solid. ¹H NMR (400 MHz, CHLOROFORM-d) δ ppm 4.89-4.98 (m, 2H) 4.85 (d, J=13.94 Hz, 2H) 4.06-4.15 (m, 1H) 3.96-4.03 (m, 4H) 3.87-3.95 (m, 4H) 3.78-3.87 (m, 2H) 3.18-3.29 (m, 1H) 3.07-3.17 (m, 2H) 2.10-2.32 (m, 6H).

### Step 2.

To a solution of (2,4-dichloro-5,7-dihydropyrrolo[3,4-d]pyrimidin-6-yl)-tetrahydrofuran-3-yl-methanone (20 mg, 70 μmol) and 3-[(1R)-1-aminoethyl]-5-(trifluoromethyl)aniline (18 mg, 90 μmol) in n-BuOH (1 mL) was added DIEA (121 μL, 694 μmol). The mixture was stirred at 100° C. for 2 h, cooled to rt and the solvent was removed under reduced pressure. The crude residue was purified by prep-HPLC to give [4-[[(1R)-1-[3-amino-5-(trifluoromethyl)phenyl]ethyl]amino]-2-chloro-5,7-dihydropyrrolo[3,4-d]pyrimidin-6-yl]-tetrahydrofuran-3-yl-methanone (6 mg, 19% yield). LCMS (ESI): m/z: [M+H] calculated for C₂₀H₂₁ClF₃N₅O₂: 455.9. found 456.1; ¹H NMR (400 MHz, DMSO-d₆) δ ppm 7.97-8.02 (m, 1H) 7.84-7.88 (m, 1H) 6.80-6.88 (m, 2H) 6.72-6.78 (m, 1H) 5.31-5.40 (m, 2H) 5.16-5.28 (m, 1H) 4.69 (dd, J=4.41, 1.54 Hz, 2H) 4.40-4.53 (m, 2H) 3.91-4.00 (m, 1H) 3.69-3.82 (m, 3H) 3.19-3.31 (m, 1H) 2.16 (d, J=7.28 Hz, 2H) 1.44-1.52 (m, 3H).

### Example 6. Synthesis of N-[(1R)-1-[3-amino-5-(trifluoromethyl)phenyl]ethyl]-2-methyl-6-(oxolane-3-carbonyl)-5H,6H,7H,8H-pyrido[4,3-d]pyrimidin-4-amine

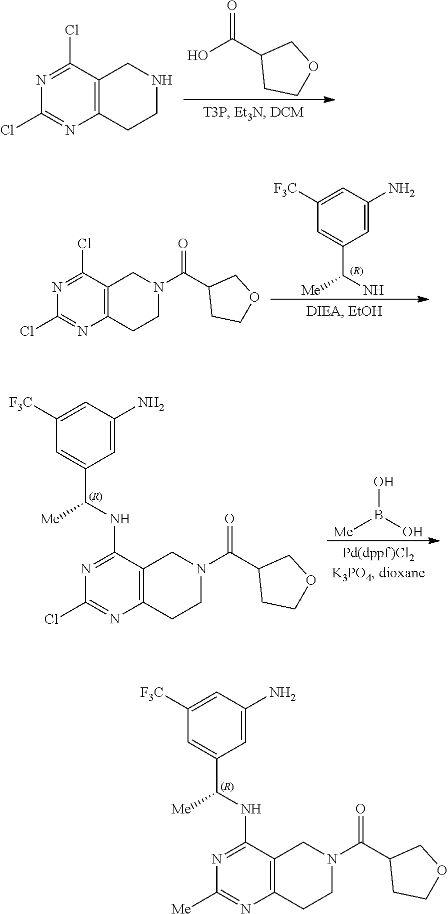

### Step 1.

To a mixture of tetrahydrofuran-3-carboxylic acid (76 μL, 792 μmol) and 2,4-dichloro-5,6,7,8-tetrahydropyrido[4,3-d]pyrimidine trifluoroacetate (0.21 g, 660 μmol) in THF (5 mL) was added DIEA (345 μL, 1.98 mmol), T₃P (295 μL, 990 μmol). The mixture was stirred at 25° C. for 0.5 h and then concentrated under reduced pressure. The crude residue was purified by silica gel chromatography to give (2,4-dichloro-7,8-dihydro-5H-pyrido[4,3-d]pyrimidin-6-yl)-tetrahydrofuran-3-yl-methanone (0.1 g, 50% yield) as white solid. ¹H NMR (400 MHz, METHANOL-d₄) δ ppm 4.69-4.79 (m, 2H) 3.78-4.03 (m, 6H) 3.48-3.63 (m, 1H) 3.04 (t, J=5.87 Hz, 1H) 2.94 (t, J=5.75 Hz, 1H) 2.06-2.30 (m, 2H).

### Step 2.

To a solution of (2,4-dichloro-7,8-dihydro-5H-pyrido[4,3-d]pyrimidin-6-yl)-tetrahydrofuran-3-yl-methanone (0.1 g, 331 μmol) in butan-1-ol (1 mL) was added 3-[(1R)-1-aminoethyl]-5-(trifluoromethyl)aniline hydrochloride (88 mg, 364 μmol) and DIEA (576 uL, 3.31 mmol). The mixture was stirred at 90° C. for 4 h and then concentrated under reduced pressure. The crude residue was purified by prep-TLC to give [4-[[(1R)-1-[3-amino-5-(trifluoromethyl)phenyl]ethyl]amino]-2-chloro-7,8-dihydro-5H-pyrido[4,3-d]pyrimidin-6-yl]-tetrahydrofuran-3-yl-methanone (0.13 g, 84% yield).

### Step 3.

To a solution of [4-[[(1R)-1-[3-amino-5-(trifluoromethyl)phenyl]ethyl]amino]-2-chloro-7,8-dihydro-5H-pyrido[4,3-d]pyrimidin-6-yl]-tetrahy drofuran-3-yl-methanone (130 mg, 277 μmol) in dioxane (2 mL) was added methylboronic acid (99 mg, 1.7 mmol), H₂O (0.4 mL), K₃PO₄ (352 mg, 1.7 mmol) and Pd(dppf)Cl₂—CH₂Cl₂ (45 mg, 55 μmol). The mixture was stirred at 80° C. for 2 h under N₂ After cooling to rt, the solvent was removed under reduced pressure and the crude residue was purified by prep-HPLC to give [4-[[(1R)-1-[3-amino-5-(trifluoromethyl)phenyl]ethyl]amino]-2-methyl-7,8-dihydro-5H-pyrido[4,3-d]pyrimidin-6-yl]-tetrahydrofuran-3-yl-methanone (10 mg, 8% yield) as the formate salt. LCMS (ESI): m/z: [M+H] calculated for C₂₂H₂₇F₃N₅O₂: 450.2. found 450.2; ¹H NMR (400 MHz, METHANOL-d₄) δ ppm 6.93 (br d, J=9.66 Hz, 2H) 6.79 (br s, 1H) 5.40-5.57 (m, 1H) 4.49 (d, J=5.01 Hz, 2H) 3.77-4.04 (m, 7H) 3.52-3.60 (m, 1H) 2.64-2.84 (m, 2H) 2.37 (s, 3H) 2.00-2.27 (m, 2H) 1.53-1.62 (m, 3H).

### Example 7. Synthesis of N-[(1R)-1-[3-amino-5-(trifluoromethyl)phenyl]ethyl]-2-chloro-7-(oxolane-3-carbonyl)-5H,6H,7H,8H,9H-pyrimido[4,5-d]azepin-4-amine

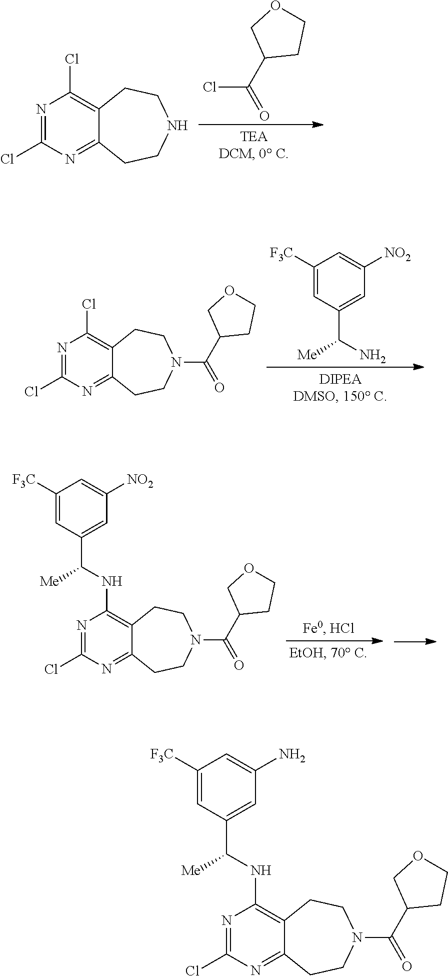 [SMILES: Clc1nc(Cl)c2c(n1)CCNCC2 ; O=C(Cl)C1CCOC1 ; O=C(C1CCOC1)N1CCc2nc(Cl)nc(Cl)c2CC1 ; C[C@@H](N)c1cc([N+](=O)[O-])cc(C(F)(F)F)c1 ; C[C@@H](Nc1nc(Cl)nc2c1CCN(C(=O)C1CCOC1)CC2)c1cc([N+](=O)[O-])cc(C(F)(F)F)c1 ; C[C@@H](Nc1nc(Cl)nc2c1CCN(C(=O)C1CCOC1)CC2)c1cc(N)cc(C(F)(F)F)c1]

### Step 1.

A solution of tetrahydrofuran-3-carbonyl chloride (106 mg, 0.79 mmol) in anhydrous DCM (0.4 mL) was added to a solution of 2,4-dichloro-5H,6H,7H,8H,9H-pyrimido[4,5-d]azepine hydrochloride (0.2 g, 0.79 mmol) in DCM (6 mL) and TEA (0.49 mL, 3.54 mmol) at 0° C. under an atmosphere of argon. After stirring for 12 h the reaction was diluted with DCM and washed with water, followed by brine. The combined organic phases were dried over Na₂SO₄, the solvent was removed under reduced pressure and the crude product was purified by flash chromatography to give 2,4-dichloro-7-(oxolane-3-carbonyl)-5H,6H,7H,8H,9H-pyrimido[4,5-d]azepine (122 mg, 49% yield). UPLC (ESI): m/z: [M+H] calculated for C₁₃H₁₅Cl₂N₃O₂: 316.2. found 316.5.

### Step 2.

A degassed mixture of 2,4-dichloro-7-(oxolane-3-carbonyl)-5H,6H,7H,8H,9H-pyrimido[4,5-d]azepine (122 mg, 0.39 mmol), DIPEA (269 μL, 1.54 mmol) and (1R)-1-[3-nitro-5-(trifluoromethyl)phenyl]ethan-1-amine hydrochloride (115 mg, 0.42 mmol) in anhydrous DMSO (3.5 mL) was stirred for 1 h at 150° C. in a microwave reactor. After cooling to rt the reaction was diluted with water and extracted with Et₂O. The combined organic phases were washed with water and dried over Na₂SO₄. The solvent was removed under reduced pressure and the crude product was purified by flash column chromatography to give 2-chloro-N-[(1R)-1-[3-nitro-5-(trifluoro-methyl)phenyl]ethyl]-7-(oxolane-3-carbonyl)-5H,6H,7H,8H,9H-pyrimido-[4,5-d]azepin-4-amin (130 mg, 66% yield). ¹H NMR (300 MHz, Chloroform-d) δ 8.44 (s, 1H), 8.39 (s, 1H), 8.03 (d, J=13.1 Hz, 1H), 5.51 (bs, 1H), 5.46-5.34 (m, 1H), 4.07-3.92 (m, 2H), 3.84 (dd, J=27.2, 10.9 Hz, 6H), 3.29-3.13 (m, 3H), 2.74 (s, 2H), 2.10 (dd, J=13.3, 6.0 Hz, 2H), 1.69 (d, J=6.9 Hz, 3H).

### Step 3.

To a solution of 2-chloro-N-[(1R)-1-[3-nitro-5-(trifluoromethyl)phenyl]ethyl]-7-(oxolane-3-carbonyl)-5H,6H,7H,8H,9H-pyrimido-[4,5-d]azepin-4-amine (130 mg, 0.25 mmol) in EtOH (2.5 mL) iron powder (78 mg, 1.39 mmol) and HCl_{aq} (1M, 1.0 mL, 1.0 mmol) were added. The mixture was stirred for 12 h at 70° C. The reaction mixture was filtered through Celite, washed with MeOH and the solvent was removed under reduced pressure. The crude product was purified by prep-HPLC to give N-[(1R)-1-[3-amino-5-(trifluoro-methyl)phenyl]ethyl]-2-chloro-7-(oxolane-3-carbonyl)-5H,6H,7H,8H,9H-pyrimido[4,5-d]azepin-4-amine (40 mg, 33% yield). LCMS (ESI): UPLC (ESI): m/z: [M+H] calculated for C₂₂H₂₅ClF₃N5O₂: 483.9. found 484.1; ¹H NMR (300 MHz, Methanol-d₄) δ 6.98-6.84 (m, 2H), 6.79 (s, 1H), 5.39-5.20 (m, 1H), 3.93 (q, J=7.9 Hz, 1H), 3.88-3.66 (m, 6H), 3.44 (p, J=7.9 Hz, 1H), 3.09-2.93 (m, 2H), 2.84 (dt, J=11.7, 6.4 Hz, 2H), 2.23-1.98 (m, 2H), 1.98-1.84 (m, 1H), 1.61-1.45 (m, 3H).

### Example 8. Synthesis of cis-4-(4-{[(1R)-1-[3-amino-5-(trifluoromethyl)phenyl]ethyl]amino}-2-chloro-5H,6H,7H-pyrrolo[3,4-d]pyrimidin-6-yl)cyclohexan-1-ol

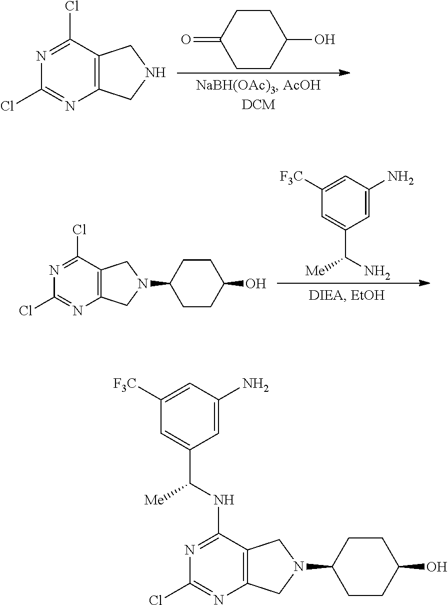 [SMILES: Clc1nc(Cl)c2c(n1)CNC2 ; O=C1CCC(O)CC1 ; O[C@H]1CC[C@@H](N2Cc3nc(Cl)nc(Cl)c3C2)CC1 ; C[C@@H](N)c1cc(N)cc(C(F)(F)F)c1 ; C[C@@H](Nc1nc(Cl)nc2c1CN([C@H]1CC[C@@H](O)CC1)C2)c1cc(N)cc(C(F)(F)F)c1]

### Step 1.

To a mixture of 2,4-dichloro-6,7-dihydro-5H-pyrrolo[3,4-d]pyrimidine hydrochloride (200 mg, 883 μmol) in DCM (1.4 mL) and AcOH (0.6 mL) was added NaBH(OAc)₃ (468 mg, 2.21 mmol) and 4-hydroxycyclohexanone (101 mg, 883 μmol). The mixture was stirred at 25° C. for 3 h and then filtered. The solvent was removed under reduced pressure and the crude residue was purified by prep-HPLC to give cis-4-(2,4-dichloro-5H-pyrrolo[3,4-d]pyrimidin-6(7H)-yl)cyclohexanol (60 mg, 208.21 μmol, 23.58% yield). LCMS (ESI): m/z: [M+H] calculated for C₁₂H₁₆Cl₂N₃O: 288.1. found 287.8.

### Step 2.

To a mixture of cis-4-(2,4-dichloro-5H-pyrrolo[3,4-d]pyrimidin-6(7H)-yl)cyclohexanol (58 mg, 201.27 μmol, 1 eq) in n-BuOH (1 mL) was added 3-[(1R)-1-aminoethyl]-5-(trifluoromethyl)aniline (53 mg, 262 μmol) and DIEA (351 μL, 2.0 mmol). The mixture was stirred at 100° C. for 8 h, cooled to rt and filtered. The solvent was removed under reduced pressure and the crude residue was purified by prep-HPLC to give cis-4-(4-(((R)-1-(3-amino-5-(trifluoromethyl)phenyl)ethyl)amino)-2-chloro-5H-pyrrolo[3,4-d]pyrimidin-6(7H)-yl)cyclohexanol (8 mg, 9% yield). LCMS (ESI): m/z: [M+H] calculated for C₂₁H₂₆ClF₃N₅O: 456.2. found 456.0; ¹H NMR (400 MHz, METHANOL-d4) δ ppm 6.90 (d, J=8.31 Hz, 2H) 6.80 (s, 1H) 5.26-5.35 (m, 1H) 3.84 (d, J=19.44 Hz, 5H) 2.50-2.59 (m, 1H) 1.68-1.84 (m, 6H) 1.56-1.65 (m, 2H) 1.52 (d, J=6.97 Hz, 3H).

### Example 9. Synthesis of trans-4-(4-{[(1R)-1-[3-amino-5-(trifluoromethyl)phenyl]ethyl]amino}-2-chloro-5H,6H,7H-pyrrolo[3,4-d]pyrimidin-6-yl)cyclohexan-1-ol

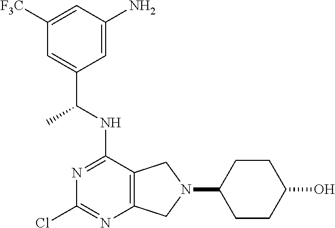

trans-4-(4-{[(1R)-1-[3-amino-5-(trifluoromethyl)phenyl]ethyl]amino}-2-chloro-5H,6H,7H-pyrrolo[3,4-d]pyrimidin-6-yl)cyclohexan-1-ol was synthesized in a manner similar to cis-4-(4-{[(1R)-1-[3-amino-5-(trifluoromethyl)phenyl]ethyl]amino}-2-chloro-5H,6H,7H-pyrrolo[3,4-d]pyrimidin-6-yl)cyclohexan-1-ol except cis-4-(2,4-dichloro-5H-pyrrolo[3,4-d]pyrimidin-6(7H)-yl)cyclohexanol was substituted with trans-4-(2,4-dichloro-5H-pyrrolo[3,4-d]pyrimidin-6(7H)-yl)cyclohexanol. LCMS (ESI): m/z: [M+H] calculated for C₂₁H₂₆ClF₃N₅O: 456.2. found 456.0; ¹H NMR (400 MHz, METHANOL-d4) δ ppm 6.89 (d, J=6.48 Hz, 2H,) 6.80 (s, 1H) 5.30 (d, J=5.99 Hz, 1H) 3.84 (d, J=18.58 Hz, 4H) 3.51-3.58 (m, 1H) 2.48 (s, 1H) 1.96-2.10 (m, 4H) 1.52 (d, J=7.09 Hz, 3H) 1.29-1.37 (m, 4H).

### Example 10. Synthesis of cis-4-(4-{[(1R)-1-[3-amino-5-(trifluoromethyl)phenyl]ethyl]amino}-2-chloro-5H,6H,7H-pyrrolo[3,4-d]pyrimidin-6-yl)-N,N-dimethylcyclohexane-1-carboxamide

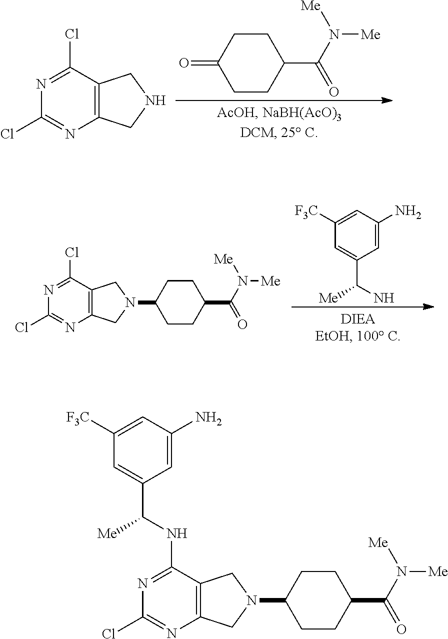

### Step 1.

To a mixture of 2,4-dichloro-6,7-dihydro-5H-pyrrolo[3,4-d]pyrimidine trifluoroacetate (1.68 g, 5.53 mmol) and N,N-dimethyl-4-oxo-cyclohexanecarboxamide (935 mg, 5.53 mmol) in DCM (11.2 mL) acetic acid (4.8 mL) and NaBH(OAc)₃ (4.10 g, 19.3 mmol) were added. The mixture was stirred at 25° C. for 3 h. The solvent was removed under reduced pressure and the residue was taken up in H₂O. The mixture was extracted with EtOAc and the combined organic phases were washed with brine and dried over NaSO₄. The solvent was removed under reduced pressure and the residue was purified by column chromatography to give cis-4-(2,4-dichloro-5,7-dihydropyrrolo[3,4-d]pyrimidin-6-yl)-N,N-dimethyl-cyclohexanecarboxamide (500 mg, 26% yield) and trans-4-(2,4-dichloro-5,7-dihydropyrrolo[3,4-d]pyrimidin-6-yl)-N,N-dimethyl-cyclohexanecarboxamide (150 mg, 8% yield. LCMS (ESI): m/z: [M+H] calculated for C₁₅H₂₁Cl₂N₄O: 343.10. found 343.2.

### Step 2.

To a solution of 3-[(1R)-1-aminoethyl]-5-(trifluoromethyl)aniline (93 mg, 454 μmol) and cis-4-(2,4-dichloro-5,7-dihydropyrrolo[3,4-d]pyrimidin-6-yl)-N,N-dimethyl-cyclohexanecarboxamide (120 mg, 350 μmol) in n-BuOH (1 mL) was added DIEA (609 μL, 3.5 mmol). The mixture was stirred at 100° C. for 5 h, cooled to rt and the solvent was removed under reduced pressure. The crude residue was purified by prep-HPLC to give cis-4-[4-[[(1R)-1-[3-amino-5-(trifluoromethyl)phenyl]ethyl]amino]-2-chloro-5,7-dihydropyrrolo[3,4-d]pyrimidin-6-yl]-N,N-dimethyl-cyclohexanecarboxamide (20 mg, 11% yield). LCMS (ESI): m/z: [M+H] calculated for C₂₄H₃₁ClF₃N₆O: 511.2. found 511.1; H NMR (400 MHz, CHLOROFORM-d) δ ppm 6.96 (s, 1H) 6.82 (br d, J=9.66 Hz, 2H) 5.28-5.39 (m, 1H) 4.75 (br d, J=2.08 Hz, 1H) 3.92 (br s, 2H) 3.83 (br s, 2H) 3.75 (br s, 2H) 3.05 (s, 3H) 2.93 (s, 3H) 2.69 (br s, 1H) 2.61 (br s, 1H) 1.95 (br d, J=11.74 Hz, 4H) 1.58 (s, 3H) 1.45-1.55 (m, 4H).

### Example 22. Synthesis of N-[(1R)-1-[3-amino-5-(trifluoromethyl)phenyl]ethyl]-2-chloro-6-tetrahydropyran-4-yl-7,8-dihydro-5H-pyrido[4,3-d]pyrimidin-4-amine

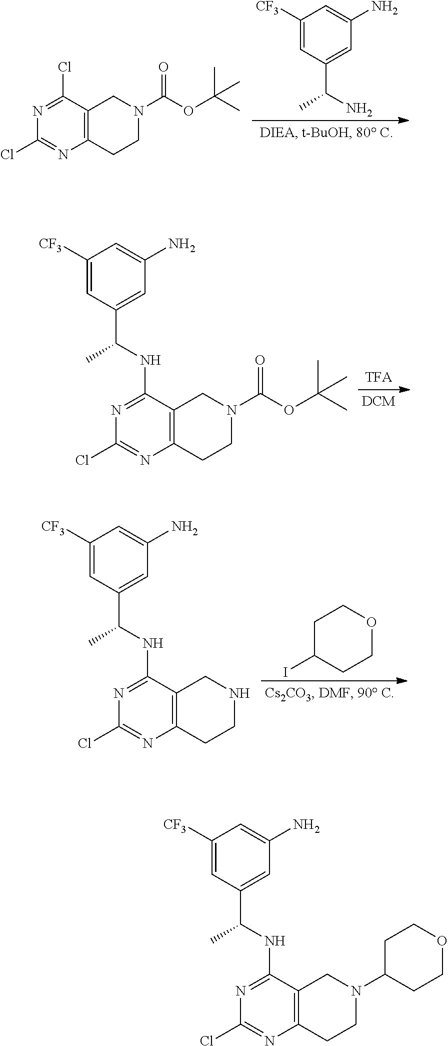 [SMILES: CC(C)(C)OC(=O)N1CCc2nc(Cl)nc(Cl)c2C1 ; C[C@@H](N)c1cc(N)cc(C(F)(F)F)c1 ; C[C@@H](Nc1nc(Cl)nc2c1CN(C(=O)OC(C)(C)C)CC2)c1cc(N)cc(C(F)(F)F)c1 ; C[C@@H](Nc1nc(Cl)nc2c1CNCC2)c1cc(N)cc(C(F)(F)F)c1 ; IC1CCOCC1 ; C[C@@H](Nc1nc(Cl)nc2c1CN(C1CCOCC1)CC2)c1cc(N)cc(C(F)(F)F)c1]

### Step 1.

To a mixture of tert-butyl 2,4-dichloro-7,8-dihydro-5H-pyrido[4,3-d]pyrimidine-6-carboxylate (150 mg, 493 μmol) in t-BuOH (0.5 mL) was added DIPEA (859 μL, 4.93 mmol) and 3-[(1R)-1-aminoethyl]-5-(trifluoromethyl)aniline (121 mg, 0.59 mmol). The mixture was stirred at 80° C. for 3 h, the solvent was removed under reduced pressure and the residue was purified by column chromatography to give tert-butyl 4-[[(1R)-1-[3-amino-5-(trifluoromethyl)phenyl]ethyl]amino]-2-chloro-7,8-dihydro-5H-pyrido[4,3-d]pyrimidine-6-carboxylate (0.15 g, 25% yield). ¹H NMR (400 MHz, METHANOL-d₄) δ ppm 6.94 (s, 1H), 6.91 (s, 1H), 6.82-6.79 (m, 1H), 5.45-5.30 (m, 1H), 4.39-4.31 (m, 2H).

### Step 2.

To a mixture of tert-butyl 4-[[(1R)-1-[3-amino-5-(trifluoromethyl)phenyl]ethyl]amino]-2-chloro-7,8-dihydro-5H-pyrido[4,3-d]pyrimidine-6-carboxylate (150 mg, 0.32 mmol) in DCM (0.9 mL) was added TFA (0.3 mL). The mixture was stirred at rt for 30 min, and the solvent was removed under reduced pressure to give N-[(1R)-1-[3-amino-5-(trifluoromethyl)phenyl]ethyl]-2-chloro-5,6,7,8-tetrahydropyrido[4,3-d]pyrimidin-4-amine TFA salt (0.15 g, 97% yield). ¹H NMR (400 MHz, METHANOL-d₄) δ ppm 7.46 (s, 1H), 7.39 (s, 1H), 7.26 (s, 1H), 5.48-5.33 (m, 1H), 4.18 (s, 2H), 3.56-3.53 (m, 2H), 2.96-2.93 (m, 2H), 1.61 (d, J=4.0 Hz, 3H).

### Step 3.

To a mixture of N-[(1R)-1-[3-amino-5-(trifluoromethyl)phenyl]ethyl]-2-chloro-5,6,7,8-tetrahydropyrido[4,3-d]pyrimidin-4-amine TFA salt (150 mg, 0.31 mmol) in DMF (1 mL) was added Cs₂CO₃ (805 mg, 2.47 mmol) and 4-iodotetrahydropyran (327 mg, 1.54 mmol). The mixture was stirred at 100° C. for 2 h, the solvent was removed under reduced pressure and the residue was purified by prep-HPLC (×2) to give N-[(1R)-1-[3-amino-5-(trifluoromethyl)phenyl]ethyl]-2-chloro-6-tetrahydropyran-4-yl-7,8-dihydro-5H-pyrido[4,3-d]pyrimidin-4-amine (5 mg, 3.5% yield). LCMS (ESI): m/z: [M+H] calculated for C₂₁H₂₆ClF₃N₅O: 456.2. found 456.2; H NMR (400 MHz, METHANOL-d₄) δ ppm 6.91 (s, 1H) 6.89 (s, 1H) 6.80 (s, 1H) 5.42-5.33 (m, 1H) 4.08-4.00 (m, 2H) 3.54-3.40 (m, 4H) 2.92-2.87 (m, 2H) 2.75-2.71 (m, 3H) 1.93 (d, J=12.0 Hz, 2H) 1.74-1.64 (m, 2H) 1.55 (d, J=4.0 Hz, 3H).

### Example 177. Synthesis of (R)-6-(azetidin-3-yl)-2-chloro-N-(1-(3-(trifluoromethyl)phenyl)ethyl)-5,6,7,8-tetrahydropyrido[4,3-d]pyrimidin-4-amine

(R)-6-(azetidin-3-yl)-2-chloro-N-(1-(3-(trifluoromethyl)phenyl)ethyl)-5,6,7,8-tetrahydropyrido[4,3-d]pyrimidin-4-amine was synthesized in the manner similar to Example 22.

| Example | | Mass |
|---|---|---|
| # | Structure | found |
| Example 177. | 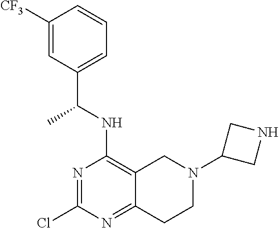 [SMILES: C[C@@H](Nc1nc(Cl)nc2c1CN(C1CNC1)CC2)c1cccc(C(F)(F)F)c1] | 412.4 |

### Example 23. Synthesis of [2-chloro-4-[1-[5-[2-[(4-methylpiperazin-1-yl)methyl]phenyl]-2-thienyl]ethylamino]-5,7-dihydropyrrolo[3,4-d]pyrimidin-6-yl]-morpholino-methanone

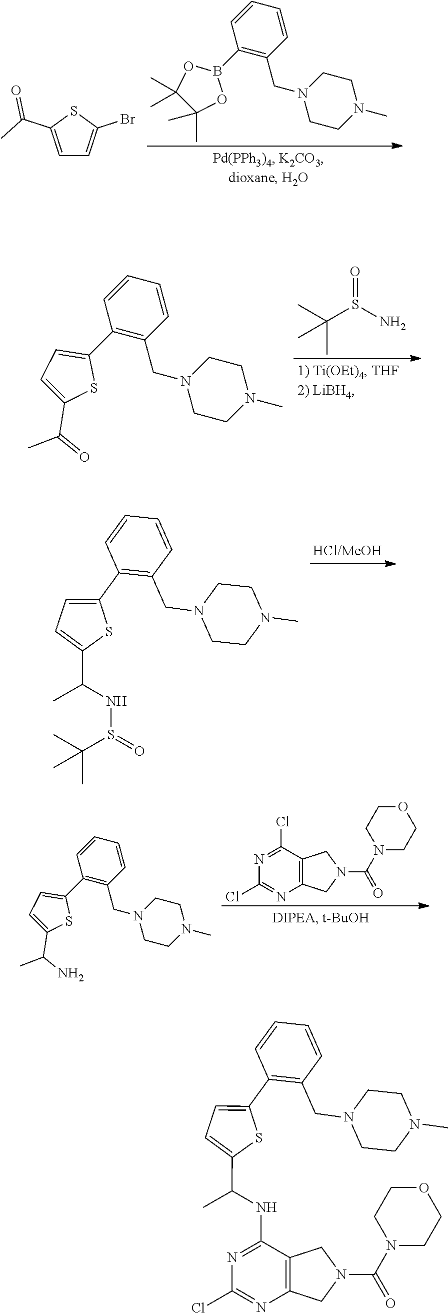

### Step 1.

A mixture of 1-methyl-4-[[2-(4,4,5,5-tetramethyl-1,3,2-dioxaborolan-2-yl)phenyl]methyl]piperazine (1.7 g, 5.4 mmol), 1-(5-bromo-2-thienyl)ethanone (1.21 g, 5.91 mmol), K₂CO₃ (2.23 g, 16.13 mmol), Pd(PPh₃)₄(621 mg, 538 μmol) in 1,4-dioxane (5 mL) and H₂O (1 mL) was degassed and purged with N₂ (×3). The mixture was heated to 110° C. and stirred for 4 h under an atmosphere of N₂, then diluted with H₂O (10 mL) and extracted with EtOAc (5 mL×3). The combined organic layers were washed with brine (10 mL), dried over Na₂SO₄ and filtered. The solvent was concentrated under reduced pressure and the crude residue was purified by column chromatography to give 1-[5-[2-[(4-methylpiperazin-1-yl)methyl]phenyl]-2-thienyl]ethenone (1 g, 59% yield). LCMS (ESI): m/z: [M+H] calculated for C₁₈H₂₃N₂OS: 315.15. found 315.1.

### Step 2.

To a mixture of 1-[5-[2-[(4-methylpiperazin-1-yl)methyl]phenyl]-2-thienyl]ethanone (800 mg, 2.54 mmol) in THF (5 mL) was added 2-methylpropane-2-sulfinamide (617 mg, 5.09 mmol) and Ti(OEt)₄ (2.1 mL, 10.2 mmol). The mixture was heated to 80° C. and stirred for 16 h. The mixture was cooled to 0° C., then LiBH₄ (222 mg, 10.2 mmol) and MeOH (103 μL, 2.54 mmol) were added, and the mixture warmed to rt and stirred for 1 h. H₂O (25 mL) was added, the mixture was filtered, concentrated under reduced pressure and the residue was purified by column chromatography to give 2-methyl-N-[1-[5-[2-[(4-methylpiperazin-1-yl)methyl]phenyl]-2-thienyl] ethyl]propane-2-sulfinamide (620 mg, 58% yield). ¹H NMR (400 MHz, METHANOL-d₄) δ ppm 7.49-7.30 (m, 5H), 7.09-7.06 (m, 2H), 4.78 (q, J=6.8 Hz, 1H), 3.33 (s, 2H), 3.04-2.97 (m, 2H), 2.77 (dd, J=8.8, 4.4 Hz, 5H), 2.55-2.50 (m, 2H), 1.68 (d, J=6.4 Hz, 3H), 1.23 (s, 9H).

### Step 3.

A mixture of 2-methyl-N-[1-[5-[2-[(4-methylpiperazin-1-yl)methyl]phenyl]-2-thienyl] ethyl]propane-2-sulfinamide (620 mg, 1.48 mmol) in 4M HCl in MeOH (1 mL, 4 mmol) was stirred at rt for 1 h. The mixture was concentrated under reduced pressure to give 1-[5-[2-[(4-methylpiperazin-1-yl)methyl]phenyl]-2-thienyl]ethanamine (300 mg, 64% yield).

### Step 4.

To a mixture of 1-[5-[2-[(4-methylpiperazin-1-yl)methyl]phenyl]-2-thienyl]ethanamine (100 mg, 0.32 mmol) in t-BuOH (1 mL) was added DIPEA (221 μL, 1.27 mmol) and (2,4-dichloro-5,7-dihydropyrrolo[3,4-d]pyrimidin-6-yl)-morpholino-methanone (96 mg, 0.32 mmol). The mixture was heated to 80° C. and stirred for 4 h, the solvent was concentrated under reduced pressure and the residue was purified by prep-HPLC to give [2-chloro-4-[1-[5-[2-[(4-methylpiperazin-1-yl)methyl]phenyl]-2-thienyl]ethylamino]-5,7-dihydropyrrolo[3,4-d]pyrimidin-6-yl]-morpholino-methanone (50 mg, 24% yield). LCMS (ESI): m/z: [M+H] calculated for C₂₉H₃₇ClN₇O₂S: 582.23. found 582.3; ¹H NMR (400 MHz, METHANOL-d₄) δ ppm 7.43-7.37 (m, 2H), 7.34-7.28 (m, 2H), 7.10-7.04 (m, 2H), 5.75 (d, J=6.8 Hz, 1H), 4.61 (s, 4H), 3.75-3.70 (m, 4H), 3.52 (s, 2H), 3.39-3.35 (m, 4H), 2.41 (s, 8H), 2.25 (s, 3H), 1.72 (d, J=6.8 Hz, 3H).

### Example 24. Synthesis of N-[(1R)-1-[3-amino-5-(trifluoromethyl)phenyl]ethyl]-2-chloro-6-morpholinosulfonyl-5,7-dihydropyrrolo[3,4-d]pyrimidin-4-amine

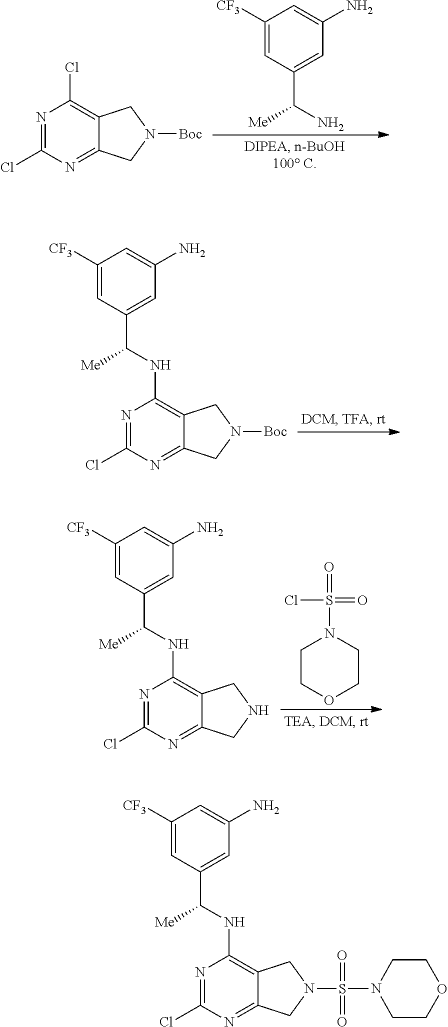 [SMILES: CC(C)(C)OC(=O)N1Cc2nc(Cl)nc(Cl)c2C1 ; C[C@@H](N)c1cc(N)cc(C(F)(F)F)c1 ; C[C@@H](Nc1nc(Cl)nc2c1CN(C(=O)OC(C)(C)C)C2)c1cc(N)cc(C(F)(F)F)c1 ; C[C@@H](Nc1nc(Cl)nc2c1CNC2)c1cc(N)cc(C(F)(F)F)c1 ; O=S(=O)(Cl)N1CCOCC1 ; C[C@@H](Nc1nc(Cl)nc2c1CN(S(=O)(=O)N1CCOCC1)C2)c1cc(N)cc(C(F)(F)F)c1]

### Step 1.

To a mixture of tert-butyl 2,4-dichloro-5,7-dihydropyrrolo[3,4-d]pyrimidine-6-carboxylate (200 mg, 0.69 mmol) and 3-[(1R)-1-aminoethyl]-5-(trifluoromethyl)aniline (169 mg, 0.83 mmol) in n-BuOH (4 mL) was added DIPEA (1.2 mL, 6.9 mmol). The mixture was heated to 100° C. and stirred for 2 h, the solvent was concentrated under reduced pressure and the residue was purified by column chromatography to give tert-butyl 4-[[(1R)-1-[3-amino-5-(trifluoromethyl)phenyl]ethyl]amino]-2-chloro-5,7-dihydropyrrolo[3,4-d]pyrimidine-6-carboxylate (300 mg, 95% yield). LCMS (ESI): m/z: [M+H] calculated for C₂₀H₂₄ClF₃N₅O₂: 458.15. found 458.2.

### Step 2.

A mixture of tert-butyl 4-[[(1R)-1-[3-amino-5-(trifluoromethyl)phenyl]ethyl]amino]-2-chloro-5,7-dihydropyrrolo[3,4-d]pyrimidine-6-carboxylate (150 mg, 0.33 mmol) in TFA (1 mL) and DCM (3 mL) was stirred at rt for 30 min. The mixture was concentrated under reduced pressure to give N-[(1R)-1-[3-amino-5-(trifluoromethyl)phenyl]ethyl]-2-chloro-6,7-dihydro-5H-pyrrolo[3,4-d]pyrimidin-4-amine TFA salt (154 mg, 100% yield).

### Step 3.

To a mixture of N-[(1R)-1-[3-amino-5-(trifluoromethyl)phenyl]ethyl]-2-chloro-6,7-dihydro-5H-pyrrolo[3,4-d]pyrimidin-4-amine (154 mg, 0.33 mmol) and morpholine-4-sulfonyl chloride (61 mg, 0.33 mmol) in DCM (3 mL) at 0° C. was added TEA (454 μL, 3.3 mmol) dropwise. The mixture was warmed to rt, stirred for 1 h and filtered. The solvent was concentrated under reduced pressure and the residue was purified by prep-HPLC to give N-[(1R)-1-[3-amino-5-(trifluoromethyl)phenyl]ethyl]-2-chloro-6-morpholinosulfonyl-5,7-dihydropyrrolo[3,4-d]pyrimdin-4-amine (46 mg, 275 yield). LCMS (ESI): m/z: [M+H] calculated for C₁₉H₂₃ClF₃N₆O₃S: 507.11. found 507.1; ¹H NMR (400 MHz, DMSO-d₆) δ ppm 8.12 (d, J=8.2 Hz, 1H), 6.82 (s, 1H), 6.76 (s, 1H), 6.71 (s, 1H), 5.57 (s, 2H), 5.18 (t, J=7.2 Hz, 1H), 4.52-4.40 (m, 4H), 3.67-3.59 (m, 4H), 3.20-3.13 (m, 4H), 1.43 (d, J=7.0 Hz, 3H).

The examples in the following Table 1 were synthesized in the manner similar to Example 24.

TABLE 1
Examples 25-28 and 178

| Example # | Structure | Mass found |
|---|---|---|
| Example 25. | 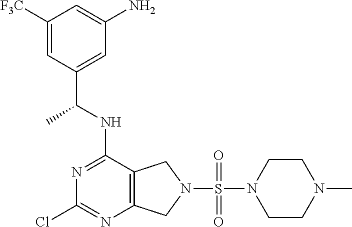 [SMILES: C[C@@H](Nc1nc(Cl)nc2c1CN(S(=O)(=O)N1CCN(C)CC1)C2)c1cc(N)cc(C(F)(F)F)c1] | 520.5 |
| Example 26. | 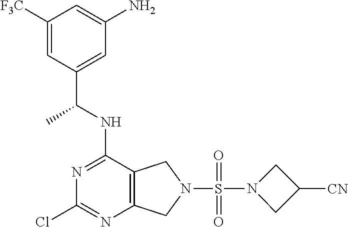 [SMILES: C[C@@H](Nc1nc(Cl)nc2c1CN(S(=O)(=O)N1CC(C#N)C1)C2)c1cc(N)cc(C(F)(F)F)c1] | 502.5 |
| Example 27. | 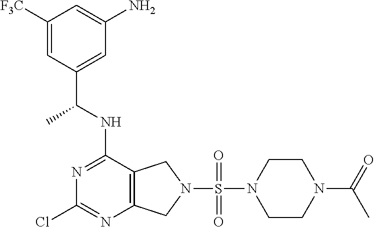 [SMILES: CC(=O)N1CCN(S(=O)(=O)N2Cc3nc(Cl)nc(N[C@H](C)c4cc(N)cc(C(F)(F)F)c4)c3C2)CC1] | 574.4 |
| Example 28. | 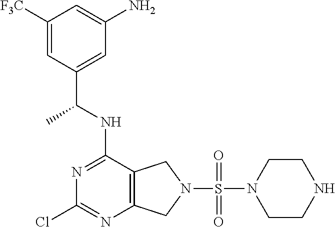 [SMILES: C[C@@H](Nc1nc(Cl)nc2c1CN(S(=O)(=O)N1CCNCC1)C2)c1cc(N)cc(C(F)(F)F)c1] | 506.2 |
| Example 178. | 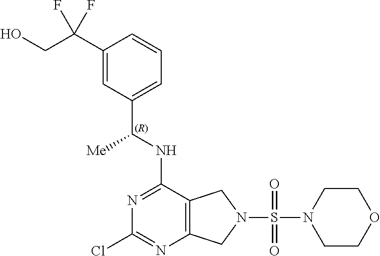 | 504.1 |

### Example 29. Synthesis of N-[(1R)-1-[3-amino-5-(trifluoromethyl)phenyl]ethyl]-2-chloro-6-morpholinosulfonyl-7,8-dihydro-5H-pyrido[4,3-d]pyrimidin-4-amine

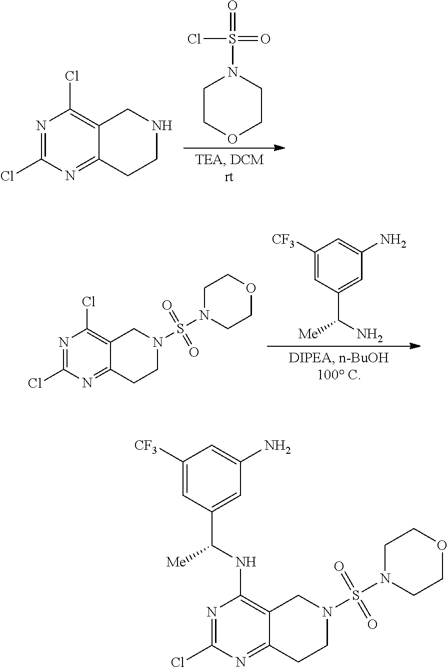 [SMILES: Clc1nc(Cl)c2c(n1)CCNC2 ; O=S(=O)(Cl)N1CCOCC1 ; O=S(=O)(N1CCOCC1)N1CCc2nc(Cl)nc(Cl)c2C1 ; C[C@@H](N)c1cc(N)cc(C(F)(F)F)c1 ; C[C@@H](Nc1nc(Cl)nc2c1CN(S(=O)(=O)N1CCOCC1)CC2)c1cc(N)cc(C(F)(F)F)c1]

### Step 1.

To a mixture of 2,4-dichloro-5,6,7,8-tetrahydropyrido[4,3-d]pyrimidine TFA salt (209 mg, 0.66 mmol) and morpholine-4-sulfonyl chloride (610 mg, 3.3 mmol) in DCM (6 mL) at 0° C. was added TEA (1.83 mL, 13.1 mmol) dropwise. The mixture was stirred at 0° C. for 30 min, then the solvent was concentrated under reduced pressure and the residue was purified by column chromatography to give 4-[(2,4-dichloro-7,8-dihydro-5H-pyrido[4,3-d]pyrimidin-6-yl)sulfonyl]morpholine (160 mg, 69% yield). LCMS (ESI): m/z: [M+H] calculated for C₁₁H₁₅Cl₂N₄O₃S: 353.02. found 353.0; ¹H NMR (400 MHz, DMSO-d₆) δ ppm 4.40 (s, 2H), 3.63 (dd, J=5.4, 2.5 Hz, 6H), 3.20-3.14 (m, 4H), 3.01 (s, 2H).

### Step 2.

To a mixture of 4-[(2,4-dichloro-7,8-dihydro-5H-pyrido[4,3-d]pyrimidin-6-yl) sulfonyl]morpholine (160 mg, 0.45 mmol) and 3-[(1R)-1-aminoethyl]-5-(trifluoromethyl)aniline (111 mg, 0.54 mmol) in n-BuOH (4 mL) was added DIPEA (789 QL, 4.53 mmol). The mixture was heated to 100° C. and stirred for 10 h. The mixture was filtered, the solvent was concentrated under reduced pressure and the residue was purified by prep-HPLC to give N-[(1R)-1-[3-amino-5-(trifluoromethyl)phenyl]ethyl]-2-chloro-6-morpholinosulfonyl-7,8-dihydro-5H-pyrido[4,3-d]pyrimidin-4-amine (96 mg, 41% yield). LCMS (ESI): m/z: [M+H] calculated for C₂₀H₂₅ClF₃N₆O₃S: 521.13. found 521.1; ¹H NMR (400 MHz, DMSO-d₆) δ ppm 7.68 (d, J=7.8 Hz, 1H), 6.83 (s, 1H), 6.77 (s, 1H), 6.71 (s, 1H), 5.57 (s, 2H), 5.25 (t, J=7.3 Hz, 1H), 4.18 (s, 2H), 3.64-3.60 (m, 4H), 3.52 (t, J=5.7 Hz, 2H), 3.17-3.13 (m, 4H), 2.70 (t, J=5.0 Hz, 2H), 1.47 (d, J=7.0 Hz, 3H).

The examples in the following Table 2 were synthesized in the manner similar to Example 29.

TABLE 2
Examples 179-185

| Example # | Structure | Mass found |
|---|---|---|
| Example 179. | 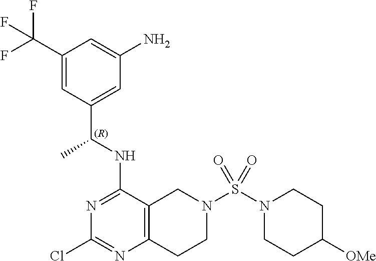 | 549.6 |
| Example 180. | 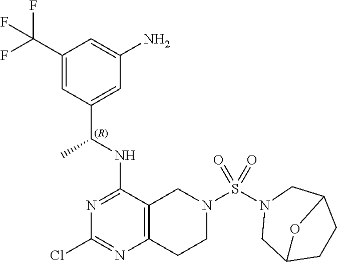 | 547.5 |
| Example 181. | 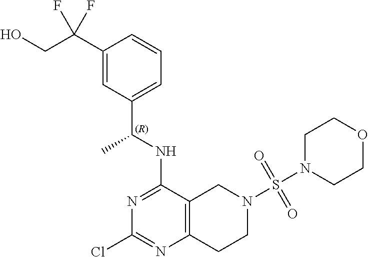 | 518.2 |
| Example 182. | 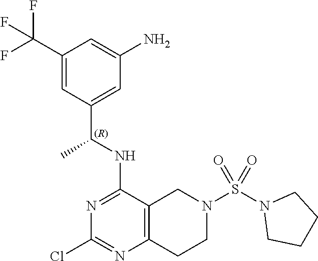 | 505.5 |
| Example 183. | 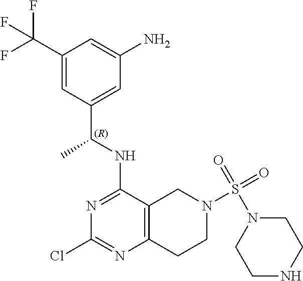 | 520.5 |
| Example 184. | 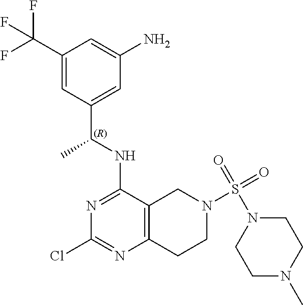 | 534.5 |
| Example 185. | 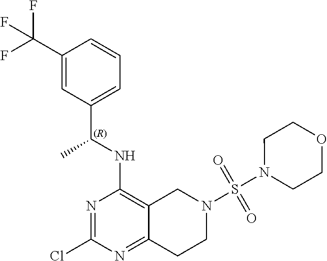 | 506.4 |

### Example 30. Synthesis of 4-[[(1R)-1-[3-amino-5-(trifluoromethyl)phenyl]ethyl]amino]-2-chloro-6-tetrahydropyran-4-yl-5H-pyrrolo[3,4-d]pyrimidin-7-one

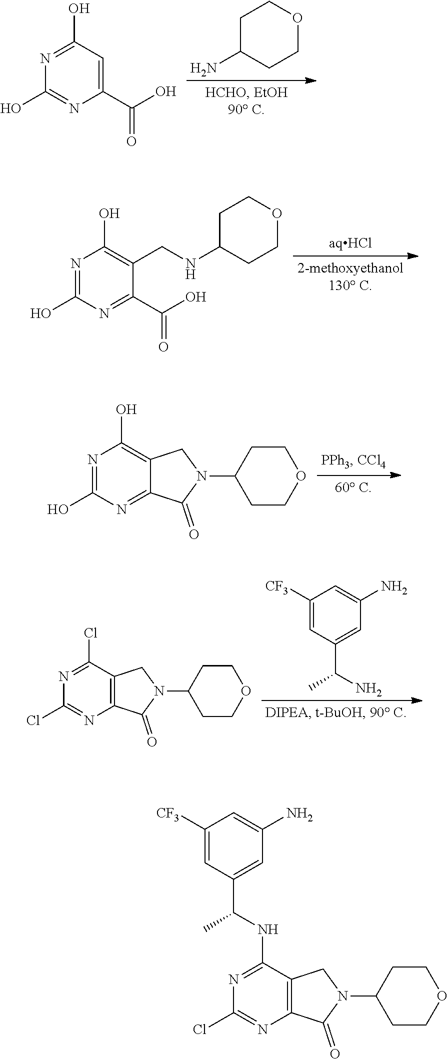

### Step 1.

To a mixture of 2,6-dihydroxypyrimidine-4-carboxylic acid (200 mg, 1.28 mmol) and tetrahydropyran-4-amine (194 mg, 1.92 mmol) in EtOH (4 mL) was added 37% aqueous HCHO (0.95 mL, 12.8 mmol). The mixture was heated to 90° C. and stirred for 10 h in a crimped vial. After cooling, H₂O (1 mL) was added, the mixture was filtered, and the filter cake was dried to give 2,6-dihydroxy-5-[(tetrahydropyran-4-ylamino)methyl]pyrimidine-4-carboxylic acid (120 mg, 35% yield). LCMS (ESI): m/z: [M−H] calculated for C₁₁H₁₄N₃O₅: 268.1. found 268.0; ¹H NMR (400 MHz, D₂O) δ ppm 3.91 (dd, J=12.0, 2.4 Hz, 2H), 3.49 (s, 2H), 3.40 (dt, J=12.0, 1.6 Hz, 2H), 2.65 (tt, J=10.8, 4.0 Hz, 1H), 1.78 (dd, J=12.8, 2.0 Hz, 2H), 1.38-1.23 (m, 2H).

### Step 2.

To a mixture of 2,6-dihydroxy-5-[(tetrahydropyran-4-ylamino)methyl]pyrimidine-4-carboxylic acid (120 mg, 0.45 mmol) in 2-methoxyethanol (0.7 mL) was added 12M HCl (0.37 mL, 4.5 mmol). The mixture was heated to 130° C. and stirred for 18 h. The mixture was cooled to rt, filtered and the filter cake dried to give 2,4-dihydroxy-6-tetrahy dropyran-4-yl-5H-pyrrolo[3,4-d]pyrimidin-7-one (100 mg, 89% yield). ¹H NMR (400 MHz, DMSO-d₆) δ ppm 11.83 (br s, 1H), 11.27 (br s, 1H), 4.18 (s, 2H), 4.16-4.07 (m, 1H), 3.91 (dd, J=11.2, 3.6 Hz, 2H), 3.41 (t, J=11.2 Hz, 2H), 1.78 (dq, J=12.0, 4.0 Hz, 2H), 1.68-1.60 (m, 2H).

### Step 3.

To a mixture of 2,4-dihydroxy-6-tetrahydropyran-4-yl-5H-pyrrolo[3,4-d]pyrimidin-7-one (100 mg, 0.4 mmol) in DCE (3 mL) was added PPh₃ (418 mg, 1.59 mmol) and CCl₄ (0.19 mL, 2.0 mmol). The mixture was heated to 60° C. and stirred for 12 h, then filtered. The solvent was concentrated under reduced pressure to give 2,4-dichloro-6-tetrahydropyran-4-yl-5H-pyrrolo[3,4-d]pyrimidin-7-one (75 mg, 65% yield). LCMS (ESI): m/z: [M+H] calculated for C₁₁H₁₂Cl₂N₃O₂: 288.02. found 288.0.

### Step 4.

To a mixture of 2,4-dichloro-6-tetrahydropyran-4-yl-5H-pyrrolo[3,4-d]pyrimidin-7-one (75 mg, 0.26 mmol) in t-BuOH (0.5 mL) was added 3-[(1R)-1-aminoethyl]-5-(trifluoromethyl)aniline (64 mg, 0.31 mmol) and DIPEA (91 μL, 0.52 mmol). The mixture was heated to 90° C. in a crimped vial and stirred for 30 min, then filtered. The solvent was concentrated under reduced pressure and the residue was purified by prep-HPLC to give 4-[[(1R)-1-[3-amino-5-(trifluoromethyl)phenyl]ethyl]amino]-2-chloro-6-tetrahydropyran-4-yl-5H-pyrrolo[3,4-d]pyrimidin-7-one (55 mg, 45% yield). LCMS (ESI): m/z: [M+H] calculated for C₂₀H22ClF₃N5O₂: 456.13. found 456.1; ¹H NMR (400 MHz, DMSO-d₆) δ ppm 8.60 (br d, J=7.8 Hz, 1H), 6.85 (s, 1H), 6.80 (s, 1H), 6.72 (s, 1H), 5.56 (br s, 2H), 5.24 (t, J=7.2 Hz, 1H), 4.32 (s, 2H), 4.28-4.16 (m, 1H), 3.96 (m, 2H), 3.48-3.32 (m, 2H), 1.73 (m, 4H), 1.48 (d, J=6.8 Hz, 3H).

### Example 31. Synthesis of 2-chloro-4-[1-[5-[2-(methylaminomethyl)phenyl]thiazol-2-yl]ethylamino]-5,7-dihydropyrrolo[3,4-d]pyrimidin-6-yl]-morpholino-methanone

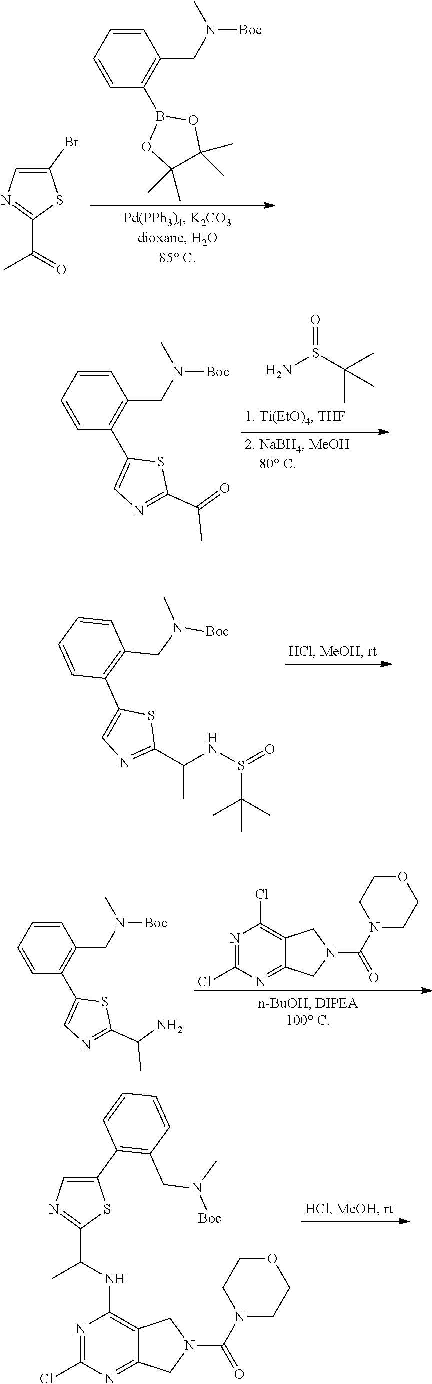

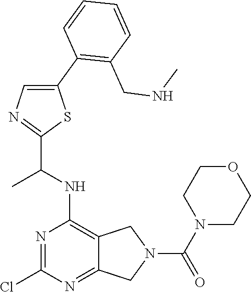 [SMILES: CNCc1ccccc1-c1cnc(C(C)Nc2nc(Cl)nc3c2CN(C(=O)N2CCOCC2)C3)s1]

### Step 1.

To a mixture of 1-(5-bromothiazol-2-yl)ethanone (0.8 g, 3.9 mmol) in 1,4-dioxane (8 mL) and H₂O (1.6 mL) was added K₂CO₃ (2.15 g, 15.5 mmol), tert-butyl N-methyl-N-[[2-(4,4,5,5-tetramethyl-1,3,2-dioxaborolan-2-yl)phenyl]methyl]carbamate (2.02 g, 5.8 mmol) and Pd(PPh₃)₄(449 mg, 0.4 mmol) under an atmosphere of N₂. The mixture was heated to 85° C. and stirred for 16 h, then cooled and filtered. The solvent was concentrated under reduced pressure and the residue was purified by column chromatography to give tert-butyl N-[[2-(2-acetylthiazol-5-yl)phenyl]methyl]-N-methyl-carbamate (0.8 g, 60% yield). LCMS (ESI): m/z: [M+H-^{t}Bu] calculated for C₁₄H₁₅N₂O₃S: 291.0. found 291.0.

### Step 2.

To a mixture of tert-butyl N-[[2-(2-acetylthiazol-5-yl)phenyl]methyl]-N-methyl-carbamate (1.2 g, 3.46 mmol) in THF (20 mL) was added 2-methylpropane-2-sulfinamide (630 mg, 5.2 mmol) and Ti(OEt)₄ (2.51 mL, 12.1 mmol). The mixture was heated to 80° C. and stirred for 2 h. The mixture was cooled to 0° C., MeOH (140 μL, 3.5 mmol) and NaBH₄ (262 mg, 6.93 mmol) were added slowly, then warmed to rt and stirred for 30 min. The mixture was filtered, the solvent was concentrated under reduced pressure and the residue was purified by column chromatography to give tert-butyl N-[[2-[2-[1-(tert-butylsulfinylamino)ethyl]thiazol-5-yl]phenyl]methyl]-N-methyl-carbamate (1 g, 64% yield). LCMS (ESI): m/z: [M+H] calculated for C₂₂H₃₄N₃O₃S₂: 452.2. found 452.2.

### Step 3.

To a mixture of tert-butyl N-[[2-[2-[1-(tert-butylsulfinylamino)ethyl]thiazol-5-yl] phenyl]methyl]-N-methyl-carbamate (0.6 g, 1.33 mmol) in MeOH (20 mL) was added 4M HCl in MeOH (664 μL, 2.66 mmol). The mixture was stirred at rt for 1 h, and the solvent was concentrated under reduced pressure to give tert-butyl N-[[2-[2-(1-aminoethyl)thiazol-5-yl]phenyl]methyl]-N-methyl-carbamate (462 mg), which was used directly in the next step without further purification. LCMS (ESI): m/z: [M+H] calculated for C₁₈H₂₆N₃O₂S:348.2. found 348.2; ¹H NMR (400 MHz, CDCl₃) δ ppm 7.70-7.62 (m, 1H), 7.50-7.29 (m, 4H), 4.98-4.89 (m, 1H), 4.62-4.41 (m, 3H), 2.86-2.67 (m, 4H), 2.01-1.90 (m, 3H), 1.52-1.49 (m, 3H).

### Step 4.

To a mixture of tert-butyl N-[[2-[2-(1-aminoethyl)thiazol-5-yl]phenyl]methyl]-N-methyl-carbamate (462 mg, 1.33 mmol) in n-BuOH (5 mL) was added (2,4-dichloro-5,7-dihydropyrrolo[3,4-d]pyrimidin-6-yl)-morpholino-methanone (403 mg, 1.33 mmol) and DIPEA (695 μL, 4.0 mmol). The mixture was heated to 100° C. and stirred for 3 h, the solvent was concentrated under reduced pressure and the residue was purified by column chromatography to give tert-butyl N-[[2-[2-[1-[[2-chloro-6-(morpholine-4-carbonyl)-5,7-dihydropyrrolo[3,4-d]pyrimidin-4-yl]amino]ethyl]thiazol-5-yl]phenyl]methyl]-N-methyl-carbamate (0.3 g, 37% yield). LCMS (ESI): m/z: [M+H] calculated for C₂₉H₃₇ClN₇O₄S: 614.2. found 614.2.

### Step 5.

A mixture of tert-butyl N-[[2-[2-[1-[[2-chloro-6-(morpholine-4-carbonyl)-5,7-dihydropyrrolo[3,4-d]pyrimidin-4-yl]amino]ethyl]thiazol-5-yl]phenyl]methyl]-N-methyl-carbamate (200 mg, 0.33 mmol) in 4M HCl in MeOH (5 mL) was stirred at rt for 1 h. The solvent was concentrated under reduced pressure and the residue was purified by prep-HPLC to give 2-chloro-4-[1-[5-[2-(methylaminomethyl)phenyl]thiazol-2-yl]ethylamino]-5,7-dihydropyrrolo[3,4-d]pyrimidin-6-yl]-morpholino-methanone (40 mg, 23% yield). LCMS (ESI): m/z: [M+H] calculated for C₂₄H₂₉ClN₇O₂S: 514.2. found 514.2; ¹H NMR (400 MHz, METHANOL-d₄) δ ppm 8.54-8.42 (m, 1H), 7.71 (s, 1H), 7.63-7.44 (m, 4H), 5.78-5.66 (m, 1H), 4.69-4.58 (m, 4H), 4.25-4.17 (m, 2H), 3.77-3.68 (m, 4H), 3.39-3.33 (m, 4H), 2.61 (s, 3H), 1.77 (d, J=7.1 Hz, 2H).

### Examples 32. and 33. Synthesis of [2-chloro-4-[1-[3-[2-(methylaminomethyl)phenyl]phenyl]ethylamino]-5,7-dihydropyrrolo[3,4-d]pyrimidin-6-yl]-morpholino-methanone and [2-methoxy-4-[1-[3-[2-(methylaminomethyl)phenyl]phenyl]ethylamino]-5,7-dihydropyrrolo[3,4-d]pyrimidin-6-yl]-morpholino-methanone

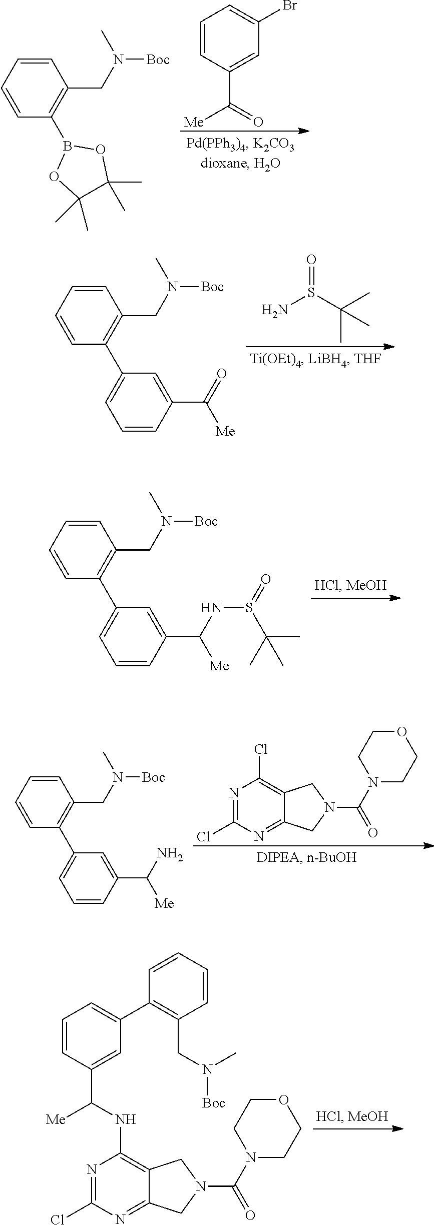

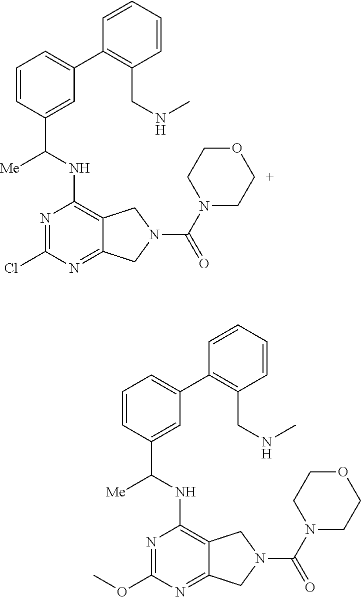

### Step 1.

To a mixture of tert-butyl N-methyl-N-[[2-(4,4,5,5-tetramethyl-1,3,2-dioxaborolan-2-yl) phenyl]methyl]carbamate (2.62 g, 7.54 mmol) in 1,4-dioxane (16 mL) and H₂O (3.5 mL) was added 1-(3-bromophenyl)ethanone (1 g, 5.0 mmol), Pd(PPh₃)₄(581 mg, 0.5 mmol) and K₂CO₃ (2.78 g, 20.1 mmol). The mixture was heated to 85° C. and stirred for 16 h. The mixture was cooled, filtered, the solvent was concentrated under reduced pressure and the residue was purified by column chromatography to give tert-butyl N-[[2-(3-acetylphenyl)phenyl]methyl]-N-methyl-carbamate (1.5 g, 4.42 mmol, 88% yield). H NMR (400 MHz, METHANOL-d₄) δ ppm 8.00 (d, J=6.2 Hz, 1H), 7.89 (s, 1H), 7.61-7.50 (m, 2H), 7.43-7.33 (m, 2H), 7.29 (d, J=7.6 Hz, 1H), 7.23 (d, J=7.2 Hz, 1H), 4.40 (s, 2H), 2.63 (s, 5H), 1.47-1.29 (m, 9H).

### Step 2.

To a mixture of tert-butyl N-[[2-(3-acetylphenyl)phenyl]methyl]-N-methyl-carbamate (1 g, 2.95 mmol) in THF (10 mL) was added 2-methylpropane-2-sulfinamide (714 mg, 5.89 mmol) and Ti(OEt)₄ (2.44 mL, 11.78 mmol) at rt over 5 min. After addition, the mixture was heated to 80° C. and stirred for 12 h. The mixture was cooled to 0° C., LiBH₄ (257 mg, 11.78 mmol) was added, and the mixture was stirred at 0° C. for 2 h. H₂O (5 mL) was added at 0° C. and the mixture was filtered. The solvent was concentrated under reduced pressure and the residue was purified by column chromatography to give tert-butyl N-[[2-[3-[1-(tert-butylsulfinylamino)ethyl]phenyl]phenyl]methyl]-N-methyl-carbamate (500 mg, 38% yield). ¹H NMR (400 MHz, METHANOL-d₄) δ ppm 7.44-7.38 (m, 2H), 7.37-7.30 (m, 3H), 7.24 (m, 2H), 7.20-7.16 (m, 1H), 4.56-4.49 (m, 1H), 4.41 (d, J=3.5 Hz, 1H), 2.78-2.50 (m, 3H), 1.54 (d, J=6.8 Hz, 3H), 1.45-1.34 (m, 9H), 1.24 (s, 9H).

### Step 3.

To a mixture of tert-butyl N-[[2-[3-[1-(tert-butylsulfinylamino)ethyl]phenyl]phenyl]methyl]-N-methyl-carbamate (500 mg, 1.12 mmol) in MeOH (20 mL) was added 4M HCl in MeOH (562 μL, 2.24 mmol). The mixture was stirred at rt for 1 h, then NaOH in MeOH (5 mL) was added dropwise until pH ˜8. The solvent was concentrated under reduced pressure and the residue was suspended in DCM/MeOH (20 mL), stirred for 30 min, filtered and the solvent concentrated under reduced pressure to give tert-butyl N-[[2-[3-(1-aminoethyl)phenyl]phenyl]methyl]-N-methyl-carbamate (400 mg). ¹H NMR (400 MHz, METHANOL-d₄) δ ppm 7.55-7.49 (m, 1H), 7.45 (d, J=6.5 Hz, 1H), 7.42-7.30 (m, 4H), 7.28 (d, J=7.5 Hz, 1H), 7.25-7.21 (m, 1H), 4.49-4.38 (m, 3H), 2.66 (s, 3H), 1.63 (d, J=6.8 Hz, 3H), 1.49-1.33 (m, 9H).

### Step 4.

To a mixture of (2,4-dichloro-5,7-dihydropyrrolo[3,4-d]pyrimidin-6-yl)-morpholino-methanone (178 mg, 0.58 mmol) in n-BuOH (3 mL) was added tert-butyl N-[[2-[3-(1-aminoethyl)phenyl]phenyl]methyl]-N-methyl-carbamate (200 mg, 0.58 mmol) and DIPEA (307 μL, 1.76 mmol). The mixture was heated to 100° C. and stirred for 3 h, then the solvent was concentrated under reduced pressure and the residue was purified by column chromatography to give tert-butyl N-[[2-[4-[1-[[2-chloro-6-(morpholine-4-carbonyl)-5,7-dihydropyrrolo[3,4-d]pyrimidin-4-yl]amino]ethyl]thiazol-2-yl]phenyl]methyl]-N-methyl-carbamate (250 mg, 69% yield). LCMS (ESI): m/z: [M+H] calculated for C₃₂H₄₀ClN₆O₄: 607.3. found 607.3; ¹H NMR (400 MHz, METHANOL-d₄) δ ppm 7.44-7.37 (m, 2H), 7.36-7.27 (m, 3H), 7.23 (t, J=6.3 Hz, 2H), 7.16 (s, 1H), 5.37 (d, J=6.5 Hz, 1H), 4.83 (s, 1H), 4.67-4.51 (m, 4H), 4.42-4.26 (m, 2H), 3.75-3.68 (m, 4H), 3.39-3.33 (m, 4H), 2.59 (d, J=17.1 Hz, 3H), 1.61 (d, J=7.1 Hz, 3H), 1.47-1.29 (m, 9H).

### Step 5.

A mixture of tert-butyl N-[[2-[3-[1-[[2-chloro-6-(morpholine-4-carbonyl)-5,7-dihydropyrrolo[3,4-d]pyrimidin-4-yl]amino]ethyl]phenyl]phenyl]methyl]-N-methyl-carbamate (100 mg, 0.16 mmol) in 4M HCl in MeOH (2 mL) was stirred at rt for 1 h. The solvent was concentrated under reduced pressure, then diluted with MeOH (10 mL) and the pH adjusted to ˜8 by dropwise addition of NaOH in MeOH. The solvent was concentrated under reduced pressure and the residue was purified by prep-HPLC to give [2-chloro-4-[1-[3-[2-(methylaminomethyl)phenyl]phenyl]ethylamino]-5,7-dihydropyrrolo[3,4-d]pyrimidin-6-yl]-morpholino-methanone (5.6 mg, 6% yield) and [2-methoxy-4-[1-[3-[2-(methylaminomethyl)phenyl]phenyl]ethylamino]-5,7-dihydropyrrolo[3,4-d]pyrimidin-6-yl]-morpholino-methanone (3.6 mg, 4% yield). LCMS (ESI): m/z: [M+H] calculated for C₂₇H₃₂ClN₆O₂: 507.2. found 507.2; ¹H NMR (400 MHz, METHANOL-d₄) δ ppm 7.49-7.39 (m, 4H), 7.38-7.31 (m, 3H), 7.27-7.18 (m, 2H), 5.41 (d, J=6.6 Hz, 1H), 4.65-4.54 (m, 4H), 3.75-3.67 (m, 7H), 3.38-3.33 (m, 5H), 2.20 (s, 3H), 1.61 (d, J=7.1 Hz, 3H); LCMS (ESI): m/z: [M+H] calculated for C₂₈H₃₅N₆O₃: 503.2. found 503.3; ¹H NMR (400 MHz, METHANOL-d₄) δ ppm 7.47-7.37 (m, 3H), 7.37-7.29 (m, 3H), 7.24-7.16 (m, 2H), 5.46-5.31 (m, 1H), 4.59 (s, 2H), 4.50 (d, J=1.8 Hz, 2H), 3.77 (s, 3H), 3.74-3.70 (m, 4H), 3.62 (s, 2H), 3.40-3.35 (m, 4H), 2.14 (s, 3H), 1.60 (d, J=7.1 Hz, 3H).

### Example 34. Synthesis of [2-chloro-4-[1-[5-[2-(morpholinomethyl)phenyl]-2-thieny]ethylamino]-5,7-dihydropyrrolo[3,4-d]pyrimidin-6-yl]-morpholino-methanone

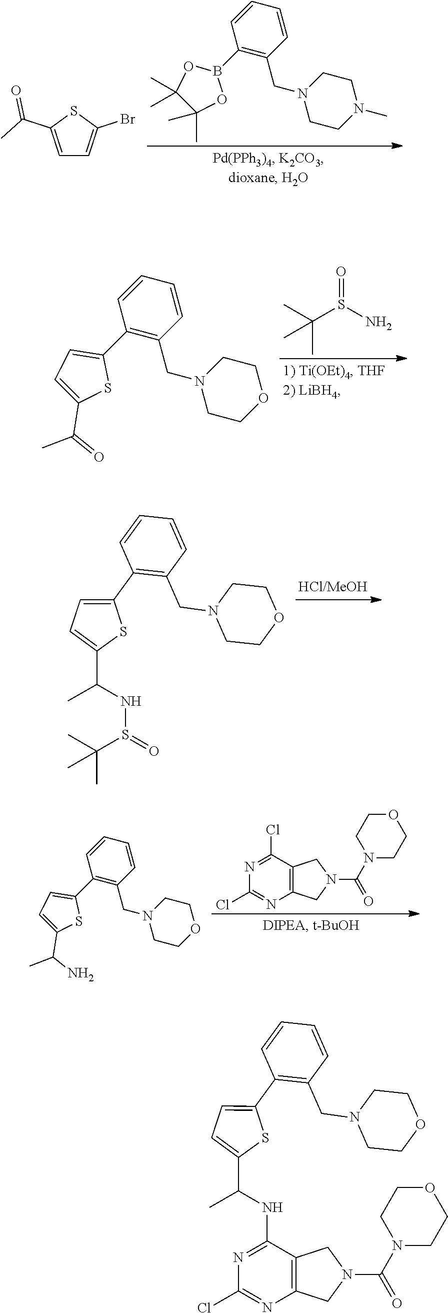

### Step 1.

1-[5-[2-(morpholinomethyl)phenyl]-2-thienyl]ethanone was synthesized in a manner similar to 1-[5-[2-[(4-methylpiperazin-1-yl)methyl]phenyl]-2-thienyl]ethenone except 1-methyl-4-[[2-(4,4,5,5-tetramethyl-1,3,2-dioxaborolan-2-yl)phenyl]methyl]piperazine was substituted with 4-[[2-(4,4,5,5-tetramethyl-1,3,2-dioxaborolan-2-yl)phenyl]methyl]morpholine. LCMS (ESI): m/z: [M+H] calculated for C₁₇H₂₀NO₂S: 302.11. found 302.1.

### Step 2.

2-methyl-N-[(1R)-1-[5-[2-(morpholinomethyl)phenyl]-2-thienyl]ethyl]propane-2-sulfonamide was synthesized in a manner similar to 2-methyl-N-[1-[5-[2-[(4-methylpiperazin-1-yl)methyl]phenyl]-2-thienyl]ethyl]propane-2-sulfinamide except 1-[5-[2-[(4-methylpiperazin-1-yl)methyl]phenyl]-2-thienyl]ethenone was substituted with 1-[5-[2-(morpholinomethyl)phenyl]-2-thienyl]ethenone. LCMS (ESI): m/z: [M+H] calculated for C₂₁H₃₁N₂O₂S₂: 407.17. found 407.2; ¹H NMR (400 MHz, CDCl₃) δ ppm 7.48-7.40 (m, 2H), 7.34-7.28 (m, 2H), 7.14 (d, J=3.2 Hz, 1H), 7.02 (d, J=3.6 Hz, 1H), 4.85 (m, J=6.2 Hz, 1H), 3.70 (s, 4H), 3.50 (s, 2H), 2.46 (s, 4H), 1.64 (d, J=6.4 Hz, 3H), 1.25 (s, 9H).

### Step 3.

1-[5-[2-(morpholinomethyl) phenyl]-2-thienyl]ethanamine was synthesized in a manner similar to 1-[5-[2-[(4-methylpiperazin-1-yl)methyl]phenyl]-2-thienyl]ethanamine except 2-methyl-N-[1-[5-[2-[(4-methylpiperazin-1-yl)methyl]phenyl]-2-thienyl]ethyl]-propane-2-sulfinamide was substituted with 2-methyl-N-[(1R)-1-[5-[2-(morpholinomethyl)phenyl]-2-thienyl]ethyl]-propane-2-sulfonamide. LCMS (ESI): m/z: [M+H] calculated for C₁₇H₂₃N20S: 303.15. found 303.1.

### Step 4.

[2-chloro-4-[1-[5-[2-(morpholinomethyl)phenyl]-2-thienyl]ethylamino]-5,7-dihydropyrrolo[3,4-d]pyrimidin-6-yl]-morpholino-methanone was synthesized in a manner similar to [2-chloro-4-[1-[5-[2-[(4-methylpiperazin-1-yl)methyl]phenyl]-2-thienyl]ethylamino]-5,7-dihy dropyrrolo[3,4-d]pyrimidin-6-yl]-morpholino-methanone except 1-[5-[2-[(4-methylpiperazin-1-yl)methyl]phenyl]-2-thienyl]ethanamine was substituted with 1-[5-[2-(morpholinomethyl) phenyl]-2-thienyl]ethanamine. LCMS (ESI): m/z: [M+H] calculated for C₂₈H₃₄ClN₆O₃S: 569.20. found 569.2; H NMR (400 MHz, METHANOL-d₄) δ ppm 7.45-7.41 (m, 1H), 7.41-7.36 (m, 1H), 7.33-7.25 (m, 2H), 7.07 (d, J=3.6 Hz, 1H), 7.03 (d, J=3.6 Hz, 1H), 5.76-5.68 (m, 1H), 4.59 (s, 4H), 3.74-3.67 (m, 4H), 3.62-3.55 (m, 4H), 3.49 (s, 2H), 3.37-3.33 (m, 4H), 2.37 (s, 4H), 1.70 (d, J=6.6 Hz, 3H).

### Example 35. Synthesis of [1-[[(1R)-1-[3-amino-5-(trifluoromethyl)phenyl]ethyl]amino]-4-chloro-5,7-dihydropyrrolo[3,4-d]pyridazin-6-yl]-morpholino-methanone

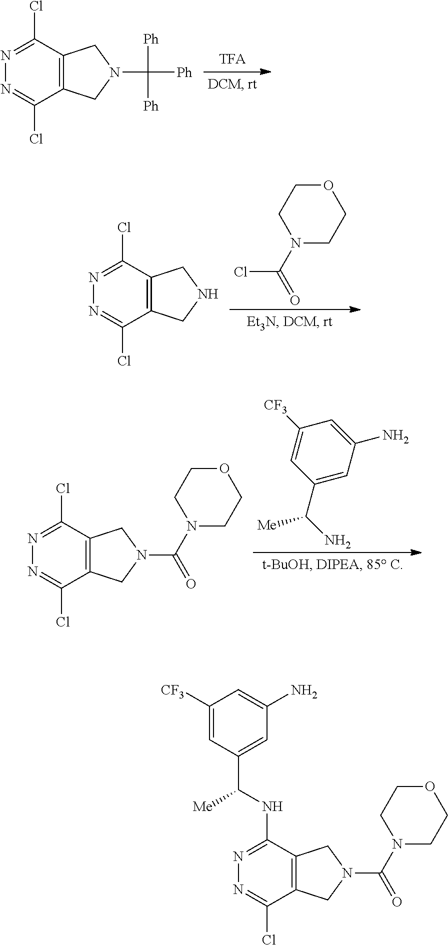

### Step 1.

To a mixture of 1,4-dichloro-6-trityl-5,7-dihydropyrrolo[3,4-d]pyridazine (1.5 g, 3.47 mmol) in DCM (15 mL) was added TFA (2.57 mL, 34.7 mmol) at 0° C. The mixture was stirred at rt for 4 h, then the solvent was concentrated under reduced pressure to give 1,4-dichloro-6,7-dihydro-5H-pyrrolo[3,4-d]pyridazine TFA salt (1 g).

### Step 2.

To a mixture of morpholine-4-carbonyl chloride (1.15 mL, 9.87 mmol) in DCM (10 mL) at 0° C. was added TEA (2.06 mL, 14.8 mmol) and 1,4-dichloro-6,7-dihydro-5H-pyrrolo[3,4-d]pyridazine (1 g, 3.3 mmol). The mixture was stirred at rt for 5 h, the solvent was concentrated under reduced pressure and the residue was purified by column chromatography to give (1,4-dichloro-5,7-dihydropyrrolo[3,4-d]pyridazin-6-yl)-morpholino-methanone (100 mg, 10% yield). LCMS (ESI): m/z: [M+H] calculated for C₁₁H₁₃Cl₂N₄O₂: 303.0. found 303.1; H NMR (400 MHz, METHANOL-d₄) δ ppm 4.95 (s, 4H), 3.75-3.70 (m, 4H), 3.42-3.37 (m, 4H).

### Step 3.

To a mixture of (1,4-dichloro-5,7-dihydropyrrolo[3,4-d]pyridazin-6-yl)-morpholino-methanone (100 mg, 0.33 mmol) in t-BuOH (1 mL) was added 3-[(1R)-1-aminoethyl]-5-(trifluoromethyl)aniline (142 mg, 0.69 mmol) and DIPEA (289 μL, 1.65 mmol) at rt under an atmosphere of N₂. The mixture was heated to 85° C. in a crimped vial and stirred for 12 h. The solvent was concentrated under reduced pressure and the residue was purified by prep-HPLC to give [1-[[(1R)-1-[3-amino-5-(trifluoromethyl)phenyl]ethyl]amino]-4-chloro-5,7-dihydropyrrolo[3,4-d]pyridazin-6-yl]-morpholino-methanone (6 mg, 4% yield). LCMS (ESI): m/z: [M+H] calculated for C₂₀H₂₃ClF₃N6O₂: 471.1. found 471.2; ¹H NMR (400 MHz, METHANOL-d₄) δ ppm 6.93 (d, J=5.6 Hz, 2H), 6.77 (s, 1H), 5.31-5.24 (m, 1H), 4.80-4.76 (m, 4H), 3.76-3.71 (m, 4H), 3.41-3.36 (m, 4H), 1.56 (d, J=7.0 Hz, 3H).

### Example 36. Synthesis of (R)—N-(1-(3-amino-5-(trifluoromethyl)phenyl)ethyl)-6-((6-aminopyridin-3-yl)sulfonyl)-2-methyl-6,7-dihydro-5H-pyrrolo[3,4-d]pyrimidin-4-amine

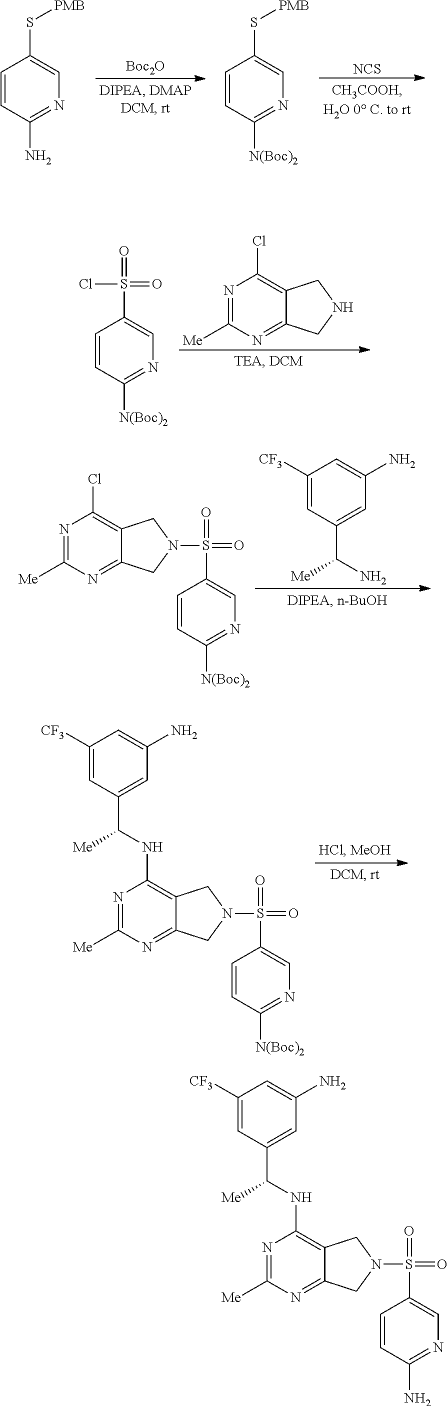

### Step 1.

To a mixture of 5-((4-methoxybenzyl)thio)pyridin-2-amine (3.5 g, 14.2 mmol), DMAP (1.74 g, 14.2 mmol), DIPEA (7.4 mL, 42.6 mmol) in DCM (120 mL) was add di-tert-butyl dicarbonate (18.6 g, 85.3 mmol). The mixture was purged with N₂ (×3), stirred at rt for 16 h, then extracted with EtOAc (30 mL×3). The combined organic layers were washed with H₂O (30 mL×2) and brine (30 mL×2), dried over anhydrous Na₂SO₄, and filtered. The solvent was concentrated under reduced pressure and the residue was purified by column chromatography to give tert-butyl N-tert-butoxycarbonyl-N-[5-[(4-methoxyphenyl)methylsulfanyl]-2-pyridyl]carbamate (2.5 g, 34% yield; note: 85% purity). LCMS (ESI): m/z: [M+H] calculated for C₂₃H₃₁N₂O₅S: 447.19. found: 447.1; H NMR (400 MHz, DMSO-d₆) δ ppm 8.32 (d, J=2.4 Hz, 1H), 7.82 (dd, J=8.4 Hz, 1H), 7.26 (m, 3H), 6.83 (d, J=8.8 Hz, 2H), 4.24 (s, 2H), 3.70 (s, 3H), 1.37 (s, 18H).

### Step 2.

To a mixture of tert-butyl N-tert-butoxycarbonyl-N-[5-[(4-methoxyphenyl)methylsulfanyl]-2-pyridyl]carbamate (2.0 g, 4.5 mmol) in AcOH (60 mL) and H₂O (15 mL) at 0° C. was added N-chlorosuccinimide (4.19 g, 31.4 mmol). The mixture was warmed to rt and stirred for 5 h, then extracted with EtOAc (30 mL×3). The combined organic layers were washed with H₂O (30 mL×2) and brine (30 mL×2), dried over anhydrous Na₂SO₄, and filtered. The solvent was concentrated under reduced pressure and the residue was purified by column chromatography to give tert-butyl N-tert-butoxycarbonyl-N-(5-chlorosulfonyl-2-pyridyl)carbamate (1.1 g, 38% yield; note: 60% purity). LCMS (ESI): m/z: [M+H] calculated for C₁₅H₂₂ClN₂O₆S: 393.08. found: 393.0.

### Step 3.

To a mixture of 4-chloro-2-methyl-6,7-dihydro-5H-pyrrolo[3,4-d]pyrimidine (80 mg, 0.47 mmol) in DCM (6 mL) at −30° C. was added TEA (197 μL, 1.42 mmol) dropwise, then tert-butyl N-tert-butoxycarbonyl-N-(5-chlorosulfonyl-2-pyridyl)carbamate, 60% purity (309 mg, 0.47 mmol) was added dropwise at −30° C. The mixture was warmed to rt and stirred for 1 h, then extracted with DCM (30 mL×3). The combined organic layers were washed with H₂O (30 mL×2) and brine (30 mL×2), dried over anhydrous Na₂SO₄, and filtered. The solvent was concentrated under reduced pressure and the residue was purified by prep-TLC to give tert-butyl N-tert-butoxycarbonyl-N-[5-[(4-chloro-2-methyl-5,7-dihydropyrrolo[3,4-d]pyrimidin-6-yl)sulfonyl]-2-pyridyl]carbamate (190 mg, 33% yield; note: 75% purity). LCMS (ESI): m/z: [M+H] calculated for C₂₂H₂₉ClN₅O₆S: 526.14. found: 526.2.

### Step 4.

A mixture of tert-butyl N-tert-butoxycarbonyl-N-[5-[(4-chloro-2-methyl-5,7-dihydropyrrolo[3,4-d]pyrimidin-6-yl)sulfonyl]-2-pyridyl]carbamate (170 mg, 0.24 mmol; note: 75% purity) (R)-3-(1-aminoethyl)-5-(trifluoromethyl)aniline (74 mg, 0.36 mmol), and DIPEA (127 μL, 0.73 mmol) in n-BuOH (16 mL) was purged with N₂ (×3), then heated to 110° C. and stirred for 3.5 h. The mixture was extracted with EtOAc (20 mL×3), and the combined organic layers were washed with H₂O (20 mL×2) and brine (20 mL×2), dried over anhydrous Na₂SO₄, and filtered. The solvent was concentrated under reduced pressure and the residue was purified by prep-TLC to give tert-butyl N-[5-[[4-[[(1R)-1-[3-amino-5-(trifluoromethyl)phenyl]ethyl]amino]-2-methyl-5,7-dihydropyrrolo[3,4-d]pyrimidin-6-yl]sulfonyl]-2-pyridyl]-N-tert-butoxycarbonyl-carbamate (100 mg, 49% yield). LCMS (ESI): m/z: [M+H] calculated for C₃₁H₃₉F₃N₇O₆S: 694.26. found: 694.2.

### Step 5.

A mixture of tert-butyl N-[5-[[4-[[(1R)-1-[3-amino-5-(trifluoromethyl)phenyl]ethyl]amino]-2-methyl-5,7-dihydropyrrolo[3,4-d]pyrimidin-6-yl]sulfonyl]-2-pyridyl]-N-tert-butoxycarbonyl-carbamate (90 mg, 87 μmol) in 4M HCl in MeOH (5 mL) was degassed with N₂ (×3), then stirred at rt for 1 h. The solvent was concentrated under reduced pressure and the residue was purified by prep-HPLC to give (R)—N-(1-(3-amino-5-(trifluoromethyl)phenyl)ethyl)-6-((6-aminopyridin-3-yl)sulfonyl)-2-methyl-6,7-dihydro-5H-pyrrolo[3,4-d]pyrimidin-4-amine (21 mg, 81% yield). LCMS (ESI): m/z: [M+H] calculated for C₂₁H₂₃F₃N₇O₂S: 494.15. found: 494.1; H NMR (400 MHz, DMSO-d₆) δ ppm 8.32 (s, 1H), 7.20 (d, J=8.4 Hz, 1H), 7.48 (d, J=8.0 Hz, 1H), 6.98 (s, 2H), 6.80 (s, 1H), 6.75 (s, 1H), 6.67 (s, 1H), 6.50 (d, J=9.2 Hz, 1H) 5.52 (br s, 2H), 5.23 (m, 1H), 4.29-4.34 (m, 4H), 2.29 (s, 3H), 1.39 (d, J=6.8 Hz, 3H).

### Example 37. Synthesis of (R)—N-(1-(3-amino-5-(trifluoromethyl)phenyl)ethyl)-2-methyl-6-(morpholinosulfonyl)-6,7-dihydro-5H-pyrrolo[3,4-d]pyrimidin-4-amine

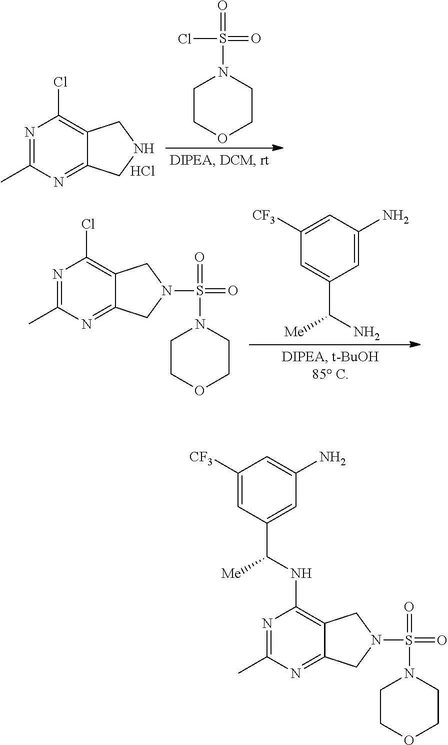 [SMILES: Cc1nc(Cl)c2c(n1)CNC2 ; Cl ; O=S(=O)(Cl)N1CCOCC1 ; Cc1nc(Cl)c2c(n1)CN(S(=O)(=O)N1CCOCC1)C2 ; C[C@@H](N)c1cc(N)cc(C(F)(F)F)c1 ; Cc1nc2c(c(N[C@H](C)c3cc(N)cc(C(F)(F)F)c3)n1)CN(S(=O)(=O)N1CCOCC1)C2]

### Step 1.

To a mixture of morpholine-4-sulfonyl chloride (901 mg, 4.85 mmol) in DCM (6 mL) was added DIPEA (507 μL, 2.91 mmol) and 4-chloro-2-methyl-6,7-dihydro-5H-pyrrolo[3,4-d]pyrimidine HCl salt (0.1 g, 0.5 mmol) in DCM (3 mL). The mixture was stirred at rt for 13 h, then MeOH (15 mL) was added and the solvent was removed under reduced pressure. The residue was triturated with MeOH (2.5 mL) and filtered. The solvent was concentrated under reduced pressure and the residue was purified by prep-HPLC to give 4-((4-chloro-2-methyl-5H-pyrrolo[3,4-d] pyrimidin-6(7H)-yl)sulfonyl)morpholine (0.07 g). LCMS (ESI): m/z: [M+H] calculated for C₁₁H₁₆ClN₃O₃S: 319.06. found 319.0; ¹H NMR (400 MHz, CDCl₃) δ ppm 4.68-4.75 (m, 4H), 3.74-3.81 (m, 4H), 3.28-3.35 (m, 4H), 2.74 (s, 3H).

### Step 2.

To a mixture of 4-((4-chloro-2-methyl-5H-pyrrolo[3,4-d]pyrimidin-6(7H)-yl)sulfonyl) morpholine (0.05 g, 0.16 mmol) and (R)-3-(1-aminoethyl)-5-(trifluoromethyl)aniline (32 mg, 0.16 mmol) in t-BuOH (5 mL) was added DIPEA (137 μL, 0.78 mmol). The mixture was heated to 85° C. in a crimped vial and stirred for 21 h. The solvent was concentrated under reduced pressure and the residue was purified by prep-HPLC to give (R)—N-(1-(3-amino-5-(trifluoromethyl)phenyl)ethyl)-2-methyl-6-(morpholinosulfonyl)-6,7-dihydro-5H-pyrrolo[3,4-d]pyrimidin-4-amine (16 mg, 15% yield). LCMS (ESI): m/z: [M+H] calculated for C₂₀H₂₆F₃N₆O₃S: 487.17. found 487.1; ¹H NMR (400 MHz, CDCl₃) δ ppm 6.99 (s, 1H), 6.81 (br s, 2H), 5.36 (br t, J=6.8 Hz, 1H), 4.66 (br d, J=6.2 Hz, 1H), 4.44-4.57 (m, 4H), 3.90 (br s, 2H), 3.68-3.79 (m, 4H), 3.20-3.30 (m, 4H), 2.51 (s, 3H), 1.58 (s, 3H).

### Example 38. Synthesis of (R)—N-(1-(3-amino-5-(trifluoromethyl)phenyl)ethyl)-2-methyl-6-((4-methylpiperazin-1-yl)sulfonyl)-6,7-dihydro-5H-pyrrolo[3,4-d]pyrimidin-4-amine

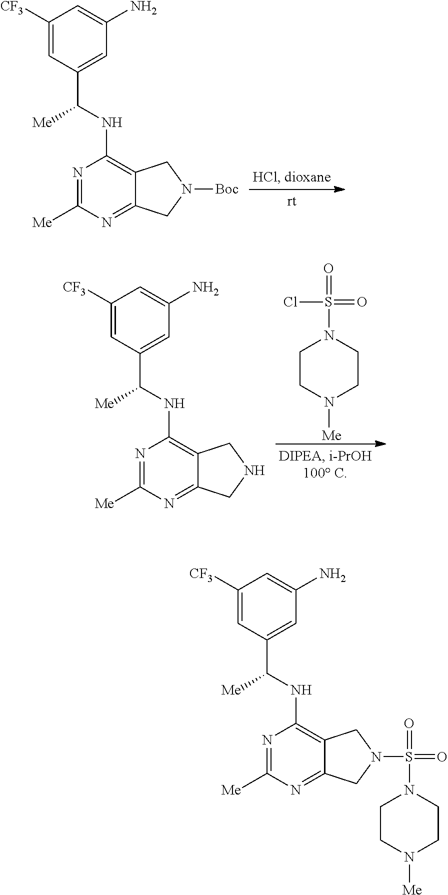 [SMILES: Cc1nc2c(c(N[C@H](C)c3cc(N)cc(C(F)(F)F)c3)n1)CN(C(=O)OC(C)(C)C)C2 ; Cc1nc2c(c(N[C@H](C)c3cc(N)cc(C(F)(F)F)c3)n1)CNC2 ; CN1CCN(S(=O)(=O)Cl)CC1 ; Cc1nc2c(c(N[C@H](C)c3cc(N)cc(C(F)(F)F)c3)n1)CN(S(=O)(=O)N1CCN(C)CC1)C2]

### Step 1.

A mixture of (R)-tert-butyl 4-((1-(3-amino-5-(trifluoromethyl)phenyl)ethyl)amino)-2-methyl-5H-pyrrolo[3,4-d]pyrimidine-6(7H)-carboxylate (150 mg, 0.34 mmol) in 4M HCl in MeOH (2 mL) was degassed and purged with N₂ (×3). The mixture was stirred at rt for 3 h, then the solvent was concentrated under reduced pressure to give (R)—N-(1-(3-amino-5-(trifluoromethyl)phenyl)ethyl)-2-methyl-6,7-dihydro-5H-pyrrolo[3,4-d]pyrimidin-4-amine (110 mg, 88% yield). LCMS (ESI): m/z: [M+H] calculated for C₁₋₆H₁₉F₃N₅: 338.15. found: 338.1.

### Step 2.

To a mixture of (R)—N-(1-(3-amino-5-(trifluoromethyl)phenyl)ethyl)-2-methyl-6,7-dihydro-5H-pyrrolo[3,4-d]pyrimidin-4-amine (110 mg, 0.33 mmol) in i-PrOH (2 mL) was added DIPEA (284 μL, 1.63 mmol) and 4-methylpiperazine-1-sulfonyl chloride (65 mg, 0.33 mmol). The mixture was heated to 100° C. in a crimped vial and stirred for 16 h. The mixture was concentrated under reduced pressure and the residue was diluted with H₂O (2 mL) and extracted with EtOAc (2 mL×3). The combined organic layers were washed with brine (1 mL), dried over Na₂SO₄, and filtered. The solvent was concentrated under reduced pressure and the residue was purified by prep-HPLC to give (R)—N-(1-(3-amino-5-(trifluoromethyl)phenyl)ethyl)-2-methyl-6-((4-methylpiperazin-1-yl)sulfonyl)-6,7-dihydro-5H-pyrrolo[3,4-d]pyrimidin-4-amine (75 mg, 46% yield). LCMS (ESI): m/z: [M+H] calculated for C₂₁H₂₉F₃N₇O₂S: 500.2. found: 500.2; ¹H NMR (400 MHz, CDCl₃) δ ppm 7.00 (s, 1H), 6.82 (s, 2H), 5.36 (s, 1H), 4.75 (s, 1H), 4.41-4.61 (m, 4H), 3.70 (s, 2H), 3.34-3.39 (m, 4H), 2.49-2.60 (m, 7H), 2.37 (s, 3H), 1.59 (s, 2H).

### Example 39. Synthesis of [2-chloro-4-[1-[5-[2-(methylaminomethyl)phenyl]-2-thienyl]ethylamino]-5,7-dihydropyrrolo[3,4-d] pyrimidin-6-yl]-morpholino-methanone

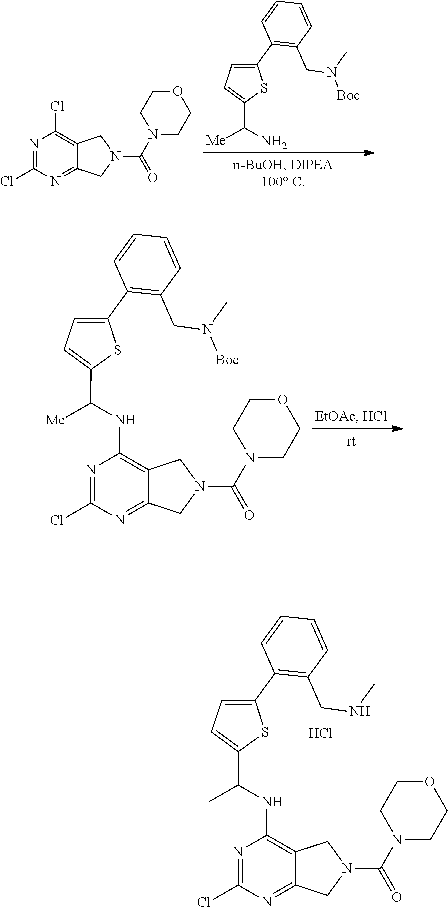

### Step 1.

Tert-butyl-N-[[2-[5-[1-[[2-chloro-6-(morpholine-4-carbonyl)-5,7-dihydropyrrolo[3,4-d]pyrimidin-4-yl]amino]ethyl]-2-thienyl]phenyl]methyl]-N-methyl-carbamate was synthesized in a manner similar to tert-butyl N-[[2-[4-[1-[[2-chloro-6-(morpholine-4-carbonyl)-5,7-dihydropyrrolo[3,4-d]pyrimidin-4-yl]amino]ethyl]thiazol-2-yl]phenyl]methyl]-N-methyl-carbamate except tert-butyl N-[[2-[3-(1-aminoethyl)phenyl]phenyl]methyl]-N-methyl-carbamate was substituted with tert-butyl-N-[[2-[5-(1-aminoethyl)-2-thienyl]phenyl]methyl]-N-methyl-carbamate. ¹H NMR (400 MHz, CDCl₃) δ ppm 7.35-7.38 (m, 2H), 7.24-7.30 (m, 2H), 7.00 (s, 1H), 6.84 (d, J=2.8 Hz, 1H), 5.72 (s, 1H), 5.02 (s, 1H), 4.53-4.67 (m, 6H), 3.71 (m, 4H), 3.33-3.40 (m, 4H), 2.74 (d, 3H), 1.73 (d, J=6.8, 3H), 1.46 (m, 9H).

A mixture of tert-butyl N-[[2-[5-[1-[[2-chloro-6-(morpholine-4-carbonyl)-5,7-dihydropyrrolo[3,4-d]pyrimidin-4-yl]amino]ethyl]-2-thienyl]phenyl]methyl]-N-methyl-carbamate (0.06 g, 0.1 mmol) in 4M HCl in EtOAc (1 mL) was stirred at rt for 1 h. The solvent was concentrated under reduced pressure and the residue was purified by prep-HPLC to give [2-chloro-4-[1-[5-[2-(methylaminomethyl)phenyl]-2-thienyl]ethylamino]-5,7-dihydropyrrolo[3,4-d]pyrimidin-6-yl]-morpholino-methanone HCl salt (15.2 mg, 28% yield). LCMS (ESI): m/z: [M+H] calculated for C₂₅H₂₉ClN₆O₂S: 513.18. found 513.2; ¹H NMR (400 MHz, METHANOL-d₄) δ ppm 7.56-7.60 (m, 1H), 7.48-7.53 (m, 3H), 7.14 (d, J=3.6 Hz, 1H), 7.00 (d, J=3.6 Hz, 1H), 5.75-5.8 (m, 1H), 4.65 (d, J=7.2 Hz, 4H), 4.34 (s, 2H), 3.72 (t, J=4.8 Hz, 4H), 3.37 (t, J=4.8 Hz, 4H), 2.65 (s, 3H), 3.68 (s, 3H), 1.74 (m, 3H).

### Example 40. Synthesis of [2-chloro-4-[1-[3-hydroxy-5-(trifluoromethyl)phenyl]ethylamino]-5,7-dihydropyrrolo[3,4-d]pyrimidin-6-yl]-morpholino-methanone

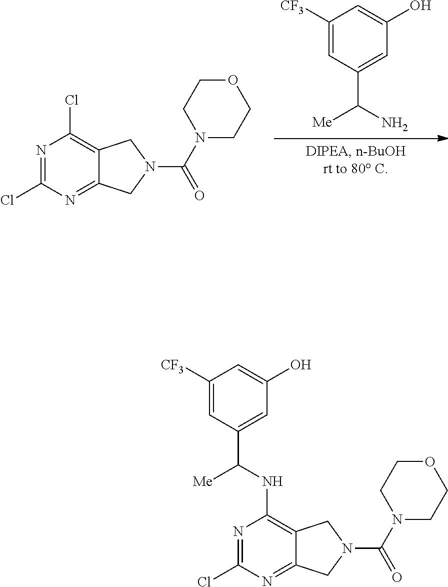

### Step 1.

To a mixture of (2,4-dichloro-5,7-dihydropyrrolo[3,4-d]pyrimidin-6-yl)-morpholino-methanone (100 mg, 0.33 mmol) and 3-(1-aminoethyl)-5-(trifluoromethyl)phenol (68 mg, 0.33 mmol) in n-BuOH (1 mL) was added DIPEA (115 μL, 0.66 mmol). The mixture was heated to 80° C. and stirred for 3 h. The mixture was filtered, the solvent was concentrated under reduced pressure and the residue was purified by prep-HPLC to give [2-chloro-4-[1-[3-hydroxy-5-(trifluoromethyl)phenyl]ethylamino]-5,7-dihydropyrrolo[3,4-d]pyrimidin-6-yl]-morpholino-methanone (20 mg, 13% yield). LCMS (ESI): m/z: [M−H] calculated for C₂₀H₂₂ClF₃N₅O₃: 472.13. found 472.2; ¹H NMR (400 MHz, DMSO-d₆) δ ppm 10.18-10.04 (br m, 1H), 8.14 (br d, J=7.6 Hz, 1H), 7.16 (s, 1H), 7.03 (s, 1H), 6.90 (s, 1H), 5.32-5.18 (m, 1H), 4.64-4.45 (m, 4H), 3.61 (d, J=4.4 Hz, 4H), 3.24 (d, J=4.0 Hz, 4H), 1.47 (d, J=7.2 Hz, 3H).

### Example 41. Synthesis of [4-[1-(3-amino-5-ethyl-phenyl)ethylamino]-2-chloro-5,7-dihydropyrrolo[3,4-d]pyrimidin-6-yl]-morpholino-methanone

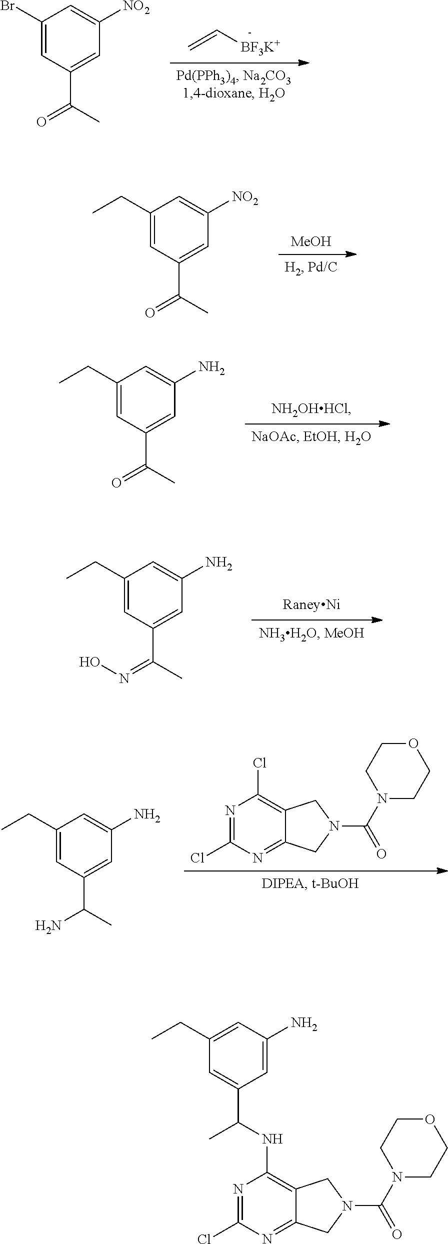

### Step 1.

To a mixture of 1-(3-bromo-5-nitro-phenyl)ethanone (1.3 g, 5.33 mmol) in 1,4-dioxane (12 mL) and H₂O (3 mL) under an atmosphere of N₂ was added potassium trifluoro(vinyl)borate (785 mg, 5.86 mmol), Pd(PPh₃)₄(616 mg, 0.53 mmol) and Na₂CO₃ (1.13 g, 10.7 mmol). The mixture was heated to 90° C. and stirred for 10 h. H₂O (10 mL) was added and the mixture was extracted with EtOAc (10 mL×3). The combined organic layers were washed with brine (20 mL), dried over Na₂SO₄, and filtered. The solvent was concentrated under reduced pressure and the residue was purified by column chromatography to give 1-(3-nitro-5-vinyl-phenyl)ethanone (500 mg, 49% yield). ¹H NMR (400 MHz, CDCl₃) δ ppm 8.64 (s, 1H), 8.45 (s, 1H), 8.28 (s, 1H), 6.83 (dd, J=17.6, 11.2 Hz, 1H), 6.00 (d, J=17.6 Hz, 1H), 5.56 (d, J=10.8 Hz, 1H), 2.71 (d, J=2.0 Hz, 3H).

### Step 2.

To a mixture of 1-(3-nitro-5-vinyl-phenyl)ethanone (250 mg, 1.31 mmol) in MeOH (2 mL) was added 10% wt. Pd/C (1.54 g, 1.31 mmol). The mixture was stirred under at atmosphere of H₂ (15 psi) for 3 h. The mixture was filtered, the solvent was concentrated under reduced pressure and the residue was purified by prep-TLC to give 1-(3-amino-5-ethyl-phenyl)ethanone (110 mg, 52% yield). ¹H NMR (400 MHz, CDCl₃) δ ppm 7.19 (s, 1H), 7.10 (s, 1H), 6.74 (s, 1H), 3.75 (br s, 2H), 2.63 (q, J=7.6 Hz, 2H), 2.58-2.54 (m, 3H), 1.56 (s, 1H), 1.24 (t, J=7.6 Hz, 3H).

### Step 3.

To a mixture of NH₂OH.HCl (64 mg, 0.92 mmol) in EtOH (1 mL) and H₂O (0.5 mL) was added NaOAc (126 mg, 1.53 mmol) and 1-(3-amino-5-ethyl-phenyl)ethanone (100 mg, 0.61 mmol). The mixture was heated to 80° C. and stirred for 5 h. The solvent was concentrated under reduced pressure and the residue was purified by prep-TLC to give 1-(3-amino-5-ethyl-phenyl)ethanone oxime (100 mg, 92% yield). H NMR (400 MHz, CDCl₃) δ ppm 6.84 (s, 1H), 6.76 (s, 1H), 6.58 (s, 1H), 4.91-4.27 (m, 2H), 2.64-2.54 (m, 2H), 2.34-2.21 (m, 3H), 1.23 (t, J=7.6 Hz, 3H).

### Step 4.

To a mixture of 1-(3-amino-5-ethyl-phenyl)ethanone oxime (100 mg, 0.56 mmol) in MeOH (0.5 mL) was added 30% aqueous NH₃ (72 μL, 0.56 mmol) and Raney-Ni (48 mg, 0.56 mmol). The mixture was stirred at rt for 4 h under an atmosphere of H₂ (15 psi). The mixture was filtered and the filtrated was concentrated under reduced pressure to give 3-(1-aminoethyl)-5-ethyl-aniline (150 mg), which was used into the next step without further purification. LCMS (ESI): m/z: [M+H] calculated for C₁₀H₁₇N₂: 165.13. found 165.3.

### Step 5.

To a mixture of (2,4-dichloro-5,7-dihydropyrrolo[3,4-d]pyrimidin-6-yl)-morpholino-methanone (83 mg, 0.27 mmol) in t-BuOH (1 mL) was added DIPEA (447 μL, 2.74 mmol) and 3-(1-aminoethyl)-5-ethyl-aniline (90 mg, 0.55 mmol). The mixture was heated to 50° C. and stirred for 1 h, then the solvent was concentrated under reduced pressure and the residue was purified by prep-HPLC to give [4-[1-(3-amino-5-ethyl-phenyl)ethylamino]-2-chloro-5,7-dihydropyrrolo[3,4-d]pyrimidin-6-yl]-morpholino-methanone (7 mg, 3% yield). LCMS (ESI): m/z: [M+H] calculated for C₂₁H₂₈ClN₆O₂: 431.19. found 431.2; H NMR (400 MHz, METHANOL-d₄) δ ppm 6.61 (s, 1H), 6.58 (s, 1H), 6.49 (s, 1H), 5.30 (m, 1H), 4.58 (d, J=14.0 Hz, 4H), 3.78-3.67 (m, 4H), 3.38-3.33 (m, 4H), 2.53 (q, J=7.6 Hz, 2H), 1.52 (d, J=7.2 Hz, 3H), 1.19 (t, J=7.6 Hz, 3H).

### Example 42. Synthesis of N-[(1R)-1-[3-amino-5-(trifluoromethyl)phenyl]ethyl]-6-(5-bromo-2-pyridyl)-2-methyl-7,8-dihydro-5H-pyrido[4,3-d]pyrimidin-4-amine

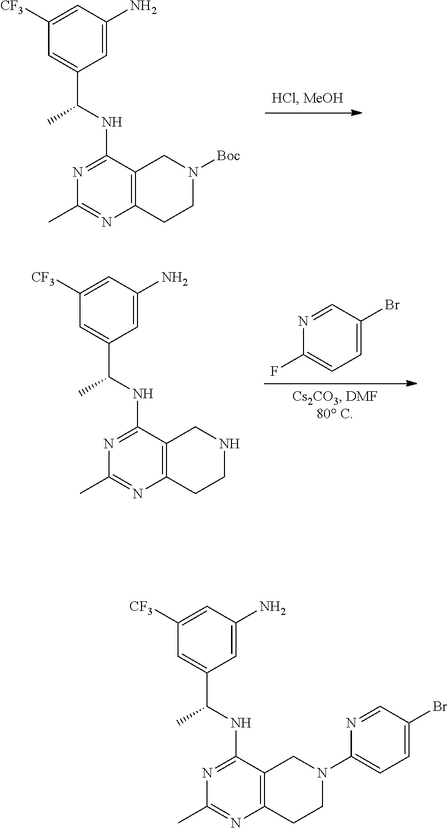

### Step 1.

To tert-butyl 4-[[(1R)-1-[3-amino-5-(trifluoromethyl)phenyl]ethyl]amino]-2-methyl-7,8-dihydro-5H-pyrido[4,3-d]pyrimidine-6-carboxylate (0.25 g, 0.55 mmol) was added HCl in MeOH (10 mL), and the mixture was stirred at rt for 1h. The solvent was concentrated under reduced pressure to give N-[(1R)-1-[3-amino-5-(trifluoromethyl)phenyl]ethyl]-2-methyl-5,6,7,8-tetrahydropyrido[4,3-d]pyrimidin-4-amine HCl salt (0.22 g). ¹H NMR (400 MHz, METHANOL-d₄) δ ppm 7.89 (d, J=10.8 Hz, 2H), 7.60 (s, 1H), 5.81-5.67 (m, 1H), 4.40 (d, J=16.0 Hz, 1H), 4.29 (d, J=16.0 Hz, 1H), 3.64 (t, J=6.2 Hz, 2H), 3.19-3.10 (m, 2H), 2.55 (s, 3H), 1.73 (d, J=7.1 Hz, 3H).

### Step 2.

To a mixture of N-[(1R)-1-[3-amino-5-(trifluoromethyl)phenyl]ethyl]-2-methyl-5,6,7,8-tetrahydropyrido[4,3-d]pyrimidin-4-amine HCl salt (0.22 g, 0.57 mmol) in DMF (3 mL) was added 5-bromo-2-fluoro-pyridine (200 mg, 1.13 mmol) and Cs₂CO₃ (1.11 g, 3.40 mmol). The mixture was heated to 80° C. and stirred for 13 h. The solvent was concentrated under reduced pressure and the residue was purified by prep-HPLC to give N-[(1R)-1-[3-amino-5-(trifluoromethyl)phenyl]ethyl]-6-(5-bromo-2-pyridyl)-2-methyl-7,8-dihydro-5H-pyrido[4,3-d]pyrimidin-4-amine HCO₂H salt (43 mg, 14% yield). LCMS (ESI): m/z: [M+H] calculated for C₂₂H₂₃BrF₃N₆: 507.1. found 507.1; ¹H NMR (400 MHz, METHANOL-d₄) δ ppm 8.38 (s, 1H), 8.20 (d, J=2.5 Hz, 1H), 7.70 (dd, J=9.1, 2.5 Hz, 1H), 6.95 (dd, J=9.5, 3.2 Hz, 3H), 6.80 (s, 1H), 5.52 (d, J=7.0 Hz, 1H), 4.46-4.31 (m, 2H), 4.00-3.83 (m, 2H), 2.82 (t, J=5.3 Hz, 2H), 2.41 (s, 3H), 1.60 (d, J=7.0 Hz, 3H).

### Example 186. Synthesis of (R)—N-(1-(3-amino-5-(trifluoromethyl)phenyl)ethyl)-2-chloro-6-(3-morpholinopyridin-2-yl)-5,6,7,8-tetrahydropyrido[4,3-d]pyrimidin-4-amine

(R)—N-(1-(3-amino-5-(trifluoromethyl)phenyl)ethyl)-2-chloro-6-(3-morpholinopyridin-2-yl)-5,6,7,8-tetrahydropyrido[4,3-d]pyrimidin-4-amine was synthesized in the manner similar to Example 42.

| Example | | mass |
|---|---|---|
| # | Structure | found |
| Example 186. | 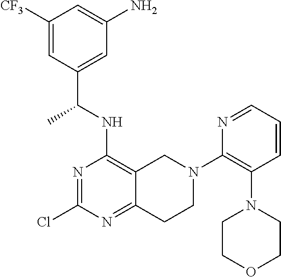 [SMILES: C[C@@H](Nc1nc(Cl)nc2c1CN(c1ncccc1N1CCOCC1)CC2)c1cc(N)cc(C(F)(F)F)c1] | 534.1 |

### Example 43. Synthesis of [4-[[(1R)-1-[3-amino-5-(trifluoromethyl)phenyl]ethyl]amino]-2-methyl-pyrrolo[2,1-f][1,2,4]triazin-6-yl]-(3-hydroxyazetidin-1-yl)methanone

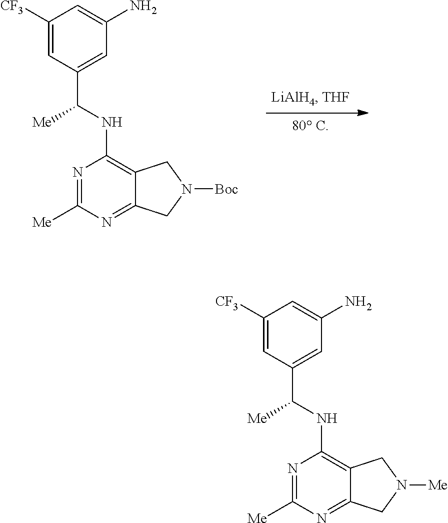

### Step 1.

To a mixture of tert-butyl 4-[[(1R)-1-[3-amino-5-(trifluoromethyl)phenyl]ethyl]amino]-2-methyl-5,7-dihydropyrrolo[3,4-d]pyrimidine-6-carboxylate (100 mg, 0.23 mmol) in THF (1 mL) was added LiAlH₄ (13 mg, 0.34 mmol). The mixture was heated to 80° C. and stirred for 1 h. The mixture was cooled to 15° C. then H₂O (3 mL) was added and the mixture extracted with EtOAc (5 mL×3). The combined organic layers were washed with brine (5 mL), dried over Na₂SO₄, and filtered. The solvent was concentrated under reduced pressure and the residue was purified by prep-HPLC to give [4-[[(1R)-1-[3-amino-5-(trifluoromethyl)phenyl]ethyl]amino]-2-methyl-pyrrolo[2,1-f][1,2,4]triazin-6-yl]-(3-hydroxyazetidin-1-yl)methanone (36 mg, 43% yield). LCMS (ESI): m/z: [M+H] calculated for C₁₇H₂₀F₃N₅: 352.2. found 352.1; ¹H NMR (400 MHz, METHANOL-d₄) δ ppm 8.41-8.22 (m, 1H), 6.90 (d, J=5.4 Hz, 2H), 6.79 (s, 1H), 5.39 (d, J=7.3 Hz, 1H), 4.11-4.01 (m, 4H), 2.78 (s, 3H), 2.40 (s, 3H), 1.52 (d, J=6.8 Hz, 3H).

### Example 44. Synthesis of N-[(1R)-1-[3-amino-5-(trifluoromethyl)phenyl]ethyl]-6-(5-bromo-2-pyridyl)-2-methyl-5,7-dihydropyrrolo[3,4-d]pyrimidin-4-amine

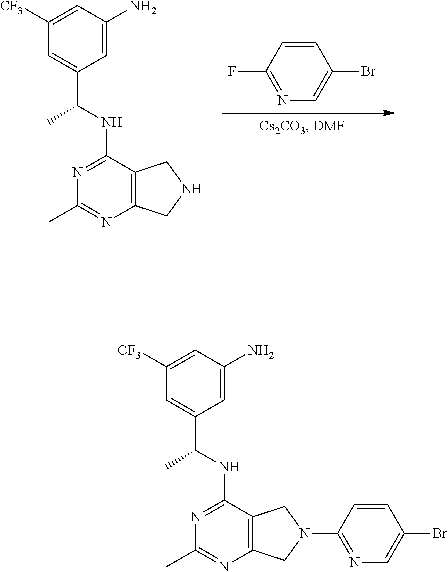

### Step 1.

To a mixture of N-[(1R)-1-[3-amino-5-(trifluoromethyl)phenyl]ethyl]-2-methyl-6,7-dihydro-5H-pyrrolo[3,4-d]pyrimidin-4-amine HCl salt (128 mg, 0.38 mmol) and 5-bromo-2-fluoro-pyridine (200 mg, 1.14 mmol) in DMF (3 mL) was added Cs₂CO₃ (618 mg, 1.90 mmol). The mixture was heated to 85° C. and stirred for 5 h, and then filtered. The solvent was concentrated under reduced pressure and the residue was purified by prep-HPLC to give N-[(1R)-1-[3-amino-5-(trifluoromethyl)phenyl]ethyl]-6-(5-bromo-2-pyridyl)-2-methyl-5,7-dihydropyrrolo[3,4-d]pyrimidin-4-amine (44 mg, 23% yield). LCMS (ESI): m/z: [M+H] calculated for C₂₁H₂₁BrF₃N₆: 493.09. found 493.1; ¹H NMR (400 MHz, METHANOL-d₄) δ ppm 8.17 (s, 1H), 7.71 (dd, J=8.9, 2.4 Hz, 1H), 6.96-6.90 (m, 2H), 6.81-6.77 (m, 1H), 6.61-6.57 (m, 1H), 5.49-5.41 (m, 1H), 4.65-4.61 (m, 1H), 4.56 (d, J=4.4 Hz, 4H), 2.43-2.41 (m, 3H), 1.57-1.54 (m, 3H).

### Example 45. Synthesis of [4-[[(1R)-1-[3-amino-5-(trifluoromethyl)phenyl]ethyl]amino]-2-methyl-6,7-dihydro-5H-cyclopenta[d]pyrimidin-6-yl]-morpholino-methanone

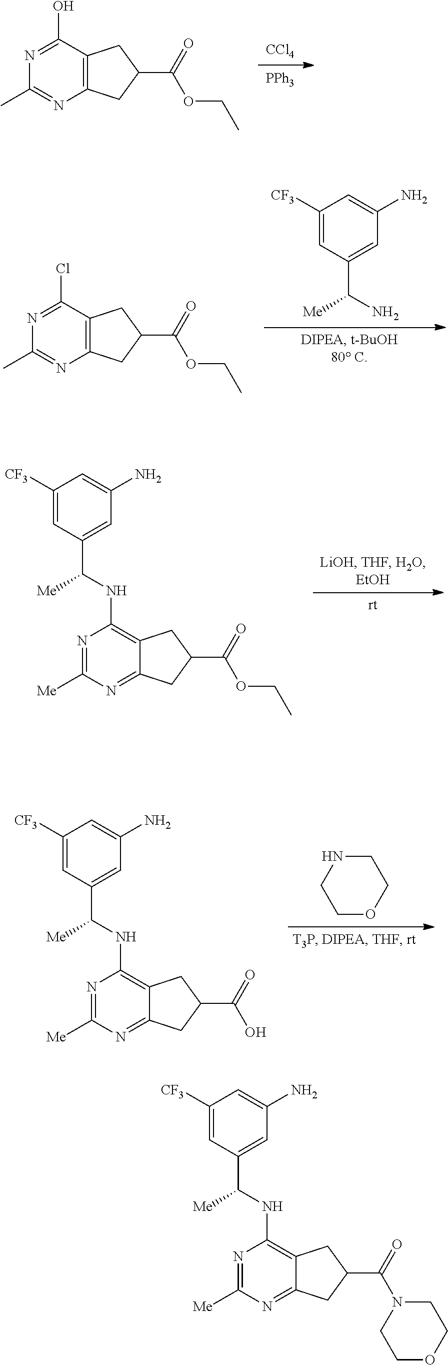

### Step 1.

To a mixture of ethyl 4-hydroxy-2-methyl-6,7-dihydro-5H-cyclopenta[d]pyrimidine-6-carboxylate (500 mg, 2.25 mmol) in DCE (5 mL) was added CCl₄ (649 μL, 6.75 mmol) and Ph₃P (1.18 g, 4.50 mmol). The mixture was heated to 70° C. and stirred for 2 h, then poured into H₂O (3 mL), and extracted with EtOAc (2 mL×3). The combined organic layers were washed with brine (1 mL), dried over Na₂SO₄, and filtered. The solvent was concentrated under reduced pressure and the residue was purified by column chromatography to give ethyl 4-chloro-2-methyl-6,7-dihydro-5H-cyclopenta[d]pyrimidine-6-carboxylate (400 mg, 74% yield). LCMS (ESI): m/z: [M+H] calculated for C₁H₁₄ClN₂O₂: 241.1. found 241.0.

### Step 2.

To a mixture of ethyl 4-chloro-2-methyl-6,7-dihydro-5H-cyclopenta[d]pyrimidine-6-carboxylate (200 mg, 0.83 mmol) in t-BuOH (0.2 mL) under an atmosphere of N₂ was added 3-[(1R)-1-aminoethyl]-5-(trifluoromethyl)aniline (170 mg, 0.83 mmol) and DIPEA (434 μL, 2.49 mmol). The mixture was heated to 80° C. and stirred for 20 h, then poured into H₂O (1 mL), and extracted with EtOAc (1 mL×3). The combined organic layers were washed with brine (1 mL), dried over Na₂SO₄, and filtered. The solvent was concentrated under reduced pressure and the residue was purified by prep-TLC to give ethyl 4-[[(1R)-1-[3-amino-5-(trifluoromethyl)phenyl]ethyl]amino]-2-methyl-6,7-dihydro-5H-cyclopenta[d]pyrimidine-6-carboxylate (170 mg, 50% yield). LCMS (ESI): m/z: [M+H] calculated for C₂₀H₂₄F₃N₄O₂: 409.2. found 409.2; ¹H NMR (400 MHz, CDCl₃) δ ppm 6.93 (s, 1H), 6.75 (d, J=15.2 Hz, 1H), 6.74-6.67 (m, 1H), 5.43-5.20 (m, 1H), 4.57-4.26 (m, 1H), 4.18-4.07 (m, 2H), 3.35-3.21 (m, 1H), 3.18-3.01 (m, 2H), 2.98-2.77 (m, 2H), 2.41 (s, 3H), 1.48 (t, J=6.8 Hz, 3H).

### Step 3.

To a mixture of ethyl 4-[[(1R)-1-[3-amino-5-(trifluoromethyl)phenyl]ethyl]amino]-2-methyl-6,7-dihydro-5H-cyclopenta[d]pyrimidine-6-carboxylate (170 mg, 0.42 mmol) in THF (0.8 mL) was added LiOH.H₂O (18 mg, 0.42 mmol), H₂O (0.6 mL) and EtOH (0.4 mL). The mixture was stirred at rt for 1 h, then concentrated under reduced pressure to give 4-[[(1R)-1-[3-amino-5-(trifluoromethyl)phenyl]ethyl]amino]-2-methyl-6,7-dihydro-5H-cyclopenta[d]pyrimidine-6-carboxylic acid (150 mg, 95% yield), which was used directly in the next step. LCMS (ESI): m/z: [M+H] calculated for C₁₈H₂₀F₃N₄O₂: 381.1. found 381.2; ¹H NMR (400 MHz, CDCl₃) δ ppm 6.93-6.91 (m, 1H), 6.70 (m, 1H), 6.67 (m, 1H), 5.34-5.29 (m, 1H), 2.93 (m, 2H), 2.91-2.89 (m, 1H), 2.86-2.85 (m, 2H), 2.34-2.33 (m, 3H), 1.48-1.44 (m, 3H).

### Step 4.

[4-[[(1R)-1-[3-amino-5-(trifluoromethyl)phenyl]ethyl]amino]-2-methyl-6,7-dihydro-5H-cyclopenta[d]pyrimidin-6-yl]-morpholino-methanone was synthesized in a manner similar to [4-[[(1R)-1-[3-amino-5-(trifluoromethyl)phenyl]ethyl]amino]thieno[3,2-d]pyrimidin-6-yl]-morpholino-methanone except 4-[[(1R)-1-[3-amino-5-(trifluoromethyl)phenyl]ethyl]amino]thieno[3,2-d] pyrimidine-6-carboxylic acid was substituted with 4-[[(1R)-1-[3-amino-5-(trifluoromethyl)phenyl]ethyl]amino]-2-methyl-6,7-dihydro-SH-cyclopenta[d]pyrimidine-6-carboxylic acid. LCMS (ESI): m/z: [M+H] calculated for C22H₂₇F₃N₅O₂: 450.2. found 450.2; ¹H NMR (400 MHz, METHANOL-d₄) δ ppm 6.92 (m, 1H), 6.89 (m, 1H), 6.77 (s, 1H), 5.41-5.37 (s, 1H), 3.71-3.67 (s, 2H), 3.67-3.66 (m, 4H), 3.63-3.61 (m, 2H), 3.12-3.11 (m, 2H), 3.05-3.03 (m, 2H), 3.01-2.92 (m, 1H), 2.35 (s, 3H), 1.52-1.50 (m, 3H).

### Example 46. Synthesis of (R)—N-(1-(3-amino-5-(trifluoromethyl)phenyl)ethyl)-2-methyl-6-(methylsulfonyl)-6,7-dihydro-5H-pyrrolo[3,4-d]pyrimidin-4-amine

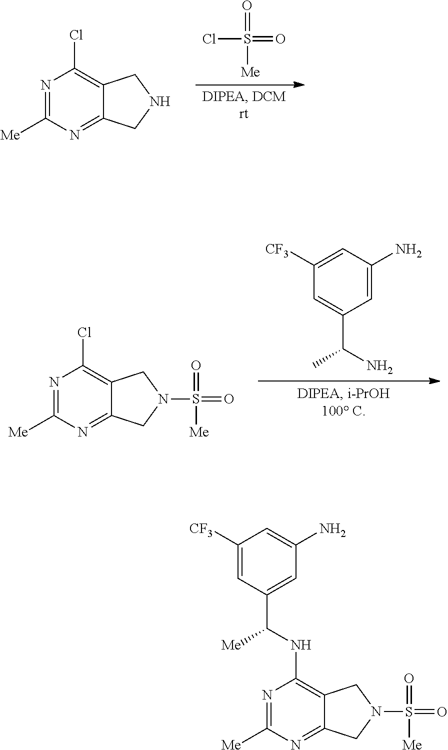

### Step 1.

To a mixture of 4-chloro-2-methyl-6,7-dihydro-5H-pyrrolo[3,4-d]pyrimidine (100 mg, 0.59 mmol) and DIPEA (308 μL, 1.77 mmol) in DCM (3 mL) at 0° C. was added methanesulfonyl chloride (55 μL, 0.71 mmol). The mixture was allowed to warm to rt and stirred for 3h. The mixture was diluted with DCM (6 mL), washed with brine (1 mL), dried over Na₂SO₄, filtered, and concentrated under reduced pressure to give 4-chloro-2-methyl-6-(methylsulfonyl)-6,7-dihydro-5H-pyrrolo[3,4-d]pyrimidine (135 mg, 92% yield). LCMS (ESI): m/z: [M+H] calculated for C₈H₁₁ClN₃02S: 248.0. found: 248.1.

### Step 2.

(R)—N-(1-(3-amino-5-(trifluoromethyl)phenyl)ethyl)-2-methyl-6-(methylsulfonyl)-6,7-dihydro-5H-pyrrolo[3,4-d]pyrimidin-4-amine was synthesized in a similar manner to (R)—N-(1-(3-amino-5-(trifluoromethyl)phenyl)ethyl)-2-methyl-6-(morpholinosulfonyl)-6,7-dihydro-5H-pyrrolo[3,4-d]pyrimidin-4-amine except 4-((4-chloro-2-methyl-5H-pyrrolo[3,4-d]pyrimidin-6(7H)-yl)sulfonyl) morpholine was substituted with 4-chloro-2-methyl-6-(methylsulfonyl)-6,7-dihydro-5H-pyrrolo[3,4-d]pyrimidine. LCMS (ESI): m/z: [M+H] calculated for C₁₇H₂₁F₃N₅O₂S: 416.13. found: 416.1; ¹H NMR (400 MHz, CDCl₃) δ ppm 6.99 (s, 1H), 6.82 (m, 2H), 5.38 (m, 1H), 4.66-4.77 (br s, 1H), 4.52 (s, 4H), 3.91 (br s, 2H), 2.89 (s, 3H), 2.47-2.55 (m, 3H), 1.58 (s, 3H).

### Example 47. Synthesis of N-[(1R)-1-[3-amino-5-(trifluoromethyl)phenyl]ethyl]-2-methyl-6-tetrahydropyran-4-ylsulfonyl-5,7-dihydropyrrolo[3,4-d]pyrimidin-4-amine

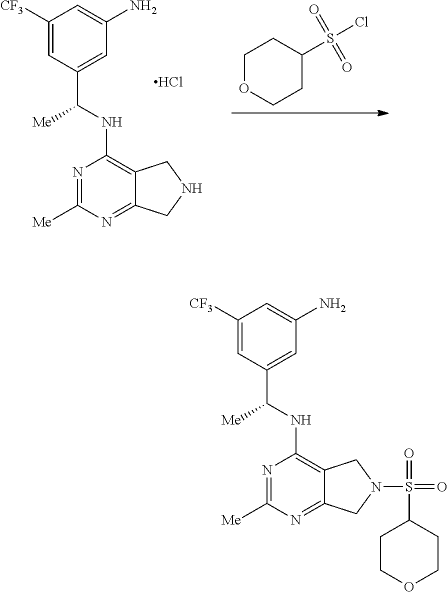 [SMILES: Cc1nc2c(c(N[C@H](C)c3cc(N)cc(C(F)(F)F)c3)n1)CNC2 ; Cl ; O=S(=O)(Cl)C1CCOCC1 ; Cc1nc2c(c(N[C@H](C)c3cc(N)cc(C(F)(F)F)c3)n1)CN(S(=O)(=O)C1CCOCC1)C2]

### Step 1.

To a mixture of N-[(1R)-1-[3-amino-5-(trifluoromethyl)phenyl]ethyl]-2-methyl-6,7-dihydro-5H-pyrrolo[3,4-d]pyrimidin-4-amine HCl salt (135 mg, 0.36 mmol) and tetrahydropyran-4-sulfonyl chloride (67 mg, 0.36 mmol) in MeCN (0.9 mL) and 1,4-dioxane (0.6 mL) was added TEA (151 μL, 1.08 mmol). The mixture was stirred at rt for 30 min, then concentrated under reduced pressure. The residue was diluted with H₂O (20 mL) and extracted with EtOAc (10 mL×3). The combined organic layers were washed with brine (20 mL), dried over Na₂SO₄, and filtered. The solvent was concentrated under reduced pressure and the residue was purified by prep-HPLC to give N-[(1R)-1-[3-amino-5-(trifluoromethyl)phenyl]ethyl]-2-methyl-6-tetrahydropyran-4-ylsulfonyl-5,7-dihydropyrrolo[3,4-d]pyrimidin-4-amine (90 mg, 51% yield). LCMS (ESI): m/z: [M+H] calculated for C₂₁H₂₇F₃N₅O₃S: 486.2. found 486.1; ¹H NMR (400 MHz, METHANOL-d₄) δ ppm 6.90 (d, J=10.8 Hz, 2H), 6.78 (s, 1H), 5.44-5.34 (m, 1H), 4.61 (s, 2H), 4.53 (d, J=2.0 Hz, 2H), 4.05-3.97 (m, 2H), 3.59-3.49 (m, 1H), 3.46-3.37 (m, 2H), 2.39 (s, 3H), 2.01-1.80 (m, 4H), 1.51 (d, J=7.2 Hz, 3H).

The examples in the following Table 3 were synthesized in the manner similar to Example 47.

TABLE 3
Examples 48-52 and 187

| Example # | Structure | Mass found |
|---|---|---|
| Example 48. (See also Example 166.) | 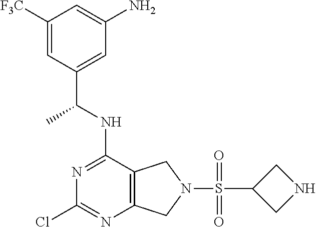 [SMILES: C[C@@H](Nc1nc(Cl)nc2c1CN(S(=O)(=O)C1CNC1)C2)c1cc(N)cc(C(F)(F)F)c1] | 477.4 |
| Example 49. | 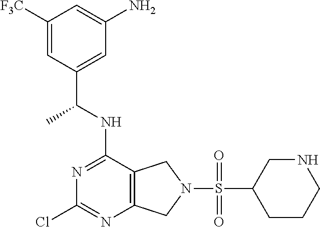 [SMILES: C[C@@H](Nc1nc(Cl)nc2c1CN(S(=O)(=O)C1CCCNC1)C2)c1cc(N)cc(C(F)(F)F)c1] | 505.4 |
| Example 50. | 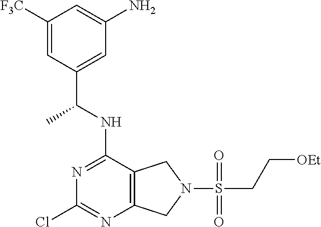 [SMILES: CCOCCS(=O)(=O)N1Cc2nc(Cl)nc(N[C@H](C)c3cc(N)cc(C(F)(F)F)c3)c2C1] | 494.4 |
| Example 51. | 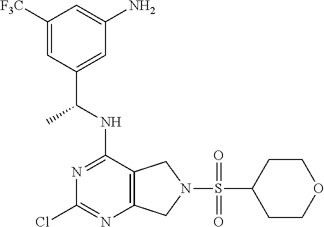 [SMILES: C[C@@H](Nc1nc(Cl)nc2c1CN(S(=O)(=O)C1CCOCC1)C2)c1cc(N)cc(C(F)(F)F)c1] | 506.4, 507.2 |
| Example 52. | 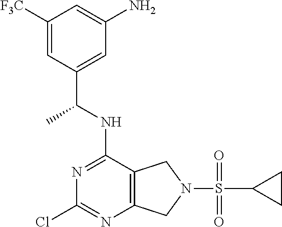 [SMILES: C[C@@H](Nc1nc(Cl)nc2c1CN(S(=O)(=O)C1CC1)C2)c1cc(N)cc(C(F)(F)F)c1] | 462.27 |
| Example 187. | 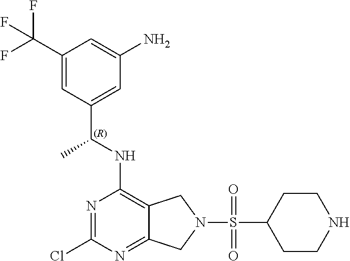 | 505.0 |

### Example 53. Synthesis of [2-chloro-4-[1-[5-[2-(hydroxymethyl)phenyl]-2-thienyl]ethylamino]-5,7-dihydropyrrolo[3,4-d]pyrimidin-6-yl]-morpholino-methan one

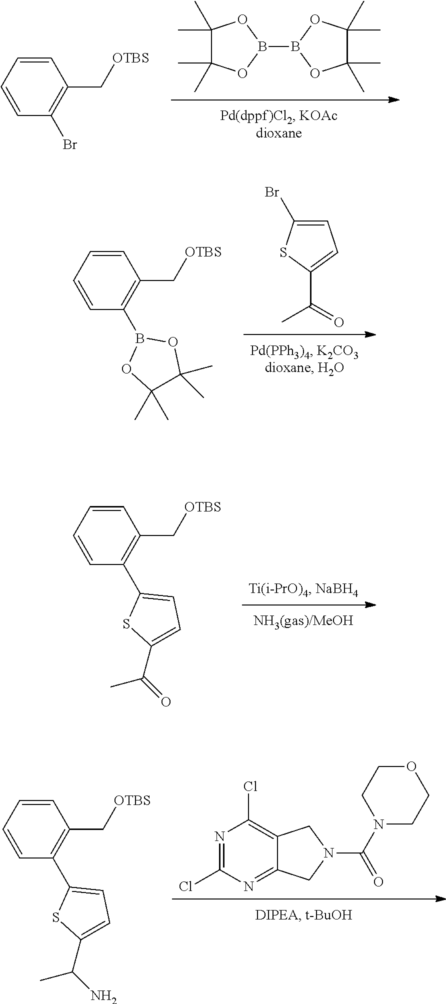

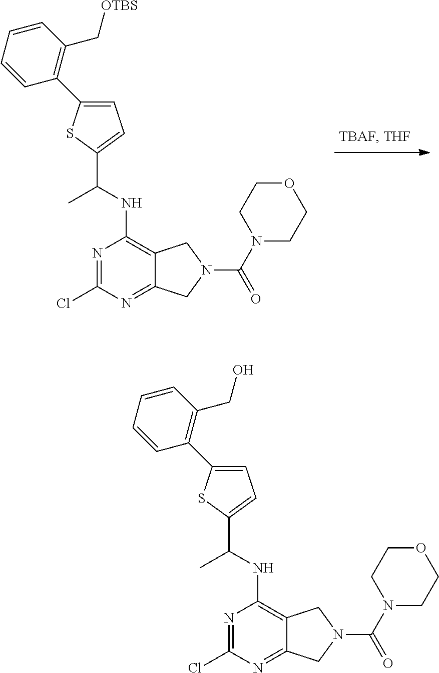 [SMILES: CC(Nc1nc(Cl)nc2c1CN(C(=O)N1CCOCC1)C2)c1ccc(-c2ccccc2CO[Si](C)(C)C(C)(C)C)s1 ; CC(Nc1nc(Cl)nc2c1CN(C(=O)N1CCOCC1)C2)c1ccc(-c2ccccc2CO)s1]

### Step 1.

To a mixture of (2-bromophenyl)methoxy-tert-butyl-dimethylsilane (1.0 g, 3.3 mmol) and bis(pinacolato)diboron (1.0 g, 4.0 mmol) in 1,4-dioxane (15 mL) under an atmosphere of N₂ was added KOAc (652 mg, 6.64 mmol) and Pd(dppf)Cl₂.CH₂Cl₂ (271 mg, 0.33 mmol). The mixture was heated to 100° C. and stirred for 10 h. H₂O (20 mL) was added and the mixture was extracted with EtOAc (20 mL×2). The combined organic layers were washed with brine (20 mL), dried over anhydrous Na₂SO₄, and filtered. The solvent was concentrated under reduced pressure and the residue was purified by column chromatography to give tert-butyl-dimethyl-[[2-(4,4,5,5-tetramethyl-1,3,2-dioxaborolan-2-yl)phenyl]methoxy]silane (1 g, 87% yield). ¹H NMR (400 MHz, CDCl₃) δ ppm 7.79-7.75 (m, 1H), 7.61 (d, J=7.6 Hz, 1H), 7.45 (dt, J=7.6 1.6 Hz, 1H), 7.26-7.21 (m, 1H), 5.03 (s, 2H), 1.34 (s, 12H), 0.96 (s, 9H), 0.10 (s, 6H).

### Step 2.

To a mixture of tert-butyl-dimethyl-[[2-(4,4,5,5-tetramethyl-1,3,2-dioxaborolan-2-yl)phenyl]methoxy]silane (1.0 g, 2.9 mmol) and 1-(5-bromo-2-thienyl)ethanone (589 mg, 2.9 mmol) in 1,4-dioxane (10 mL) and H₂O (2 mL) was added Pd(PPh₃)₄(332 mg, 0.29 mmol) and K₂CO₃ (1.59 g, 11.48 mmol). The mixture was heated to 100° C. and stirred for 10 h. H₂O (20 mL) was added and the mixture was extracted with EtOAc (20 mL×2). The combined organic layers were washed with brine (20 mL), dried over anhydrous Na₂SO₄, and filtered. The solvent was concentrated under reduced pressure and the residue was purified by column chromatography to give 1-[5-[2-[[tert-butyl(dimethyl)silyl]oxymethyl]phenyl]-2-thienyl]ethanone (0.85 g, 85% yield). ¹H NMR (400 MHz, CDCl₃) δ ppm 7.68 (d, J=4.0 Hz, 1H), 7.62 (d, J=7.6 Hz, 1H), 7.42 (d, J=7.6 Hz, 2H), 7.34 (s, 1H), 7.18 (d, J=4.0 Hz, 1H), 4.76 (s, 2H), 2.60 (s, 3H), 0.93 (s, 9H), 0.08 (s, 6H).

### Step 3.

To a mixture of 1-[5-[2-[[tert-butyl(dimethyl)silyl]oxymethyl]phenyl]-2-thienyl]ethanone (0.8 g, 2.3 mmol) in MeOH (10 mL) was added NH₃ (118 mg, 6.93 mmol) and Ti(i-PrO)₄ (1.36 mL, 4.62 mmol). The mixture was stirred rt for 10 h, then NaBH₄ (131 mg, 3.46 mmol) was added, and the mixture stirred at rt for 1 h. The mixture was poured into ice-H₂O (10 mL) and extracted with EtOAc (10 mL×3). The combined organic layers were washed with brine (20 mL), dried with anhydrous Na₂SO₄, and filtered. The solvent was concentrated under reduced pressure and the residue was purified by column chromatography to give 1-[5-[2-[[tert-butyl(dimethyl)silyl]oxymethyl]phenyl]-2-thienyl]ethanamine (400 mg, 50% yield). H NMR (400 MHz, DMSO-d₆) δ ppm 7.53 (d, J=7.2 Hz, 1H), 7.29-7.40 (m, 3H), 7.04 (d, J=3.6 Hz, 1H), 6.93 (d, J=3.6 Hz, 1H), 4.74 (s, 2H), 4.23 (q, J=6.4 Hz, 1H), 2.12 (br s, 2H), 1.37 (d, J=6.4 Hz, 3H), 0.88 (s, 9H), 0.05 (s, 6H).

### Step 4.

To a mixture of 1-[5-[2-[[tert-butyl(dimethyl)silyl]oxymethyl]phenyl]-2-thienyl]ethanamine (100 mg, 0.29 mmol) and (2,4-dichloro-5,7-dihydropyrrolo[3,4-d]pyrimidin-6-yl)-morpholino-methanone (87 mg, 0.29 mmol) in t-BuOH (3 mL) was added DIPEA (100 μL, 0.58 mmol). The mixture was heated to 100° C. in a crimped vial and stirred for 10 h, then diluted with H₂O (10 mL) and extracted with EtOAc (5 mL×2). The combined organic layers were washed with brine (10 mL), dried over anhydrous Na₂SO₄, and filtered. The solvent was concentrated under reduced pressure and the residue was purified by column chromatography to give [4-[1-[5-[2-[[tert-butyl(dimethyl)silyl]oxymethyl]phenyl]-2-thienyl]ethylamino]-2-chloro-5,7-dihydropyrrolo[3,4-d]pyrimidin-6-yl]-morpholino-methanone (150 mg, 85% yield). LCMS (ESI): m/z: [M+H] calculated for C₃₀H₄₁ClN₅O₃SSi: 614.2. found 614.2.

### Step 5.

To a mixture of [4-[1-[5-[2-[[tert-butyl(dimethyl)silyl]oxymethyl]phenyl]-2-thienyl]ethylamino]-2-chloro-5,7-dihy dropyrrolo[3,4-d]pyrimidin-6-yl]-morpholino-methanone (100 mg, 0.16 mmol) in THF (5 mL) was added 1M TBAF in THF (326 μL, 0.33 mmol). The mixture was stirred at rt for 1 h, then poured into ice-H₂O (5 mL) and extracted with EtOAc (5 mL×4). The combined organic layers were washed with brine (5 mL), dried with anhydrous Na₂SO₄, and filtered. The solvent was concentrated under reduced pressure and the residue was purified by prep-HPLC to give [2-chloro-4-[1-[5-[2-(hydroxymethyl)phenyl]-2-thienyl]ethylamino]-5,7-dihydropyrrolo[3,4-d]pyrimidin-6-yl]-morpholino-methanone (45 mg, 55% yield). LCMS (ESI): m/z: [M+H] calculated for C₂₄H₂₇ClN₅O₃S: 500.14. found 500.2; ¹H NMR (400 MHz, DMSO-d₆) δ ppm 8.28 (d, J=8.0 Hz, 1H), 7.57 (d, J=7.2 Hz, 1H), 7.39-7.33 (m, 2H), 7.32-7.27 (m, 1H), 7.12 (d, J=3.6 Hz, 1H), 7.06 (d, J=2.8 Hz, 1H), 5.59 (br t, J=7.2 Hz, 1H), 5.24 (t, J=5.6 Hz, 1H), 4.58-4.48 (m, 6H), 3.66-3.58 (m, 4H), 3.26-3.21 (m, 4H), 1.62 (d, J=6.8 Hz, 3H).

### Example 54. (See also Example 110.) Synthesis of N-[(1R)-1-[3-amino-5-(trifluoromethyl)phenyl]ethyl]-6-(tetrahydrofuran-3-ylmethyl)-5,7-dihydropyrrolo[3,4-d]pyrimidin-4-amine

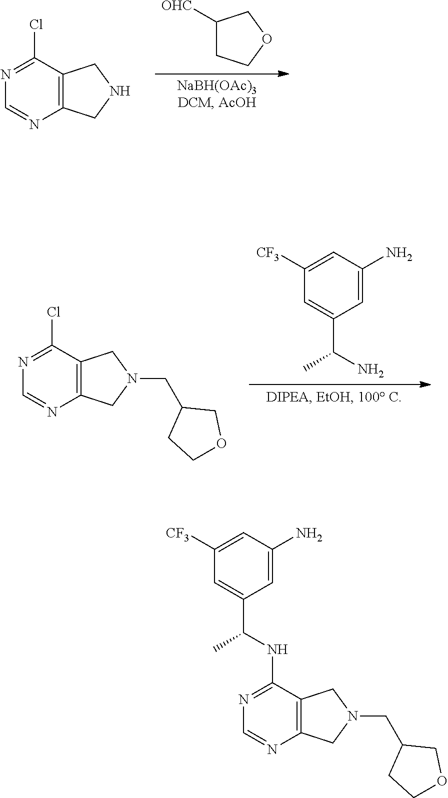

### Step 1.

To a mixture of 4-chloro-6,7-dihydro-5H-pyrrolo[3,4-d]pyrimidine TFA salt (450 mg, 1.67 mmol) and tetrahydrofuran-3-carbaldehyde (151 μL, 1.67 mmol) in DCM (3.5 mL) and AcOH (1.5 mL) was added NaBH(OAc)₃ (884 mg, 4.17 mmol). The mixture was stirred at rt for 1 h then concentrated under reduced pressure. The residue was diluted with H₂O (20 mL) and extracted with EtOAc (10 mL×3). The combined organic layers were washed with brine (20 mL), dried over Na₂SO₄, filtered and concentrated under reduced pressure to give a residue. The solvent was concentrated under reduced pressure and the residue was purified by column chromatography to give 4-chloro-6-(tetrahydrofuran-3-ylmethyl)-5,7-dihydropyrrolo[3,4-d]pyrimidine (120 mg). LCMS (ESI): m/z: [M+H] calculated for C₁₁H₁₅ClN₃O: 240.1. found 240.1.

### Step 2.

To a mixture of 3-[(1R)-1-aminoethyl]-5-(trifluoromethyl)aniline (205 mg, 1.0 mmol) and 4-chloro-6-(tetrahydrofuran-3-ylmethyl)-5,7-dihydropyrrolo[3,4-d]pyrimidine (120 mg, 0.50 mmol) in EtOH (2.5 mL) was added DIPEA (872 μL, 5.0 mmol). The mixture was heated to 100° C. and stirred for 2 h, then the solvent was concentrated under reduced pressure and the residue was purified by prep-HPLC to give N-[(1R)-1-[3-amino-5-(trifluoromethyl)phenyl]ethyl]-6-(tetrahydrofuran-3-ylmethyl)-5,7-dihydropyrrolo[3,4-d]pyrimidin-4-amine (10 mg, 5% yield). LCMS (ESI): m/z: [M+H] calculated for C₂₀H₂₅F₃N₅O: 408.2. found 408.1; ¹H NMR (400 MHz, METHANOL-d₄) δ ppm 8.27 (s, 1H), 6.89 (s, 2H), 6.78 (s, 1H), 5.32 (q, J=6.8 Hz, 1H), 3.97-3.83 (m, 6H), 3.77 (q, J=7.8 Hz, 1H), 3.55 (dd, J=8.6, 6.4 Hz, 1H), 2.84-2.73 (m, 2H), 2.53 (td, J=14.4, 7.2 Hz, 1H), 2.18-2.07 (m, 1H), 1.75-1.56 (m, 1H), 1.52 (d, J=6.8 Hz, 3H).

### Example 55. Synthesis of [4-[[(1R)-1-[3-amino-(trifluoromethyl)phenyl]ethyl]amino]-2-methoxy-5,7-dihydropyrrolo[3,4-d]pyrimidin-6-yl]-morpholino-methanone and [4-[[(1R)-1-[3-amino-5-(trifluoromethyl)phenyl]ethyl]amino]-2-hydroxy-5,7-dihydropyrrolo[3,4-d]pyrimidin-6-yl]-morpholino-methanone

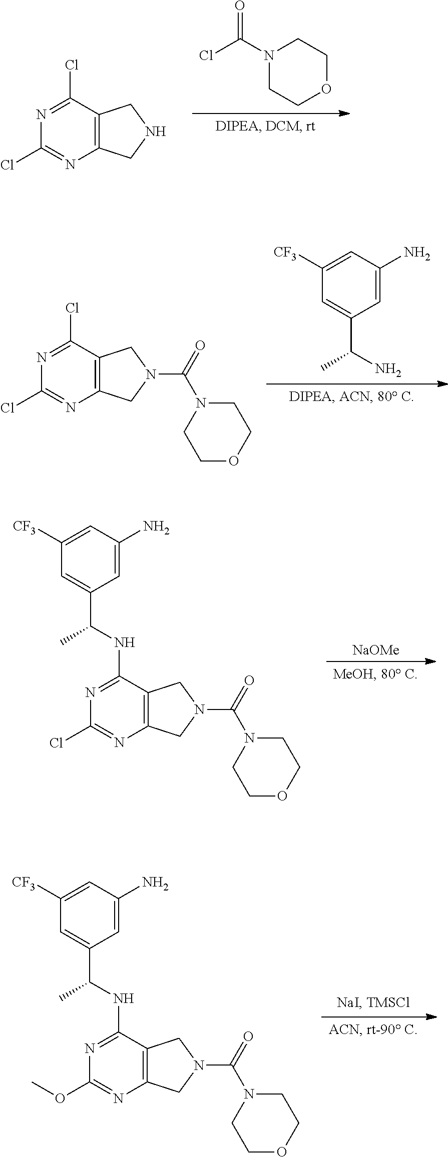

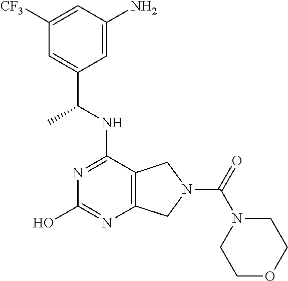 [SMILES: C[C@@H](Nc1nc(O)nc2c1CN(C(=O)N1CCOCC1)C2)c1cc(N)cc(C(F)(F)F)c1]

### Step 1.

To a mixture of 2,4-dichloro-6,7-dihydro-5H-pyrrolo[3,4-d]pyrimidine HCl salt (340 mg, 1.5 mmol) and morpholine-4-carbonyl chloride (175 μL, 1.5 mmol) in DCM (5 mL) was added DIPEA (1.3 mL, 7.5 mmol). The mixture was stirred at rt for 3 h, then the solvent was concentrated under reduced pressure and the residue was purified by column chromatography to give (2,4-dichloro-5,7-dihydropyrrolo[3,4-d]pyrimidin-6-yl)-morpholino-methanone (400 mg, 88% yield). LCMS (ESI): m/z: [M+H] calculated for C₁₁H₁₃Cl₂N₄O₂: 303.03. found: 302.8; ¹H NMR (400 MHz, CD₃CN) δ ppm 4.76 (s, 4H), 3.63-3.69 (m, 4H), 3.25-3.31 (m, 4H).

### Step 2.

To a mixture of (2,4-dichloro-5,7-dihydropyrrolo[3,4-d]pyrimidin-6-yl)-morpholino-methanone (400 mg, 1.32 mmol) and 3-[(1R)-1-aminoethyl]-5-(trifluoromethyl)aniline (269 mg, 1.32 mmol) in MeCN (10 mL) was added DIPEA (1.15 mL, 6.60 mmol). The mixture was heated to 80° C. and stirred for 2 h, then the solvent was concentrated under reduced pressure and the residue was purified by column chromatography to give [4-[[(1R)-1-[3-amino-5-(trifluoromethyl)phenyl]ethyl]amino]-2-chloro-5,7-dihydropyrrolo[3,4-d]pyrimidin-6-yl]-morpholino-methanone (350 mg, 56% yield). LCMS (ESI): m/z: [M+H] calculated for C₂₀H₂₃ClF₃N₆O₂: 471.14. found: 470.9; ¹H NMR (400 MHz, CDCl₃) δ ppm 6.96 (s, 1H), 6.82 (s, 2H), 5.35 (br s, 1H), 4.85 (d, J=14.8 Hz, 1H), 4.55-4.62 (m, 4H), 3.92 (s, 2H), 3.70-3.75 (m, 4H), 3.32-3.37 (m, 4H), 1.59 (d, J=6.8 Hz, 3H).

### Step 3.

To a mixture of [4-[[(1R)-1-[3-amino-5-(trifluoromethyl)phenyl]ethyl]amino]-2-chloro-5,7-dihydropyrrolo[3,4-d]pyrimidin-6-yl]-morpholino-methanone (110 mg, 0.23 mmol) in MeOH (1 mL) was added sodium methoxide, 30% purity (84 mg, 0.47 mmol). The mixture was heated to 80° C. in a crimped vial and stirred for 2 h, then saturated NH₄Cl (2 mL) was added and the mixture extracted with EtOAc (2 mL×3). The combined organic layers were washed with brine (2 mL), dried over anhydrous Na₂SO₄, and filtered. The solvent was concentrated under reduced pressure and the residue was purified by prep-HPLC to give [4-[[(1R)-1-[3-amino-(trifluoromethyl)phenyl]ethyl]amino]-2-methoxy-5,7-dihydropyrrolo[3,4-d]pyrimidin-6-yl]-morpholino-methanone (42 mg, 39% yield). LCMS (ESI): m/z: [M+H] calculated for C₂₁H₂₆F₃N₆O₃: 467.20. found: 467.0; ¹H NMR (400 MHz, METHANOL-d₄) δ ppm 6.91 (d, J=8.8 Hz, 2H), 6.79 (s, 1H), 5.25 (br d, J=6.4 Hz, 1H), 4.59 (s, 2H), 4.47-4.55 (m, 2H), 3.78 (s, 3H), 3.69-3.75 (m, 4H), 3.35 (d, J=4.0 Hz, 4H), 1.54 (d, J=7.1 Hz, 3H).

### Step 4.

To a mixture of [4-[[(1R)-1-[3-amino-5-(trifluoromethyl)phenyl]ethyl]amino]-2-methoxy-5,7-dihydropyrrolo[3,4-d]pyrimidin-6-yl]-morpholino-methanone (200 mg, 0.43 mmol) and NaI (122 mg, 0.82 mmol) in MeCN (5 mL) was added TMSCl (109 μL, 0.86 mmol). The mixture was heated to 90° C. in a crimped vial and stirred for 12 h. The mixture was filtered, the solvent was concentrated under reduced pressure and the residue was purified by prep-HPLC to give [4-[[(1R)-1-[3-amino-5-(trifluoromethyl)phenyl]ethyl]amino]-2-hydroxy-5,7-dihydropyrrolo[3,4-d]pyrimidin-6-yl]-morpholino-methanone (83 mg, 43% yield). LCMS (ESI): m/z: [M+H] calculated for C₂₀H₂₄F₃N₆O₃: 453.18. found: 453.1; H NMR (400 MHz, CDCl₃) δ ppm 6.95 (m, 2H), 6.79 (s, 1H), 5.43 (m, 1H) 4.56 (s, 2H), 4.50 (s, 2H), 3.71 (m, 4H), 3.46 (m, 4H), 1.51 (d, J=6.8 Hz, 3H).

### Example 56. Synthesis of N-[(1R)-1-[3-amino-5-(trifluoromethyl)phenyl]ethyl]-2-methyl-6-(tetrahydrofuran-3-ylmethyl)-7,8-dihydro-5H-pyrido[4,3-d]pyrimidin-4-amine

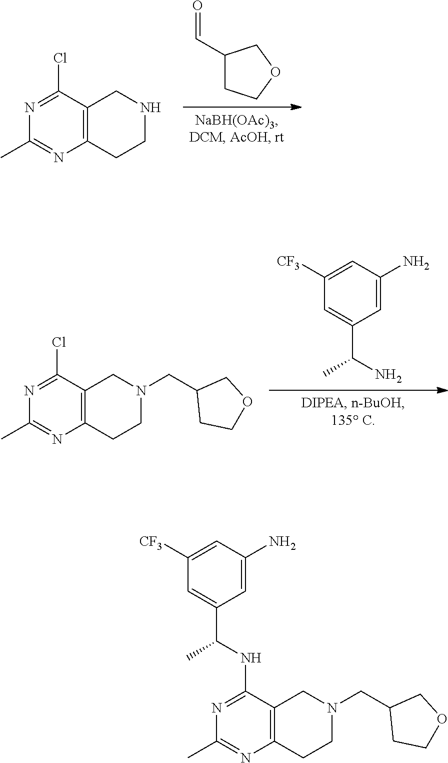

### Step 1.

To a mixture of 4-chloro-2-methyl-5,6,7,8-tetrahydropyrido[4,3-d]pyrimidine TFA salt (0.53 g, 1.78 mmol) and tetrahydrofuran-3-carbaldehyde (482 μL, 5.34 mmol) in DCM (7 mL) was added AcOH (3 mL) and NaBH(OAc)₃ (1.89 g, 8.9 mmol). The mixture was stirred at rt for 12 h and the solvent was concentrated under reduced pressure, then diluted with H₂O (20 mL) and extracted with EtOAc (10 mL×5). The combined organic layers were washed with brine (30 mL), dried over Na₂SO₄, and filtered. The solvent was concentrated under reduced pressure and the residue was purified by column chromatography to give 4-chloro-2-methyl-6-(tetrahydrofuran-3-ylmethyl)-7,8-dihydro-5H-pyrido[4,3-d]pyrimidine (0.23 g, 48% yield). ¹H NMR (400 MHz, METHANOL-d₄) δ ppm 3.94-3.85 (m, 2H), 3.84-3.70 (m, 4H), 3.60-3.53 (m, 1H), 3.11-2.96 (m, 4H), 2.78 (d, J=8.0 Hz, 2H), 2.61 (s, 3H), 2.19-2.08 (m, 1H), 1.74-1.68 (m, 1H).

### Step 2.

To a mixture of 4-chloro-2-methyl-6-(tetrahydrofuran-3-ylmethyl)-7,8-dihydro-5H-pyrido[4,3-d]pyrimidine (0.13 g, 0.49 mmol) in n-BuOH (2 mL) was added 3-[(1R)-1-aminoethyl]-5-(trifluoromethyl)aniline (198 mg, 0.97 mmol) and DIPEA (846 μL, 4.86 mmol). The mixture was heated to 135° C. in a crimped vial and stirred for 12 h, then the solvent was concentrated under reduced pressure and the residue was purified by prep-HPLC (×2) to give N-[(1R)-1-[3-amino-5-(trifluoromethyl)phenyl]ethyl]-2-methyl-6-(tetrahydrofuran-3-ylmethyl)-7,8-dihydro-5H-pyrido[4,3-d]pyrimidin-4-amine (35 mg, 17% yield). LCMS (ESI): m/z: [M+H] calculated for C₂₂H₂₉F₃N₅O: 436.2. found 436.1; H NMR (400 MHz, METHANOL-d₄) δ ppm 6.92 (s, 1H), 6.90 (s, 1H), 6.78 (s, 1H), 5.52-5.38 (m, 1H), 3.94-3.84 (m, 2H), 3.79-3.74 (m, 1H), 3.58-3.52 (m, 1H), 3.40 (d, J=8.0 Hz, 2H), 2.86-2.76 (m, 2H), 2.75-2.65 (m, 3H), 2.63-2.58 (m, 2H), 2.34 (s, 3H), 2.14-2.07 (m, 1H), 1.74-1.62 (m, 1H), 1.53 (d, J=4.0 Hz, 3H).

### Example 57. Synthesis of 4-[4-[[(1R)-1-[3-amino-5-(trifluoromethyl)phenyl]ethyl]amino]-2-chloro-5,7-dihydropyrrolo[3,4-d]pyrimidin-6-yl]-N,N-dimethyl-cyclohexanecarboxamide

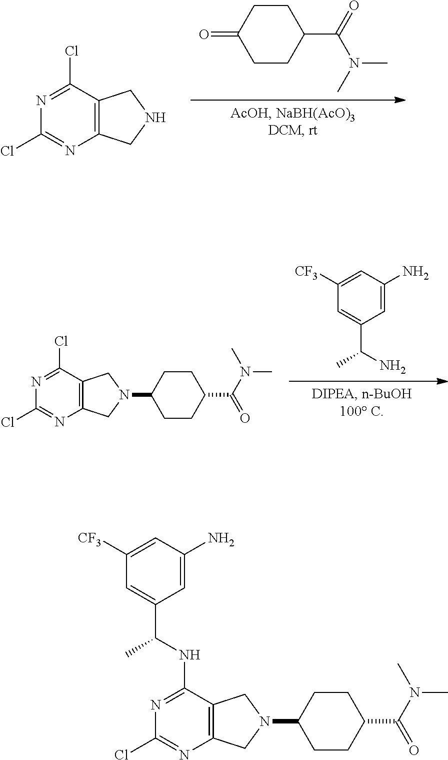

### Step 1.

To a mixture of 2,4-dichloro-6,7-dihydro-5H-pyrrolo[3,4-d]pyrimidine TFA salt (1.68 g, 5.53 mmol), N,N-dimethyl-4-oxo-cyclohexanecarboxamide (935 mg, 5.53 mmol) in DCM (11.2 mL) and AcOH (4.8 mL) was added NaBH(OAc)₃ (4.10 g, 19.3 mmol). The mixture was stirred at rt for 3 h, then the solvent was concentrated under reduced pressure and the residue was purified by column chromatography to give 4-(2,4-dichloro-5,7-dihydropyrrolo[3,4-d]pyrimidin-6-yl)-N,N-dimethyl-cyclohexanecarboxamide (150 mg, 8% yield). LCMS (ESI): m/z: [M+H] calculated for C₁₅H₂₁Cl₂N₄O: 343.10. found 343.2.

### Step 2.

To a mixture of 3-[(1R)-1-aminoethyl]-5-(trifluoromethyl)aniline (93 mg, 0.46 mmol) and 4-(2,4-dichloro-5,7-dihydropyrrolo[3,4-d]pyrimidin-6-yl)-N,N-dimethyl-cyclohexanecarboxamide (120 mg, 0.35 mmol) in n-BuOH (1 mL) was added DIPEA (609 μL, 3.5 mmol). The mixture was heated to 100° C. and stirred for 5 h, then the solvent was concentrated under reduced pressure and the residue was purified by prep-HPLC to give 4-[4-[[(1R)-1-[3-amino-5-(trifluoromethyl)phenyl]ethyl]amino]-2-chloro-5,7-dihydropyrrolo[3,4-d]pyrimidin-6-yl]-N,N-dimethyl-cyclohexanecarboxamide (20 mg, 11% yield). LCMS (ESI): m/z: [M+H] calculated for C₂₄H₃₁ClF₃N₆O: 511.21. found 511.4; ¹H NMR (400 MHz, CDCl₃) δ ppm 6.95 (s, 1H), 6.81 (s, 2H), 5.31 (m, 1H), 4.73-4.85 (m, 1H), 3.73-3.95 (m, 5H), 3.06 (s, 3H), 2.95 (s, 3H), 2.42-2.56 (m, 2H), 2.09 (br d, J=10.8 Hz, 2H), 1.84 (m, 2H), 1.60-1.71 (m, 2H), 1.56 (d, J=6.8 Hz, 3H), 1.27-1.30 (m, 2H).

### Example 58. Synthesis of [4-[[(1R)-1-[3-amino-5-(trifluoromethyl)phenyl]ethyl]amino]-2-chloro-5,7-dihydropyrrolo[3,4-d]pyrimidin-6-yl]-(1-methyl-4-piperidyl)methanone

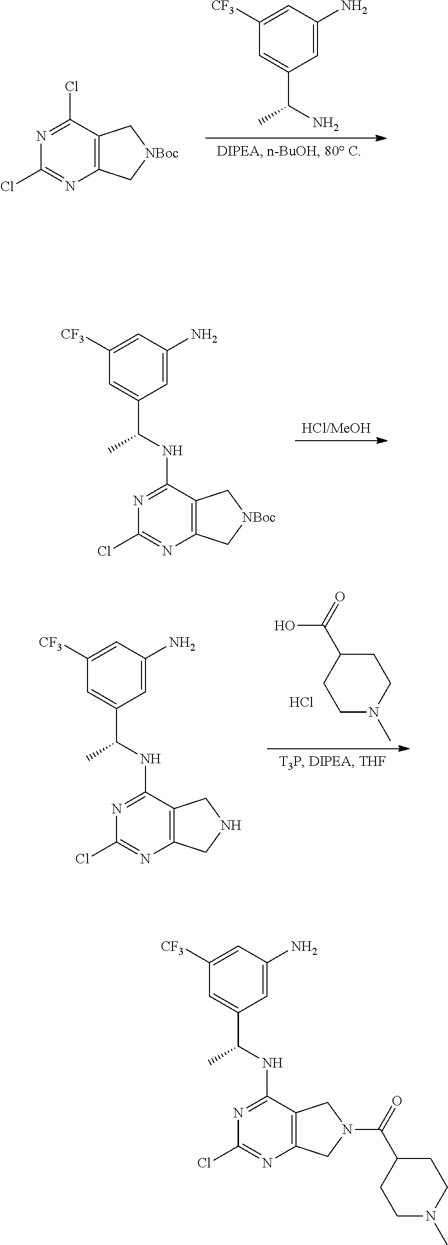

### Step 1.

To a mixture of 3-[(1R)-1-aminoethyl]-5-(trifluoromethyl)aniline (265 mg, 1.3 mmol) in n-BuOH (15 mL) was added tert-butyl 2,4-dichloro-5,7-dihydropyrrolo[3,4-d]pyrimidine-6-carboxylate (301 mg, 1.0 mmol) and DIPEA (678 μL, 3.9 mmol). The mixture was heated to 80° C. and stirred for 10 h, then the solvent was concentrated under reduced pressure and the residue was purified by column chromatography to give tert-butyl 4-[[(1R)-1-[3-amino-5-(trifluoromethyl)phenyl]ethyl]amino]-2-chloro-5,7-dihydropyrrolo[3,4-d]pyrimidine-6-carboxylate (385 mg, 65% yield). ¹H NMR (400 MHz, METHANOL-d₄) δ ppm 6.91 (m, 2H), 6.81 (s, 1H), 5.33-5.30 (m, 1H), 4.51-4.43 (m, 4H), 1.54-1.48 (m, 12H).

### Step 2.

A mixture of tert-butyl 4-[[(1R)-1-[3-amino-5-(trifluoromethyl)phenyl]ethyl]amino]-2-chloro-5,7-dihydropyrrolo[3,4-d]pyrimidine-6-carboxylate (380 mg, 0.83 mmol) in 4M HCl in MeOH (15 mL, 60 mmol) was stirred at 15° C. for 1.5 h. The solvent was concentrated under reduced pressure to give N-[(1R)-1-[3-amino-5-(trifluoromethyl)phenyl]ethyl]-2-chloro-6,7-dihydro-5H-pyrrolo[3,4-d]pyrimidin-4-amine HCl salt (350 mg). ¹H NMR (400 MHz, METHANOL-d₄) δ ppm 7.88 (s, 1H), 7.82 (s, 1H), 7.61 (s, 1H), 5.44 (q, J=7.2 Hz, 1H), 4.67-4.53 (m, 2H), 4.42 (s, 2H), 1.64 (d, J=7.2 Hz, 3H).

### Step 3.

To a mixture of N-[(1R)-1-[3-amino-5-(trifluoromethyl)phenyl]ethyl]-2-chloro-6,7-dihydro-SH-pyrrolo[3,4-d]pyrimidin-4-amine HCl salt (350 mg, 0.89 mmol) in THF (5 mL) at 15° C. was added 1-methylpiperidine-4-carboxylic acid (127 mg, 0.89 mmol), DIPEA (619 □L, 3.55 mmol) and T3P (396 μL, 1.33 mmol). The mixture was stirred at 15° C. for 1.5 h, then the solvent was concentrated under reduced pressure and the residue was purified by prep-HPLC to give [4-[[(1R)-1-[3-amino-5-(trifluoromethyl)phenyl]ethyl]amino]-2-chloro-5,7-dihydropyrrolo[3,4-d]pyrimidin-6-yl]-(1-methyl-4-piperidyl)methanone (85 mg, 20% yield). LCMS (ESI): m/z: [M+H] calculated for C22H₂₇ClF₃N₆O: 483.18. found 483.3; ¹H NMR (400 MHz, METHANOL-d₄) δ ppm 6.93-6.90 (m, 2H), 6.81 (d, J=1.6 Hz, 1H), 5.34 (d, J=7.2 Hz, 1H), 4.76-4.34 (m, 2H), 4.52 (d, J=7.2 Hz, 2H), 3.00-2.94 (m, 2H), 2.59-2.55 (m, 1H), 2.29 (s, 3H), 2.12-2.10 (m, 2H), 1.85-1.83 (m, 4H), 1.54 (dd, J=7.2, 5.2 Hz, 3H).

The examples in the following Table 4 were synthesized in the manner similar to Example 58.

TABLE 4
Examples 59-108 and 188-347

| Example # | Structure | mass found |
|---|---|---|
| Example 59. | 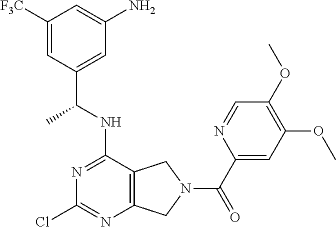 [SMILES: COc1cnc(C(=O)N2Cc3nc(Cl)nc(N[C@H](C)c4cc(N)cc(C(F)(F)F)c4)c3C2)cc1OC] | 523.4 |
| Example 60. | 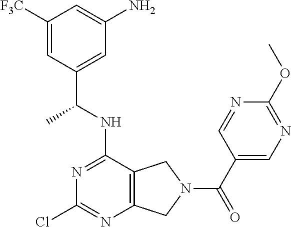 [SMILES: COc1ncc(C(=O)N2Cc3nc(Cl)nc(N[C@H](C)c4cc(N)cc(C(F)(F)F)c4)c3C2)cn1] | 494.5 |
| Example 61. | 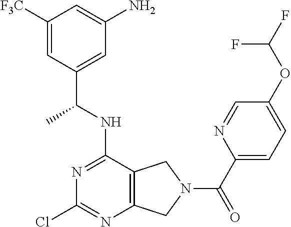 [SMILES: C[C@@H](Nc1nc(Cl)nc2c1CN(C(=O)c1ccc(OC(F)F)cn1)C2)c1cc(N)cc(C(F)(F)F)c1] | 529.4 |
| Example 62. | 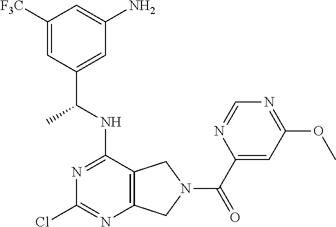 [SMILES: COc1cc(C(=O)N2Cc3nc(Cl)nc(N[C@H](C)c4cc(N)cc(C(F)(F)F)c4)c3C2)ncn1] | 494.4 |
| Example 63. | 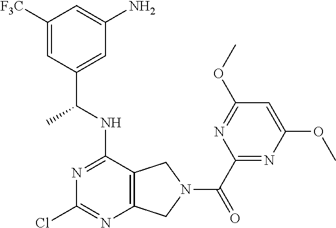 [SMILES: COc1cc(OC)nc(C(=O)N2Cc3nc(Cl)nc(N[C@H](C)c4cc(N)cc(C(F)(F)F)c4)c3C2)n1] | 524.5 |
| Example 64. | 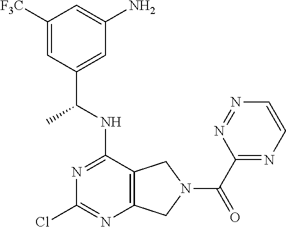 [SMILES: C[C@@H](Nc1nc(Cl)nc2c1CN(C(=O)c1nccnn1)C2)c1cc(N)cc(C(F)(F)F)c1] | 465.4 |
| Example 65. | 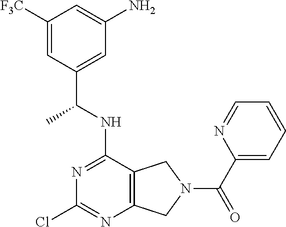 [SMILES: C[C@@H](Nc1nc(Cl)nc2c1CN(C(=O)c1ccccn1)C2)c1cc(N)cc(C(F)(F)F)c1] | 463.5 |
| Example 66. | 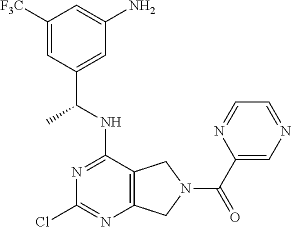 [SMILES: C[C@@H](Nc1nc(Cl)nc2c1CN(C(=O)c1cnccn1)C2)c1cc(N)cc(C(F)(F)F)c1] | 464.4 |
| Example 67. | 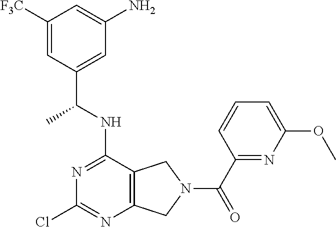 [SMILES: COc1cccc(C(=O)N2Cc3nc(Cl)nc(N[C@H](C)c4cc(N)cc(C(F)(F)F)c4)c3C2)n1] | 493.5 |
| Example 68. | 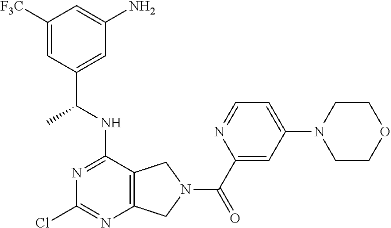 [SMILES: C[C@@H](Nc1nc(Cl)nc2c1CN(C(=O)c1cc(N3CCOCC3)ccn1)C2)c1cc(N)cc(C(F)(F)F)c1] | 548.5 |
| Example 69. | 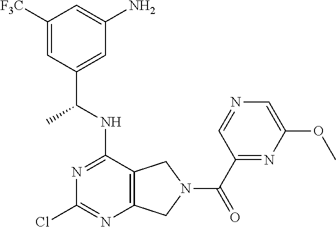 [SMILES: COc1cncc(C(=O)N2Cc3nc(Cl)nc(N[C@H](C)c4cc(N)cc(C(F)(F)F)c4)c3C2)n1] | 494.5 |
| Example 70. | 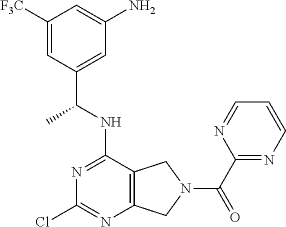 [SMILES: C[C@@H](Nc1nc(Cl)nc2c1CN(C(=O)c1ncccn1)C2)c1cc(N)cc(C(F)(F)F)c1] | 464.4 |
| Example 71. | 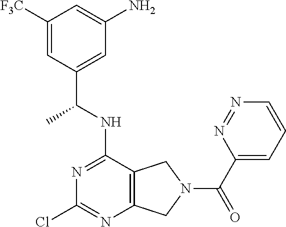 [SMILES: C[C@@H](Nc1nc(Cl)nc2c1CN(C(=O)c1cccnn1)C2)c1cc(N)cc(C(F)(F)F)c1] | 464.4 |
| Example 72. | 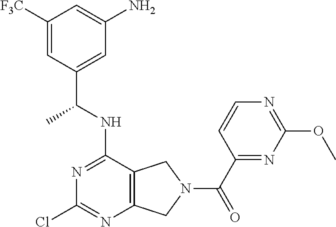 [SMILES: COc1nccc(C(=O)N2Cc3nc(Cl)nc(N[C@H](C)c4cc(N)cc(C(F)(F)F)c4)c3C2)n1] | 494.5 |
| Example 73. | 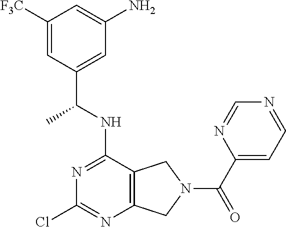 [SMILES: C[C@@H](Nc1nc(Cl)nc2c1CN(C(=O)c1ccncn1)C2)c1cc(N)cc(C(F)(F)F)c1] | 464.4 |
| Example 74. | 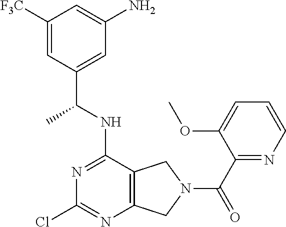 [SMILES: COc1cccnc1C(=O)N1Cc2nc(Cl)nc(N[C@H](C)c3cc(N)cc(C(F)(F)F)c3)c2C1] | 493.5 |
| Example 75. | 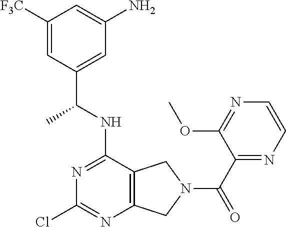 [SMILES: COc1nccnc1C(=O)N1Cc2nc(Cl)nc(N[C@H](C)c3cc(N)cc(C(F)(F)F)c3)c2C1] | 494.5 |
| Example 76. | 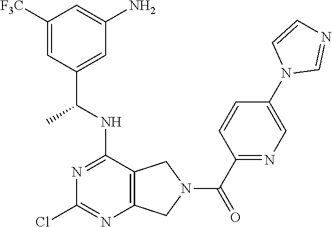 [SMILES: C[C@@H](Nc1nc(Cl)nc2c1CN(C(=O)c1ccc(-n3ccnc3)cn1)C2)c1cc(N)cc(C(F)(F)F)c1] | 529.5 |
| Example 77. | 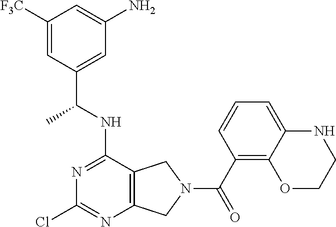 [SMILES: C[C@@H](Nc1nc(Cl)nc2c1CN(C(=O)c1cccc3c1OCCN3)C2)c1cc(N)cc(C(F)(F)F)c1] | 519.5 |
| Example 78. | 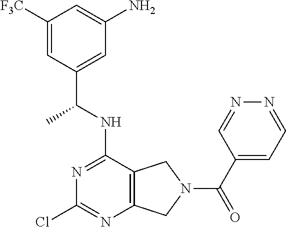 [SMILES: C[C@@H](Nc1nc(Cl)nc2c1CN(C(=O)c1ccnnc1)C2)c1cc(N)cc(C(F)(F)F)c1] | 464.4 |
| Example 79. | 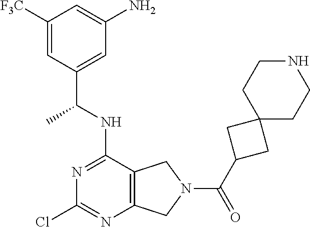 [SMILES: C[C@@H](Nc1nc(Cl)nc2c1CN(C(=O)C1CC3(CCNCC3)C1)C2)c1cc(N)cc(C(F)(F)F)c1] | 509.5 |
| Example 80. | 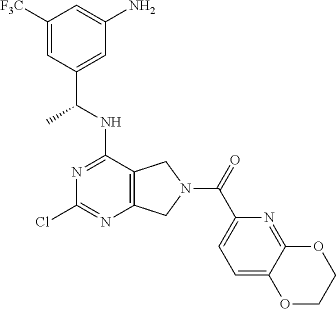 [SMILES: C[C@@H](Nc1nc(Cl)nc2c1CN(C(=O)c1ccc3c(n1)OCCO3)C2)c1cc(N)cc(C(F)(F)F)c1] | 521.4 |
| Example 81. | 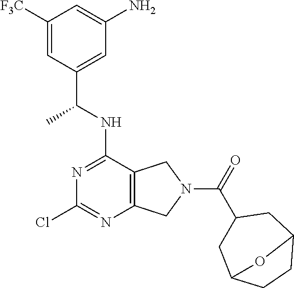 [SMILES: C[C@@H](Nc1nc(Cl)nc2c1CN(C(=O)C1CC3CCC(C1)O3)C2)c1cc(N)cc(C(F)(F)F)c1] | 496.5 |
| Example 82. | 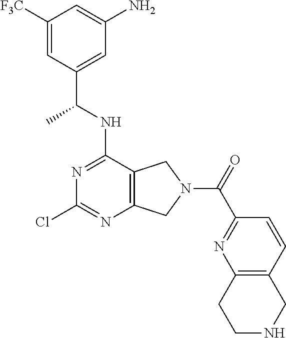 [SMILES: C[C@@H](Nc1nc(Cl)nc2c1CN(C(=O)c1ccc3c(n1)CCNC3)C2)c1cc(N)cc(C(F)(F)F)c1] | 518.5 |
| Example 83. | 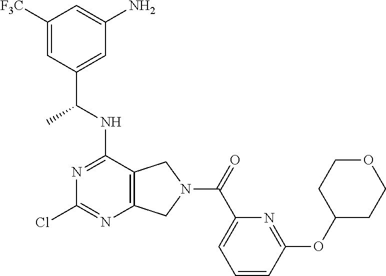 [SMILES: C[C@@H](Nc1nc(Cl)nc2c1CN(C(=O)c1cccc(OC3CCOCC3)n1)C2)c1cc(N)cc(C(F)(F)F)c1] | 563.4 |
| Example 84. | 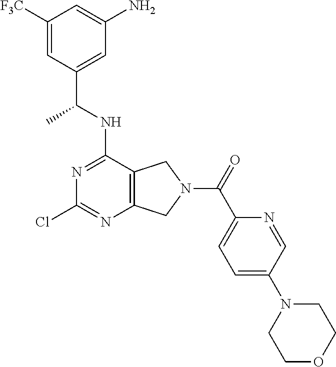 [SMILES: C[C@@H](Nc1nc(Cl)nc2c1CN(C(=O)c1ccc(N3CCOCC3)cn1)C2)c1cc(N)cc(C(F)(F)F)c1] | 548.5 |
| Example 85. | 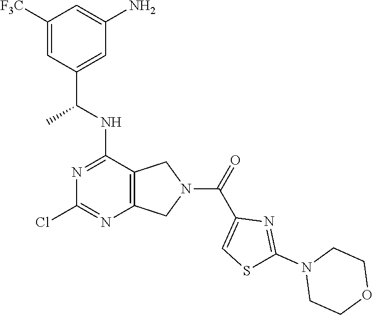 [SMILES: C[C@@H](Nc1nc(Cl)nc2c1CN(C(=O)c1csc(N3CCOCC3)n1)C2)c1cc(N)cc(C(F)(F)F)c1] | 554.4 |
| Example 86. | 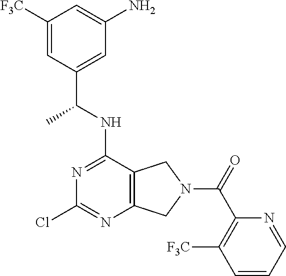 [SMILES: C[C@@H](Nc1nc(Cl)nc2c1CN(C(=O)c1ncccc1C(F)(F)F)C2)c1cc(N)cc(C(F)(F)F)c1] | 531.4 |
| Example 87. | 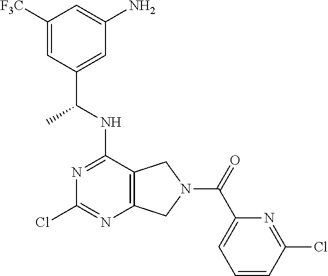 [SMILES: C[C@@H](Nc1nc(Cl)nc2c1CN(C(=O)c1cccc(Cl)n1)C2)c1cc(N)cc(C(F)(F)F)c1] | 497.4 |
| Example 88. | 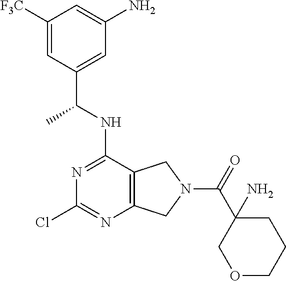 [SMILES: C[C@@H](Nc1nc(Cl)nc2c1CN(C(=O)C1(N)CCCOC1)C2)c1cc(N)cc(C(F)(F)F)c1] | 485.5 |
| Example 89. | 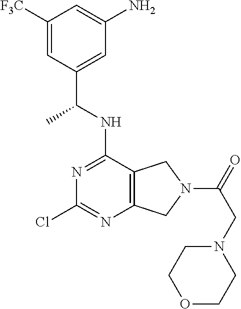 [SMILES: C[C@@H](Nc1nc(Cl)nc2c1CN(C(=O)CN1CCOCC1)C2)c1cc(N)cc(C(F)(F)F)c1] | 485.4 |
| Example 90. | 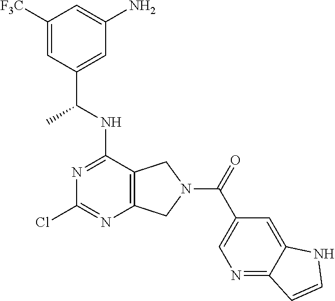 [SMILES: C[C@@H](Nc1nc(Cl)nc2c1CN(C(=O)c1cnc3cc[nH]c3c1)C2)c1cc(N)cc(C(F)(F)F)c1] | 502.4 |
| Example 91. | 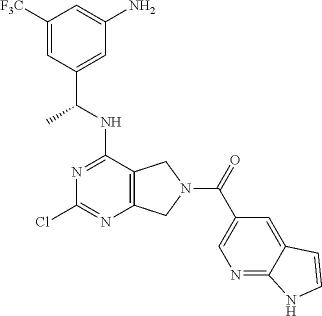 [SMILES: C[C@@H](Nc1nc(Cl)nc2c1CN(C(=O)c1cnc3[nH]ccc3c1)C2)c1cc(N)cc(C(F)(F)F)c1] | 502.4 |
| Example 92. | 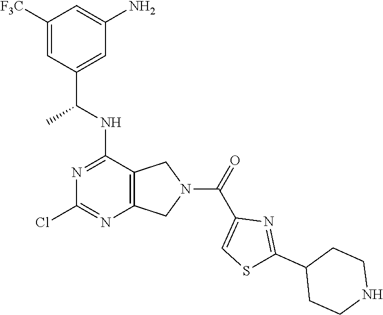 [SMILES: C[C@@H](Nc1nc(Cl)nc2c1CN(C(=O)c1csc(C3CCNCC3)n1)C2)c1cc(N)cc(C(F)(F)F)c1] | 552.5 |
| Example 93. | 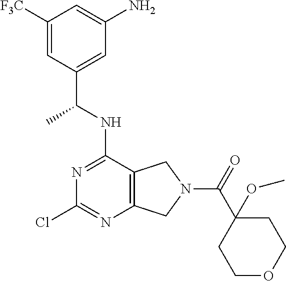 [SMILES: COC1(C(=O)N2Cc3nc(Cl)nc(N[C@H](C)c4cc(N)cc(C(F)(F)F)c4)c3C2)CCOCC1] | 500.5, 501.2, 500.1, 500 |
| Example 94. | 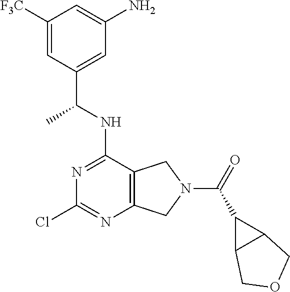 | 468.5 |
| Example 95. | 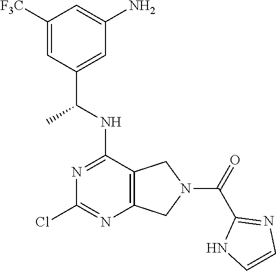 [SMILES: C[C@@H](Nc1nc(Cl)nc2c1CN(C(=O)c1ncc[nH]1)C2)c1cc(N)cc(C(F)(F)F)c1] | 452.2 |
| Example 96. | 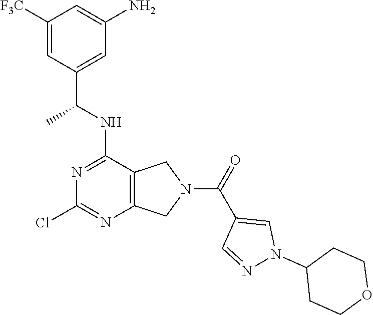 [SMILES: C[C@@H](Nc1nc(Cl)nc2c1CN(C(=O)c1cnn(C3CCOCC3)c1)C2)c1cc(N)cc(C(F)(F)F)c1] | 536.5 |
| Example 97. | 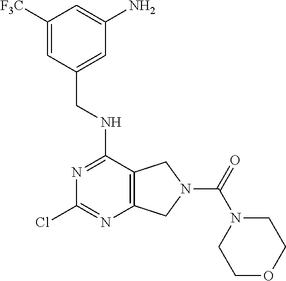 [SMILES: Nc1cc(CNc2nc(Cl)nc3c2CN(C(=O)N2CCOCC2)C3)cc(C(F)(F)F)c1] | 470.4 |
| Example 98. | 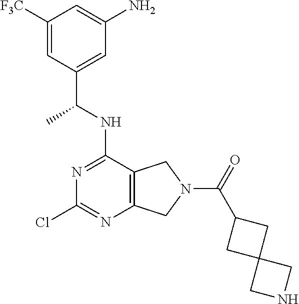 [SMILES: C[C@@H](Nc1nc(Cl)nc2c1CN(C(=O)C1CC3(CNC3)C1)C2)c1cc(N)cc(C(F)(F)F)c1] | 481.5 |
| Example 99. | 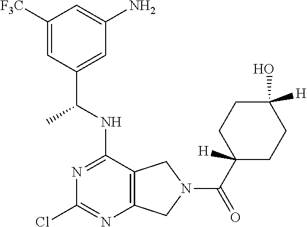 [SMILES: C[C@@H](Nc1nc(Cl)nc2c1CN(C(=O)[C@H]1CC[C@@H](O)CC1)C2)c1cc(N)cc(C(F)(F)F)c1] | 484.4 |
| Example 100. | 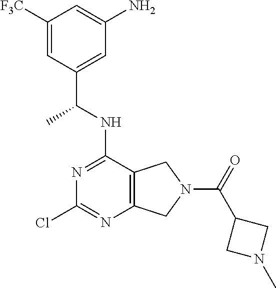 [SMILES: C[C@@H](Nc1nc(Cl)nc2c1CN(C(=O)C1CN(C)C1)C2)c1cc(N)cc(C(F)(F)F)c1] | 455.4 |
| Example 101. | 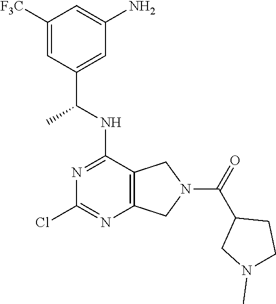 [SMILES: C[C@@H](Nc1nc(Cl)nc2c1CN(C(=O)C1CCN(C)C1)C2)c1cc(N)cc(C(F)(F)F)c1] | 469.5 |
| Example 102. | 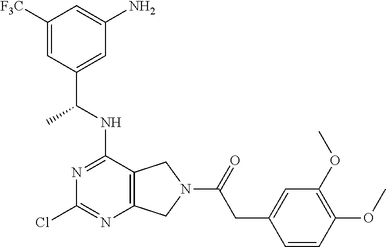 [SMILES: COc1ccc(CC(=O)N2Cc3nc(Cl)nc(N[C@H](C)c4cc(N)cc(C(F)(F)F)c4)c3C2)cc1OC] | 536.5 |
| Example 103. | 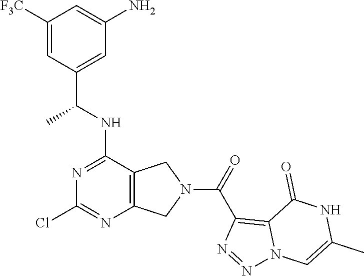 [SMILES: Cc1cn2nnc(C(=O)N3Cc4nc(Cl)nc(N[C@H](C)c5cc(N)cc(C(F)(F)F)c5)c4C3)c2c(=O)[nH]1] | 534.5 |
| Example 104. | 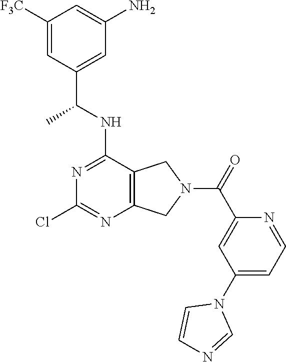 [SMILES: C[C@@H](Nc1nc(Cl)nc2c1CN(C(=O)c1cc(-n3ccnc3)ccn1)C2)c1cc(N)cc(C(F)(F)F)c1] | 529.39 |
| Example 105. | 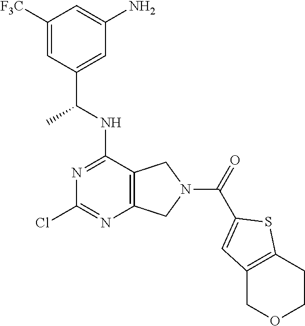 [SMILES: C[C@@H](Nc1nc(Cl)nc2c1CN(C(=O)c1cc3c(s1)CCOC3)C2)c1cc(N)cc(C(F)(F)F)c1] | 524.4 |
| Example 106. | 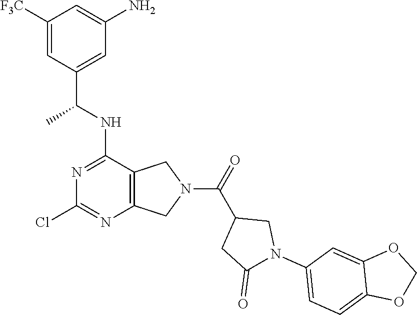 [SMILES: C[C@@H](Nc1nc(Cl)nc2c1CN(C(=O)C1CC(=O)N(c3ccc4c(c3)OCO4)C1)C2)c1cc(N)cc(C(F)(F)F)c1] | 589.5 |
| Example 107. | 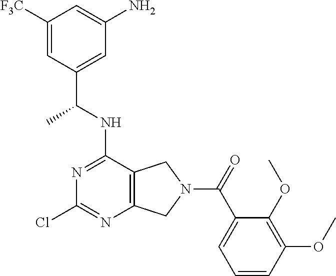 [SMILES: COc1cccc(C(=O)N2Cc3nc(Cl)nc(N[C@H](C)c4cc(N)cc(C(F)(F)F)c4)c3C2)c1OC] | 522.4 |
| Example 108. | 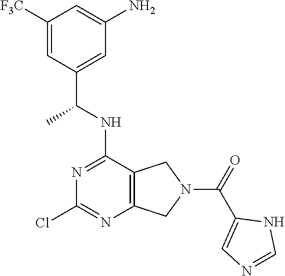 [SMILES: C[C@@H](Nc1nc(Cl)nc2c1CN(C(=O)c1cnc[nH]1)C2)c1cc(N)cc(C(F)(F)F)c1] | 452.4 |
| Example 188. | 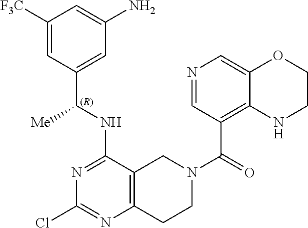 | 483.2 |
| Example 189. | 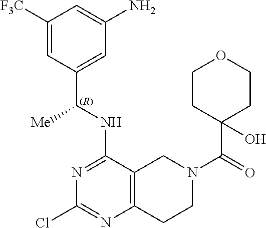 | 500.5 |
| Example 190. | 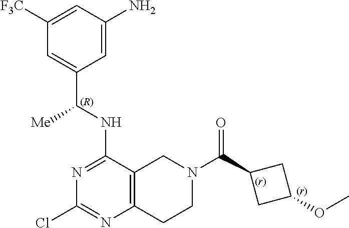 | 484.5 |
| Example 191. | 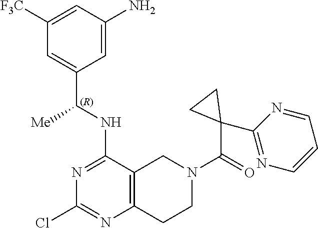 | 518.6 |
| Example 192. | 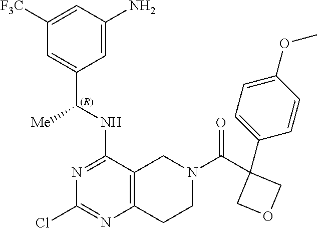 | 562.6 |
| Example 193. | 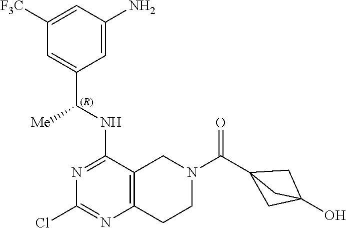 | 482.5 |
| Example 194. | 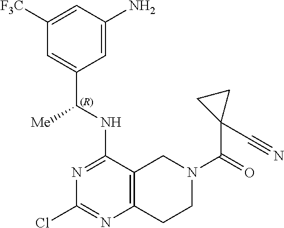 | 465.5 |
| Example 195. | 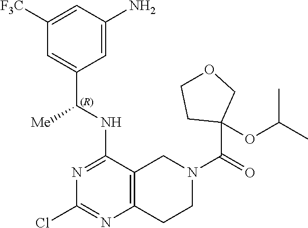 | 528.6 |
| Example 196. | 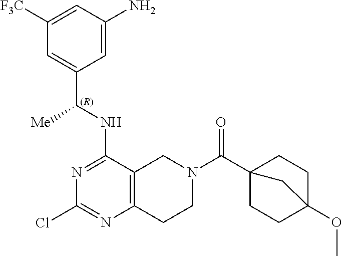 | 524.6 |
| Example 197. | 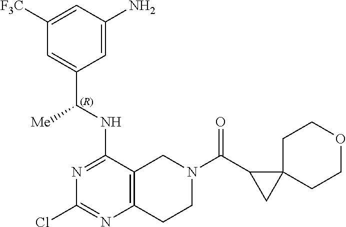 | 510.6 |
| Example 198. | 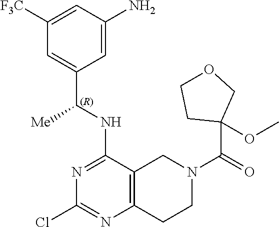 | 500.5 |
| Example 199. | 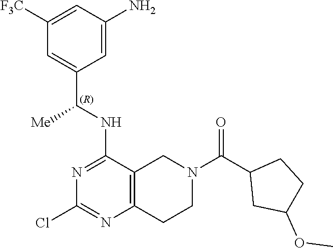 | 498.6 |
| Example 200 | 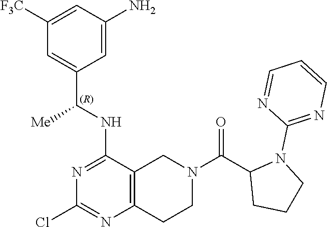 | 547.6 |
| Example 201. | 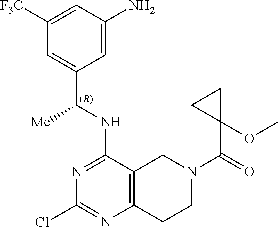 | 470.6 |
| Example 202. | 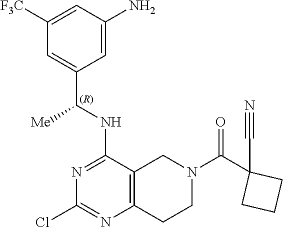 | 479.5 |
| Example 203. | 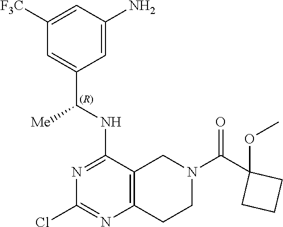 | 484.5 |
| Example 204. | 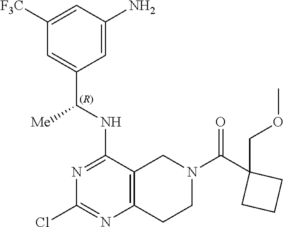 | 498.6 |
| Example 205. | 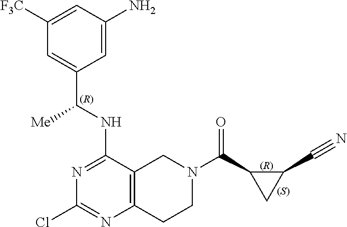 | 465.4 |
| Example 206. | 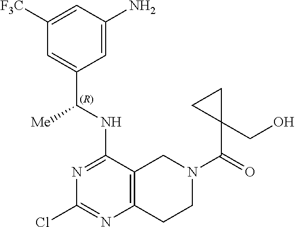 | 470.5 |
| Example 207. | 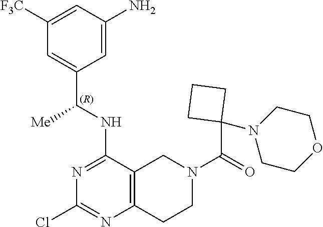 | 539.5 |
| Example 208 | 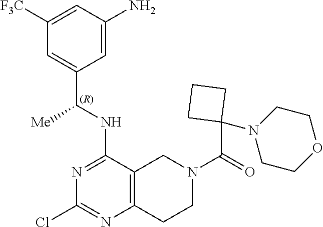 | 595.5 |
| Example 209. | 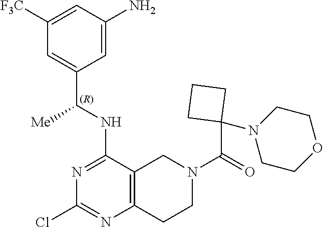 | 454.5 |
| Example 210. | 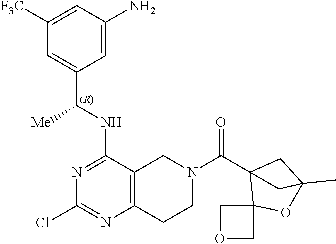 | 538.5 |
| Example 211. | 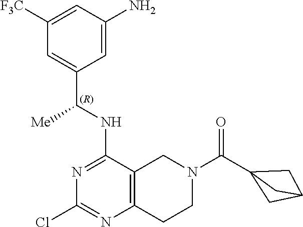 | 466.5 |
| Example 212. | 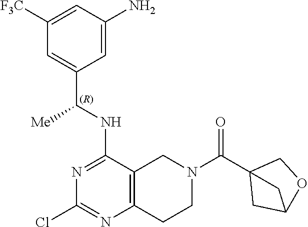 | 482.5 |
| Example 213 | 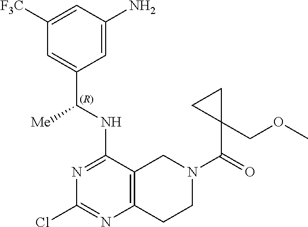 | 484.6 |
| Example 214. | 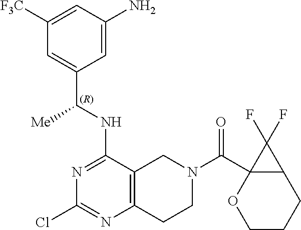 | 532.5 |
| Example 215. | 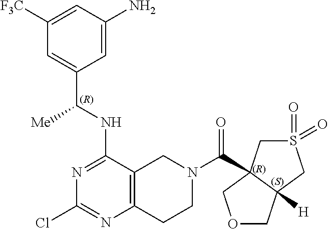 | 560.5 |
| Example 216. | 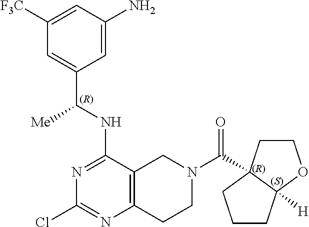 | 510.6 |
| Example 217. | 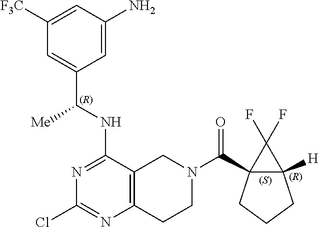 | 518.5 |
| Example 218. | 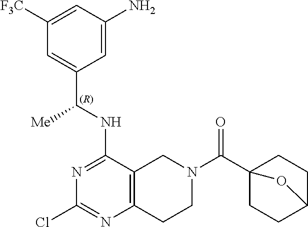 | 496.5 |
| Example 219. | 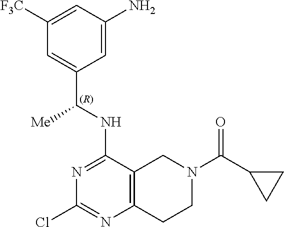 | 44.05 |
| Example 220. | 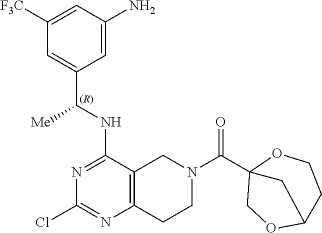 | 512.6 |
| Example 221. | 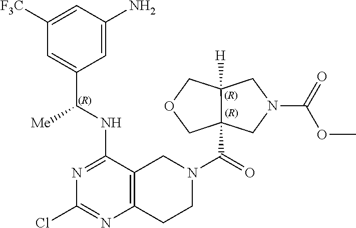 | 569.6 |
| Example 222. | 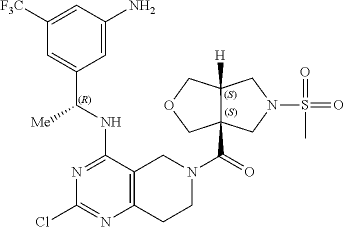 | 589.5 |
| Example 223. | 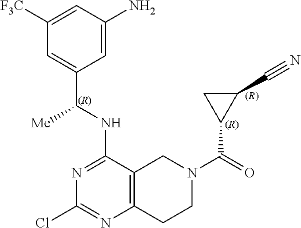 | 465.5 |
| Example 224. | 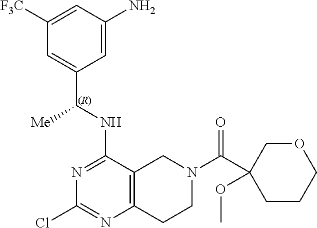 | 514.5 |
| Example 225. | 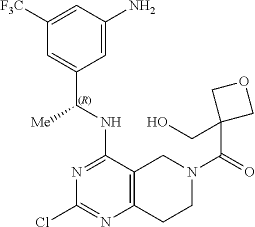 | 486.5 |
| Example 226. | 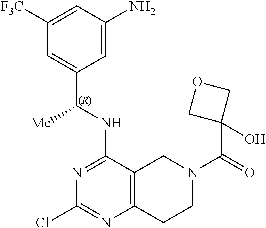 | 472.5 |
| Example 227. | 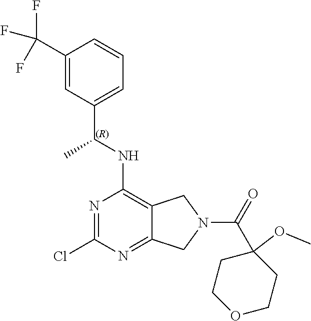 | 485.0 |
| Example 228. | 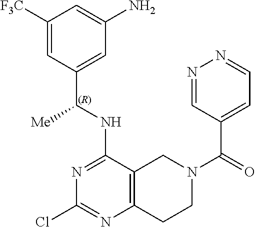 | 477.9 |
| Example 229. | 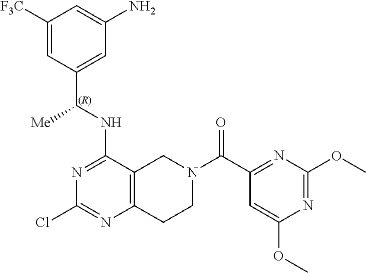 | 537.9 |
| Example 230. | 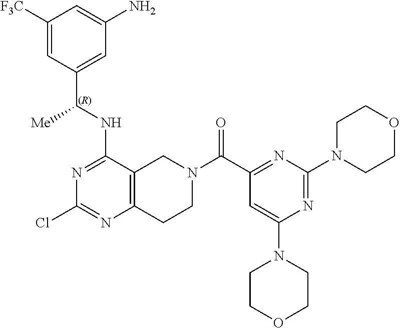 | 648.1 |
| Example 231. | 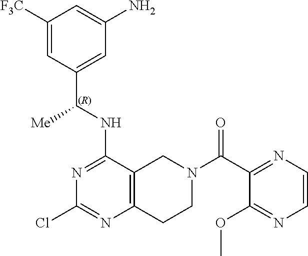 | 507.9 |
| Example 232. | 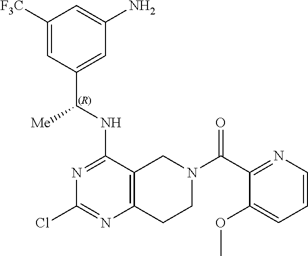 | 506.9 |
| Example 233. | 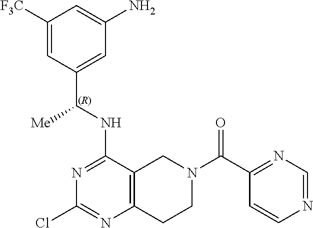 | 478 |
| Example 234. | 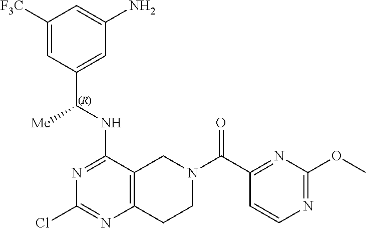 | 507.9 |
| Example 235. | 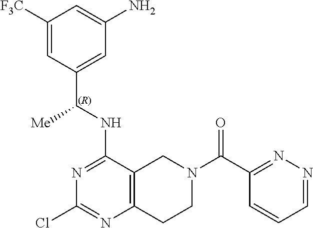 | 477.9 |
| Example 236. | 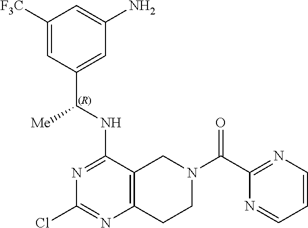 | 477.9 |
| Example 237. | 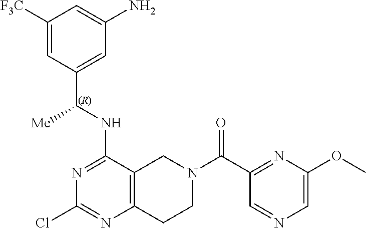 | 507.9 |
| Example 238. | 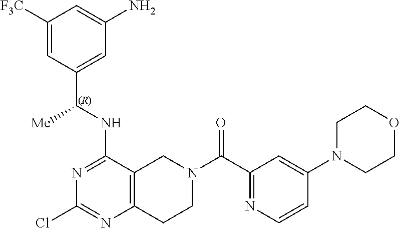 | 562.0 |
| Example 239. | 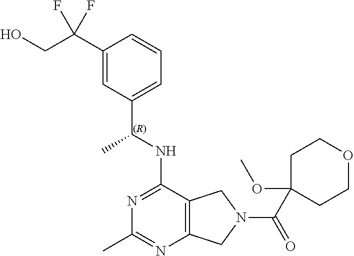 | 477.2 |
| Example 240. | 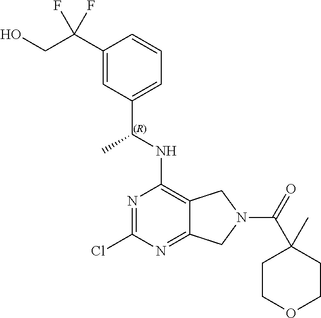 | 481.1 |
| Example 241. | 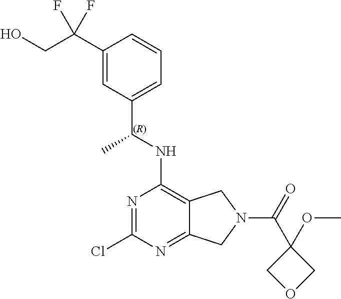 | 469.0 |
| Example 242. | 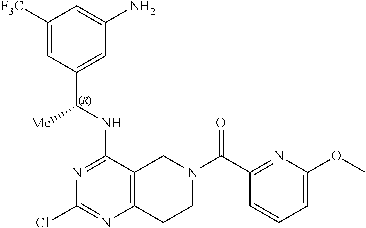 | 507.5 |
| Example 243. | 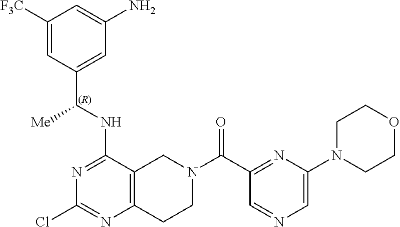 | 563.6 |
| Example 244. | 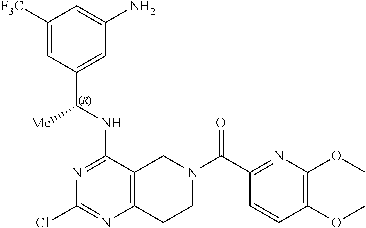 | 537.5 |
| Example 245. | 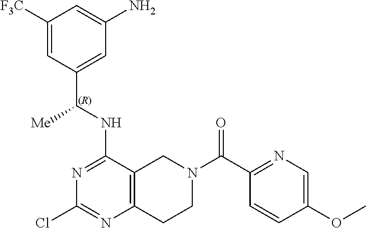 | 507.5 |
| Example 246. | 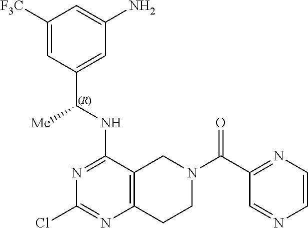 | 478.4 |
| Example 247. | 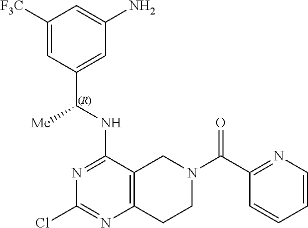 | 477.4 |
| Example 248. | 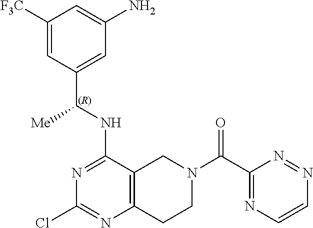 | 479.5 |
| Example 249. | 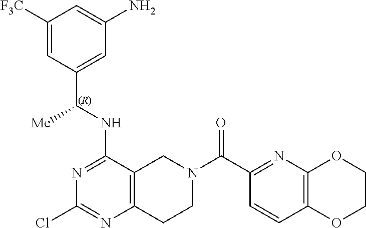 | 535.5 |
| Example 250. | 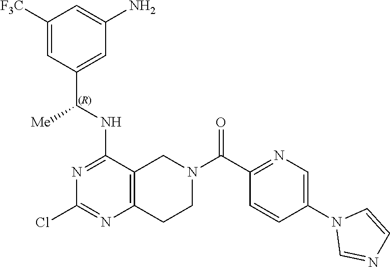 | 543.6 |
| Example 251. | 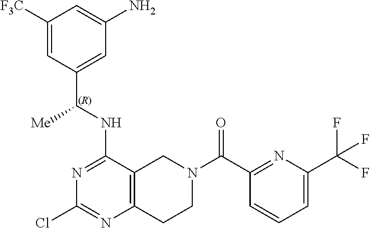 | 545.4 |
| Example 252. | 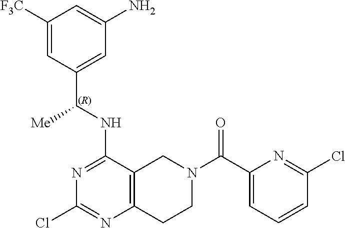 | 511.3 |
| Example 253. | 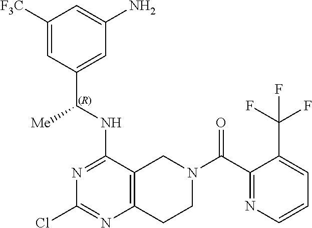 | 545.5 |
| Example 254. | 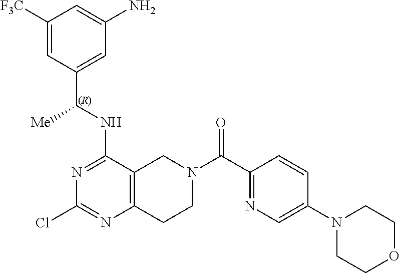 | 562.6 |
| Example 255. | 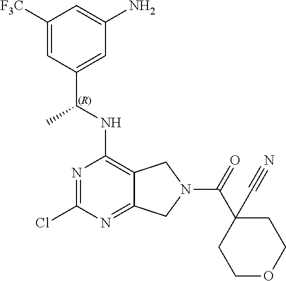 | 495.5 |
| Example 256. | 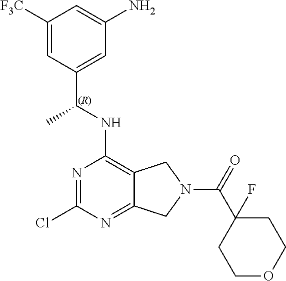 | 488.5 |
| Example 257. | 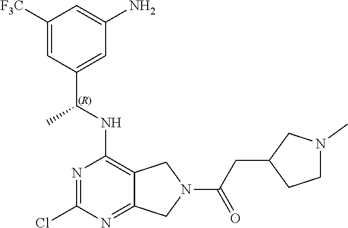 | 483.4 |
| Example 258. | 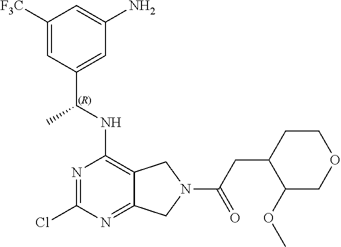 | 514.4 |
| Example 259. | 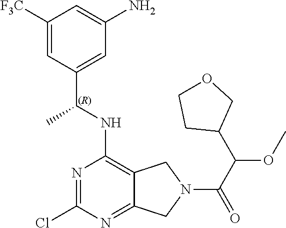 | 500.4 |
| Example 260. | 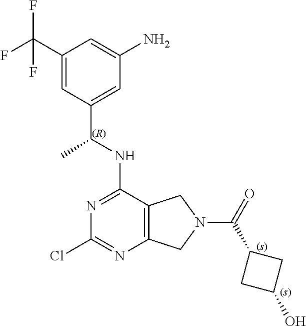 | 456.4 |
| Example 261. | 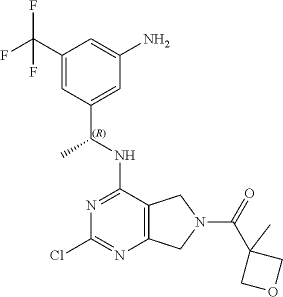 | 456.5 |
| Example 262. | 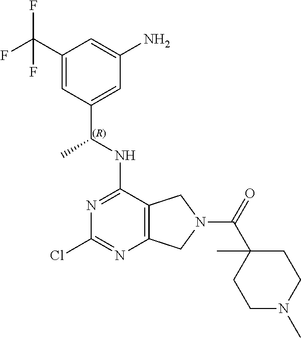 | 497.5 |
| Example 263. | 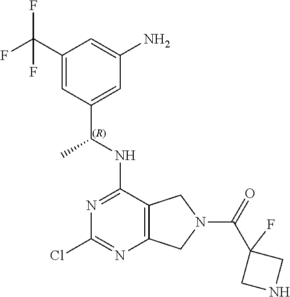 | 459.4 |
| Example 264. | 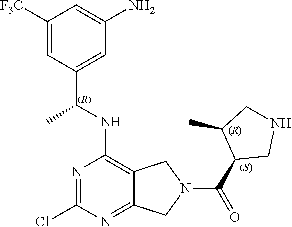 | 469.5 |
| Example 265. | 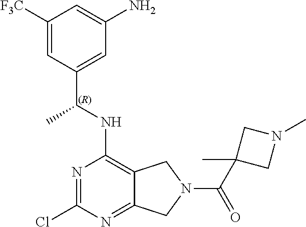 | 469.5 |
| Example 266. | 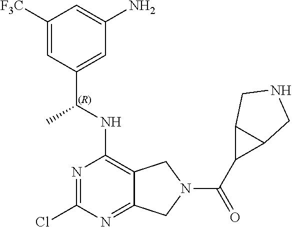 | 467.5 |
| Example 267. | 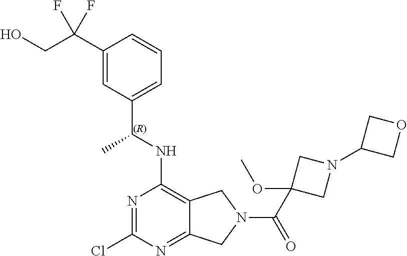 | 524.2 |
| Example 268. | 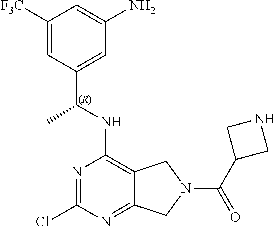 | 441.4 |
| Example 269. | 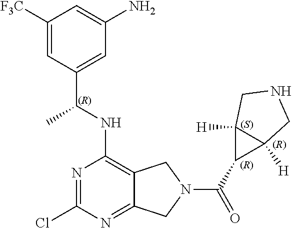 | 467.4 |
| Example 270. | 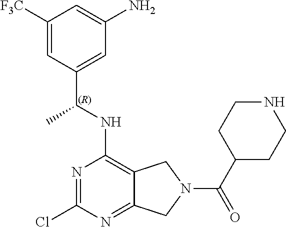 | 469.5 |
| Example 271. | 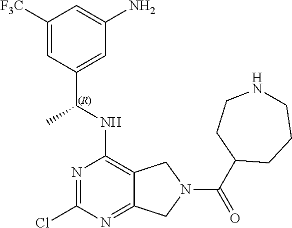 | 479.5 |
| Example 272. | 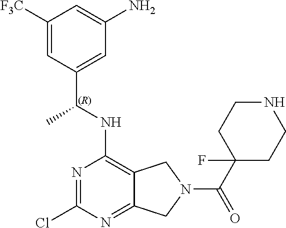 | 487.4 |
| Example 273. | 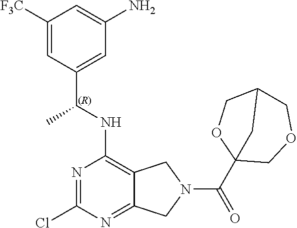 | 498.5 |
| Example 274. | 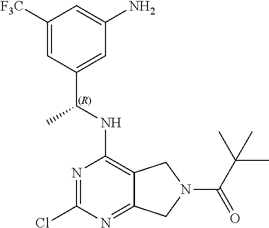 | 442.4 |
| Example 275. | 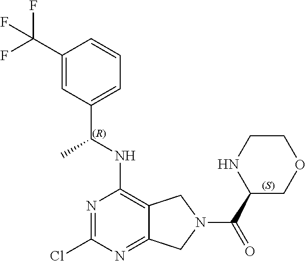 | 456.4 |
| Example 276. | 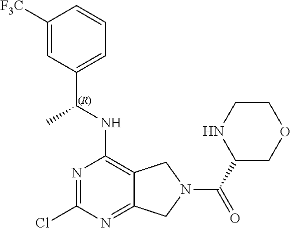 | 456.4 |
| Example 277. | 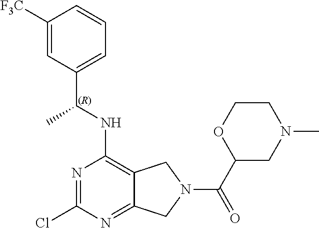 | 470.4 |
| Example 278. | 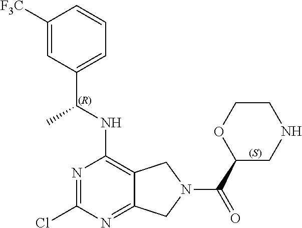 | 456.4 |
| Example 279. | 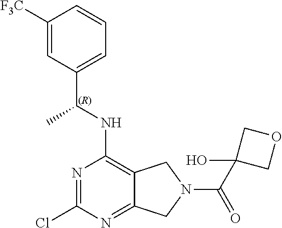 | 443.5 |
| Example 280. | 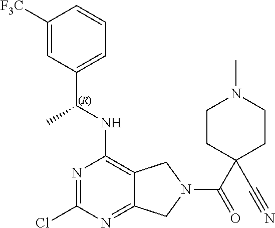 | 493.5 |
| Example 281. | 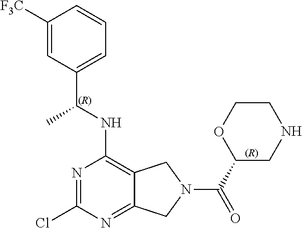 | 456.5 |
| Example 282. | 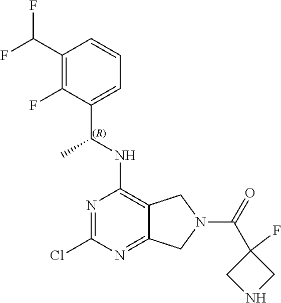 | 444.1 |
| Example 283. | 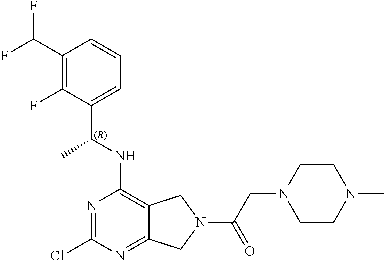 | 483.5 |
| Example 284. | 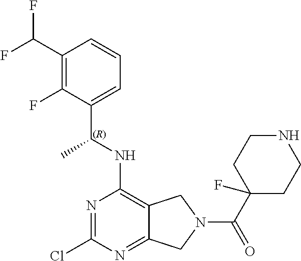 | 472.5 |
| Example 285. | 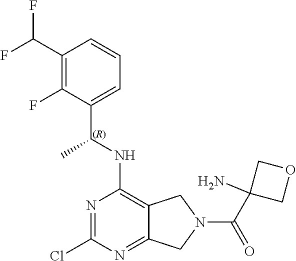 | 442.4 |
| Example 286. | 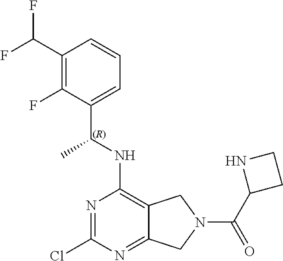 | 426.4 |
| Example 287. | 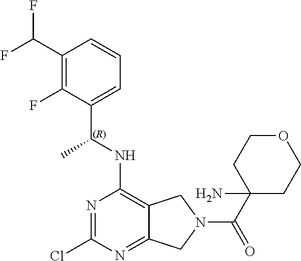 | 470.4 |
| Example 288. | 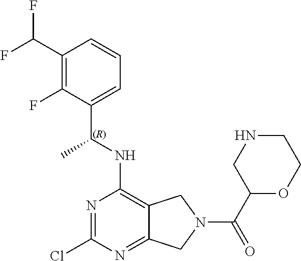 | 456.4 |
| Example 289. | 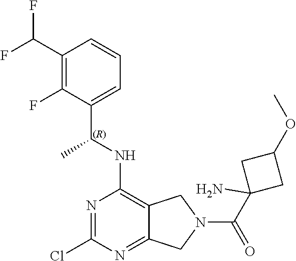 | 470.4 |
| Example 290. | 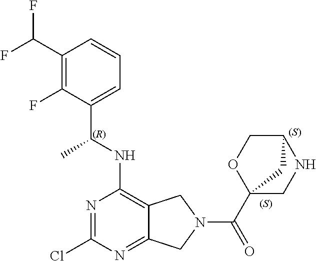 | 468.4 |
| Example 291. | 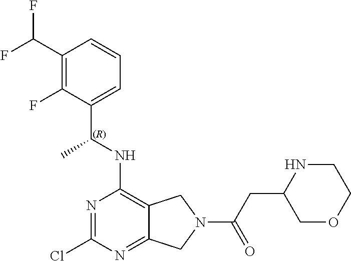 | 470.4 |
| Example 292. | 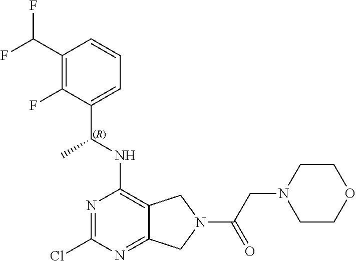 | 470.4 |
| Example 293. | 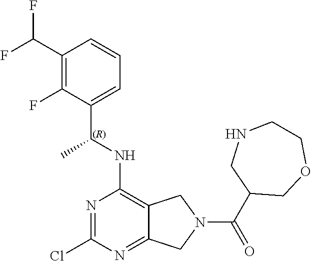 | 470.4 |
| Example 294. | 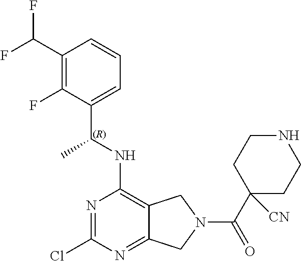 | 479.5 |
| Example 295. | 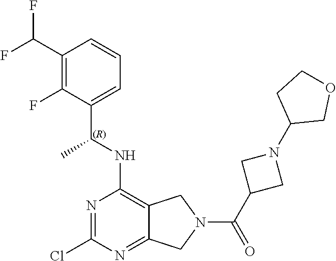 | 496.6 |
| Example 296. | 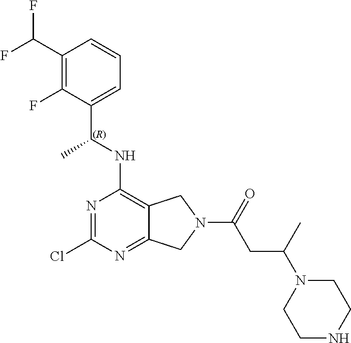 | 498.3 |
| Example 297. | 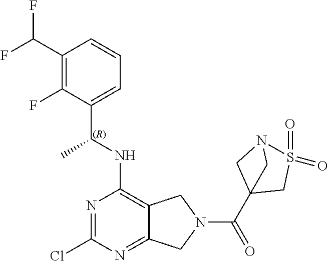 | 502.5 |
| Example 298. | 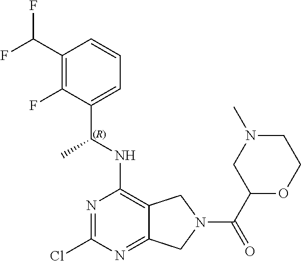 | 470.4 |
| Example 299. | 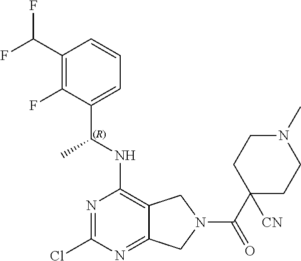 | 493.6 |
| Example 300. | 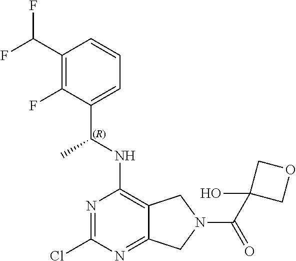 | 443.5 |
| Example 301. | 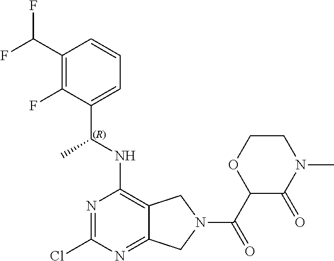 | 484.4 |
| Example 302. | 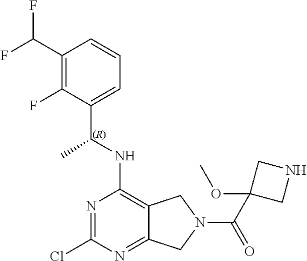 | 456.5 |
| Example 303. | 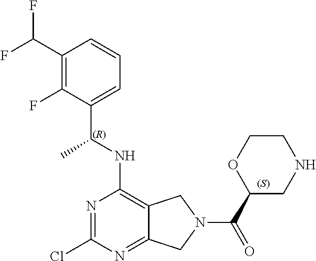 | 456.3 |
| Example 304. | 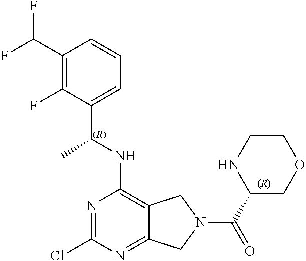 | 456.5 |
| Example 305. | 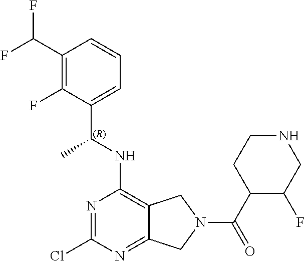 | 472.5 |
| Example 306. | 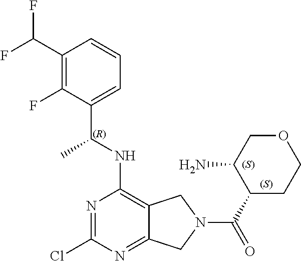 | 470.4 |
| Example 307. | 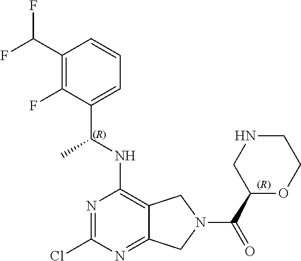 | 456.4 |
| Example 308. | 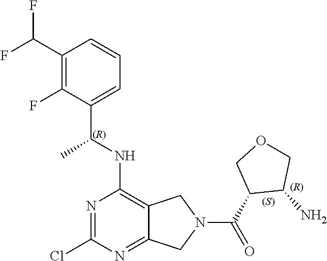 | 456.5 |
| Example 309. | 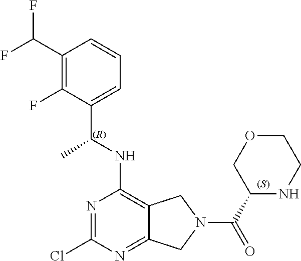 | 456.4 |
| Example 310. | 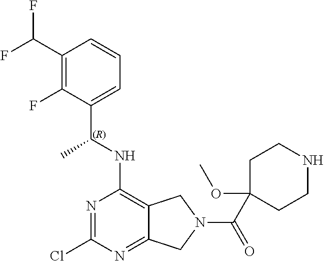 | 484.5 |
| Example 311. | 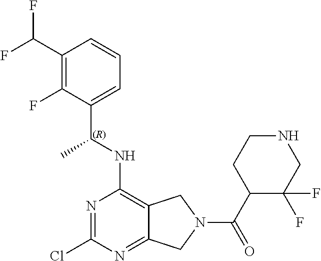 | 490.4 |
| Example 312. | 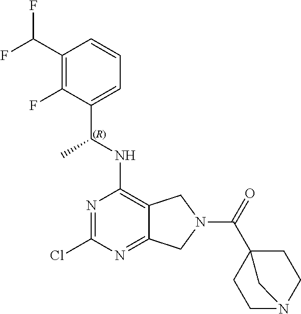 | 466.4 |
| Example 313. | 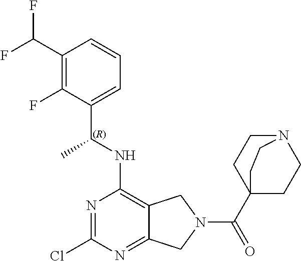 | 480.5 |
| Example 314. | 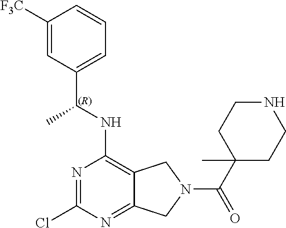 | 468.5 |
| Example 315. | 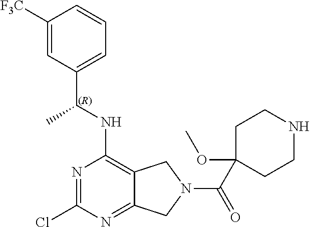 | 484.5 |
| Example 316. | 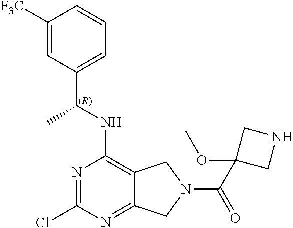 | 456.4 |
| Example 317. | 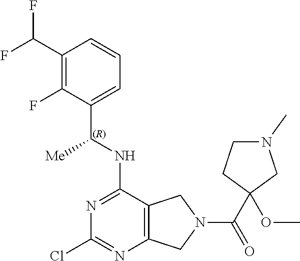 | 458.5 |
| Example 318. | 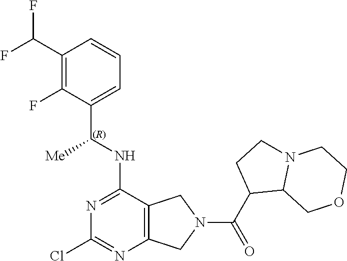 | 496.4 |
| Example 319. | 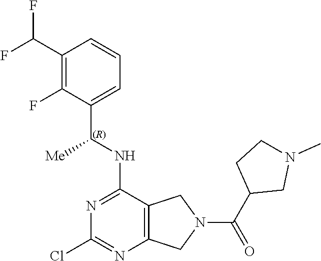 | 454.5 |
| Example 320. | 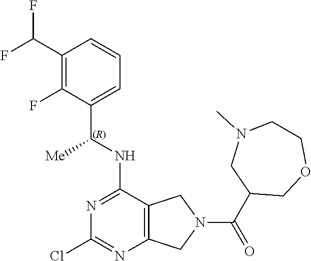 | 484.4 |
| Example 321. | 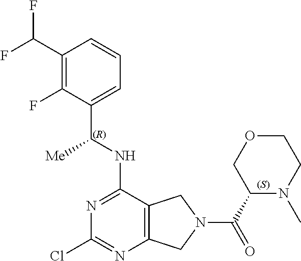 | 470.3 |
| Example 322. | 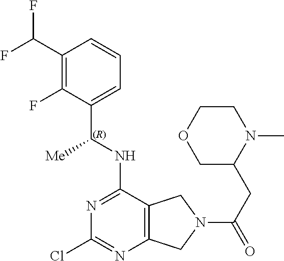 | 484.4 |
| Example 323. | 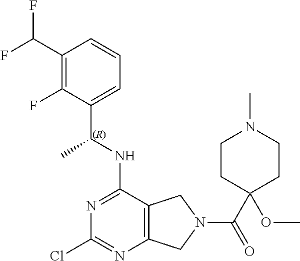 | 497.7 |
| Example 324. | 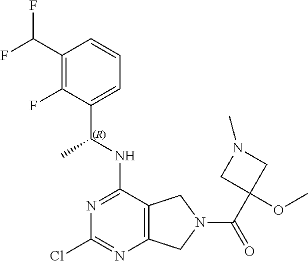 | 469.9 |
| Example 325. | 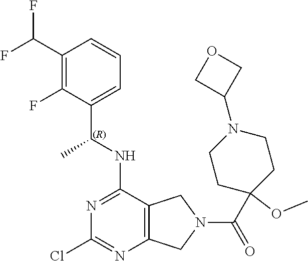 | 540.0 |
| Example 326. | 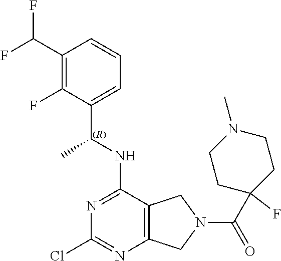 | 485.9 |
| Example 327. | 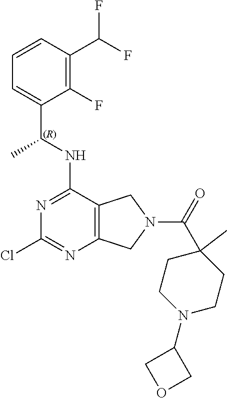 | 524.0 |
| Example 328. | 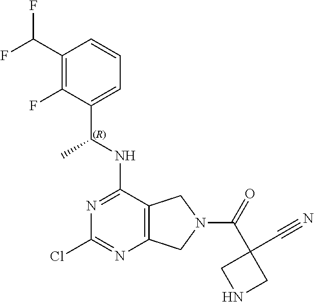 | 451.0 |
| Example 329. | 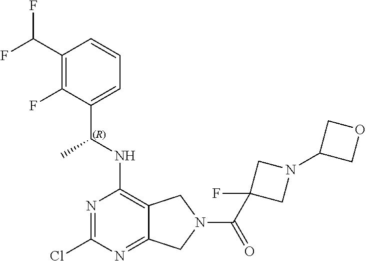 | 500.2 |
| Example 330. | 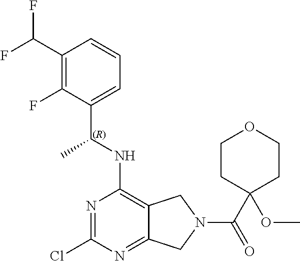 | 485.0 |
| Example 331. | 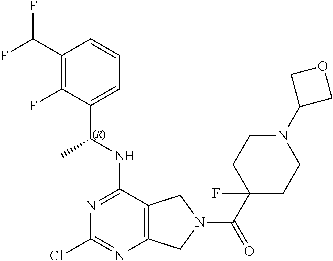 | 528.0 |
| Example 332. | 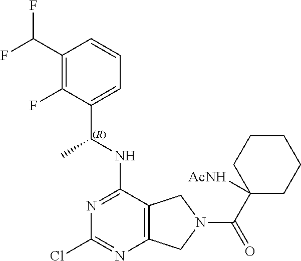 | 510.5 |
| Example 333. | 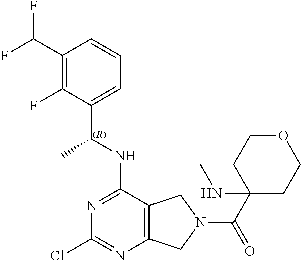 | 484.4 |
| Example 334. | 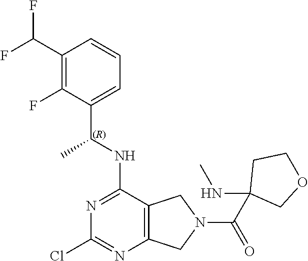 | 470.4 |
| Example 335. | 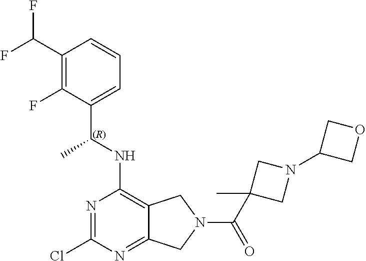 | 496.2 |
| Example 336. | 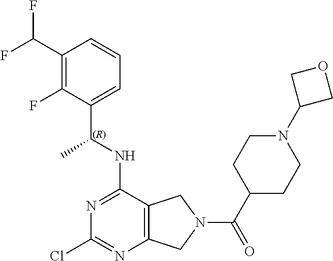 | 510.0 |
| Example 337. | 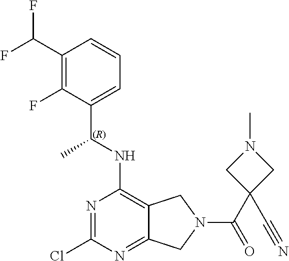 | 464.9 |
| Example 338. | 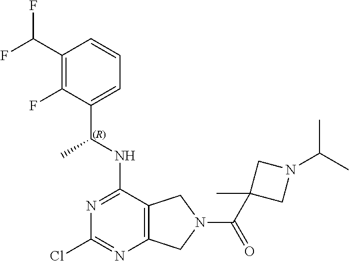 | 481.7 |
| Example 339. | 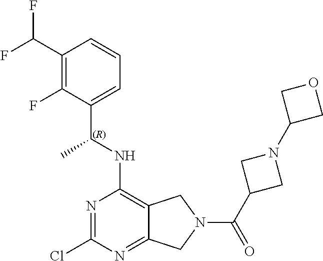 | 482.2 |
| Example 340. | 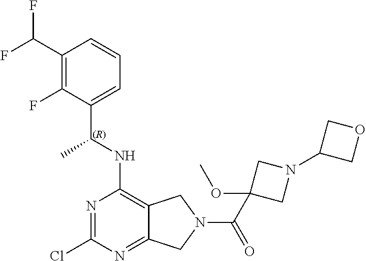 | 512.2 |
| Example 341. | 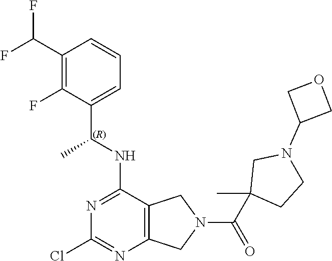 | 510.2 |
| Example 342. | 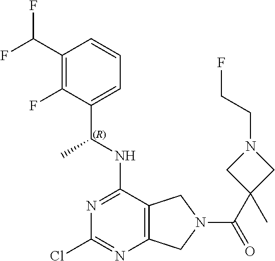 | 486.2 |
| Example 343. | 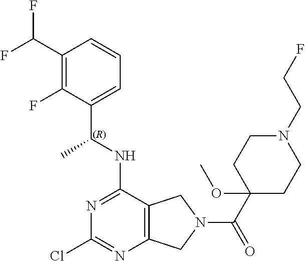 | 530.3 |
| Example 344. | 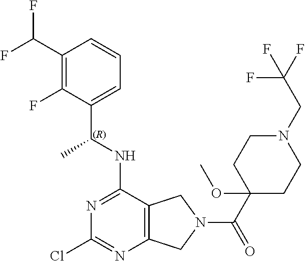 | 566.2 |
| Example 345. | 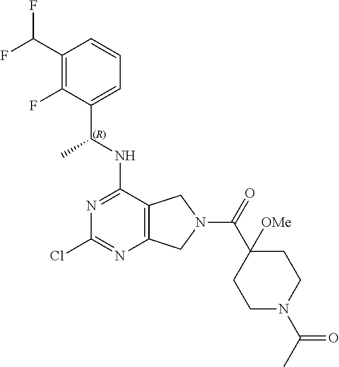 | 526.5 |
| Example 346. | 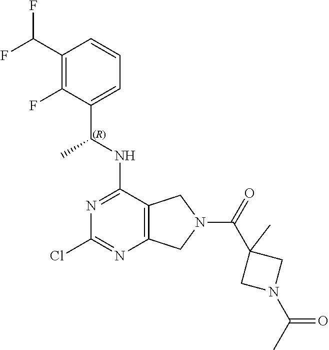 | 482.5 |
| Example 347. | 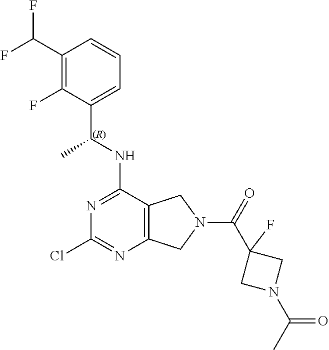 | 486.4 |
| Example 489. | 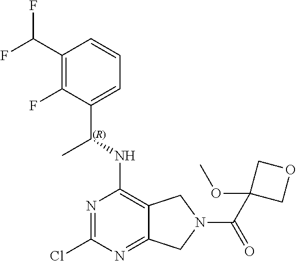 | 457.1 |

### Example 109. Synthesis of [4-[[(1R)-1-[3-amino-5-(trifluoromethyl)phenyl]ethyl]amino]-2-methyl-5,7-dihydropyrrolo[3,4-d]pyrimidin-6-yl]-(1-methyl-4-piperidyl)methanone

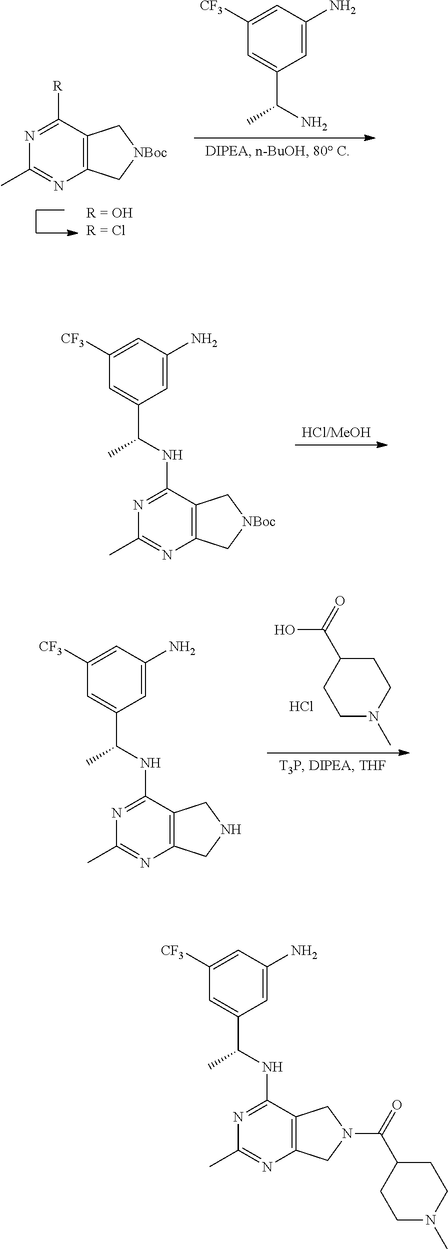

### Step 1.

To a mixture of tert-butyl 4-hydroxy-2-methyl-5,7-dihydropyrrolo[3,4-d]pyrimidine-6-carboxylate (500 mg, 2.0 mmol) and Ph₃P (1.04 g, 3.98 mmol) in DCE (5 mL) was added CCl₄ (574 μL, 5.97 mmol). The mixture was heated to 70° C. and stirred for 3 h, then the solvent was concentrated under reduced pressure and the residue was purified by column chromatography to give tert-butyl 4-chloro-2-methyl-5,7-dihydropyrrolo[3,4-d]pyrimidine-6-carboxylate (240 mg, 45% yield). LCMS (ESI): m/z: [M+H] calculated for C₁₂H₁₇ClN₃O₂: 270.09. found 270.0; H NMR (400 MHz, METHANOL-d₄) δ ppm 4.66 (d, J=4.0 Hz, 4H), 2.72-2.62 (m, 3H), 1.55-1.50 (m, 9H).

### Step 2.

Tert-butyl 4-[[(1R)-1-[3-amino-5-(trifluoromethyl)phenyl]ethyl]amino]-2-methyl-5,7-dihydropyrrolo[3,4-d]pyrimidine-6-carboxylate was synthesized in a manner similar to tert-butyl 4-[[(1R)-1-[3-amino-5-(trifluoromethyl)phenyl]ethyl]amino]-2-chloro-5,7-dihydropyrrolo[3,4-d]pyrimidine-6-carboxylate except tert-butyl 2,4-dichloro-5,7-dihydropyrrolo[3,4-d]pyrimidine-6-carboxylate was substituted with tert-butyl 4-chloro-2-methyl-5,7-dihydropyrrolo[3,4-d]pyrimidine-6-carboxylate. LCMS (ESI): m/z: [M+H] calculated for C₂₁H₂₇F₃N₅O₂: 438.20. found 438.3; ¹H NMR (400 MHz, METHANOL-d₄) δ 6.91 (d, J=12.6 Hz, 2H), 6.81-6.75 (m, 1H), 5.46-5.31 (m, 1H), 4.54-4.39 (m, 4H), 2.39 (s, 3H), 2.01 (s, 3H), 1.52 (s, 9H).

### Step 3.

N-[(1R)-1-[3-amino-5-(trifluoromethyl)phenyl]ethyl]-2-methyl-6,7-dihydro-SH-pyrrolo[3,4-d]pyrimidin-4-amine HCl salt was synthesized in a manner similar to N-[(1R)-1-[3-amino-5-(trifluoromethyl)phenyl]ethyl]-2-chloro-6,7-dihydro-SH-pyrrolo[3,4-d]pyrimidin-4-amine HCl salt except tert-butyl 4-[[(1R)-1-[3-amino-5-(trifluoromethyl)phenyl]ethyl]amino]-2-chloro-5,7-dihydropyrrolo[3,4-d]pyrimidine-6-carboxylate was substituted with tert-butyl 4-[[(1R)-1-[3-amino-5-(trifluoromethyl)phenyl]ethyl]amino]-2-methyl-5,7-dihydropyrrolo[3,4-d]pyrimidine-6-carboxylate. LCMS (ESI): m/z: [M+H] calculated for C₁₋₆H₁₉F₃N₅: 338.15. found 338.2.

### Step 4.

[4-[[(1R)-1-[3-amino-5-(trifluoromethyl)phenyl]ethyl]amino]-2-methyl-5,7-dihydropyrrolo[3,4-d]pyrimidin-6-yl]-(1-methyl-4-piperidyl)methanone was synthesized in a manner similar to [4-[[(1R)-1-[3-amino-5-(trifluoromethyl)phenyl]ethyl]amino]-2-chloro-5,7-dihydropyrrolo[3,4-d]pyrimidin-6-yl]-(1-methyl-4-piperidyl)methanone except N-[(1R)-1-[3-amino-5-(trifluoromethyl)phenyl]ethyl]-2-chloro-6,7-dihydro-5H-pyrrolo[3,4-d]pyrimidin-4-amine HCl salt was substituted with N-[(1R)-1-[3-amino-5-(trifluoromethyl)phenyl]ethyl]-2-methyl-6,7-dihydro-5H-pyrrolo[3,4-d]pyrimidin-4-amine HCl salt. LCMS (ESI): m/z: [M+H] calculated for C₂₃H₃₀F₃N₆O: 463.24. found 463.2; ¹H NMR (400 MHz, METHANOL-d₄) δ ppm 6.95-6.89 (m, 2H), 6.82-6.76 (m, 1H), 5.46-5.36 (m, 1H), 4.79-4.70 (m, 2H), 4.52 (d, J=7.0 Hz, 2H), 2.97 (t, J=10.0 Hz, 2H), 2.64-2.53 (m, 1H), 2.44-2.38 (m, 3H), 2.29 (s, 3H), 2.18-2.05 (m, 2H), 1.90-1.80 (m, 4H), 1.53 (t, J=6.0 Hz, 3H).

### Example 110. (See also Example 54.) Synthesis of N-[(1R)-1-[3-amino-5-(trifluoromethyl)phenyl]ethyl]-2-chloro-6-(tetrahydrofuran-3-ylmethyl)-5,7-dihydropyrrolo[3,4-d]pyrimidin-4-amine

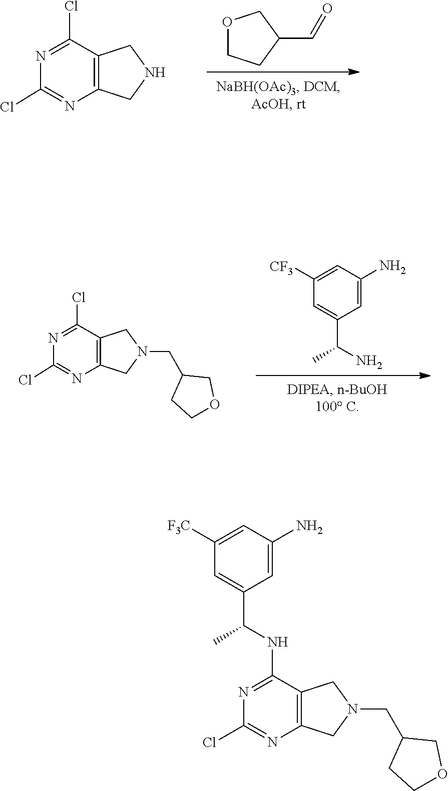 [SMILES: Clc1nc(Cl)c2c(n1)CNC2 ; O=CC1CCOC1 ; Clc1nc(Cl)c2c(n1)CN(CC1CCOC1)C2 ; C[C@@H](N)c1cc(N)cc(C(F)(F)F)c1 ; C[C@@H](Nc1nc(Cl)nc2c1CN(CC1CCOC1)C2)c1cc(N)cc(C(F)(F)F)c1]

### Step 1.

2,4-Dichloro-6-(tetrahydrofuran-3-ylmethyl)-5,7-dihydropyrrolo[3,4-d]pyrimidine was synthesized in a manner similar to 4-chloro-6-(tetrahydrofuran-3-ylmethyl)-5,7-dihydropyrrolo[3,4-d]pyrimidine except 4-chloro-6,7-dihydro-5H-pyrrolo[3,4-d]pyrimidine TFA salt was substituted with 2,4-dichloro-6,7-dihydro-5H-pyrrolo[3,4-d]pyrimidine TFA salt. LCMS (ESI): m/z: [M+H] calculated for C₁₁H₁₄Cl₂N₃O: 274.04. found 274.2.

### Step 2.

N-[(1R)-1-[3-amino-5-(trifluoromethyl)phenyl]ethyl]-2-chloro-6-(tetrahydrofuran-3-ylmethyl)-5,7-dihydropyrrolo[3,4-d]pyrimidin-4-amine was synthesized in a similar manner to N-[(1R)-1-[3-amino-5-(trifluoromethyl)phenyl]ethyl]-6-(tetrahydrofuran-3-ylmethyl)-5,7-dihydropyrrolo[3,4-d]pyrimidin-4-amine except 4-chloro-6-(tetrahy drofuran-3-ylmethyl)-5,7-dihydropyrrolo[3,4-d]pyrimidine was substituted with 2,4-dichloro-6-(tetrahydrofuran-3-ylmethyl)-5,7-dihydropyrrolo[3,4-d]pyrimidine. LCMS (ESI): m/z: [M+H] calculated for C₂₀H₂₄ClF₃N₅O: 442.15. found 442.2; ¹H NMR (400 MHz, CDCl₃) δ ppm 6.95 (s, 1H), 6.82 (s, 2H), 5.32 (m, 1H), 4.67-4.79 (m, 1H), 3.72-3.91 (m, 8H), 3.56 (dd, J=8.0, 6.4 Hz, 1H), 2.72 (br d, J=7.6 Hz, 2H), 2.42-2.45 (m, 1H), 2.06-2.07 (m, 1H), 1.60-1.67 (m, 1H), 1.57 (d, J=6.8 Hz, 3H).

### Example 111. Synthesis of N-[(1R)-1-[3-amino-5-(trifluoromethyl)phenyl]ethyl]-2-methyl-6-(tetrahydrofuran-3-ylmethyl)-5,7-dihydropyrrolo[3,4-d]pyrimidin-4-amine

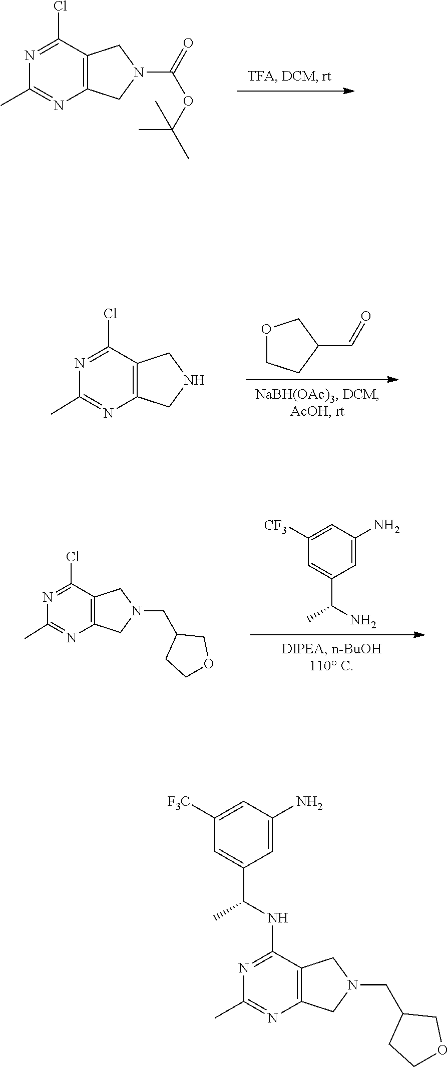 [SMILES: Cc1nc(Cl)c2c(n1)CN(C(=O)OC(C)(C)C)C2 ; Cc1nc(Cl)c2c(n1)CNC2 ; O=CC1CCOC1 ; Cc1nc(Cl)c2c(n1)CN(CC1CCOC1)C2 ; C[C@@H](N)c1cc(N)cc(C(F)(F)F)c1 ; Cc1nc2c(c(N[C@H](C)c3cc(N)cc(C(F)(F)F)c3)n1)CN(CC1CCOC1)C2]

### Step 1.

To a mixture of tert-butyl 4-chloro-2-methyl-5,7-dihydropyrrolo[3,4-d]pyrimidine-6-carboxylate (600 mg, 2.2 mmol) in DCM (3 mL) was added TFA (3 mL). The mixture was stirred at rt for 1 h, then the solvent was concentrated under reduced pressure to give 4-chloro-2-methyl-6,7-dihydro-5H-pyrrolo[3,4-d]pyrimidine TFA salt (630 mg, 100% yield). LCMS (ESI): m/z: [M+H] calculated for C₇H9ClN₃: 170.04. found 170.2.

### Step 2.

4-chloro-2-methyl-6-(tetrahydrofuran-3-ylmethyl)-5,7-dihydropyrrolo[3,4-d]pyrimidine was synthesized in a manner similar to 4-chloro-6-(tetrahydrofuran-3-ylmethyl)-5,7-dihydropyrrolo[3,4-d]pyrimidine except 4-chloro-6,7-dihydro-5H-pyrrolo[3,4-d]pyrimidine TFA salt was substituted with 4-chloro-2-methyl-6,7-dihydro-5H-pyrrolo[3,4-d]pyrimidine TFA salt. LCMS (ESI): m/z: [M+H] calculated for C₂H₁₇ClN₃O: 254.10. found 254.2.

### Step 3.

N-[(1R)-1-[3-amino-5-(trifluoromethyl)phenyl]ethyl]-2-methyl-6-(tetrahydrofuran-3-ylmethyl)-5,7-dihydropyrrolo[3,4-d]pyrimidin-4-amine was synthesized in a similar manner to N-[(1R)-1-[3-amino-5-(trifluoromethyl)phenyl]ethyl]-6-(tetrahydrofuran-3-ylmethyl)-5,7-dihydropyrrolo[3,4-d]pyrimidin-4-amine except 4-chloro-6-(tetrahydrofuran-3-ylmethyl)-5,7-dihydropyrrolo[3,4-d]pyrimidine was substituted with 4-chloro-2-methyl-6-(tetrahydrofuran-3-ylmethyl)-5,7-dihydropyrrolo[3,4-d]pyrimidine. LCMS (ESI): m/z: [M+H] calculated for C₂₁H₂₇F₃N₅O: 422.21. found 422.2; H NMR (400 MHz, METHANOL-d₄) δ ppm 6.90 (d, J=8.0 Hz, 2H), 6.79 (s, 1H), 5.42-5.32 (m, 1H), 3.96-3.73 (m, 8H), 3.57-3.51 (m, 1H), 2.82-2.75 (m, 2H), 2.58-2.46 (m, 1H), 2.40-2.35 (m, 3H), 2.17-2.08 (m, 1H), 1.74-1.63 (m, 1H), 1.51 (d, J=7.0 Hz, 3H).

### Example 112. Synthesis of N-[(1R)-1-[3-amino-5-(trifluoromethyl)phenyl]ethyl]-2-methyl-7-(morpholine-4-carbonyl)-5H,6H,7H,8H,9H-pyrimido[4,5-d]-azepin-4-amine

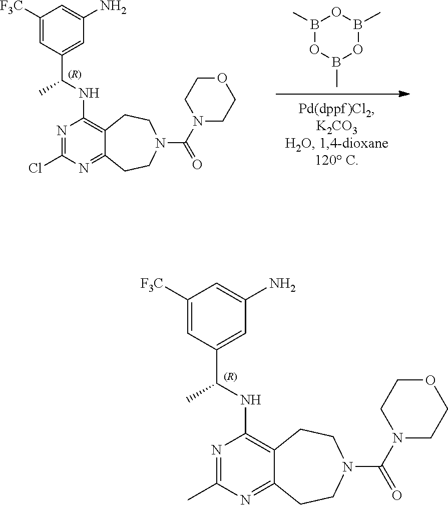

### Step 1.

A microwave vessel was charged with N-[(1R)-1-[3-amino-5-(trifluoromethyl)phenyl]ethyl]-2-chloro-7-(morpholine-4-carbonyl)-5H,6H,7H-8H,9H-pyrimido[4,5-d]-azepin-4-amine (35 mg, 0.07 mmol), trimethylboroxine, 50% solution in THF (118 μL, 0.42 mmol) and K₂CO₃ (20 mg, 0.14 mmol) in 1,4-dioxane (1.3 mL) and H₂O (0.4 mL). The mixture was degassed and Pd(dppf)Cl₂ (5 mg, 0.01 mmol) was added, then the mixture heated to 120° C. and stirred for 40 min under microwave irradiation. The mixture was purified by prep-HPLC to give N-[(1R)-1-[3-amino-5-(trifluoromethyl)phenyl]ethyl]-2-methyl-7-(morpholine-4-carbonyl)-5H,6H,7H,8H,9H-pyrimido[4,5-d]-azepin-4-amine (15 mg, 43% yield). LCMS (ESI): m/z: [M+H] calculated for C₂₃H₂₉F₃N₆O₂: 478.52. found 479.22; ¹H NMR (300 MHz, METHANOL-d₄) δ ppm 6.92 (s, 1H), 6.90 (s, 1H), 6.77 (s, 1H), 5.38 (q, J=7.2 Hz, 1H), 3.65 (t, J=4.7 Hz, 4H), 3.53 (dd, J=6.5, 4.4 Hz, 4H), 3.21 (t, J=4.7 Hz, 4H), 3.03-2.93 (m, 2H), 2.92-2.78 (m, 2H), 2.32 (s, 3H), 1.52 (d, J=7.1 Hz, 3H).

### Example 113. Synthesis of N-[(1R)-1-[3-amino-5-(trifluoromethyl)phenyl]ethyl]-2-chloro-7-(oxolan-3-ylmethyl)-5H,6H,7H,8H,9H-pyrimido[4,5-d]aze-pin-4-amine

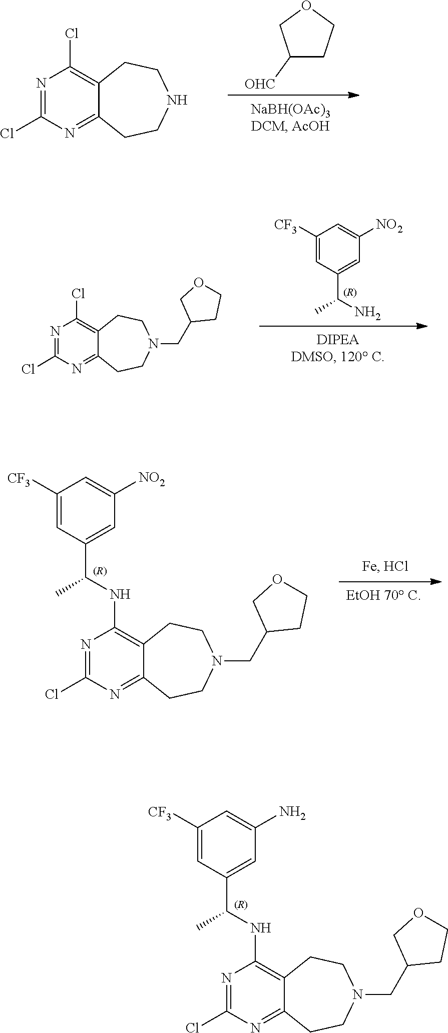

### Step 1.

A mixture of 2,4-dichloro-5H,6H,7H,8H,9H-pyrimido[4,5-d]azepine (300 mg, 1.38 mmol), tetrahydrofuran-3-carboxaldehyde, 50 wt. % in H₂O (747 μL, 4.13 mmol) and AcOH (826 mg, 787 μL, 13.76 mmol) in DCM (15.3 mL) was heated to 60° C. in a sealed tube and stirred for 1 h. The mixture was cooled to 0° C. and NaBH(OAc)₃ (583 mg, 2.75 mmol) was added portion-wise. The mixture was slowly heated to rt over 2.5 h, then quenched with 1M NaOH, and extracted with EtOAc. The combined organic layers were washed with NaHCO₃, dried over anhydrous Na₂SO₄, and filtered. The solvent was concentrated under reduced pressure and the residue was purified by column chromatography to give 2,4-dichloro-7-(oxolan-3-ylmethyl)-5H,6H,7H,8H,9H-pyrimido[4,5-d]azepine (258 mg, 62% yield). LCMS (ESI): m/z: [M+H] calculated for C₁₃H₁₇Cl₂N₃O: 302.2. found 302.55; H NMR (300 MHz, CDCl₃) δ ppm 3.92-3.81 (m, 1H), 3.75 (q, J=7.7 Hz, 1H), 3.56 (dd, J=9.4, 3.7 Hz, 1H), 3.19-3.03 (m, 4H), 2.77-2.58 (m, 3H), 2.44 (d, J=12.4 Hz, 3H), 2.08-1.93 (m, 1H), 1.69-1.56 (m, 1H), 1.56 (s, 2H).

### Step 2.

A mixture of 2,4-dichloro-7-(oxolan-3-ylmethyl)-5H,6H,7H,8H,9H-pyrimido[4,5-d]azepine (255 mg, 0.85 mmol) and (1R)-1-[3-nitro-5-(trifluoromethyl)phenyl]ethan-1-amine HCl salt (252 mg, 0.92 mmol) in anhydrous DMSO (7.7 mL) was purged with Ar. DIPEA (488 μL, 3.37 mmol) was added and the mixture was heated to 120° C. for 2 h under microwave irradiation. H₂O and Et₂O were added and the aqueous layer extracted with Et₂O (×2). The combined organic layers were washed with H₂O, dried over anhydrous Na₂SO₄, and filtered. The solvent was concentrated under reduced pressure and the residue was purified by column chromatography to give 2-chloro-N-[(1R)-1-[3-nitro-5-(trifluoromethyl)phenyl]ethyl]-7-(oxolan-3-ylmethyl)-5H,6H,7H,8H,9H-pyrimido[4,5-d]azepin-4-amine (152 mg, 36% yield). LCMS (ESI): m/z: [M+H] calculated for C₂₂H₂₅ClF₃N5O₃: 499.92. found 501.10; ¹H NMR (300 MHz, CDCl₃) δ ppm 8.43 (s, 1H), 8.38 (s, 1H), 8.03 (s, 1H), 5.39 (p, J=6.8 Hz, 1H), 5.08 (s, 1H), 3.96-3.82 (m, 2H), 3.75 (q, J=7.7 Hz, 1H), 3.57 (dd, J=8.2, 4.4 Hz, 1H), 2.96 (s, 2H), 2.70 (s, 4H), 2.62 (s, 2H), 2.49 (s, 3H), 2.11-1.98 (m, 1H), 1.67 (d, J=7.0 Hz, 3H).

### Step 3.

Fe powder (93 mg, 1.67 mmol) and 1M HCl (1.22 mL, 1.22 mmol) were added to a mixture of 2-chloro-N-[(1R)-1-[3-nitro-5-(trifluoromethyl)phenyl]ethyl]-7-(oxolan-3-ylmethyl)-5H,6H,7H,8H,9H-pyrimido[4,5-d]azepin-4-amine (152 mg, 0.3 mmol) in EtOH (3 mL). The mixture was heated to 70° C. and stirred overnight. The mixture was filtered through Celite®, the filter cake was washed with MeOH, and the solvent was concentrated under reduced pressure. The residue was dissolved in EtOAc, washed with NaHCO₃ and the aqueous layer extracted with EtOAc (×3). The combined organic layers were dried over anhydrous Na₂SO₄, the solvent was concentrated under reduced pressure and the residue was purified by prep-HPLC to give N-[(1R)-1-[3-amino-5-(trifluoromethyl)phenyl]ethyl]-2-chloro-7-(oxolan-3-ylmethyl)-5H,6H,7H,8H,9H-pyrimido[4,5-d]azepin-4-amine (74 mg, 52% yield). LCMS (ESI): m/z: [M+H] calculated for C₂₂H₂₇ClF₃N₅O: 469.94. found 470.17; ¹H NMR (300 MHz, METHANOL-d₄) δ ppm 6.90 (d, J=8.4 Hz, 2H), 6.80 (s, 1H), 5.29 (q, J=7.0 Hz, 1H), 3.93-3.78 (m, 2H), 3.74 (q, J=7.7 Hz, 1H), 3.57-3.48 (m, 1H), 2.88 (t, J=5.1 Hz, 2H), 2.85-2.75 (m, 2H), 2.74-2.59 (m, 4H), 2.57-2.43 (m, 3H), 2.13-1.98 (m, 1H), 1.64 (dq, J=13.7, 7.7 Hz, 1H), 1.52 (d, J=7.1 Hz, 3H).

### Example 114. Synthesis of N-[(1R)-1-[3-amino-5-(trifluoromethyl)phenyl]ethyl]-2-chloro-7-(oxolane-3-carbonyl)-5H,6H,7H,8H,9H-pyrimido[4,5-d]azepin-4-amine

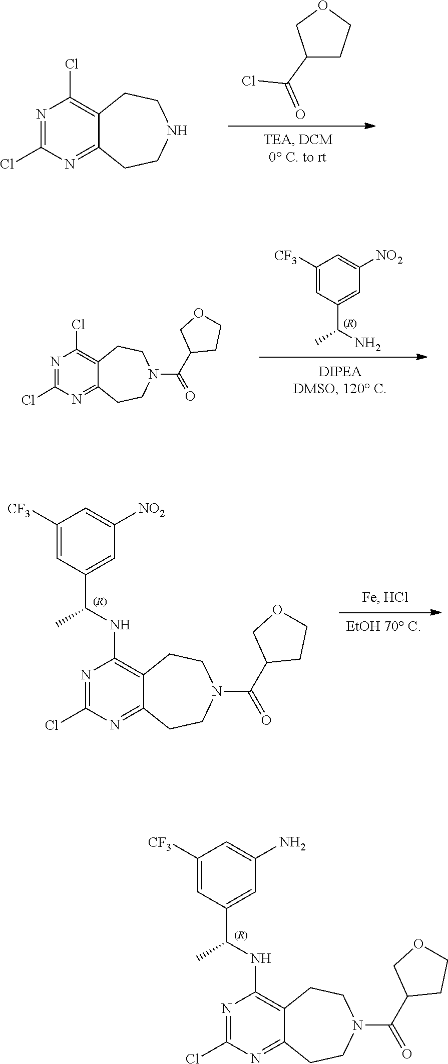

### Step 1.

Tetrahydrofuran-3-carbonyl chloride (106 mg, 0.79 mmol) in DCM (0.4 mL) was added dropwise to a mixture of 2,4-dichloro-5H,6H,7H,8H,9H-pyrimido[4,5-d]azepine HCl salt (0.2 g, 0.79 mmol) in DCM (6 mL) and TEA (0.49 mL, 3.54 mmol) at 0° C. under an atmosphere of Ar. The mixture was stirred at rt overnight, then diluted with DCM and washed with H₂O and brine. The combined aqueous layers were extracted with DCM (×2), and the combined organic layers were dried over anhydrous Na₂SO₄ and filtered. The solvent was concentrated under reduced pressure and the residue was purified by column chromatography to give 2,4-dichloro-7-(oxolane-3-carbonyl)-5H,6H,7H,8H,9H-pyrimido[4,5-d]azepine (122 mg, 49% yield). LCMS (ESI): m/z: [M+H] calculated for C₁₃H₁₅Cl₂N₃O₂: 316.18. found 316.55; H NMR (300 MHz, CDCl₃) δ ppm 3.89 (td, J=8.1, 5.3 Hz, 1H), 3.80-3.70 (m, 2H), 3.73-3.62 (m, 4H), 3.40 (m, 1H), 3.22-3.15 (m, 1H), 3.11 (q, J=5.3 Hz, 2H), 3.06-2.99 (m, 1H), 2.52 (d, J=2.1 Hz, 1H), 2.10-1.99 (m, 1H), 1.98-1.90 (m, 1H).

### Step 2.

2-chloro-N-[(1R)-1-[3-nitro-5-(trifluoromethyl)phenyl]ethyl]-7-(oxolane-3-carbonyl)-5H,6H,7H,8H,9H-pyrimido-[4,5-d]azepin-4-amine was synthesized in a manner similar to 2-chloro-N-[(1R)-1-[3-nitro-5-(trifluoromethyl)phenyl]ethyl]-7-(oxolan-3-ylmethyl)-5H,6H,7H,8H,9H-pyrimido[4,5-d]azepin-4-amine except 2,4-dichloro-7-(oxolan-3-ylmethyl)-5H,6H,7H,8H,9H-pyrimido[4,5-d]azepine was substituted with 2,4-dichloro-7-(oxolane-3-carbonyl)-5H,6H,7H,8H,9H-pyrimido[4,5-d]azepine. LCMS (ESI): m/z: [M+H] calculated for C₂₂H₂₃ClF₃N₅O₄: 513.90. found 515.05; H NMR (300 MHz, CDCl₃) δ ppm 8.44 (s, 1H), 8.39 (s, 1H), 8.03 (d, J=13.1 Hz, 1H), 5.51 (br s, 1H), 5.46-5.34 (m, 1H), 4.07-3.92 (m, 2H), 3.84 (dd, J=27.2, 10.9 Hz, 6H), 3.29-3.13 (m, 3H), 2.74 (s, 2H), 2.10 (dd, J=13.3, 6.0 Hz, 2H), 1.69 (d, J=6.9 Hz, 3H).

### Step 3.

N-[(1R)-1-[3-amino-5-(trifluoromethyl)phenyl]ethyl]-2-chloro-7-(oxolane-3-carbonyl)-5H,6H,7H,8H,9H-pyrimido[4,5-d]azepin-4-amine was synthesized in a manner similar to N-[(1R)-1-[3-amino-5-(trifluoromethyl)phenyl]ethyl]-2-chloro-7-(oxolan-3-ylmethyl)-5H,6H,7H,8H,9H-pyrimido[4,5-d]azepin-4-amine except 2-chloro-N-[(1R)-1-[3-nitro-5-(trifluoromethyl)phenyl]ethyl]-7-(oxolan-3-ylmethyl)-5H,6H,7H,8H,9H-pyrimido[4,5-d]azepin-4-amine was substituted with 2-chloro-N-[(1R)-1-[3-nitro-5-(trifluoromethyl)phenyl]ethyl]-7-(oxolane-3-carbonyl)-5H,6H,7H,8H,9H-pyrimido-[4,5-d]azepin-4-amine. LCMS (ESI): m/z: [M+H] calculated for C₂₂H₂₅ClF₃N5O₂: 483.92. found 484.13; ¹H NMR (300 MHz, METHANOL-d₄) δ ppm 6.98-6.84 (m, 2H), 6.79 (s, 1H), 5.39-5.20 (m, 1H), 3.93 (q, J=7.9 Hz, 1H), 3.88-3.66 (m, 6H), 3.44 (p, J=7.9 Hz, 1H), 3.09-2.93 (m, 2H), 2.84 (dt, J=11.7, 6.4 Hz, 2H), 2.23-1.98 (m, 2H), 1.98-1.84 (m, 1H), 1.61-1.45 (m, 3H).

### Example 115. Synthesis of oxan-4-yl 4-{[(1R)-1-[3-amino-5-(trifluoromethyl)phenyl]ethyl]amino}-2-chloro-5H,6H,7H-pyrrolo[3,4-d]pyrimidine-6-carboxylate

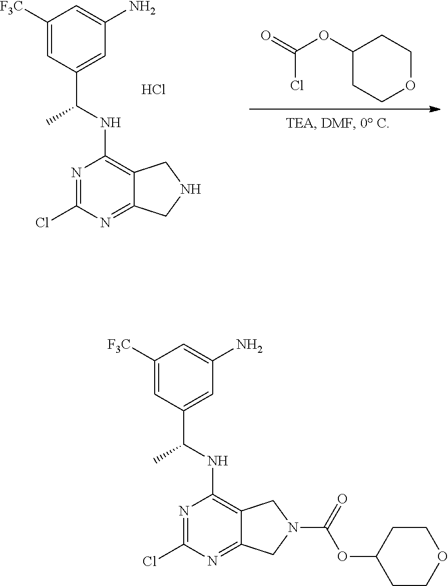 [SMILES: C[C@@H](Nc1nc(Cl)nc2c1CNC2)c1cc(N)cc(C(F)(F)F)c1 ; Cl ; O=C(Cl)OC1CCOCC1 ; C[C@@H](Nc1nc(Cl)nc2c1CN(C(=O)OC1CCOCC1)C2)c1cc(N)cc(C(F)(F)F)c1]

### Step 1.

To a mixture of N-[(1R)-1-[3-amino-5-(trifluoromethyl)phenyl]ethyl]-2-chloro-5H,6H,7H-pyrrolo[3,4-d]pyrimidin-4-amine HCl salt (239 mg, 0.51 mmol) and TEA (355 μL, 2.55 mmol) in DMF (6 mL) at 0° C. under an atmosphere of Ar was added oxan-4-yl chloroformate (84 mg, 0.51 mmol) in DMF (2.4 mL). The mixture was stirred at 0° C. for 1 h, then saturated NH₄Cl added and the mixture extracted with Et₂O/EtOAc (×2). The combined organic layers were washed with NaHCO₃, H₂O and brine, dried over anhydrous Na₂SO₄, and filtered. The solvent was concentrated under reduced pressure and the residue was purified by prep-HPLC to give oxan-4-yl 4-{[(1R)-1-[3-amino-5-(trifluoromethyl)phenyl]ethyl]amino}-2-chloro-5H,6H,7H-pyrrolo[3,4-d]pyrimidine-6-carboxylate (130 mg, 52% yield). LCMS (ESI): m/z: [M+H] calculated for C₂₁H₂₃ClF₃N₅O₃: 485.14. found 486.15; ¹H NMR (300 MHz, DMSO-d₆) δ ppm 8.22-8.11 (m, 1H), 6.82 (s, 1H), 6.76 (s, 1H), 6.70 (s, 1H), 5.57 (s, 2H), 5.25-5.11 (m, 1H), 4.90-4.75 (m, 1H), 4.58-4.35 (m, 4H), 3.88-3.75 (m, 2H), 3.56-3.41 (m, 2H), 1.96-1.81 (m, 2H), 1.65-1.51 (m, 2H), 1.47-1.39 (m, 3H).

### Example 116. Synthesis of N-[(1R)-1-[3-amino-5-(trifluoromethyl)phenyl]ethyl]-2-chloro-6-(oxane-4-carbonyl)-5H,6H,7H-pyrrolo[3,4-d]pyrimidin-4-amine

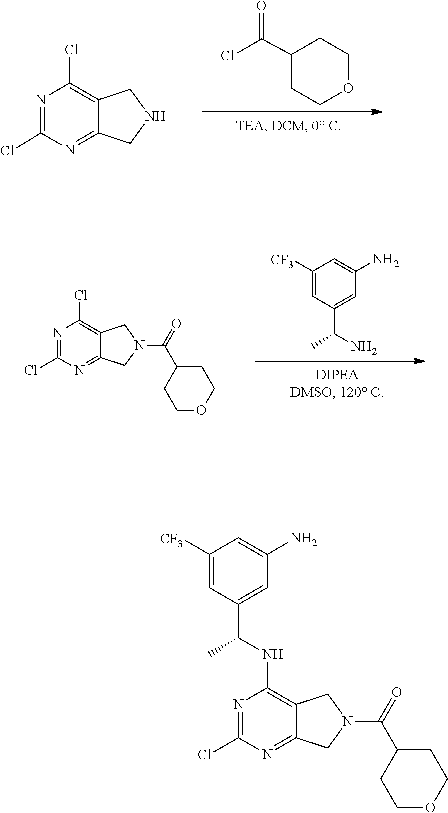 [SMILES: Clc1nc(Cl)c2c(n1)CNC2 ; O=C(Cl)C1CCOCC1 ; O=C(C1CCOCC1)N1Cc2nc(Cl)nc(Cl)c2C1 ; C[C@@H](N)c1cc(N)cc(C(F)(F)F)c1 ; C[C@@H](Nc1nc(Cl)nc2c1CN(C(=O)C1CCOCC1)C2)c1cc(N)cc(C(F)(F)F)c1]

### Step 1.

To a mixture of 2,4-dichloro-5H,6H,7H-pyrrolo[3,4-d]pyrimidine HCl salt (400 mg, 1.78 mmol) and TEA (990 μL, 7.13 mmol) in DCM (12 mL) at 0° C. under an atmosphere of Ar was added a solution of tetrahydro-2H-pyran-4-carbonyl chloride (230 μL, 1.87 mmol) in DCM (1.0 mL). The mixture was stirred at 0° C. for 30 min, then H₂O added and the mixture was extracted with DCM (×5). The combined organic layers were dried over anhydrous Na₂SO₄, filtered, then the solvent was concentrated under reduced pressure and the residue was purified by column chromatography to give 2,4-dichloro-6-(oxane-4-carbonyl)-5H,6H,7H-pyrrolo[3,4-d]pyrimidine (463 mg, 86% yield). LCMS (ESI): m/z: [M+H] calculated for C₁₂H₁₃Cl₂N₃O₂: 301.04. found 302.5; ¹H NMR (300 MHz, DMSO-d₆) δ 5.31-4.91 (m, 2H), 4.77-4.43 (m, 2H), 4.07-3.72 (m, 2H), 3.67-3.35 (m, 2H), 3.01-2.71 (m, 1H), 1.94-1.52 (m, 4H).

To an Ar-purged mixture of 2,4-dichloro-6-(oxane-4-carbonyl)-5H,6H,7H-pyrrolo[3,4-d]pyrimidine (200 mg, 0.66 mmol) and 3-[(1R)-1-aminoethyl]-5-(trifluoromethyl)aniline HCl salt (142 mg, 0.69 mmol) in DMSO (6.0 mL) was added DIPEA (460 μL, 2.6 mmol). The mixture was heated to 120° C. under microwave irradiation and stirred for 30 min, then diluted with Et₂O and washed with H₂O. The aqueous layer was extracted with Et₂O and the combined organic layers were washed with water (×2), dried over anhydrous Na₂SO₄, and filtered. The solvent was concentrated under reduced pressure and the residue was purified by prep-HPLC to give N-[(1R)-1-[3-amino-5-(trifluoromethyl)phenyl]ethyl]-2-chloro-6-(oxane-4-carbonyl)-5H,6H,7H-pyrrolo[3,4-d]pyrimidin-4-amine (180 mg). LCMS (ESI): m/z: [M+H] calculated for C₂₁H₂₃ClF₃N₅O₂: 469.15. found 470.0; ¹H NMR (400 MHz, DMSO-d₆) δ ppm 7.71 (s, 1H), 6.87 (d, J=4.7 Hz, 2H), 6.79 (s, 1H), 5.31-5.21 (m, 1H), 5.19-5.13 (m, 2H), 4.60 (s, 4H), 3.93 (d, J=11.4 Hz, 2H), 3.51-3.35 (m, 2H), 2.80-2.72 (m, 1H), 1.73-1.65 (m, 4H), 1.52 (d, J=7.0 Hz, 3H).

### Example 117. Synthesis of methyl 4-{[(1R)-1-[3-amino-5-(trifluoromethyl)phenyl]ethyl]amino}-2-methyl-5H,6H,7H-pyrrolo[3,4-d]pyrimidine-6-carboxylate

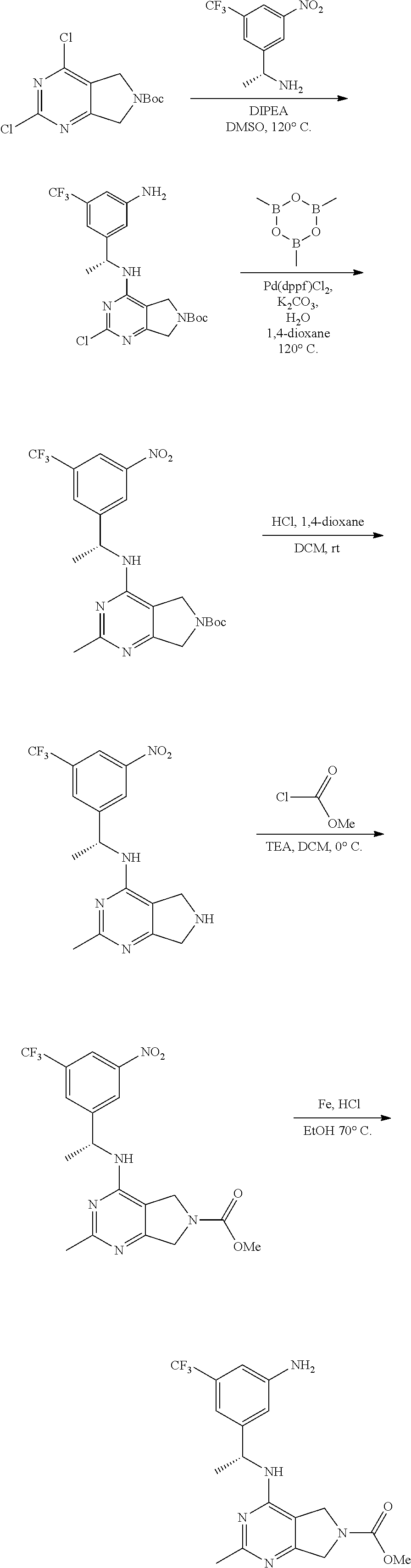

### Step 1.

Tert-butyl 2-chloro-4-{[(1R)-1-[3-nitro-5-(trifluoromethyl)phenyl]ethyl]amino}-5H,6H,7H-pyrrolo[3,4-d]pyrimidine-6-carboxylate was synthesized in a manner similar to 2-chloro-N-[(1R)-1-[3-nitro-5-(trifluoromethyl)phenyl]ethyl]-7-(oxolan-3-ylmethyl)-5H,6H,7H,8H,9H-pyrimido[4,5-d]azepin-4-amine except 2,4-dichloro-7-(oxolan-3-ylmethyl)-5H,6H,7H,8H,9H-pyrimido[4,5-d]azepine was substituted with tert-butyl 2,4-dichloro-5H,6H,7H-pyrrolo[3,4-d]pyrimidine-6-carboxylate. LCMS (ESI): m/z: [M+H] calculated for C₂₀H₂₁ClF₃N₅O₄: 487.12. found 487.95; ¹H NMR (300 MHz, DMSO-d₆) δ ppm 8.55 (s, 1H), 8.37 (s, 1H), 8.31 (d, J=7.7 Hz, 1H), 8.27 (s, 1H), 5.51-5.40 (m, 1H), 4.50-4.29 (m, 4H), 1.54 (d, J=7.0 Hz, 3H), 1.50-1.40 (m, 9H).

### Step 2.

To an Ar-purged mixture of tert-butyl 2-chloro-4-{[(1R)-1-[3-nitro-5-(trifluoromethyl)phenyl]ethyl]amino}-5H,6H,7H-pyrrolo[3,4-d]pyrimidine-6-carboxylate (795 mg, 1.63 mmol) and trimethylboroxine, 50% solution in THF (1.23 g, 9.8 mmol) in 1,4-dioxane (12.7 mL) was added K₂CO₃ (450 mg, 3.26 mmol) in H₂O (8 mL). The mixture was purged with Ar for another 15 min, then Pd(dppf)Cl₂ (119 mg, 0.16 mmol) was added. The mixture was heated to 120° C. under microwave irradiation and stirred for 1 h. Next, further trimethylboroxine, 50% solution in THF (409 mg, 3.26 mmol) and Pd(dppf)Cl₂ (60 mg, 81 μmol) were added, then the mixture heated to 120° C. under microwave irradiation and stirred for 45 min. The mixture was filtered through a pad of Celite® and the filter cake was washed with EtOAc. The filtrate was washed with H₂O, then dried over anhydrous Na₂SO₄ and filtered. The solvent was concentrated under reduced pressure and the residue was purified by column chromatography to give tert-butyl 2-methyl-4-{[(1R)-1-[3-nitro-5-(trifluoromethyl)phenyl]ethyl]amino}-5H,6H,7H-pyrrolo[3,4-d]pyrimidine-6-carboxylate (258 mg, 34% yield). LCMS (ESI): m/z: [M+H] calculated for C₂₁H₂₄F₃N₅O₄: 467.18. found 468.30; ¹H NMR (300 MHz, DMSO-d₆) δ ppm 8.56 (s, 1H), 8.33 (s, 1H), 8.27 (s, 1H), 7.74 (d, J=7.6 Hz, 1H), 5.60-5.43 (m, 1H), 4.55-4.24 (m, 4H), 2.27 (s, 3H), 1.53 (d, J=7.0 Hz, 3H), 1.50-1.40 (m, 9H).

### Step 3.

A mixture of tert-butyl 2-methyl-4-{[(1R)-1-[3-nitro-5-(trifluoromethyl)phenyl]ethyl]amino}-5H,6H,7H-pyrrolo[3,4-d]pyrimidine-6-carboxylate (233 mg, 0.50 mmol) in DCM (3.5 mL) and 4M HCl in 1,4-dioxane (1.6 mL, 6.5 mmol) was stirred at rt overnight. The solvent was concentrated under reduced pressure to give 2-methyl-N-[(1R)-1-[3-nitro-5-(trifluoromethyl)phenyl]ethyl]-5H,6H,7H-pyrrolo[3,4-d]pyrimidin-4-amine HCl salt (198 mg, 90% yield). LCMS (ESI): m/z: [M+H] calculated for C₁₆H₁₈Cl₂F₃N₅O₂: 367.13. found 367.90; ¹H NMR (300 MHz, METHANOL-d₄) δ ppm 8.58 (s, 1H), 8.46 (s, 1H), 8.21 (s, 1H), 5.77-5.68 (m, 1H), 4.69-4.61 (m, 4H), 2.57 (s, 3H), 1.73 (d, J=7.1 Hz, 3H).

### Step 4.

To a mixture of 2-methyl-N-[(1R)-1-[3-nitro-5-(trifluoromethyl)phenyl]ethyl]-5H,6H,7H-pyrrolo[3,4-d]pyrimidin-4-amine HCl salt (163 mg, 0.37 mmol) in DCM (4.9 mL) and TEA (258 μL, 1.85 mmol) at 0° C. under an atmosphere of Ar was added a solution of methyl chloroformate (66 mg, 0.37 mmol) in DCM (0.8 mL). The mixture was stirred at 0° C. for 1 h, then washed with saturated NH₄Cl (×2) and saturated NaHCO₃ (×1). The NH₄Cl aqueous layer was extracted with DCM (×3), and the NaHCO₃ aqueous layer was extracted with DCM (×2). The combined organic layers were dried over anhydrous Na₂SO₄, filtered and the solvent was concentrated under reduced pressure to give methyl 2-methyl-4-{[(1R)-1-[3-nitro-5-(trifluoromethyl)phenyl]ethyl]amino}-5H,6H,7H-pyrrolo[3,4-d]pyrimidine-6-carboxylate (146 mg, 93% yield). LCMS (ESI): m/z: [M+H] calculated for C₁₈H₁₈F₃N₅O₄: 425.13. found 426.00; ¹H NMR (300 MHz, DMSO-d₆) δ ppm 8.57 (s, 1H), 8.34 (s, 1H), 8.28 (s, 1H), 7.79-7.67 (m, 1H), 5.58-5.46 (m, 1H), 4.56-4.31 (m, 4H), 3.68 (d, J=2.9 Hz, 3H), 2.29 (s, 3H), 1.53 (d, J=7.0 Hz, 3H).

### Step 5.

Methyl 4-{[(1R)-1-[3-amino-5-(trifluoromethyl)phenyl]ethyl]amino}-2-methyl-5H,6H,7H-pyrrolo[3,4-d]pyrimidine-6-carboxylate was synthesized in a manner similar to N-[(1R)-1-[3-amino-5-(trifluoromethyl)phenyl]ethyl]-2-chloro-7-(oxolan-3-ylmethyl)-5H,6H,7H,8H,9H-pyrimido[4,5-d]azepin-4-amine except 2-chloro-N-[(1R)-1-[3-nitro-5-(trifluoromethyl)phenyl]ethyl]-7-(oxolan-3-ylmethyl)-5H,6H,7H,8H,9H-pyrimido[4,5-d]azepin-4-amine was substituted with methyl 2-methyl-4-{[(1R)-1-[3-nitro-5-(trifluoromethyl)phenyl]ethyl]amino}-5H,6H,7H-pyrrolo[3,4-d]pyrimidine-6-carboxylate. LCMS (ESI): m/z: [M+H] calculated for C₁₈H₂₀F₃N₅O₂: 395.16. found 396.18; ¹H NMR (300 MHz, DMSO-d₆) δ 7.68-7.38 (m, 1H), 6.84 (s, 1H), 6.78 (s, 1H), 6.68 (s, 1H), 5.53 (s, 2H), 5.39-5.22 (m, 1H), 4.46 (d, J=9.2 Hz, 2H), 4.37 (d, J=9.1 Hz, 2H), 3.67 (s, 3H), 2.31 (s, 3H), 1.42 (d, J=7.0 Hz, 3H).

### Example 118. Synthesis of oxan-4-yl 4-{[(1R)-1-[3-amino-5-(trifluoromethyl)phenyl]ethyl]amino}-2-methyl-5H,6H,7H-pyrrolo[3,4-d]pyrimidine-6-carboxylate

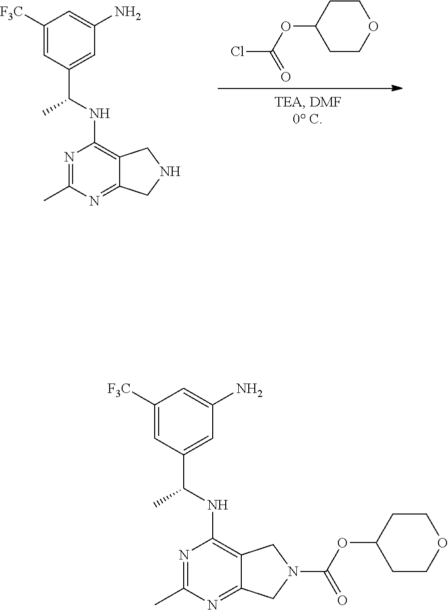 [SMILES: Cc1nc2c(c(N[C@H](C)c3cc(N)cc(C(F)(F)F)c3)n1)CNC2 ; O=C(Cl)OC1CCOCC1 ; Cc1nc2c(c(N[C@H](C)c3cc(N)cc(C(F)(F)F)c3)n1)CN(C(=O)OC1CCOCC1)C2]

### Step 1.

To a mixture of N-[(1R)-1-[3-amino-5-(trifluoromethyl)phenyl]ethyl]-2-methyl-5H,6H,7H-pyrrolo[3,4-d]pyrimidin-4-amine HCl salt (228 mg, 0.51 mmol) in DMF (6.8 mL) and TEA (355 μL, 2.55 mmol) at 0° C. under an atmosphere of Ar was added a solution of oxan-4-yl chloroformate (84 mg, 0.51 mmol) in DMF (2.3 mL). The mixture was stirred at 0° C. for 1 h, then a saturated solution of NH₄Cl was added. The mixture was extracted with Et₂O/EtOAc and the aqueous layer was extracted with Et₂O. The combined organic layers were washed with H₂O and brine, then dried over anhydrous Na₂SO₄ and filtered. The solvent was concentrated under reduced pressure and the residue was purified by prep-HPLC to give oxan-4-yl 4-{[(1R)-1-[3-amino-5-(trifluoromethyl)phenyl]ethyl]amino}-2-methyl-5H,6H,7H-pyrrolo[3,4-d]pyrimidine-6-carboxylate (88 mg, 37% yield). LCMS (ESI): m/z: [M+H] calculated for C₂₂H₂₆F₃N₅O₃: 465.20. found 466.13; ¹H NMR (400 MHz, DMSO-d₆) δ ppm 7.17 (d, J=8.0 Hz, 1H), 6.88 (s, 1H), 6.86 (s, 1H), 6.74 (s, 1H), 5.38-5.30 (m, 1H), 5.20 (s, 2H), 4.90-4.82 (m, 1H), 4.50 (s, 2H), 4.39 (s, 2H), 3.89-3.81 (m, 2H), 3.56-3.47 (m, 2H), 2.34 (s, 3H), 1.98-1.89 (m, 2H), 1.68-1.57 (m, 2H), 1.48 (d, J=7.0 Hz, 3H).

### Example 119. Synthesis of N-[(1R)-1-[3-amino-5-(trifluoromethyl)phenyl]ethyl]-2-methyl-6-(oxane-4-carbonyl)-5H,6H,7H-pyrrolo[3,4-d]pyrimidin-4-amine

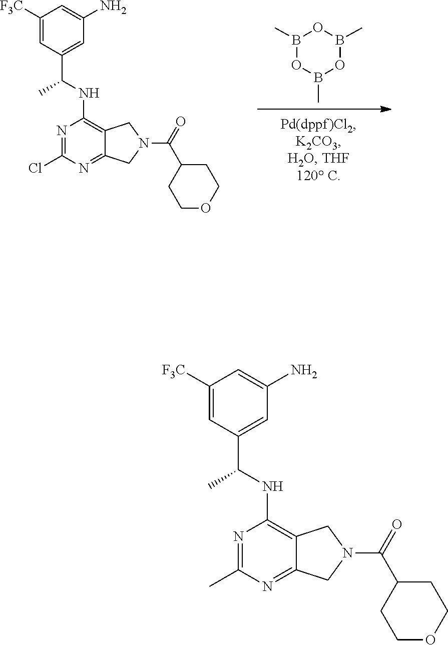

### Step 1.

To an Ar-purged mixture of N-[(1R)-1-[3-amino-5-(trifluoromethyl)phenyl]ethyl]-2-chloro-6-(oxane-4-carbonyl)-5H,6H,7H-pyrrolo[3,4-d]pyrimidin-4-amine (336 mg, 0.72 mmol) and trimethylboroxine, 50% solution in THF (539 mg, 4.29 mmol) was added K₂CO₃ (198 mg, 1.43 mmol) in H₂O (3.4 mL). The mixture was purged with Ar for a further 15 min, then Pd(dppf)Cl₂ (52 mg, 72 μmol) was added. The mixture was heated to 120° C. under microwave irradiation and stirred for 1 h. The mixture was filtered through a pad of Celite® and the filter cake was washed with EtOAc. The filtrate was washed with H₂O and brine, then dried over anhydrous Na₂SO₄ and filtered. The solvent was concentrated under reduced pressure and the residue was purified by prep-HPLC to give N-[(1R)-1-[3-amino-5-(trifluoromethyl)phenyl]ethyl]-2-methyl-6-(oxane-4-carbonyl)-5H,6H,7H-pyrrolo[3,4-d]pyrimidin-4-amine (49 mg). LCMS (ESI): m/z: [M+H] calculated for C₂₂H₂₆F₃N₅O₂: 449.20. found 450.00; ¹H NMR (400 MHz, DMSO-d₆) δ ppm 7.20 (s, 1H), 6.90-6.83 (m, 2H), 6.74 (s, 1H), 5.39-5.30 (m, 1H), 5.19 (s, 2H), 4.75-4.34 (m, 4H), 3.91 (s, 2H), 3.51-3.32 (m, 2H), 2.84-2.72 (m, 1H), 2.35 (s, 3H), 1.74-1.61 (m, 4H), 1.48 (d, J=7.0 Hz, 3H).

### Example 120. Synthesis of 1-(4-{[(1R)-1-[3-amino-5-(trifluoromethyl)phenyl]ethyl]amino}-2-methyl-5H,6H,7H-pyrrolo[3,4-d]pyrimidin-6-yl)ethan-1-one

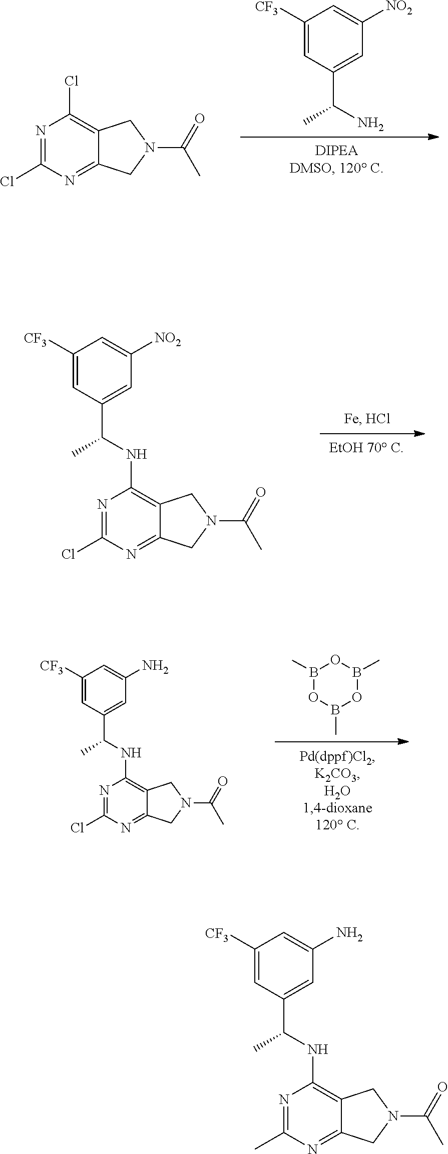

### Step 1.

1-(2-chloro-4-{[(1R)-1-[3-nitro-5-(trifluoromethyl)phenyl]ethyl]amino}-5H,6H,7H-pyrrolo[3,4-d]pyrimidin-6-yl)ethan-1-one was synthesized in a manner similar to 2-chloro-N-[(1R)-1-[3-nitro-5-(trifluoromethyl)phenyl]ethyl]-7-(oxolan-3-ylmethyl)-5H,6H,7H,8H,9H-pyrimido[4,5-d]azepin-4-amine except 2,4-dichloro-7-(oxolan-3-ylmethyl)-5H,6H,7H,8H,9H-pyrimido[4,5-d]azepine was substituted with 1-{2,4-dichloro-5H,6H,7H-pyrrolo[3,4-d]pyrimidin-6-yl}ethan-1-one. LCMS (ESI): m/z: [M+H] calculated for C₁₇H₁₅ClF₃N₅O₃: 429.08. found 429.95.

### Step 2.

To a mixture of 1-(2-chloro-4-{[(1R)-1-[3-nitro-5-(trifluoromethyl)phenyl]ethyl]amino}-5H,6H,7H-pyrrolo[3,4-d]pyrimidin-6-yl)ethan-1-one (583 mg, 1.36 mmol) in EtOH (12 mL) and 1M HCl (5.4 mL) was added Fe powder (417 mg, 7.46 mmol). The mixture was heated to 70° C. and stirred for 5 h. EtOAc was added and the mixture was washed with saturated NaHCO₃, then dried over anhydrous Na₂SO₄, filtered and the solvent concentrated under reduced pressure to give 1-(4-{[(1R)-1-[3-amino-5-(trifluoromethyl)phenyl]ethyl]amino}-2-chloro-5H,6H,7H-pyrrolo[3,4-d]pyrimidin-6-yl)ethan-1-one (443 mg). LCMS (ESI): m/z: [M+H] calculated for C₁₇H₁₇ClF₃N₅O: 399.11. found 399.90; ¹H NMR (300 MHz, DMSO-d₆) δ ppm 8.18 (d, J=8.0 Hz, 1H), 6.83 (s, 1H), 6.77 (d, J=5.8 Hz, 1H), 6.75-6.62 (m, 1H), 5.57 (s, 2H), 5.20 (q, J=7.0 Hz, 1H), 4.71-4.58 (m, 2H), 4.51-4.31 (m, 2H), 2.05 (d, J=9.7 Hz, 3H), 1.45 (dd, J=7.0, 5.4 Hz, 3H).

### Step 3.

1-(4-{[(1R)-1-[3-amino-5-(trifluoromethyl)phenyl]ethyl]amino}-2-methyl-5H,6H,7H-pyrrolo[3,4-d]pyrimidin-6-yl)ethan-1-one was synthesized in a manner similar to N-[(1R)-1-[3-amino-5-(trifluoromethyl)phenyl]ethyl]-2-methyl-6-(oxane-4-carbonyl)-5H,6H,7H-pyrrolo[3,4-d]pyrimidin-4-amine except N-[(1R)-1-[3-amino-5-(trifluoromethyl)phenyl]ethyl]-2-chloro-6-(oxane-4-carbonyl)-5H,6H,7H-pyrrolo[3,4-d]pyrimidin-4-amine was substituted with 1-(4-{[(1R)-1-[3-amino-5-(trifluoromethyl)phenyl]ethyl]amino}-2-chloro-5H,6H,7H-pyrrolo[3,4-d]pyrimidin-6-yl)ethan-1-one. LCMS (ESI): m/z: [M+H] calculated for C₁₈H₂₀F₃N₅O: 379.16. found 380.27; H NMR (400 MHz, DMSO-d₆, 100° C.) δ ppm 7.17 (s, 1H), 6.88 (d, J=7.1 Hz, 2H), 6.74 (s, 1H), 5.42-5.30 (m, 1H), 5.20 (s, 2H), 4.60 (d, J=20.9 Hz, 2H), 4.42 (d, J=42.1 Hz, 2H), 2.35 (s, 3H), 2.06 (s, 3H), 1.48 (d, J=6.8 Hz, 3H).

### Example 121. Synthesis of N-[(1R)-1-[3-amino-5-(trifluoromethyl)phenyl]ethyl]-2-chloro-6-(pyridine-3-carbonyl)-5H,6H,7H-pyrrolo[3,4-d]pyrimidin-4-amine

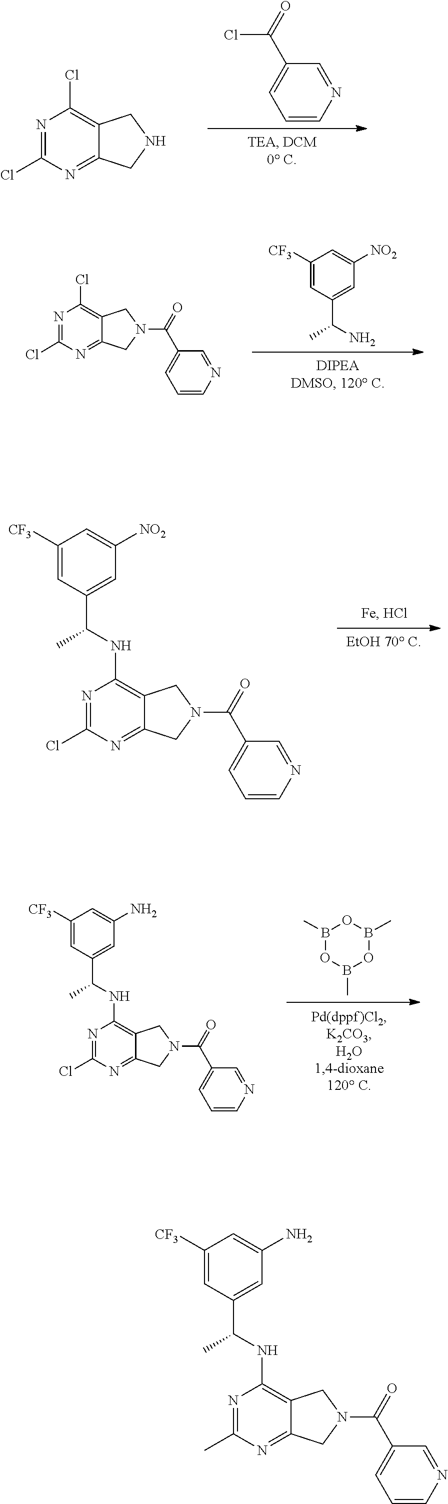

### Step 1.

3-{2,4-dichloro-5H,6H,7H-pyrrolo[3,4-d]pyrimidine-6-carbonyl}pyridine was synthesized in a manner similar to 2,4-dichloro-6-(oxane-4-carbonyl)-5H,6H,7H-pyrrolo[3,4-d]pyrimidine except tetrahydro-2H-pyran-4-carbonyl chloride was substituted with pyridine-3-carbonyl chloride. LCMS (ESI): m/z: [M+H] calculated for C₁₂H₈Cl₂N₄O: 294.01. found 294.90; ¹H NMR (300 MHz, DMSO-d₆) δ ppm 8.84 (d, J=2.2 Hz, 1H), 8.75 (dt, J=4.8, 2.3 Hz, 1H), 8.10 (dt, J=7.9, 2.0 Hz, 1H), 7.58 (dt, J=8.5, 4.6 Hz, 1H), 4.99 (d, J=6.9 Hz, 2H), 4.92 (d, J=3.7 Hz, 2H).

### Step 2.

2-Chloro-N-[(1R)-1-[3-nitro-5-(trifluoromethyl)phenyl]ethyl]-6-(pyridine-3-carbonyl)-5H,6H,7H-pyrrolo[3,4-d]pyrimidin-4-amine was synthesized in a manner similar to 2-chloro-N-[(1R)-1-[3-nitro-5-(trifluoromethyl)phenyl]ethyl]-7-(oxolan-3-ylmethyl)-5H,6H,7H,8H,9H-pyrimido[4,5-d]azepin-4-amine except 2,4-dichloro-7-(oxolan-3-ylmethyl)-5H,6H,7H,8H,9H-pyrimido[4,5-d]azepine was substituted with 3-{2,4-dichloro-5H,6H,7H-pyrrolo[3,4-d]pyrimidine-6-carbonyl}pyridine. LCMS (ESI): m/z: [M+H] calculated for C₂₁H₁₆ClF₃N₆O₃: 492.09. found 492.95; ¹H NMR (300 MHz, DMSO-d₆) δ ppm 8.83 (d, J=9.7 Hz, 1H), 8.77-8.66 (m, 1H), 8.59 (s, 1H), 8.50 (s, 1H), 8.45-8.33 (m, 1H), 8.27 (d, J=22.1 Hz, 1H), 8.16-7.92 (m, 1H), 7.64-7.42 (m, 1H), 5.64-5.37 (m, 1H), 4.98-4.35 (m, 4H), 1.74-1.43 (m, 3H).

### Step 3.

N-[(1R)-1-[3-amino-5-(trifluoromethyl)phenyl]ethyl]-2-chloro-6-(pyridine-3-carbonyl)-5H,6H,7H-pyrrolo[3,4-d]pyrimidin-4-amine was synthesized in a manner similar to 1-(4-{[(1R)-1-[3-amino-5-(trifluoromethyl)phenyl]ethyl]amino}-2-chloro-5H,6H,7H-pyrrolo[3,4-d]pyrimidin-6-yl)ethan-1-one except 1-(2-chloro-4-{[(1R)-1-[3-nitro-5-(trifluoromethyl)phenyl]ethyl]amino}-5H,6H,7H-pyrrolo[3,4-d]pyrimidin-6-yl)ethan-1-one was substituted with 2-chloro-N-[(1R)-1-[3-nitro-5-(trifluoromethyl)phenyl]ethyl]-6-(pyridine-3-carbonyl)-5H,6H,7H-pyrrolo[3,4-d]pyrimidin-4-amine. LCMS (ESI): m/z: [M+H] calculated for C₂₁H₁₈ClF₃N₆O: 462.86. found 463.0.

### Step 4.

N-[(1R)-1-[3-amino-5-(trifluoromethyl)phenyl]ethyl]-2-methyl-6-(pyridine-3-carbonyl)-5H,6H,7H-pyrrolo[3,4-d]pyrimidin-4-amine was synthesized in a manner similar to N-[(1R)-1-[3-amino-5-(trifluoromethyl)phenyl]ethyl]-2-methyl-6-(oxane-4-carbonyl)-5H,6H,7H-pyrrolo[3,4-d]pyrimidin-4-amine except N-[(1R)-1-[3-amino-5-(trifluoromethyl)phenyl]ethyl]-2-chloro-6-(oxane-4-carbonyl)-5H,6H,7H-pyrrolo[3,4-d]pyrimidin-4-amine was substituted with N-[(1R)-1-[3-amino-5-(trifluoromethyl)phenyl]ethyl]-2-chloro-6-(pyridine-3-carbonyl)-5H,6H,7H-pyrrolo[3,4-d]pyrimidin-4-amine. LCMS (ESI): m/z: [M+H] calculated for C₂₂H₂₁F₃N₆O: 442.17. found 443.17; ¹H NMR (400 MHz, DMSO-d₆, 100° C.) δ ppm 8.82 (d, J=2.3 Hz, 1H), 8.74-8.65 (m, 1H), 8.01 (dt, J=7.9, 2.0 Hz, 1H), 7.57-7.42 (m, 1H), 7.20 (s, 1H), 6.86 (s, 2H), 6.74 (s, 1H), 5.40-5.29 (m, 1H), 5.19 (s, 2H), 4.66 (s, 2H), 4.60 (s, 2H), 2.35 (s, 3H), 1.47 (d, J=7.0 Hz, 3H).

### Example 122. Synthesis of 4-{[(1R)-1-[3-amino-5-(trifluoromethyl)phenyl]ethyl]amino}-N,2-dimethyl-5H,6H,7H-pyrrolo[3,4-d]pyrimidine-6-carboxamide

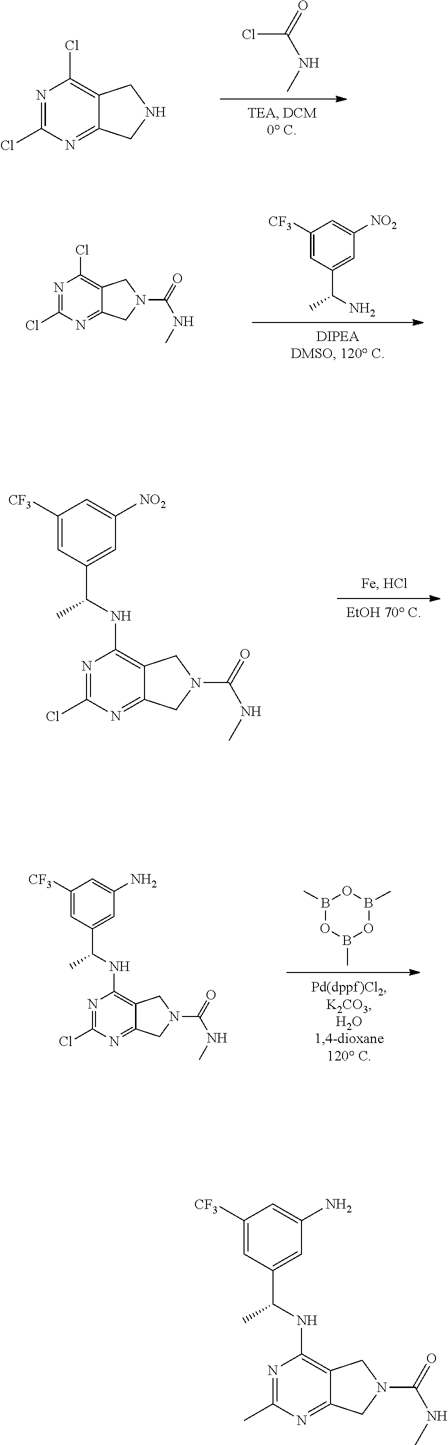

### Step 1.

2,4-dichloro-N-methyl-5H,6H,7H-pyrrolo[3,4-d]pyrimidine-6-carboxamide was synthesized in a manner similar to 2,4-dichloro-6-(oxane-4-carbonyl)-5H,6H,7H-pyrrolo[3,4-d]pyrimidine except tetrahydro-2H-pyran-4-carbonyl chloride was substituted with methylaminoformyl chloride. LCMS (ESI): m/z: [M+H] calculated for C₈H₈Cl₂N₄O: 246.01. found 247.4; ¹H NMR (300 MHz, DMSO-d₆) δ ppm 6.59-6.48 (m, 1H), 4.68-4.63 (m, 2H), 4.63-4.58 (m, 2H), 2.64 (d, J=4.3 Hz, 3H).

### Step 2.

2-Chloro-N-methyl-4-{[(1R)-1-[3-nitro-5-(trifluoromethyl)phenyl]ethyl]amino}-5H,6H,7H-pyrrolo[3,4-d]pyrimidine-6-carboxamide was synthesized in a manner similar to 2-chloro-N-[(1R)-1-[3-nitro-5-(trifluoromethyl)phenyl]ethyl]-7-(oxolan-3-ylmethyl)-5H,6H,7H,8H,9H-pyrimido[4,5-d]azepin-4-amine except 2,4-dichloro-7-(oxolan-3-ylmethyl)-5H,6H,7H,8H,9H-pyrimido[4,5-d]azepine was substituted with 2,4-dichloro-N-methyl-5H,6H,7H-pyrrolo[3,4-d]pyrimidine-6-carboxamide. LCMS (ESI): m/z: [M+H] calculated for C₁₇H₁₆ClF₃N₆O₃: 444.09. found 445.0; ¹H NMR (300 MHz, DMSO-d₆) δ ppm 8.57 (s, 1H), 8.37 (s, 1H), 8.29 (s, 1H), 8.25 (s, 1H), 6.32 (d, J=4.6 Hz, 1H), 5.53-5.39 (m, 1H), 4.45 (d, J=8.8 Hz, 2H), 4.36 (s, 2H), 2.64 (d, J=4.3 Hz, 3H), 1.54 (d, J=7.0 Hz, 3H).

### Step 3.

4-{[(1R)-1-[3-amino-5-(trifluoromethyl)phenyl]ethyl]amino}-2-chloro-N-methyl-5H,6H,7H-pyrrolo[3,4-d]pyrimidine-6-carboxamide was synthesized in a manner similar to 1-(4-{[(1R)-1-[3-amino-5-(trifluoromethyl)phenyl]ethyl]amino}-2-chloro-5H,6H,7H-pyrrolo[3,4-d]pyrimidin-6-yl)ethan-1-one except 1-(2-chloro-4-{[(1R)-1-[3-nitro-5-(trifluoromethyl)phenyl]ethyl]amino}-5H,6H,7H-pyrrolo[3,4-d]pyrimidin-6-yl)ethan-1-one was substituted with 2-chloro-N-methyl-4-{[(1R)-1-[3-nitro-5-(trifluoromethyl)phenyl]ethyl]amino}-5H,6H,7H-pyrrolo[3,4-d]pyrimidine-6-carboxamide. LCMS (ESI): m/z: [M+H] calculated for C₁₇H₁₈ClF₃N₆O: 414.12. found 415.0.

### Step 4.

4-{[(1R)-1-[3-amino-5-(trifluoromethyl)phenyl]ethyl]amino}-N,2-dimethyl-5H,6H,7H-pyrrolo[3,4-d]pyrimidine-6-carboxamide was synthesized in a manner similar to N-[(1R)-1-[3-amino-5-(trifluoromethyl)phenyl]ethyl]-2-methyl-6-(oxane-4-carbonyl)-5H,6H,7H-pyrrolo[3,4-d]pyrimidin-4-amine except N-[(1R)-1-[3-amino-5-(trifluoromethyl)phenyl]ethyl]-2-chloro-6-(oxane-4-carbonyl)-5H,6H,7H-pyrrolo[3,4-d]pyrimidin-4-amine was substituted with 4-{[(1R)-1-[3-amino-5-(trifluoromethyl)phenyl]ethyl]amino}-2-chloro-N-methyl-5H,6H,7H-pyrrolo[3,4-d]pyrimidine-6-carboxamide. LCMS (ESI): m/z: [M+H] calculated for C₁₈H₂₁F₃N₆O: 394.17. found 395.28; H NMR (300 MHz, DMSO-d₆) δ ppm 7.51 (d, J=8.2 Hz, 1H), 6.85 (s, 1H), 6.78 (s, 1H), 6.68 (s, 1H), 6.27 (d, J=4.7 Hz, 1H), 5.53 (s, 2H), 5.38-5.22 (m, 1H), 4.40 (s, 2H), 4.31 (s, 2H), 2.63 (d, J=4.1 Hz, 3H), 2.31 (s, 3H), 1.42 (d, J=7.0 Hz, 3H).

### Example 123. Synthesis of 4-{[(1R)-1-[3-amino-5-(trifluoromethyl)phenyl]ethyl]amino}-6-(morpholine-4-carbonyl)-5H,6H,7H-pyrrolo[3,4-d]pyrimidine-2-carbonitrile

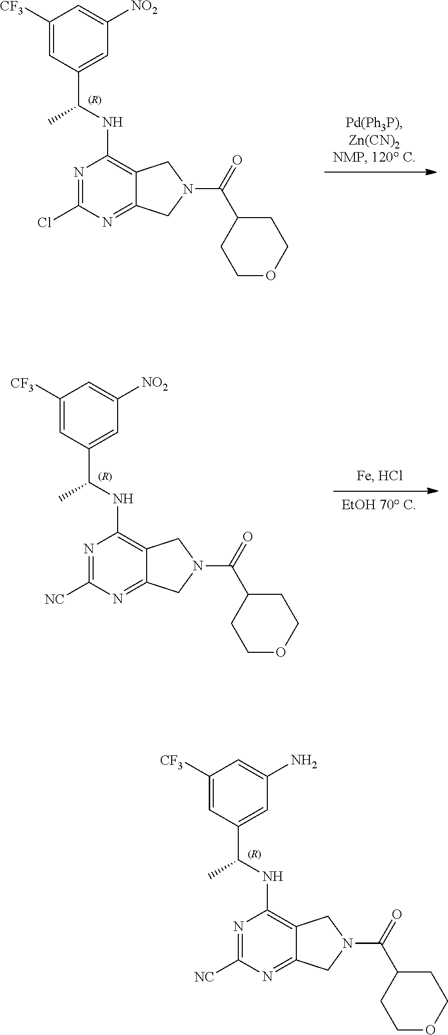

### Step 1.

A mixture of 2-chloro-6-(morpholine-4-carbonyl)-N-[(1R)-1-[3-nitro-5-(trifluoromethyl)phenyl]ethyl]-5H,6H,7H-pyrrolo[3,4-d]pyrimidin-4-amine (300 mg, 0.6 mmol), Zn(CN)₂ (84 mg, 0.72 mmol) and Pd(PPh₃)₄(48.5 mg, 42 μmol) in NMP (9 mL) under an atmosphere of Ar was heated to 120° C. and stirred overnight. A further aliquot of Zn(CN)₂ (84 mg, 0.72 mmol) and Pd(PPh₃)₄(48.5 mg, 42 μmol) were added and the mixture was stirred at 120° C. over a weekend. 2M NH₃ (12 mL) was added and the mixture was extracted with Et₂O (9×50 mL). The combined organic layers were dried over anhydrous Na₂SO₄, filtered, and the solvent concentrated under reduced pressure to give 6-(morpholine-4-carbonyl)-4-{[(1R)-1-[3-nitro-5-(trifluoromethyl)phenyl]ethyl]amino}-5H,6H,7H-pyrrolo[3,4-d]pyrimidine-2-carbonitrile (480 mg, >100% yield; note: crude product). LCMS (ESI): m/z: [M+H] calculated for C₂₁H₂₀F₃N₇O₄: 491.15. found 492.66.

### Step 2.

4-{[(1R)-1-[3-amino-5-(trifluoromethyl)phenyl]ethyl]amino}-6-(morpholine-4-carbonyl)-5H,6H,7H-pyrrolo[3,4-d]pyrimidine-2-carbonitrile was synthesized in a manner similar to N-[(1R)-1-[3-amino-5-(trifluoromethyl)phenyl]ethyl]-2-chloro-7-(oxolan-3-ylmethyl)-5H,6H,7H,8H,9H-pyrimido[4,5-d]azepin-4-amine except 2-chloro-N-[(1R)-1-[3-nitro-5-(trifluoromethyl)phenyl]ethyl]-7-(oxolan-3-ylmethyl)-5H,6H,7H,8H,9H-pyrimido[4,5-d]azepin-4-amine was substituted with 6-(morpholine-4-carbonyl)-4-{[(1R)-1-[3-nitro-5-(trifluoromethyl)phenyl]ethyl]amino}-5H,6H,7H-pyrrolo[3,4-d]pyrimidine-2-carbonitrile. LCMS (ESI): m/z: [M+H] calculated for C₂₁H₂₂F₃N₇O₂: 461.18. found 462.00; ¹H NMR (300 MHz, DMSO-d₆) δ ppm 8.28 (d, J=7.0 Hz, 1H), 6.84 (s, 1H), 6.78 (s, 1H), 6.71 (s, 1H), 5.72-5.51 (m, 2H), 5.30-5.18 (m, 1H), 4.78-4.49 (m, 4H), 3.63 (t, J=4.7 Hz, 4H), 3.26 (t, J=4.9 Hz, 4H), 1.46 (d, J=7.0 Hz, 3H).

### Example 124. Synthesis of N-[(1R)-1-[3-amino-5-(trifluoromethyl)phenyl]ethyl]-2-ethoxy-6-(morpholine-4-carbonyl)-5H,6H,7H-pyrrolo[3,4-d]pyrimidin-4-amine

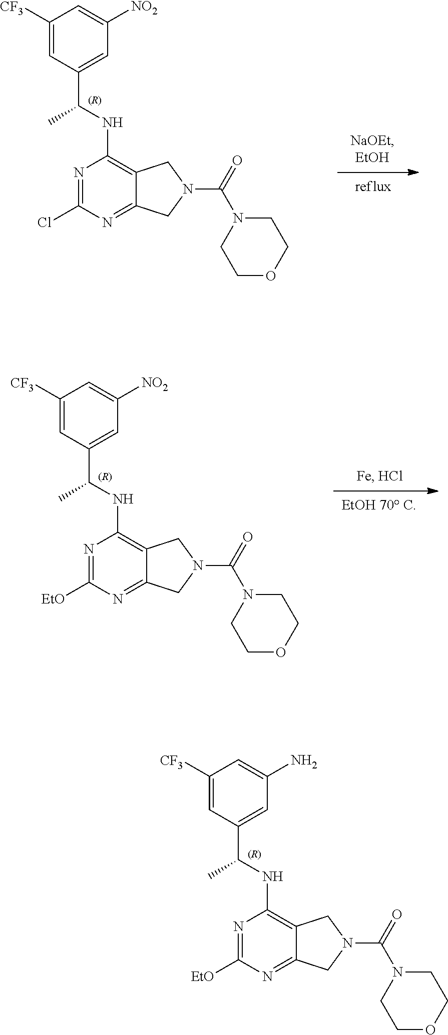

### Step 1.

A mixture of 2-chloro-6-(morpholine-4-carbonyl)-N-[(1R)-1-[3-nitro-5-(trifluoromethyl)phenyl]-ethyl]-5H,6H,7H-pyrrolo[3,4-d]pyrimidin-4-amine (400 mg, 0.8 mmol) and NaOEt (434 mg, 6.38 mmol) in EtOH (13.4 mL) was heated to reflux and stirred overnight. The solvent was concentrated under reduced pressure, H₂O was added, and the mixture extracted with EtOAc (×3). The combined organic layers were dried over anhydrous Na₂SO₄, filtered, concentrated under reduced pressure and the residue was purified by column chromatography to give 2-ethoxy-6-(morpholine-4-carbonyl)-N-[(1R)-1-[3-nitro-5-(trifluoromethyl)phenyl]-ethyl]-5H,6H,7H-pyrrolo[3,4-d]pyrimidin-4-amine (81 mg, 20% yield). LCMS (ESI): m/z: [M+H] calculated for C₂₂H₂₅F₃N₆O₅: 510.18. found 511.30; ¹H NMR (400 MHz, CDCl₃) δ ppm 8.41 (s, 1H), 8.39 (s, 1H), 7.96 (s, 1H), 5.43 (p, J=6.9 Hz, 1H), 4.77 (d, J=6.2 Hz, 1H), 4.64 (d, J=2.1 Hz, 2H), 4.53 (s, 2H), 4.21 (dd, J=10.5, 7.1 Hz, 1H), 4.08 (dq, J=10.6, 7.0 Hz, 1H), 3.76-3.70 (m, 4H), 3.43-3.30 (m, 4H), 1.66 (d, J=7.0 Hz, 3H), 1.27 (t, J=7.2 Hz, 3H).

### Step 2.

N-[(1R)-1-[3-amino-5-(trifluoromethyl)phenyl]ethyl]-2-ethoxy-6-(morpholine-4-carbonyl)-5H,6H,7H-pyrrolo[3,4-d]pyrimidin-4-amine was synthesized in a manner similar to N-[(1R)-1-[3-amino-5-(trifluoromethyl)phenyl]ethyl]-2-chloro-7-(oxolan-3-ylmethyl)-5H,6H,7H,8H,9H-pyrimido[4,5-d]azepin-4-amine except 2-chloro-N-[(1R)-1-[3-nitro-5-(trifluoromethyl)phenyl]ethyl]-7-(oxolan-3-ylmethyl)-5H,6H,7H,8H,9H-pyrimido[4,5-d]azepin-4-amine was substituted with 2-ethoxy-6-(morpholine-4-carbonyl)-N-[(1R)-1-[3-nitro-5-(trifluoromethyl)phenyl]-ethyl]-5H,6H,7H-pyrrolo[3,4-d]pyrimidin-4-amine. LCMS (ESI): m/z: [M+H] calculated for C22H27F₃N6O₃: 480.21. found 481.00; H NMR (300 MHz, DMSO-d₆) δ ppm 7.67 (d, J=7.4 Hz, 1H), 6.79 (s, 1H), 6.75 (s, 1H), 6.67 (d, J=2.5 Hz, 1H), 5.53 (s, 2H), 5.13 (t, J=7.1 Hz, 1H), 4.58-4.49 (m, 2H), 4.42 (s, 2H), 4.13 (q, J=7.0 Hz, 2H), 3.62 (t, J=4.6 Hz, 4H), 3.23 (t, J=4.6 Hz, 4H), 1.42 (d, J=7.0 Hz, 3H), 1.16 (t, J=7.0 Hz, 3H).

### Example 125. Synthesis of N4-[(1R)-1-[3-amino-5-(trifluoro-methyl)phenyl]ethyl]-N2-methyl-6-(morpholine-4-carbonyl)-5H,6H,7H-pyrrolo[3,4-d]pyrimidine-2,4-diamine

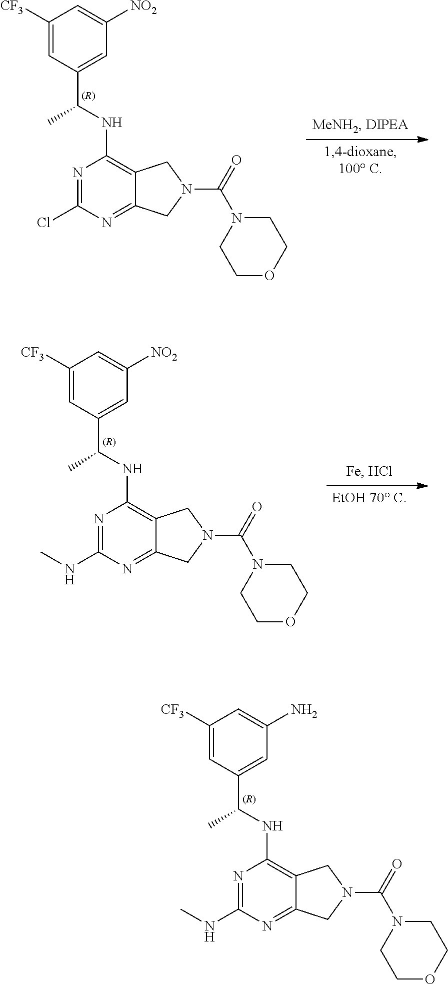

### Step 1.

To a mixture of 2-chloro-6-(morpholine-4-carbonyl)-N-[(1R)-1-[3-nitro-5-(trifluoromethyl)phenyl]-ethyl]-5H,6H,7H-pyrrolo[3,4-d]pyrimidin-4-amine (200 mg, 0.4 mmol) in 1.4-dioxane (2.6 mL) was added MeNH₂ (3.19 mL, 6.39 mmol) and DIPEA (0.28 mL, 1.6 mmol). The mixture was heated to 100° C. and stirred for 24 h, then the solvent was concentrated under reduced pressure, H₂O was added, and the mixture extracted with EtOAc. The combined organic layers were dried over anhydrous Na₂SO₄, filtered, and concentrated under reduced pressure to give N2-methyl-6-(morpholine-4-carbonyl)-N4-[(1R)-1-[3-nitro-5-(trifluoromethyl)phenyl]ethyl]-5H,6H,7H-pyrrolo[3,4-d]pyrimidine-2,4-diamine (180 mg). LCMS (ESI): m/z: [M+H] calculated for C₂₁H₂₄F₃N₇O₄: 495.18. found 496.00; ¹H NMR (300 MHz, DMSO-d₆) δ ppm 8.53 (s, 1H), 8.33 (s, 1H), 8.25 (s, 1H), 7.41 (d, J=7.5 Hz, 1H), 6.38 (d, J=4.9 Hz, 1H), 5.42 (q, J=7.2 Hz, 1H), 4.56-4.40 (m, 2H), 4.31 (s, 2H), 3.63 (t, J=4.7 Hz, 4H), 3.23 (t, J=4.8 Hz, 4H), 2.62 (d, J=4.7 Hz, 3H), 1.52 (d, J=7.1 Hz, 3H).

### Step 2.

N4-[(1R)-1-[3-amino-5-(trifluoro-methyl)phenyl]ethyl]-N2-methyl-6-(morpholine-4-carbonyl)-5H,6H,7H-pyrrolo[3,4-d]pyrimidine-2,4-diamine was synthesized in a manner similar to N-[(1R)-1-[3-amino-5-(trifluoromethyl)phenyl]ethyl]-2-chloro-7-(oxolan-3-ylmethyl)-5H,6H,7H,8H,9H-pyrimido[4,5-d]azepin-4-amine except 2-chloro-N-[(1R)-1-[3-nitro-5-(trifluoromethyl)phenyl]ethyl]-7-(oxolan-3-ylmethyl)-5H,6H,7H,8H,9H-pyrimido[4,5-d]azepin-4-amine was substituted with N2-methyl-6-(morpholine-4-carbonyl)-N4-[(1R)-1-[3-nitro-5-(trifluoromethyl)phenyl]ethyl]-5H,6H,7H-pyrrolo[3,4-d]pyrimidine-2,4-diamine. LCMS (ESI): m/z: [M+H] calculated for C₂₁H₂₆F₃N₇O₂: 465.21. found 465.86; ¹H NMR (300 MHz, DMSO-d₆) δ ppm 7.18 (d, J=7.8 Hz, 1H), 6.79 (d, J=9.7 Hz, 2H), 6.67 (d, J=1.9 Hz, 1H), 6.40-6.28 (m, 1H), 5.51 (s, 2H), 5.21 (t, J=6.8 Hz, 1H), 4.44 (d, J=6.0 Hz, 2H), 4.32 (s, 2H), 3.63 (t, J=4.5 Hz, 4H), 3.22 (t, J=4.6 Hz, 4H), 2.66 (d, J=4.7 Hz, 3H), 1.41 (d, J=7.0 Hz, 3H).

### Example 126. Synthesis of N-[(1R)-1-[3-amino-5-(trifluoro-methyl)phenyl]ethyl]-6-(morpholine-4-carbonyl)-2-(trifluoro-methoxy)-5H,6H,7H-pyrrolo-[3,4-d]pyrimidin-4-amine

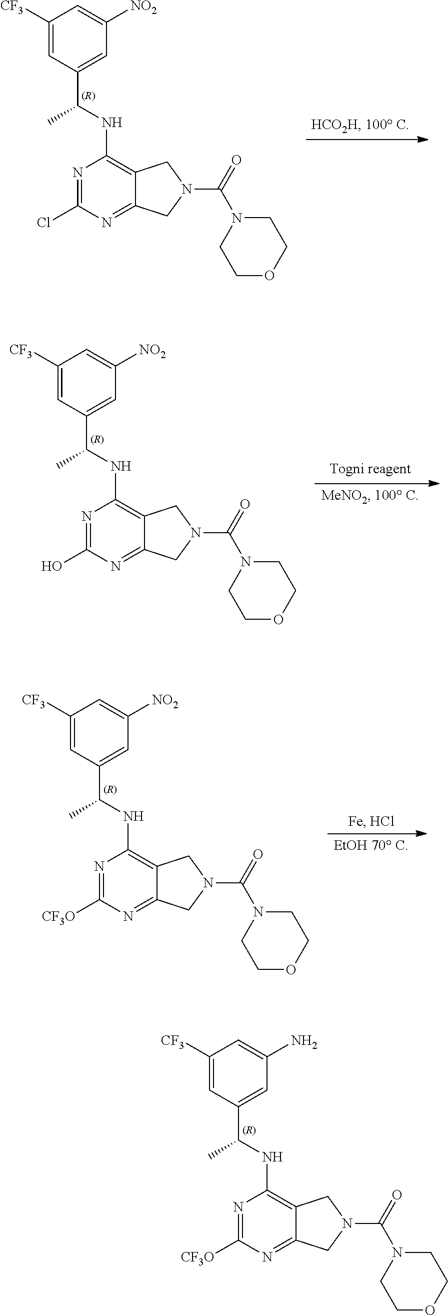

### Step 1.

A mixture of 2-chloro-6-(morpholine-4-carbonyl)-N-[(1R)-1-[3-nitro-5-(trifluoromethyl)phenyl]ethyl]-5H,6H,7H-pyrrolo-[3,4-d]pyrimidin-4-amine (500 mg, 1.0 mmol) in formic acid (12.5 mL) was stirred at 100° C. overnight. H₂O was added and the mixture extracted with DCM (×3). The combined organic layers were dried over anhydrous Na₂SO₄, filtered and the solvent concentrated under reduced pressure. The crude residue was triturated with DCM, filtered and the filter cake was washed with DCM to give 6-(morpholine-4-carbonyl)-4-{[(1R)-1-[3-nitro-5-(trifluoromethyl)phenyl]ethyl]-amino}-5H,6H,7H-pyrrolo[3,4-d]pyrimidin-2-ol (312 mg, 65% yield). LCMS (ESI): m/z: [M+H] calculated for C₂₀H₂₁F₃N₆O₅: 482.15. found 483.30; ¹H NMR (300 MHz, DMSO-d₆) δ ppm 10.86 (s, 1H), 8.51 (s, 1H), 8.36 (s, 1H), 8.24 (s, 1H), 7.90 (d, J=7.7 Hz, 1H), 5.51 (t, J=7.2 Hz, 1H), 4.66-4.20 (m, 4H), 3.61 (t, J=4.6 Hz, 4H), 3.23 (t, J=4.7 Hz, 4H), 1.50 (d, J=7.0 Hz, 3H).

### Step 2.

To a mixture of air-purged 6-(morpholine-4-carbonyl)-4-{[(1R)-1-[3-nitro-5-(trifluoromethyl)phenyl]ethyl]amino}-5H,6H,7H-pyrrolo-[3,4-d]-pyrimidin-2-ol (290 mg, 0.6 mmol) in MeNO₂ (6 mL) was added Togni reagent (298 mg, 0.9 mmol). The mixture was heated to 100° C. and stirred overnight. A further aliquot of Togni reagent (99 mg, 0.3 mmol) was added and the mixture was stirred at 100° C. for 4 h, then more Togni reagent (99 mg, 0.3 mmol) added and the mixture stirred for another 4 h. H₂O was added and the mixture was extracted with DCM (×3). The combined organic layers were dried over anhydrous Na₂SO₄, filtered, concentrated under reduced pressure and the residue was purified by column chromatography to give 6-(morpholine-4-carbonyl)-N-[(1R)-1-[3-nitro-5-(trifluoromethyl)-phenyl]ethyl]-2-(trifluoromethoxy)-5H,6H,7H-pyrrolo[3,4-d]pyrimidin-4-amine (130 mg, 39% yield). LCMS (ESI): m/z: [M+H] calculated for C₂₁H₂₀F₆N₆O₅: 550.14. found 551.05; ¹H NMR (400 MHz, DMSO-d₆) δ ppm 8.51 (s, 1H), 8.42 (d, J=7.0 Hz, 1H), 8.36 (s, 1H), 8.21 (s, 1H), 5.32 (t, J=7.0 Hz, 1H), 4.76-4.55 (m, 2H), 4.52 (s, 2H), 3.62 (t, J=4.7 Hz, 4H), 3.24 (t, J=4.6 Hz, 4H), 1.56 (d, J=7.1 Hz, 3H); ¹⁹F NMR (376 MHz, DMSO-dl₆) δ ppm −55.6, −61.4.

### Step 3.

N-[(1R)-1-[3-amino-5-(trifluoro-methyl)phenyl]ethyl]-6-(morpholine-4-carbonyl)-2-(trifluoro-methoxy)-5H,6H,7H-pyrrolo-[3,4-d]pyrimidin-4-amine was synthesized in a manner similar to N-[(1R)-1-[3-amino-5-(trifluoromethyl)phenyl]ethyl]-2-chloro-7-(oxolan-3-ylmethyl)-5H,6H,7H,8H,9H-pyrimido[4,5-d]azepin-4-amine except 2-chloro-N-[(1R)-1-[3-nitro-5-(trifluoromethyl)phenyl]ethyl]-7-(oxolan-3-ylmethyl)-5H,6H,7H,8H,9H-pyrimido[4,5-d]azepin-4-amine was substituted with 6-(morpholine-4-carbonyl)-N-[(1R)-1-[3-nitro-5-(trifluoromethyl)-phenyl]ethyl]-2-(trifluoromethoxy)-5H,6H,7H-pyrrolo[3,4-d]pyrimidin-4-amine. LCMS (ESI): m/z: [M+H] calculated for C₂₁H₂₂F₆N₆O₃: 520.17. found 521.17; ¹H NMR (300 MHz, DMSO-d₆) δ ppm 8.25 (d, J=7.6 Hz, 1H), 6.80 (s, 1H), 6.75 (s, 1H), 6.70 (s, 1H), 5.54 (s, 2H), 5.10 (t, J=7.0 Hz, 1H), 4.61-4.27 (m, 4H), 3.62 (t, J=4.6 Hz, 4H), 3.24 (t, J=4.7 Hz, 4H), 1.46 (d, J=7.0 Hz, 3H).

### Example 127. Synthesis of N-[(1R)-1-[3-amino-5-(trifluoromethyl)phenyl]ethyl]-6-(morpholine-4-carbonyl)-2-(trifluoromethyl)-5H,6H,7H-pyrrolo[3,4-d]pyrimidin-4-amine

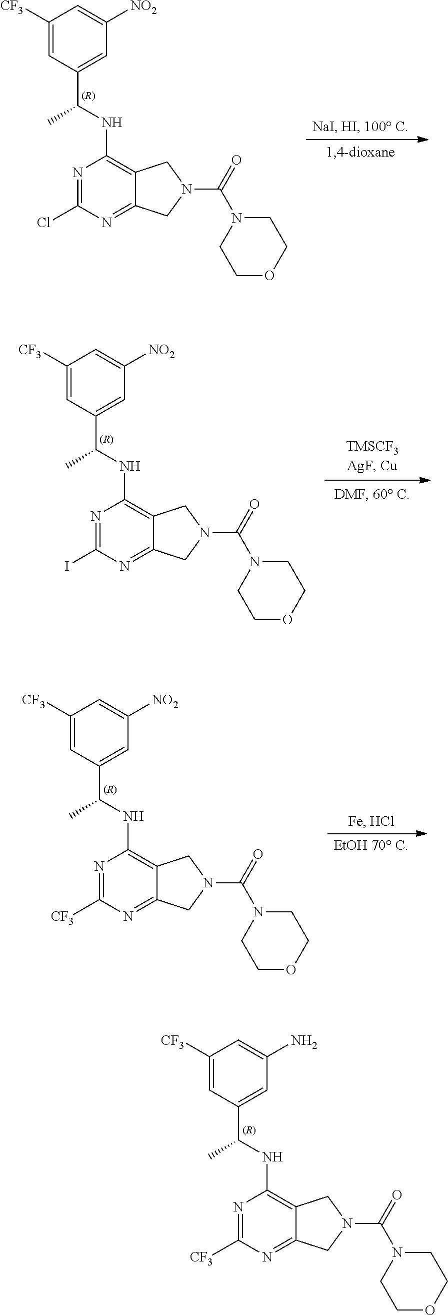

To a mixture of 2-chloro-6-(morpholine-4-carbonyl)-N-[(1R)-1-[3-nitro-5-(trifluoromethyl)phenyl]ethyl]-5H,6H,7H-pyrrolo[3,4-d]pyrimidin-4-amine (300 mg, 0.6 mmol) and NaI (359 mg, 2.4 mmol) in 1,4-dioxane (6 mL) was added HI, 57% aqueous solution (96 μL, 0.72 mmol). The mixture was heated to 100° C. and stirred overnight. Saturated aqueous Na₂CO₃ was added and the mixture was extracted with DCM (8×50 mL). The combined organic layers were dried over anhydrous Na₂SO₄, filtered and the solvent concentrated under reduced pressure to give 2-iodo-6-(morpholine-4-carbonyl)-N-[(1R)-1-[3-nitro-5-(trifluoromethyl)phenyl]ethyl]-5H,6H,7H-pyrrolo[3,4-d]pyrimidin-4-amine and 2-chloro-6-(morpholine-4-carbonyl)-N-[(1R)-1-[3-nitro-5-(trifluoromethyl)phenyl]ethyl]-5H,6H,7H-pyrrolo[3,4-d]pyrimidin-4-amine (approx. 4:6 ratio, 385 mg). The crude product was used to the next step without further purification. LCMS (ESI): m/z: [M+H] calculated for C₂₀H₂₀F₃IN₆O₄: 592.05. found 593.5.

### Step 2.

This reaction was undertaken in 4-batches in parallel on the scale illustrated below.

To a mixture of AgF (22.4 mg, 0.18 mmol) and CF₃SiMe₃ (0.12 mL, 0.8 mmol) stirred in DMF (1.9 mL) under an atmosphere of Ar for 20 min was added Cu (16.8 mg, 0.26 mmol). The mixture was stirred at rt for 2 h, then a mixture of 2-iodo-6-(morpholine-4-carbonyl)-N-[(1R)-1-[3-nitro-5-(trifluoromethyl)phenyl]ethyl]-5H,6H,7H-pyrrolo[3,4-d]pyrimidin-4-amine and 2-chloro-6-(morpholine-4-carbonyl)-N-[(1R)-1-[3-nitro-5-(trifluoromethyl)phenyl]ethyl]-5H,6H,7H-pyrrolo[3,4-d]pyrimidin-4-amine (4:6 ratio; 95 mg, 0.16 mmol) was added. The reaction mixture was heated to 60° C. and stirred overnight. The combined reaction mixtures were extracted with Et₂O (8×40 mL), the combined organic layers were dried over anhydrous Na₂SO₄ and filtered. The solvent was concentrated under reduced pressure to give 6-(morpholine-4-carbonyl)-N-[(1R)-1-[3-nitro-5-(trifluoro-methyl)phenyl]ethyl]-2-(trifluoromethyl)-5H,6H,7H-pyrrolo[3,4-d]pyrimidin-4-amine (210 mg, 61%). The crude product was used to the next step without further purification. LCMS (ESI): m/z: [M+H] calculated for C₂₁H₂₀F₆N₆O₄: 534.14. found 535.05.

### Step 3.

N-[(1R)-1-[3-amino-5-(trifluoromethyl)phenyl]ethyl]-6-(morpholine-4-carbonyl)-2-(trifluoromethyl)-5H,6H,7H-pyrrolo[3,4-d]pyrimidin-4-amine was synthesized in a manner similar to N-[(1R)-1-[3-amino-5-(trifluoromethyl)phenyl]ethyl]-2-chloro-7-(oxolan-3-ylmethyl)-5H,6H,7H,8H,9H-pyrimido[4,5-d]azepin-4-amine except 2-chloro-N-[(1R)-1-[3-nitro-5-(trifluoromethyl)phenyl]ethyl]-7-(oxolan-3-ylmethyl)-5H,6H,7H,8H,9H-pyrimido[4,5-d]azepin-4-amine was substituted with 6-(morpholine-4-carbonyl)-N-[(1R)-1-[3-nitro-5-(trifluoro-methyl)phenyl]ethyl]-2-(trifluoromethyl)-5H,6H,7H-pyrrolo[3,4-d]pyrimidin-4-amine. LCMS (ESI): m/z: [M+H] calculated for C₂₁H₂₂F₆N₆O₂: 504.17. found 505.18. ¹H NMR (300 MHz, DMSO-d₆) δ ppm 8.24 (d, J=7.8 Hz, 1H), 6.86 (s, 1H), 6.78 (s, 1H), 6.70 (s, 1H), 5.56 (s, 2H), 5.34-5.13 (m, 1H), 4.62 (s, 4H), 3.63 (t, J=5.2 Hz, 4H), 3.26 (t, J=4.9 Hz, 4H), 1.48 (d, J=7.0 Hz, 3H).

### Example 128. Synthesis of N-[(1R)-1-[3-amino-5-(trifluoromethyl)phenyl]ethyl]-2-ethyl-6-(morpholine-4-carbonyl)-5H,6H,7H-pyrrolo[3,4-d]pyrimidin-4-amine

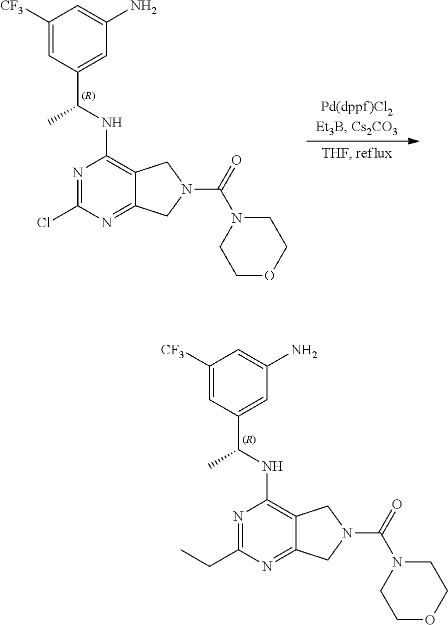

### Step 1.

To a mixture of N-[(1R)-1-[3-amino-5-(trifluoromethyl)phenyl]ethyl]-2-chloro-6-(morpholine-4-carbonyl)-5H,6H,7H-pyrrolo[3,4-d]pyrimidin-4-amine (250 mg, 0.53 mmol), Cs₂CO₃ (519 mg, 1.59 mmol) and Pd(dppf)Cl₂—CH₂Cl₂ (26 mg, 0.03 mmol) in THF (1 mL) under an atmosphere of Ar was added triethylborane, 1M solution in hexane (1.06 mL, 1.06 mmol). The mixture was heated to reflux and stirred overnight, then 1M HCl was added and the mixture stirred at rt for 1 h. NaHCO₃ was added, the mixture was extracted with DCM and the combined organic layers were dried over anhydrous Na₂SO₄ and filtered. The solvent was concentrated under reduced pressure and the residue was purified by prep-HPLC to give N-[(1R)-1-[3-amino-5-(trifluoromethyl)phenyl]ethyl]-2-ethyl-6-(morpholine-4-carbonyl)-5H,6H,7H-pyrrolo[3,4-d]pyrimidin-4-amine (66 mg, 27% yield). LCMS (ESI): m/z: [M+H] calculated for C₂₂H₂₇F₃N₆O₂: 464.21. found 465.15; ¹H NMR (300 MHz, DMSO-d₆) δ ppm 7.51 (d, J=7.7 Hz, 1H), 6.84 (s, 1H), 6.79 (s, 1H), 6.67 (s, 1H), 5.52 (s, 2H), 5.29 (q, J=7.6 Hz, 1H), 4.59-4.50 (m, 2H), 4.47 (s, 2H), 3.73-3.54 (m, 4H), 3.24 (t, J=4.7 Hz, 4H), 2.63-2.54 (m, 2H), 1.44 (d, J=7.0 Hz, 3H), 1.11 (t, J=7.6 Hz, 3H).

### Example 129. Synthesis of (4-{1[(1R)-1-[3-amino-5-(trifluoromethyl)-phenyl]ethyl]amino}-6-(morpholine-4-carbonyl)-5H,6H,7H-pyrrolo[3,4-d]pyrimidin-2-yl)methanol

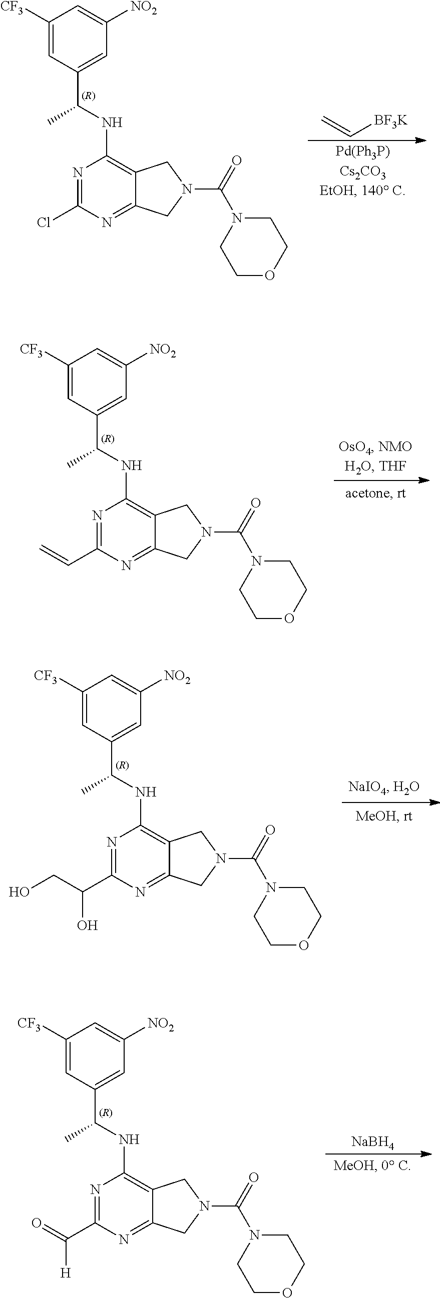

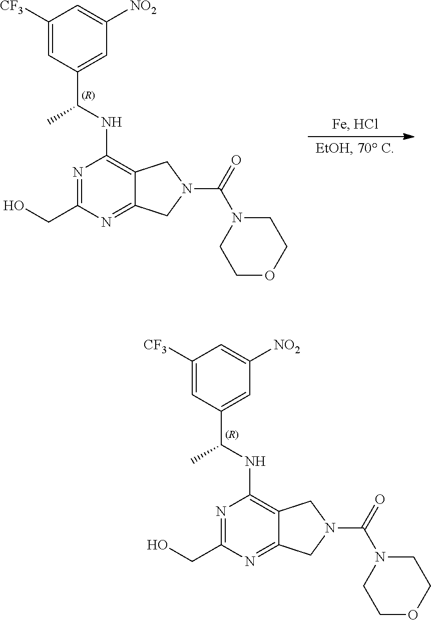

### Step 1.

To an Ar-purged mixture of 2-chloro-6-(morpholine-4-carbonyl)-N-[(1R)-1-[3-nitro-5-(trifluoromethyl)phenyl]ethyl]-5H,6H,7H-pyrrolo[3,4-d]pyrimidin-4-amine (1.0 g, 2.0 mmol), Cs₂CO₃ (976 mg, 3.0 mmol) and potassium vinyltrifluoroborate (348 mg, 2.60 mmol) in EtOH (40 mL). was added Pd(PPh₃)₄(231 mg, 0.2 mmol). The mixture was heated to 140° C. under microwave irradiation and stirred for 30 min, then filtered through a pad of Celite® and the filter cake washed with EtOAc. The filtrate was washed with H₂O and the aqueous layer was extracted with EtOAc (×3). The combined organic layers were dried over anhydrous Na₂SO₄, filtered, the solvent was concentrated under reduced pressure and the residue was purified by column chromatography to give 2-ethenyl-6-(morpholine-4-carbonyl)-N-[(1R)-1-[3-nitro-5-(trifluoromethyl)phenyl]ethyl]-5H,6H,7H-pyrrolo[3,4-d]pyrimidin-4-amine (682 mg, 69% yield). LCMS (ESI): m/z: [M+H] calculated for C₂₂H₂₃F₃N₆O₄: 492.17. found 593.1; ¹H NMR (300 MHz, DMSO-d₆) δ ppm 8.58 (s, 1H), 8.33 (d, J=1.8 Hz, 2H), 7.83 (d, J=7.2 Hz, 1H), 7.69-7.51 (m, 1H), 6.49 (dd, J=17.3, 10.3 Hz, 1H), 6.29 (dd, J=17.3, 2.5 Hz, 1H), 5.59-5.45 (m, 1H), 4.73-4.32 (m, 4H), 3.64 (t, J=4.6 Hz, 4H), 3.25 (t, J=4.7 Hz, 4H), 1.56 (d, J=7.0 Hz, 3H).

### Step 2.

To a mixture of 2-ethenyl-6-(morpholine-4-carbonyl)-N-[(1R)-1-[3-nitro-5-(trifluoromethyl)-phenyl]ethyl]-5H,6H,7H-pyrrolo[3,4-d]pyrimidin-4-amine (682 mg, 1.39 mmol) in THF (5.7 mL), acetone (5.7 mL) and H₂O (5.7 mL) was added 4-methylmorpholine N-oxide (649 mg, 5.54 mmol) and OsO₄, 2.5% wt in t-BuOH (0.59 mL, 0.058 mmol). The mixture was stirred at rt for 12 h, then a solution of sodium bisulfite was added and the mixture was extracted EtOAc (×2). The combined organic layers were dried over MgSO₄, filtered, the solvent was concentrated under reduced pressure and the residue was purified by column chromatography to give 1-[6-(morpholine-4-carbonyl)-4-{[(1R)-1-[3-nitro-5-(trifluoromethyl)phenyl]ethyl]amino}-5H,6H,7H-pyrrolo[3,4-d]pyrimidin-2-yl]ethane-1,2-diol (417 mg, 61% yield). LCMS (ESI): m/z: [M+H] calculated for C₂₂H₂₅F₃N₆O₆: 526.18. found 527.15; ¹H NMR (300 MHz, DMSO-d₆) δ ppm 8.64-8.57 (m, 1H), 8.34 (s, 1H), 8.32 (s, 1H), 7.86 (t, J=6.6 Hz, 1H), 5.60 (q, J=6.8 Hz, 1H), 4.71 (dd, J=28.0, 6.4 Hz, 1H), 4.64-4.55 (m, 2H), 4.52 (s, 2H), 4.46-4.37 (m, 1H), 4.35-4.19 (m, 1H), 3.63 (t, J=4.6 Hz, 4H), 3.25 (t, J=4.7 Hz, 4H), 1.55 (d, J=7.1 Hz, 3H).

### Step 3.

To a mixture of 1-[6-(morpholine-4-carbonyl)-4-{[(1R)-1-[3-nitro-5-(trifluoromethyl)phenyl]ethyl]-amino}-5H,6H,7H-pyrrolo[3,4-d]pyrimidin-2-yl]ethane-1,2-diol (417 mg, 0.79 mmol) in MeOH (6.5 mL) and H₂O (6.5 mL) was added NaIO₄ (508 mg, 2.38 mmol). The mixture was stirred at rt for 12 h, then H₂O was added and the mixture was extracted with EtOAc. The combined organic layers were dried over MgSO₄, filtered, the solvent was concentrated under reduced pressure and the residue was purified by column chromatography to give 6-(morpholine-4-carbonyl)-4-{[(1R)-1-[3-nitro-5-(trifluoromethyl)phenyl]ethyl]amino}-5H,6H,7H-pyrrolo[3,4-d]pyrimidine-2-carbaldehyde (287 mg, 73% yield). LCMS (ESI): m/z: [M+H] calculated for C₂₁H₂₁F₃N₆O₅: 494.15. found 495.00; ¹H NMR (300 MHz, DMSO-d₆) δ ppm 9.68 (s, 1H), 8.60 (s, 1H), 8.34 (s, 2H), 8.19 (d, J=7.5 Hz, 1H), 5.69-5.48 (m, 1H), 4.83-4.48 (m, 4H), 3.64 (t, J=4.6 Hz, 4H), 3.27 (t, J=4.6 Hz, 4H), 1.60 (d, J=7.1 Hz, 3H).

### Step 4.

To a mixture of 6-(morpholine-4-carbonyl)-4-{[(1R)-1-[3-nitro-5-(trifluoromethyl)phenyl]-ethyl]amino}-5H,6H,7H-pyrrolo[3,4-d]pyrimidine-2-carbaldehyde (90 mg, 0.18 mmol) in MeOH (0.6 mL) at 0° C. was added NaBH₄ (69 mg, 0.18 mmol). The mixture was stirred at 0° C. for 30 min, then ice-H₂O (10 mL) added and the solvent concentrated under reduced pressure. The residue was extracted with DCM, the combined organic layers were dried over MgSO₄, filtered and the solvent was concentrated under reduced pressure to give [6-(morpholine-4-carbonyl)-4-{[(1R)-1-[3-nitro-5-(trifluoromethyl)phenyl]ethyl]amino}-5H,6H,7H-pyrrolo[3,4-d]pyrimidin-2-yl]-methanol (82 mg, 91% yield). LCMS (ESI): m/z: [M+H] calculated for C₂₁H₂₃F₃N₆O₅: 496.17. found 497.6.

### Step 5.

(4-{[(1R)-1-[3-amino-5-(trifluoromethyl)-phenyl]ethyl]amino}-6-(morpholine-4-carbonyl)-5H,6H,7H-pyrrolo[3,4-d]pyrimidin-2-yl)methanol was synthesized in a manner similar to N-[(1R)-1-[3-amino-5-(trifluoromethyl)phenyl]ethyl]-2-chloro-7-(oxolan-3-ylmethyl)-5H,6H,7H,8H,9H-pyrimido[4,5-d]azepin-4-amine except 2-chloro-N-[(1R)-1-[3-nitro-5-(trifluoromethyl)phenyl]ethyl]-7-(oxolan-3-ylmethyl)-5H,6H,7H,8H,9H-pyrimido[4,5-d]azepin-4-amine was substituted with [6-(morpholine-4-carbonyl)-4-{[(1R)-1-[3-nitro-5-(trifluoromethyl)phenyl]ethyl]amino}-5H,6H,7H-pyrrolo[3,4-d]pyrimidin-2-yl]-methanol. LCMS (ESI): m/z: [M+H] calculated for C₂₁H₂₅F₃N₆O₃: 466.19. found 467.18; ¹H NMR (300 MHz, DMSO-d₆) δ ppm 7.64 (d, J=8.1 Hz, 1H), 6.84 (s, 1H), 6.79 (s, 1H), 6.68 (s, 1H), 5.57-5.49 (m, 2H), 5.46-5.30 (m, 1H), 4.70 (t, J=5.7 Hz, 1H), 4.61-4.47 (m, 4H), 4.30 (d, J=5.7 Hz, 2H), 3.70-3.57 (m, 4H), 3.29-3.19 (m, 4H), 1.44 (d, J=7.0 Hz, 3H).

### Example 348. (R)-(4-((1-(3-(difluoromethyl)-2-fluorophenyl)ethyl)amino)-2-(hydroxymethyl)-5,7-dihydro-6H-pyrrolo[3,4-d]pyrimidin-6-yl)(morpholino)methanone

(R)-(4-((1-(3-(difluoromethyl)-2-fluorophenyl)ethyl)amino)-2-(hydroxymethyl)-5,7-dihydro-6H-pyrrolo[3,4-d]pyrimidin-6-yl)(morpholino)methanone was synthesized in the manner similar to Example 129.

| | | Mass |
|---|---|---|
| Example # | Structure | found |
| Example 348. | 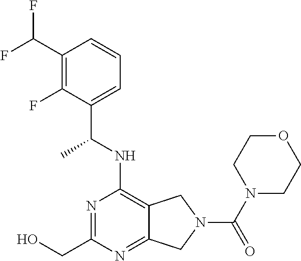 [SMILES: C[C@@H](Nc1nc(CO)nc2c1CN(C(=O)N1CCOCC1)C2)c1cccc(C(F)F)c1F] | 452.0 |

### Example 130. Synthesis of N-[(1R)-1-[3-amino-5-(trifluoromethyl)phenyl]ethyl]-2-(aminomethyl)-6-(morpholine-4-carbonyl)-5H,6H,7H-pyrrolo[3,4-d]pyrimidin-4-amine

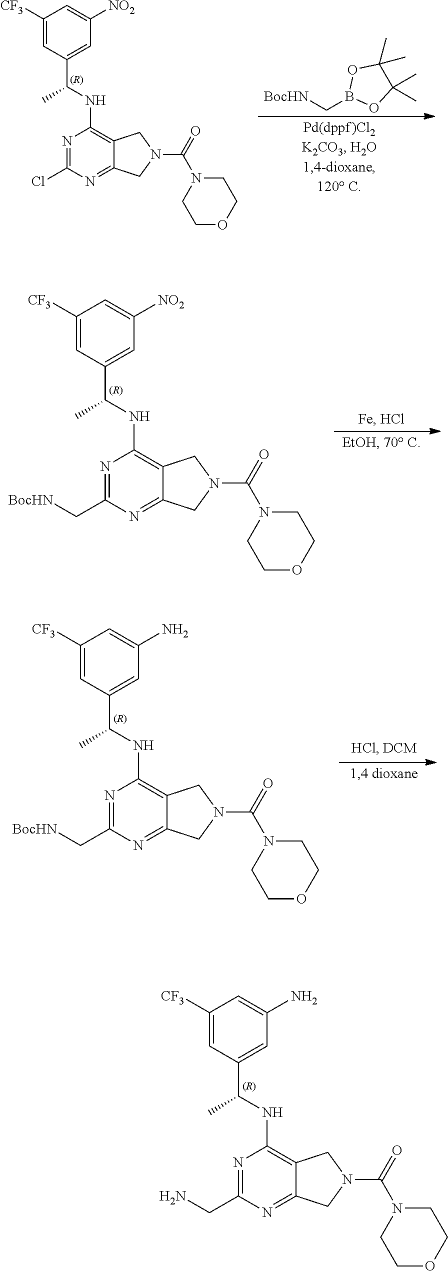

To an Ar-purged mixture of 2-chloro-6-(morpholine-4-carbonyl)-N-[(1R)-1-[3-nitro-5-(trifluoromethyl)phenyl]ethyl]-5H,6H,7H-pyrrolo[3,4-d]pyrimidin-4-amine (329 mg, 0.66 mmol) and N-Boc-aminomethylboronic acid pinacol ester (507 mg, 1.97 mmol) in 1,4-dioxane (6.6 mL) was added K₂CO₃ (182 mg, 1.31 mmol) in H₂O (3.9 mL). The mixture was purged with Ar for a further 15 min, then Pd(dppf)Cl₂ (48 mg, 66 μmol) was added. The mixture was heated to 120° C. under microwave irradiation and stirred for 2 h. Further N-Boc-aminomethylboronic acid pinacol ester (169 mg, 0.66 mmol) and Pd(dppf)Cl₂ (24 mg, 33 μmol) were added, the mixture was heated to 120° C. under microwave irradiation and stirred for 5 h. The mixture was filtered through a pad of Celite® and the filter cake was washed with MeOH. The solvent was concentrated under reduced pressure and the residue was purified by column chromatography to give tert-butyl N-{[6-(morpholine-4-carbonyl)-4-{[(1R)-1-[3-nitro-5-(trifluoromethyl)phenyl]ethyl]amino}-5H,6H,7H-pyrrolo[3,4-d]pyrimidin-2-yl]methyl}carbamate (440 mg). LCMS (ESI): m/z: [M+H] calculated for C₂₆H₃₂F₃N₇O₆: 595.24. found 596.05.

### Step 2.

Tert-butyl N-[(4-{[(1R)-1-[3-amino-5-(trifluoromethyl)phenyl]ethyl]amino}-6-(morpholine-4-carbonyl)-5H,6H,7H-pyrrolo[3,4-d]pyrimidin-2-yl)methyl]carbamate was synthesized in a manner similar to N-[(1R)-1-[3-amino-5-(trifluoromethyl)phenyl]ethyl]-2-chloro-7-(oxolan-3-ylmethyl)-5H,6H,7H,8H,9H-pyrimido[4,5-d]azepin-4-amine except 2-chloro-N-[(1R)-1-[3-nitro-5-(trifluoromethyl)phenyl]ethyl]-7-(oxolan-3-ylmethyl)-5H,6H,7H,8H,9H-pyrimido[4,5-d]azepin-4-amine was substituted with tert-butyl N-{[6-(morpholine-4-carbonyl)-4-{[(1R)-1-[3-nitro-5-(trifluoromethyl)phenyl]ethyl]amino}-5H,6H,7H-pyrrolo[3,4-d]pyrimidin-2-yl]methyl}carbamate. LCMS (ESI): m/z: [M+H] calculated for C₂₆H₃₄F₃N₇O₄: 565.60. found 566.20.

### Step 3.

To a mixture of tert-butyl N-[(4-{[(1R)-1-[3-amino-5-(trifluoromethyl)phenyl]ethyl]amino}-6-(morpholine-4-carbonyl)-5H,6H,7H-pyrrolo[3,4-d]pyrimidin-2-yl)methyl]carbamate (325 mg, 0.57 mmol) in DCM (4.9 mL) at 0° C. was added 4M HCl in 1,4-dioxane (1.9 mL, 7.5 mmol). The mixture was allowed to warm to rt and stirred overnight, then partitioned between DCM and saturated NaHCO₃. The aqueous phase was extracted with DCM (×2), the combined organic layers were dried over anhydrous Na₂SO₄ and filtered. The solvent was concentrated under reduced pressure and the residue was purified by prep-HPLC to give N-[(1R)-1-[3-amino-5-(trifluoromethyl)phenyl]ethyl]-2-(aminomethyl)-6-(morpholine-4-carbonyl)-5H,6H,7H-pyrrolo[3,4-d]pyrimidin-4-amine (50 mg, 19% yield). LCMS (ESI): m/z: [M+H] calculated for C₂₁H₂₆F₃N₇O₂: 465.21. found 465.93; H NMR (300 MHz, DMSO-d₆) δ ppm 7.59 (d, J=7.7 Hz, 1H), 6.84 (s, 1H), 6.79 (s, 1H), 6.67 (s, 1H), 5.52 (s, 2H), 5.37-5.26 (m, 1H), 4.59-4.45 (m, 4H), 3.63 (t, J=4.6 Hz, 4H), 3.55 (s, 2H), 3.24 (t, J=4.7 Hz, 4H), 2.02 (br s, 2H), 1.44 (d, J=7.0 Hz, 3H).

### Example 349. (R)-(2-(aminomethyl)-4-((1-(3-(difluoromethyl)phenyl)ethyl)amino)-5,7-dihydro-6H-pyrrolo[3,4-d]pyrimidin-6-yl)(morpholino)methanone

(R)-(2-(aminomethyl)-4-((1-(3-(difluoromethyl)phenyl)ethyl)amino)-5,7-dihydro-6H-pyrrolo[3,4-d]pyrimidin-6-yl)(morpholino)methanone was synthesized in the manner similar to Example 130.

| | | Mass |
|---|---|---|
| Example # | Structure | Found |
| Example 349. | 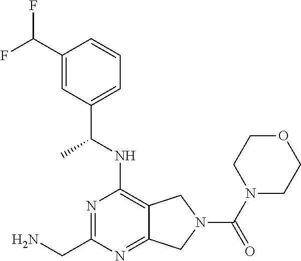 [SMILES: C[C@@H](Nc1nc(CN)nc2c1CN(C(=O)N1CCOCC1)C2)c1cccc(C(F)F)c1] | 451.0 |

### Example 350. (R)-(2-(aminomethyl)-4-((1-(3-(trifluoromethyl)phenyl)ethyl)amino)-5,7-dihydro-6H-pyrrolo[3,4-d]pyrimidin-6-yl)(4-methoxytetrahydro-2H-pyran-4-yl)methanone

(R)-(2-(aminomethyl)-4-((1-(3-(trifluoromethyl)phenyl)ethyl)amino)-5,7-dihydro-6H-pyrrolo[3,4-d]pyrimidin-6-yl)(4-methoxytetrahydro-2H-pyran-4-yl)methanone was synthesized in the manner similar to Example 130.

| | | Mass |
|---|---|---|
| Example # | Structure | Found |
| Example 350. | 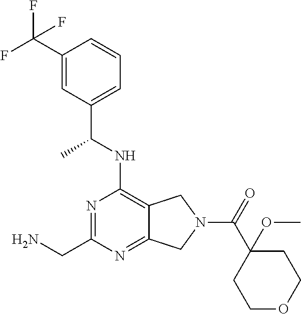 [SMILES: COC1(C(=O)N2Cc3nc(CN)nc(N[C@H](C)c4cccc(C(F)(F)F)c4)c3C2)CCOCC1] | 480.1 |

### Example 131. Synthesis of N-[(1R)-1-[3-amino-5-(trifluoro-methyl)phenyl]ethyl]-2-bromo-6-(morpholine-4-carbonyl)-5H,6H,7H-pyrrolo[3,4-d]pyrimidin-4-amine

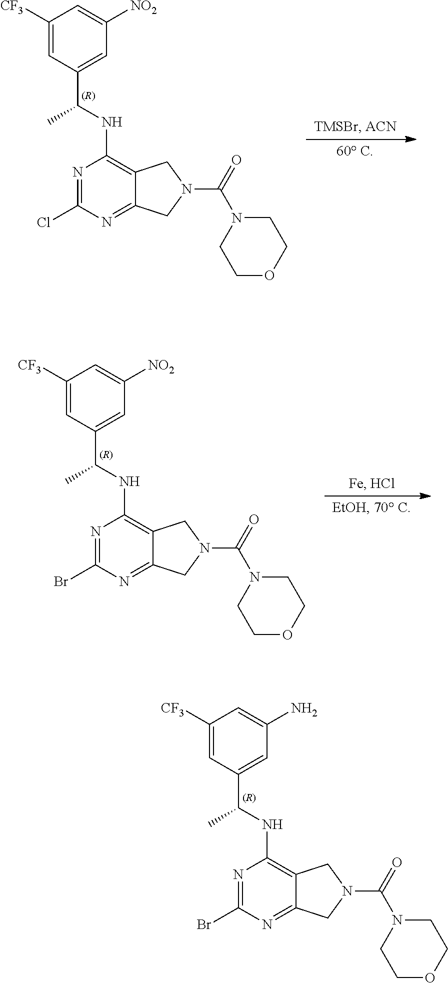

### Step 1.

To a mixture of 2-chloro-6-(morpholine-4-carbonyl)-N-[(1R)-1-[3-nitro-5-(trifluoromethyl)phenyl]-ethyl]-5H,6H,7H-pyrrolo[3,4-d]pyrimidin-4-amine (200 mg, 0.4 mmol) in MeCN (5 mL) was added TMSBr (122 mg, 0.8 mmol). The mixture was heated to 60° C. and stirred overnight. The solvent was concentrated under reduced pressure, the residue was diluted with aqueous NaHCO₃ and extracted with EtOAc (×3). The combined organic layers were dried over anhydrous Na₂SO₄, filtered and the solvent was concentrated under reduced pressure to give 2-bromo-6-(morpholine-4-carbonyl)-N-[(1R)-1-[3-nitro-5-(trifluoromethyl)phenyl]ethyl]-5H,6H,7H-pyrrolo[3,4-d]pyrimidin-4-amine (248 mg). LCMS (ESI): m/z: [M+H] calculated for C₂₀H₂₀BrF₃N₆O₄: 544.07. found 546.80; ¹H NMR (300 MHz, DMSO-d₆) δ ppm 8.55 (t, J=1.9 Hz, 1H), 8.37 (d, J=2.0 Hz, 1H), 8.27 (s, 2H), 5.53-5.34 (m, 1H), 4.52 (q, J=14.5, 13.3 Hz, 4H), 3.62 (t, J=4.6 Hz, 4H), 3.24 (t, J=4.6 Hz, 4H), 1.55 (d, J=6.8 Hz, 3H).

N-[(1R)-1-[3-amino-5-(trifluoro-methyl)phenyl]ethyl]-2-bromo-6-(morpholine-4-carbonyl)-5H,6H,7H-pyrrolo[3,4-d]pyrimidin-4-amine was synthesized in a manner similar to N-[(1R)-1-[3-amino-5-(trifluoromethyl)phenyl]ethyl]-2-chloro-7-(oxolan-3-ylmethyl)-5H,6H,7H,8H,9H-pyrimido[4,5-d]azepin-4-amine except 2-chloro-N-[(1R)-1-[3-nitro-5-(trifluoromethyl)phenyl]ethyl]-7-(oxolan-3-ylmethyl)-5H,6H,7H,8H,9H-pyrimido[4,5-d]azepin-4-amine was substituted with 2-bromo-6-(morpholine-4-carbonyl)-N-[(1R)-1-[3-nitro-5-(trifluoromethyl)phenyl]ethyl]-5H,6H,7H-pyrrolo[3,4-d]pyrimidin-4-amine. LCMS (ESI): m/z: [M+H] calculated for C₂₀H₂₂BrF₃N₆O₂: 514.09. found 515.04; H NMR (300 MHz, DMSO-d₆) δ ppm 8.09 (d, J=7.6 Hz, 1H), 6.81 (s, 1H), 6.76 (s, 1H), 6.71 (d, J=1.9 Hz, 1H), 5.57 (s, 2H), 5.18 (t, J=7.1 Hz, 1H), 4.64-4.38 (m, 4H), 3.62 (t, J=4.6 Hz, 4H), 3.23 (t, J=4.7 Hz, 4H), 1.44 (d, J=7.0 Hz, 3H).

### Example 132. Synthesis of N-[(1R)-1-[3-amino-5-(trifluoromethyl)phenyl]ethyl]-2-(difluoromethyl)-6-(morpholine-4-carbonyl)-5H,6H,7H-pyrrolo[3,4-d]pyrimidin-4-amine

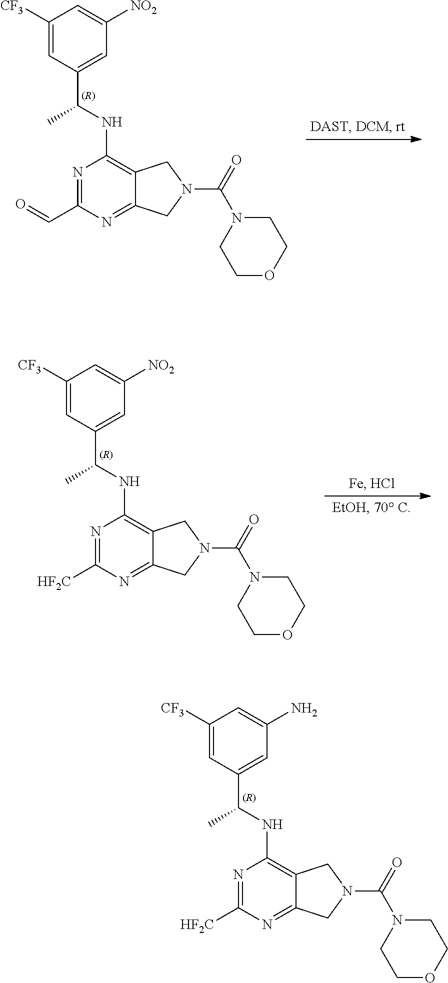

### Step 1.

To a mixture of 6-(morpholine-4-carbonyl)-4-{[(1R)-1-[3-nitro-5-(trifluoromethyl)phenyl]ethyl]amino}-5H,6H,7H-pyrrolo[3,4-d]pyrimidine-2-carbaldehyde (257 mg, 0.52 mmol) in DCM (5.1 mL) was added DAST (42 mg, 0.26 mmol). The mixture was stirred at rt overnight, then saturated NaHCO₃ was added and the mixture extracted with DCM. The combined organic layers were dried over anhydrous Na₂SO₄, filtered and the solvent concentrated under reduced pressure to give 2-(difluoromethyl)-6-(morpholine-4-carbonyl)-N-[(1R)-1-[3-nitro-5-(trifluoromethyl)phenyl]ethyl]-5H,6H,7H-pyrrolo[3,4-d]pyrimidin-4-amine (219 mg, 82% yield). LCMS (ESI): m/z: [M+H] calculated for C₂₁H₂₁F₅N₆O₄: 516.15. found 517.0.

### Step 2.

N-[(1R)-1-[3-amino-5-(trifluoromethyl)phenyl]ethyl]-2-(difluoromethyl)-6-(morpholine-4-carbonyl)-5H,6H,7H-pyrrolo[3,4-d]pyrimidin-4-amine was synthesized in a manner similar to N-[(1R)-1-[3-amino-5-(trifluoromethyl)phenyl]ethyl]-2-chloro-7-(oxolan-3-ylmethyl)-5H,6H,7H,8H,9H-pyrimido[4,5-d]azepin-4-amine except 2-chloro-N-[(1R)-1-[3-nitro-5-(trifluoromethyl)phenyl]ethyl]-7-(oxolan-3-ylmethyl)-5H,6H,7H,8H,9H-pyrimido[4,5-d]azepin-4-amine was substituted with 2-(difluoromethyl)-6-(morpholine-4-carbonyl)-N-[(1R)-1-[3-nitro-5-(trifluoromethyl)phenyl]ethyl]-5H,6H,7H-pyrrolo[3,4-d]pyrimidin-4-amine. LCMS (ESI): m/z: [M+H] calculated for C₂₁H₂₃F₅N₆O₂: 486.18. found 487.19; ¹H NMR (400 MHz, DMSO-d₆) δ ppm 8.00 (d, J=8.0 Hz, 1H), 6.85 (s, 1H), 6.80 (s, 1H), 6.70 (s, 1H), 6.59 (t, J=54.6 Hz, 1H), 5.55 (s, 2H), 5.38-5.25 (m, 1H), 4.65-4.53 (m, 4H), 3.64 (t, J=4.6 Hz, 4H), 3.26 (t, J=4.6 Hz, 4H), 1.48 (d, J=7.0 Hz, 3H); ¹⁹F NMR (376 MHz, DMSO-d₆) δ ppm −61.3, −118.2 (dd, J=54.6, 6.1 Hz).

### Example 133. Synthesis of N-[(1R)-1-[3-amino-5-(trifluoro-methyl)phenyl]ethyl]-6-(morpholine-4-carbonyl)-2-(oxetan-3-yl-oxy)-5H,6H,7H-pyrrolo[3,4-d]pyrimidin-4-amine

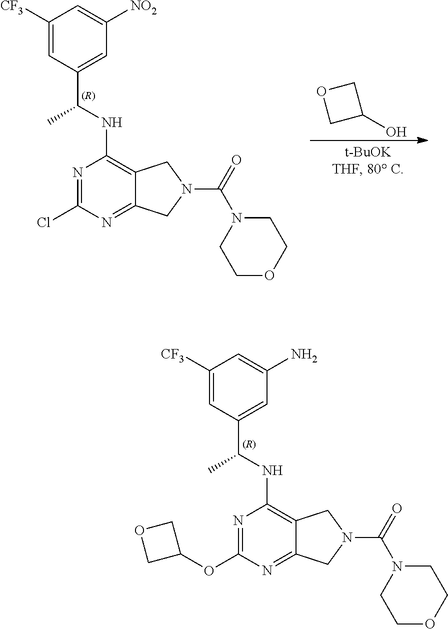

### Step 1.

To a mixture of 3-oxetanol (222 mg, 2.99 mmol) in THF (8.6 mL) was added potassium tert-butoxide (363 mg, 2.99 mmol). The mixture was heated to 50° C. and stirred for 15 min, then cooled to rt and (R)-(4-((1-(3-amino-5-(trifluoromethyl)phenyl)ethyl)amino)-2-chloro-5,7-dihydro-6H-pyrrolo[3,4-d]pyrimidin-6-yl)(morpholino)methanone (300 mg, 0.6 mmol) in THF (5 mL). The mixture was heated to 80° C. and stirred overnight, then H₂O was added and the mixture was extracted with EtOAc. The combined organic layers were dried over anhydrous Na₂SO₄, filtered, and the solvent was removed under reduced pressure. The crude product was triturated with i-PrOH, filtered, the solvent was concentrated under reduced pressure and the residue was purified by prep-HPLC to give N-[(1R)-1-[3-amino-5-(trifluoro-methyl)phenyl]ethyl]-6-(morpholine-4-carbonyl)-2-(oxetan-3-yl-oxy)-5H,6H,7H-pyrrolo[3,4-d]pyrimidin-4-amine (11.7 mg, 4% yield). LCMS (ESI): m/z: [M+H] calculated for C₂₃H₂₇F₃N₆O₄: 508.20. found 509.22; ¹H NMR (300 MHz, DMSO-d₆) δ ppm 7.77 (d, J=7.5 Hz, 1H), 6.80 (s, 1H), 6.74 (s, 1H), 6.68 (s, 1H), 5.56 (s, 2H), 5.28 (t, J=5.6 Hz, 1H), 5.15-5.04 (m, 1H), 4.81 (t, J=6.9 Hz, 1H), 4.60 (t, J=7.0 Hz, 1H), 4.55-4.45 (m, 3H), 4.43 (s, 2H), 4.34-4.23 (m, 1H), 3.62 (t, J=4.7 Hz, 4H), 3.23 (t, J=4.7 Hz, 4H), 1.42 (d, J=7.0 Hz, 3H).

### Example 134. Synthesis of N-[(1R)-1-[3-amino-5-(trifluoromethyl)phenyl]ethyl]-2-iodo-6-(morpholine-4-carbonyl)-5H,6H,7H-pyrrolo[3,4-d]pyrimidin-4-amine

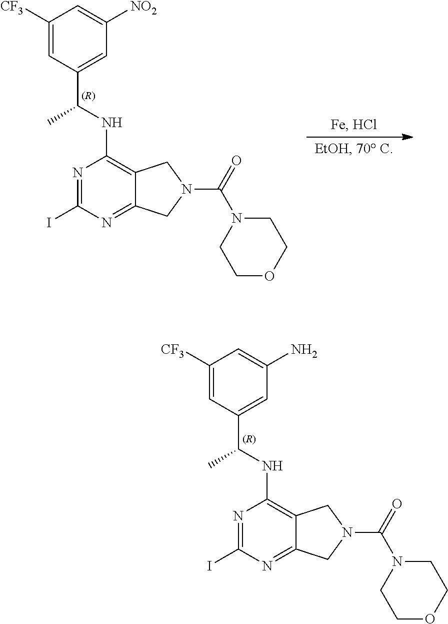

### Step 1.

N-[(1R)-1-[3-amino-5-(trifluoromethyl)phenyl]ethyl]-2-iodo-6-(morpholine-4-carbonyl)-5H,6H,7H-pyrrolo[3,4-d]pyrimidin-4-amine was synthesized in a manner similar to N-[(1R)-1-[3-amino-5-(trifluoromethyl)phenyl]ethyl]-2-chloro-7-(oxolan-3-ylmethyl)-5H,6H,7H,8H,9H-pyrimido[4,5-d]azepin-4-amine except 2-chloro-N-[(1R)-1-[3-nitro-5-(trifluoromethyl)phenyl]ethyl]-7-(oxolan-3-ylmethyl)-5H,6H,7H,8H,9H-pyrimido[4,5-d]azepin-4-amine was substituted with a 1:1 mixture of 2-iodo-6-(morpholine-4-carbonyl)-N-[(1R)-1-[3-nitro-5-(trifluoromethyl)phenyl]ethyl]-5H,6H,7H-pyrrolo[3,4-d]pyrimidin-4-amine and 2-chloro-6-(morpholine-4-carbonyl)-N-[(1R)-1-[3-nitro-5-(trifluoromethyl)phenyl]ethyl]-5H,6H,7H-pyrrolo[3,4-d]pyrimidin-4-amine. LCMS (ESI): m/z: [M+H] calculated for C₂₁H₂₂F₆N₆O₂: 562.33. found 563.11; ¹H NMR (400 MHz, DMSO-d₆) δ ppm 7.98 (d, J=7.8 Hz, 1H), 6.82 (s, 1H), 6.75 (s, 1H), 6.71 (s, 1H), 5.57 (s, 2H), 5.16 (t, J=7.4 Hz, 1H), 4.49 (d, J=15.4 Hz, 4H), 3.61 (t, J=4.6 Hz, 4H), 3.23 (t, J=4.8 Hz, 4H), 1.44 (d, J=7.0 Hz, 3H).

### Example 135. Synthesis of N-{[3-amino-5-(trifluoromethyl)phenyl]-methyl}-2-chloro-6-(morpholine-4-carbonyl)-5H,6H,7H-pyrrolo-[3,4-d]pyrimidin-4-amine

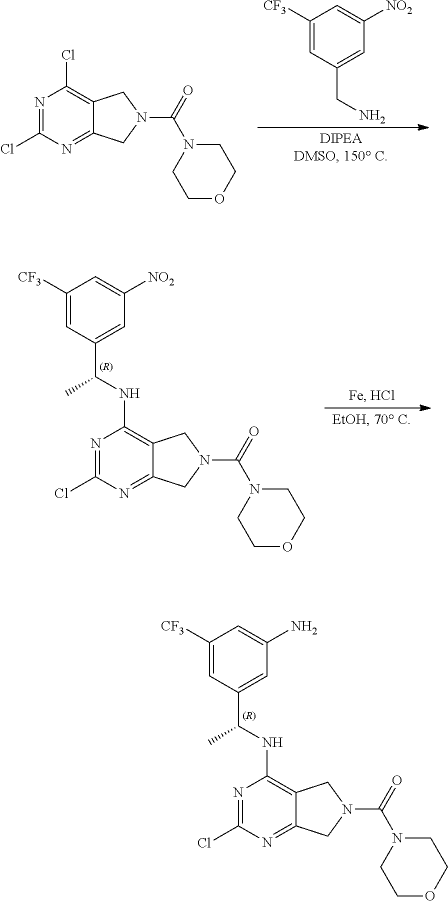

### Step 1.

A mixture of 4-{2,4-dichloro-5H,6H,7H-pyrrolo[3,4-d]pyrimidine-6-carbonyl}morpholine (250 mg, 0.82 mmol) and [3-nitro-5-(trifluoromethyl)phenyl]methanamine HCl salt (222 mg, 0.87 mmol) in anhydrous DMSO (7.5 mL) was purged with Ar. DIPEA (575 μL, 3.3 mmol) was added and the mixture was heated to 150° C. under microwave irradiation for 1 h. H₂O and Et₂O were added and the aqueous layer extracted with Et₂O (×3). The combined organic layers were washed with H₂O, dried over anhydrous Na₂SO₄, filtered and the solvent was concentrated under reduced pressure to give 2-chloro-6-(morpholine-4-carbonyl)-N-{[3-nitro-5-(trifluoromethyl)-phenyl]methyl}-5H,6H,7H-pyrrolo[3,4-d]pyrimidin-4-amine (200 mg, 50% yield). LCMS (ESI): m/z: [M+H] calculated for C₁₉H₁₈ClF₃N₆O₄: 486.10. found 487.10.

### Step 2.

Fe powder (126 mg, 2.26 mmol) and 1M HCl (1.64 mL, 1.64 mmol) were added to a mixture of 2-chloro-6-(morpholine-4-carbonyl)-N-{[3-nitro-5-(trifluoromethyl)-phenyl]methyl}-5H,6H,7H-pyrrolo[3,4-d]pyrimidin-4-amine (200 mg, 0.41 mmol) in EtOH (4.1 mL). The mixture was heated to 70° C. and stirred overnight. The mixture was filtered through Celite®, the filter cake was washed with MeOH and the solvent was concentrated under reduced pressure. The residue was dissolved in DCM, washed with NaHCO₃ and the aqueous layer extracted with DCM (×3). The combined organic layers were dried over anhydrous Na₂SO₄, the solvent was concentrated under reduced pressure and the residue was purified by prep-HPLC to give N-{[3-amino-5-(trifluoromethyl)phenyl]methyl}-2-chloro-6-(morpholine-4-carbonyl)-5H,6H,7H-pyrrolo[3,4-d]pyrimidin-4-amine (32 mg, 17% yield). LCMS (ESI): m/z: [M+H] calculated for C₁₉H₂₀ClF₃N₆O₂: 456.13. found 457.14; ¹H NMR (300 MHz, DMSO-d₆) δ ppm 8.31 (t, J=5.7 Hz, 1H), 6.81-6.41 (m, 3H), 5.60 (s, 2H), 4.59-4.43 (m, 6H), 3.61 (t, J=4.6 Hz, 4H), 3.23 (t, J=4.2 Hz, 4H).

### Example 136. Synthesis of N-[(1R)-1-[3-amino-5-(trifluoromethyl)phenyl]ethyl]-2-methyl-6-(morpholine-4-carbonyl)-5H,6H,-7H,8H,9H-pyrimido[5,4-c]azepin-4-amine

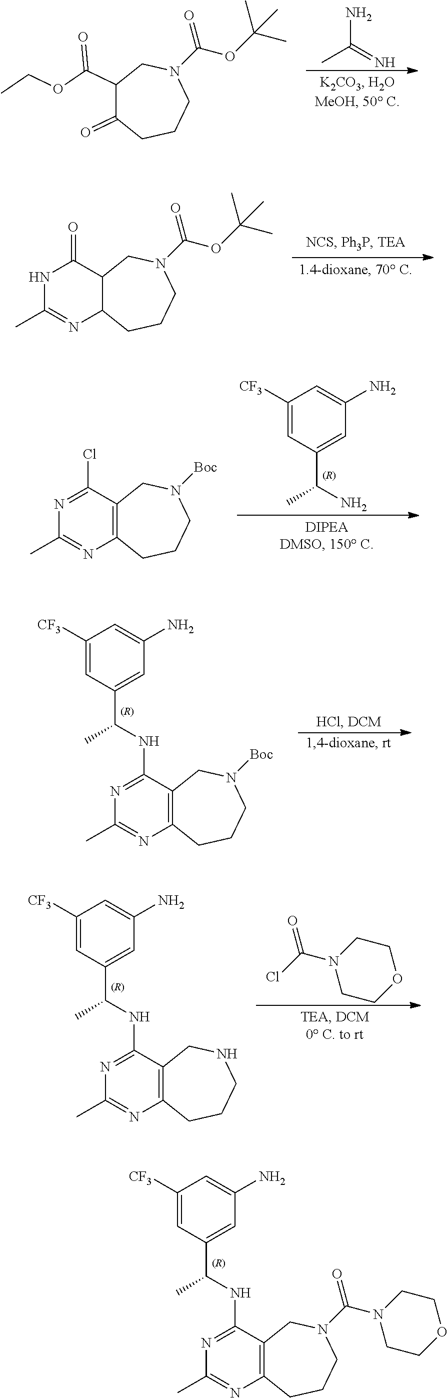

### Step 1.

A mixture of 1-tert-butyl 3-ethyl 4-oxoazepane-1,3-dicarboxylate (3.0 g, 10.5 mmol), acetamidine HCl salt (1.19 g, 12.61 mmol) and K₂CO₃ (2.18 g, 15.77 mmol) in MeOH (42 mL) and H₂O (12 mL) under an atmosphere of Ar was heated to 50° C. and stirred overnight. The mixture was acidified with 1M HCl and extracted with DCM (9×100 mL). The combined organic layers were washed with brine, dried over anhydrous Na₂SO₄ and filtered. The solvent was concentrated under reduced pressure and the residue was purified by column chromatography to give tert-butyl 2-methyl-4-oxo-3H,4H,5H,6H,7H,8H,9H-pyrimido[5,4-c]azepine-6-carboxylate (1.89 g, 64% yield). LCMS (ESI): m/z: [M+H] calculated for C₁₄H₂₁N₃O₃: 279.16. found 280.15; ¹H NMR (300 MHz, CDCl₃) δ ppm 13.09 (s, 1H), 4.54-4.38 (m, 2H), 3.69-3.62 (m, 2H), 2.96-2.85 (m, 2H), 2.47 (s, 3H), 1.99-1.88 (m, 2H), 1.43 (s, 9H).

### Step 2.

A mixture of Ph₃P (1.83 g, 6.98 mmol) and N-chlorosuccinimide (0.93 g, 6.98 mmol) in 1,4-dioxane (39 mL) was stirred under an atmosphere of Ar at rt for 30 min. Tert-butyl 2-methyl-4-oxo-3H,4H,5H,6H,7H,8H,9H-pyrimido[5,4-c]azepine-6-carboxylate (1.3 g, 4.65 mmol) was added and the mixture was heated to 70° C. and stirred for 4 h. Et₃N (130 μL) was added and the solvent was concentrated under reduced pressure. The residue was partitioned between H₂O and DCM and the aqueous layer was extracted with DCM (3×40 mL). The combined organic layers were dried over anhydrous Na₂SO₄, filtered, the solvent was concentrated under reduced pressure and the residue was purified by column chromatography to give tert-butyl 4-chloro-2-methyl-5H,6H,7H,8H,9H-pyrimido[5,4-c]azepine-6-carboxylate (1.14 g, 82% yield). LCMS (ESI): m/z: [M+H] calculated for C₁₄H₂₀ClN₃O₂: 297.12. found 298.05; ¹H NMR (300 MHz, DMSO-d₆) δ ppm 4.70-4.59 (m, 2H), 3.62 (t, J=5.6 Hz, 2H), 3.11-2.98 (m, 2H), 2.53 (s, 3H), 1.83-1.72 (m, 2H), 1.32 (s, 9H).

### Step 3.

A mixture of tert-butyl 4-chloro-2-methyl-5H,6H,7H,8H,9H-pyrimido[5,4-c]azepine-6-carboxylate (850 mg, 2.86 mmol) and 3-(1-aminoethyl)-5-(trifluoromethyl)aniline HCl salt (723 mg, 3.0 mmol) were dissolved in DMSO (17 mL). The mixture was purged with Ar and DIPEA (2 mL, 11.4 mmol) was added in one portion. The mixture was heated to 150° C. under microwave irradiation and stirred for 5h, then diluted with Et₂O and H₂O and the aqueous layer extracted with Et₂O (16×50 mL). The combined organic layers were dried over anhydrous Na₂SO₄, filtered, the solvent was concentrated under reduced pressure and the residue was purified by column chromatography to give tert-butyl 4-{[(1R)-1-[3-amino-5-(trifluoromethyl)phenyl]ethyl]amino}-2-methyl-5H,6H,7H,8H,9H-pyrimido[5,4-c]azepine-6-carboxylate (140 mg, 11% yield). LCMS (ESI): m/z: [M+H] calculated for C₂₃H₃₀F₃N₅O₂: 465.23. found 466.4.

### Step 4.

To a mixture of tert-butyl 4-{[(1R)-1-[3-amino-5-(trifluoromethyl)phenyl]ethyl]amino}-2-methyl-5H,6H,7H,8H,9H-pyrimido[5,4-c]azepine-6-carboxylate (130 mg, 0.28 mmol) in DCM (2.3 mL) under an atmosphere of Ar was added 4M HCl in 1,4-dioxane (0.9 mL, 3.63 mmol). The mixture was stirred at rt overnight and the solvent was concentrated under reduced pressure to give N-[(1R)-1-[3-amino-5-(trifluoromethyl)phenyl]ethyl]-2-methyl-5H,6H,7H,8H,9H-pyrimido[5,4-c]azepin-4-amine HCl salt (86 mg, 84% yield). LCMS (ESI): m/z: [M+H] calculated for C₁₈H₂₂F₃N₅: 365.18. found 366.25; ¹H NMR (300 MHz, DMSO-d₆) δ ppm 9.50 (m, 1H), 7.13 (s, 1H), 7.08 (s, 1H), 6.94 (d, J=10.9 Hz, 1H), 6.89 (s, 1H), 5.52-5.44 (m, 1H), 4.53-4.48 (m, 2H), 3.40-3.33 (m, 2H), 3.19-3.14 (m, 2H), 1.98-1.91 (m, 2H), 1.57 (d, J=7.0 Hz, 3H), 1.35 (s, 3H).

### Step 5.

To a mixture of N-[(1R)-1-[3-amino-5-(trifluoromethyl)phenyl]ethyl]-2-methyl-5H,6H,7H,8H,9H-pyrimido[5,4-c]azepin-4-amine HCl salt (0.12 g, 0.33 mmol) in DCM (3.6 mL) and Et₃N (0.23 mL, 1.64 mmol) at 0° C. under an atmosphere of Ar was added a solution of morpholine-4-carbonyl chloride (52 mg, 0.34 mmol) in DCM (0.2 mL). The mixture was warmed to rt and stirred overnight, then the solvent was concentrated under reduced pressure and the residue was purified by prep-HPLC to give N-[(1R)-1-[3-amino-5-(trifluoromethyl)phenyl]ethyl]-2-methyl-6-(morpholine-4-carbonyl)-5H,6H,-7H,8H,9H-pyrimido[5,4-c]azepin-4-amine (27 mg, 17% yield). LCMS (ESI): m/z: [M+H] calculated for C₂₃H₂₉F₃N₆O₂: 478.23. found 479.2; ¹H NMR (300 MHz, DMSO-d₆) δ ppm 7.05 (d, J=7.9 Hz, 1H), 6.92 (s, 1H), 6.85 (s, 1H), 6.67 (s, 1H), 5.46 (s, 2H), 5.28 (t, J=7.2 Hz, 1H), 4.22 (s, 2H), 3.57-3.46 (m, 6H), 3.04-2.97 (m, 5H), 2.77 (d, J=8.7 Hz, 3H), 2.22 (s, 3H), 1.83-1.71 (m, 2H), 1.44 (d, J=7.0 Hz, 3H).

### Example 137. Synthesis of 2-chloro-6-(morpholine-4-carbonyl)-N-[(1R)-1-[3-(trifluoromethyl)phenylethyl-5H,6H,7H-pyrrolo[3,4-d]pyrimidin-4-amine

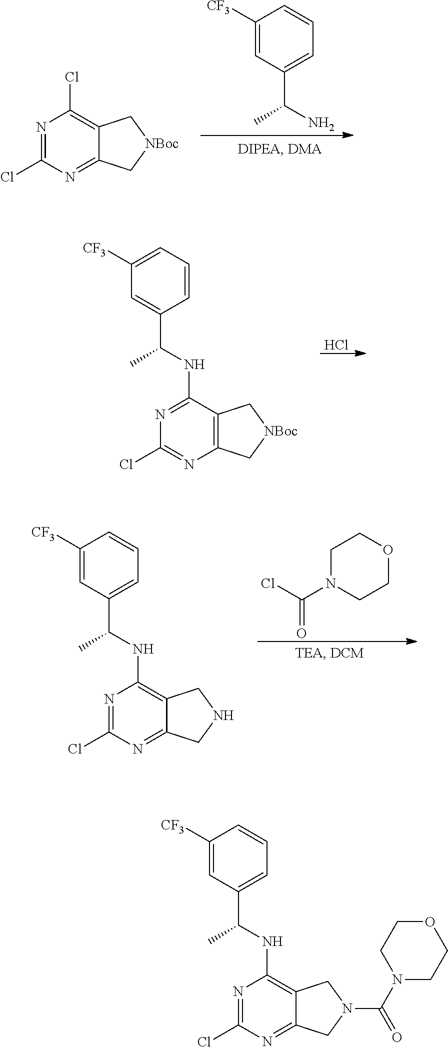

### Step 1.

To a mixture of (1R)-1-[3-(trifluoromethyl)phenyl]ethan-1-amine HCl salt (388 mg, 1.71 mmol) and tert-butyl 2,4-dichloro-5H,6H,7H-pyrrolo[3,4-d]pyrimidine-6-carboxylate (500 mg, 1.72 mmol) in DMA (1.72 mL) was added DIPEA (598 μL, 3.44 mmol). The mixture was stirred at rt for 1 h, then diluted with H₂O and extracted with EtOAc. The combined organic layers were washed with brine, dried over MgSO₄, filtered and the solvent was removed under reduced pressure to give tert-butyl 2-chloro-4-{[(1R)-1-[3-(trifluoromethyl)phenyl]ethyl]amino}-5H,6H,7H-pyrrolo[3,4-d]pyrimidine-6-carboxylate (673 mg), which was used in the next step without further purification. LCMS (ESI): m/z: [M+H] calculated for C₂₀H₂₃ClF₃N₄O₂: 443.2. found 443.4.

### Step 2.

Tert-butyl 2-chloro-4-{[(1R)-1-[3-(trifluoromethyl)phenyl]ethyl]amino}-5H,6H,7H-pyrrolo[3,4-d]pyrimidine-6-carboxylate (673 mg, 1.51 mmol) was dissolved in 4M HCl in 1,4-dioxane/MeOH (3 mL). The mixture was heated to 40° C. and stirred for 1 h, then the solvent was removed under reduced pressure and the crude residue was purified by column chromatography to give 2-chloro-N-[(1R)-1-[3-(trifluoromethyl)phenyl]ethyl]-5H,6H,7H-pyrrolo[3,4-d]pyrimidin-4-amine (181 mg, 35% yield). LCMS (ESI): m/z: [M+H] calculated for C₁₅H₁₅ClF₃N₄: 343.1. found 343.2.

### Step 3.

To a mixture of 2-chloro-N-[(1R)-1-[3-(trifluoromethyl)phenyl]ethyl]-5H,6H,7H-pyrrolo[3,4-d]pyrimidin-4-amine (90 mg, 0.26 mmol) in DCM (0.7 mL) at 0° C. was added a solution of morpholine-4-carbonyl chloride (1 mL; 0.79 mmol) and Et₃N (180 L, 1.30 mmol). The mixture was allowed to warm to rt and stirred for 1 h, then the solvent was removed under reduced pressure and the crude residue was purified by prep-HPLC to give 2-chloro-6-(morpholine-4-carbonyl)-N-[(1R)-1-[3-(trifluoromethyl)phenyl]ethyl]-5H,6H,7H-pyrrolo[3,4-d]pyrimidin-4-amine (29 mg, 24% o yield). LCMS (ESI): m/z: [M+H] calculated for C₂₀H₂₂ClF₃N₅O₂: 456.1. found 456.5; ¹H NMR (500 MHz, METHANOL-dL₄) δ ppm 7.71 (d, J=2.1 Hz, 1H), 7.68-7.64 (m, 1H), 7.56-7.49 (m, 2H), 5.43 (q, J=7.1 Hz, 1H), 4.62 (d, J=2.5 Hz, 2H), 4.56 (dt, J=5.0, 2.2 Hz, 2H), 3.74-3.69 (m, 4H), 3.37-3.33 (m, 4H), 1.59 (d, J=7.0 Hz, 3H).

The examples in the following Table 5 were synthesized in the manner similar to Example 137.

TABLE 5
Examples 138-164, 351-430, 490-499, and 537

| Example # | Structure | Mass found |
|---|---|---|
| Example 138. | 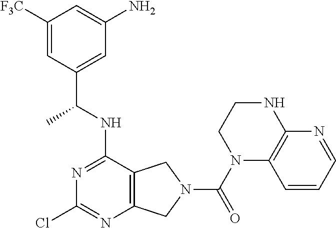 [SMILES: C[C@@H](Nc1nc(Cl)nc2c1CN(C(=O)N1CCNc3ncccc31)C2)c1cc(N)cc(C(F)(F)F)c1] | 519.5 |
| Example 139. | 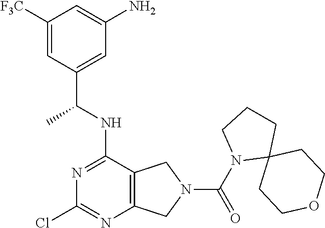 [SMILES: C[C@@H](Nc1nc(Cl)nc2c1CN(C(=O)N1CCCC13CCOCC3)C2)c1cc(N)cc(C(F)(F)F)c1] | 525.5 |
| Example 140. | 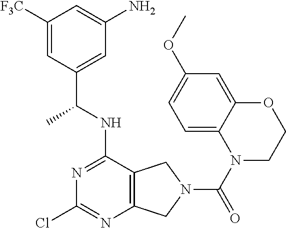 [SMILES: COc1ccc2c(c1)OCCN2C(=O)N1Cc2nc(Cl)nc(N[C@H](C)c3cc(N)cc(C(F)(F)F)c3)c2C1] | 549.6 |
| Example 141. | 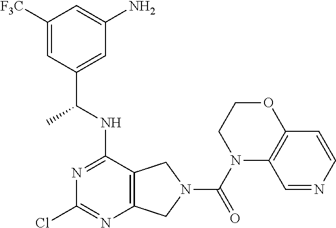 [SMILES: C[C@@H](Nc1nc(Cl)nc2c1CN(C(=O)N1CCOc3ccncc31)C2)c1cc(N)cc(C(F)(F)F)c1] | 520.5 |
| Example 142. | 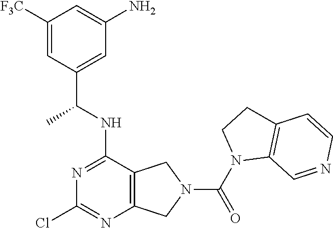 [SMILES: C[C@@H](Nc1nc(Cl)nc2c1CN(C(=O)N1CCc3ccncc31)C2)c1cc(N)cc(C(F)(F)F)c1] | 504.5 |
| Example 143. | 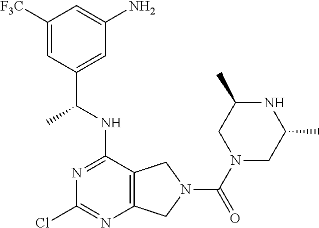 [SMILES: C[C@@H]1CN(C(=O)N2Cc3nc(Cl)nc(N[C@H](C)c4cc(N)cc(C(F)(F)F)c4)c3C2)C[C@@H](C)N1] | 498.6 |
| Example 144. | 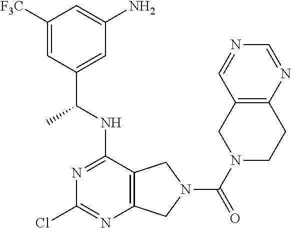 [SMILES: C[C@@H](Nc1nc(Cl)nc2c1CN(C(=O)N1CCc3ncncc3C1)C2)c1cc(N)cc(C(F)(F)F)c1] | 519.28 |
| Example 145. | 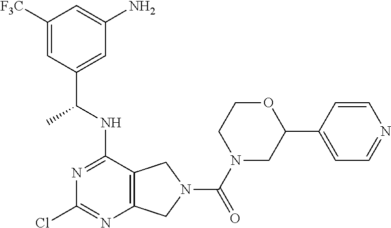 [SMILES: C[C@@H](Nc1nc(Cl)nc2c1CN(C(=O)N1CCOC(c3ccncc3)C1)C2)c1cc(N)cc(C(F)(F)F)c1] | 548.29 |
| Example 146. | 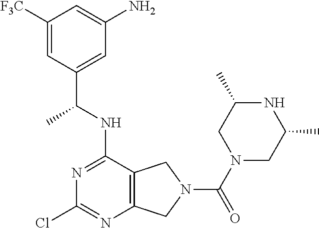 [SMILES: C[C@@H]1CN(C(=O)N2Cc3nc(Cl)nc(N[C@H](C)c4cc(N)cc(C(F)(F)F)c4)c3C2)C[C@H](C)N1] | 498.38 |
| Example 147. | 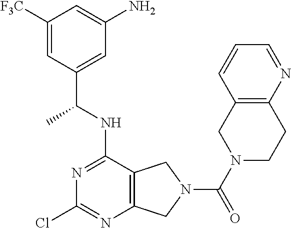 [SMILES: C[C@@H](Nc1nc(Cl)nc2c1CN(C(=O)N1CCc3ncccc3C1)C2)c1cc(N)cc(C(F)(F)F)c1] | 518.38 |
| Example 148. | 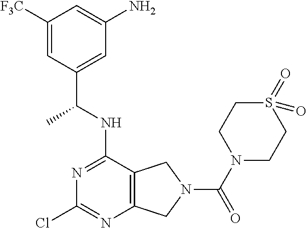 [SMILES: C[C@@H](Nc1nc(Cl)nc2c1CN(C(=O)N1CCS(=O)(=O)CC1)C2)c1cc(N)cc(C(F)(F)F)c1] | 519.28 |
| Example 149. | 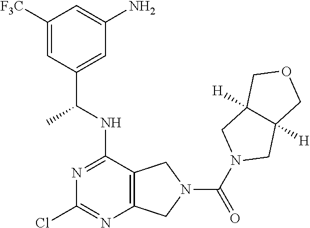 [SMILES: C[C@@H](Nc1nc(Cl)nc2c1CN(C(=O)N1C[C@H]3COC[C@H]3C1)C2)c1cc(N)cc(C(F)(F)F)c1] | 497.38 |
| Example 150. | 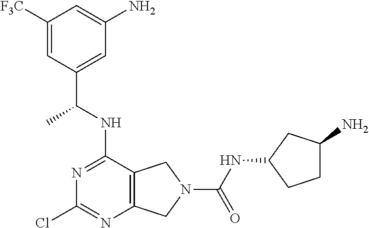 | 484.37 |
| Example 151. | 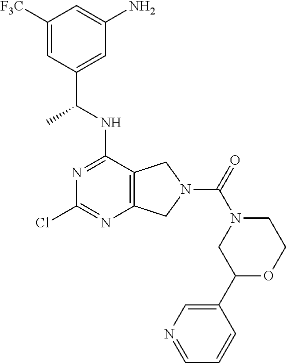 [SMILES: C[C@@H](Nc1nc(Cl)nc2c1CN(C(=O)N1CCOC(c3cccnc3)C1)C2)c1cc(N)cc(C(F)(F)F)c1] | 548.29 |
| Example 152. | 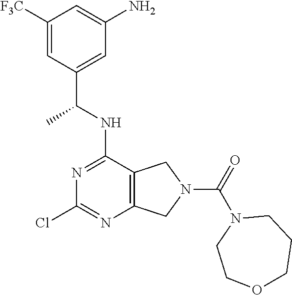 [SMILES: C[C@@H](Nc1nc(Cl)nc2c1CN(C(=O)N1CCCOCC1)C2)c1cc(N)cc(C(F)(F)F)c1] | 444.4 |
| Example 153. | 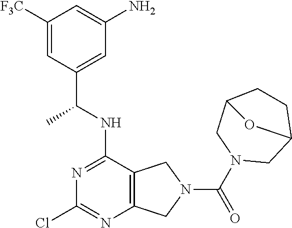 [SMILES: C[C@@H](Nc1nc(Cl)nc2c1CN(C(=O)N1CC3CCC(C1)O3)C2)c1cc(N)cc(C(F)(F)F)c1] | 497.5 |
| Example 154. | 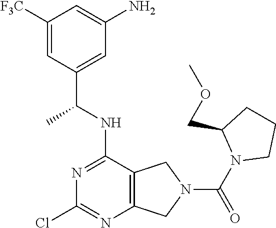 [SMILES: COC[C@H]1CCCN1C(=O)N1Cc2nc(Cl)nc(N[C@H](C)c3cc(N)cc(C(F)(F)F)c3)c2C1] | 499.3, 499.6 |
| Example 155. | 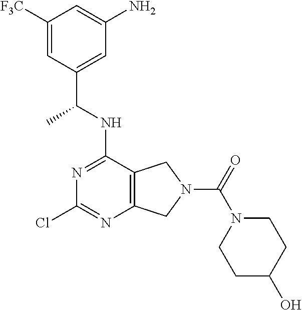 [SMILES: C[C@@H](Nc1nc(Cl)nc2c1CN(C(=O)N1CCC(O)CC1)C2)c1cc(N)cc(C(F)(F)F)c1] | 485.4 |
| Example 156. | 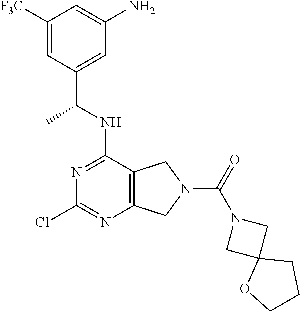 [SMILES: C[C@@H](Nc1nc(Cl)nc2c1CN(C(=O)N1CC3(CCCO3)C1)C2)c1cc(N)cc(C(F)(F)F)c1] | 497.3 |
| Example 157. | 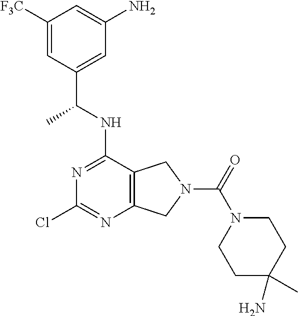 [SMILES: C[C@@H](Nc1nc(Cl)nc2c1CN(C(=O)N1CCC(C)(N)CC1)C2)c1cc(N)cc(C(F)(F)F)c1] | 481.5 |
| Example 158. | 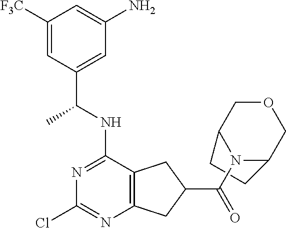 [SMILES: C[C@@H](Nc1nc(Cl)nc2c1CC(C(=O)N1C3CCC1COC3)C2)c1cc(N)cc(C(F)(F)F)c1] | 497.5 |
| Example 159. | 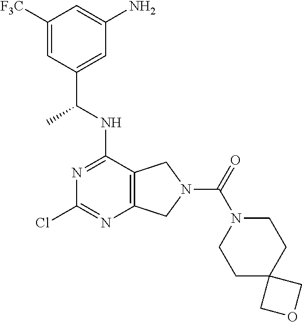 [SMILES: C[C@@H](Nc1nc(Cl)nc2c1CN(C(=O)N1CCC3(CC1)COC3)C2)c1cc(N)cc(C(F)(F)F)c1] | 511.6 |
| Example 160. | 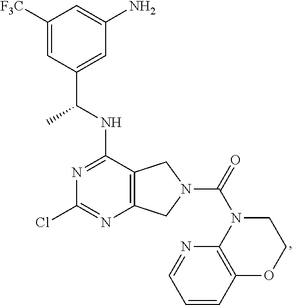 [SMILES: C[C@@H](Nc1nc(Cl)nc2c1CN(C(=O)N1CCOc3cccnc31)C2)c1cc(N)cc(C(F)(F)F)c1] | 520.5 |
| Example 161. | 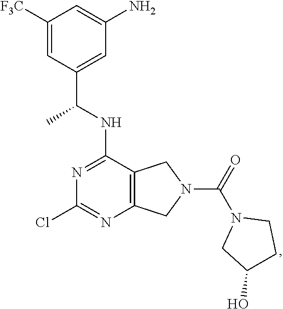 [SMILES: C[C@@H](Nc1nc(Cl)nc2c1CN(C(=O)N1CC[C@H](O)C1)C2)c1cc(N)cc(C(F)(F)F)c1] | 471.5 |
| Example 162. | 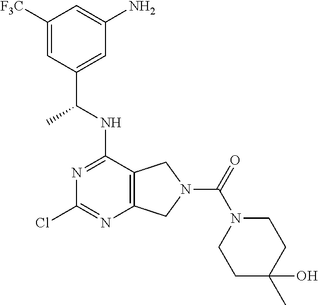 [SMILES: C[C@@H](Nc1nc(Cl)nc2c1CN(C(=O)N1CCC(C)(O)CC1)C2)c1cc(N)cc(C(F)(F)F)c1] | 499.5 |
| Example 163. | 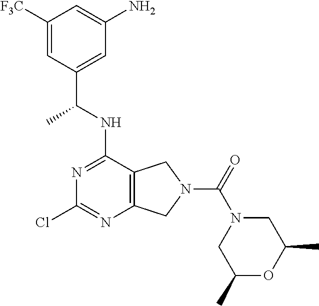 [SMILES: C[C@@H]1CN(C(=O)N2Cc3nc(Cl)nc(N[C@H](C)c4cc(N)cc(C(F)(F)F)c4)c3C2)C[C@H](C)O1] | 499.5 |
| Example 164. | 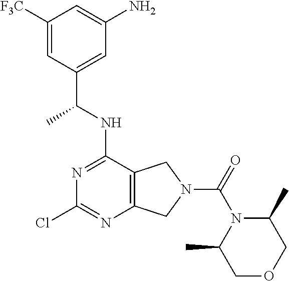 [SMILES: C[C@@H]1COC[C@H](C)N1C(=O)N1Cc2nc(Cl)nc(N[C@H](C)c3cc(N)cc(C(F)(F)F)c3)c2C1] | 501.5 |
| Example 351. | 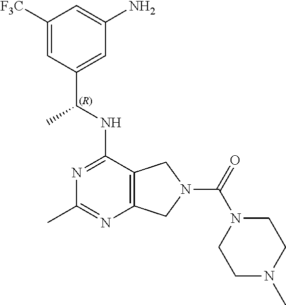 | 464.2 |
| Example 352. | 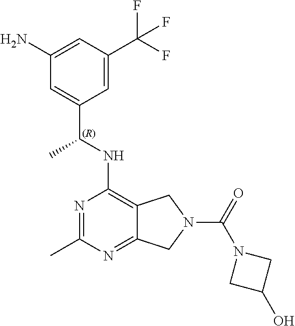 | 437.2 |
| Example 353. | 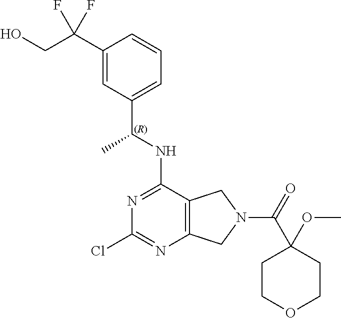 | 497.2 |
| Example 354. | 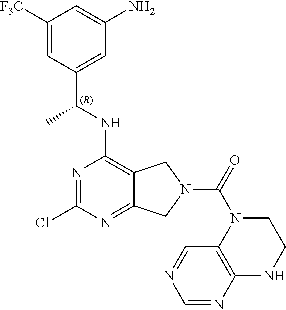 | 520.5 |
| Example 355. | 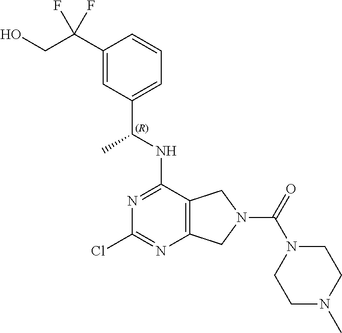 | 481.2 |
| Example 356. | 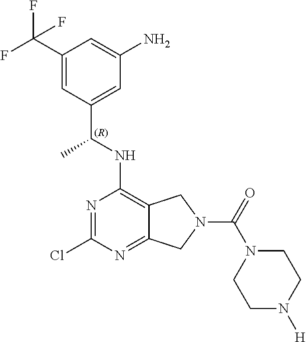 | 470.4 |
| Example 357. | 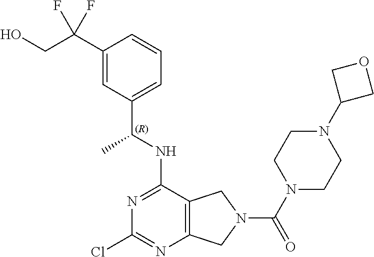 | 523.2 |
| Example 358. | 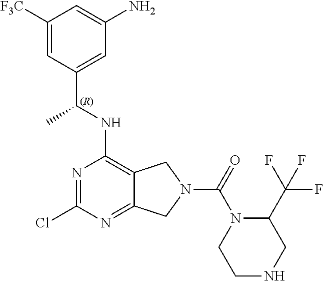 | 538.5 |
| Example 359. | 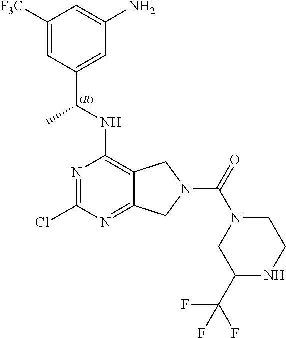 | 538.5 |
| Example 360. | 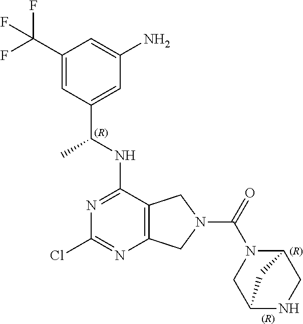 | 482.4 |
| Example 361. | 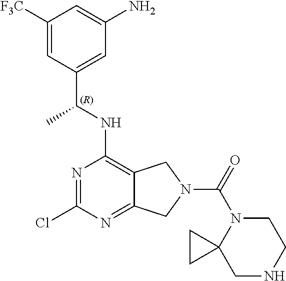 | 496.5 |
| Example 362. | 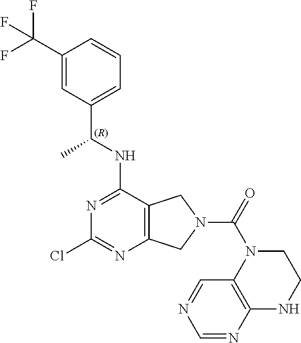 | 505.6 |
| Example 363. | 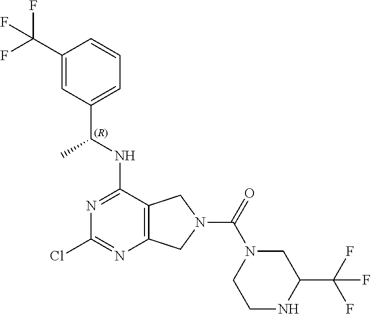 | 523.5 |
| Example 364. | 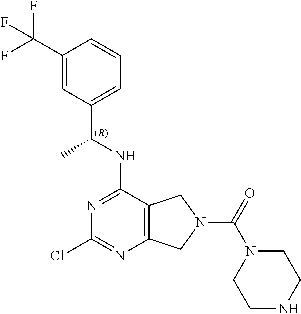 | 455.4 |
| Example 365. | 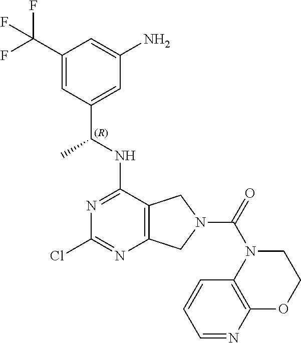 | 520.4 |
| Example 366. | 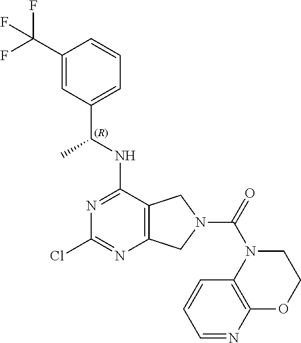 | 505.5 |
| Example 367. | 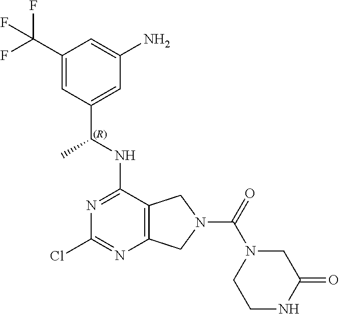 | 484.5 |
| Example 368. | 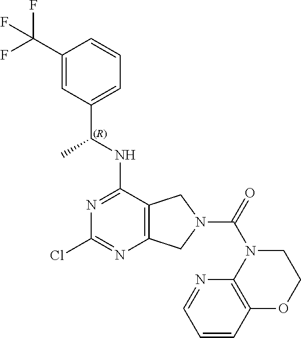 | 505.5 |
| Example 369. | 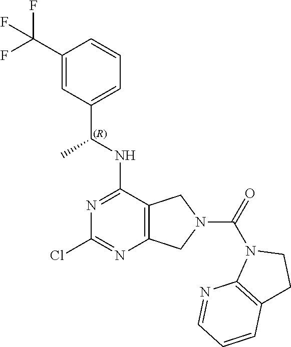 | 489.5 |
| Example 370. | 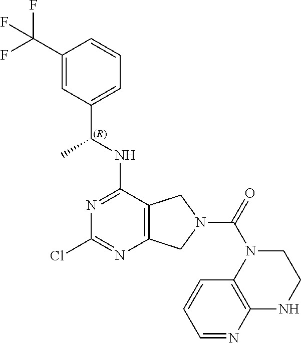 | 504.5 |
| Example 371. | 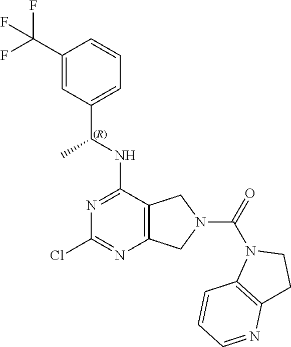 | 489.5 |
| Example 372. | 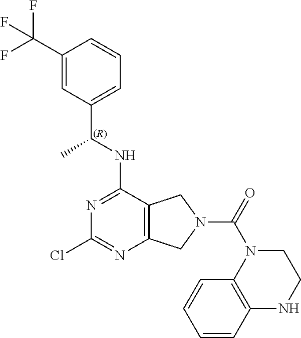 | 503.5 |
| Example 373. | 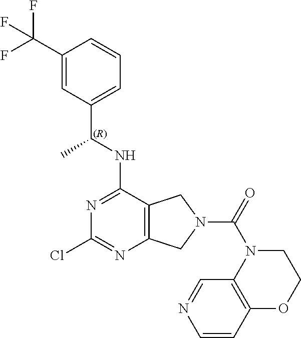 | 505.4 |
| Example 374. | 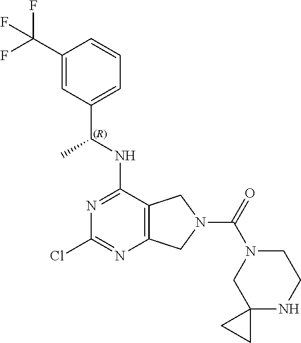 | 481.5 |
| Example 375. | 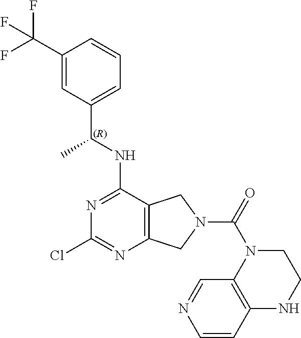 | 504.6 |
| Example 376. | 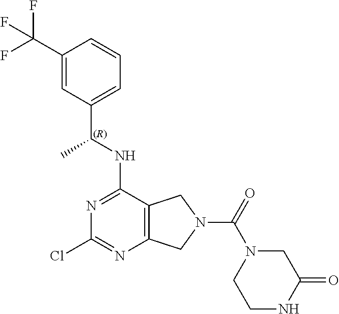 | 469.6 |
| Example 377. | 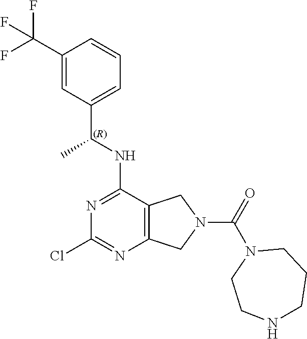 | 469.7 |
| Example 378. | 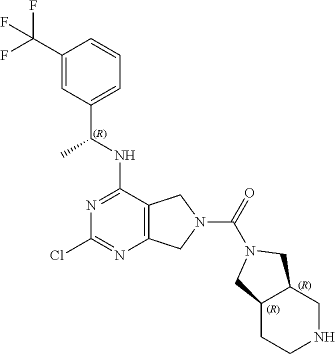 | 495.7 |
| Example 379. | 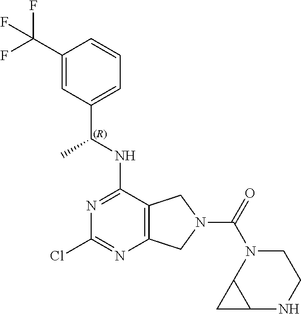 | 467.6 |
| Example 380. | 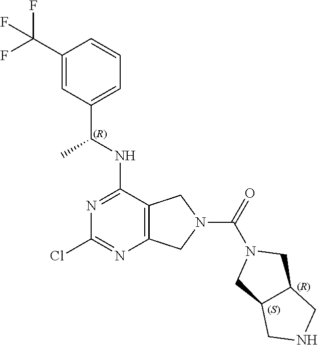 | 481.7 |
| Example 381. | 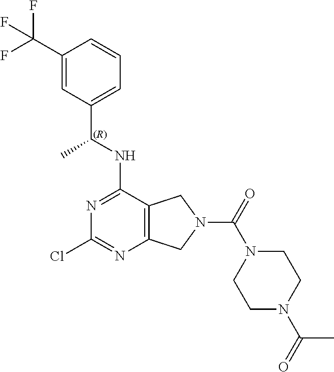 | 497.6 |
| Example 382. | 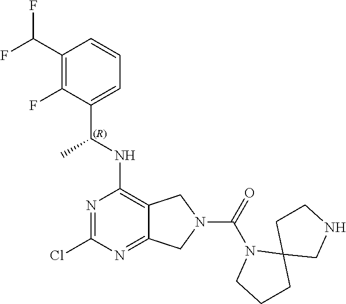 | 495.5 |
| Example 383. | 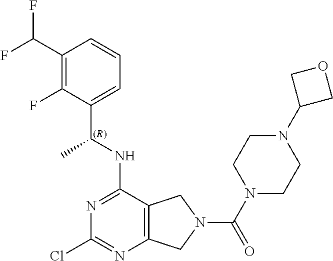 | 511.2 |
| Example 384. | 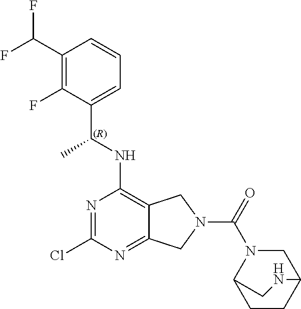 | 481.5 |
| Example 385. | 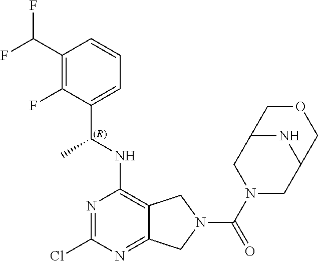 | 497.4 |
| Example 386. | 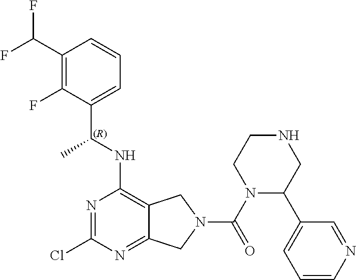 | 532.5 |
| Example 387. | 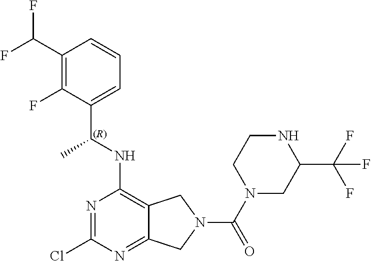 | 523.4 |
| Example 388. | 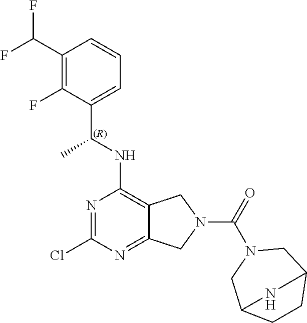 | 481.5 |
| Example 389. | 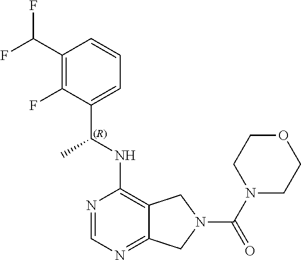 | 422.0 |
| Example 390. | 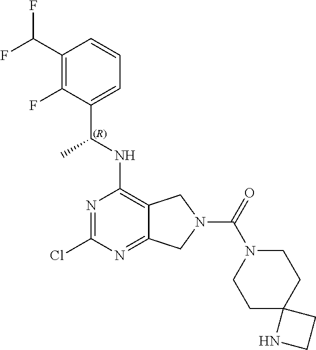 | 495.5 |
| Example 391. | 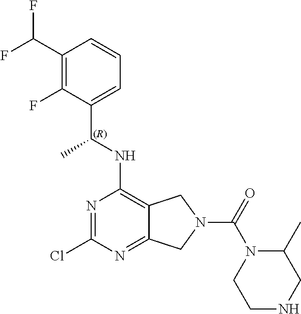 | 469.5 |
| Example 392. | 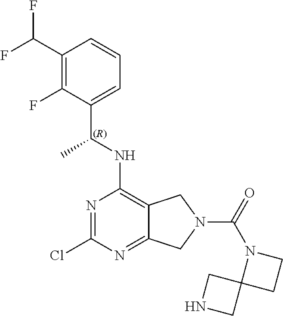 | 467.5 |
| Example 393. | 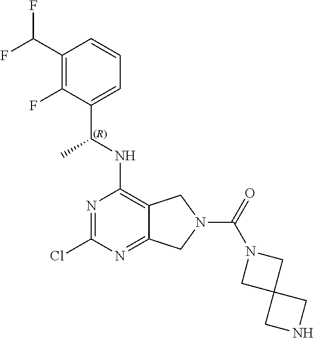 | 467.5 |
| Example 394. | 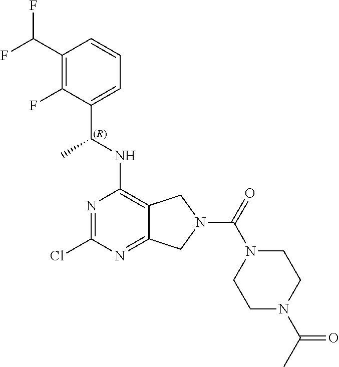 | 497.5 |
| Example 395. | 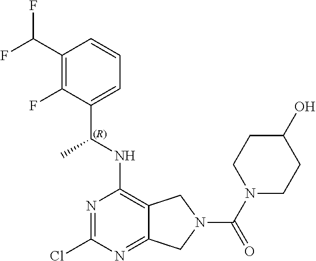 | 470.5 |
| Example 396. | 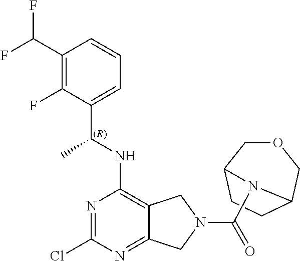 | 482.5 |
| Example 397. | 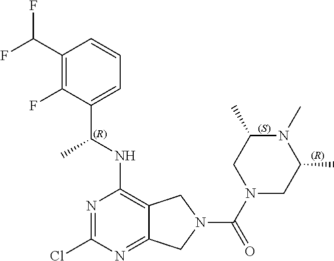 | 497.6 |
| Example 398. | 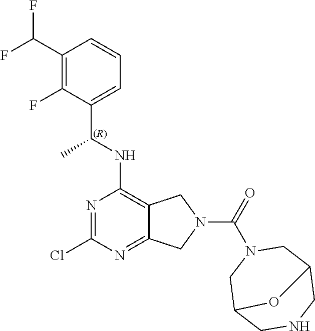 | 497.5 |
| Example 399. | 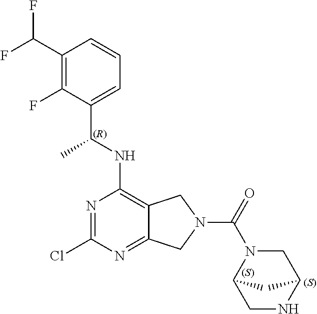 | 467.5 |
| Example 400. | 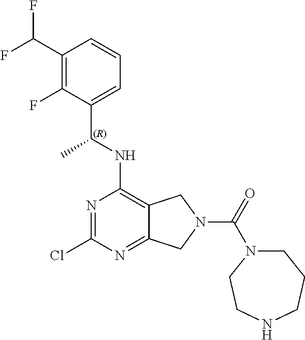 | 469.5 |
| Example 401. | 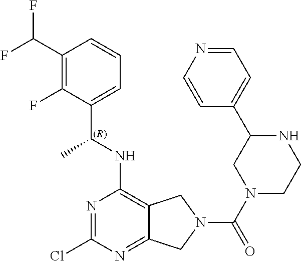 | 532.2 |
| Example 402. | 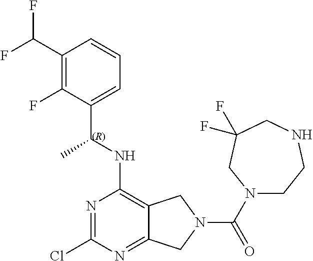 | 505.5 |
| Example 403. | 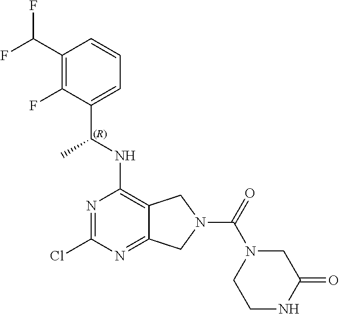 | 469.5 |
| Example 404. | 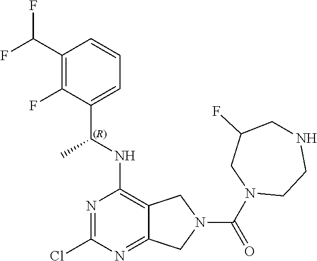 | 487.5 |
| Example 405. | 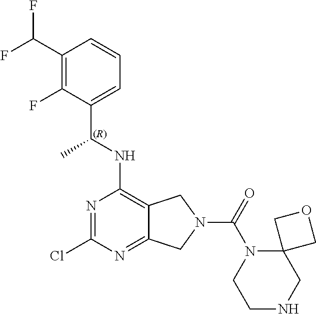 | 497.5 |
| Example 406. | 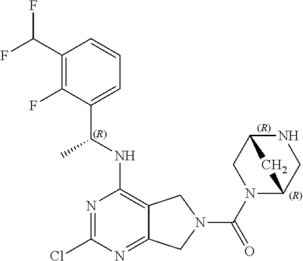 | 467.5 |
| Example 407. | 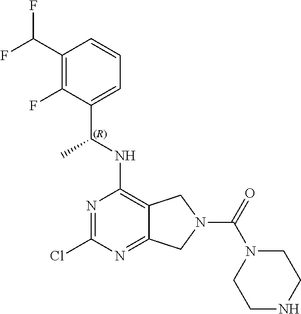 | 455.5 |
| Example 408. | 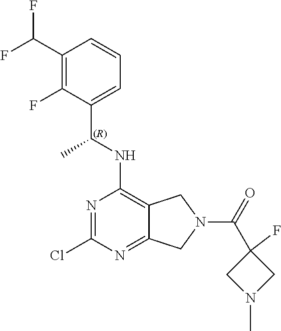 | 458.1 |
| Example 409. | 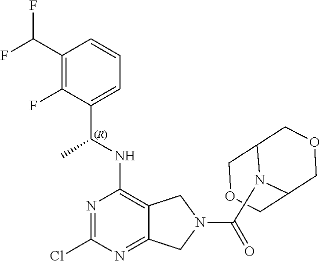 | 498.4 |
| Example 410. | 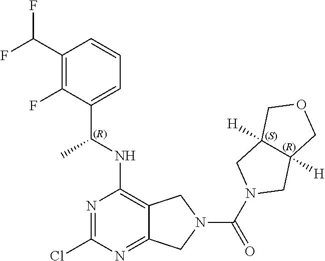 | 482.5 |
| Example 411. | 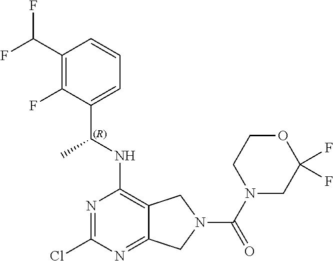 | 492.5 |
| Example 412. | 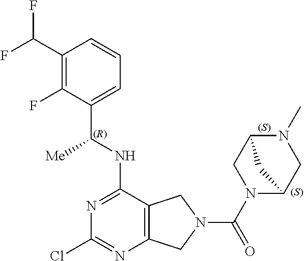 | 481.5 |
| Example 413. | 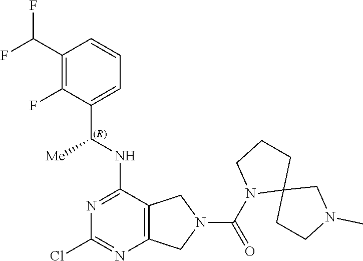 | 509.4 |
| Example 414. | 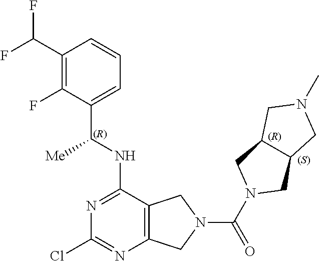 | 495.5 |
| Example 415. | 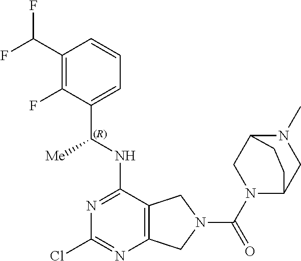 | 495.5 |
| Example 416. | 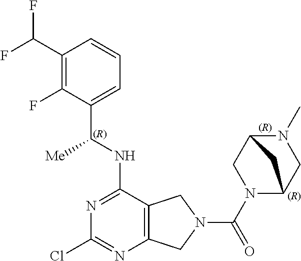 | 481.5 |
| Example 417. | 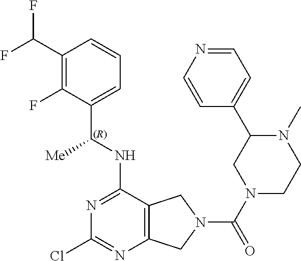 | 546.5 |
| Example 418. | 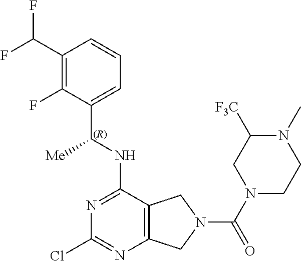 | 537.3 |
| Example 419. | 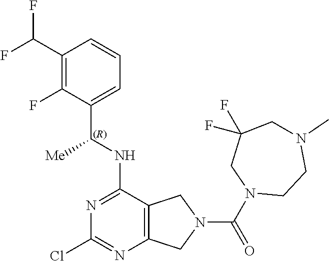 | 519.3 |
| Example 420. | 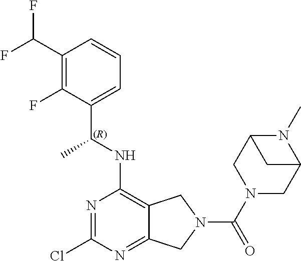 | 481.2 |
| Example 421. | 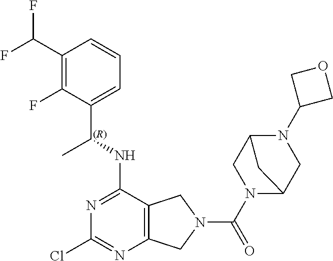 | 523.2 |
| Example 422. | 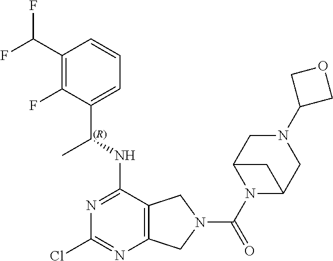 | 523.2 |
| Example 423. | 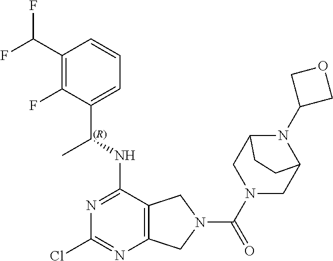 | 537.2 |
| Example 424. | 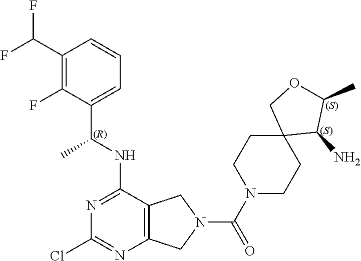 | 539.0 |
| Example 425. | 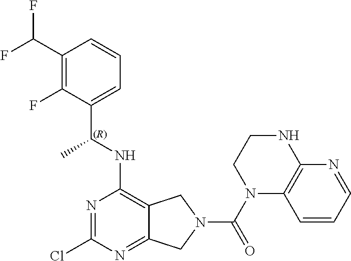 | 503.9 |
| Example 426. | 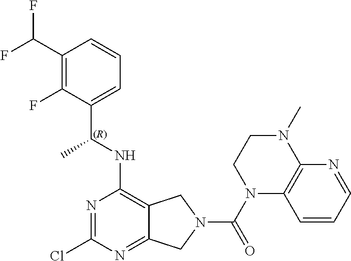 | 518.0 |
| Example 427. | 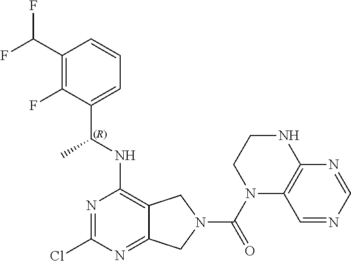 | 504.9 |
| Example 428. | 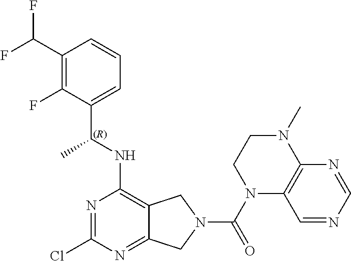 | 519.0 |
| Example 429. | 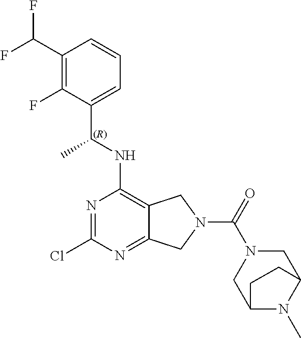 | 495.2 |
| Example 430. | 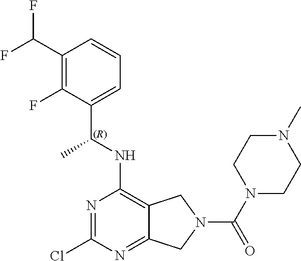 | 469.2 |
| Example 490. | 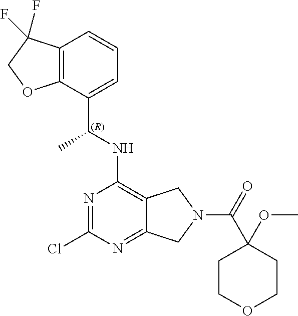 | 495.2 |
| Example 491. | 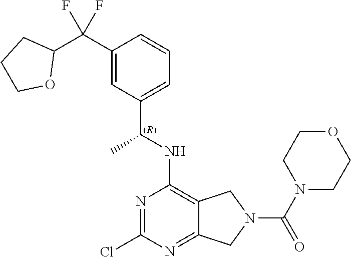 | 508.2 |
| Example 492. | 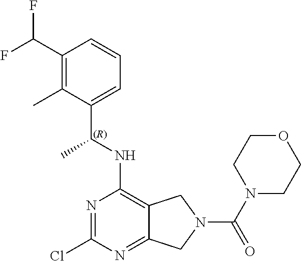 | 452.2 |
| Example 493. | 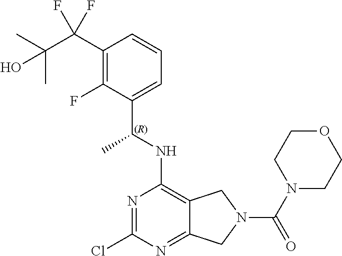 | 514.2 |
| Example 494. | 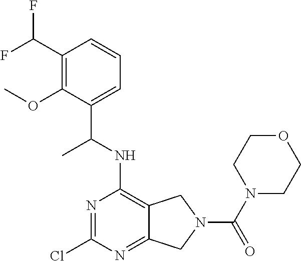 [SMILES: COc1c(C(F)F)cccc1C(C)Nc1nc(Cl)nc2c1CN(C(=O)N1CCOCC1)C2] | 468.1 |
| Example 495. | 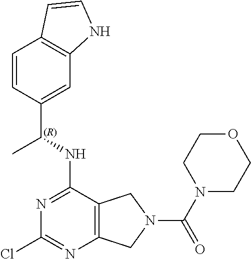 | 427.1 |
| Example 496. | 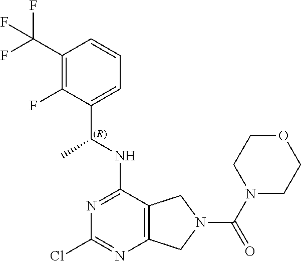 | 474.1 |
| Example 497. | 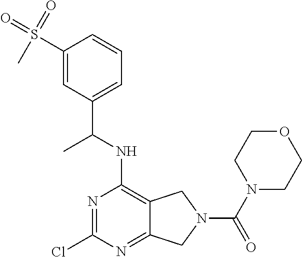 [SMILES: CC(Nc1nc(Cl)nc2c1CN(C(=O)N1CCOCC1)C2)c1cccc(S(C)(=O)=O)c1] | 466.0 |
| Example 498. | 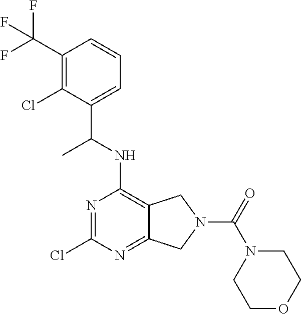 [SMILES: CC(Nc1nc(Cl)nc2c1CN(C(=O)N1CCOCC1)C2)c1cccc(C(F)(F)F)c1Cl] | 490.0 |
| Example 499. | 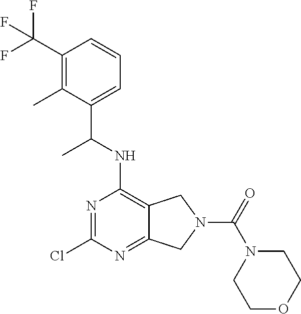 [SMILES: Cc1c(C(C)Nc2nc(Cl)nc3c2CN(C(=O)N2CCOCC2)C3)cccc1C(F)(F)F] | 470.0 |
| Example 537. | 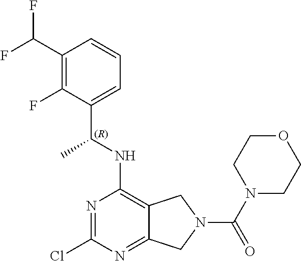 | 455.9 |

### Example 165. Synthesis of N-[(1R)-1-[3-amino-5-(trifluoromethyl)phenyl]ethyl]-2-chloro-6-(morpholine-4-carbonyl)-5,6,7,8-tetrahydroquinazolin-4-amine

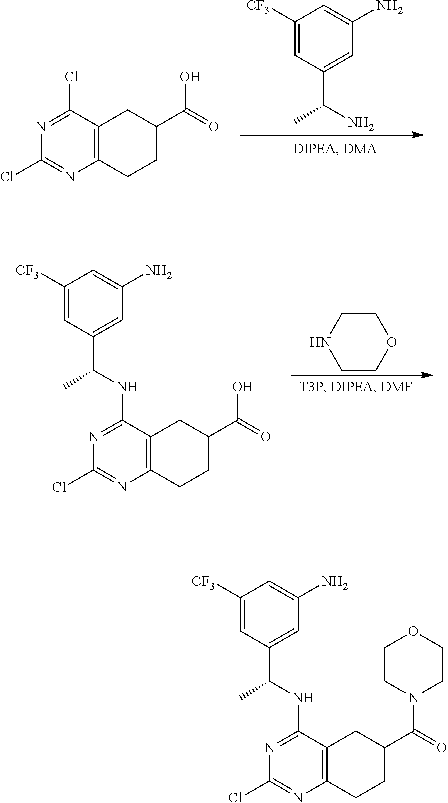 [SMILES: O=C(O)C1CCc2nc(Cl)nc(Cl)c2C1 ; C[C@@H](N)c1cc(N)cc(C(F)(F)F)c1 ; C[C@@H](Nc1nc(Cl)nc2c1CC(C(=O)O)CC2)c1cc(N)cc(C(F)(F)F)c1 ; C1COCCN1 ; C[C@@H](Nc1nc(Cl)nc2c1CC(C(=O)N1CCOCC1)CC2)c1cc(N)cc(C(F)(F)F)c1]

### Step 1.

To a mixture of 3-[(1R)-1-aminoethyl]-5-(trifluoromethyl)aniline (82 mg, 0.40 mmol) and 2,4-dichloro-5,6,7,8-tetrahydroquinazoline-6-carboxylic acid (100 mg, 0.40 mmol) in DMA (1 mL) was added DIPEA (140 μL, 0.81 mmol). The mixture was heated to 40° C. and stirred overnight and the solvent was removed under reduced pressure to give 4-{[(1R)-1-[3-amino-5-(trifluoromethyl)phenyl]ethyl]amino}-2-chloro-5,6,7,8-tetrahydroquinazoline-6-carboxylic acid (167 mg), which was used in the next step without further purification. LCMS (ESI): m/z: [M+H] calculated for C₁₈H₁₉ClF₃N₄O₂: 415.1. found 415.4.

### Step 2.

To a mixture of 4-{[(1R)-1-[3-amino-5-(trifluoromethyl)phenyl]ethyl]amino}-2-chloro-5,6,7,8-tetrahydroquinazoline-6-carboxylic acid (167 mg, 0.40 mmol) and morpholine (35 μL, 0.40 mmol) in DMF (2 mL) was added DIPEA (210 μL, 1.21 mmol) and T3P, 50% wt. in DMF (384 μL, 0.61 mmol). The mixture was stirred at rt for 1 h, then the mixture was diluted with H₂O (at least 25% by volume) and purified by prep-HPLC to give N-[(1R)-1-[3-amino-5-(trifluoromethyl)phenyl]ethyl]-2-chloro-6-(morpholine-4-carbonyl)-5,6,7,8-tetrahydroquinazolin-4-amine (42 mg, 21% yield). LCMS (ESI): m/z: [M+H] calculated for C₂₂H₂₆ClF₃N₅O₂: 484.2. found 484.4; H NMR (500 MHz, METHANOL-d₄) δ ppm 6.94 (s, 1H), 6.90 (dt, J=7.4, 1.9 Hz, 1H), 6.80 (t, J=1.9 Hz, 1H), 5.35 (dt, J=12.2, 6.9 Hz, 1H), 3.67 (dddd, J=23.6, 19.2, 7.8, 5.0 Hz, 8H), 3.15 (m, 1H), 2.82-2.62 (m, 2H), 2.58 (dd, J=8.9, 6.8 Hz, 2H), 2.06-1.94 (m, 1H), 1.84 (dtd, J=13.4, 11.2, 5.5 Hz, 1H), 1.54 (dd, J=7.1, 1.1 Hz, 3H).

### Example 166 (also Example 48). Synthesis of N-[(1R)-1-[3-amino-5-(trifluoromethyl)phenyl]ethyl]-6-(azetidine-3-sulfonyl)-2-chloro-5H,6H,7H-pyrrolo[3,4-d]pyrimidin-4-amine

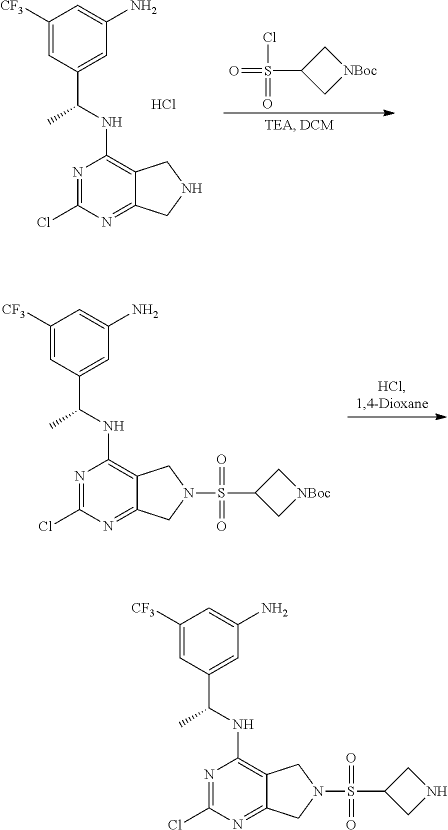 [SMILES: C[C@@H](Nc1nc(Cl)nc2c1CNC2)c1cc(N)cc(C(F)(F)F)c1 ; Cl ; CC(C)(C)OC(=O)N1CC(S(=O)(=O)Cl)C1 ; C[C@@H](Nc1nc(Cl)nc2c1CN(S(=O)(=O)C1CN(C(=O)OC(C)(C)C)C1)C2)c1cc(N)cc(C(F)(F)F)c1 ; C[C@@H](Nc1nc(Cl)nc2c1CN(S(=O)(=O)C1CNC1)C2)c1cc(N)cc(C(F)(F)F)c1]

### Step 1.

To a mixture of N-[(1R)-1-[3-amino-5-(trifluoromethyl)phenyl]ethyl]-2-chloro-5H,6H,7H-pyrrolo[3,4-d]pyrimidin-4-amine HCl salt (200 mg, 0.51 mmol) and TEA (352 μL, 2.53 mmol) in DCM (5 mL) was added tert-butyl 3-(chlorosulfonyl)azetidine-1-carboxylate (129 mg, 0.51 mmol). The mixture was stirred at rt for 1 h and the solvent was removed under reduced pressure to give tert-butyl 3-[(4-{[(1R)-1-[3-amino-5-(trifluoromethyl)phenyl]ethyl]amino}-2-chloro-5H,6H,7H-pyrrolo[3,4-d]pyrimidin-6-yl)sulfonyl]azetidine-1-carboxylate (292 mg), which was used in the next step without further purification. LCMS (ESI): m/z: [M+H] calculated for C₂₃H₂₉ClF₃N₆O₄S: 577.2. found 577.5.

### Step 2.

Tert-butyl 3-[(4-{[(1R)-1-[3-amino-5-(trifluoromethyl)phenyl]ethyl]amino}-2-chloro-5H,6H,7H-pyrrolo[3,4-d]pyrimidin-6-yl)sulfonyl]azetidine-1-carboxylate (292 mg, 0.51 mmol) was dissolved in 4M HCl in 1,4-dioxane/MeOH (1 mL). The mixture was heated to 40° C. and stirred for 1 h, the solvent was removed under reduced pressure and the crude residue was purified by prep-HPLC to give N-[(1R)-1-[3-amino-5-(trifluoromethyl)phenyl]ethyl]-6-(azetidine-3-sulfonyl)-2-chloro-5H,6H,7H-pyrrolo[3,4-d]pyrimidin-4-amine (29 mg, 12% yield). LCMS (ESI): m/z: [M+H] calculated for C₁₈H₂₁ClF₃N₆O₂S: 477.1. found 477.4; ¹H NMR (500 MHz, METHANOL-d₄) δ ppm 6.93-6.87 (m, 2H), 6.83-6.79 (m, 1H), 5.32 (d, J=7.4 Hz, 1H), 4.63-4.54 (m, 3H), 4.53-4.49 (m, 2H), 4.27 (d, J=7.7 Hz, 4H), 1.53 (d, J=7.0 Hz, 3H).

### Example 167. Synthesis of [2-chloro-4-[[(1R)-1-[3-(1,1-difluoro-2-hydroxy-ethyl)phenyl]ethyl]amino]-5,7-dihydropyrrolo[3,4-d]pyrimidin-6-yl]-(4-methoxytetrahydropyran-4-yl)methanone

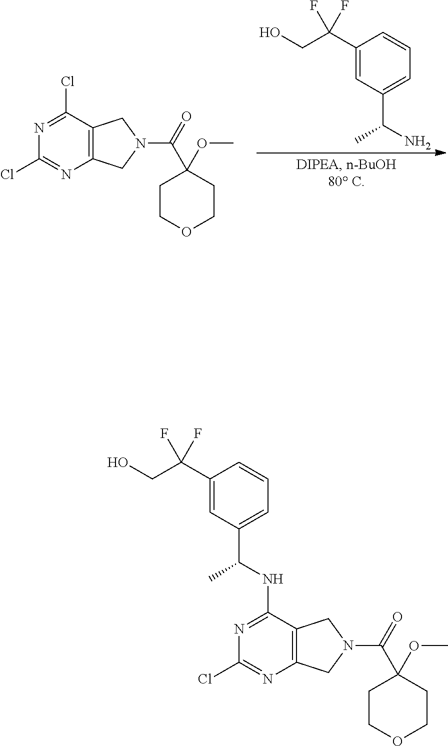 [SMILES: COC1(C(=O)N2Cc3nc(Cl)nc(Cl)c3C2)CCOCC1 ; C[C@@H](N)c1cccc(C(F)(F)CO)c1 ; COC1(C(=O)N2Cc3nc(Cl)nc(N[C@H](C)c4cccc(C(F)(F)CO)c4)c3C2)CCOCC1]

### Step 1.

A mixture of 2-[3-[(1R)-1-aminoethyl]phenyl]-2,2-difluoro-ethanol (100 mg, 0.5 mmol), (2,4-dichloro-5,7-dihydropyrrolo[3,4-d]pyrimidin-6-yl)-(4-methoxytetrahydropyran-4-yl)methanone (165 mg, 0.5 mmol) and DIPEA (260 μL, 1.49 mmol) in n-BuOH (3 mL) was heated to 80° C. and stirred for 5 h. The mixture was filtered and the filtrate was purified by prep-HPLC to give [2-chloro-4-[[(1R)-1-[3-(1,1-difluoro-2-hydroxy-ethyl)phenyl]ethyl]amino]-5,7-dihydropyrrolo[3,4-d]pyrimidin-6-yl]-(4-methoxytetrahydropyran-4-yl)methanone (35 mg, 14% yield). LCMS (ESI): m/z: [M+H] calculated for C₂₃H₂₈ClF₂N₄O₄: 497.18. found 497.1; H NMR (400 MHz, DMSO-d₆) δ ppm 8.33 (br dd, J=11.5, 8.1, 1H), 7.59 (s, 1H), 7.55-7.51 (m, 1H), 7.46 (dt, J=7.5, 3.8 Hz, 1H), 7.43-7.38 (m, 1H), 5.64 (t, J=6.3 Hz, 1H), 5.35 (q, J=6.9 Hz, 1H), 4.89-4.76 (m, 2H), 4.60-4.46 (m, 2H), 3.85 (dt, J=14.4, 6.4 Hz, 2H), 3.19 (s, 3H), 3.21-3.12 (m, 3H), 1.97-1.84 (m, 4H), 1.51 (t, J=6.8 Hz, 3H).

### Example 168. Synthesis of [2-chloro-4-[[(1R)-1-[3-(1,1-difluoro-2-hydroxy-ethyl)phenyl]ethyl]amino]-5,7-dihydropyrrolo[3,4-d]pyrimidin-6-yl]-morpholino-methanone

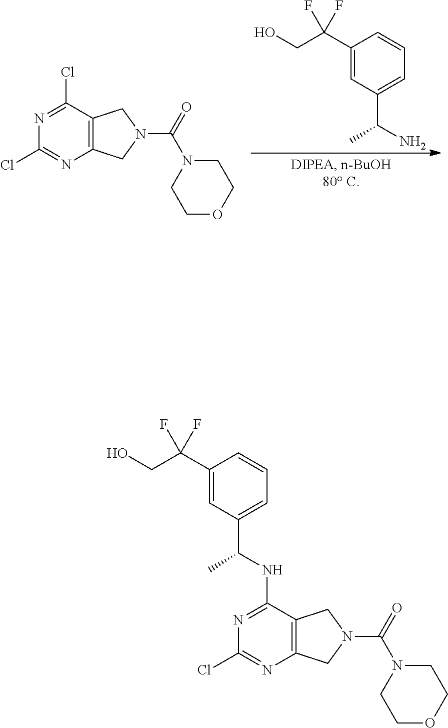 [SMILES: O=C(N1CCOCC1)N1Cc2nc(Cl)nc(Cl)c2C1 ; C[C@@H](N)c1cccc(C(F)(F)CO)c1 ; C[C@@H](Nc1nc(Cl)nc2c1CN(C(=O)N1CCOCC1)C2)c1cccc(C(F)(F)CO)c1]

A mixture of 2-[3-[(1R)-1-aminoethyl]phenyl]-2,2-difluoro-ethanol (100 mg, 0.5 mmol), (2,4-dichloro-5,7-dihydropyrrolo[3,4-d]pyrimidin-6-yl)-morpholino-methanone (151 mg, 0.5 mmol) and DIPEA (260 μL, 1.49 mmol) in n-BuOH (3 mL) was heated to 80° C. and stirred for 5 h. The mixture was filtered and the filtrate was purified by prep-HPLC to give [2-chloro-4-[[(1R)-1-[3-(1,1-difluoro-2-hydroxy-ethyl)phenyl]ethyl]amino]-5,7-dihydropyrrolo[3,4-d]pyrimidin-6-yl]-morpholino-methanone (35 mg, 15% yield). LCMS (ESI): m/z: [M+H] calculated for C₂₁H₂₅ClF₂N₅O₃: 468.16. found 468.1; ¹H NMR (400 MHz, DMSO-d₆) δ ppm 8.17 (br d, J=8.0 Hz, 1H), 7.55 (s, 1H), 7.50 (d, J=7.6 Hz, 1H), 7.44 (t, J=7.6 Hz, 1H), 7.38 (d, J=7.6 Hz, 1H), 5.62 (t, J=6.4 Hz, 1H), 5.33 (m, 1H), 4.61-4.43 (m, 4H), 3.83 (dt, J=14.2, 6.4 Hz, 2H), 3.67-3.59 (m, 4H), 3.28-3.20 (m, 4H), 1.50 (d, J=7.0 Hz, 3H).

### Examples 169. and 170. Synthesis of N-[(1R)-1-[3-amino-5-(trifluoromethyl)phenyl]ethyl]-2-chloro-6-morpholino-5,6,7,8-tetrahydroquinazolin-4-amine

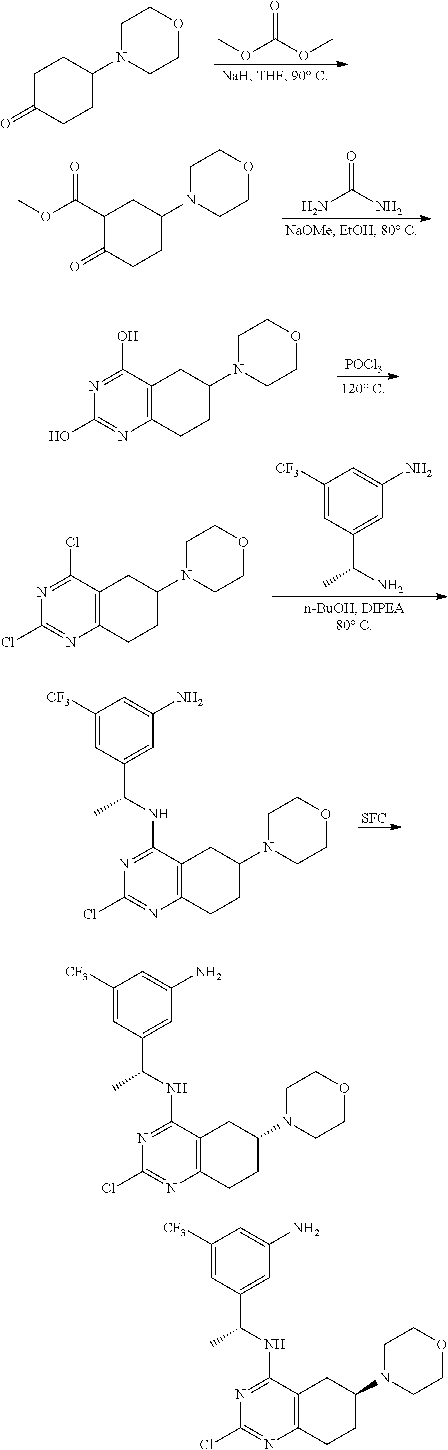

### Step 1.

To a mixture of 4-morpholinocyclohexanone (4.0 g, 21.8 mmol) and dimethyl carbonate (40 mL, 475 mmol) was added NaH, 60% dispersion in oil (1.75 g, 43.7 mmol). The mixture was heated to reflux and stirred for 2 h, then H₂O (50 mL) added and the mixture acidified with aqueous HCl to pH −5. The mixture was extracted with EtOAc (50 mL×5), dried over Na₂SO₄, filtered and the solvent was concentrated under reduced pressure to give methyl 5-morpholino-2-oxo-cyclohexanecarboxylate (4 g, 76% yield). LCMS (ESI): m/z: [M+H] calculated for C₁₂H₂₀NO₄: 242.13. found 242.1.

### Step 2.

To a mixture of methyl 5-morpholino-2-oxo-cyclohexanecarboxylate (4.0 g, 16.6 mmol) and urea (4.44 mL, 82.9 mmol) in EtOH (100 mL) was added NaOMe (4.48 g, 82.9 mmol). The mixture was heated to 80° C. and stirred for 16 h, then H₂O (50 mL) added and the and the mixture acidified with aqueous HCl to pH −5. The solvent was concentrated under reduced pressure and the residue was triturated with EtOAc (40 mL) at 50° C. for 30 min. The mixture was filtered and the residue was dried under vacuum to give 6-morpholino-5,6,7,8-tetrahydroquinazoline-2,4-diol (4 g). LCMS (ESI): m/z: [M+H] calculated for C₁₂H₁₈N₃O₃: 252.13. found 252.1.

### Step 3.

A mixture of 6-morpholino-5,6,7,8-tetrahydroquinazoline-2,4-diol (1.0 g, 4 mmol) and POCl₃ (10 mL) was heated to 100° C. and stirred for 1 h. The solvent was concentrated under reduced pressure, H₂O (20 mL) was added and the pH adjusted to −8 with aqueous NaHCO₃. The mixture was extracted with EtOAc (30 mL×4), the combined organic layers were dried over Na₂SO₄, filtered and the solvent concentrated under reduced pressure to give 4-(2,4-dichloro-5,6,7,8-tetrahydroquinazolin-6-yl)morpholine (230 mg, 17% yield; Note: 85% purity). LCMS (ESI): m/z: [M+H] calculated for C₁₂H₁₆Cl₂N₃O: 288.06. found 288.0.

### Step 4.

To a mixture of 4-(2,4-dichloro-5,6,7,8-tetrahydroquinazolin-6-yl)morpholine (200 mg, 0.69 mmol) and 3-[(1R)-1-aminoethyl]-5-(trifluoromethyl)aniline (213 mg, 1.04 mmol) in t-BuOH (2 mL) was added DIPEA (604 μL, 3.47 mmol). The mixture was heated to 90° C. and stirred for 10 h. The mixture was concentrated under reduced pressure, H₂O (20 mL) added and the mixture extracted with EtOAc (20 mL×4). The combined organic layers were dried over Na₂SO₄, filtered, the solvent was concentrated under reduced pressure and the residue was purified by prep-HPLC to give N-[(1R)-1-[3-amino-5-(trifluoromethyl)phenyl]ethyl]-2-chloro-6-morpholino-5,6,7,8-tetrahydroquinazolin-4-amine (50 mg, 15% yield). LCMS (ESI): m/z: [M+H] calculated for C₂₁H₂₆ClF₃N₅O: 456.17. found 456.2; ¹H NMR (400 MHz, METHANOL-d₄) δ ppm 6.91-6.94 (m, 2H), 6.81 (s, 1H), 5.34-5.40 (m, 1H), 3.78 (t, J=3.6 Hz, 4H), 2.82 (d, J=4.4 Hz, 5H), 2.69-2.73 (m, 3H), 2.44-2.33 (m, 1H), 2.21 (d, J=12.4 Hz, 1H), 1.68-1.70 (m, 1H), 1.56 (d, J=7.2 Hz, 3H).

### Step 5.

N-[(1R)-1-[3-amino-5-(trifluoromethyl)phenyl]ethyl]-2-chloro-6-morpholino-5,6,7,8-tetrahydroquinazolin-4-amine was separated by SFC to give each enantiomer (60 mg and 52 mg). Enantiomer 1: LCMS (ESI): m/z: [M+H] calculated for C₂₁H₂₆ClF₃N₅O: 456.17. found 456.0; ¹H NMR (400 MHz, METHANOL-d₄) δ ppm 6.91-6.94 (m, 2H), 6.80 (s, 1H), 5.34-5.40 (m, 1H), 3.75 (t, J=3.6 Hz, 4H), 2.69-2.76 (m, 8H), 2.36-2.37 (m, 1H), 2.21 (d, J=12.4 Hz, 1H), 1.56-1.64 (m, 1H), 1.54 (d, J=7.2 Hz, 3H). Enantiomer 2: LCMS (ESI): m/z: [M+H] calculated for C₂₁H₂₆ClF₃N₅O: 456.17. found 456.0; ¹H NMR (400 MHz, METHANOL-d₄) δ ppm 6.90-6.93 (m, 2H), 6.80 (s, 1H), 5.33-5.36 (m, 1H), 3.75 (t, J=3.6 Hz, 4H), 2.66-2.70 (m, 8H), 2.31-2.38 (m, 1H), 2.20 (d, J=12.4 Hz, 1H), 1.56-1.63 (m, 1H), 1.55 (d, J=7.2 Hz, 3H).

The following examples 431-444 shown in Table 6 were synthesized in the manner similar to Examples 169 and 170.

TABLE 6
Examples 431-444

| Example # | Structure | Mass Found |
|---|---|---|
| Example 431. | 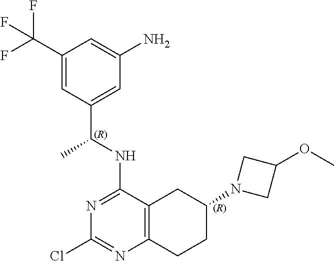 | 456.2 |
| Example 432. | 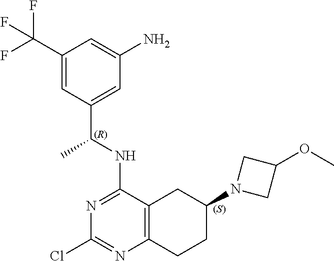 | 456.2 |
| Example 433. | 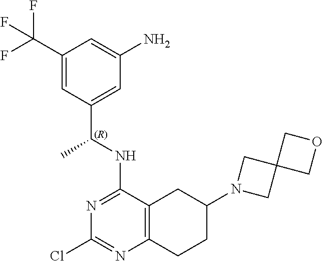 | 468.0 |
| Example 434. | 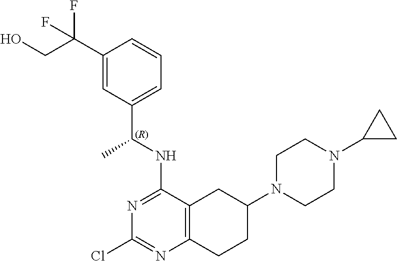 | 492.1 |
| Example 435. | 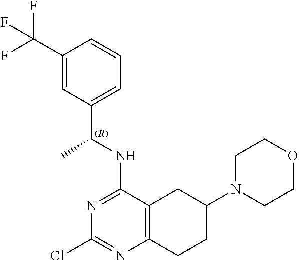 | 441.0 |
| Example 436. | 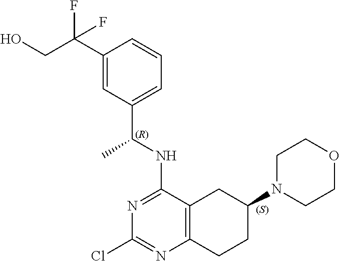 | 453.3 |
| Example 437. | 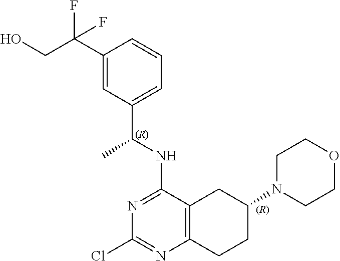 | 453.3 |
| Example 438. | 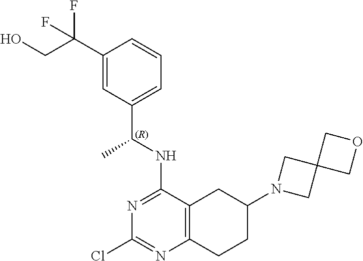 | 465.2 |
| Example 439. | 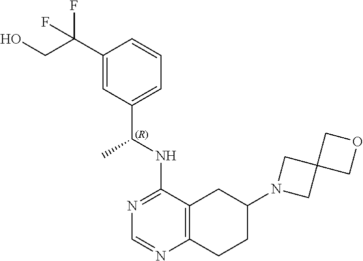 | 431.2 |
| Example 440. | 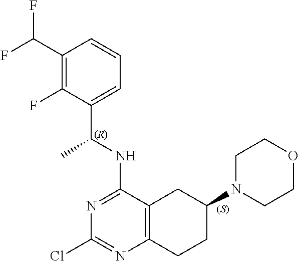 | 441.2 |
| Example 441. | 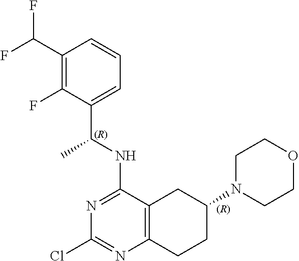 | 441.2 |
| Example 442. | 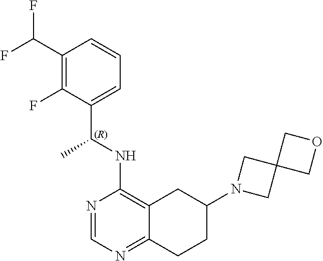 | 419.2 |
| Example 443. | 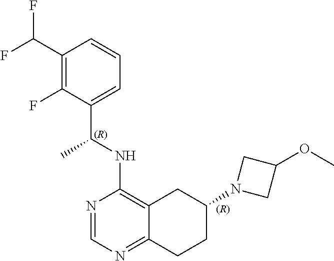 | 407.2 |
| Example 444. | 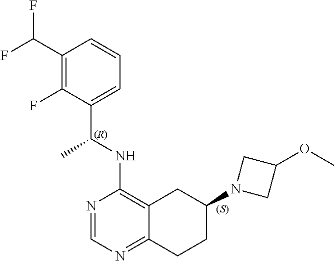 | 407.2 |

### Example 171. Synthesis of [2-chloro-4-[1-[3-(1,1-difluoro-2-hydroxy-ethyl)phenyl]ethylamino]-5,7-dihydropyrrolo[3,4-d]pyrimidin-6-yl]-morpholino-methanone

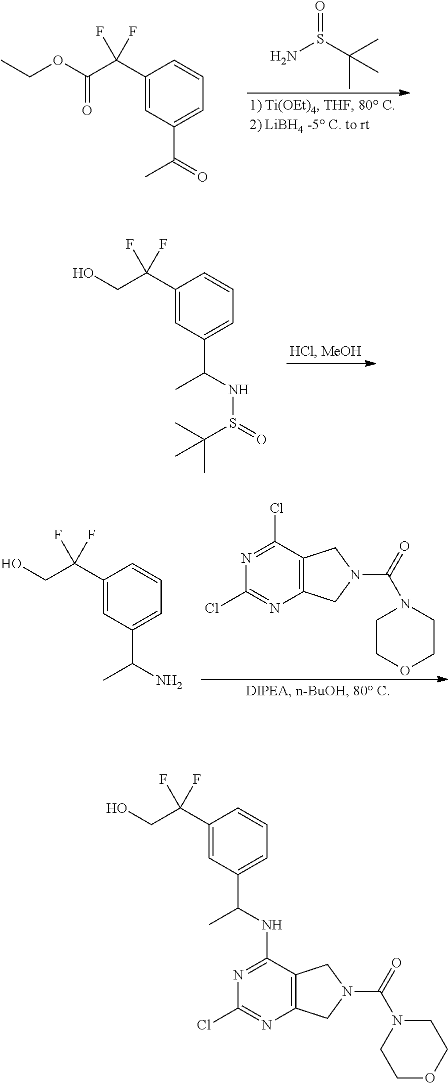

### Step 1.

To a mixture of 2-methylpropane-2-sulfinamide (400 mg, 3.3 mmol) and ethyl 2-(3-acetylphenyl)-2,2-difluoro-acetate (400 mg, 1.65 mmol) in THF (5 mL) at rt was added Ti(OEt)₄ (1.03 mL, 4.95 mmol). The mixture was heated to 80° C. and stirred for 11 h. The mixture was cooled to −5° C., MeOH (67 μL, 1.65 mmol) was added and LiBH₄ (36 mg, 1.65 mmol) was added. The mixture was stirred at rt for 1 h, then cooled to 0° C., H₂O (10 mL) added and extracted with EtOAc (15 mL×3). The combined organic layers were washed with brine (30 mL), dried over anhydrous Na₂SO₄, and filtered. The solvent was concentrated under reduced pressure and the residue was purified by N-[1-[3-(1,1-difluoro-2-hydroxy-ethyl)phenyl]ethyl]-2-methyl-propane-2-sulfinamide (90 mg, 18% yield). LCMS (ESI): m/z: [M+H] calculated for Cl₄H22F₂NO₂S: 306.13. found 306.0; H NMR (400 MHz, CDCl₃) δ ppm 7.55 (s, 1H), 7.49-7.42 (m, 3H), 4.56 (m, 1H), 3.97 (dt, J=12.4, 3.6 Hz, 2H), 3.47 (d, J=3.6 Hz, 1H), 2.81 (br s, 1H), 1.55 (d, J=6.4 Hz, 3H), 1.23 (s, 9H).

### Step 2.

To a mixture of N-[1-[3-(1,1-difluoro-2-hydroxy-ethyl)phenyl]ethyl]-2-methyl-propane-2-sulfinamide (90 mg, 0.29 mmol) in MeOH (2 mL) was added 4M HCl in MeOH (147 μL, 0.58 mmol). The mixture was stirred at rt for 12 h, then neutralized to pH ˜7 with the addition of NaOH in MeOH, filtered and the filtrate concentrated under reduced pressure. The residue was washed with DCM/MeOH (5:1) and the combined organic layers were concentrated under reduced pressure to give 2-[3-(1-aminoethyl)phenyl]-2,2-difluoro-ethanol (55 mg, 93% yield). LCMS (ESI): m/z: [M+H] calculated for C₁₀H₁₄F₂NO: 202.10. found 202.1; ¹H NMR (400 MHz, DMSO-d₆) δ ppm 8.46 (br s, 2H), 7.69 (s, 1H), 7.64 (d, J=6.4 Hz, 1H), 7.59-7.50 (m, 2H), 5.69 (t, J=6.0 Hz, 1H), 4.48 (q, J=6.8 Hz, 1H), 3.87 (dt, J=14.2, 6.0 Hz, 2H), 1.51 (d, J=6.8 Hz, 3H).

### Step 3.

A mixture of 2-[3-(1-aminoethyl)phenyl]-2,2-difluoro-ethanol (55 mg, 0.27 mmol), (2,4-dichloro-5,7-dihydropyrrolo[3,4-d]pyrimidin-6-yl)-morpholino-methanone (83 mg, 0.27 mmol) and DIPEA (143 μL, 0.82 mmol) in n-BuOH (2 mL) was heated to 80° C. and stirred for 24 h. The mixture was filtered and the filtrate was purified by prep-HPLC (×2) to give [2-chloro-4-[1-[3-(1,1-difluoro-2-hydroxy-ethyl)phenyl]ethylamino]-5,7-dihydropyrrolo[3,4-d]pyrimidin-6-yl]-morpholino-methanone (18 mg, 14% yield). LCMS (ESI): m/z: [M+H] calculated for C₂₁H₂₅ClF₂N₅O₃: 468.16. found 468.1; ¹H NMR (400 MHz, METHANOL-d₄) δ ppm 7.59 (s, 1H), 7.51 (d, J=6.6 Hz, 1H), 7.46-7.39 (m, 2H), 5.43 (q, J=6.8 Hz, 1H), 4.62-4.60 (m, 2H), 4.57-4.55 (m, 2H), 3.88 (dt, J=13.2, 1.6 Hz, 2H), 3.75-3.69 (m, 4H), 3.38-3.33 (m, 4H), 1.59 (d, J=7.2 Hz, 3H).

### Example 176. Synthesis of (R)-5-((1-(3-amino-5-(trifluoromethyl)phenyl)ethyl)amino)-1-methyl-3-(tetrahydro-2H-pyran-4-yl)-3,4-dihydropyrimido[4,5-d]pyrimidin-2(1H)-one

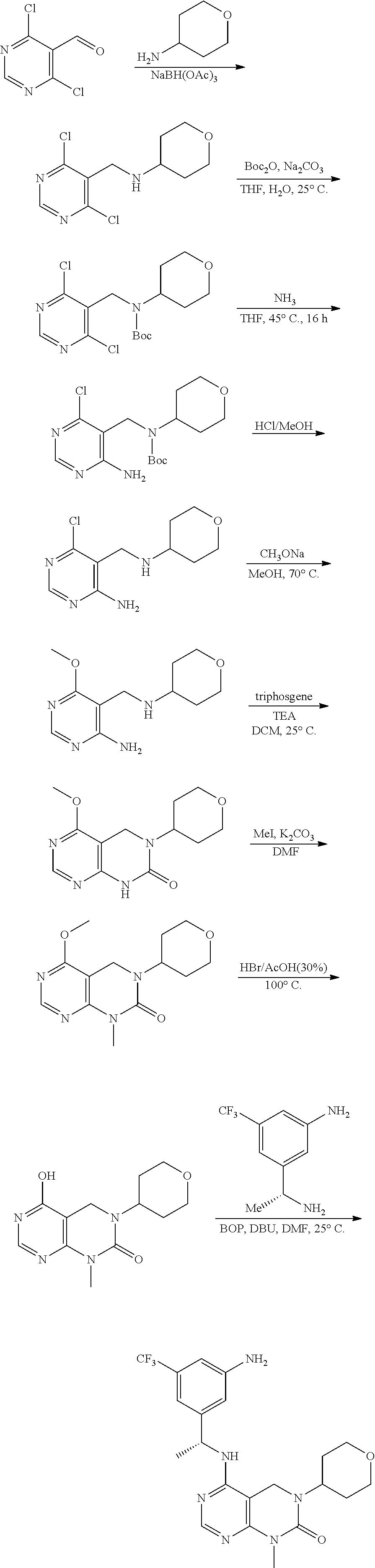

### Step 1.

To a solution of 4,6-dichloropyrimidine-5-carbaldehyde (7.5 g, 42 mmol) in DCE (80 mL) and HOAc (8 mL) was added tetrahydropyran-4-amine (6.4 g, 64 mmol) and NaBH(OAc)₃ (27 g, 127 mmol) at 0° C. The reaction was stirred at 25° C. for 2 h and then diluted with H₂O. The mixture was used into the next step without further purification. LCMS (ESI): m/z: [M+H] calculated for C₁₀H₁₃Cl₂N₃O: 262.0. found 262.0.

### Step 2.

To a solution of N-[(4,6-dichloropyrimidin-5-yl)methyl]tetrahydropyran-4-amine (12 g, 46 mmol) in H₂O (100 mL) and THF (100 mL) was added (Boc)₂O (30 g, 137 mmol) and Na₂CO₃ (9.7 g, 92 mmol). The reaction was stirred at 25° C. for 12 h and then diluted with H₂O. The mixture was extracted with EtOAc and the combined organic layers were washed with brine, dried with Na₂SO₄ and the solvent was removed under reduced pressure. The residue was purified by silica gel column to give tert-butyl N-[(4,6-dichloropyrimidin-5-yl)methyl]-N-tetrahydropyran-4-yl-carbamate (13 g, 78% yield). LCMS (ESI): m/z: [M+H] calculated for C₁₅H₂₁Cl₂N₃O₃: 362.1. found 362.3.

### Step 3.

A solution of tert-butyl N-[(4,6-dichloropyrimidin-5-yl)methyl]-N-tetrahydropyran-4-yl-carbamate (13 g, 36 mmol) in THF (40 mL) and NH₃ (30% in H₂O, 80 mL) was stirred at 45° C. for 16 h. After cooling to rt water was added and the mixture was extracted with EtOAc. The combined organic layers were washed with brine and dried with Na₂SO₄. The solvent was removed under reduced pressure and the residue was purified by column chromatography to give tert-butyl N-[(4-amino-6-chloro-pyrimidin-5-yl)methyl]-N-tetrahydropyran-4-yl-carbamate (4.1 g, 33% yield). ¹H NMR (400 MHz, CHLOROFORM-d) δ=8.24 (s, 1H), 4.62 (s, 2H), 4.00-3.96 (m, 2H), 3.49-3.47 (m, 1H), 3.33-3.27 (m, 2H), 2.20-2.11 (m, 2H), 1.61 (s, 1H), 1.52 (s, 9H), 1.48-1.44 (m, 2H).

### Step 4.

A solution of tert-butyl N-[(4-amino-6-chloro-pyrimidin-5-yl)methyl]-N-tetrahydropyran-4-yl-carbamate (4.1 g, 12 mmol) in HCl/MeOH (4 M, 60 ml) was stirred at 25° C. for 2 h. The solvent was removed under reduced pressure and the residue was diluted with MeOH. The solution was adjusted to pH ˜12 by the addition of MeOH/NaOH to pH ˜12. The solvent was removed under reduced pressure to give 6-chloro-5-[(tetrahydropyran-4-ylamino) methyl]pyrimidin-4-amine (4.1 g, crude). LCMS (ESI): m/z: [M+H] calculated for C₁₁H₁₉N₄O₂: 239.1. found 239.1.

### Step 5.

To a solution of 6-chloro-5-[(tetrahydropyran-4-ylamino)methyl]pyrimidin-4-amine (4.1 g, 17 mmol) in MeOH (60 mL) was added CH₃ONa (4.56 g, 84.5 mmol). The mixture was stirred at 70° C. for 3 h. The solvent was removed under reduced pressure. The residue was diluted with H₂O and extracted with EtOAc. The combined organic phases were washed with brine, dried with Na₂SO₄ and the solvent was removed under reduced pressure to give 6-methoxy-5-[(tetrahydropyran-4-ylamino)methyl]pyrimidin-4-amine (3.1 g, 77% yield). LCMS (ESI): m/z: [M+H] calculated for C₁₁H₁₉N₄O₂: 239.1. found 239.1.

### Step 6.

To a solution of 6-methoxy-5-[(tetrahydropyran-4-ylamino)methyl]pyrimidin-4-amine (3.1 g, 13 mmol) in DCM (30 mL) was added triphosgene (39 g, 130 mmol) in DCM (15 mL) at 0° C. TEA (54 ml, 390 mmol) was added at 0° and the mixture was stirred at 0° C. for 30 min. The cooling bath was removed and the reaction mixture was allowed to stir at 25° C. for 2 h. Aqueous NaHCO₃ was added and the mixture was extracted with EtOAc. The combined organic layers were washed with brine, dried with Na₂SO₄ and the solvent was removed under reduced pressure. The crude residue was purified by column chromatography to give 5-methoxy-3-tetrahydropyran-4-yl-1,4-dihydropyrimido[4,5-d]pyrimidin-2-one (1.5 g, 44% yield). LCMS (ESI): m/z: [M+H] calculated for C₁₂H₁₆N₄O₃: 265.1. found 265.1.

### Step 7.

To a solution of 5-methoxy-3-tetrahydropyran-4-yl-1,4-dihydropyrimido[4,5-d]pyrimidin-2-one (400 mg, 1.5 mmol) in DMF (6 mL) was added K₂CO₃ (418 mg, 3.0 mmol) and CH₃I (188 μl, 3.0 mmol). The mixture was stirred at 25° C. for 2 h. The reaction mixture was diluted with H₂O and extracted with EtOAc. The combined organic layers were washed with brine, dried with Na₂SO₄ and the solvent was removed under reduced pressure. The crude residue was purified by prep-TLC to give 5-methoxy-1-methyl-3-tetrahydropyran-4-yl-4H-pyrimido[4,5-d]pyrimidin-2-one (135 mg, 32% yield). ¹H NMR (400 MHz, CHLOROFORM-d) δ=8.44 (s, 1H), 4.67-4.62 (m, 1H), 4.25 (s, 2H), 4.08-4.05 (m, 2H), 4.01 (s, 3H), 3.57-3.50 (m, 2H), 3.39 (s, 3H), 1.95-1.91 (m, 2H), 1.69-1.63 (m, 2H).

### Step 8.

A solution of 5-methoxy-1-methyl-3-tetrahydropyran-4-yl-4H-pyrimido[4,5-d]pyrimidin-2-one (135 mg, 485 μmol) in HBr (33% in AcOH, 2.5 ml, 15 mmol) was stirred at 100° C. for 1 h. After cooling to rt the solvent was removed under reduced pressure to give 5-hydroxy-1-methyl-3-tetrahydropyran-4-yl-4H-pyrimido[4,5-d]pyrimidin-2-one (130 mg, crude). LCMS (ESI): m/z: [M+H] calculated for C₁₂H₁₆N₄O₃: 265.1. found 265.1.

### Step 9.

To a solution of 5-hydroxy-1-methyl-3-tetrahydropyran-4-yl-4H-pyrimido[4,5-d] pyrimidin-2-one (130 mg, 492 μmol) and 3-[(1R)-1-aminoethyl]-5-(trifluoromethyl) aniline (110 mg, 541 μmol) in DMF (2 mL) was added BOP (326 mg, 738 μmol) and DBU (222 ml, 1.48 mmol). The mixture was stirred at 25° C. for 16 h. The reaction was diluted with H₂O and extracted with EtOAc. The combined organic phases were washed with brine, dried with Na₂SO₄ and the solvent was removed under reduced pressure. The residue was purified by prep-HPLC to give 5-[[(1R)-1-[3-amino-5-(trifluoromethyl)phenyl]ethyl]amino]-1-methyl-3-tetrahydropyran-4-yl-4H-pyrimido[4,5-d]pyrimidin-2-one (20 mg, 9% yield). LCMS (ESI): m/z: [M+H] calculated for C₂₁H₂₆F₃N₆O₂: 451.2. found 451.2; ¹H NMR (400 MHz, METHANOL-d₄) δ=8.14 (s, 1H), 6.94 (d, J=7.8 Hz, 2H), 6.84 (s, 1H), 5.42-5.30 (m, 1H), 4.60-4.48 (m, 1H), 4.28 (s, 2H), 4.08-4.03 (m, 2H), 3.61-3.47 (m, 2H), 3.28 (s, 3H), 2.12-2.00 (m, 2H), 1.64 (m, 2H), 1.56 (d, J=7.0 Hz, 3H).

### Example 348. Synthesis of (4-{1[(R)-1-[3-(difluoromethyl)-2-fluorophenyl]ethyl]amino}-6-(morpholine-4-carbonyl)-5H,6H,7H-pyrrolo[3,4-d]pyrimidin-2-yl)methanol

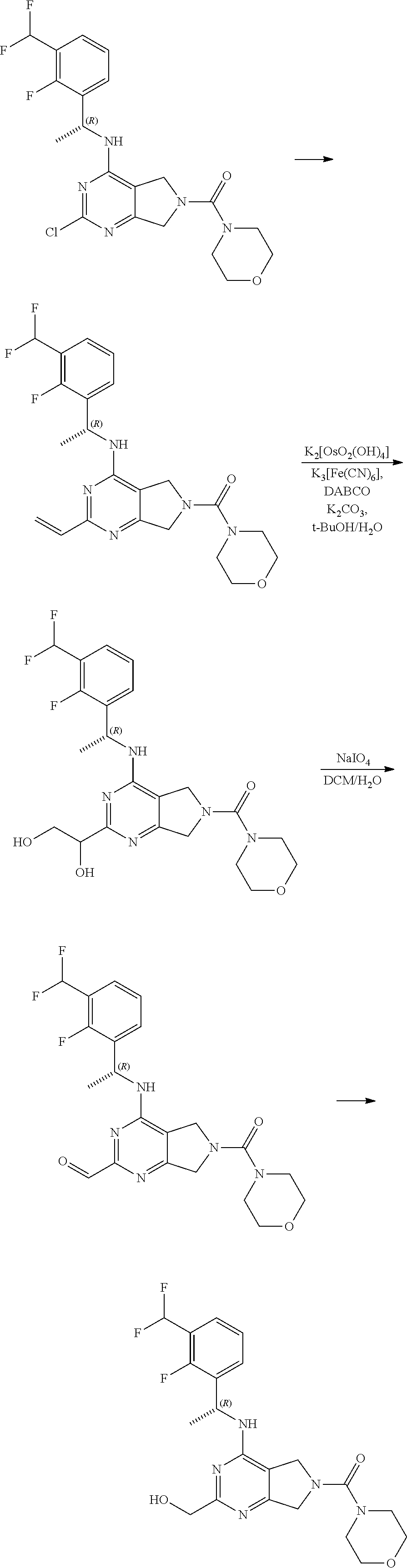

### Step 1.

N-[(1R)-1-[3-(difluoromethyl)-2-fluorophenyl]ethyl]-2-ethenyl-6-(morpholine-4-carbonyl)-5H,6H,7H-pyrrolo-[3,4-d]pyrimidin-4-amine was synthesized in a manner similar to 2-ethenyl-6-(morpholine-4-carbonyl)-N-[(1R)-1-[3-nitro-5-(trifluoromethyl)phenyl]ethyl]-5H,6H,7H-pyrrolo[3,4-d]pyrimidin-4-amine except 2-chloro-6-(morpholine-4-carbonyl)-N-[(1R)-1-[3-nitro-5-(trifluoromethyl)phenyl]ethyl]-5H,6H,7H-pyrrolo[3,4-d]pyrimidin-4-amine was substituted with 2-chloro-N-[(1R)-1-[3-(difluoromethyl)-2-fluorophenyl]ethyl]-6-(morpholine-4-carbonyl)-5H,6H,7H-pyrrolo[3,4-d]pyrimidin-4-amine to give (1.95 g, 100% yield). LCMS (ESI): m/z: [M+H] calculated for C₂₂H₂₄F₃N₅O₂: 447.19. found 448.40; ¹H NMR (300 MHz, DMSO-d₆) δ ppm 7.79 (d, J=7.1 Hz, 1H), 7.47 (t, J=7.0 Hz, 1H), 7.27 (t, J=7.7 Hz, 1H), 7.22 (t, J=54.3 Hz, 1H), 6.46 (dd, J=17.3, 10.3 Hz, 1H), 6.23 (dd, J=17.3, 2.5 Hz, 1H), 5.66-5.52 (m, 1H), 5.48 (dd, J=10.3, 2.5 Hz, 1H), 4.71-4.40 (m, 4H), 3.63 (t, J=4.7 Hz, 4H), 3.25 (t, J=4.7 Hz, 4H), 1.50 (d, J=7.0 Hz, 3H).

### Step 2.

To a mixture of N-[(1R)-1-[3-(difluoromethyl)-2-fluorophenyl]ethyl]-2-ethenyl-6-(morpholine-4-carbonyl)-5H,6H,7H-pyrrolo[3,4-d]pyrimidin-4-amine (1.93 g, 4.31 mmol) in t-BuOH (92.6 mL) and H₂O (92.6 mL) was added K3[Fe(CN)₆] (5.11 g, 15.5 mmol), K₂CO₃ (2.14 g, 15.5 mmol) and DABCO (34 mg, 0.30 mmol), followed by potassium osmate(VI) dihydrate (16 mg, 0.04 mmol). The mixture was stirred at rt for 12 h, then a solution of sodium bisulfite was added and the mixture extracted with EtOAc (×2). The combined organic layers were dried over anhydrous MgSO₄, filtered, concentrated under reduced pressure and the crude residue was purified by column chromatography to give 1-(4-{[(1R)-1-[3-(difluoromethyl)-2-fluorophenyl]ethyl]amino}-6-(morpholine-4-carbonyl)-5H,6H,7H-pyrrolo[3,4-d]pyrimidin-2-yl)ethane-1,2-diol (1.40 g, 68% yield). LCMS (ESI): m/z: [M+H] calculated for C₂₂H₂₆F₃N₅O₄: 481.19. found 482.10; H NMR (300 MHz, DMSO-d₆) δ ppm 7.82 (t, J=8.2 Hz, 1H), 7.64 (t, J=7.5 Hz, 1H), 7.49 (t, J=7.0 Hz, 1H), 7.29 (t, J=7.7 Hz, 1H), 7.22 (t, J=54.4 Hz, 1H), 5.61 (q, J=6.7 Hz, 1H), 4.68 (d, J=5.9 Hz, 1H), 4.66-4.49 (m, 4H), 4.44 (t, J=6.0 Hz, 1H), 4.39-4.12 (m, 1H), 3.64 (t, J=5.6, 3.7 Hz, 4H), 3.58-3.39 (m, 2H), 3.25 (t, J=4.7 Hz, 4H), 1.51 (d, J=7.0 Hz, 3H).

### Step 3.

To a mixture of 1-(4-{[(1R)-1-[3-(difluoromethyl)-2-fluorophenyl]ethyl]amino}-6-(morpholine-4-carbonyl)-5H,6H,7H-pyrrolo[3,4-d]pyrimidin-2-yl)ethane-1,2-diol (1.40 g, 2.9 mmol) in DCM (89.7 mL) and H₂O (11.2 mL) was added silica gel (22.4 g) and NaIO₄ (1.87 g, 8.7 mmol). The mixture was stirred at rt for 12 h, then filtered through a pad of Celite® and the filter cake washed with DCM. The filtrate was diluted with H₂O and the layers partitioned, then the aqueous layer was extracted with DCM. The combined organic layers were dried over MgSO₄, filtered and concentrated under reduced pressure to give 4-{[(1R)-1-[3-(difluoromethyl)-2-fluorophenyl]ethyl]amino}-6-(morpholine-4-carbonyl)-5H,6H,7H-pyrrolo[3,4-d]pyrimidine-2-carbaldehyde (1.26 g, 96% yield). LCMS (ESI): m/z: [M+H] calculated for C₂₁H₂₂F₃N₅O₃: 449.17. found 450.05; ¹H NMR (300 MHz, DMSO-d₆) δ ppm 9.66 (s, 1H), 8.14 (d, J=7.5 Hz, 1H), 7.67 (t, J=7.4 Hz, 1H), 7.51 (t, J=7.1 Hz, 1H), 7.30 (t, J=7.7 Hz, 1H), 7.24 (t, J=54.4 Hz, 1H), 5.75-5.64 (m, 1H), 4.86-4.29 (m, 4H), 3.65 (t, J=5.6, 3.7 Hz, 4H), 3.27 (t, J=4.7 Hz, 4H), 1.55 (d, J=7.0 Hz, 3H).

### Step 4.

(4-{[(1R)-1-[3-(difluoromethyl)-2-fluorophenyl]ethyl]amino}-6-(morpholine-4-carbonyl)-5H,6H,7H-pyrrolo[3,4-d]pyrimidin-2-yl)methanol was synthesized in a manner similar to [6-(morpholine-4-carbonyl)-4-{[(1R)-1-[3-nitro-5-(trifluoromethyl)phenyl]ethyl]amino}-5H,6H,7H-pyrrolo[3,4-d]pyrimidin-2-yl]-methanol except 6-(morpholine-4-carbonyl)-4-{[(1R)-1-[3-nitro-5-(trifluoromethyl)phenyl]-ethyl]amino}-5H,6H,7H-pyrrolo[3,4-d]pyrimidine-2-carbaldehyde was substituted with 4-{[(JR)-1-[3-(difluoromethyl)-2-fluorophenyl]ethyl]amino}-6-(morpholine-4-carbonyl)-5H,6H,7H-pyrrolo[3,4-d]pyrimidine-2-carbaldehyde to give (120 mg, 70% yield). ¹H NMR (300 MHz, DMSO-d₆) δ ppm 7.78 (d, J=7.6 Hz, 1H), 7.64 (t, J=7.4 Hz, 1H), 7.50 (t, J=7.1 Hz, 1H), 7.29 (t, J=7.7 Hz, 1H), 7.22 (t, J=54.4 Hz, 1H), 5.66 (t, J=7.2 Hz, 1H), 4.67 (t, J=5.9 Hz, 1H), 4.63-4.46 (m, 4H), 4.34-4.18 (m, 2H), 3.64 (t, J=4.6 Hz, 4H), 3.26 (t, J=4.7 Hz, 4H), 1.50 (d, J=7.0 Hz, 3H).

### Example 445. (R)-5-((1-(3-(difluoromethyl)-2-fluorophenyl)ethyl)amino)-1,7-dimethyl-3-(1-methylpiperidin-4-yl)-3,4-dihydropyrimido[4,5-d]pyrimidin-2(1H)-one

(R)-5-((1-(3-(difluoromethyl)-2-fluorophenyl)ethyl)amino)-1,7-dimethyl-3-(1-methylpiperidin-4-yl)-3,4-dihydropyrimido[4,5-d]pyrimidin-2(1H)-one was synthesized in the manner similar to Example 176.

| | | Mass |
|---|---|---|
| Example # | Structure | Found |
| Example 445. | 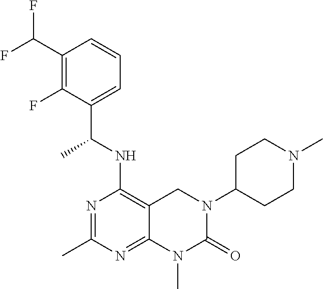 [SMILES: Cc1nc(N[C@H](C)c2cccc(C(F)F)c2F)c2c(n1)N(C)C(=O)N(C1CCN(C)CC1)C2] | 463.3 |

### Example 446. Synthesis of [2-(azetidin-3-yl)-4-[[(1R)-1-[3-(difluoromethyl)-2-fluoro-phenyl]ethyl]amino]-5,7-dihydropyrrolo[3,4-d]pyrimidin-6-yl]-morpholino-methanone

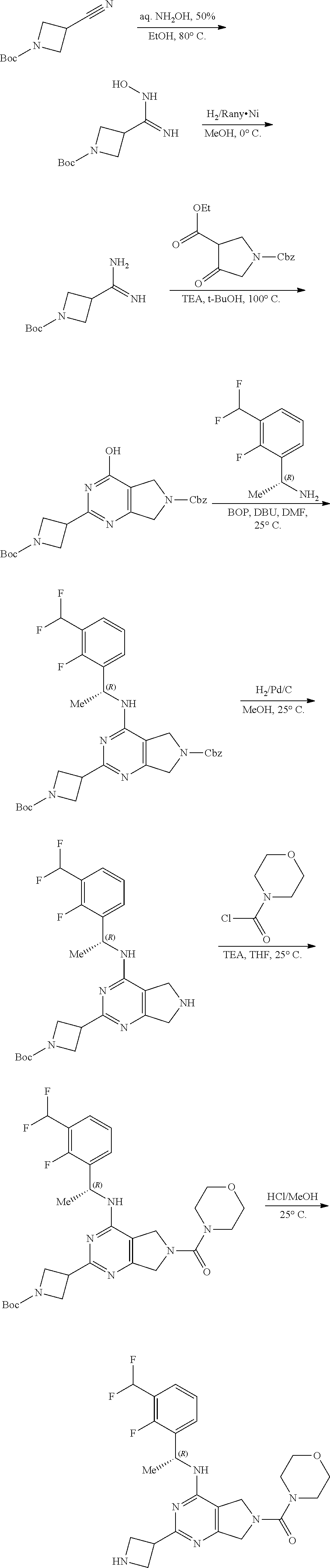

### Step 1.

To a solution of tert-butyl 3-cyanoazetidine-1-carboxylate (1 g, 5.49 mmol) in EtOH (25 mL) was added aq. hydroxylamine (181.26 mg, 5.49 mmol, 1 mL) under N₂. The mixture was heated to 80° C. and stirred for 24 hours. The reaction mixture was concentrated under reduced pressure to give tert-butyl 3-(N-hydroxycarbamimidoyl)azetidine-1-carboxylate (1.3 g, crude). LCMS (ESI): m/z: [M+H] calculated for C₉H₁₈N₃O₃: 216.1. found 216.2; ¹H NMR (400 MHz, DMSO-d₆) δ=9.10 (s, 1H), 5.48 (s, 2H), 3.89 (s, 4H), 3.09-3.23 (m, 1H), 1.38 (s, 9H).

### Step 2.

To a solution of tert-butyl 3-(N-hydroxycarbamimidoyl)azetidine-1-carboxylate (1 g, 4.65 mmol) in MeOH (25 mL) was added Raney-Ni (200 mg, 2.33 mmol) under N₂. The suspension was degassed under vacuum and purged with H₂ several times. The mixture was stirred under H₂ at 0° C. for 8 hours. The reaction mixture was filtered, and the filtrate was concentrated under reduced pressure to give tert-butyl 3-carbamimidoylazetidine-1-carboxylate (900 mg, crude). LCMS (ESI): m/z: [M+H] calculated for C₉H₁₈N₃O₂: 200.1. found 200.2; ¹H NMR (400 MHz, DMSO-d₆) δ=5.99 (s, 2H), 3.96-3.77 (m, 4H), 3.25-3.07 (m, 1H), 1.41-1.31 (m, 9H).

### Step 3.

To a solution of tert-butyl 3-carbamimidoylazetidine-1-carboxylate (800 mg, 4.02 mmol) and O1-benzyl O3-ethyl 4-oxopyrrolidine-1,3-dicarboxylate (1.29 g, 4.42 mmol) in t-BuOH (16 mL) was added TEA (1.22 g, 12.05 mmol, 1.68 mL) in one portion at rt under N₂. The mixture was heated to 100° C. and stirred for 4 hours. The reaction was quenched with H₂O (20 mL) slowly and then extracted with EtOAc (50 mL×3). The combined organic phase was washed with brine (50 mL), dried over anhydrous Na₂SO₄, filtered and concentrated in vacuo. The residue was purified by column chromatography to give benzyl 2-(1-tert-butoxycarbonylazetidin-3-yl)-4-hydroxy-5,7-dihydropyrrolo[3,4-d]pyrimidine-6-carboxylate (1.3 g, 75.92% yield). LCMS (ESI): m/z: [M+H] calculated for C₂₂H₂₇N₄O₅: 427.2. found 427.3.

### Step 4.

To a mixture of benzyl 2-(1-tert-butoxycarbonylazetidin-3-yl)-4-hydroxy-5,7-dihydropyrrolo [3,4-d]pyrimidine-6-carboxylate (800 mg, 1.88 mmol) and (1R)-1-[3-(difluoromethyl)-2-fluoro-phenyl]ethanamine (425.85 mg, 2.25 mmol) in DMF (16 mL) was added BOP (1.33 g, 3.00 mmol), DBU (856.74 mg, 5.63 mmol, 848.26 μL) in one portion at 15° C. under N₂. The mixture was stirred at 15° C. for 12 hours. The reaction was quenched with H₂O (20 mL) slowly and then extracted with EtOAc (20 mL×3). The combined organic phase was washed with brine (20 mL), dried over anhydrous Na₂SO₄, filtered and concentrated in vacuo. The residue was purified by column chromatography to give benzyl 2-(1-tert-butoxycarbonylazetidin-3-yl)-4-[[(1R)-1-[3-(difluoromethyl)-2-fluoro-phenyl]ethyl]amino]-5,7-dihydropyrrolo[3,4-d]pyrimidine-6-carboxylate (300 mg, 26.76% yield). LCMS (ESI): m/z: [M+H] calculated for C₃₁H₃₅F₃N₅O₄: 598.3. found 598.4.

### Step 5.

To a solution of benzyl 2-(1-tert-butoxycarbonylazetidin-3-yl)-4-[[(1R)-1-[3-(difluoromethyl)-2-fluoro-phenyl]ethyl]amino]-5,7-dihydropyrrolo[3,4-d]pyrimidine-6-carboxylate (100 mg, 167.33 umol) in MeOH (2 mL) was added Pd/C (10 mg) under N₂. The suspension was degassed under vacuum and purged with H₂ several times. The mixture was stirred under H₂ at rt for 3 hours. The reaction mixture was filtered and the filtrate was concentrated under reduced pressure to give tert-butyl-3-[4-[[(1R)-1-[3-(difluoromethyl)-2-fluoro-phenyl]ethyl]amino]-6,7-dihydro-5H-pyrrolo[3,4-d]pyrimidin-2-yl]azetidine-1-carboxylate (70 mg, 90.26% yield). LCMS (ESI): m/z: [M+H] calculated for C₂₃H₂₉F₃N₅O₂: 464.2. found 464.4.

### Step 6.

To a solution of tert-butyl 3-[4-[[(1R)-1-[3-(difluoromethyl)-2-fluoro-phenyl]ethyl]amino]-6,7-dihydro-5H-pyrrolo[3,4-d]pyrimidin-2-yl]azetidine-1-carboxylate (70 mg, 151.03 umol) and morpholine-4-carbonyl chloride (22.59 mg, 151.03 umol, 17.65 μL) in THF (1 mL) was added TEA (61.13 mg, 604.11 umol, 84.08 μL) in one portion at rt under N₂. The mixture was stirred at rt for 1 hour. The reaction mixture was concentrated under reduced pressure and the residue was purified by column chromatography to give tert-butyl 3-[4-[[(1R)-1-[3-(difluoromethyl)-2-fluoro-phenyl]ethyl]amino]-6-(morpholine-4-carbonyl)-5,7-dihydropyrrolo[3,4-d]pyrimidin-2-yl]azetidine-1-carboxylate (43 mg, 49.388% yield). LCMS (ESI): m/z: [M+H] calculated for C₂₈H₃₆F₃N₆O₄: 577.3. found 577.2.

### Step 7.

To a mixture of tert-butyl 3-[4-[[(1R)-1-[3-(difluoromethyl)-2-fluoro-phenyl]ethyl]amino]-6-(morpholine-4-carbonyl)-5,7-dihydropyrrolo[3,4-d]pyrimidin-2-yl]azetidine-1-carboxylate (43 mg, 74.57 umol) in MeOH (2 mL) was added HCl/MeOH (4 M, 1 mL) in one portion at rt under N₂. The mixture was stirred at rt for 5 hours. The reaction mixture was concentrated under reduced pressure. The residue was purified by prep-HPLC to give [2-(azetidin-3-yl)-4-[[(1R)-1-[3-(difluoromethyl)-2-fluoro-phenyl]ethyl]amino]-5,7-dihydropyrrolo[3,4-d]pyrimidin-6-yl] morpholino-methanone (12 mg, 33.56% yield). LCMS (ESI): m/z: [M+H] calculated for C₂₃H₂₈F₃N₆O₂: 477.2. found; 477.2; ¹H NMR (400 MHz, METHANOL-d₄) δ=7.55-7.62 (m, 1H), 7.44-7.51 (m, 1H), 7.22-7.28 (m, 1H), 6.85-7.16 (m, 1H), 5.67-5.75 (m, 1H), 4.66-4.72 (m, 2H), 4.58-4.64 (m, 2H), 4.18-4.37 (m, 3H), 4.09-4.17 (m, 1H), 3.98-4.09 (m, 1H), 3.71-3.77 (m, 4H), 3.35-3.40 (m, 4H), 1.59-1.63 (m, 3H).

### Example 447. (R)-(4-((1-(3-(difluoromethyl)-2-fluorophenyl)ethyl)amino)-2-(1-methylazetidin-3-yl)-5,7-dihydro-6H-pyrrolo[3,4-d]pyrimidin-6-yl)(morpholino)methanone

(R)-(4-((1-(3-(difluoromethyl)-2-fluorophenyl)ethyl)amino)-2-(1-methylazetidin-3-yl)-5,7-dihydro-6H-pyrrolo[3,4-d]pyrimidin-6yl)(morpholino)methanone was synthesized in the manner similar to Example 446.

| | | Mass |
|---|---|---|
| Example # | Structure | Found |
| Example 447. | 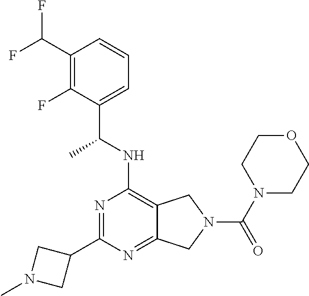 [SMILES: C[C@@H](Nc1nc(C2CN(C)C2)nc2c1CN(C(=O)N1CCOCC1)C2)c1cccc(C(F)F)c1F] | 491.2 |

### Example 448. Synthesis of (R)—N-(1-(3-amino-5-(trifluoromethyl)phenyl)ethyl)-2-chloro-6-((1-(methoxymethyl)cyclobutyl)sulfonyl)-5,6,7,8-tetrahydropyrido[4,3-d]pyrimidin-4-amine

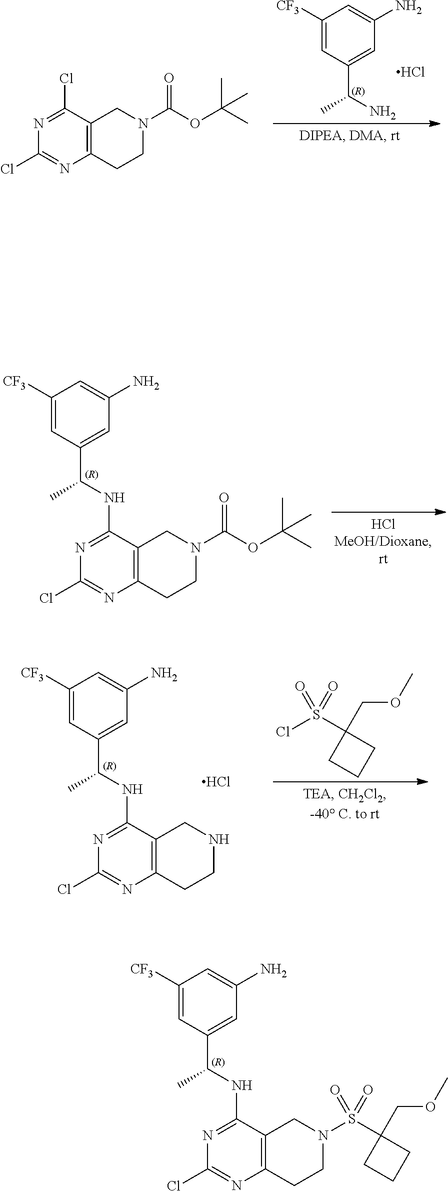

### Step 1

To a mixture of 3-[(1R)-1-aminoethyl]-5-(trifluoromethyl)aniline (4.02 g, 19.6 mmol) and tert-butyl 2,4-dichloro-5H,6H,7H,8H-pyrido[4,3-d]pyrimidine-6-carboxylate (6.00 g, 19.7 mmol) in DMA (40 mL), DIPEA (30.0 mL, 172 mmol) was added. The reaction mixture was stirred overnight at rt. The reaction was quenched with water, extracted with EtOAc, treated with brine, dried over MgSO₄, filtered, and concentrated under reduced pressure to a red oil. This material was purified by flash chromatography to give tert-butyl 4-{[(1R)-1-[3-amino-5-(trifluoromethyl)phenyl]ethyl]amino}-2-chloro-5H,6H,7H,8H-pyrido[4,3-d]pyrimidine-6-carboxylate (6.92 g, 74% yield). LCMS (ESI): m/z: [M+H] calculated for C₂₁H₂₆ClF₃N₅O₂: 472.2. found: 472.3.

### Step 2

To a solution of tert-butyl 4-{[(1R)-1-[3-amino-5-(trifluoromethyl)phenyl]ethyl]amino}-2-chloro-5H,6H,7H,8H-pyrido[4,3-d]pyrimidine-6-carboxylate (6.92 g, 14.6 mmol) in methanol (10 mL), a 4 M solution of HCl in dioxane (30 mL, 120 mmol) was added. The reaction was stirred at rt for 1 hour, then concentrated under vacuum to give N-[(1R)-1-[3-amino-5-(trifluoromethyl)phenyl]ethyl]-2,6-dichloro-5H,6H,7H,8H-pyrido[4,3-d]pyrimidin-4-amine (5.32 g, 89% yield). LCMS (ESI): m/z: [M+H] calculated for C₁₋₆H₁₈ClF₃N₅: 372.1. found: 372.4.

### Step 3

To a solution of N-[(1R)-1-[3-amino-5-(trifluoromethyl)phenyl]ethyl]-2,6-dichloro-5H,6H,7H,8H-pyrido[4.3-d]pyrimidin-4-amine (106 mg, 260 μmol) and triethylamine (358 μL, 2.58 mmol) in DCM (5.0 mL) at −40° C., 1-(methoxymethyl)cyclobutane-1-sulfonyl chloride (159 mg, 578 μmol) was added. The cooling bath was removed and the reaction was stirred at rt for 2 days. The reaction was then concentrated under reduced pressure and the resulting solid was purified by prep-HPLC to give N-[(1R)-1-[3-amino-5-(trifluoromethyl)phenyl]ethyl]-2-chloro-6-{[1-(methoxymethyl)cyclobutyl]sulfonyl}-5H,6H,7H,8H-pyrido[4,3-d]pyrimidin-4-amine (37.7 mg, 27% yield). LCMS (ESI): m/z: [M+H] calculated for C₂₂H₂₈ClF₃N₅O₃S: 534.15. found: 534.5; ¹H NMR (500 MHz, Methanol-d₄) δ 6.83 (s, 1H), 6.80 (s, 1H), 6.70 (s, 1H), 5.28 (q, J=7.0 Hz, 1H), 4.19-4.10 (m, 2H), 3.65 (s, 2H), 3.55-3.47 (m, 2H), 3.15 (s, 3H), 2.65-2.56 (m, 4H), 2.09-1.91 (m, 4H), 1.45 (d, J=7.0 Hz, 3H).

The following examples 449-459 shown in Table 7 were synthesized in the manner similar to Example xx.

TABLE 7
Examples 449-459

| Example # | Structure | Mass Found |
|---|---|---|
| Example 449. | 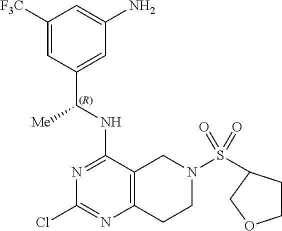 | 506.5 |
| Example 450. | 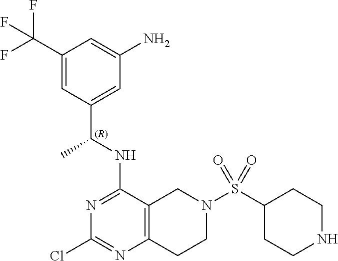 | 519.5 |
| Example 451. | 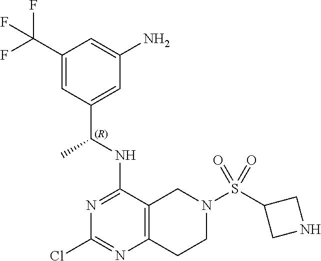 | 491.5 |
| Example 452. | 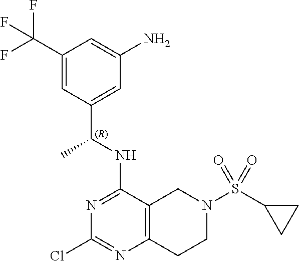 | 476.4 |
| Example 453. | 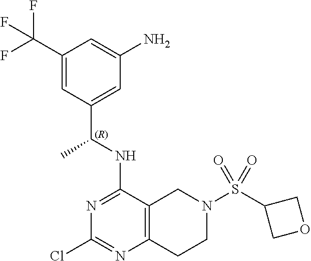 | 492.5 |
| Example 454. | 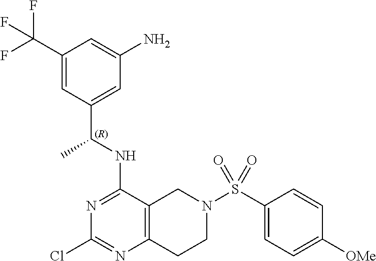 | 542.5 |
| Example 455. | 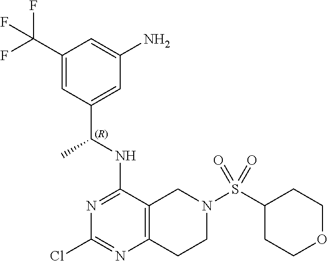 | 520.5 |
| Example 456. | 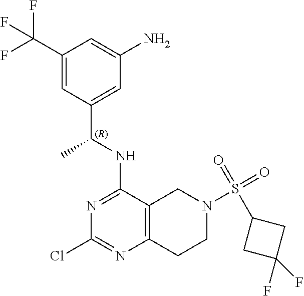 | 526.5 |
| Example 457. | 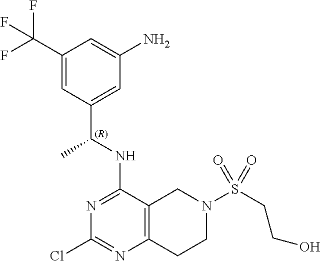 | 480.5 |
| Example 458. | 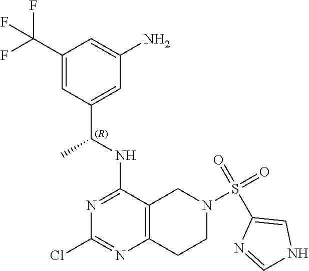 | 502.4 |
| Example 459. | 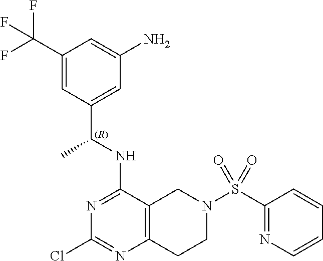 | 513.5 |

### Example 538. and 460. Synthesis of (2-chloro-4-(((1R)-1-(3-(difluoro(morpholin-3-yl)methyl)phenyl)ethyl)amino)-5,7-dihydro-6H-pyrrolo[3,4-d]pyrimidin-6-yl)(morpholino)methanone (Example 538.) and (2-chloro-4-(((1R)-1-(3-(difluoro(4-methylmorpholin-3-yl)methyl)phenyl)ethyl)amino)-5,7-dihydro-6H-pyrrolo[3,4-d]pyrimidin-6-yl)(morpholino)methanone (Example 460.)

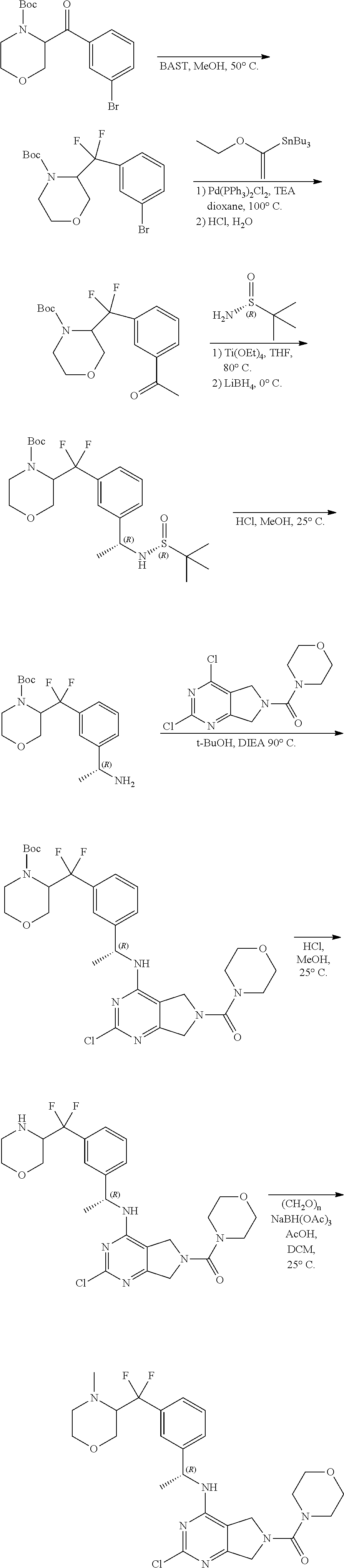

### Step 1.

To a mixture of tert-butyl 3-(3-bromobenzoyl)morpholine-4-carboxylate (1.1 g, 2.97 mmol) in BAST (10 mL) was added MeOH (12.0 μL, 297 μmol) at 25° C. The mixture was stirred at 50° C. for 6 h, then quenched with H₂O (5 mL), extracted with EtOAc, dried over Na₂SO₄, filtered, and concentrated under reduced pressure. The residue was purified by column chromatography to give tert-butyl 3-[(3-bromophenyl)-difluoro-methyl]morpholine-4-carboxylate (500 mg, 43% yield). ¹H NMR (400 MHz, METHANOL-d₄) δ ppm 7.70-7.58 (m, 2H), 7.48-7.35 (m, 2H), 4.42-4.23 (m, 2H), 3.95-3.83 (m, 2H), 3.69-3.58 (m, 1H), 3.47-3.40 (m, 1H), 1.31-1.22 (m, 9H).

### Step 2.

A mixture of tert-butyl 3-[(3-bromophenyl)-difluoro-methyl]morpholine-4-carboxylate (500 mg, 1.27 mmol), tributyl(1-ethoxyvinyl)stannane (691 mg, 1.91 mmol), TEA (444 μL, 3.19 mmol), and Pd(PPh₃)₂Cl₂ (89.5 mg, 127 μmol) in dioxane (5 mL) was sparged with N₂ then the mixture was stirred at 100° C. for 4 h under an atmosphere of N₂. The reaction was quenched with a 2M aqueous solution of HCl (5 mL) and the mixture was stirred for 1 h then filtered. The filtrate was extracted with EtOAc (30 mL×2) and the combined organic extracts were treated with water (20 mL) and KF (100 mg) and the mixture was stirred for 2 h. The mixture was filtered and the organic layer was treated with brine (5 mL), dried over Na₂SO₃, filtered, and concentrated under vacuum. The crude residue was purified by prep-TLC to give tert-butyl 3-[(3-acetylphenyl)-difluoro-methyl]morpholine-4-carboxylate (300 mg, 66% yield). ¹H NMR (400 MHz, METHANOL-d₄) δ ppm 8.17-8.08 (m, 2H), 7.80-7.58 (m, 2H), 4.48-4.24 (m, 2H), 3.92 (d, 2H), 3.65 (dd, J=12.8, 4.0 Hz, 1H), 3.53-3.38 (m, 2H), 2.66 (s, 3H), 1.29-1.16 (m, 9H).

### Step 3.

To a solution of tert-butyl 3-[(3-acetylphenyl)-difluoro-methyl]morpholine-4-carboxylate (270 mg, 760 μmol) in THF (2 mL) were added Ti(OEt)₄ (473 μL, 2.28 mmol) and 2-methylpropane-2-sulfinamide (184 mg, 1.52 mmol). The mixture was stirred at 80° C. for 7 h. After cooling to −4° C., MeOH (30.7 μL, 760 μmol) was added, followed by then LiBH₄ (49.6 mg, 2.28 mmol) and the mixture was stirred at 0° C. for 1 h. The reaction was poured slowly into H₂O (4 mL) and THF (4 mL), filtered over Celite®, washed with THF, and evaporated under reduced pressure. The crude residue was purified by prep-TLC to give t-butyl 3-[[3-[(1R)-1-[[(R)-tert-butylsulfinyl]amino]ethyl]phenyl]-difluoro-methyl]morpholine-4-carboxylate (220 mg, 63% yield). ¹H NMR (400 MHz, METHANOL-d₄) δ ppm 7.58-7.37 (m, 4H), 4.58-4.50 (m, 1H), 4.22-4.14 (m, 1H), 3.95-3.86 (m, 1H), 3.58 (dd, J=12.4, 3.6 Hz, 1H), 3.51-3.35 (m, 2H), 1.53 (d, J=6.8 Hz, 3H), 1.39-1.25 (m, 9H), 1.24 (s, 9H)

### Step 4.

To a solution of tert-butyl 3-[[3-[(1R)-1-[[(R)-tert-butylsulfinyl]amino]ethyl]phenyl]-difluoro-methyl]morpholine-4-carboxylate (270 mg, 586 μmol) in MeOH (2 mL), a 4 M solution of HCl in MeOH (293 μL, 1.17 mmol) was added. The reaction was stirred at 25° C. for 30 min, then was quenched by the addition of NaOH until pH=7. The resulting mixture was filtered and concentrated under reduced pressure to give tert-butyl 3-[[3-[(1R)-1-aminoethyl]phenyl]-difluoro-methyl]morpholine-4-carboxylate (200 mg, 96% yield). ¹H NMR (400 MHz, METHANOL-d₄) δ ppm 7.61-7.34 (m, 4H), 4.26-4.16 (m, 2H), 4.01-3.85 (m, 2H), 3.64-3.60 (m, 1H), 3.57-3.37 (m, 3H), 1.52-1.41 (m, 3H), 1.38-1.16 (m, 9H).

### Step 5.

To a mixture of tert-butyl 3-[[3-[(1R)-1-aminoethyl]phenyl]-difluoro-methyl]morpholine-4-carboxylate (100 mg, 280 μmol) in t-BuOH (2 mL), DIPEA (97.7 μL, 561 μmol) and (2,4-dichloro-5,7-dihydropyrrolo[3,4-d]pyrimidin-6-yl)-morpholino-methanone (128 mg, 421 μmol) were added. The mixture was stirred at 90° C. for 1 h, then was quenched by addition of H₂O, extracted with EtOAc, dried over Na₂SO₃, filtered, and concentrated under reduced pressure. The crude residue was purified by prep-TLC to give tert-butyl 3-[[3-[(1R)-1-[[2-chloro-6-(morpholine-4-carbonyl)-5,7-dihydropyrrolo[3,4-d]pyrimidin-4-yl]amino]ethyl]phenyl]-difluoro-methyl]morpholine-4-carboxylate (80 mg, 46% yield). LCMS (ESI): m/z: [M+H] calculated for C₂₉H₃₈ClF₂N₆O₅: 623.2. found 623.2.

### Step 6.

A solution of 3-[[3-[(1R)-1-[[2-chloro-6-(morpholine-4-carbonyl)-5,7-dihy dropyrrolo[3,4-d]pyrimidin-4-yl]amino]ethyl]phenyl]-difluoro-methyl]morpholine-4-carboxylate (80 mg, 128.39 μmol) in a 4M HCl solution in methanol (32 μL, 128 μmol) was stirred at 25° C. for 1 h. The mixture was concentrated under reduced pressure and the crude residue was purified by prep-HPLC to give [2-chloro-4-[[(1R)-1-[3-[difluoro(morpholin-3-yl)methyl]phenyl]ethyl]amino]-5,7-dihydropyrrolo[3,4-d]pyrimidin-6-yl]-morpholino-methanone (27.0 mg, 40% yield). LCMS (ESI): m/z: [M+H] calculated for C₂₄H₃₀ClF₂N₆O₃: 523.2. found 523.2; ¹H NMR (400 MHz, METHANOL-d₄) δ ppm 7.60-7.53 (m, 2H), 7.47 (t, J=7.2 Hz, 1H), 7.39 (d, J=7.2 Hz, 1H), 5.39 (q, J=6.4 Hz, 1H), 4.62-4.56 (m, 4H), 3.80-3.67 (m, 6H), 3.53-3.41 (m, 3H), 3.38-3.34 (m, 4H), 2.94-2.88 (m, 2H), 1.60 (d, J=7.2 Hz, 3H).

### Step 7.

To a solution of [2-chloro-4-[[(1R)-1-[3-[difluoro(morpholin-3-yl)methyl]phenyl]ethyl]amino]-5,7-dihydropyrrolo[3,4-d]pyrimidin-6-yl]-morpholino-methanone (50 mg, 96 μmol) in AcOH (1.2 mL) and DCM (3 mL), NaBH(OAc)₃ (50.7 mg, 239 μmol) and paraformaldehyde (50 mg) were added. The reaction was stirred at 25° C. for 12 h, then was concentrated under reduced pressured and purified by prep-HPLC to give [2-chloro-4-[[(1R)-1-[3-[difluoro-(4-methylmorpholin-3-yl)methyl]phenyl]ethyl]amino]-5,7-dihydropyrrolo[3,4-d]pyrimidin-6-yl]-morpholino-methanone (30 mg, 58% yield). LCMS (ESI): m/z: [M+H] calculated for C₂₅H₃₂ClF₂N₆O₃: 537.2. found 537.2; ¹H NMR (400 MHz, METHANOL-d₄) δ ppm 7.55-7.50 (m, 2H), 7.44-7.33 (m, 2H), 5.40-5.31 (m, 1H), 4.63-4.54 (m, 4H), 3.74-3.65 (m, 5H), 3.51-3.41 (m, 2H), 3.45-3.35 (m, 4H), 3.25-3.16 (m, 2H), 2.93-2.84 (m, 1H), 2.81-2.74 (m, 1H), 2.53-2.45 (m, 3H), 2.43-2.37 (m, 1H), 1.59 (d, J=7.2 Hz, 3H).

### Example 461. Synthesis of (4-((1-(5-amino-2-fluoro-3-(trifluoromethyl)phenyl)ethyl)amino)-2-chloro-5,7-dihydro-6H-pyrrolo[3,4-d]pyrimidin-6-yl)(morpholino)methanone

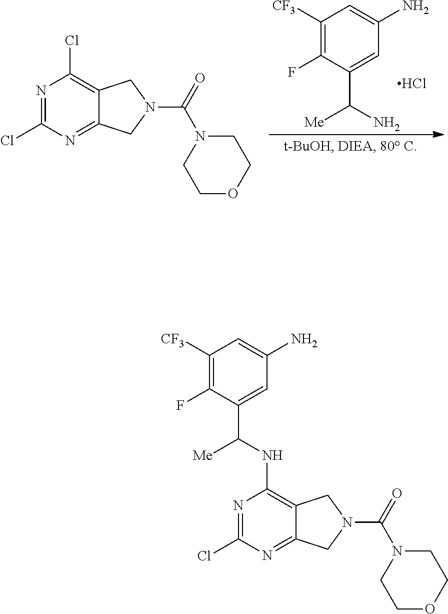 [SMILES: O=C(N1CCOCC1)N1Cc2nc(Cl)nc(Cl)c2C1 ; CC(N)c1cc(N)cc(C(F)(F)F)c1F ; Cl ; CC(Nc1nc(Cl)nc2c1CN(C(=O)N1CCOCC1)C2)c1cc(N)cc(C(F)(F)F)c1F]

To a solution of 3-(1-aminoethyl)-4-fluoro-5-(trifluoromethyl)aniline HCl salt (5.0 mg, 19 μmol) in t-BuOH (0.5 mL), DIEA (10.1 μL, 58.0 μmol) and (2,4-dichloro-5,7-dihydropyrrolo[3,4-d]pyrimidin-6-yl)-morpholino-methanone (5.86 mg, 19.3 μmol) were added. The mixture was stirred at 80° C. for 2 h, then was concentrated under reduced pressure. The crude residue was purified by prep-HPLC to give [4-[1-[5-amino-2-fluoro-3-(trifluoromethyl)phenyl]ethylamino]2-chloro-5,7-dihydropyrrolo[3,4-d]pyrimidin-6-yl]-morpholino-methanone (3.0 mg, 32% yield). LCMS (ESI): m/z: [M+H] calculated for C₂₀H₂₂ClF₄N₆O₂: 489.1. found 489.1; ¹H NMR (400 MHz, METHANOL-d₄) δ ppm 6.90-6.87 (m, 1H), 6.83-6.79 (m, 1H), 5.62-5.45 (m, 1H) 4.73-4.54 (m, 4H), 3.76-3.71 (m, 4H), 3.39-3.30 (m, 4H), 1.55 (d, J=7.0 Hz, 3H).

### Example 462. Synthesis of 2-chloro-4-((1-(3-(pentafluoro-16-sulfaneyl)phenyl)ethyl)amino)-5,7-dihydro-6H-pyrrolo[3,4-d]pyrimidin-6-yl)(morpholino)methanone

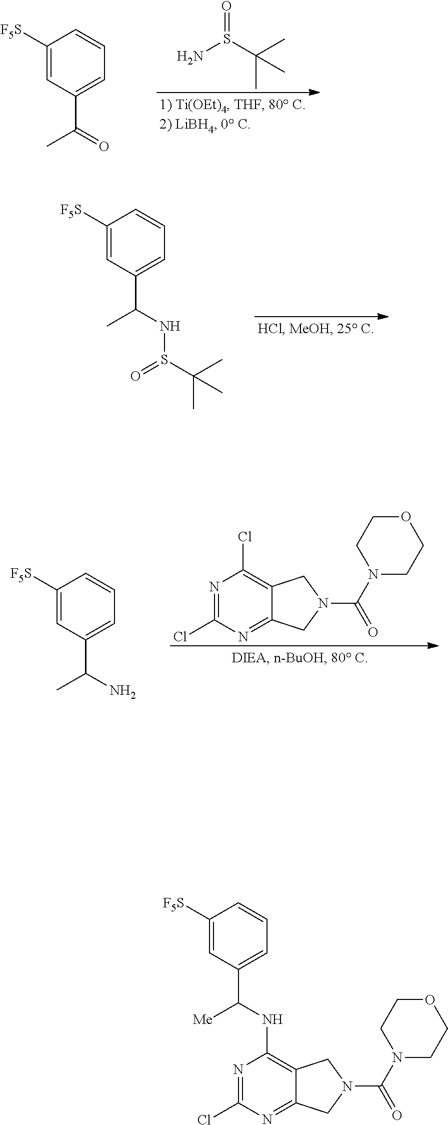

### Step 1.

To mixture of 1-[3-(pentafluoro-sulfanyl)phenyl]ethanone (100 mg, 406 μmol) and 2-methylpropane-2-sulfinamide (98.5 mg, 812 μmol) in THF (1.5 mL) was added Ti(OEt)₄ (253 μL, 1.22 mmol) at 25° C. The reaction was heated to 90° C. and stirred for 3 h. The mixture was then cooled to 0° C., then LiBH₄ (8.85 mg, 406 μmol) and MeOH (16.4 μL, 406 μmol) were added and the mixture was stirred at 0° C. for 30 min. The mixture was quenched by the addition of H₂O at 0° C. and filtered. The solvent was removed under reduced pressure and the residue was purified by column chromatography to give 2-methyl-N-[1-[3-(pentafluoro-sulfanyl)phenyl]ethyl]propane-2-sulfinamide (60 mg, 42% yield). ¹H NMR (400 MHz, CHLOROFORM-d) δ ppm 7.77-7.66 (m, 2H), 7.55-7.51 (m, 1H), 7.50-7.44 (m, 1H), 4.67-4.58 (m, 1H), 3.49-3.45 (m, 1H), 1.56 (d, J=6.6 Hz, 3H), 1.25 (s, 9H).

### Step 2.

To a solution of 2-methyl-N-[1-[3-(pentafluoro-sulfanyl)phenyl]ethyl]propane-2-sulfinamide (60 mg, 170 μmol, 1 eq) in MeOH (0.5 mL) was added 4M HCl in MeOH (128 μL, 512 μmol). The mixture was stirred at 25° C. for 30 min. The solvent was removed under reduced pressure to give 1-[3-(pentafluoro-sulfanyl)phenyl]ethanamine (60 mg). ¹H NMR (400 MHz, CHLOROFORM-d) δ ppm 7.93 (s, 1H), 7.77 (d, J=8.1 Hz, 1H), 7.69 (d, J=7.1 Hz, 1H), 7.54-7.48 (m, 1H), 3.82-3.81 (m, 1H), 1.75-1.66 (m, 3H).

### Step 3.

To a solution of 1-[3-(pentafluoro-sulfanyl)phenyl]ethanamine (60 mg, 240 μmol) in n-BuOH (2 mL) was added (2,4-dichloro-5,7-dihydropyrrolo[3,4-d]pyramidin-6-yl)-morpholino-methanone (73.6 mg, 243 μmol) and DIEA (211 μL, 1.21 mmol). The mixture was stirred at 80° C. for 12 h and then concentrated under reduced pressure. The crude residue was purified by prep-HPLC to give [2-chloro-4-[1-[3-(pentafluoro-sulfanyl)phenyl]ethylamino]-5,7-dihydropyrrolo[3,4-d]pyrimidin-6-yl]-morpholino-methanone (26 mg, 20% yield). LCMS (ESI): m/z: [M+H] calculated for C₁₉H₂₂ClF₅N₅O₂S: 514.1. found 514.1; ¹H NMR (400 MHz, CHLOROFORM-d) δ ppm 7.80-7.78 (m, 1H), 7.72-7.67 (m, 1H), 7.57-7.53 (m, 1H), 7.51-7.44 (m, 1H), 5.50-5.43 (m, 1H), 4.91 (d, J=6.2 Hz, 1H), 4.66-4.54 (m, 4H), 3.77-3.69 (m, 4H), 3.39-3.30 (m, 4H), 1.65 (d, J=6.8 Hz, 3H).

### Example 463. Synthesis of (2-chloro-4-((1-(3-(1,1-difluoro-2-hydroxyethyl)phenyl)-2-fluoroethyl)amino)-5,7-dihydro-6H-pyrrolo[3,4-d]pyrimidin-6-yl)(morpholino)methanone

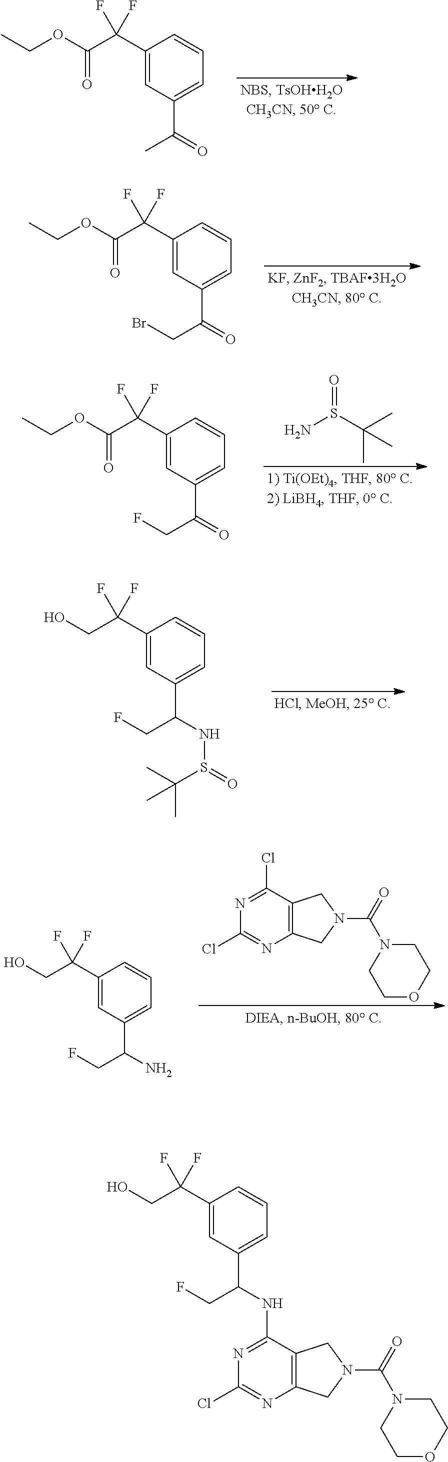

### Step 1.

To a solution of ethyl 2-(3-acetylphenyl)-2,2-difluoro-acetate (14.0 g, 57.8 mmol) in MeCN (140 mL) were added NBS (10.3 g, 57.8 mmol) and TsOH.H₂O (11.0 g, 57.8 mmol). The reaction was stirred at 50° C. for 15 h, then the mixture was added to ice water and neutralized with NaHCO₃. The mixture was extracted with EtOAc, dried over Na₂SO₄, filtered, and concentrated under reduced pressure. The crude residue was purified by column chromatography to give ethyl 2-[3-(2-bromoacetyl) phenyl]-2,2-difluoro-acetate (8.0 g, 43% yield). ¹H NMR (400 MHz, CHLOROFORM-d) δ ppm 8.23 (s, 1H), 8.14 (d, J=7.6 Hz, 1H), 7.87 (d, J=7.6 Hz, 1H), 7.63 (t, J=7.6 Hz, 1H), 4.47 (s, 2H), 4.33 (q, J=7.2 Hz, 2H), 1.33 (t, J=7.2 Hz, 3H).

### Step 2.

A solution of KF (452 mg, 7.79 mmol), TBAF.3H₂O (3.68 g, 11.7 mmol) and ZnF₂ (1.61 g, 15.6 mmol) in MeCN (50 mL) was stirred at 80° C. for 1 h, then ethyl 2-[3-(2-bromoacetyl)phenyl]-2,2-difluoro-acetate (5.0 g, 16 mmol) was added, and the mixture was stirred at 80° C. for 10 h. The reaction mixture was then added to ice water, extracted with EtOAc, dried over Na₂SO₄ and concentrated under reduced pressure. The crude residue was purified by column chromatography to give ethyl 2,2-difluoro-2-[3-(2-fluoroacetyl)phenyl] acetate (2.3 g, 57% yield). ¹H NMR (400 MHz, CHLOROFORM-d) δ ppm 8.15 (s, 1H), 8.06 (d, J=7.6 Hz, 1H), 7.89 (d, J=7.6 Hz, 1H), 7.64 (t, J=8.0 Hz, 1H), 5.54 (d, J=46.8 Hz, 2H), 4.33 (q, J=7.2 Hz, 2H), 1.33 (t, J=7.2 Hz, 3H).

### Step 3.

To a solution of ethyl 2,2-difluoro-2-[3-(2-fluoroacetyl)phenyl]acetate (0.50 g, 1.9 mmol) and 2-methylpropane-2-sulfinamide (256 mg, 2.11 mmol) in THF (5 mL) was added Ti(OEt)₄ (1.20 mL, 5.76 mmol). The mixture was stirred at 80° C. for 10 h, then cooled to 0° C. MeOH (77.8 μL, 1.92 mmol) and LiBH₄ (83.7 mg, 3.84 mmol) were added and the mixture was stirred at 0° C. for 1 h. The reaction mixture was added to ice water, filtered, dried over Na₂SO₄, and concentrated under reduced pressure. The crude product was purified by prep-TLC to give N-[1-[3-(1,1-difluoro-2-hydroxy-ethyl)phenyl]-2-fluoro-ethyl]-2-methyl-propane-2-sulfinamide (0.30 g, 48% yield). LCMS (ESI): m/z: [M+H] calculated for C₁₄H₂₁F₃NO₂S: 324.1. found 324.1.

### Step 4.

To a mixture of N-[1-[3-(1,1-difluoro-2-hydroxy-ethyl)phenyl]-2-fluoro-ethyl]-2-methyl-propane-2-sulfinamide (100 mg, 309 μmol) in MeOH (1 mL) was added a 4M solution of HCl in MeOH (232 μL, 928 μmol). The mixture was stirred at 25° C. for 2 h, then treated with a 1M solution of NaOH in MeOH to adjust to pH=8, and concentrated under reduced pressure to give 2-[3-(1-amino-2-fluoro-ethyl) phenyl]-2, 2-difluoro-ethanol (55 mg, crude). LCMS (ESI): m/z: [M+H] calculated for C₁₀H₁₃F₃NO: 220.0. found 220.1.

### Step 5.

To a solution of 2-[3-(1-amino-2-fluoro-ethyl) phenyl]-2,2-difluoro-ethanol (50 mg, 230 μmol) and (2,4-dichloro-5,7-dihydropyrrolo[3,4-d]pyrimidin-6-yl)-morpholino-methanone (69.2 mg, 228 μmol) in n-BuOH (1 mL) was added DIEA (79.5 μL, 456 μmol). The reaction was stirred at 80° C. for 5 h, then was filtered, concentrated under reduced pressure, and purified by prep-HPLC to give [2-chloro-4-[[1-[3-(1,1-difluoro-2-hydroxy-ethyl)phenyl]-2-fluoro-ethyl]amino]-5,7-dihydropyrrolo[3,4-d]pyrimidin-6-yl]-morpholino-methanone (20 mg, 18% yield). LCMS (ESI): m/z: [M+H] calculated for C₂₁H₂₄ClF₃N₅O₃: 486.1. found 486.1. ¹H NMR (400 MHz, METHANOL-d₄) δ ppm 7.63 (s, 1H), 7.56 (s, 1H), 7.52-7.44 (m, 2H), 5.76-5.64 (m, 1H), 4.78 (d, J=6.4 Hz, 1H), 4.67 (s, 2H), 4.66 (s, 1H), 4.59 (s, 2H), 3.89 (t, J=13.2 Hz, 2H), 3.77-3.68 (m, 4H), 3.40-3.34 (m, 4H).

### Examples 464 and 465. Synthesis of (2-chloro-4-(((R)-1-((R)-2-(hydroxymethyl)-2,3-dihydro-1H-inden-4-yl)ethyl)amino)-5,7-dihydro-6H-pyrrolo[3,4-d]pyrimidin-6-yl)(morpholino)methanone and (2-chloro-4-(((R)-1-((S)-2-(hydroxymethyl)-2,3-dihydro-1H-inden-4-yl)ethyl)amino)-5,7-dihydro-6H-pyrrolo[3,4-d]pyrimidin-6-yl)(morpholino)methanone

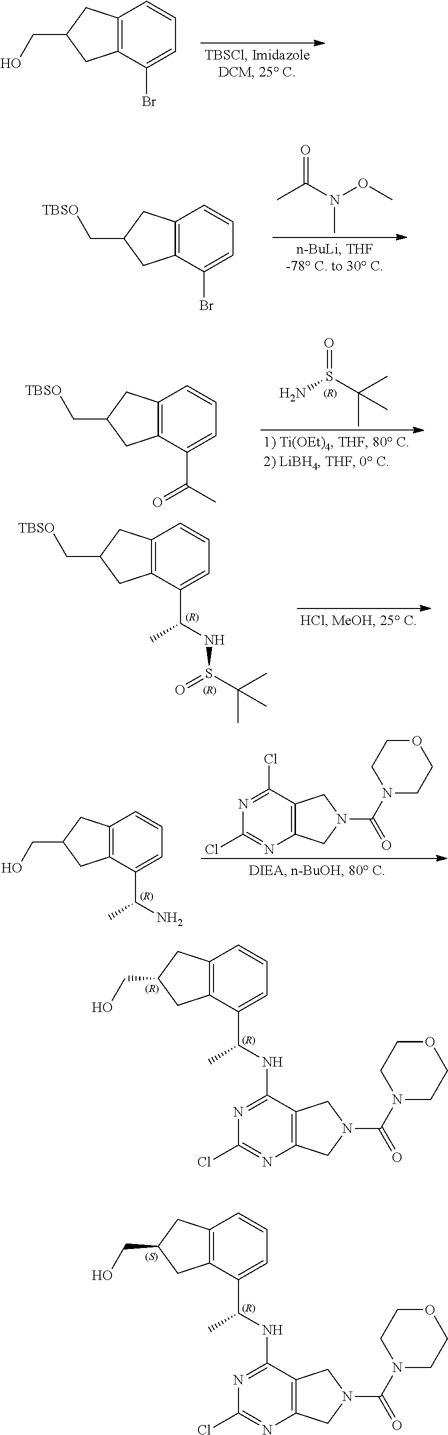

### Step 1.

To a mixture of (4-bromoindan-2-yl)methanol (900 mg, 3.96 mmol) in DCM (3 mL) were added imidazole (1.08 g, 15.6 mmol) and TBSCl (1.19 g, 7.93 mmol) and the mixture was stirred at 25° C. for 90 min. The reaction was then quenched with H₂O, extracted with EtOAc, dried over Na₂SO₄ and concentrated under reduced pressure. The crude residue was purified by prep-TLC to give (4-bromoindan-2-yl)methoxy-tert-butyl-dimethyl-silane (990 mg, 73% yield). ¹H NMR (400 MHz, METHANOL-d₄) δ ppm 7.25 (d, J=7.8, 1H), 7.12 (m, 1H), 7.01 (m, 1H), 3.64-3.55 (m, 2H), 3.15-3.07 (m, 1H), 3.05-2.95 (m, 1H), 2.71-2.60 (m, 3H), 0.88 (s, 9H), 0.05 (s, 6H).

### Step 2.

To a mixture of (4-bromoindan-2-yl)methoxy-tert-butyl-dimethyl-silane (990 mg, 2.90 mmol) in THF (2 mL) was added a 2.5 M solution of n-BuLi in hexanes (2.32 mL, 5.80 mmol) at −78° C. under N₂, and the mixture was stirred at −78° C. for 2 h. A solution of N-methoxy-N-methylacetamide (449 mg, 4.35 mmol) in THF (2 mL) was then added at −30° C. under N₂ and the mixture was stirred at −30° C. for 1 h. The reaction was quenched by the addition of H₂O, then extracted with EtOAc, dried over Na₂SO₄, and concentrated under reduced pressure. The crude residue was purified by prep-TLC to give 1-[2-[[tert-butyl(dimethyl)silyl]oxymethyl]indan-4-yl]ethanone (290 mg, 33% yield). ¹H NMR (400 MHz, METHANOL-d₄) δ ppm 7.70 (d, J=7.7 Hz, 1H), 7.36 (d, J=7.3 Hz, 1H), 7.22 (t, J=7.6 Hz, 1H), 3.54 (d, J=6.5 Hz, 2H), 3.23 (d, J=8.2 Hz, 1H), 3.05-2.95 (m, 2H), 2.75-2.68 (m, 1H), 2.62-2.54 (m, 1H), 2.51 (s, 3H), 0.87 (s, 9H), 0.04 (s, 6H).

### Step 3.

To a mixture of 1-[2-[[tert-butyl(dimethyl)silyl]oxymethyl]indan-4-yl]ethanone (270 mg, 887 μmol) in THF (2 mL) were added Ti(OEt)₄ (607 mg, 2.66 mmol) and (R)-2-methylpropane-2-sulfinamide (215 mg, 1.77 mmol). The mixture was heated to 80° C. and stirred for 7 h. The mixture was cooled to −4° C., then MeOH (35.9 μL, 887 μmol) and LiBH₄ (58.0 mg, 2.66 mmol) were added and the reaction was stirred at 0° C. for 1 h. The mixture was poured slowly into 1:1 H₂O/THF, then filtered over Celite®, washed with THF, and the filtrate evaporated under reduced pressure. The crude residue was purified by prep-TLC to give (R)—N-((1R)-1-(2-(((tert-butyldimethylsilyl)oxy)methyl)-2,3-dihydro-1H-inden-4-yl)ethyl)-2-methylpropane-2-sulfinamide (200 mg, 55% yield). ¹H NMR (400 MHz, METHANOL-d₄) δ ppm 7.21 (d, J=7.6 Hz, 1H), 7.15-7.08 (m, 2H), 4.60-4.56 (m, 1H), 3.64-3.59 (m, 2H), 3.01-2.95 (m, 2H), 2.85-2.61 (m, 3H), 1.48 (d, J=6.7 Hz, 3H), 1.21 (s, 9H), 0.91 (s, 9H), 0.07 (s, 6H).

### Step 4.

To a mixture of (R)—N-((1R)-1-(2-(((tert-butyldimethylsilyl)oxy)methyl)-2,3-dihydro-1H-inden-4-yl)ethyl)-2-methylpropane-2-sulfinamide (200 mg, 488 μmol) in MeOH (3 mL) was added a 4M solution of HCl in MeOH (1.22 mL, 4.88 mmol). The mixture was stirred at 25° C. for 2 h, then filtered and concentrated under reduced pressure to give (4-((R)-1-aminoethyl)-2,3-dihydro-1H-inden-2-yl)methanol (93 mg, crude). LCMS (ESI): m/z: [M+H] calculated for C₁₂H₁₈NO: 192.0. found 192.0.

### Step 5.

To a mixture of (4-((R)-1-aminoethyl)-2,3-dihydro-1H-inden-2-yl)methanol (40 mg, 87 μmol, 33% yield) in t-BuOH (2 mL) were added DIEA (60.1 μL, 345 μmol) and (2,4-dichloro-5,7-dihydropyrrolo[3,4-d]pyrimidin-6-yl)-morpholino-methanone (34.9 mg, 115 μmol) at 25° C. The mixture was stirred at 80° C. for 3 h, then concentrated under reduced pressure. The crude residue was purified by prep-HPLC to give [2-chloro-4-[[(1R)-1-[(2R)-2-(hydroxymethyl)indan-4-yl]ethyl]amino]-5,7-dihydropyrrolo[3,4-d]pyrimidin-6-yl]-morpholino-methanone (12.6 mg, 32% yield,) and [2-chloro-4-[[(1R)-1-[(2S)-2-(hydroxymethyl)indan-4-yl]ethyl]amino]-5,7-dihydropyrrolo [3,4-d]pyrimidin-6-yl]-morpholino-methanone (5.5 mg, 14% yield).

Data for [2-chloro-4-[[(1R)-1-[(2R)-2-(hydroxymethyl)indan-4-yl]ethyl]amino]-5,7-dihy dropyrrolo[3,4-d]pyrimidin-6-yl]-morpholino-methanone:

LCMS (ESI): m/z: [M+H] calculated for C₂₃H₂₉ClN₅O₃: 458.2. found 458.2; ¹H NMR (400 MHz, METHANOL-d₄) δ ppm 7.17-7.06 (m, 3H), 5.36 (d, J=7.1 Hz, 1H), 4.60-4.52 (m, 4H), 3.74-3.69 (m, 4H), 3.64-3.59 (m, 1H), 3.53 (d, J=7.5 Hz, 1H), 3.36-3.32 (m, 4H), 3.10-3.02 (m, 2H), 2.97-2.91 (m, 1H), 2.78 (d, J=5.5 Hz, 1H), 2.71-2.63 (m, 1H), 1.53 (d, J=6.8 Hz, 3H).

Data for [2-chloro-4-[[(1R)-1-[(2S)-2-(hydroxymethyl)indan-4-yl]ethyl]amino]-5,7-dihydropyrrolo[3,4-d]pyrimidin-6-yl]-morpholino-methanone:

LCMS (ESI): m/z: [M+H] calculated for C₂₃H₂₉ClN₅O₃: 458.2. found 458.2; ¹H NMR (400 MHz, METHANOL-d₄) δ ppm 7.16-7.07 (m, 3H), 5.38-5.30 (m, 1H), 4.59-4.55 (m, 4H), 3.69-3.73 (m, 4H), 3.56 (d, J=6.4 Hz, 2H), 3.34 (d, J=5.1 Hz, 4H), 3.05-2.95 (m, 2H), 2.77-2.69 (m, 3H), 1.52 (d, J=7.1 Hz, 3H).

### Example 466. Synthesis of [2-chloro-4-[[(1R)-1-(3,3-difluoro-2H-benzofuran-7-yl)ethyl]amino]-5,7-dihydropyrrolo[3,4-d]pyrimidin-6-yl]-morpholino-methanone

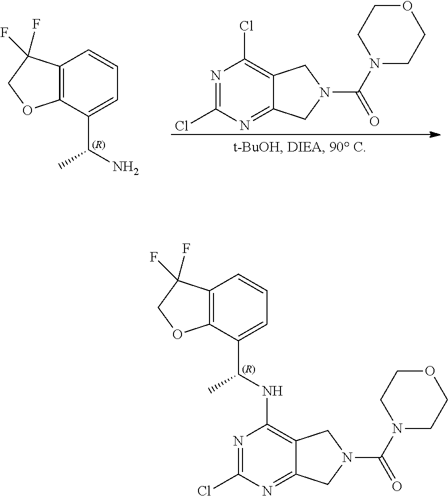

### Step 1.

To a solution of (2,4-dichloro-5,7-dihydropyrrolo[3,4-d]pyrimidin-6-yl)-morpholino-methanone (65.0 mg, 214 μmol) in t-BuOH (1.5 mL) were added (1R)-1-(3,3-difluoro-2H-benzofuran-7-yl)ethanamine hydrochloride (58.4 mg, 248 μmol) and DIEA (187 μL, 1.07 mmol). The reaction mixture was stirred at 90° C. for 1 h, then concentrated under reduced pressure. The crude residue was purified by prep-HPLC to give [2-chloro-4-[[(1R)-1-(3,3-difluoro-2H-benzofuran-7-yl)ethyl]amino]-5,7-dihydropyrrolo[3,4-d]pyrimidin-6-yl]-morpholino-methanone (23 mg, 23% yield). LCMS (ESI): m/z: [M+H] calculated for C₂₁H₂₃ClF₂N₅O₃: 466.1. found 466.1; H NMR (400 MHz, METHANOL-d₄) δ ppm 7.50-7.45 (m, 1H), 7.45-7.41 (m, 1H), 7.09-7.03 (m, 1H), 5.54-5.47 (m, 1H), 4.72 (t, J=16.1 Hz, 2H), 4.66-4.54 (m, 4H), 3.76-3.68 (m, 4H), 3.39-3.33 (m, 4H), 1.58 (d, J=7.1 Hz, 3H).

### Example 467. Synthesis of [2-chloro-4-[[(1R)-1-[3-(1,1-difluoroethyl)phenyl]ethyl]amino]-5,7-dihydropyrrolo[3,4-d]pyrimidin-6-yl]-morpholino-methanone

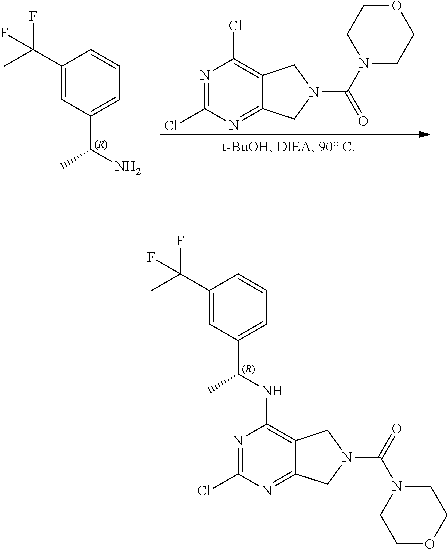

### Step 1.

To a solution of (1R)-1-[3-(1,1-difluoroethyl)phenyl]ethanamine (60.0 mg, 324 μmol) and (2,4-dichloro-5,7-dihydropyrrolo[3,4-d]pyrimidin-6-yl)-morpholino-methanone (98.2 mg, 324 μmol) in t-BuOH (2 mL) was added DIEA (169 μL, 972 μmol). The mixture was stirred at 90° C. for 1 h, then was added to ice water, extracted with EtOAc, dried over Na₂SO₄, and concentrated under reduced pressure. The crude residue was purified by prep-HPLC to give [2-chloro-4-[[(1R)-1-[3-(1,1-difluoroethyl)phenyl]ethyl]amino]-5,7-dihydropyrrolo[3,4-d]pyrimidin-6-yl]-morpholino-methanone (60 mg, 40% yield). LCMS (ESI): m/z: [M+H] calculated for C₂₁H₂₅ClF₂N₅O₂: 452.2. found: 451.9; ¹H NMR (400 MHz, METHANOL-d₄) δ ppm 7.58 (s, 1H), 7.54-7.46 (m, 1H), 7.45-7.37 (m, 2H), 5.41 (q, J=6.4 Hz, 1H), 4.61 (s, 2H), 4.57 (s, 2H), 3.75-3.68 (s, 4H), 3.38-3.30 (s, 4H), 1.90 (t, J=18.4 Hz, 3H), 1.58 (d, J=7.2 Hz, 3H).

### Example 468. Synthesis of [2-chloro-4-[[(1R)-1-(3-fluorobenzofuran-7-yl)ethyl]amino]-5,7-dihydropyrrolo[3,4-d]pyrimidin-6-yl]-morpholino-methanone

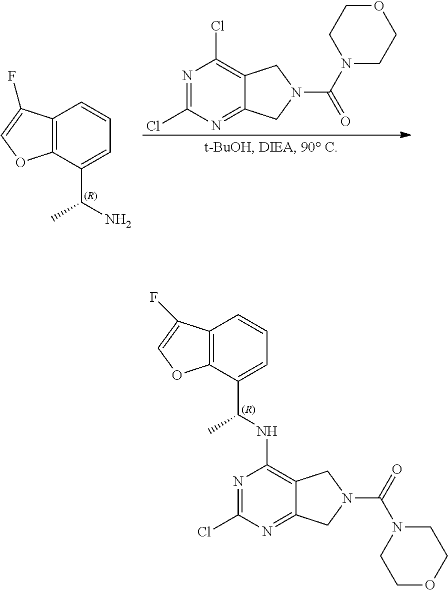

### Step 1.

A mixture of (1R)-1-(3-fluorobenzofuran-7-yl)ethanamine (50.0 mg, 279 μmol), (2,4-dichloro-5,7-dihydropyrrolo[3,4-d]pyrimidin-6-yl)-morpholino-methanone (102 mg, 335 μmol), and DIEA (243 μL, 1.40 mmol) in t-BuOH (1 mL) was sparged with N₂ and then was stirred at 90° C. for 15 h. The mixture was then concentrated under reduced pressure and the crude residue was purified by prep-HPLC to give [2-chloro-4-[[(1R)-1-(3-fluorobenzofuran-7-yl)ethyl]amino]-5,7-dihydropyrrolo[3,4-d]pyrimidin-6-yl]-morpholino-methanone (22.6 mg, 17% yield). LCMS (ESI): m/z: [M+H] calculated for C₂₁H₂₂ClFN₅O₃: 446.1. found 446.1; ¹H NMR (400 MHz, METHANOL-d₄) δ ppm 7.86 (d, J=4.0 Hz, 1H) 7.49 (d, J=8.0 Hz, 1H) 7.34 (d, J=8.0 Hz, 1H) 7.29-7.22 (m, 1H) 5.80 (q, J=8.0 Hz, 1H) 4.77-4.57 (m, 4H), 3.74-3.69 (m, 4H) 3.37-3.31 (m, 4H) 1.66 (d, J=8.0 Hz, 3H).

### Example 469. Synthesis of [2-chloro-4-[[(1R)-1-[3-[2-(cyclopropoxy)-1,1-difluoro-ethyl]-2-fluoro-phenyl]ethyl]amino]-5,7-dihydropyrrolo[3,4-d]pyrimidin-6-yl]-morpholino-methanone

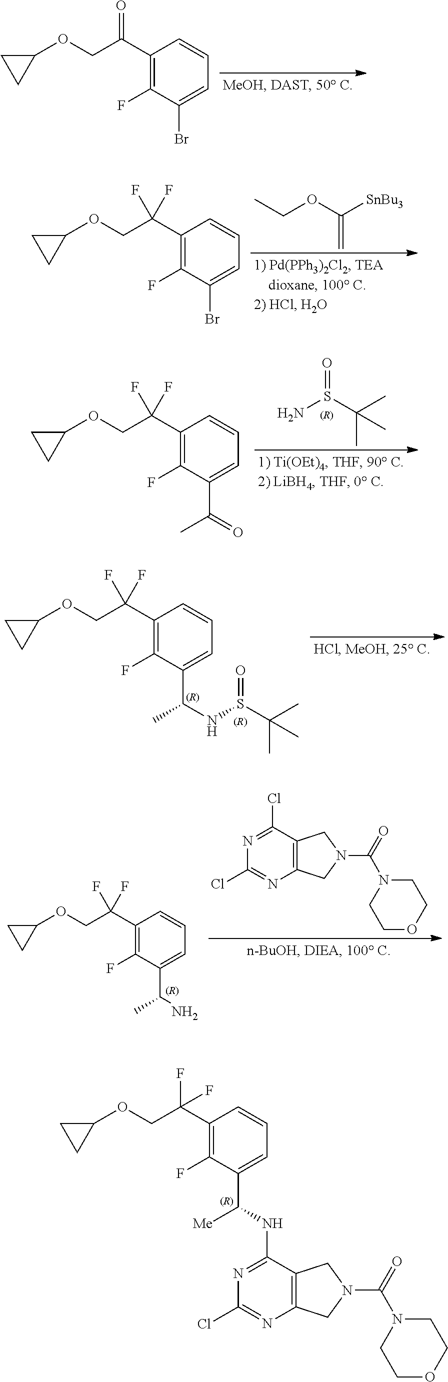

### Step 1.

To a solution of 1-(3-bromo-2-fluoro-phenyl)-2-(cyclopropoxy) ethanone (1.85 g, 6.77 mmol) in DAST (20 mL) was added MeOH (2.74 μL, 67.7 μmol) and the reaction was stirred at 50° C. for 12 h. The mixture was then poured into water and extracted with EtOAc. The combined organic extracts were washed with brine, dried over Na₂SO₄, filtered and concentrated under reduced pressure. The crude residue was then purified by column chromatography to give bromo-3-[2-(cyclopropoxy)-1, 1-difluoro-ethyl]-2-fluoro-benzene (1.5 g, 75% yield). ¹H NMR (400 MHz, METHANOL-d₄) δ ppm 7.67 (t, J=7 Hz, 1H), 7.52 (t, J=7.2 Hz, 1H), 7.15-7.08 (m, 1H), 4.06 (t, J=13.5 Hz, 2H), 3.46-3.42 (m, 1H), 0.60-0.54 (m, 2H), 0.51-0.45 (m, 2H).

### Step 2.

To a mixture of 1-bromo-3-[2-(cyclopropoxy)-1,1-difluoro-ethyl]-2-fluoro-benzene (500 mg, 1.69 mmol) and tributyl (1-ethoxyvinyl) stannane (858 μL, 2.54 mmol) in dioxane (5 mL) were added TEA (590 μL, 4.24 mmol) and Pd(PPh₃)₂Cl₂ (119 mg, 169 μmol). The mixture was sparged with argon gas then stirred at 100° C. for 12 h. After cooling to rt a 2M solution of aqueous HCl (10 mL, 20 mmol) was added and the mixture was stirred for 1 h (pH=2). The reaction was then extracted with EtOAc and the combined organic extracts were treated with an aqueous KF solution (20 ml, 5 g KF) and stirred for 2 h. The mixture was filtered and the solution was washed with brine, dried over Na₂SO₄, filtered and concentrated under reduced pressure. The crude residue was purified by column chromatography to give 1-[3-[2-(cyclopropoxy)-1,1-difluoro-ethyl]-2-fluoro-phenyl]ethanone (360 mg, 82% yield). ¹H NMR (400 MHz, CHLOROFORM-d) δ ppm 7.89 (t, J=6.8 Hz, 1H), 7.67 (t, J=6.8 Hz, 1H), 7.25-7.18 (m, 1H), 4.01 (t, J=13.4 Hz, 2H), 3.38-3.34 (m, 1H), 2.60-2.58 (m, 3H), 0.53-0.35 (m, 4H).

### Step 3.

To a mixture of 1-[3-[2-(cyclopropoxy)-1,1-difluoro-ethyl]-2-fluoro-phenyl]ethanone (360 mg, 1.39 mmol) and (R)-(+)-2-Methyl-2-propanesulfinamide (253 mg, 2.09 mmol) in THF (5 mL) was added Ti(OEt)₄ (1.45 mL, 6.97 mmol). The mixture was heated to 90° C. and stirred for 2 h. The mixture was cooled to 0° C. MeOH (56.4 μL, 1.39 mmol) and LiBH₄ (33.4 mg, 1.53 mmol) were added and the mixture was stirred at 0° C. for 30 min. The reaction was poured into 1:1 H₂O/THF then filtered over Celite®, and concentrated under reduced pressure. The crude residue was purified by column chromatography to give (R)—N—((R)-1-(3-(2-cyclopropoxy-1,1-difluoroethyl)-2-fluorophenyl)ethyl)-2-methylpropane-2-sulfinamide (170 mg, 34% yield). ¹H NMR (400 MHz, CHLOROFORM-d) δ ppm 7.53-−7.48 (m, 2H), 7.22 (t, J=7.6 Hz, 1H), 4.88-4.86 (m, 1H), 4.05 (t, J=13.6 Hz, 2H), 3.52 (br d, J=4.8 Hz, 1H), 3.44 (m, 1H), 1.54 (d, J=6.4 Hz, 3H), 1.23 (s, 9H), 0.60-0.54 (m, 2H), 0.50-0.44 (m, 2H).

### Step 4.

To a mixture of (R)—N—((R)-1-(3-(2-cyclopropoxy-1,1-difluoroethyl)-2-fluorophenyl)ethyl)-2-methylpropane-2-sulfinamide (170 mg, 467 μmol) in MeOH (2 mL) was added a 4M solution of HCl in MeOH (468 μL, 1.87 mmol). The reaction was stirred at 25° C. for 30 min. The mixture was then concentrated under reduced pressure, treated dropwise with NaOH in MeOH until pH=7, then concentrated under reduced pressure again. The residue was dissolved in 10:1 DCM:MeOH and filtered. The filtrate was concentrated under reduced pressure to give (1R)-1-[3-[2-(cyclopropoxy)-1,1-difluoro-ethyl]-2-fluoro-phenyl]ethanamine (121 mg, crude).

### Step 5.

To a mixture of (1R)-1-[3-[2-(cyclopropoxy)-1,1-difluoro-ethyl]-2-fluoro-phenyl]ethanamine (121 mg, 467 μmol) and (2,4-dichloro-5,7-dihydropyrrolo[3,4-d]pyrimidin-6-yl)-morpholino-methanone (212 mg, 700 μmol) in n-BuOH (2 mL) was added DIEA (406 μL, 2.33 mmol). The reaction was stirred at 100° C. for 12 h. The mixture was then filtered and the filtrate was purified by prep-HPLC to give [2-chloro-4-[[(1R)-1-[3-[2-(cyclopropoxy)-1,1-difluoro-ethyl]-2-fluoro-phenyl]ethyl]amino]-5,7-dihydropyrrolo[3,4-d]pyrimidin-6-yl]-morpholino-methanone (72 mg, 29% yield over 2 steps). LCMS (ESI): m/z: [M+H] calculated for C₂₄H₂₈ClF₃N₅O₃: 526.2. found 526.4. ¹H NMR (400 MHz, METHANOL-d4) δ ppm 7.54 (t, J=6.8 Hz, 1H), 7.44 (t, J=6.6 Hz, 1H), 7.22 (t, J=7.6 Hz, 1H), 5.59 (q, J=7.2 Hz, 1H), 4.65 (br s, 2H), 4.60-4.55 (m, 2H), 4.06 (t, J=13.4 Hz, 2H), 3.75-3.69 (m, 4H), 3.42-3.38 (m, 1H), 3.38-3.34 (m, 4H), 1.59 (d, J=6.8 Hz, 3H), 0.39-0.44 (m, 4H).

### Example 470. Synthesis of N-{1-[3-amino-5-(trifluoromethyl)phenyl]propyl}-2-chloro-6-(morpholine-4-carbonyl)-5H,6H,7H-pyrrolo[3,4-d]pyrimidin-4-amine

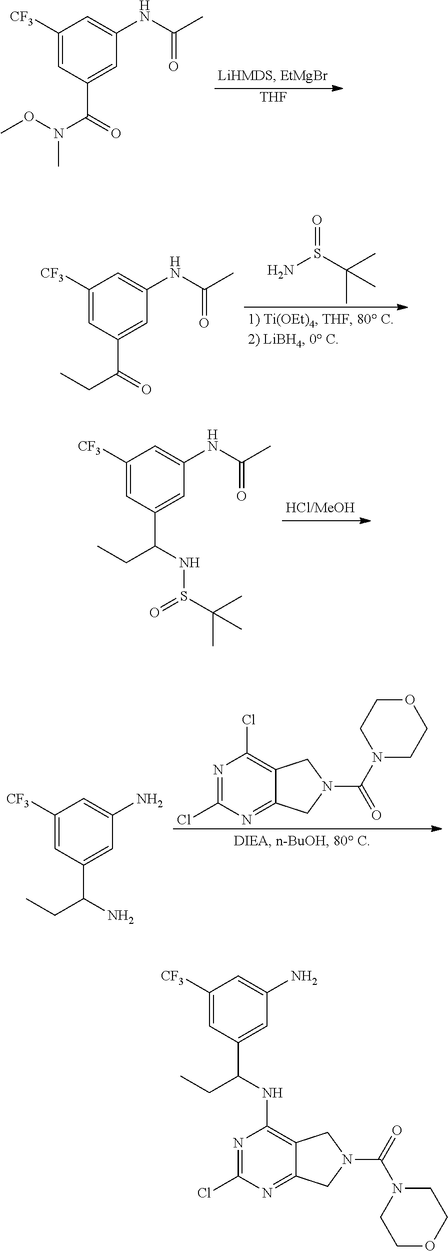

### Step 1.

To a solution of 3-acetamido-N-methoxy-N-methyl-5-(trifluoromethyl)benzamide (5 g, 17.23 mmol) in THF (35 mL) was added LiHMDS (1 M, 17.23 mL) at 0° C., the reaction mixture was stirred at 0° C. for 0.5 h, and then EtMgBr (2 M, 30.15 mL) was added at 0° C. The reaction mixture was stirred at 25° C. for 0.5 h and then poured over ice and adjusted to pH ˜ 4 with 2 M HCl. After extraction with EtOAc the combined organic phase was washed with brine and dried over Na₂SO₄. The solvent was removed under reduced pressure and the residue was purified by column chromatography to give N-[3-propanoyl-5-(trifluoromethyl)phenyl]acetamide (3.3 g, 73.9% yield) as a light yellow solid. ¹H NMR (400 MHz, CHLOROFORM-d) δ ppm 8.22 (s, 1H) 8.19 (s, 1H) 7.97 (br s, 1H) 7.91 (s, 1H) 3.03 (q, J=7.2 Hz, 2H) 2.25 (s, 3H) 1.24 (t, J=7.2 Hz, 3H).

### Step 2.

A mixture of N-[3-propanoyl-5-(trifluoromethyl)phenyl]acetamide (3.3 g, 12.73 mmol), 2-methylpropane-2-sulfinamide (3.09 g, 25.46 mmol) in THF (30 mL) was added Ti(OEt)₄ (8.71 g, 38.19 mmol, 7.92 mL) at 25° C. The mixture was stirred at 90° C. for 10 h. After cooling to 0° C., MeOH (407.90 mg, 12.73 mmol, 515.16 μL) was added, followed by LiBH₄ (277.31 mg, 12.73 mmol) and the resulting mixture was stirred at 0° C. for 1 h. H₂O was added the mixture was filtered. The filtrate was extracted with EtOAc and the combined organic layers were washed with brine and dried over Na₂SO₄. The solvent was removed under reduced pressure and the crude residue was purified by prep-HPLC to give N-[3-[1-(tert-butylsulfinylamino)propyl]-5-(trifluoromethyl)phenyl]acetamide (2.5 g, 53.89% yield) as a yellow solid. ¹H NMR (400 MHz, METHANOL-d₄) δ ppm 7.89 (s, 1H) 7.73 (s, 1H) 7.43 (s, 1H) 4.22 (t, J=7.2 Hz, 1H) 2.15 (s, 3H) 1.97 (m, 1H) 1.80 (m, 1H) 1.23 (s, 9H) 0.92 (t, J=7.4 Hz, 3H).

### Step 3.

A solution of N-[3-[1-(tert-butylsulfinylamino)propyl]-5-(trifluoromethyl)phenyl]acetamide (500 mg, 1.37 mmol) in HCl/MeOH (4 M, 10 mL) was stirred at 25° C. for 3 h. The reaction mixture was concentrated under reduced pressure and the crude residue was diluted with MeOH (2 mL) and adjusted to pH ˜ 8 with aqueous NaOH. Removal of the solvent under reduced pressure gave 3-(1-aminopropyl)-5-(trifluoromethyl)aniline (290 mg, 96.9% yield) as a colorless oil. LCMS (ESI): m/z: [M+H] calculated for C₁₀H₁₄F₃N₂: 219.1. found 219.0.

### Step 4.

To a solution of (2,4-dichloro-5,7-dihydropyrrolo[3,4-d]pyrimidin-6-yl)-morpholino-methanone (100 mg, 329.88 μmol) and 3-(1-aminopropyl)-5-(trifluoromethyl)aniline (107.98 mg, 494.8 μmol) in n-BuOH (2 mL) was added DIEA (127.90 mg, 989.63 μmol, 172.38 μL). The mixture was stirred at 80° C. for 10 h. The solvent was removed under reduced pressure and the crude residue was purified by prep-HPLC to give [4-[l-[3-amino-5-(trifluoromethyl)phenyl]propylamino]-2-chloro-5,7-dihydropyrrolo[3,4-d]pyrimidin-6-yl]-morpholino-methanone (57.8 mg, 35.01% yield). LCMS (ESI): m/z: [M+H] calculated for C₂₁H₂₅ClF₃N₆O₂: 485.2. found 485.1; H NMR (400 MHz, METHANOL-d₄) δ ppm 6.92 (s, 1H) 6.89 (s, 1H) 6.81 (s, 1H) 5.09 (s, 1H) 4.62 (s, 2H) 4.57 (s, 2H) 3.72 (t, J=4.6 Hz, 4H) 3.35 (t, J=4.6 Hz, 4H) 1.95-1.84 (m, 2H) 0.97 (t, J=7.4 Hz, 3H).

### Example 471. Synthesis of 2-[3-(1-{[2-chloro-6-(4-methoxyoxane-4-carbonyl)-5H,6H,7H-pyrrolo[3,4-d]pyrimidin-4-yl]amino}ethyl)-2-fluorophenyl]-2,2-difluoroethan-1-ol

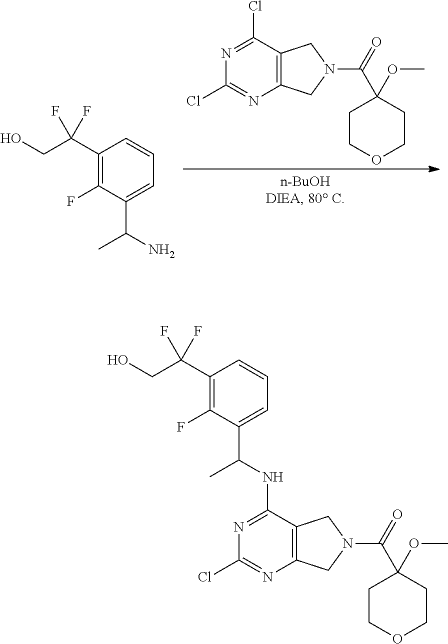 [SMILES: CC(N)c1cccc(C(F)(F)CO)c1F ; COC1(C(=O)N2Cc3nc(Cl)nc(Cl)c3C2)CCOCC1 ; COC1(C(=O)N2Cc3nc(Cl)nc(NC(C)c4cccc(C(F)(F)CO)c4F)c3C2)CCOCC1]

### Step 1.

To a solution of 2-[3-(1-aminoethyl)-2-fluoro-phenyl]-2,2-difluoro-ethanol hydrochloride (80 mg, 312.9 μmol) in n-BuOH (2 mL) was added DIEA (121.32 mg, 938.7 μmol, 163.5 μL) and (2,4-dichloro-5,7-dihydropyrrolo[3,4-d]pyrimidin-6-yl)-(4-methoxytetrahydropyran-4-yl)methanone (103.94 mg, 312.91 μmol). The mixture was stirred at 80° C. for 2h. The solvent was removed under reduced pressure and the crude residue was purified by prep-HPLC to give [2-chloro-4-[1-[3-(1,1-difluoro-2-hydroxy-ethyl)-2-fluoro-phenyl]ethylamino]-5,7-dihydropyrrolo[3,4-d]pyrimidin-6-yl]-(4-methoxytetrahydropyran-4-yl)methanone (60 mg, 37.2% yield). LCMS (ESI): m/z: [M+H] calculated for C₂₃H₂₇ClF₃N₄O₄: 515.2. found 515.2; ¹H NMR (400 MHz, METHANOL-d₄) δ=7.54-7.59 (m, 1H), 7.45-7.52 (m, 1H), 7.22-7.28 (m, 1H), 5.63-5.66 (m, 1H), 4.98-5.07 (s, 2H), 4.51-4.62 (m, 2H), 4.01-3.98 (m, 2H), 3.76-3.82 (m, 4H), 3.23-3.33 (m, 3H), 1.94-2.12 (m, 4H), 1.60 (d, J=6.8 Hz, 3H).

### Example 472. Synthesis of 2-chloro-6-(morpholine-4-carbonyl)-N-{1-[4-(trifluoromethyl)pyridin-2-yl]ethyl}-5H,6H,7H-pyrrolo[3,4-d]pyrimidin-4-amine

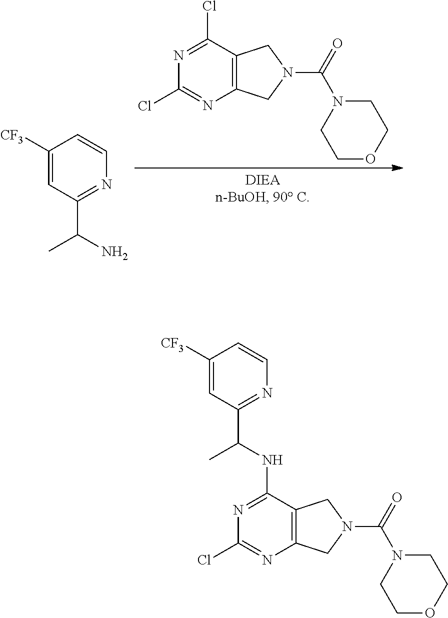 [SMILES: CC(N)c1cc(C(F)(F)F)ccn1 ; O=C(N1CCOCC1)N1Cc2nc(Cl)nc(Cl)c2C1 ; CC(Nc1nc(Cl)nc2c1CN(C(=O)N1CCOCC1)C2)c1cc(C(F)(F)F)ccn1]

### Step 1.

To a solution of 1-[4-(trifluoromethyl)-2-pyridyl]ethanamine (100.00 mg, 525.86 μmol) and (2,4-dichloro-5,7-dihydropyrrolo[3,4-d]pyrimidin-6-yl)-morpholino-methanone (159.41 mg, 525.86 μmol) in n-BuOH (1 mL) was added DIEA (135.9 mg, 1.05 mmol, 183.19 μL). The mixture was stirred at 90° C. for 1 h. After cooling to rt the mixture was filtered and the filter cake was s purified by prep-HPLC to give [2-chloro-4-[1-[4-(trifluoromethyl)-2-pyridyl] ethylamino]-5, 7-dihydropyrrolo [3, 4-d] pyrimidin-6-yl]-morpholino-methanone (50 mg, 20.68% yield). LCMS (ESI): m/z: [M+H] calculated for C₁₉H₂₁ClF₃N₆O₂: 457.1. found 457.1; ¹H NMR (400 MHz, METHANOL-d₄) δ=8.75 (d, J=5.2 Hz, 1H), 7.73 (s, 1H), 7.56 (d, J=4.4 Hz, 1H), 5.45 (q, J=7.2 Hz, 1H), 4.67 (s, 2H), 4.58 (s, 2H), 3.76-3.70 (m, 4H), 3.39-3.34 (m, 4H), 1.63 (d, J=7.2 Hz, 3H).

### Example 473. Synthesis of 2-chloro-6-(morpholine-4-carbonyl)-N-[(1R)-1-[6-(trifluoromethyl)pyridin-2-yl]ethyl]-5H,6H,7H-pyrrolo[3,4-d]pyrimidin-4-amine

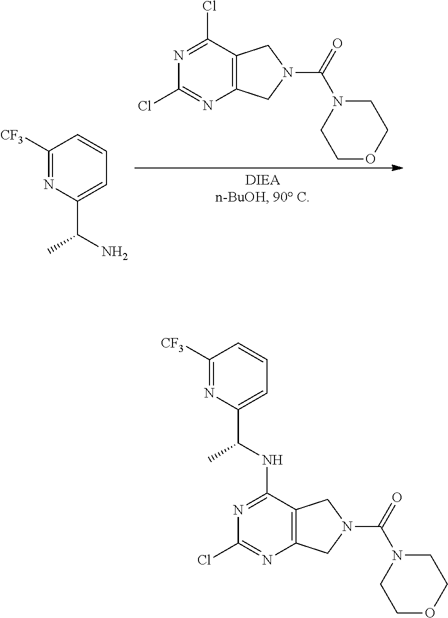 [SMILES: C[C@@H](N)c1cccc(C(F)(F)F)n1 ; O=C(N1CCOCC1)N1Cc2nc(Cl)nc(Cl)c2C1 ; C[C@@H](Nc1nc(Cl)nc2c1CN(C(=O)N1CCOCC1)C2)c1cccc(C(F)(F)F)n1]

### Step 1.

To a solution of (1R)-1-[6-(trifluoromethyl)-2-pyridyl]ethanamine (0.1 g, 525.86 μmol) and (2,4-dichloro-5,7-dihydropyrrolo[3,4-d]pyrimidin-6-yl)-morpholino-methanone (159.41 mg, 525.86 μmol) in n-BuOH (2 mL) was added DIEA (135.93 mg, 1.05 mmol, 183.19 μL). The mixture was stirred at 90° C. for 1 h and then poured into ice water. After extraction with DCM the combined organic phase was dried with Na₂SO₄ and the solvent was removed under reduced pressure. The crude residue was purified by prep-HPLC to give [2-chloro-4-[[(1R)-1-[6-(trifluoromethyl)-2-pyridyl]ethyl]amino]-5,7-dihydropyrrolo[3,4-d]pyrimidin-6-yl]-morpholino-methanone (80 mg, 23% yield). LCMS (ESI): m/z: [M+H] calculated for C₁₉H₂₁ClF₃N₆O₂: 457.1. found 457.0; ¹H NMR (400 MHz, CHLOROFORM-d) δ=7.91 (t, J=7.6 Hz, 1H), 7.63 (d, J=7.6 Hz, 1H), 7.54 (d, J=7.6 Hz, 1H), 6.29 (br d, J=7.2 Hz, 1H), 5.52 (t, J=6.8 Hz, 1H), 4.69-4.77 (m, 1H), 4.55-4.66 (m, 3H), 3.70-3.79 (m, 4H), 3.42-3.32 (m, 4H), 1.61 (d, J=6.8 Hz, 3H).

### Example 474. Synthesis of 2-chloro-6-(morpholine-4-carbonyl)-N-[(1R)-1-[2-(trifluoromethyl)-1,3-thiazol-5-yl]ethyl]-5H,6H,7H-pyrrolo[3,4-d]pyrimidin-4-amine

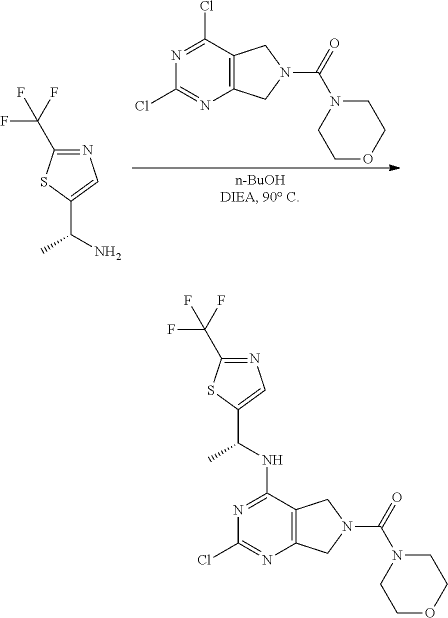 [SMILES: C[C@@H](N)c1cnc(C(F)(F)F)s1 ; O=C(N1CCOCC1)N1Cc2nc(Cl)nc(Cl)c2C1 ; C[C@@H](Nc1nc(Cl)nc2c1CN(C(=O)N1CCOCC1)C2)c1cnc(C(F)(F)F)s1]

### Step 1.

A mixture of (1R)-1-[2-(trifluoromethyl)thiazol-5-yl]ethanamine (120 mg, 611.64 μmol), (2,4-dichloro-5,7-dihydropyrrolo[3,4-d]pyrimidin-6-yl)-morpholino-methanone (185.41 mg, 611.64 μmol) and DIEA (395.25 mg, 3.06 mmol, 532.69 μL) in n-BuOH (4 mL) was degassed then stirred at 90° C. for 10 h under N₂. After cooling to rt the solvent was removed under reduced pressure and the crude residue was purified by prep-HPLC to give [2-chloro-4-[[(1R)-1-[2-(trifluoromethyl)thiazol-5-yl]ethyl]amino]-5,7-dihydropyrrolo[3,4-d]pyrimidin-6-yl]-morpholino-methanone (5.8 mg, 2.05% yield). LCMS (ESI): m/z: [M+H] calculated for C₁₇H₁₉ClF₃N₆O₂S: 463.09. found 462.8; ¹H NMR (400 MHz, METHANOL-d₄) δ=7.96 (s, 1H) 5.76 (q, J=7.00 Hz, 1H), 4.55-4.63 (m, 4H), 3.78-3.65 (m, 4H), 3.37-3.33 (m, 4H), 1.76 (d, J=7.0 Hz, 3H).

### Example 475. Synthesis of 2-chloro-N-[(1R)-1-{3-[difluoro(methoxy)methyl]phenyl}ethyl]-6-(morpholine-4-carbonyl)-5H,6H,7H-pyrrolo[3,4-d]pyrimidin-4-amine

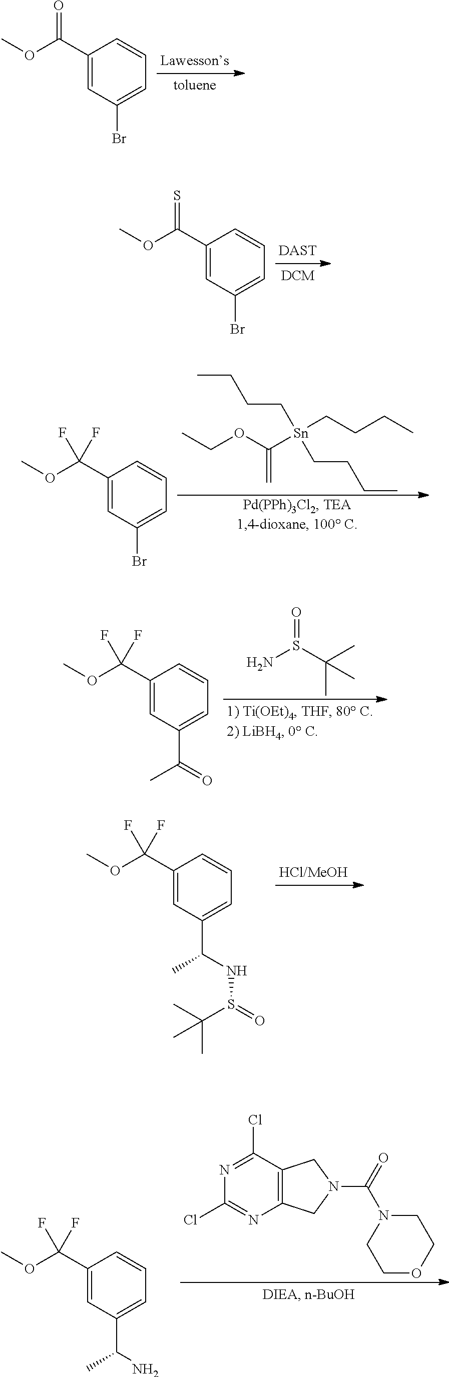

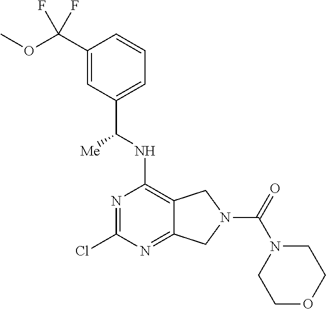 [SMILES: COC(F)(F)c1cccc([C@@H](C)Nc2nc(Cl)nc3c2CN(C(=O)N2CCOCC2)C3)c1]

### Step 1.

To a solution of methyl 3-bromobenzoate (10 g, 46.50 mmol) in toluene (10 mL) was added 2,4-bis(4-methoxyphenyl)-2,4-dithioxo-1,3,2,4dithiadiphosphetane (20.69 g, 51.15 mmol). The mixture was stirred in a microwave reactor at 140° C. for 4 h. After cooling to rt the solvent was removed under reduced pressure and the crude residue was purified by column chromatography to give O-methyl 3-bromobenzenecarbothioate (4 g, 37.22% yield). ¹H NMR (400 MHz, METHANOL-d₄) δ ppm 8.29 (t, J=1.76, 1H) 8.08-8.16 (m, 1H) 7.70-7.77 (m, 1H) 7.34 (t, J=7.94, 1H) 4.27-4.33 (m, 3H).

### Step 2.

To a solution of O-methyl 3-bromobenzenecarbothioate (3 g, 12.98 mmol) in DCM (14 mL) was added DAST (6.28 g, 38.94 mmol, 5.15 mL) and NBS (5.54 g, 31.15 mmol). The mixture was stirred at 25° C. for 16 h and then poured into aqueous bicarbonate. The aqueous phase was extracted with DCM and the combined organic phase was washed with brine and dried over Na₂SO₄. The solvent was removed under reduced pressure and the crude residue was purified by column chromatography to give 1-bromo-3-[difluoro(methoxy)methyl]benzene (2.5 g, 81.25% yield. ¹H NMR (400 MHz, METHANOL-d₄) δ ppm 7.71 (s, 1H) 7.62-7.66 (m, 1H) 7.54-7.57 (m, 1H) 7.33-7.40 (m, 1H) 3.69 (s, 3H).

### Step 3.

To a mixture of 1-bromo-3-[difluoro(methoxy)methyl]benzene (1 g, 4.22 mmol) and tributyl(1-ethoxyvinyl)stannane (2.29 g, 6.33 mmol, 2.14 mL) in dioxane (10 mL) was added TEA (1.07 g, 10.55 mmol, 1.47 mL) and Pd(PPh₃)₂Cl₂ (296.11 mg, 421.87 μmol). The mixture was sparged with N₂ and then stirred under N₂ at 100° C. for 3 h, after which the mixture was cooled to rt and aq. HCl (5 mL, 1 M) was added. The mixture stirred at rt for 1 h and then filtered. The filtrate was extracted with EtOAc and the combined organic layers were poured into aq. KF (20 mL, 5 g KF) and stirred for 2 h. The mixture was filtered and the organic layer was washed brine and dried over Na₂SO₄. The solvent was removed under reduced pressure and the solvent was removed under reduced pressure. The crude residue was purified by column chromatography to give 1-[3-[difluoro(methoxy)methyl]phenyl]ethanone (530 mg, 62.8% yield). ¹H NMR (400 MHz, METHANOL-d₄) δ ppm 8.17 (s, 1H) 8.11-8.13 (d, J=8, 1H) 7.81-7.83 (d, J=7.2, 1 H) 7.58-7.62 (m, J=8, 1H) 3.79 (s, 3H) 2.62 (s, 3H).

### Step 4.

To a solution of 1-[3-[difluoro(methoxy)methyl]phenyl]ethanone (530 mg, 2.65 mmol) and (R)-2-methylpropane-2-sulfinamide (481.34 mg, 3.97 mmol) in THF (5 mL) was added tetraethoxytitanium (3.02 g, 13.24 mmol, 2.75 mL) and the mixture was stirred at 90° C. for 3h. After cooling to 0° C. LiBH₄ (63.43 mg, 2.91 mmol) and MeOH (84.83 mg, 2.65 mmol, 107.13 μL) were added and the reaction was stirred at 0° C. for 30 min. The reaction mixture was then poured into water and the aqueous phase was extracted with EtOAc. The combined organic phase was washed with brine and dried with Na₂SO₄. The solvent was removed under reduced pressure and the crude residue was purified by column chromatography to give N-[(1R)-1-[3-[difluoro(methoxy)methyl]phenyl]ethyl]-2-methyl-propane-2-sulfinamide (640 mg, 60.95% yield). ¹H NMR (400 MHz, METHANOL-da₄) δ ppm 7.67 (s, 1H) 7.53-7.59 (m, 1H) 7.48-7.52 (m, 1H) 7.42-7.47 (m, 1H) 4.55 (q, J=6.72, 1H) 3.72 (s, 3H) 1.48-1.62 (m, 3H) 1.18-1.34 (m, 9H).

### Step 5.

To a solution of N-[(1R)-1-[3-[difluoro(methoxy)methyl]phenyl]ethyl]-2-methyl-propane-2-sulfinamide (200 mg, 654.92 μmol) in dioxane (2 mL) was added HCl/dioxane (4 M, 327.46 μL). The mixture was stirred at 25° C. for 2 h. The solvent was removed under reduced pressure to give (1R)-1-[3-[difluoro(methoxy)methyl]phenyl]ethanamine (131.78 mg, crude), which was used to the next step without purification. LCMS (ESI): m/z: [M+H] calculated for C₁₀H₁₄F₂NO: 202.1. found 202.2.

### Step 6.

To a solution of (1R)-1-[3-[difluoro(methoxy)methyl]phenyl]ethanamine (131 mg, 651.05 μmol) and (2,4-dichloro-5,7-dihydropyrrolo[3,4-d]pyrimidin-6-yl)-morpholino-methanone (197.36 mg, 651.05 μmol) in n-BuOH (2 mL) was added DIEA (252.43 mg, 1.95 mmol, 340.20 μL). The mixture was stirred at 100° C. for 3 h. After cooling to rt the solvent was removed under reduced pressure and the crude residue was purified by prep-HPLC to give [2-chloro-4-[[(1R)-1-[3-[difluoro(methoxy)methyl]phenyl]ethyl]amino]-5,7-dihydropyrrolo[3,4-d]pyrimidin-6-yl]-morpholino-methanone (30 mg, 9.61% yield). LCMS (ESI): m/z: [M+H] calculated for C₂₁H₂₅ClF₂N₅O₃: 468.2. found 468.2; ¹H NMR (400 MHz, MeOH-d6) δ ppm 7.64 (s, 1H) 7.51-7.53 (d, J=8 Hz, 1H) 7.45-7.47 (d, 1H) 7.38-7.42 (t, J=8 Hz, 1H) 5.38-5.42 (m, 1H) 4.54-4.59 (m, 4H) 3.69-3.72 (m, 4H) 3.68 (s, 3H) 3.30-3.34 (m, 4H) 1.56 (d, J=7.2, 3H).

### Example 476. Synthesis of 2-chloro-N-[1-(4,4-difluoro-3,4-dihydro-2H-1-benzopyran-8-yl)ethyl]-6-(morpholine-4-carbonyl)-5H,6H,7H-pyrrolo[3,4-d]pyrimidin-4-amine

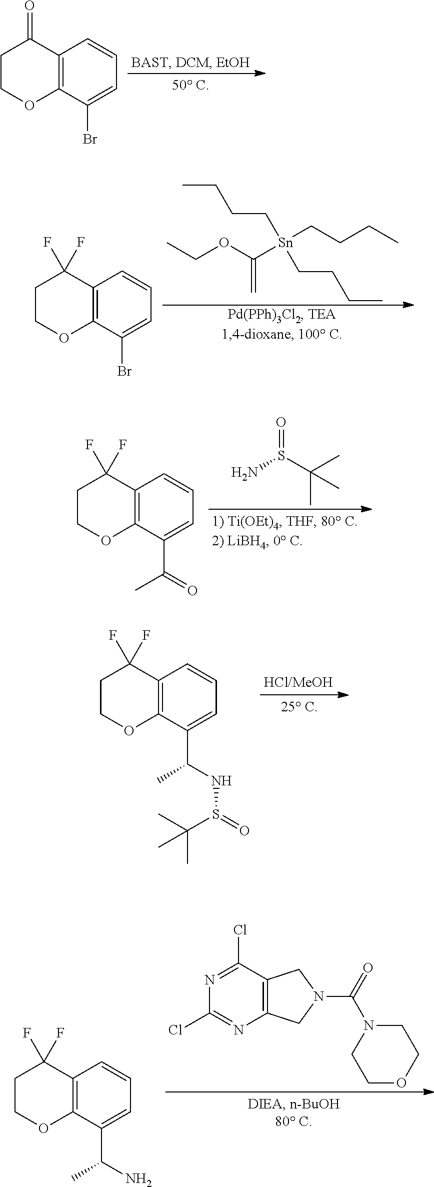

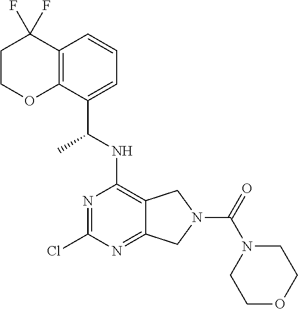 [SMILES: C[C@@H](Nc1nc(Cl)nc2c1CN(C(=O)N1CCOCC1)C2)c1cccc2c1OCCC2(F)F]

### Step 1.

To a solution of 8-bromochroman-4-one (1 g, 4.40 mmol) in DCM (5 mL) and EtOH (0.1 mL) was added BAST (9.74 g, 44.04 mmol, 9.65 mL). The mixture was stirred at 50° C. for 36 h. After cooling to rt aqueous NaHCO₃ was added and the mixture was extracted with EtOAc. The combined organic layers were washed with brine and dried over Na₂SO₄. The solvent was removed under reduced pressure and the crude residue was purified by column chromatography to give 8-bromo-4,4-difluoro-chromane (0.6 g, 54.70% yield). ¹H NMR (400 MHz, METHANOL-d₄) δ ppm 7.63 (d, J=8.0 Hz, 1H) 7.55 (d, J=7.2 Hz, 1H) 6.95 (t, J=8.0 Hz, 1H) 4.49-4.39 (m, 2H) 2.60-2.43 (m, 2H).

### Step 2.

To a mixture of 8-bromo-4,4-difluoro-chromane (600 mg, 2.41 mmol) and tributyl(1-ethoxyvinyl)stannane (1.31 g, 3.61 mmol, 1.22 mL) in dioxane (10 mL) was added TEA (609.45 mg, 6.02 mmol, 838.31 μL) and Pd(PPh₃)₂Cl₂ (169.10 mg, 240.91 μmol). The mixture was purged with Ar and stirred at 100° C. for 10 h. After cooling to rt 2 M HCl was added dropwise to pH ˜ 2, and the mixture was stirred for 3 h. The mixture was then filtered and the filtrate was extracted with EtOAc. The combined organic layer was poured into aq. KF (30 mL, about 10 g KF) and stirred for 20 min. The mixture was filtered and the organic layer was washed with brine and dried over Na₂SO₄. The solvent was removed under reduced pressure and the crude residue was purified by column chromatography (SiO₂, Petroleum ether: Ethyl acetate=1:0 to 5:1) to give 1-(4,4-difluorochroman-8-yl)ethanone (460 mg, 89.98% yield). ¹H NMR (400 MHz, METHANOL-d₄) δ ppm 7.81-7.75 (m, 2H) 7.11 (t, J=7.6 Hz, 1H) 4.52-4.46 (m, 2H) 2.59 (s, 3H) 2.58-2.50 (m, 2H).

### Step 3.

A mixture of 1-(4,4-difluorochroman-8-yl)ethanone (460 mg, 2.17 mmol) and (R)-2-methylpropane-2-sulfinamide (525.49 mg, 4.34 mmol) in THF (5 mL) was added Ti(OEt)₄ (1.98 g, 8.67 mmol, 1.80 mL) at 25° C. The mixture was stirred at 80° C. for 10 h. After cooling to 0° C. MeOH (69.46 mg, 2.17 mmol, 87.73 μL) was added, followed by LiBH₄ (51.95 mg, 2.38 mmol) and the mixture was stirred at 0° C. for 1 h. Water was added and the mixture was filtered. The filtrate was extracted with EtOAc and the combined organic layers were washed with brine and dried over Na₂SO₄. The solvent was removed under reduced pressure and the residue was purified by column chromatography to give N-[1-(4,4-difluorochroman-8-yl)ethyl]-2-methyl-propane-2-sulfinamide (480 mg, 69.76% yield). ¹H NMR (400 MHz, METHANOL-d₄) δ ppm 7.49 (t, J=9.2 Hz, 2H) 7.03 (t, J=7.6 Hz, 1H) 4.77 (q, J=6.7 Hz, 1H) 4.41 (t, J=5.2 Hz, 2H) 2.43-2.56 (m, 2H) 1.46 (d, J=6.8 Hz, 3H) 1.21 (s, 9H).

### Step 4.

A solution of N-[1-(4,4-difluorochroman-8-yl)ethyl]-2-methyl-propane-2-sulfinamide (200.00 mg, 630.13 μmol) in HCl/MeOH (4 M, 5 mL) was stirred at 25° C. for 1 h. The reaction mixture was concentrated under reduced pressure to give 1-(4,4-difluorochroman-8-yl)ethanamine hydrochloride (150 mg, 95.34% yield), which was used without further purification. LCMS (ESI): m/z: [M+H] calculated for C₁₁H₁₄F₂NO: 214.1. found 214.2.

### Step 5.

To a solution of 1-(4,4-difluorochroman-8-yl)ethanamine hydrochloride (150 mg, 600.76 μmol) and (2,4-dichloro-5,7-dihydropyrrolo[3,4-d]pyrimidin-6-yl)-morpholino-methanone (182.12 mg, 600.76 μmol) in n-BuOH (1.5 mL) was added DIEA (388.21 mg, 3.00 mmol, 523.19 μL). The mixture was stirred at 80° C. for 10 h. After cooling to rt the solvent as removed under reduced pressure and the crude residue was purified by prep-HPLC to give [2-chloro-4-[1-(4,4-difluorochroman-8-yl)ethylamino]-5,7-dihydropyrrolo[3,4-d]pyrimidin-6-yl]-morpholino-methanone (99.1 mg, 34.18% yield) as a white solid. LCMS (ESI): m/z: [M+H] calculated for C₂₂H₂₅ClF₂N5O₃: 480.2. found 480.2; ¹H NMR (400 MHz, METHANOL-d₄) δ ppm 7.46 (d, J=7.6 Hz, 1H) 7.39 (d, J=7.6 Hz, 1H) 6.99 (t, J=7.6 Hz, 1H) 5.58 (d, J=6.0 Hz, 1H) 4.63 (s, 2H) 4.55 (d, J=2.4 Hz, 2H) 4.43 (t, J=5.6 Hz, 2H) 3.72 (t, J=4.6 Hz, 4H) 3.35 (t, J=4.6 Hz, 4H) 2.56-2.46 (m, 2H) 1.52 (d, J=6.8 Hz, 3H).

### Example 477. Synthesis of 2-chloro-N-[(1R)-1-[2-methyl-3-(trifluoromethyl)phenyl]ethyl]-6-(morpholine-4-carbonyl)-5H,6H,7H-pyrrolo[3,4-d]pyrimidin-4-amine

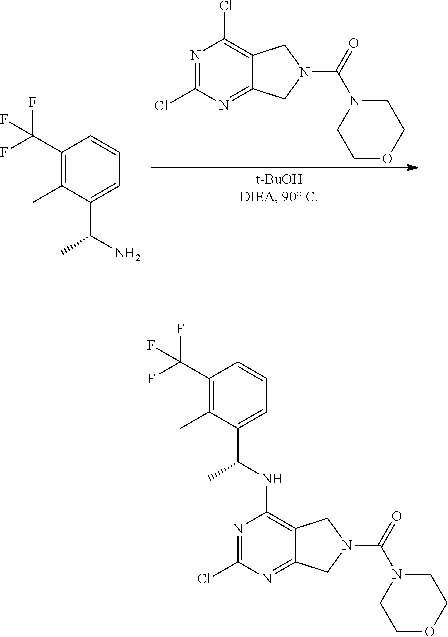 [SMILES: Cc1c([C@@H](C)N)cccc1C(F)(F)F ; O=C(N1CCOCC1)N1Cc2nc(Cl)nc(Cl)c2C1 ; Cc1c([C@@H](C)Nc2nc(Cl)nc3c2CN(C(=O)N2CCOCC2)C3)cccc1C(F)(F)F]

### Step 1.

A mixture of (1R)-1-[2-methyl-3-(trifluoromethyl)phenyl]ethanamine (70 mg, 344.48 μmol), (2,4-dichloro-5,7-dihydropyrrolo[3,4-d]pyrimidin-6-yl)-morpholino-methanone (114.87 mg, 378.93 μmol) and DIEA (222.61 mg, 1.72 mmol, 300.01 μL) in t-BuOH (2.1 mL) was sparged with N₂ and stirred at 90° C. for 15 h under N₂. After cooling to rt the solvent was removed under reduced pressure and the crude residue was purified by prep-HPLC to give [2-chloro-4-[[(1R)-1-[2-methyl-3-(trifluoromethyl)phenyl]ethyl]amino]-5,7-dihydropyrrol[3,4-d]pyrimidin-6-yl]-morpholino-methanone (29 mg, 17.72% yield). LCMS (ESI): m/z: [M+H] calculated for C₂₁H₂₄ClF₃N₅O₂: 470.15. found 470.1; ¹H NMR (400 MHz, METHANOL-d₄) δ=7.62 (d, J=7.7 Hz, 1H), 7.54 (d, J=7.8 Hz, 1H), 7.36-7.27 (m, 1H), 5.60 (q, J=7.0 Hz, 1H), 4.63 (s, 2H), 4.56 (d, J=3.9 Hz, 2H), 3.75-3.70 (m, 4H), 3.35-3.34 (m, 4H), 2.60 (s, 3H), 1.54 (d, J=7.1 Hz, 3H)

### Example 478. Synthesis of 2-chloro-N-[(1R)-1-[3-(1,1-difluoroethyl)-2-fluorophenyl]ethyl]-6-(morpholine-4-carbonyl)-5H,6H,7H-pyrrolo[3,4-d]pyrimidin-4-amine

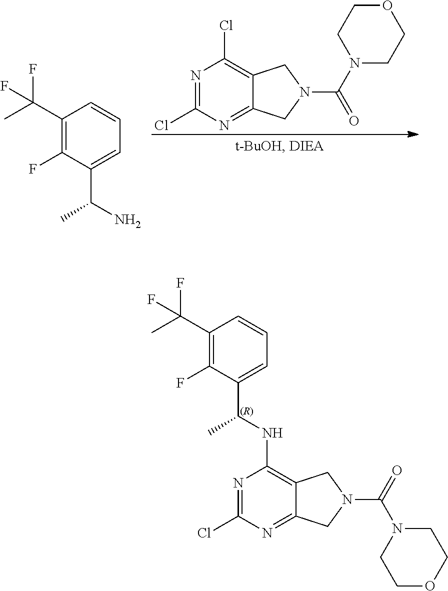

### Step 1.

To a solution of (1R)-1-[3-(1,1-difluoroethyl)-2-fluoro-phenyl]ethanamine (90 mg, 442.90 μmol) in t-BuOH (2 mL) were added (2,4-dichloro-5,7-dihydropyrrolo[3,4-d]pyrimidin-6-yl)-morpholino-methanone (134.26 mg, 442.90 μmol) and DIEA (171.73 mg, 1.33 mmol, 231.44 μL). Then the mixture was stirred at 90° C. for 2 h. After cooling to rt the mixture was filtered and the filter cake was purified by prep-HPLC to give [2-chloro-4-[[(1R)-1-[3-(1,1-difluoroethyl)-2-fluoro-phenyl]ethyl]amino]-5,7-dihydropyrrolo[3,4-d]pyrimidin-6-yl]-morpholino-methanone (70.92 mg, 149.03 μmol, 33.65% yield, 98.74% purity). LCMS (ESI): m/z: [M+H] calculated for C₂₁H₂₄ClF₃N₅O₂: 470.2. found 470.2; ¹H NMR (400 MHz, METHANOL-d₄) δ ppm 7.50 (t, J=7.2 Hz, 1H), 7.45 (t, J=7.2 Hz, 1H), 7.16-7.24 (m, 1H), 5.59 (m, 1H), 4.65 (s, 2H), 4.57 (d, J=2.4 Hz, 2H), 3.69-3.76 (m, 4H), 3.34-3.38 (m, 4H), 1.99 (t, J=18.6 Hz, 3H), 1.59 (d, J=7.2 Hz, 3H).

### Example 479. Synthesis of 2-chloro-N-[(1R)-1-{3-[1,1-difluoro-2-(oxetan-3-yloxy)ethyl]phenyl}ethyl]-6-(morpholine-4-carbonyl)-5H,6H,7H-pyrrolo[3,4-d]pyrimidin-4-amine

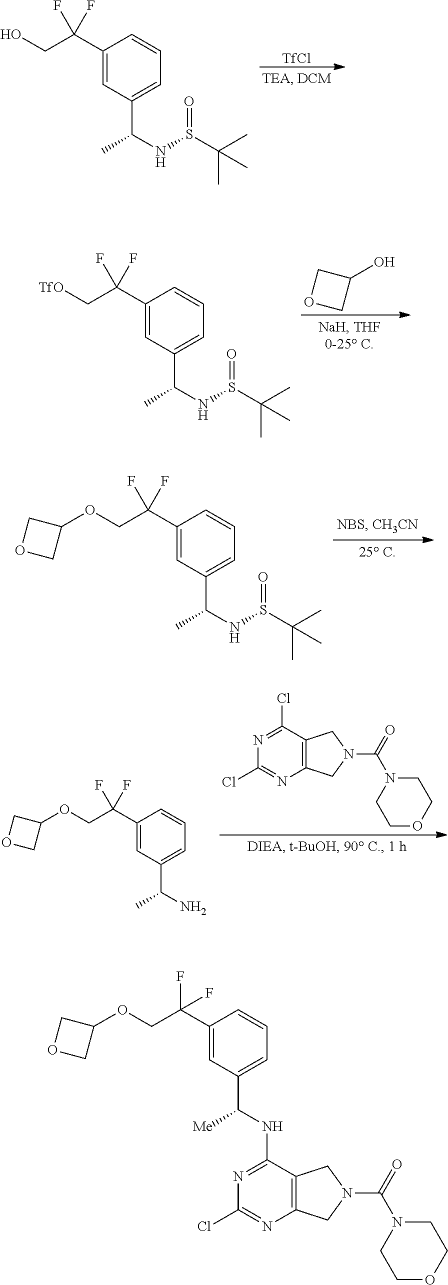

### Step 1.

To a solution of N-[(1R)-1-[3-(1,1-difluoro-2-hydroxy-ethyl) phenyl]ethyl]-2-methyl-propane-2-sulfinamide (12.08 g, 39.56 mmol) in DCM (120 mL) was added trifluoromethanesulfonyl chloride (10 g, 59.34 mmol, 6.29 mL) and Et₃N (16.01 g, 158.24 mmol, 22.02 mL) at −60° C. The cooling bath was removed and the mixture was stirred at 25° C. for 2 h. Aqueous ammonium chloride was added and the mixture was extracted with EtOAc. The combined organic phase was washed with brine and dried over Na₂SO₄. The solvent was removed under reduced pressure and the crude residue was purified by column chromatography to give [2-[3-[(1R)-1-[[(R)-tert-butylsulfinyl]amino]ethyl]phenyl]-2,2-difluoro-ethyl]trifluoromethanesulfonate (10 g, 57.79% yield). LCMS (ESI): m/z: [M+H] calculated for C₁₅H₂₁F₅NO₄S₂: 438.1. found 438.0.

### Step 2.

To a solution of oxetan-3-ol (677.37 mg, 9.14 mmol) in THF (5 mL) was added NaH (109.72 mg, 2.74 mmol, 60% wt in mineral oil) at 0° C. The mixture was stirred at 0° C. for 1 h before [2-[3-[(1R)-1-[[(R)-tert-butylsulfinyl]amino]ethyl]phenyl]-2,2-difluoro-ethyl] trifluoromethanesulfonate (0.4 g, 914.40 μmol) was added and the mixture was stirred at 25° C. for 10 h. The reaction mixture was poured into ice water and extracted with EtOAc. The combined organic phase was washed with brine and dried over Na₂SO₄. The solvent was removed under reduced pressure and the crude residue was purified by prep-HPLC to give N-[(1R)-1-[3-[1,1-difluoro-2-(oxetan-3-yloxy)ethyl]phenyl]ethyl]-2-methyl-propane-2-sulfinamide (100 mg, 30.26% yield). LCMS (ESI): m/z: [M+H] calculated for C₁₇H₂₆F₂NO₃S: 362.2. found 362.1.

### Step 3.

To a solution of N-[(1R)-1-[3-[1,1-difluoro-2-(oxetan-3-yloxy) ethyl]phenyl] ethyl]-2-methyl-propane-2-sulfinamide (70 mg, 193.67 μmol, 1 eq) in MeCN (3 mL) was added NBS (34.47 mg, 193.67 μmol) and the reaction was stirred at 25° C. for 1 h. The mixture was poured into aq.Na₂SO₃, stirred for 15 min and filtered. The solvent was removed under reduced pressure to give (1R)-1-[3-[1,1-difluoro-2-(oxetan-3-yloxy)ethyl]phenyl]ethanamine (49 mg, crude), which was used without further purification. LCMS (ESI): m/z: [M+H] calculated for C₁₃H₁₈F₂NO₂: 258.1. found 258.1.

### Step 4.

To a solution of (1R)-1-[3-[1,1-difluoro-2-(oxetan-3-yloxy)ethyl]phenyl]ethanamine (49 mg, 190.46 μmol) and (2,4-dichloro-5,7-dihydropyrrolo[3,4-d]pyrimidin-6-yl)-morpholino-methanone (57.74 mg, 190.46 μmol) in t-BuOH (2 mL) was added DIEA (49.23 mg, 380.91 μmol, 66.35 μL). The mixture was stirred at 90° C. for 1 h. After cooling to rt the mixture was filtered and the filter cake was purified by prep-HPLC to give [2-chloro-4-[[(1R)-1-[3-[1,1-difluoro-2-(oxetan-3-yloxy)ethyl]phenyl]ethyl]amino]-5,7-dihydropyrrolo[3,4-d]pyrimidin-6-yl]-morpholino-methanone (28 mg, 27.15% yield). LCMS (ESI): m/z: [M+H] calculated for C₂₄H₂₉ClF₂N₅O₄: 524.2. found 524.2; ¹H NMR (400 MHz, METHANOL-d4) δ=7.58 (s, 1H), 7.53 (d, J=6.8 Hz, 1H), 7.39-7.46 (m, 2H), 5.37-5.45 (m, 1H), 4.67-4.88 (m, 5H), 4.62-4.66 (m, 2H), 4.46-4.50 (m, 1H), 4.35-4.45 (m, 1H), 3.88-3.93 (m, 2H), 3.70-3.73 (m, 4H), 3.33-3.36 (m, 4H), 1.58 (d, J=7.2 Hz, 3H)

### Example 480. Synthesis of 2-chloro-6-(morpholine-4-carbonyl)-N-[(1R)-1-[5-(trifluoromethyl)pyridin-3-yl]ethyl]-5H,6H,7H-pyrrolo[3,4-d]pyrimidin-4-amine

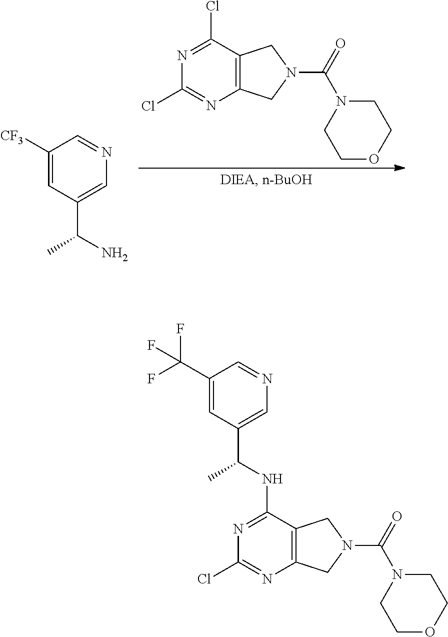 [SMILES: C[C@@H](N)c1cncc(C(F)(F)F)c1 ; O=C(N1CCOCC1)N1Cc2nc(Cl)nc(Cl)c2C1 ; C[C@@H](Nc1nc(Cl)nc2c1CN(C(=O)N1CCOCC1)C2)c1cncc(C(F)(F)F)c1]

### Step 1.

To a solution of (1R)-1-[5-(trifluoromethyl)-3-pyridyl]ethanamine hydrochloride (115 mg, 507.44 μmol) and (2,4-dichloro-5,7-dihydropyrrolo[3,4-d]pyrimidin-6-yl)-morpholino-methanone (153.83 mg, 507.44 μmol) in n-BuOH (2 mL) was added DIEA (131.17 mg, 1.01 mmol, 176.77 μL) and the mixture was stirred at 90° C. for 1h. After cooling to rt the reaction mixture was filtered and the solvent was removed under reduced pressure. The crude residue was purified by prep-HPLC to give [2-chloro-4-[[(1R)-1-[5-(trifluoromethyl)-3-pyridyl]ethyl]amino]-5,7-dihydropyrrolo[3,4-d]pyrimidin-6-yl]-morpholino-methanone (23.5 mg, 10.08% yield). LCMS (ESI): m/z: [M+H] calculated for C₁₉H₂₁ClF₃N₆O₂: 457.1. found 457.1; H NMR (400 MHz, METHANOL-d₄) δ ppm 8.87 (s, 1H) 8.77 (s, 1H) 8.17 (s, 1H) 5.44 (q, J=7.2 Hz, 1H) 4.65 (s, 2H) 4.58 (s, 2H) 3.72 (t, J=4.6 Hz, 4H) 3.36 (t, J=4.6 Hz, 4H) 1.65 (d, J=6.8 Hz, 3H).

### Example 481. Synthesis of 2-chloro-N-[(1R)-1-[5-(difluoromethyl)-4-fluorothiophen-3-yl]ethyl]-6-(morpholine-4-carbonyl)-5H,6H,7H-pyrrolo[3,4-d]pyrimidin-4-amine

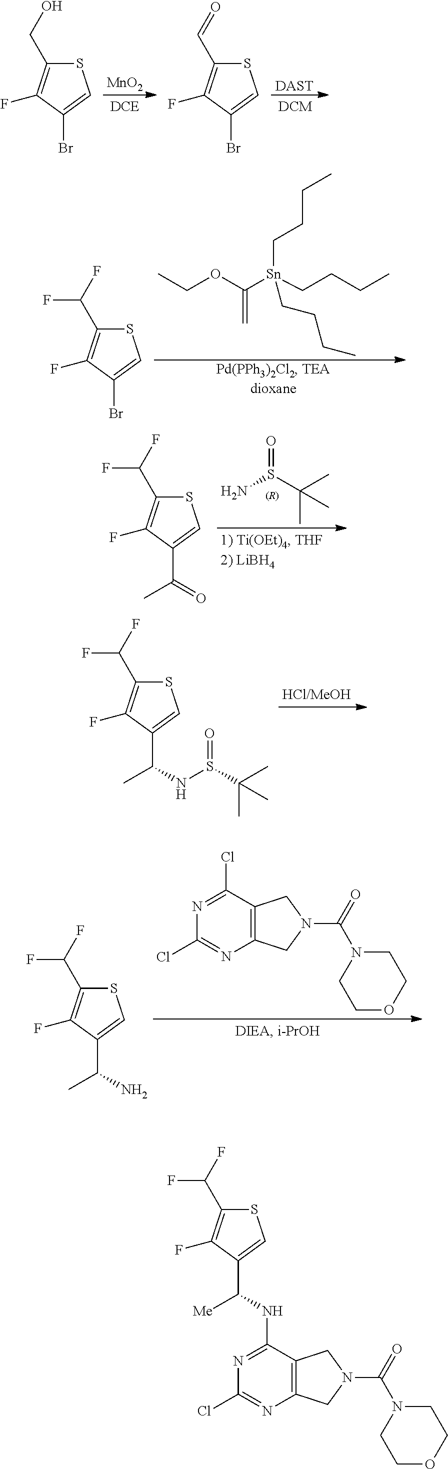

### Step 1.

To a solution of (4-bromo-3-fluoro-2-thienyl) methanol (1.6 g, 7.58 mmol) in DCE (70 mL) was added MnO₂ (3.30 g, 37.91 mmol). Then the mixture was refluxed for 2 h at 85° C. After cooling to rt the mixture was filtered and the solvent was removed under reduced pressure to give 4-bromo-3-fluoro-thiophene-2-carbaldehyde (1.4 g, 88.34% yield). ¹H NMR (400 MHz, CHLOROFORM-d) δ=10.04 (m, 1H), 7.63 (m, 1H).

To a mixture of 4-bromo-3-fluorothiophene-2-carbaldehyde (1.4 g, 6.70 mmol) in DCM (14 mL) was added DAST (2.70 g, 16.74 mmol, 2.21 mL) at 0° C. under N₂. The mixture was stirred at 20° C. for 2 h. The mixture was poured into water and extracted with EtOAc. The combined organic phase was washed with brine, dried over Na₂SO₄ and the solvent was removed under reduced pressure. The crude residue was purified by column chromatography to give 4-bromo-2-(difluoromethyl)-3-fluorothiophene (850 mg, 54.93% yield). ¹H NMR (400 MHz, CHLOROFORM-d) δ=7.34-7.39 (m, 1H), 6.77-7.08 (m, 1H).

### Step 3.

To a mixture of 4-bromo-2-(difluoromethyl)-3-fluoro-thiophene (850 mg, 3.68 mmol) and tributyl(1-ethoxyvinyl)stannane (1.99 g, 5.52 mmol, 1.86 mL) in dioxane (9 mL) was added TEA (930.72 mg, 9.20 mmol, 1.28 mL) and Pd(PPh₃)₂Cl₂ (258.24 mg, 367.91 μmol). The mixture was sparged with N₂ and then stirred at 90° C. for 2 h under N₂. After cooling to rt the pH was adjusted to pH=2 using aqueous HCl and the mixture was stirred for 30 min. After filtration the filtrate was extracted with EtOAc. The combined organic phase was poured into aq.KF (30 mL, about 3 g KF) and stirred for 1 h. The mixture was filtered and the organic layer was washed with brine and dried over Na₂SO₄. The solvent was removed under reduced pressure and the crude residue was purified by column chromatography to give 1-[5-(difluoromethyl)-4-fluoro-3-thienyl]ethanone (630 mg, 88.19% yield). ¹H NMR (400 MHz, CHLOROFORM-d) δ=8.12 (d, J=4.0 Hz, 1H), 6.78-7.13 (m, 1H), 2.54 (d, J=2.8 Hz, 3H).

### Step 4.

To a solution of 1-[5-(difluoromethyl)-4-fluoro-3-thienyl]ethanone (600 mg, 3.09 mmol), (R)-2-methylpropane-2-sulfinamide (561.77 mg, 4.64 mmol) in THF (6 mL) was added Ti(OEt)₄ (2.11 g, 9.27 mmol, 1.92 mL) and the mixture was stirred at 80° C. for 3 h. The reaction was then cooled to 0° C. and LiBH₄ (87.51 mg, 4.02 mmol) was added. After 30 min. the reaction was poured into ice water and the resulting mixture was filtered. The filtrate was extracted with EtOAc and the combined organic phases were dried over Na₂SO₄ The solvent was removed under reduced pressure and the residue was purified by column chromatography to give N-[(1R)-1-[5-(difluoromethyl)-4-fluoro-3-thienyl]ethyl]-2-methyl-propane-2-sulfinamide (520 mg, 56.21% yield). ¹H NMR (400 MHz, CHLOROFORM-d) δ=7.32 (d, J=4.6, 1H), 6.77-7.05 (m, 1H), 4.58 (m, 1H), 3.45 (d, J=4.4, 1H), 1.56 (d, J=6.8, 3H), 1.23 (s, 9H).

### Step 5.

To a mixture of N-[(1R)-1-[5-(difluoromethyl)-4-fluoro-3-thienyl]ethyl]-2-methyl-propane-2-sulfinamide (200 mg, 668.06 μmol) in MeOH (2 mL) was added HCl/MeOH (4 M, 668.06 μL) at 25° C. The mixture was stirred at 25° C. for 2 h. The solvent was then removed under reduced pressure and the residue was dissolve in MeOH (2 mL). MeOH/NaOH was added to adjust the pH of the mixture to 8. The resulting residue was triturate with DCM/MeOH=10/1, filtered and the solvent was removed under reduced pressure to give (1R)-1-[5-(difluoromethyl)-4-fluoro-3-thienyl]ethanamine (120 mg, 92.02% yield). LCMS (ESI): m/z: [M+H] calculated for C₇H₉F₃NS: 196.1 found 196.1.

### Step 6.

To a solution of (1R)-1-[5-(difluoromethyl)-4-fluoro-3-thienyl]ethanamine (120 mg, 614.74 μmol) in t-BuOH (3 mL) were added (2,4-dichloro-5,7-dihydropyrrolo[3,4-d]pyrimidin-6-yl)-morpholino-methanone (186.35 mg, 614.74 μmol) and DIEA (238.35 mg, 1.84 mmol, 321.23 μL). The mixture was stirred at 90° C. for 2 h. After cooling to rt the residue was purified by prep-HPLC to give [2-chloro-4-[[(1R)-1-[5-(difluoromethyl)-4-fluoro-3-thienyl]ethyl]amino]-5,7-dihydropyrrolo[3,4-d]pyrimidin-6-yl]-morpholino-methanone (58.6 mg, 20.62% yield). LCMS (ESI): m/z: [M+H] calculated for C₁₈H₂₀ClF₃N₅O₂S: 462.1 found 462.0; ¹H NMR (400 MHz, METHANOL-d₄) δ=7.50 (d, J=4.4, 1H), 6.86-7.23 (m, 1H), 5.44 (m, 1H), 4.57-4.63 (m, 4H), 3.70-3.75 (m, 4H), 3.34-3.37 (m, 4H), 1.59 (d, J=7.2, 3H).

### Example 482. Synthesis of 2-{3-[(1R)-1-{[2-chloro-6-(morpholine-4-carbonyl)-5H,6H,7H-pyrrolo[3,4-d]pyrimidin-4-yl]amino}ethyl]phenyl}-2-methylpropanenitrile

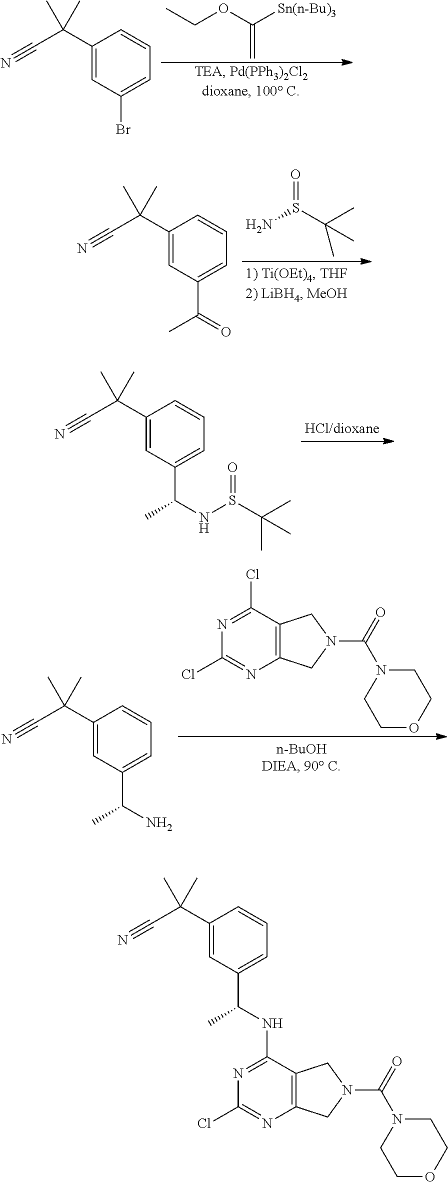

### Step 1.

To a mixture of 2-(3-bromophenyl)-2-methyl-propanenitrile (2 g, 8.92 mmol) in dioxane (20 mL) was added TEA (2.26 g, 22.31 mmol, 3.11 mL) and tributyl (1-ethoxyvinyl) stannane (3.87 g, 10.71 mmol, 3.61 mL) and Pd(PPh₃)₂Cl₂ (626.42 mg, 892.47 μmol). The mixture was stirred at 100° C. under Argon for 3 h. After cooling to rt the reaction mixture was filtrated and 1M HCl (20 mL) was added to the filtrate. The mixture was stirred at 20° C. for 0.5 h, aqueous KF was added and the reaction was stirred for 0.5 h. After extraction with EtOAc the combined organic phases were dried over Na₂SO₄ and the solvent was removed under reduced pressure. The crude residue was purified by column chromatography to give 2-(3-acetylphenyl)-2-methyl-propanenitrile (1.4 g, 83.78% yield). ¹H NMR (400 MHz, CHLOROFORM-d) δ ppm 8.06 (d, J=1.6 Hz, 1H) 7.91 (d, J=8 Hz, 1H) 7.70-7.77 (m, 1H) 7.48-7.56 (m, 1H) 2.64 (s, 3H) 1.74-1.82 (s, 6H).

### Step 2.

To a solution of 2-(3-acetylphenyl)-2-methyl-propanenitrile (900 mg, 4.81 mmol) and (R)-2-methylpropane-2-sulfinamide (640.84 mg, 5.29 mmol) in THF (15 mL) was added Ti(OEt)₄ (2.19 g, 9.61 mmol, 1.99 mL). The mixture was stirred at 70° C. for 16 h. After cooling to rt the mixture was poured into water and extracted with EtOAc. The combined organic layers were washed with brine and dried over Na₂SO₄. The solvent was removed under reduced pressure to give (NZ)-N-[1-[3-(1-cyano-1-methyl-ethyl)phenyl]ethylidene]-2-methyl-propane-2-sulfinamide(1.40 g, crude) as ayellow oil. To a mixture of (NZ)-N-[1-[3-(1-cyano-1-methyl-ethyl)phenyl]ethylidene]-2-methyl-propane-2-sulfinamide (1.4 g, 4.82 mmol) in MeOH (15 mL) was added LiBH₄ (210.02 mg, 9.64 mmol) at −30° C. under N₂. The cooling bath was removed and the reaction was stirred at rt for 30 min. before H₂O (20 mL) was added and the aqueous phase was extracted with EtOAc. The combined organic phase was washed with brine and dried over Na₂SO₄. The solvent was removed under reduced pressure and the crude residue was purified by column chromatography to give N-[(1R)-1-[3-(1-cyano-1-methyl-ethyl)phenyl]ethyl]-2-methyl-propane-2-sulfinamide (550 mg, 39.02% yield). ¹H NMR (400 MHz, CHLOROFORM-d) δ ppm 7.40 (s, 1H) 7.26-7.34 (m, 2H) 7.18-7.25 (m, 1H) 4.54 (qd, J=6.62, 3.00 Hz, 1H) 3.27 (br d, J=2.08 Hz, 1H) 1.66 (d, J=0.98 Hz, 6H) 1.47 (d, J=6.60 Hz, 3H) 1.07-1.22 (m, 9H).

### Step 3.

The mixture of N-[(1R)-1-[3-(1-cyano-1-methyl-ethyl)phenyl]ethyl]-2-methyl-propane-2-sulfinamide (170 mg, 581.32 μmol) in HCl/dioxane (4 M, 1.45 mL) was stirred for 1 h at 20° C. The solvent was removed under reduced pressure to give 2-[3-[(1R)-1-aminoethyl]phenyl]-2-methyl-propanenitrile hydrochloride (130 mg, 99.51% yield), which was used without further purification. H NMR (400 MHz, CHLOROFORM-d) δ ppm 8.66 (br s, 3H) 7.63 (s, 1H) 7.49 (br d, J=7.03 Hz, 1H) 7.34-7.46 (m, 2H) 4.45 (s, 1H) 3.71 (s, 3H) 1.72 (d, J=10.85 Hz, 6H) 1.42 (s, 3H).

### Step 4.

To a mixture of 2-[3-[(1R)-1-aminoethyl]phenyl]-2-methyl-propanenitrile hydrochloride (130 mg, 578.47 μmol) and (2,4-dichloro-5,7-dihydropyrrolo[3,4-d]pyrimidin-6-yl)-morpholino-methanone (157.82 mg, 520.63 μmol) in t-BuOH (2 mL) was added DIEA (373.82 mg, 2.89 mmol, 503.80 μL). The mixture was stirred at 90° C. under N₂ for 1 h. The solvent was removed under reduced pressure and the crude residue was purified by prep-HPLC to give 2-[3-[(1R)-1-[[2-chloro-6-(morpholine-4-carbonyl)-5,7-dihydropyrrolo[3,4-d]pyrimidin-4-yl]amino]ethyl]phenyl]-2-methyl-propanenitrile (67.31 mg, 23.68% yield). LCMS (ESI): m/z: [M+H] calculated for C₂₃H₂₈ClN₆O₂: 455.2. found 455.2; ¹H NMR (400 MHz, METHANOL-d₄) δ ppm 7.59 (s, 1H) 7.39 (s, 3H) 5.43 (d, J=5.07 Hz, 1H) 4.63 (s, 4H) 3.72 (s, 4H) 3.36 (s, 4H) 1.73 (d, J=1.54 Hz, 6H) 1.62 (d, J=4.85 Hz, 3H)

### Example 483. Synthesis of 2-chloro-N-[(1R)-1-{3-[2-(dimethylamino)-1,1-difluoroethyl]phenyl}ethyl]-6-(morpholine-4-carbonyl)-5H,6H,7H-pyrrolo[3,4-d]pyrimidin-4-amine

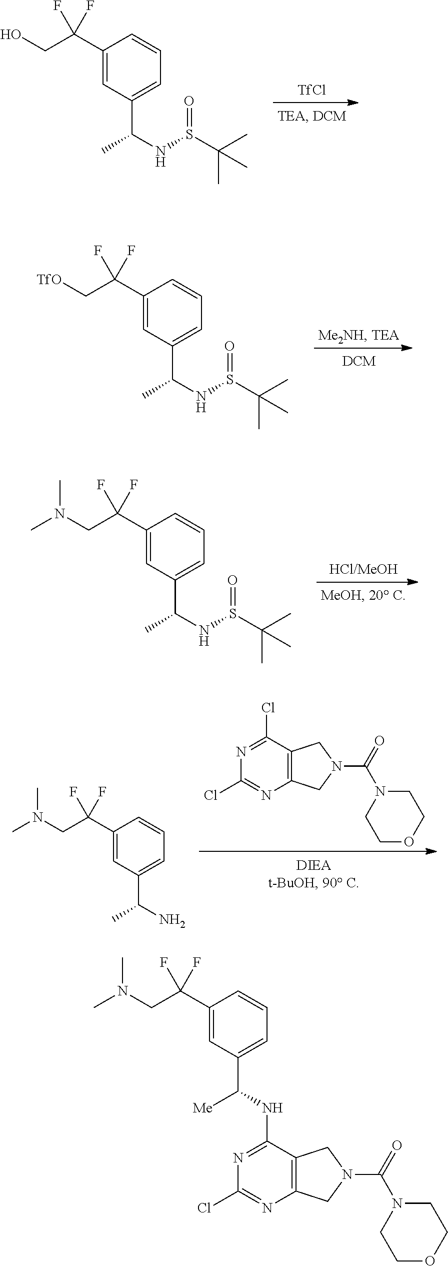

### Step 1.

To a solution of N-[(1R)-1-[3-(1, 1-difluoro-2-hydroxy-ethyl) phenyl]ethyl]-2-methyl-propane-2-sulfinamide (2.00 g, 6.55 mmol) in DCM (20 mL) was added trifluoromethanesulfonyl chloride (1.66 g, 9.82 mmol, 1.04 mL) and Et₃N (2.65 g, 26.20 mmol, 3.65 mL) at −78° C. The cooling bath was removed and the mixture was stirred at 25° C. for 2 h. The reaction mixture was quenched by the addition of aqueous ammonium chloride, diluted with water and extracted with EtOAc. The organic phase was washed with brine and dried over Na₂SO₄. The solvent was removed under reduced pressure to give [2-[3-[(1R)-1-[[(R)-tert-butylsulfinyl] amino]ethyl]phenyl]-2,2-difluoro-ethyl]trifluoromethanesulfonate (2 g, 69.81% yield), which was used without further purification. LCMS (ESI): m/z: [M+H] calculated for C₁₋₆H₂₁F₅NO₄S₂: 438.2. found 438.0.

### Step 2.

To a solution of [2-[3-[(1R)-1-[[(R)-tert-butylsulfinyl]amino]ethyl]phenyl]-2,2-difluoro-ethyl] trifluoromethanesulfonate (2.00 g, 4.57 mmol) in DCM (20 mL) was added Me₂NH (4 M, 3.43 mL) and TEA (1.85 g, 18.29 mmol, 2.55 mL) at 0° C. The cooling bath was removed and the mixture was stirred at 25° C. for 2 h. The reaction was quenched by the addition of aqueous ammonium chloride, diluted with water and extracted with EtOAc. The organic phase was washed with brine and dried over Na₂SO₄. The residue was purified by column chromatography to give N-[(1R)-1-[3-[2-(dimethylamino)-1,1-difluoro-ethyl]phenyl]ethyl]-2-methyl-propane-2-sulfinamide (1.3 g, 76.98% yield). LCMS (ESI): m/z: [M+H] calculated for C₁₋₆H₂₇F₂N₂OS: 333.2. found 333.1.

### Step 3.

To a solution of N-[(1R)-1-[3-[2-(dimethylamino)-1,1-difluoro-ethyl] phenyl]ethyl]-2-methyl-propane-2-sulfinamide (500.00 mg, 1.50 mmol) in MeOH (5 mL) was added HCl/MeOH (4 M, 1.50 mL) and the mixture was stirred at 20° C. for 2 h. The solvent was removed under reduced pressure and the residue was triturated with MTBE and acetonitrile. The solid was dissolve in MeOH (2 mL) and MeOH/NaOH solution was added until pH=8. After filtration the solvent was removed under reduced pressure and the residue was triturated with DCM: MeOH=10:1, followed by acetonitrile. After filtration the filter cake was dried under vacuum to give 2-[3-[(1R)-1-aminoethyl]phenyl]-2,2-difluoro-N,N-dimethyl-ethanamine (0.3 g, 87.38% yield), which was used without further purification. LCMS (ESI): m/z: [M+H] calculated for C12H₁₉F₂N₂: 229.1. found 229.4.

### Step 4.

To a solution of 2-[3-[(1R)-1-aminoethyl]phenyl]-2,2-difluoro-N,N-dimethyl-ethanamine (100 mg, 438.06 μmol) in t-BuOH (2 mL) was added DIEA (169.85 mg, 1.31 mmol, 228.90 μL) and (2,4-dichloro-5,7-dihydropyrrolo[3,4-d]pyrimidin-6-yl)-morpholino-methanone (132.79 mg, 438.06 μmol). The reaction was stirred at 90° C. for 3 h. After cooling to rt the mixture was filtered and the filter cake was purified by prep-HPLC to give [2-chloro-4-[[(1R)-1-[3-[2-(dimethylamino)-1,1-difluoro-ethyl]phenyl]ethyl]amino]-5,7-dihy dropyrrolo[3,4-d]pyrimidin-6-yl]-morpholino-methanone (35 mg, 15.82% yield). LCMS (ESI): m/z: [M+H] calculated for C₂₄H₃₀ClF₂N₆O₂: 495.2. found 495.1 ¹H NMR (400 MHz, METHANOL-d₄) δ=7.57 (s, 1H), 7.51 (d, J=7.2 Hz, 1H), 7.45-7.37 (m, 2H), 5.40 (d, J=6.8 Hz, 1H), 4.55-4.64 (m, 4H), 3.70-3.74 (m, 4H), 3.34-3.37 (m, 4H), 2.98 (t, J=15.2 Hz, 2H), 2.27 (s, 6H), 1.59 (d, J=7.2 Hz, 3H).

### Example 484. Synthesis of 2-chloro-N-[(1R)-1-{3-[difluoro(morpholin-2-yl)methyl]phenyl}ethyl]-6-(morpholine-4-carbonyl)-5H,6H,7H-pyrrolo[3,4-d]pyrimidin-4-amine

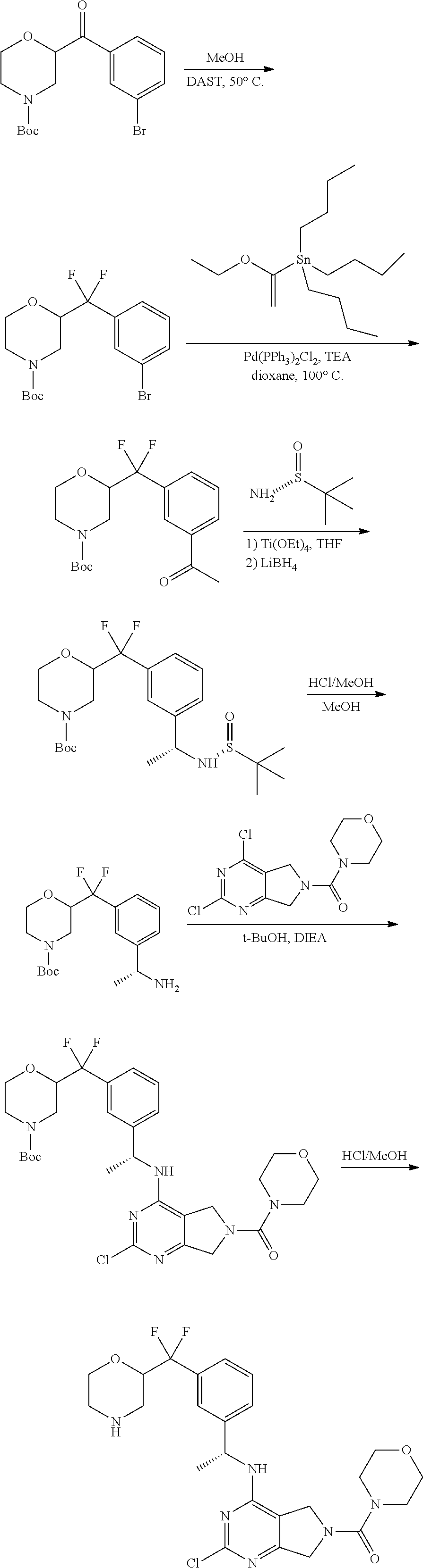

### Step 1.

To a solution of tert-butyl 2-(3-bromobenzoyl)morpholine-4-carboxylate (1.8 g, 4.86 mmol) in DAST (5 mL) was added MeOH (15.58 mg, 486.17 μmol, 19.67 μL) at 25° C. The mixture was stirred at 60° C. for 2h, cooled to rt and added into ice water. Aqueous NaHCO₃ was added until pH=7 and the mixture was extracted EtOAc. The combined organic phases were washed with brine, dried over Na₂SO₄ and the solvent was removed under reduced pressure. The crude residue was purified by column chromatography to give tert-butyl 2-[(3-bromophenyl)-difluoro-methyl]morpholine-4-carboxylate (1.1 g, 57.68% yield). ¹H NMR (CHLOROFORM-d, 400 MHz) δ ppm=7.66 (s, 1H), 7.60 (d, J=8.4 Hz, 1H), 7.44 (d, J=7.2 Hz, 1H), 7.29-7.34 (m, 1H), 4.13 (m, 1H), 3.92 (dd, J=7.4, 1H), 3.72-3.87 (m, 2H), 3.45-3.52 (m, 1H), 2.92 (s, 2H), 1.47 (s, 9H).

### Step 2.

To a mixture of tert-butyl 2-[(3-bromophenyl)-difluoro-methyl]morpholine-4-carboxylate (1.1 g, 2.80 mmol) and tributyl(1-ethoxyvinyl)stannane (1.52 g, 4.21 mmol, 1.42 mL) in dioxane (10 mL) was added TEA (709.45 mg, 7.01 mmol, 975.86 μL) and Pd(PPh₃)₂Cl, (196.84 mg, 280.44 μmol). The mixture was purged with N₂ and then stirred at 100° C. for 3 h under N₂. After cooling to rt aqueous HCl (15 mL, 0.8 M) was added to the mixture and stirred for 0.5 h (pH=2). The mixture was then filtered and the filtrate was extracted with EtOAc. The combined organic layers were poured into aq.KF (30 mL, about 3 g KF) and stirred for 1 h at rt. The mixture was filtered and the organic layer was washed with brine, dried over Na₂SO₄ and the solvent was removed under reduced pressure. The crude residue was purified by column chromatography to give tert-butyl 2-[(3-acetylphenyl)-difluoro-methyl]morpholine-4-carboxylate (835 mg, 83.78% yield). LCMS (ESI): m/z: [M+H] calculated for C₁₈H₂₄F₂NO₄: 356.2. found 356.4.

### Step 3.

To a solution of tert-butyl 2-[(3-acetylphenyl)-difluoro-methyl]morpholine-4-carboxylate (835 mg, 2.35 mmol) and (R)-2-methylpropane-2-sulfinamide (711.94 mg, 5.87 mmol) in THF (9 mL) was added Ti(OEt)₄ (2.68 g, 11.75 mmol, 2.44 mL) and the mixture was stirred at 80° C. for 3 h. After cooling to 0° C. LiBH₄ (66.54 mg, 3.05 mmol) was added and the reaction was stirred for 30 min. The mixture was then added into ice water and filtered. The filtrate was extracted with EtOAc and dried over Na₂SO₄. The solvent was removed under reduced pressure and the crude residue was purified by column chromatography to give tert-butyl2-[[3-[(1R)-1-[[(R)-tert-butylsulfinyl]amino]ethyl]phenyl]-difluoro-methyl]morpholine-4-carboxylate (570 mg, 52.67% yield). LCMS (ESI): m/z: [M+H] calculated for C₂₂H₃₅F₂N₂O₄S: 461.2. found 461.1;

### Step 4.

To a solution of tert-butyl 2-[[3-[(1R)-1-[[(R)-tert-butylsulfinyl]amino]ethyl]phenyl]-difluoro-methyl]morpholine-4-carboxylate (570 mg, 1.24 mmol) in MeOH (6 mL) was added HCl/MeOH (4 M, 618.79 μL) at 25° C. and the resulting mixture was stirred for 4 h at rt. The solvent was removed under reduced pressure and the crude residue was dissolved in MeOH (5 mL). MeOH/NaOH solution until was then added until pH=8. The mixture was filtered and the solvent was removed under reduced pressure. The crude residue was triturated with DCM: MeOH=10/1 and the solvent was removed under reduced pressure to give tert-butyl 2-[[3-[(1R)-1-aminoethyl]phenyl]-difluoro-methyl]morpholine-4-carboxylate (520 mg, crude), which was used without further purification. LCMS (ESI): m/z: [M+H] calculated for C₁₈H₂₇F₂N₂O₃: 357.2. found 357.1.

### Step 5.

To a solution of tert-butyl 2-[[3-[(1R)-1-aminoethyl]phenyl]-difluoro-methyl]morpholine-4-carboxylate (470 mg, 1.32 mmol) in t-BuOH (5 mL) were added (2,4-dichloro-5,7-dihydropyrrolo[3,4-d]pyrimidin-6-yl)-morpholino-methanone (399.76 mg, 1.32 mmol) and DIEA (511.31 mg, 3.96 mmol, 689.09 μL). The mixture was stirred at 90° C. for 2 h. After cooling to rt the reaction was quenched by the addition of 15 mL of water. The mixture was extracted with EtOAc and the combined organic layers were washed with brine and dried over Na₂SO₄ The solvent was removed under reduced pressure and the crude residue was purified by column chromatography to give tert-butyl 2-[[3-[(1R)-1-[[2-chloro-6-(morpholine-4-carbonyl)-5,7-dihydropyrrolo[3,4-d]pyrimidin-4-yl]amino]ethyl]phenyl]-difluoro-methyl]morpholine-4-carboxylate (570 mg, 65.83% yield). LCMS (ESI): m/z: [M+H] calculated for C₂₉H₃₈ClF₂N₆O₅: 623.3. found 623.1.

### Step 6.

A solution of tert-butyl 2-[[3-[(1R)-1-[[2-chloro-6-(morpholine-4-carbonyl)-5,7-dihydropyrrolo[3,4-d]pyrimidin-4-yl]amino]ethyl]phenyl]-difluoro-methyl]morpholine-4-carboxylate (470 mg, 754.31 μmol) in HCl/MeOH (5 mL) was stirred at 25° C. for 0.5 h. The solvent was removed under reduced pressure and the crude residue was dissolve in MeOH. To the resulting mixture was added MeOH/NaOH solution until pH=8. The mixture was filtered and the crud residue was triturated with DCM/MeOH=10/1. The solvent was removed under reduced pressure and the residue was purified by prep-HPLC to give [2-chloro-4-[[(1R)-1-[3-[difluoro(morpholin-2-yl)methyl]phenyl]ethyl]amino]-5,7-dihydropyrrolo[3,4-d]pyrimidin-6-yl]-morpholino-methanone (230.3 mg, 57.10% yield). LCMS (ESI): m/z: [M+H] calculated for C₂₄H₃₀ClF₂N₆O₃: 523.2. found 523.2; ¹H NMR (METHANOL-d₄, 400 MHz) δ ppm=7.52 (t, J=5.8 Hz, 2H), 7.39-7.45 (m, 1H), 7.35-7.39 (m, 1H), 5.41 (d, J=6.8 Hz, 1H), 4.62 (s, 2H), 4.57 (d, J=2.0 Hz, 2H), 3.77-3.93 (m, 2H), 3.70-3.74 (m, 4H), 3.48-3.56 (m, 1H), 3.34-3.38 (m, 4H), 2.93 (m, 1H), 2.58-2.74 (m, 3H), 1.58 (d, J=6.8 Hz, 3H).

### Example 485. Synthesis of 2-chloro-N-[(1R)-1-{3-[difluoro(4-methylmorpholin-2-yl)methyl]phenyl}ethyl]-6-(morpholine-4-carbonyl)-5H,6H,7H-pyrrolo[3,4-d]pyrimidin-4-amine

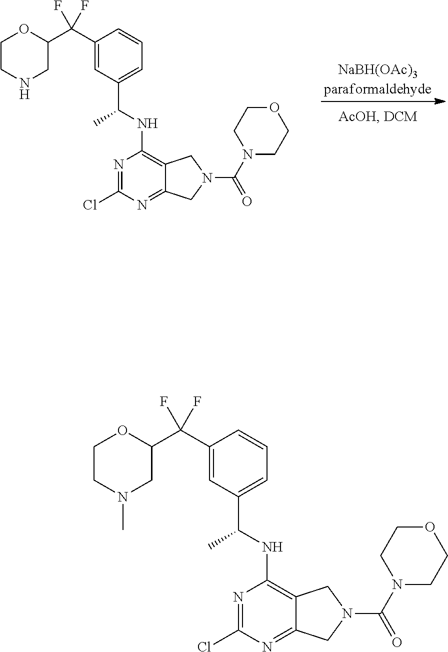 [SMILES: C[C@@H](Nc1nc(Cl)nc2c1CN(C(=O)N1CCOCC1)C2)c1cccc(C(F)(F)C2CNCCO2)c1 ; C[C@@H](Nc1nc(Cl)nc2c1CN(C(=O)N1CCOCC1)C2)c1cccc(C(F)(F)C2CN(C)CCO2)c1]

### Step 1.

To a solution of [2-chloro-4-[[(1R)-1-[3-[difluoro(morpholin-2-yl)methyl]phenyl]ethyl]amino]-5,7-dihy dropyrrolo[3,4-d]pyrimidin-6-yl]-morpholino-methanone (100 mg, 191.21 μmol) in AcOH (1.2 mL) and DCM (3 mL) was added NaBH(OAc)₃ (101.32 mg, 478.04 μmol) and paraformaldehyde (100 mg). The mixture was stirred at 25° C. for 2 h. The mixture was poured into ice water and extracted with EtOAc. The combined organic phase was washed with brine and dried over Na₂SO₄. The solvent was removed under reduced pressure and the crude residue was purified by prep-HPLC to give [2-chloro-4-[[(1R)-1-[3-[difluoro-(4-methylmorpholin-2-yl)methyl]phenyl]ethyl]amino]-5,7-dihydropyrrolo[3,4-d]pyrimidin-6-yl]-morpholino-methanone (8.3 mg, 7.68% yield). LCMS (ESI): m/z: [M+H] calculated for C₂₅H₃₂ClF₂N₆O₃: 537.2. found 537.3; ¹H NMR (METHANOL-d₄, 400 MHz): δ=7.50-7.55 (m, 2H), 7.40-7.45 (m, 1H), 7.35-7.40 (m, 1H), 5.33-5.45 (m, 1H), 4.63 (s, 2H), 4.57 (s, 2H), 3.88-3.98 (m, 1H), 3.84 (m, 1H), 3.70-3.76 (m, 4H), 3.57 (m, 1H), 3.33-3.39 (m, 4H), 2.82 (d, J=11.3 Hz, 1H), 2.61-2.69 (m, 1H), 2.26 (d, J=3.9 Hz, 3H), 1.89-2.11 (m, 2H), 1.59 (d, J=7.2 Hz, 3H).

### Example 486. Synthesis of 2-chloro-N-[(1R)-1-[3-(difluoromethyl)phenyl]ethyl]-6-(morpholine-4-carbonyl)-5H,6H,7H-pyrrolo[3,4-d]pyrimidin-4-amine

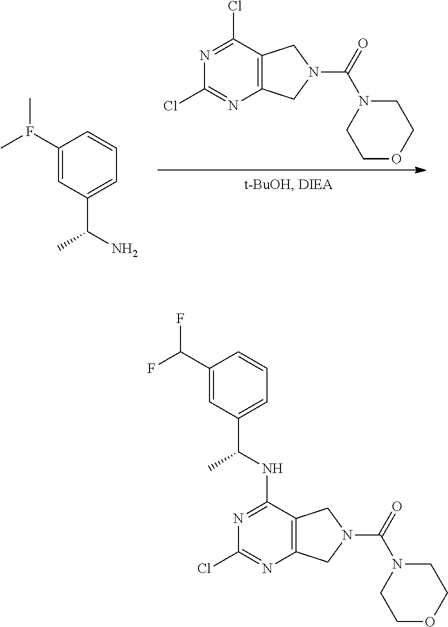

### Step 1.

To a mixture of (1R)-1-[3-(difluoromethyl)phenyl]ethanamine hydrochloride(150 mg, 722.38 μmol) and (2,4-dichloro-5,7-dihydropyrrolo[3,4-d]pyrimidin-6-yl)-morpholino-methanone (218.98 mg, 722.38 μmol) in t-BuOH (1 mL) was added DIEA (280.09 mg, 2.17 mmol, 377.48 μL). The mixture was stirred at 90° C. under N₂ for 1 h. The solvent was removed under reduced pressure and the crude residue was purified by prep-HPLC to give [2-chloro-4-[[(1R)-1-[3-(difluoromethyl)phenyl]ethyl]amino]-5,7-dihydropyrrolo[3,4-d]pyrimidin-6-yl]-morpholino-methanone (52.26 mg, 16.52% yield). LCMS (ESI): m/z: [M+H] calculated for C₂₀H₂₃ClF₂N₅O₂: 438.1. found 438.1; ¹H NMR (400 MHz, METHANOL-d₄) δ ppm 7.39-7.59 (m, 4H) 6.65 (t, J=56.8, 1H) 5.39-5.54 (m, 1H) 5.08 (d, J=6.48 Hz, 1H) 4.48-4.70 (m, 4H) 3.61-3.81 (m, 4H) 3.22-3.42 (m, 4H) 1.63 (d, J=6.97 Hz, 4H).

### Example 487. Synthesis of (R)-(2-(1,1-difluoro-2-hydroxyethyl)-4-((1-(3-(difluoromethyl)-2-fluorophenyl)ethyl)amino)-5,7-dihydro-6H-pyrrolo[3,4-d]pyrimidin-6-yl)(4-methoxytetrahydro-2H-pyran-4-yl)methanone

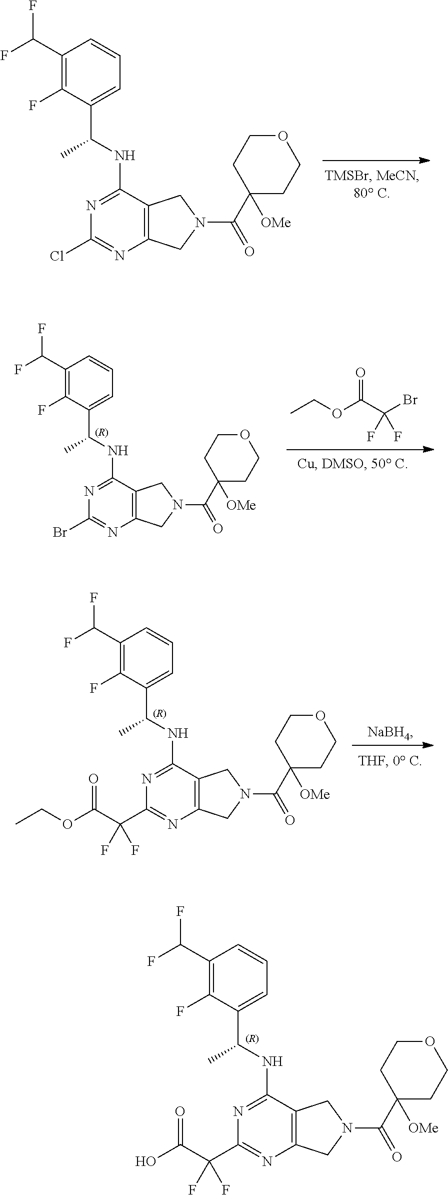

### Step 1.

To a solution of 2-chloro-N-[(1R)-1-[3-(difluoromethyl)-2-fluorophenyl]ethyl]-6-(4-methoxy-oxane-4-carbonyl)-5H,6H,7H-pyrrolo[3,4-d]pyrimidin-4-amine (1.0 g, 2.1 mmol) in anhydrous ACN (7.5 mL), trimethylbromosilane (820 μL, 6.3 mmol) was added. The reaction mixture was stirred for 3 days at 80° C. The solvent was evaporated and the residue was quenched with saturated aqueous NaHCO₃ and extracted with EtOAc. The combined organic layers were dried over Na₂SO₄, filtered and concentrated to give 2-bromo-N-[(1R)-1-[3-(difluoromethyl)-2-fluorophenyl]ethyl]-6-(4-methoxyoxane-4-carbonyl)-5H,6H,7H-pyrrolo[3,4-d]pyrimidin-4-amine (840 mg, 76% yield). LCMS (ESI) m/z: [M−H calculated for C₂₂H₂₃BrF₃N₄O₃: 527.10. found: 527.79.

### Step 2.

To a solution of ethyl bromodifluoroacetate (390 μL, 3.07 mmol) in DMSO (3.8 mL), copper (195 mg, 3.07 mmol) was added. The reaction mixture was stirred at room temperature for 1h, then 2-Bromo-N-[(1R)-1-[3-(difluoromethyl)-2-fluorophenyl]ethyl]-6-(4-methoxy-oxane-4-carbonyl)-5H,6H,7H-pyrrolo[3,4-d]pyrimidin-4-amine (650 mg, 1.23 mmol) was added and the reaction was stirred at 50° C. for 3h. The reaction mixture was quenched with ice water, filtered, and extracted with EtOAc. The combined organic layers were dried over anhydrous Na₂SO₄, filtered, concentrated under reduced pressure, and the residue purified by flash column chromatography to give ethyl 2-(4-{[(1R)-1-[3-(difluoromethyl)-2-fluorophenyl]ethyl]amino}-6-(4-methoxyoxane-4-carbonyl)-5H,6H,7H-pyrrolo-[3,4-d]pyrimidin-2-yl)-2,2-difluoroacetate (620 mg, 88% yield). LCMS (ESI): m/z: [M+H] calculated for C26H₃₀F₅N₄O₅: 573.21. found: 573.35.

### Step 3.

To a solution of 2-(4-{[(1R)-1-[3-(difluoromethyl)-2-fluorophenyl]ethyl]amino}-6-(4-methoxy-oxane-4-carbonyl)-5H,6H,7H-pyrrolo[3,4-d]pyrimidin-2-yl)-2,2-difluoroacetate (565 mg, 0.99 mmol) in anhydrous THF (28 mL) at 0° C., sodium borohydride (112 mg, 2.96 mmol) was added. The reaction mixture was stirred overnight at room temperature, then saturated NH₄Cl solution was added and the mixture extracted with EtOAc. The combined organic layers were dried over Na₂SO₄, filtered, and concentrated under reduced pressure to give 2-(4-{[(1R)-1-[3-(difluoromethyl)-2-fluorophenyl]ethyl]amino}-6-(4-methoxyoxane-4-carbonyl)-5H,6H,7H-pyrrolo[3,4-d]pyrimidin-2-yl)-2,2-difluoroethan-1-ol (477 mg; 91% yield). LCMS (ESI): m/z: [M+H] calculated for C₂₄H₂₈FSN₄O₄: 531.20. found: 531.15. ¹H NMR (300 MHz, DMSO-d₆) δ 8.28-8.22 (m, 1H), 7.63 (t, J=7.6 Hz, 1H), 7.49 (t, J=7.2 Hz, 1H), 7.37-7.24 (m, 1H), 7.42-7.00 (m, 1H), 5.61-5.49 (m, 1H), 5.35 (t, J=6.5 Hz, 1H), 4.92-4.85 (m, 2H), 4.65-4.57 (m, 2H), 4.04-3.49 (m, 6H), 3.18 (s, 3H), 2.05-1.81 (m, 4H), 1.53 (dd, J=7.1, 4.3 Hz, 3H).

### Example 488. Synthesis of 2-(2-amino-1,1-difluoroethyl)-N-[(1R)-1-[3-(difluoromethyl)-2-fluorophenyl]ethyl]-6-(4-methoxyoxane-4-carbonyl)-5H,6H,7H-pyrrolo[3,4-d]pyrimidin-4-amine

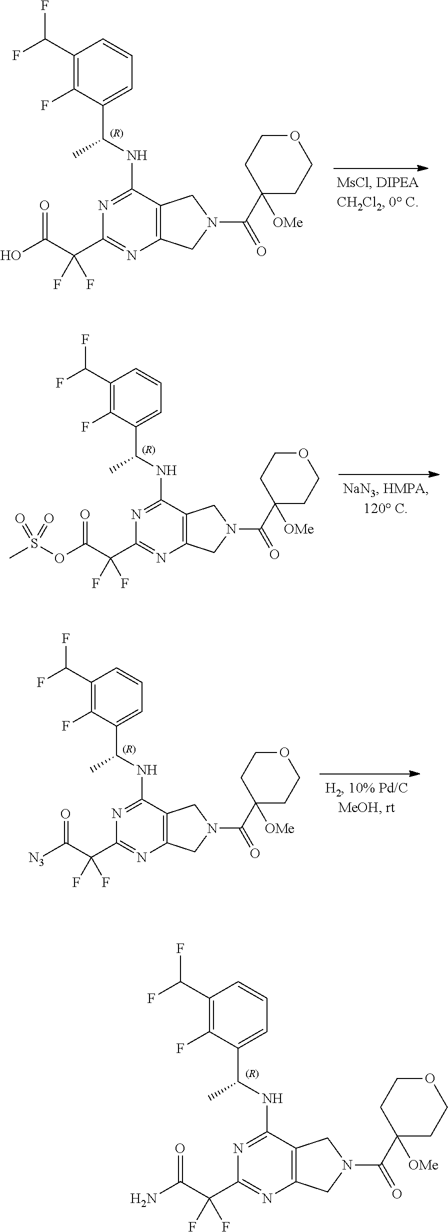

### Step 1.

To a stirred solution of 2-(4-{[(1R)-1-[3-(difluoromethyl)-2-fluorophenyl]ethyl]amino}-6-(4-methoxyoxane-4-carbonyl)-5H,6H,7H-pyrrolo[3,4-d]pyrimidin-2-yl)-2,2-difluoroethan-1-ol (220 mg, 0.41 mmol) and DIPEA (87 μL, 0.50 mmol) in DCM (7.7 mL), methanesulfonyl chloride (35 μL, 0.46 mmol) was added dropwise at 0° C. The reaction mixture was stirred at room temperature for 2 h. The reaction was then quenched with saturated NaHCO₃ solution and extracted with EtOAc. The combined organic layers were dried over Na₂SO₄, then filtered and concentrated under reduced pressure to give 2-(4-{[(1R)-1-[3-(difluoromethyl)-2-fluorophenyl]ethyl]amino}-6-(4-methoxyoxane-4-carbonyl)-5H,6H,7H-pyrrolo[3,4-d]pyrimidin-2-yl)-2,2-difluoroethyl methanesulfonate (280 mg, quantitative). LCMS (ESI): m/z: [M+H] calculated for exact mass for C₂₅H₃₀F₅N₄06S: 609.17. found: 609.05.

### Step 2.

To a stirred solution of 2-(4-{[(1R)-1-[3-(difluoromethyl)-2-fluorophenyl]ethyl]amino}-6-(4-methoxyoxane-4-carbonyl)-5H,6H,7H-pyrrolo[3,4-d]pyrimidin-2-yl)-2,2-difluoroethyl methanesulfonate (192 mg, 0.32 mmol) in HMPA (6.7 mL), sodium azide (103 mg, 1.58 mmol) was added. The reaction mixture was stirred overnight at 120° C., then quenched with water and extracted with diethyl ether. The combined organic layers were dried over Na₂SO₄, filtered, and concentrated under reduced pressure. The resulting material was purified by flash column chromatography to give 2-(2-azido-1,1-difluoroethyl)-N-[(1R)-1-[3-(difluoromethyl)-2-fluorophenyl]ethyl]-6-(4-methoxyoxane-4-carbonyl)-5H,6H,7H-pyrrolo[3,4-d]pyrimidin-4-amine (118 mg, 67% yield). LCMS (ESI): m/z: [M+H] calculated for C₂₄H₂₇F₅N₇O₃: 556.20. found: 556.15.

### Step 3.

To the solution of 2-(2-azido-1,1-difluoroethyl)-N-[(1R)-1-[3-(difluoromethyl)-2-fluorophenyl]-ethyl]-6-(4-methoxyoxane-4-carbonyl)-5H,6H,7H-pyrrolo[3,4-d]pyrimidin-4-amine (105 mg, 0.19 mmol) in MeOH (5.3 mL), 10% palladium on carbon (42 mg) was added. The mixture was degassed, refilled with hydrogen (4×), and stirred over 2 h, then filtered through the pad of Celite® and the filter cake washed with methanol, acetone and DCM. The filtrate was purified by preparative HPLC to give 2-(2-amino-1,1-difluoroethyl)-N-[(1R)-1-[3-(difluoromethyl)-2-fluorophenyl]ethyl]-6-(4-methoxyoxane-4-carbonyl)-5H,6H,7H-pyrrolo[3,4-d]pyrimidin-4-amine (32.9 mg, 33% yield). LCMS (ESI): m/z: [M+H] calculated for C₂₄H₂₉F₅N₅O₃: 530.21. found: 529.81; ¹H NMR (300 MHz, DMSO-d6) δ 8.33-8.20 (m, 1H), 7.62 (t, J=7.5 Hz, 1H), 7.49 (t, J=7.1 Hz, 1H), 7.32-7.24 (m, 1H), 7.22 (t, J=54.4 Hz, 1H), 5.54 (d, J=9.7 Hz, 1H), 4.97-4.81 (m, 2H), 4.70-4.52 (m, 2H), 3.77-3.53 (m, 4H), 3.25-2.98 (m, 5H), 2.08-1.77 (m, 4H), 1.53 (dd, J=7.1, 4.3 Hz, 3H), 1.39 (s, 2H).

### Example 500. Synthesis of 2-[3-(1-{[2-Chloro-6-(morpholine-4-carbonyl)-5H,6H,7H-pyrrolo[3,4-d]pyrimidin-4-yl]amino}ethyl)phenyl]propan-1-ol

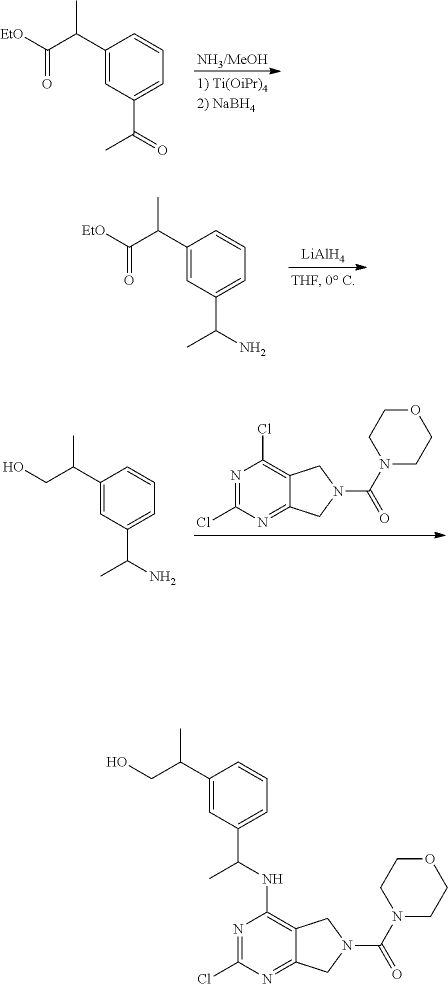 [SMILES: CCOC(=O)C(C)c1cccc(C(C)=O)c1 ; CCOC(=O)C(C)c1cccc(C(C)N)c1 ; "LiAlH4" ; CC(N)c1cccc(C(C)CO)c1 ; O=C(N1CCOCC1)N1Cc2nc(Cl)nc(Cl)c2C1 ; CC(CO)c1cccc(C(C)Nc2nc(Cl)nc3c2CN(C(=O)N2CCOCC2)C3)c1]

### Step 1.

To a mixture of ethyl 2-(3-acetylphenyl)propanoate (400 mg, 1.82 mmol) and 2M NH₃ in MeOH (4.6 mL, 9.2 mmol) at rt was added titanium(IV) isopropoxide (1.1 mL, 3.63 mmol). The mixture was stirred at rt overnight then NaBH₄ (240 mg, 6.4 mmol) added and the mixture stirred at rt for an additional 30 min. The mixture was concentrated under reduced pressure and H₂O added to the residue, then filtered through a pad of Celite® and the filter cake washed with EtOAc and MeOH. The filtrate was concentrated under reduced pressure then diluted with EtOAc and washed with H₂O and brine. The combined aqueous layers were extracted with EtOAc (×4) and the combined organic layers were dried over anhydrous Na₂SO₄, filtered and concentrated under reduced pressure to give ethyl 2-[3-(1-aminoethyl)phenyl]propanoate (360 mg, 90% yield), which was used directly in the next step without further purification. ¹H NMR (300 MHz, DMSO-d₆) δ ppm 7.33-7.03 (m, 4H), 4.15-3.89 (m, 3H), 3.73 (q, J=7.1 Hz, 1H), 1.37 (d, J=7.1 Hz, 3H), 1.22 (d, J=6.6 Hz, 3H), 1.13 (t, J=7.1 Hz, 3H).

### Step 2.

To a mixture of 2M LiAlH₄ in THF (1.44 mL, 2.88 mmol) in THF (19 mL) at 0° C. was added a solution of ethyl 2-[3-(1-aminoethyl)phenyl]propanoate (319 mg, 1.44 mmol) in THF (9.6 mL) over 5 min. The mixture was allowed to warm to rt and was stirred for 1 h, then quenched with H₂O (114 μL) and 15% aqueous NaOH (114 μL). After stirring for 10 min an additional aliquot of H₂O (228 μL) was added. The mixture was stirred for 2 h, filtered and the filter cake washed with THF. The filtrate was concentrated under reduced pressure to give 2-[3-(1-aminoethyl)phenyl]propan-1-ol (240 mg, 93% yield). The product was used in the next step without further purification. H NMR (300 MHz, DMSO-d₆) δ ppm 7.27-6.93 (m, 4H), 4.61 (t, J=5.3 Hz, 1H), 3.93 (q, J=6.6 Hz, 1H), 3.55-3.37 (m, 2H), 2.84-2.69 (m, 1H), 1.76 (br s, 2H), 1.22 (d, J=6.6 Hz, 3H), 1.18 (d, J=7.0 Hz, 3H).

### Step 3.

2-[3-(1-{[2-Chloro-6-(morpholine-4-carbonyl)-5H,6H,7H-pyrrolo[3,4-d]pyrimidin-4-yl]amino}ethyl)phenyl]propan-1-ol was synthesized in a manner similar to 2-chloro-N-{1-[3-(difluoromethyl)-2-fluorophenyl]ethyl}-6-(morpholine-4-carbonyl)-5H,6H,7H-pyrrolo[3,4-d]pyrimidin-4-amine except 1-[3-(difluoromethyl)-2-fluorophenyl]ethan-1-amine was substituted with 2-[3-(1-aminoethyl)phenyl]propan-1-ol to give (227 mg, 53% yield). LCMS (ESI): m/z: [M+H] calculated for C₂₂H₂₈ClN₅O₃: 445.19. found 446.04; ¹H NMR (300 MHz, DMSO-d₆) δ ppm 8.11 (d, J=8.0 Hz, 1H), 7.29-7.16 (m, 3H), 7.13-7.06 (m, 1TH), 5.38-5.17 (m, 1H), 4.64 (t, J=5.2 Hz, 1H), 4.59-4.43 (m, 4H), 3.63 (t, J=4.6 Hz, 4H), 3.55-3.38 (m, 2H), 3.24 (t, J=4.7 Hz, 4H), 2.85-2.71 (m, 1H), 1.48 (d, J=7.0 Hz, 3H), 1.19 (dd, J=7.0, 1.9 Hz, 3H).

### Example 501. Synthesis of 2-[3-(1-{[2-chloro-6-(morpholine-4-carbonyl)-5H,6H,7H-pyrrolo[3,4-d]pyrimidin-4-yl]amino}ethyl)phenyl]ethan-1-ol

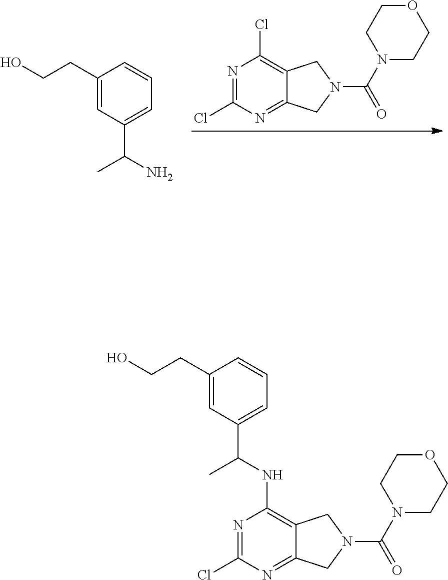 [SMILES: CC(N)c1cccc(CCO)c1 ; O=C(N1CCOCC1)N1Cc2nc(Cl)nc(Cl)c2C1 ; CC(Nc1nc(Cl)nc2c1CN(C(=O)N1CCOCC1)C2)c1cccc(CCO)c1]

### Step 1.

2-[3-(1-{[2-chloro-6-(morpholine-4-carbonyl)-5H,6H,7H-pyrrolo[3,4-d]pyrimidin-4-yl]amino}ethyl)phenyl]ethan-1-ol was synthesized in a manner similar to 2-chloro-N-{1-[3-(difluoromethyl)-2-fluorophenyl]ethyl}-6-(morpholine-4-carbonyl)-5H,6H,7H-pyrrolo[3,4-d]pyrimidin-4-amine except 1-[3-(difluoromethyl)-2-fluorophenyl]ethan-1-amine was substituted with 2-[3-(1-aminoethyl)phenyl]ethan-1-ol to give (226 mg, 46% yield). LCMS (ESI): m/z: [M+H] calculated for C₂₁H₂₆ClN₅O₃: 431.17. found 432.00; ¹H NMR (300 MHz, DMSO-d₆) δ ppm 8.10 (d, J=8.0 Hz, 1H), 7.29-7.15 (m, 3H), 7.14-7.05 (m, 1H), 5.27 (t, J=7.3 Hz, 1H), 4.65 (t, J=5.2 Hz, 1H), 4.53 (d, J=13.0 Hz, 4H), 3.68-3.53 (m, 6H), 3.24 (t, J=4.6 Hz, 4H), 2.72 (t, J=7.1 Hz, 2H), 1.48 (d, J=7.0 Hz, 3H).

### Example 502. Synthesis of 4-{[(1R)-1-[3-amino-5-(trifluoromethyl)phenyl]ethyl]amino}-8-methyl-6-(morpholin-4-yl)-5H,6H,7H,8H-pyrido[2,3-d]pyrimidin-7-one

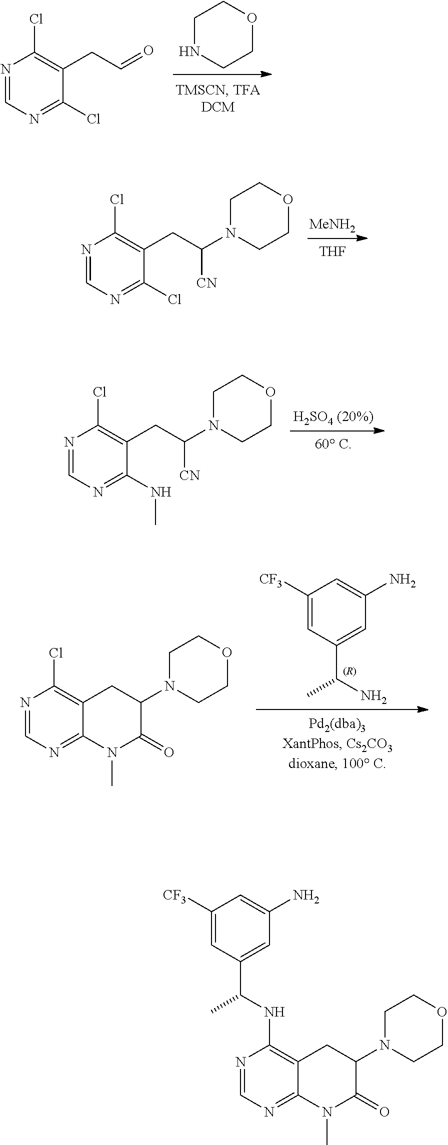

### Step 1.

To a mixture of 2-(4,6-dichloropyrimidin-5-yl)acetaldehyde (6.0 g, 31.4 mmol) in DCM (180 mL) at −15° C. under an atmosphere of Ar was added morpholine (2.9 mL, 32.9 mmol) and TFA (7.2 mL, 94.2 mmol). The mixture was warmed to rt and stirred overnight then TMSCN (7.9 mL, 62.8 mmol) was added and the mixture stirred at rt overnight. H₂O and K₂CO₃ were added and the mixture was extracted with DCM. The organic layer was dried, filtered, concentrated under reduced pressure and the crude residue was purified by column chromatography to give 3-(4,6-dichloropyrimidin-5-yl)-2-(morpholin-4-yl)propanenitrile (3.7 g, 41% yield). LCMS (ESI): m/z: [M+H] calculated for C₁₁H₁₂Cl₂N₄O: 286.04. found 287.05; ¹H NMR (300 MHz, CDCl₃) δ ppm 8.76 (s, 1H), 3.98 (t, J=8.0 Hz, 1H), 3.80-3.70 (m, 4H), 3.41 (dd, J=8.0, 1.8 Hz, 2H), 2.88 (dt, J=10.1, 4.6 Hz, 2H), 2.57 (dt, J=10.3, 4.5 Hz, 2H).

### Step 2.

To a mixture of 3-(4,6-dichloropyrimidin-5-yl)-2-(morpholin-4-yl)propanenitrile (4.10 g, 14.3 mmol) in THF (123 mL) under an atmosphere of Ar was added 2M MeNH₂ in THF (17.8 mL, 35.7 mmol). The mixture was stirred at rt overnight, then partitioned between H₂O and EtOAc. The organic layer was dried, filtered and the filtrate concentrated under reduced pressure to give 3-[4-chloro-6-(methylamino)pyrimidin-5-yl]-2-(morpholin-4-yl)propanenitrile (4.27 gg, 100% yield). LCMS (ESI): m/z: [M+H] calculated for C₁₂H₁₆ClN₅O: 281.10. found 282.00; ¹H NMR (300 MHz, DMSO-d₆) δ ppm 8.23 (s, 1H), 7.54 (d, J=4.8 Hz, 1H), 4.10-3.97 (m, 2H), 3.68-3.55 (m, 4H), 3.16 (dd, J=14.6, 8.7 Hz, 1H), 2.98 (dd, J=14.6, 7.9 Hz, 1H), 2.87 (d, J=4.4 Hz, 3H), 2.77-2.65 (m, 2H), 1.99 (s, 1H).

### Step 3.

A mixture of 3-[4-chloro-6-(methylamino)pyrimidin-5-yl]-2-(morpholin-4-yl)propanenitrile (2.0 g, 7.1 mmol) and 20% solution of H₂SO₄ (85 mL) was stirred at 50° C. overnight, then at 60° C. for a further 1 day. H₂O and NaHSO₃ were added and the mixture was extracted with EtOAc. The organic layer was concentrated under reduced pressure and the crude product was purified by column chromatography to give 4-chloro-8-methyl-6-(morpholin-4-yl)-5H,6H,7H,8H-pyrido[2,3-d]pyrimidin-7-one (287 mg, 14% yield). LCMS (ESI): m/z: [M+H] calculated for C₁₂H₁₅ClN₄O₂: 282.09. found 283.10; H NMR (300 MHz, CDCl₃) δ ppm 8.65 (s, 1H), 3.69 (t, J=4.4 Hz, 4H), 3.47 (s, 3H), 3.44 (dd, J=7.6, 5.7 Hz, 1H), 3.28 (dd, J=16.9, 7.7 Hz, 1H), 3.12 (dd, J=17.0, 5.6 Hz, 1H), 2.69 (t, J=4.9 Hz, 4H).

### Step 4.

To a mixture of 4-chloro-8-methyl-6-(morpholin-4-yl)-5H,6H,7H,8H-pyrido[2,3-d]pyrimidin-7-one (159 mg, 0.56 mmol) and 3-[(1R)-1-aminoethyl]-5-(trifluoromethyl)aniline HCl salt (162 mg, 0.68 mmol) in 1,4-dioxane (4.8 mL) in a sealed tube was added Cs₂CO₃ (458 mg, 1.40 mmol). The mixture was purged with Ar for 10 min, then Pd₂(dba)₃ (26 mg, 0.28 mmol) and XantPhos (33 mg, 0.56 mmol) were added. The mixture was heated to 100° C. and stirred overnight, then filtered through Celite® and the filtrate extracted with EtOAc. The organic layer was dried over anhydrous Na₂SO₄, filtered, the filtrate concentrated under reduced pressure and the crude residue was purified by preparative HPLC to give 4-{[(1R)-1-[3-amino-5-(trifluoromethyl)phenyl]ethyl]amino}-8-methyl-6-(morpholin-4-yl)-5H,6H,7H,8H-pyrido[2,3-d]pyrimidin-7-one (102 mg, 40% yield). LCMS (ESI): m/z: [M+H] calculated for C₂₁H₂₆F₃N₆O₂: 450.20. found 451.15; H NMR (300 MHz, CDCl₃) δ ppm 8.54 (s, 1H), 7.90 (s, 1H), 7.75 (s, 1H), 7.37 (s, 1H), 6.53 (s, 1H), 4.26 (q, J=6.5 Hz, 1H), 3.74 (t, J=4.6 Hz, 4H), 3.57 (t, J=8.1 Hz, 1H), 3.47 (s, 3H), 2.92 (d, J=8.1 Hz, 2H), 2.80 (dt, J=4.8 Hz, 4H), 2.20 (s, 1H), 1.46 (d, J=6.6 Hz, 3H).

### Example 503. Synthesis of N-[(1R)-1-[3-amino-5-(trifluoro-methyl)phenyl]ethyl]-2-[(methylamino)methyl]-6-(morpholine-4-carbonyl)-5H,6H,7H-pyrrolo[3,4-d]-pyrimidin-4-amine

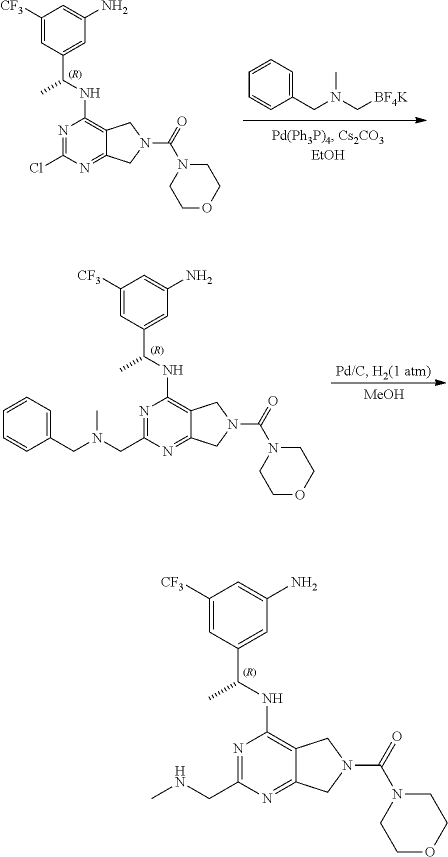

### Step 1.

A mixture of N-[(1R)-1-[3-amino-5-(trifluoromethyl)phenyl]ethyl]-2-chloro-6-(morpholine-4-carbonyl)-5H,6H,7H-pyrrolo[3,4-d]pyrimidin-4-amine (600 mg, 1.27 mmol), potassium N-benzyl-N-methyl-aminomethyltrifluoroborate (399 mg, 1.66 mmol) and Cs₂CO₃ (623 mg, 1.91 mmol) in EtOH (21.2 mL) was purged with Ar. Pd(Ph₃P)₄ (147 mg, 0.13 mmol) was added and the mixture was heated to 135° C. and stirred overnight. The mixture was filtered through a pad of Celite® and the filtrate was concentrated under reduced pressure, then partitioned between EtOAc and H₂O. The aqueous layer was extracted with EtOAc (×4) and the combined organic layers were dried over Na₂SO₄, filtered, concentrated under reduced pressure and the crude residue was purified by column chromatography to give N-[(1R)-1-[3-amino-5-(trifluoromethyl)phenyl]ethyl]-2-{[benzyl(methyl)amino]methyl}-6-(morpholine-4-carbonyl)-5H,6H,7H-pyrrolo[3,4-d]pyrimidin-4-amine (425 mg, 59% yield). LCMS (ESI): m/z: [M+H] calculated for C₂₉H₃₄F₃N₇O₂: 569.27. found 570.25; H NMR (300 MHz, DMSO-d₆) δ ppm 7.58 (d, J=7.6 Hz, 1H), 7.34-7.12 (m, 5H), 6.79 (d, J=7.3 Hz, 2H), 6.65 (s, 1H), 5.49 (d, J=5.5 Hz, 2H), 5.29 (t, J=7.3 Hz, 1H), 4.68-4.43 (m, 4H), 3.63 (t, J=4.6 Hz, 4H), 3.52 (s, 2H), 3.49 (d, J=2.0 Hz, 2H), 3.25 (t, J=4.7 Hz, 4H), 2.09 (s, 3H), 1.46 (d, J=7.0 Hz, 3H).

### Step 2.

A mixture of N-[(1R)-1-[3-amino-5-(trifluoromethyl)phenyl]ethyl]-2-{[benzyl(methyl)-amino]methyl}-6-(morpholine-4-carbonyl)-5H,6H,7H-pyrrolo[3,4-d]pyrimidin-4-amine (425 mg, 0.74 mmol) in MeOH (19.1 mL) under an atmosphere of Ar was evacuated and refilled (×3). Pd on carbon (20 wt %, 85 mg) was added under an atmosphere of Ar, the flask was evacuated and the mixture was placed under an atmosphere of H₂ (balloon). The mixture was stirred at rt overnight, then filtered through a pad of Celite®, the filtrate was concentrated under reduced pressure and the crude residue was purified by preparative HPLC to give N-[(1R)-1-[3-amino-5-(trifluoro-methyl)phenyl]ethyl]-2-[(methylamino)methyl]-6-(morpholine-4-carbonyl)-5H,6H,7H-pyrrolo[3,4-d]-pyrimidin-4-amine (115 mg, 35% yield). LCMS (ESI): m/z: [M+H] calculated for C22H₂₈F₃N₇O₂: 479.23. found 480.09; ¹H NMR (300 MHz, DMSO-d₆) δ ppm 7.62 (d, J=7.5 Hz, 1H), 6.80 (d, J=12.8 Hz, 2H), 6.67 (d, J=1.9 Hz, 1H), 5.51 (s, 2H), 5.24 (t, J=7.3 Hz, 1H), 4.68-4.40 (m, 4H), 3.63 (t, J=4.6 Hz, 4H), 3.58-3.38 (m, 2H), 3.24 (t, J=4.6 Hz, 4H), 2.14 (s, 3H), 1.44 (d, J=7.0 Hz, 3H).

### Example 504. Synthesis of N-[(1R)-1-[3-amino-5-(trifluoromethyl)phenyl]ethyl]-2-[(morpholin-4-yl)-methyl]-6-(morpholine-4-carbonyl)-5H,6H,7H-pyrrolo[3,4-d]pyrimidin-4-amine

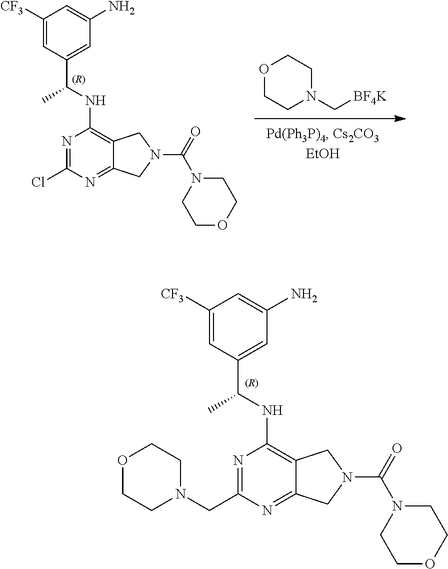

### Step 1.

N-[(1R)-1-[3-amino-5-(trifluoromethyl)phenyl]ethyl]-2-[(morpholin-4-yl)-methyl]-6-(morpholine-4-carbonyl)-5H,6H,7H-pyrrolo[3,4-d]pyrimidin-4-amine was synthesized in a manner similar to N-[(1R)-1-[3-amino-5-(trifluoromethyl)phenyl]ethyl]-2-{[benzyl(methyl)amino]methyl}-6-(morpholine-4-carbonyl)-5H,6H,7H-pyrrolo[3,4-d]pyrimidin-4-amine except potassium N-benzyl-N-methyl-aminomethyltrifluoroborate was substituted with potassium (morpholin-4-yl)methyltrifluoroborate to give (61 mg, 31% yield). LCMS (ESI): m/z: [M+H] calculated for C₂₅H32F₃N₇O₃: 535.25. found 536.1; H NMR (300 MHz, METHANOL-d₄) δ ppm 6.85 (s, 2H), 6.75 (s, 1H), 5.34 (q, J=7.0 Hz, 1H), 4.72-4.64 (m, 2H), 4.61 (t, 2H), 3.74 (t, J=4.7 Hz, 4H), 3.57 (t, 4H), 3.38 (t, J=4.7 Hz, 4H), 2.41 (dt, J=9.2, 5.0 Hz, 4H), 1.54 (d, J=7.1 Hz, 3H).

The following example 539 was synthesized in the manner similar to Example xx.

| | | Mass |
|---|---|---|
| Example # | Structure | Found |
| Example 539. | 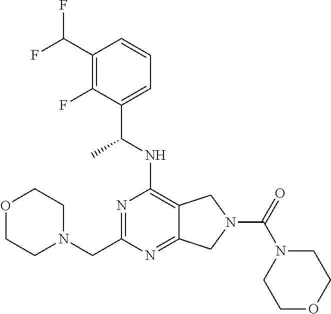 [SMILES: C[C@@H](Nc1nc(CN2CCOCC2)nc2c1CN(C(=O)N1CCOCC1)C2)c1cccc(C(F)F)c1F] | 521.0 |

### Example 505. Synthesis of N4-[(1R)-1-[3-(difluoromethyl)-2-fluorophenyl]ethyl]-N2-methyl-6-(morpholine-4-carbonyl)-5H,6H,7H-pyrrolo[3,4-d]pyrimidine-2,4-diamine

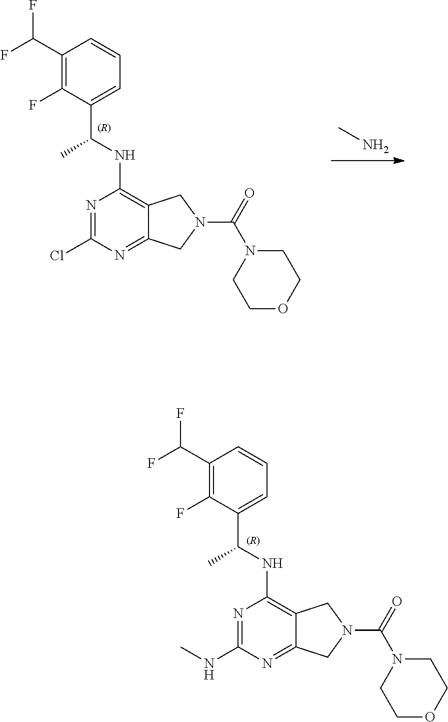

### Step 1.

N4-[(1R)-1-[3-(difluoromethyl)-2-fluorophenyl]ethyl]-N2-methyl-6-(morpholine-4-carbonyl)-5H,6H,7H-pyrrolo[3,4-d]pyrimidine-2,4-diamine was synthesized in a manner similar to N2-methyl-6-(morpholine-4-carbonyl)-N4-[(1R)-1-[3-nitro-5-(trifluoromethyl)phenyl]ethyl]-5H,6H,7H-pyrrolo[3,4-d]pyrimidine-2,4-diamine except 2-chloro-6-(morpholine-4-carbonyl)-N-[(1R)-1-[3-nitro-5-(trifluoromethyl)phenyl]-ethyl]-5H,6H,7H-pyrrolo[3,4-d]pyrimidin-4-amine was substituted with 2-chloro-N-[(1R)-1-[3-(difluoromethyl)-2-fluorophenyl]ethyl]-6-(morpholine-4-carbonyl)-5H,6H,7H-pyrrolo[3,4-d]pyrimidin-4-amine to give (156 mg, 63% yield). LCMS (ESI): m/z: [M+H] calculated for C₂₁H₂₅F₃N₆O₂: 450.20. found 450.97; ¹H NMR (300 MHz, DMSO-d₆) δ ppm 7.59 (t, J=7.4 Hz, 1H), 7.47 (t, J=7.1 Hz, 1H), 7.36-7.24 (m, 2H), 7.20 (t, J=54.4 Hz, 1H), 6.38-6.18 (m, 1H), 5.54 (p, J=7.0 Hz, 1H), 4.48 (q, J=13.3 Hz, 2H), 4.31 (s, 2H), 3.63 (t, 4H), 3.23 (t, J=4.6 Hz, 4H), 2.59 (d, J=4.6 Hz, 3H), 1.46 (d, J=7.0 Hz, 3H).

### Example 506. Synthesis of 2-[2-chloro-3-(1-{[2-chloro-6-(morpholine-4-carbonyl)-5H,6H,7H-pyrrolo[3,4-d]pyrimidin-4-yl]amino}ethyl)phenyl]-2,2-difluoro-ethan-1-ol

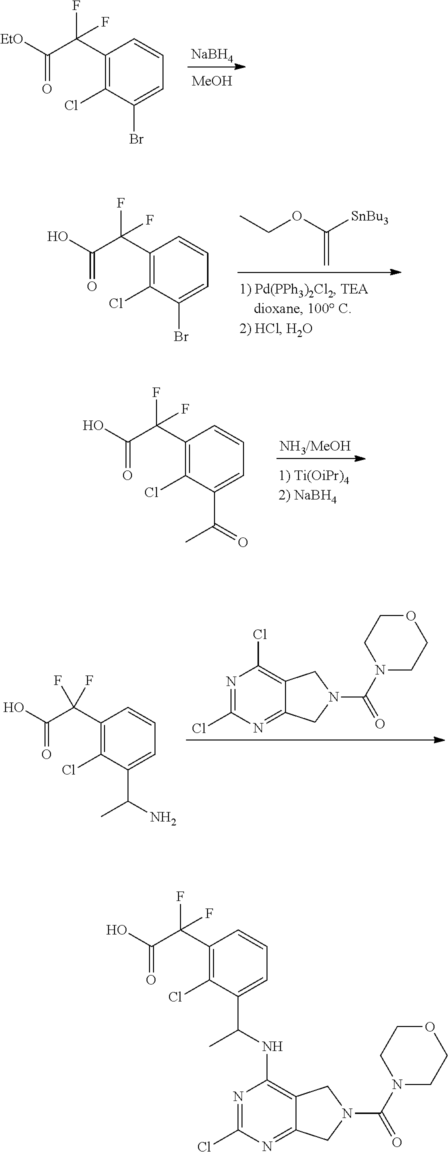

### Step 1.

To a mixture of ethyl 2-(3-bromo-2-chlorophenyl)-2,2-difluoroacetate (1.06 g, 3.38 mmol) in MeOH (21.8 mL) at 0° C. was added NaBH₄ (256 mg, 6.76 mmol). The mixture was warmed to rt and stirred for 2 h, then aqueous NH₄Cl added and the mixture extracted with Et₂O (×3). The combined organic layers were dried over Na₂SO₄, filtered, the filtrate concentrated under reduced pressure and the crude residue was purified by column chromatography to give 2-(3-bromo-2-chlorophenyl)-2,2-difluoroethan-1-ol (345 mg, 38% yield). ¹H NMR (300 MHz, DMSO-d₆) δ ppm 8.15-7.78 (m, 1H), 7.65 (dd, J=7.9, 1.6 Hz, 1H), 7.42 (t, J=7.9 Hz, 1H), 5.70 (t, J=6.5 Hz, 1H), 4.02 (td, J=14.2, 6.5 Hz, 2H).

### Step 2.

A mixture of 2-(3-bromo-2-chlorophenyl)-2,2-difluoroethan-1-ol (345 mg, 1.27 mmol) and Et₃N (350 μL, 2.54 mmol) in 1,4-dioxane (3.9 mL) in a pressure tube was degassed with Ar. Tributyl(1-ethoxyvinyl)tin (558 μL, 1.65 mmol) and Pd(PPh₃)₂Cl₂ (89 mg, 0.13 mmol) were added and the mixture was heated to 80° C. and stirred overnight. After cooling, 1M HCl was added, the mixture was stirred for 1 h, then the aqueous and organic layers were separated. 1M KF solution was added to the organic layer and the mixture stirred vigorously for 1 h, then filtered through a pad of Celite®. The aqueous and organic layers were partitioned and the procedure with 1M KF was repeated. The organic layer was dried over Na₂SO₄, filtered, the filtrate concentrated under reduced pressure and the crude residue was purified by column chromatography to give 1-[2-chloro-3-(1,1-difluoro-2-hydroxyethyl)phenyl]ethan-1-one (161 mg, 54% yield). LCMS (ESI): m/z: [M+H] calculated for C₁₀H9ClF₂O₂: 234.03. found 234.95; ¹H NMR (300 MHz, DMSO-d₆) δ ppm 7.80-7.64 (m, 2H), 7.56 (t, J=7.7 Hz, 1H), 5.71 (t, J=6.5 Hz, 1H), 4.02 (td, J=14.3, 6.5 Hz, 2H), 2.58 (s, 3H).

### Step 3.

2-[3-(1-aminoethyl)-2-chlorophenyl]-2,2-difluoroethan-1-ol was synthesized in a manner similar to ethyl 2-[3-(1-aminoethyl)phenyl]propanoate except ethyl 2-(3-acetylphenyl)propanoate was substituted with 1-[2-chloro-3-(1,1-difluoro-2-hydroxyethyl)phenyl]ethan-1-one to give (123 mg, 76% yield).

### Step 4.

2-[2-Chloro-3-(1-{[2-chloro-6-(morpholine-4-carbonyl)-5H,6H,7H-pyrrolo[3,4-d]pyrimidin-4-yl]amino}ethyl)phenyl]-2,2-difluoro-ethan-1-ol was synthesized in a manner similar to 2-chloro-N-{1-[3-(difluoromethyl)-2-fluorophenyl]ethyl}-6-(morpholine-4-carbonyl)-5H,6H,7H-pyrrolo[3,4-d]pyrimidin-4-amine except 1-[3-(difluoromethyl)-2-fluorophenyl]ethan-1-amine was substituted with 2-[3-(1-aminoethyl)-2-chlorophenyl]-2,2-difluoroethan-1-ol to give (56 mg, 32% yield). LCMS (ESI): m/z: [M+H] calculated for C₂₁H₂₃ClF₂N5O₃: 520.24. found 521.0; ¹H NMR (300 MHz, DMSO-d₆) δ ppm 8.28 (d, J=7.5 Hz, 1H), 7.64 (d, J=7.5 Hz, 1H), 7.52 (dd, J=7.9, 1.8 Hz, 1H), 7.44 (t, J=7.7 Hz, 1H), 5.67 (dt, J=13.9, 6.7 Hz, 2H), 4.69-4.42 (m, 4H), 4.03 (tt, J=13.7, 6.4 Hz, 2H), 3.63 (t, J=4.6 Hz, 4H), 3.25 (t, J=4.6 Hz, 4H), 1.47 (d, J=6.9 Hz, 3H).

### Example 507. Synthesis of 2-[3-(1-{[2-chloro-6-(morpholine-4-carbonyl)-5H,6H,7H-pyrrolo[3,4-d]pyrimidin-4-yl]amino}ethyl)-2-methylphenyl]-2,2-difluoroethan-1-ol

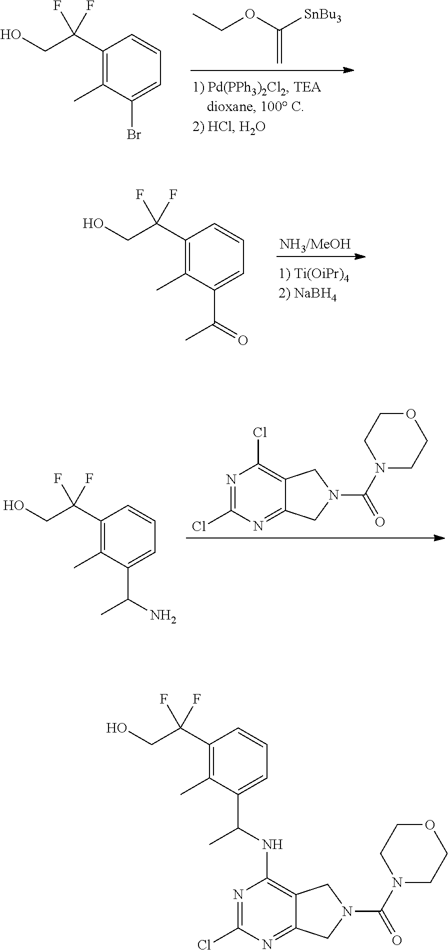

### Step 1.

1-[3-(1,1-Difluoro-2-hydroxyethyl)-2-methylphenyl]ethan-1-ol was synthesized in a manner similar to 1-[2-chloro-3-(1,1-difluoro-2-hydroxyethyl)phenyl]ethan-1-one except 2-(3-bromo-2-chlorophenyl)-2,2-difluoroethan-1-ol was substituted with 2-(3-bromo-2-methylphenyl)-2,2-difluoroethan-1-ol to give (482 mg, 56% yield). ¹H NMR (300 MHz, DMSO-d₆) δ ppm 7.72 (d, J=7.6 Hz, 1H), 7.59 (dd, J=8.0, 1.4 Hz, 1H), 7.41 (t, J=7.8 Hz, 2H), 5.70 (t, J=6.5 Hz, 1H), 3.91 (td, J=14.4, 6.4 Hz, 2H), 2.56 (s, 3H), 2.38 (t, J=2.2 Hz, 3H).

### Step 2.

2-[3-(1-Aminoethyl)-2-methylphenyl]-2,2-difluoroethan-1-ol was synthesized in a manner similar to ethyl 2-[3-(1-aminoethyl)phenyl]propanoate except ethyl 2-(3-acetylphenyl)propanoate was substituted with 1-[3-(1,1-difluoro-2-hydroxyethyl)-2-methylphenyl]ethan-1-ol to give (211 mg, 93% yield), which was used directly in the next step.

### Step 3.

2-[3-(1-{[2-Chloro-6-(morpholine-4-carbonyl)-5H,6H,7H-pyrrolo[3,4-d]pyrimidin-4-yl]amino}ethyl)-2-methylphenyl]-2,2-difluoroethan-1-ol was synthesized in a manner similar to 2-chloro-N-{1-[3-(difluoromethyl)-2-fluorophenyl]ethyl}-6-(morpholine-4-carbonyl)-5H,6H,7H-pyrrolo[3,4-d]pyrimidin-4-amine except 1-[3-(difluoromethyl)-2-fluorophenyl]ethan-1-amine was substituted with 2-[3-(1-aminoethyl)-2-methylphenyl]-2,2-difluoroethan-1-ol to give (118 mg, 50% yield). LCMS (ESI): m/z: [M+H] calculated for C₂₂H₂₆ClF₂N₅O₃: 481.17. found 481.66; ¹H NMR (300 MHz, METHANOL-d₄) δ ppm 7.50 (d, J=7.7 Hz, 1H), 7.43 (d, J=7.6 Hz, 1H), 7.24 (t, J=7.8 Hz, 1H), 5.61 (q, J=6.9 Hz, 1H), 4.67-4.50 (m, 4H), 3.96 (td, J=13.9, 4.4 Hz, 2H), 3.76-3.68 (m, 4H), 3.40-3.33 (m, 4H), 2.57 (s, 3H), 1.53 (d, J=6.9 Hz, 3H); ¹H NMR (300 MHz, DMSO-d₆) δ ppm 8.26 (d, J=7.4 Hz, 1H), 7.55 (d, J=7.6 Hz, 1H), 7.37 (dd, J=7.9, 1.5 Hz, 1H), 7.28 (t, J=7.7 Hz, 1H), 5.68 (t, J=6.4 Hz, 1H), 5.55-5.39 (m, 1H), 4.63-4.40 (m, 4H), 3.89 (td, J=14.7, 6.3 Hz, 2H), 3.63 (t, J=4.6 Hz, 3H), 3.24 (t, J=4.8 Hz, 4H), 1.45 (d, J=6.9 Hz, 3H).

### Example 508. Synthesis of 2-[(cyclopropylamino)methyl]-N-{1-[3-(difluoromethyl)-2-fluorophenyl]ethyl}-6-(morpholine-4-carbonyl)-5H,6H,7H-pyrrolo[3,4-d]pyrimidin-4-amine

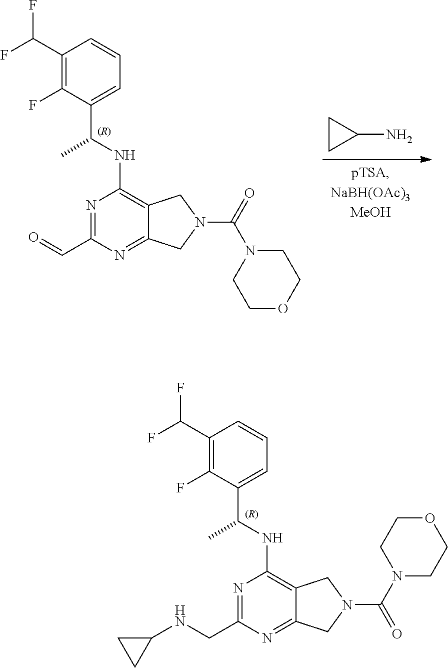

### Step 1.

To a mixture of 4-({1-[3-(difluoromethyl)-2-fluorophenyl]ethyl}amino)-6-(morpholine-4-carbonyl)-5H,6H,7H-pyrrolo[3,4-d]pyrimidine-2-carbaldehyde (200 mg, 0.44 mmol) in MeOH (4 mL) at rt was added cyclopropylamine (40 μL, 0.58 mmol) and pTSA (7.7 mg, 0.04 mmol). The mixture was stirred at rt overnight, then NaBH(OAc)₃ (28 mg, 0.44 mmol) was added and the mixture was stirred at rt for an additional 30 min. The mixture was concentrated under reduced pressure and the residue was diluted with saturated NH₄Cl and extracted with EtOAc. The combined organic layers were dried over anhydrous MgSO₄, filtered, the filtrate was concentrated under reduced pressure and the residue was purified by preparative HPLC to give 2-[(cyclopropylamino)methyl]-N-{1-[3-(difluoromethyl)-2-fluorophenyl]ethyl}-6-(morpholine-4-carbonyl)-5H,6H,7H-pyrrolo[3,4-d]pyrimidin-4-amine (41 mg, 19% yield). LCMS (ESI): m/z: [M+H] calculated for C₂₄H₂₉F₃N₆O₂: 490.23. found 491.01; ¹H NMR (300 MHz, DMSO-dL₆) δ ppm 7.79 (d, J=7.0 Hz, 1H), 7.66-7.55 (m, 1H), 7.47 (t, J=7.1 Hz, 1H), 7.26 (t, J=7.7 Hz, 1H), 7.21 (t, J=54.4 Hz, 1H), 5.56 (q, J=7.0 Hz, 1H), 4.71-4.45 (m, 4H), 3.75-3.59 (m, 4H), 3.54 (d, J=6.3 Hz, 2H), 3.25 (t, J=4.6 Hz, 4H), 2.45-2.35 (s, 1H), 1.89-1.78 (m, 1H), 1.51 (d, J=7.0 Hz, 3H), 0.31-−0.01 (m, 4H); ¹H NMR (300 MHz, METHANOL-d₄) δ ppm 7.57 (t, J=7.4 Hz, 1H), 7.48 (t, J=7.0 Hz, 1H), 7.24 (t, J=7.8 Hz, 1H), 7.02 (t, J=54.9 Hz, 1H), 5.66 (q, J=7.1 Hz, 1H), 4.71 (s, 2H), 4.62 (s, 2H), 3.87-3.60 (m, 6H), 3.50-3.37 (m, 4H), 1.99-1.85 (m, 1H), 1.62 (d, J=7.1 Hz, 3H), 0.46-0.25 (m, 4H).

### Example 509. Synthesis of N-[(1R)-1-[3-(difluoromethyl)-2-fluorophenyl]ethyl]-6-(morpholine-4-carbonyl)-2-{[(oxetan-3-yl)amino]methyl}-5H,6H,7H-pyrrolo-[3,4-d]pyrimidin-4-amine

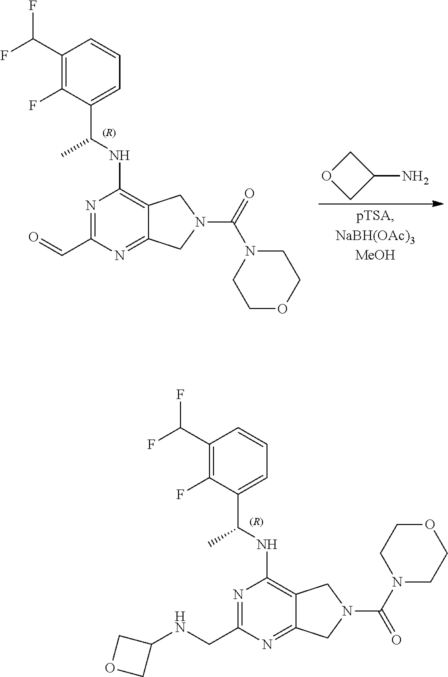

### Step 1.

N-[(1R)-1-[3-(difluoromethyl)-2-fluorophenyl]ethyl]-6-(morpholine-4-carbonyl)-2-{[(oxetan-3-yl)amino]methyl}-5H,6H,7H-pyrrolo-[3,4-d]pyrimidin-4-amine was synthesized in a manner similar to 2-[(cyclopropylamino)methyl]-N-{1-[3-(difluoromethyl)-2-fluorophenyl]ethyl}-6-(morpholine-4-carbonyl)-5H,6H,7H-pyrrolo[3,4-d]pyrimidin-4-amine except cyclopropylamine was substituted with oxetan-3-amine HCl salt and Et₃N to give (11 mg, 59% yield). LCMS (ESI): m/z: [M+H] calculated for C₂₄H₂₉F₃N₆O₃: 506.23. found 507.03; ¹H NMR (300 MHz, DMSO-d₆) δ ppm 7.80 (d, J=7.1 Hz, 1H), 7.62 (t, J=7.4 Hz, 1H), 7.50 (t, J=7.2 Hz, 1H), 7.29 (t, J=7.7 Hz, 1H), 7.23 (t, J=54.4 Hz, 1H), 5.65-5.51 (m, 1H), 4.76-4.46 (m, 4H), 4.38 (dt, J=13.1, 6.6 Hz, 2H), 4.08 (dt, J=18.6, 6.2 Hz, 2H), 3.74 (t, J=6.4 Hz, 1H), 3.64 (t, 4H), 3.47 (s, 2H), 3.26 (t, J=4.7 Hz, 4H), 1.75 (s, 1H), 1.51 (d, J=7.0 Hz, 3H).

### Example 510. Synthesis of 4-{[(1R)-1-[3-(difluoromethyl)-2-fluorophenyl]ethyl]-amino}-6-(4-methoxyoxane-4-carbonyl)-5H,6H,7H-pyrrolo[3,4-d]pyrimidine-2-carboxamide

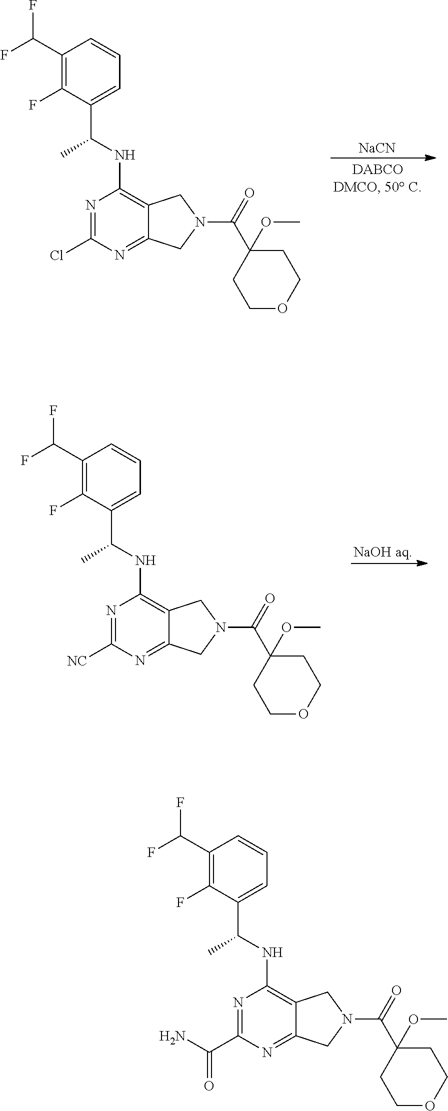 [SMILES: COC1(C(=O)N2Cc3nc(Cl)nc(N[C@H](C)c4cccc(C(F)F)c4F)c3C2)CCOCC1 ; "NaCN" ; COC1(C(=O)N2Cc3nc(C#N)nc(N[C@H](C)c4cccc(C(F)F)c4F)c3C2)CCOCC1 ; "NaOH aq." ; COC1(C(=O)N2Cc3nc(C(N)=O)nc(N[C@H](C)c4cccc(C(F)F)c4F)c3C2)CCOCC1]

### Step 1.

A mixture of 2-chloro-N-[(1R)-1-[3-(difluoromethyl)-2-fluorophenyl]ethyl]-6-(4-methoxyoxane-4-carbonyl)-5H,6H,7H-pyrrolo[3,4-d]pyrimidin-4-amine (300 mg, 0.62 mmol), 5050 (61 mg, 1.2 mmol), DABCO (69 mg, 0.62 mmol) in DMSO (6 mL) and isopropanol (3 mL) was heated to 50° C. and stirred overnight. The mixture was reduced in volume to remove isopropanol and then extracted with Et₂O (×3). The combined organic layers were dried over Na₂SO₄, filtered and the filtrate concentrated under reduced pressure to give 4-{[(1R)-1-[3-(difluoromethyl)-2-fluorophenyl]-ethyl]amino}-6-(4-methoxyoxane-4-carbonyl)-5H,6H,7H-pyrrolo[3,4-d]pyrimidine-2-carbonitrile (330 mg, 100% yield). LCMS (ESI): m/z: [M+H] calculated for C₂₃H₂₄F₃N₅O₃: 475.18. found 476.18; ¹H NMR (300 MHz, DMSO-d₆) δ ppm 8.64-8.49 (m, 1H), 7.65 (t, J=7.6 Hz, 1H), 7.54 (t, J=7.2 Hz, 1H), 7.33 (td, J=7.7, 3.2 Hz, 1H), 7.24 (t, J=54.2 Hz, 1H), 5.62-5.46 (m, 1H), 4.90 (d, J=27.2 Hz, 2H), 4.72-4.48 (m, 2H), 3.77-3.54 (m, 4H), 3.32 (d, J=7.2 Hz, 2H), 3.17 (d, J=13.3 Hz, 2H), 2.01-1.79 (m, 4H), 1.53 (dd, J=7.0, 4.5 Hz, 3H).

### Step 2.

To a mixture of 4-{[(1R)-1-[3-(difluoromethyl)-2-fluorophenyl]ethyl]amino}-6-(4-methoxy-oxane-4-carbonyl)-5H,6H,7H-pyrrolo[3,4-d]pyrimidine-2-carbonitrile (325 mg, 0.68 mmol) in THF (6.5 mL) was added NaOH (109 mg, 2.7 mmol) and H₂O (1.3 mL). The mixture was cooled to 0° C. and H₂O₂(279 μL, 2.7 mmol) was added dropwise, then allowed to warm to rt and stirred overnight. H₂O was added, the mixture was stirred for 5 min and extracted with EtOAc (×4). The combined organic layers were dried over anhydrous Na₂SO₄, filtered, the filtrate concentrated under reduced pressure and the crude residue was purified by preparative HPLC to give 4-{[(1R)-1-[3-(difluoromethyl)-2-fluorophenyl]ethyl]-amino}-6-(4-methoxyoxane-4-carbonyl)-5H,6H,7H-pyrrolo[3,4-d]pyrimidine-2-carboxamide (151 mg, 45% yield). LCMS (ESI): m/z: [M+H] calculated for C₂₃H₂₆F₃N₅O₄: 493.19. found 494.00; H NMR (300 MHz, METHANOL-d₄) δ ppm 7.62 (t, J=7.7 Hz, 1H), 7.48 (t, J=7.3 Hz, 1H), 7.25 (t, J=7.7 Hz, 1H), 6.99 (t, J=54.8 Hz, 1H), 5.71 (q, J=7.0 Hz, 1H), 5.02 (d, J=30.2 Hz, 2H), 4.72 (d, J=13.0 Hz, 2H), 3.85-3.70 (m, 4H), 3.29 (s, 1H), 3.25 (s, 2H), 2.18-1.92 (m, 4H), 1.63 (d, J=7.0 Hz, 3H); ¹H NMR (300 MHz, DMSO-d₆) δ ppm 8.15 (t, J=8.7 Hz, 1H), 7.68 (d, J=7.4 Hz, 2H), 7.59 (s, 1H), 7.51 (t, J=7.2 Hz, 1H), 7.31 (dt, J=7.6, 3.8 Hz, 1H), 7.21 (t, J=54.4 Hz, 1H), 5.73 (t, J=7.1 Hz, 1H), 4.89 (d, J=24.0 Hz, 2H), 4.61 (d, J=22.3 Hz, 2H), 3.63 (d, J=18.7 Hz, 4H), 3.18 (d, J=12.3 Hz, 3H), 2.03-1.79 (m, 4H), 1.57-1.49 (m, 3H).

### Example 511. Synthesis of 2-chloro-N-[(1R)-1-[3-fluoro-5-(trifluoromethyl)phenyl]ethyl]-6-(morpholine-4-carbonyl)-5H,6H,7H-pyrrolo[3,4-d]pyrimidin-4-amine

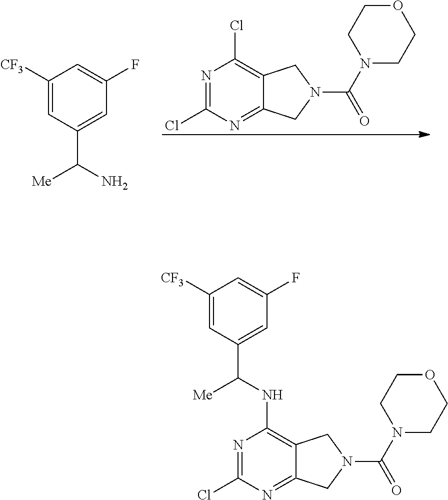 [SMILES: CC(N)c1cc(F)cc(C(F)(F)F)c1 ; O=C(N1CCOCC1)N1Cc2nc(Cl)nc(Cl)c2C1 ; CC(Nc1nc(Cl)nc2c1CN(C(=O)N1CCOCC1)C2)c1cc(F)cc(C(F)(F)F)c1]

### Step 1.

2-Chloro-N-[(1R)-1-[3-fluoro-5-(trifluoromethyl)phenyl]ethyl]-6-(morpholine-4-carbonyl)-5H,6H,7H-pyrrolo[3,4-d]pyrimidin-4-amine was synthesized in a manner similar to 2-chloro-N-{1-[3-(difluoromethyl)-2-fluorophenyl]ethyl}-6-(morpholine-4-carbonyl)-5H,6H,7H-pyrrolo[3,4-d]pyrimidin-4-amine except 1-[3-(difluoromethyl)-2-fluorophenyl]ethan-1-amine was substituted with (1R)-1-[3-fluoro-5-(trifluoromethyl)phenyl]ethan-1-amine to give (227 mg, 80% yield). LCMS (ESI): m/z: [M−H] calculated for C₂₀H₂₀ClF₄N₅O₂: 473.12. found 471.74; ¹H NMR (300 MHz, DMSO-d₆) δ ppm 8.16 (d, J=7.6 Hz, 1H), 7.64 (s, 1H), 7.60-7.52 (m, 2H), 5.42-5.26 (m, 1H), 4.67-4.45 (m, 4H), 3.70-3.55 (m, 4H), 3.29-3.19 (m, 4H), 1.50 (d, J=7.0 Hz, 3H).

### Example 512. Synthesis of 2-chloro-N-[(1R)-1-[3-chloro-5-(trifluoromethyl)phenyl]ethyl]-6-(morpholine-4-carbonyl)-5H,6H,7H-pyrrolo[3,4-d]pyrimidin-4-amine

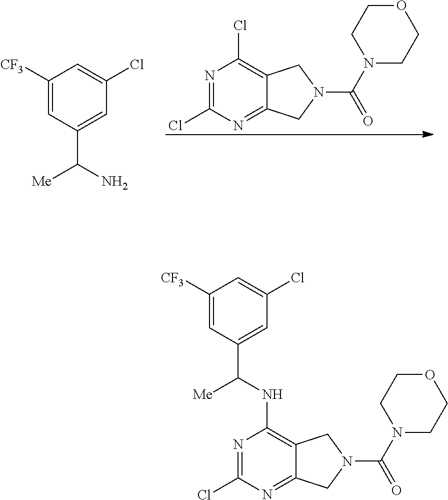 [SMILES: CC(N)c1cc(Cl)cc(C(F)(F)F)c1 ; O=C(N1CCOCC1)N1Cc2nc(Cl)nc(Cl)c2C1 ; CC(Nc1nc(Cl)nc2c1CN(C(=O)N1CCOCC1)C2)c1cc(Cl)cc(C(F)(F)F)c1]

### Step 1.

2-Chloro-N-[(1R)-1-[3-chloro-5-(trifluoromethyl)phenyl]ethyl]-6-(morpholine-4-carbonyl)-5H,6H,7H-pyrrolo[3,4-d]pyrimidin-4-amine was synthesized in a manner similar to 2-chloro-N-{1-[3-(difluoromethyl)-2-fluorophenyl]ethyl}-6-(morpholine-4-carbonyl)-5H,6H,7H-pyrrolo[3,4-d]pyrimidin-4-amine except 1-[3-(difluoromethyl)-2-fluorophenyl]ethan-1-amine was substituted with (1R)-1-[3-chloro-5-(trifluoromethyl)phenyl]ethan-1-amine to give (251 mg, 87% yield). LCMS (ESI): m/z: [M−H] calculated for C₂₀H₂₀Cl₂F₃N₅O₂: 489.09. found 487.65; H NMR (300 MHz, DMSO-d₆) δ ppm 8.16 (d, J=7.6 Hz, 1H), 7.78 (s, 1H), 7.77-7.72 (m, 2H), 5.38-5.27 (m, 1H), 4.68-4.45 (m, 4H), 3.67-3.58 (m, 4H), 3.29-3.19 (m, 4H), 1.50 (d, J=7.0 Hz, 3H).

### Example 513. Synthesis of 2-chloro-N-[(1R)-1-[3-bromo-5-(trifluoromethyl)phenyl]ethyl]-6-(morpholine-4-carbonyl)-5H,6H,7H-pyrrolo[3,4-d]pyrimidin-4-amine

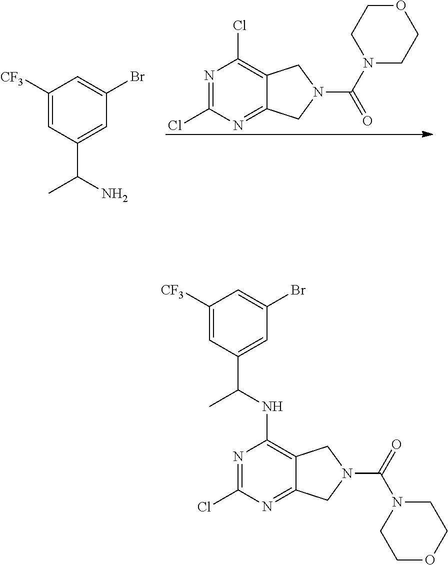 [SMILES: CC(N)c1cc(Br)cc(C(F)(F)F)c1 ; O=C(N1CCOCC1)N1Cc2nc(Cl)nc(Cl)c2C1 ; CC(Nc1nc(Cl)nc2c1CN(C(=O)N1CCOCC1)C2)c1cc(Br)cc(C(F)(F)F)c1]

2-Chloro-N-[(1R)-1-[3-bromo-5-(trifluoromethyl)phenyl]ethyl]-6-(morpholine-4-carbonyl)-5H,6H,7H-pyrrolo[3,4-d]pyrimidin-4-amine was synthesized in a manner similar to 2-chloro-N-{1-[3-(difluoromethyl)-2-fluorophenyl]ethyl}-6-(morpholine-4-carbonyl)-5H,6H,7H-pyrrolo[3,4-d]pyrimidin-4-amine except 1-[3-(difluoromethyl)-2-fluorophenyl]ethan-1-amine was substituted with (1R)-1-[3-bromo-5-(trifluoromethyl)phenyl]ethan-1-amine to give (160 mg, 60% yield). LCMS (ESI): m/z: [M+H] calculated for C₂₀H₂₀BrClF₃N₅O₂: 533.04. found 535.9; ¹H NMR (300 MHz, DMSO-d₆) δ ppm 8.15 (d, J=7.6 Hz, 1H), 7.92 (s, 1H), 7.85 (s, 1H), 7.79 (s, 1H), 5.32 (p, J=6.4 Hz, 1H), 4.67-4.39 (m, 4H), 3.62 (t, J=4.6 Hz, 4H), 3.24 (t, J=4.7 Hz, 4H), 1.50 (d, J=7.0 Hz, 3H).

### Example 514. Synthesis of N-[(1R)-1-[3-amino-5-(trifluoromethyl)phenyl]ethyl]-2′-chloro-7′,8′-dihydro-5′H-spiro[1,3-dioxolane-2,6′-quinazoline]-4′-amine

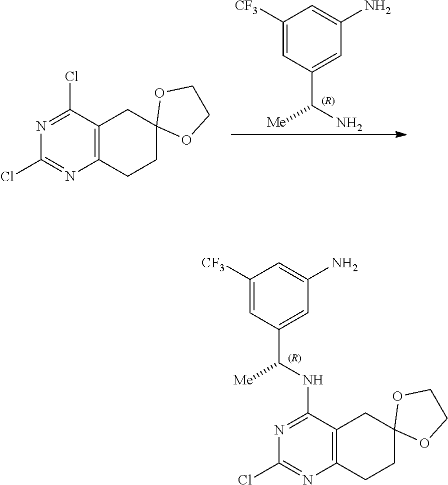

### Step 1.

A mixture of 2′,4′-dichloro-7′,8′-dihydro-5′H-spiro[1,3-dioxolane-2,6′-quinazoline] (104 mg, 0.4 mmol) and 3-(1-aminoethyl)-5-(trifluoromethyl)aniline HCl salt (101 mg, 0.42 mmol) in DMSO (3 mL) was purged with Ar. DIPEA (0.28 mL, 1.6 mmol) was added and the mixture was heated to 150° C. under microwave irradiation and stirred for 1 h. The mixture was diluted with H₂O and Et₂O and the aqueous layer was extracted with Et₂O (×2) and Et₂O/EtOAc (1:1). The combined organic layers were washed with H₂O, dried over Na₂SO₄ and filtered. The filtrate was concentrated under reduced pressure and the crude residue was purified by preparative HPLC to give N-[(1R)-1-[3-amino-5-(trifluoromethyl)phenyl]ethyl]-2′-chloro-7′,8′-dihydro-5′H-spiro[1,3-dioxolane-2,6′-quinazoline]-4′-amine (102 mg, 60% yield). LCMS (ESI): m/z: [M+H] calculated for C₁₉H₂₀ClF₃N₄O₂: 428.12. found 429.01; ¹H NMR (300 MHz, DMSO-d₆) δ ppm 7.38 (d, J=8.0 Hz, 1H), 6.85 (s, 1H), 6.77 (s, 1H), 6.70 (s, 1H), 5.56 (s, 2H), 5.20 (p, J=7.1 Hz, 1H), 3.98 (s, 4H), 2.69 (t, J=6.7 Hz, 2H), 2.62 (s, 2H), 1.88 (t, J=6.7 Hz, 2H), 1.45 (d, J=7.0 Hz, 3H).

### Example 515. Synthesis of 2-chloro-N-[(1R)-1-[3-methyl-5-(trifluoromethyl)phenyl]ethyl]-6-(morpholine-4-carbonyl)-5H,6H,7H-pyrrolo[3,4-d]pyrimidin-4-amine

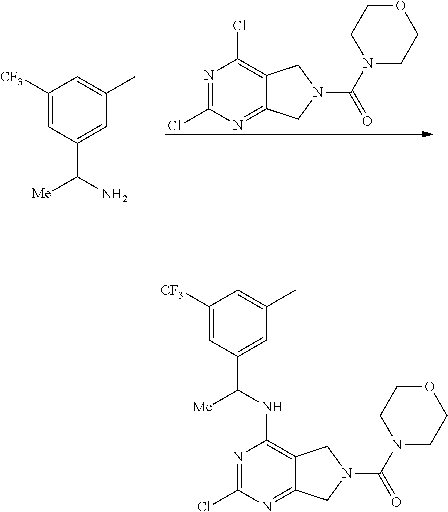 [SMILES: Cc1cc(C(C)N)cc(C(F)(F)F)c1 ; O=C(N1CCOCC1)N1Cc2nc(Cl)nc(Cl)c2C1 ; Cc1cc(C(C)Nc2nc(Cl)nc3c2CN(C(=O)N2CCOCC2)C3)cc(C(F)(F)F)c1]

### Step 1.

2-Chloro-N-[(1R)-1-[3-methyl-5-(trifluoromethyl)phenyl]ethyl]-6-(morpholine-4-carbonyl)-5H,6H,7H-pyrrolo[3,4-d]pyrimidin-4-amine was synthesized in a manner similar to 2-chloro-N-{1-[3-(difluoromethyl)-2-fluorophenyl]ethyl}-6-(morpholine-4-carbonyl)-5H,6H,7H-pyrrolo[3,4-d]pyrimidin-4-amine except 1-[3-(difluoromethyl)-2-fluorophenyl]ethan-1-amine was substituted with (1R)-1-[3-methyl-5-(trifluoromethyl)phenyl]ethan-1-amine HCl salt to give (148 mg, 64% yield). LCMS (ESI): m/z: [M−H] calculated for C₂₁H₂₃ClF₃N₅O₂: 469.15. found 467.76; ¹H NMR (300 MHz, DMSO-d₆) δ ppm 8.15 (d, J=7.8 Hz, 1H), 7.54 (s, 1H), 7.50 (s, 1H), 7.42 (s, 1H), 5.35-5.26 (m, 1H), 4.66-4.43 (m, 4H), 3.67-3.58 (m, 4H), 3.27-3.20 (m, 4H), 2.38 (s, 3H), 1.49 (d, J=7.0 Hz, 3H).

### Example 516. Synthesis of [4-[1-[3-(1-hydroxyethyl)phenyl]ethylamino]-5,7-dihydropyrrolol3,4-d1pyrimidin-6-yl-morpholino-methanone

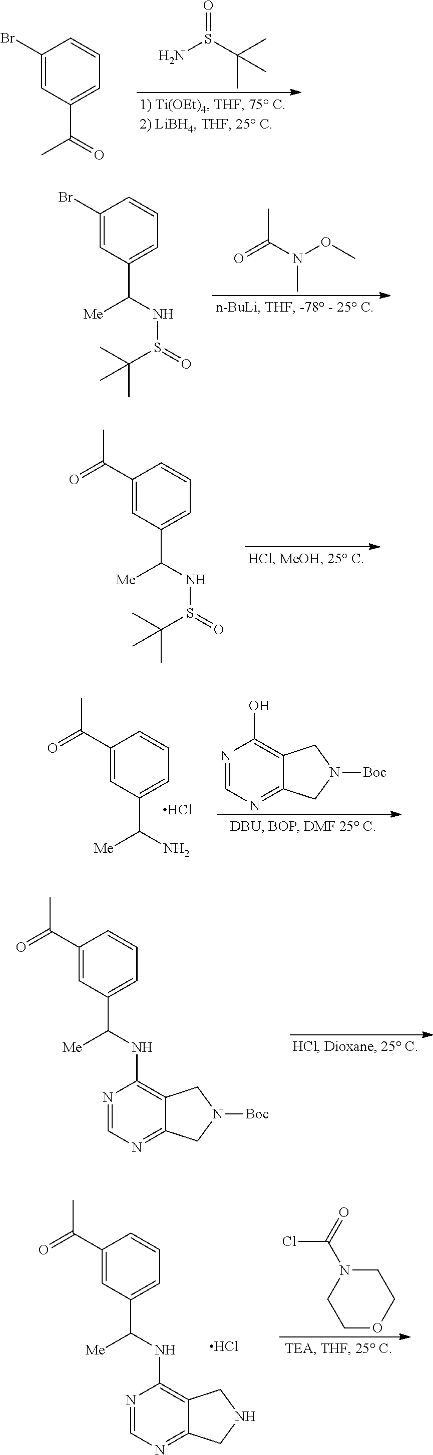

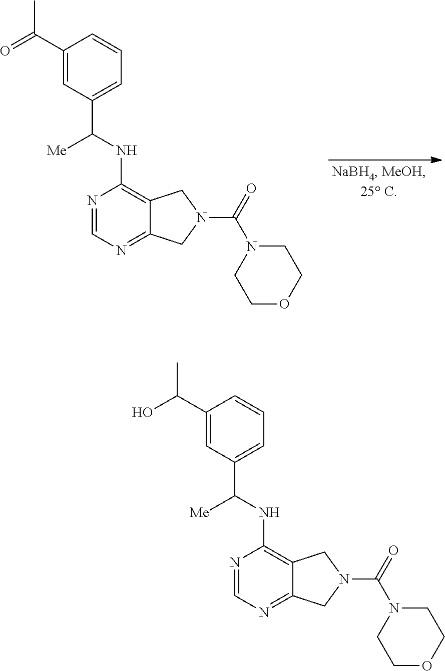 [SMILES: CC(=O)c1cccc(C(C)Nc2ncnc3c2CN(C(=O)N2CCOCC2)C3)c1 ; CC(O)c1cccc(C(C)Nc2ncnc3c2CN(C(=O)N2CCOCC2)C3)c1]

### Step 1.

To a mixture of 1-(3-bromophenyl)ethanone (3.31 mL, 25.1 mmol) and 2-methylpropane-2-sulfinamide (3.04 g, 25.1 mmol) in THF (50 mL), Ti(OEt)₄ (10.4 mL, 50.2 mmol) was added in one portion under N₂. The mixture was heated to 75° C. and stirred for 14 h. The mixture was cooled to 0° C., MeOH (937 μL, 23.1 mmol) and LiBH₄ (504 mg, 23.2 mmol) were then added, and the resulting mixture was stirred for 2 h at 25° C. H₂O (30 mL) was added and the mixture was extracted with EtOAc. The combined organic extracts were treated with brine, dried with Na₂SO₄, filtered, and concentrated under reduced pressure to afford N-[1-(3-bromophenyl)ethyl]-2-methyl-propane-2-sulfinamide (5.5 g, 78% yield). LCMS (ESI): m/z: [M+H] calculated for C₁₂H₁₉BrNOS: 304.0. found: 304.0.

### Step 2.

To a mixture of N-[1-(3-bromophenyl)ethyl]-2-methyl-propane-2-sulfinamide (2.0 g, 6.57 mmol) in THF (35 mL) was added a 2.5 M solution of n-Buli (7.89 mL, 19.7 mmol) in one portion at −78° C. under N₂. The mixture was stirred at −78° C. for 30 min, then N-methoxy-N-methyl-acetamide (908 μL, 8.55 mmol) in THF (5 mL) was added and the mixture was stirred at 25° C. 2 h. The mixture was added to ice water (50 mL), stirred for 20 min and extracted with EtOAc. The combined organic extracts were washed with brine, dried with Na₂SO₄, filtered, and concentrated under reduced pressure to give N-[1-(3-acetylphenyl)ethyl]-2-methyl-propane-2-sulfinamide (800 mg, crude), which was taken directly to the next reaction.

### Step 3.

N-[1-(3-acetylphenyl)ethyl]-2-methyl-propane-2-sulfinamide (794 mg, 2.97 mmol) was dissolved in a 4M solution of HCl in methanol (10 mL, 40 mmol) and the mixture was stirred at 25° C. for 30 min. The mixture was then filtered and concentrated under reduced pressure to give 1-[3-(1-aminoethyl)phenyl]ethanone hydrochloride (450 mg, 42% yield over 2 steps). ¹H NMR (400 MHz, DMSO-d6) δ ppm 8.64 (br s, 3H) 8.16 (s, 1H) 7.95 (d, J=7.6 Hz, 1H) 7.79 (d, J=7.6 Hz, 1H) 7.58 (t, J=7.6 Hz, 1H) 4.48-4.52 (m, 1H) 2.61 (s, 3H) 1.54 (d, J=6.4 Hz, 3H).

### Step 4.

To a mixture of 1-[3-(1-aminoethyl)phenyl]ethanone hydrochloride (250 mg, 1.53 mmol) and tert-butyl 4-oxo-5,7-dihydro-4aH-pyrrolo[3,4-d]pyrimidine-6-carboxylate (363 mg, 1.53 mmol) in DMF (12 mL) were added DBU (693 μL, 4.60 mmol) and BOP (1.02 g, 2.30 mmol) in one portion under N₂. The mixture was stirred at 25° C. for 14 h. The mixture was then filtered and concentrated under reduced pressure. The crude residue was purified by flash column chromatography to afford tert-butyl 4-[[(1R)-1-(3-acetylphenyl)ethyl]amino]-5,7-dihydropyrrolo[3,4-d]pyrimidine-6-carboxylate (200 mg, 34% yield). LCMS (ESI): m/z: [M+H] calculated for C₂₁H₂₇N₄O₃: 383.2. found: 383.2.

### Step 5.

tert-Butyl 4-[1-(3-acetylphenyl) ethylamino]-5,7-dihydropyrrolo[3,4-d]pyrimidine-6-carboxylate (170 mg, 445 μmol) was dissolved in a 4M solution of HCl in dioxane (10 mL, 40 mmol) and was stirred at 25° C. for 30 min. The mixture was filtered and concentrated under reduced pressure to give 1-[3-[1-(6,7-dihydro-SH-pyrrolo[3,4-d]pyrimidin-4-ylamino)ethyl]phenyl]ethenone hydrochloride (75 mg, crude). LCMS (ESI): m/z: [M+H] calculated for C₁₋₆H₁₉N₄O: 283.2. found: 283.1.

### Step 6.

To a mixture of morpholine-4-carbonyl chloride (40.7 mg, 273 μmol) and TEA (173 μL, 1.24 mmol) in THF (2 mL) was added 1-[3-[1-(6,7-dihydro-5H-pyrrolo[3,4-d]pyrimidin-4-ylamino)ethyl]phenyl]ethanone hydrochloride (70 mg, 220 μmol). The mixture was stirred at 25° C. for 2 h. The mixture was filtered and concentrated under reduced pressure. The residue was purified by prep-TLC to afford 1-[3-[1-[[6-(morpholine-4-carbonyl)-5,7-dihydropyrrolo[3,4-d]pyrimidin-4-yl]amino]ethyl]phenyl]ethanone (50 mg, 51% yield). LCMS (ESI): m/z: [M+H] calculated for C₂₁H₂₆N₅O₃: 396.2. found: 396.2; H NMR (400 MHz, DMSO-d₆) δ ppm 8.30 (br s, 1H) 7.96 (s, 1H) 7.82 (d, J=7.6 Hz, 1H) 7.72 (d, J=7.6 Hz, 1H) 7.64 (d, J=7.6 Hz, 1H) 7.46 (t, J=7.6 Hz, 1H) 5.41 (t, J=7.2 Hz, 1H) 4.47-4.69 (m, 4H) 3.60-3.66 (m, 4H) 3.26 (m, 4H) 2.57 (s, 3H) 1.50 (d, J=6.8 Hz, 3H).

### Step 7.

To a mixture of 1-[3-[1-[[6-(morpholine-4-carbonyl)-5,7-dihydropyrrolo[3,4-d]pyrimidin-4-yl]amino]ethyl]phenyl]ethanone (45 mg, 114 μmol) in MeOH (1 mL) was added NaBH₄ (4.3 mg, 114 μmol) 0° C. under N₂. The mixture was warmed to 25° C. and stirred for 2 h. The mixture was quenched with water (0.2 mL), filtered, and concentrated under reduced pressure. The crude residue was purified by prep-HPLC to afford [4-[1-[3-(1-hydroxyethyl)phenyl]ethylamino]-5,7-dihydropyrrolo[3,4-d]pyrimidin-6-yl]-morpholino-methanone (10 mg, 22% yield). LCMS (ESI): m/z: [M+H] calculated for C₂₁H₂₈N5O₃: 398.2. found: 398.1; ¹H NMR (400 MHz, CHLOROFORM-d) δ ppm 8.52 (s, 1H) 7.40 (s, 1H) 7.38-7.25 (m, 3H), 5.46-5.36 (m, 1H) 4.98-4.90 (m, 1H) 4.73 (br s, 1H) 3.74 (t, J=4.8 Hz, 4H) 3.36 (t, J=4.8 Hz, 4H) 1.94 (d, J=14.4 Hz, 1H) 1.62 (d, J=6.8 Hz, 3H) 1.51 (dd, J=6.8, 2.0 Hz, 3H).

### Example 517. Synthesis of [2-chloro-4-[1-[3-(3-fluoroazetidin-3-yl)phenyl]ethylamino]-5,7-dihydropyrrolo[3,4-d]pyrimidin-6-yl]-morpholino-methanone

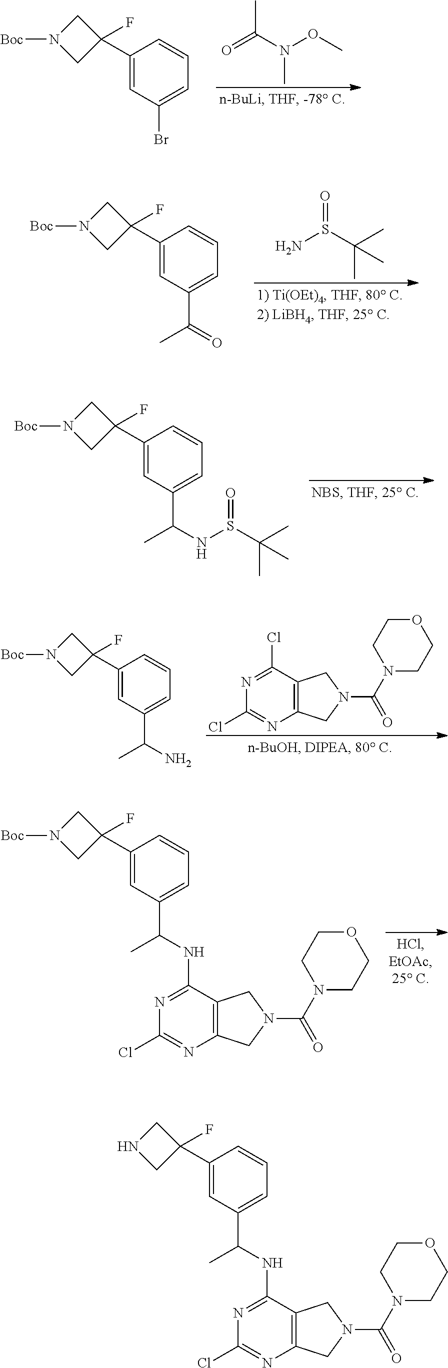

### Step 1.

To a solution of tert-butyl 3-(3-bromophenyl)-3-fluoro-azetidine-1-carboxylate (1.0 g, 3.03 mmol) in THF (10 ml) was added a 2.5M solution of n-BuLi (3.03 ml, 7.58 mmol) at −78° C. The mixture was stirred for 1 h, then N-methoxy-N-methyl-acetamide (468 mg, 4.54 mmol, 482.94 μL) was added to the reaction at −78° C. and the mixture was stirred for 4 h. The mixture was quenched with water, extracted with EtOAc, treated with brine, dried over Na₂SO₄, filtered, and concentrated under reduced pressure to give a residue. The crude residue was purified by silica gel chromatography to give tert-butyl 3-(3-acetylphenyl)-3-fluoro-azetidine-1-carboxylate (240 mg, 9% yield). ¹H NMR (400 MHz, CHLOROFORM-d) δ ppm 7.98 (s, 1H) 7.88 (d, J=7.6 Hz, 1H) 7.61 (d, J=7.6 Hz, 1H) 7.47 (t, J=8.0 Hz, 1H) 4.36 (dd, J=22.4, 10.0 Hz, 2H) 4.20 (dd, J=19.2, 10.0 Hz, 2H) 2.56 (s, 3H) 1.40 (s, 9H).

### Step 2.

To a mixture of tert-butyl 3-(3-acetylphenyl)-3-fluoro-azetidine-1-carboxylate (240 mg, 818 μmol) and 2-methylpropane-2-sulfinamide (198 mg, 1.64 mmol) in THF (2.5 mL), Ti(OEt)₄ (679 μL, 3.27 mmol) was added. The mixture was heated to 80° C. and stirred for 4 h. The mixture was cooled to 0° C., then a 2M solution of LiBH₄ (409 μL, 818 μmol) was added dropwise and the resulting mixture was stirred for 1 h at 0° C. Water was added to the reaction mixture, which was then extracted with EtOAc, treated with brine, dried over Na₂SO₄, filtered, and concentrated under reduced pressure. The crude residue was purified by flash column chromatography to give tert-butyl 3-[3-[1-(tert-butylsulfinylamino)ethyl]phenyl]-3-fluoro-azetidine-1-carboxylate (120 mg, 37% yield). LCMS (ESI): m/z: [M+H] calculated for C₂₀H₃₂FN₂O₃S: 399.2. found 399.2.

### Step 3.

A solution of tert-butyl 3-[3-[1-(tert-butylsulfinylamino)ethyl]phenyl]-3-fluoro-azetidine-1-carboxylate (120 mg, 301 μmol) and N-Bromosuccinimide (59.0 mg, 331 μmol) in THF (1.5 ml) was stirred at 25° C. for 1 h. The mixture was concentrated under reduced pressure to give a residue, which was purified by prep-HPLC to give tert-butyl 3-[3-(1-aminoethyl)phenyl]-3-fluoro-azetidine-1-carboxylate (60 mg, 68% yield). LCMS (ESI): m/z: [M+H] calculated for C₁₆H₂₄FN₂O₂: 295.2. found 295.1.

### Step 4.

To a solution of tert-butyl 3-[3-(1-aminoethyl)phenyl]-3-fluoro-azetidine-1-carboxylate (60 mg, 204 μmol) in n-BuOH (1.5 mL) were added 2,4-dichloro-5,7-dihydropyrrolo [3,4-d]pyrimidin-6-yl)-morpholino-methanone (61.8 mg, 204 μmol) and DIPEA (142 μL, 815 μmol). The mixture was stirred for 3 h at 80° C. The mixture was quenched with water, extracted with EtOAc, treated with brine, dried over Na₂SO₄, filtered, and concentrated under reduced pressure to give a residue. The crude residue was purified by column chromatography to give tert-butyl 3-[3-[1-[[2-chloro-6-(morpholine-4-carbonyl)-5,7-dihydropyrrolo[3,4-d]pyrimidin-4-yl]amino]ethyl]phenyl]-3-fluoro-azetidine-1-carboxylate (100 mg, 87% yield). LCMS (ESI): m/z: [M+H] calculated for C₂₇H₃₅ClFN₆O₄: 561.2. found 561.3.

### Step 5.

tert-butyl 3-[3-[1-[[2-chloro-6-(morpholine-4-carbonyl)-5,7-dihydropyrrolo [3,4-d]pyrimidin-4-yl]amino]ethyl]phenyl]-3-fluoro-azetidine-1-carboxylate (100 mg, 178 μmol) was dissolved in a 4M solution of HCl in EtOAc (89.1 μL, 356 μmol) and the solution was stirred for 2 h at 25° C. The mixture was concentrated under reduced pressure to give a residue. The crude residue was purified by prep-HPLC to give [2-chloro-4-[1-[3-(3-fluoroazetidin-3-yl)phenyl]ethylamino]-5,7-dihydropyrrolo[3,4-d]pyrimidin-6-yl]-morpholino-methanone (40 mg, 45% yield). LCMS (ESI): m/z: [M+H] calculated for C₂₂H₂₇ClFN₆O₂: 461.2. found 461.1; ¹H NMR (400 MHz, CHLOROFORM-d) δ ppm 7.90 (s, 1H) 7.47-7.38 (m, 3H) 6.45 (br s, 1H) 5.52 (br s, 1H) 4.34-4.34 (m, 8H) 3.77-3.66 (m, 4H) 3.37-3.28 (m, 4H) 1.61 (d, J=6.8 Hz, 3H).

### Example 518. Synthesis of [2-chloro-4-[[(1R)-1-[3-(3-hydroxyazetidin-1-yl)phenyl]ethyl]amino]-5,7-dihydropyrrolo[3,4-d]pyrimidin-6-yl]-morpholino-methanone

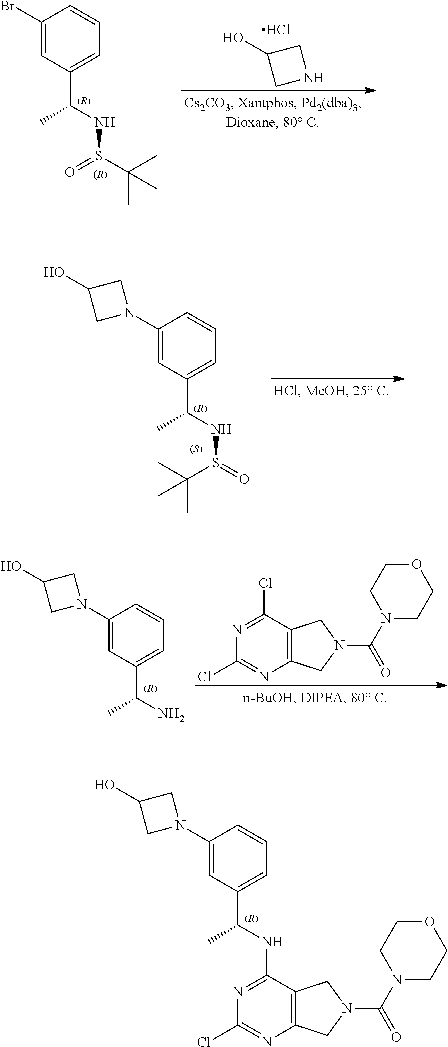

### Step 1.

To a mixture of N-[(1R)-1-(3-bromophenyl)ethyl]-2-methyl-propane-2-sulfinamide (0.50 g, 1.64 mmol), azetidin-3-ol hydrochloride (360 mg, 3.29 mmol) and Cs₂CO₃ (1.61 g, 4.93 mmol) in dioxane (5 mL) were added Xantphos (95.1 mg, 164 μmol) and Pd₂(dba)₃ (75.3 mg, 82.2 μmol) under N₂. The mixture was heated to 100° C. and was stirred for 18 h under N₂. The reaction mixture was filtered and the filtrate was concentrated under reduced pressure to give a residue, which was purified by flash column chromatography to give N-[(1R)-1-[3-(3-hydroxyazetidin-1-yl)phenyl]ethyl]-2-methyl-propane-2-sulfinamide (160 mg, 33% yield). LCMS (ESI): m/z: [M+H] calculated for C₁₅H₂₅N₂O₂S: 297.2. found 297.3.

### Step 2.

To a mixture of N-[(1R)-1-[3-(3-hydroxyazetidin-1-yl)phenyl]ethyl]-2-methyl-propane-2-sulfonamide (90 mg, 304 μmol) in MeOH (0.9 mL) was added a 4 M solution of HCl in MeOH (152 μL, 608 μmol). The mixture was stirred at 25° C. for 2 h. The reaction mixture was concentrated under reduced pressure, residue was diluted with MeOH (3 mL) and the solution was treated with a solution of NaOH in MeOH until pH=8. The mixture was concentrated under reduced pressure to give a residue, which was then diluted with a 5:1 mixture of CH₂Cl₂: MeOH (3 mL), filtered and concentrated under reduced pressure to give 1-[3-[(1R)-1-aminoethyl]phenyl]azetidin-3-ol hydrochloride (65 mg, 94% yield). LCMS (ESI): m/z: [M+H] calculated for C₁₁H₁₇N₂O: 193.1. found 193.1.

### Step 3.

To a mixture of 1-[3-[(1R)-1-aminoethyl]phenyl]azetidin-3-ol (110 mg, 572 μmol) and (2,4-dichloro-5,7-dihydropyrrolo[3,4-d]pyrimidin-6-yl)-morpholino-methanone (173 mg, 572 μmol) in n-BuOH (2.8 mL) was added DIEA (498 μL, 2.86 mmol). The mixture was stirred at 80° C. for 3 h. The mixture was then filtered and the filtrate was purified by prep-HPLC to give [2-chloro-4-[[(1R)-1-[3-(3-hydroxyazetidin-1-yl)phenyl]ethyl]amino]-5,7-dihydropyrrolo[3,4-d]pyrimidin-6-yl]-morpholino-methanone (14 mg, 5% yield). LCMS (ESI): m/z: [M+H] calculated for C₂₂H₂₈ClN₆O₃: 459.2. found: 459.3; ¹H NMR (400 MHz, METHANOL-d₄) δ ppm 7.14 (t, J=8.0 Hz, 1H), 6.76 (d, J=7.6 Hz, 1H), 6.55 (s, 1H), 6.39 (d, J=8.0 Hz, 1H), 5.37-5.30 (m, 1H), 4.69-4.61 (m, 1H), 4.61-4.53 (m, 4H), 4.12 (t, J=8.0 Hz, 2H), 3.75-3.68 (m, 4H), 3.60-3.53 (m, 2H), 3.39-3.33 (m, 4H), 1.54 (d, J=7.2 Hz, 3H).

### Example 519. Synthesis of [2-chloro-4-[[(1R)-1-[3-(1-fluoro-2-hydroxy-ethyl)phenyl]ethyl]amino]-5,7-dihydropyrrolo[3,4-d]pyrimidin-6-yl]-morpholino-methanone

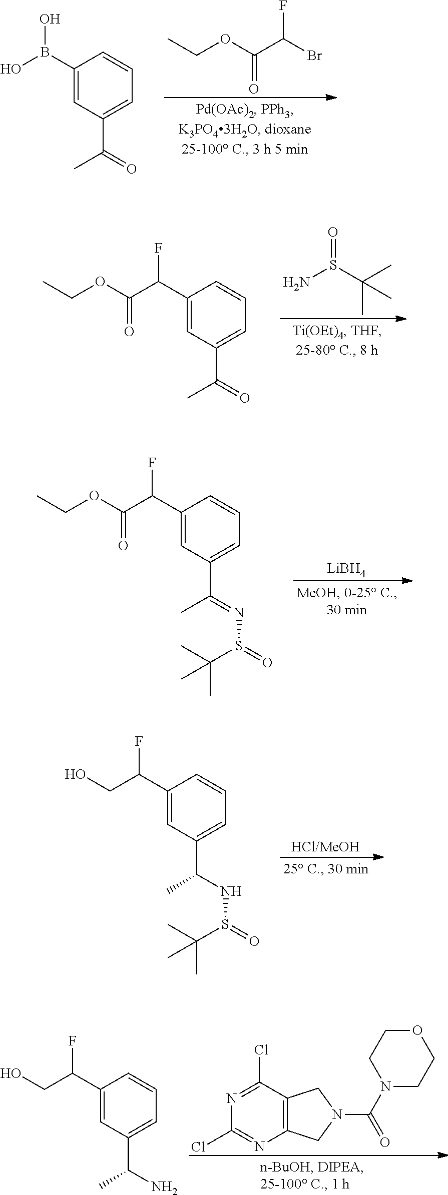

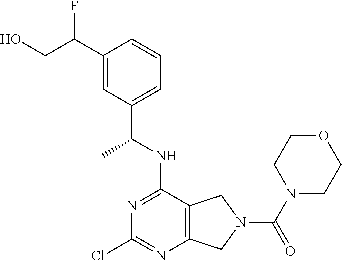 [SMILES: C[C@@H](Nc1nc(Cl)nc2c1CN(C(=O)N1CCOCC1)C2)c1cccc(C(F)CO)c1]

### Step 1.

To a mixture of (3-acetylphenyl)boronic acid (885.42 mg, 5.40 mmol, 712.56 μL), K₃PO₄.3H₂O (2.16 g, 8.10 mmol), Pd(OAc)₂ (30.31 mg, 135.00 μmol) and PPh₃ (141.64 mg, 540.00 μmol) was added dioxane (8 mL) in one portion at rt under N₂. The mixture was stirred at rt for 5 min, then ethyl 2-bromo-2-fluoro-acetate (500 mg, 2.70 mmol, 318.47 μL) was added dropwise at rt under N₂. The reaction mixture was stirred and heated to 100° C. for 3 h. The mixture was filtered and concentrated under reduced pressure. The residue was purified by column chromatography to give ethyl 2-(3-acetylphenyl)-2-fluoro-acetate (280 mg, 46.25% yield). LCMS (ESI): m/z: [M+H] calculated for C₁₂H₁₄FO₃: 225.08. found: 225.1; ¹H NMR (400 MHz, DMSO-d6) δ ppm 8.05 (m, 1H) 8.01 (m, 1H) 7.70-7.72 (m, 1H) 7.63-7.65 (m, 1H) 6.22-6.33 (d, 1H) 4.19 (q, J=7.2 Hz, 2H) 2.61 (s, 3H) 1.16 (t, J=7.2 Hz, 3H).

### Step 2.

To a mixture of 2-methylpropane-2-sulfinamide (151.35 mg, 1.25 mmol) and ethyl 2-(3-acetylphenyl)-2-fluoro-acetate (280 mg, 1.25 mmol) in THF (5 mL) was added Ti(OEt)₄ (569.69 mg, 2.50 mmol, 517.90 μL) in one portion at rt under N₂. The mixture was stirred and heated to 80° C. for 8 h to give ethyl 2-[3-[(Z)-N—[(R)-tert-butylsulfinyl]-C-methyl-carbonimidoyl]phenyl]-2-fluoro-acetate (400 mg, 97.84% yield) as a crude mixture, which was used in the next step without purification. LCMS (ESI): m/z: [M+H] calculated for C₁₋₆H₂₃FNO₃S: 328.13. found: 328.1.

### Step 3.

To a mixture of ethyl 2-[3-[(Z)-N-tert-butylsulfinyl-C-methyl-carbonimidoyl]phenyl]-2-fluoro-acetate (400.00 mg, 1.22 mmol) in THF (5 mL) was added MeOH (39.15 mg, 1.22 mmol, 49.44 μL) and LiBH₄ (93.15 mg, 4.28 mmol) in portions at 0° C. under N₂. The mixture was stirred at rt for 30 min. To the mixture was added water (2 mL) and stirred for 5 min. The aqueous phase was extracted with EtOAc. The combined organic phase was washed with brine, dried with anhydrous Na₂SO₄, filtered and concentrated under reduced pressure. The residue was purified by column chromatography to give N-[(1R)-1-[3-(1-fluoro-2-hydroxy-ethyl)phenyl]ethyl]-2-methyl-propane-2-sulfinamide (220 mg, 62.66% yield). LCMS (ESI): m/z: [M+H] calculated for C₁₄H₂₃FNO₂S: 288.14. found: 288.1.

### Step 4.

A mixture of N-[(1R)-1-[3-(1-fluoro-2-hydroxy-ethyl)phenyl]ethyl]-2-methyl-propane-2-sulfinamide (220 mg, 765.50 μmol) in 4M HCl/MeOH (4 mL) was stirred at rt for 30 min. The mixture was concentrated under reduced pressure. To the residue was added sat.NaOH/MeOH solution until the pH=7, then filtered and concentrated under reduced pressure. The crude product was triturated with DCM (1 mL) at rt for 5 min, the filter was concentrated under reduced pressure to give 2-[3-[(1R)-1-aminoethyl]phenyl]-2-fluoro-ethanol (200 mg, crude). LCMS (ESI): m/z: [M+H] calculated for CiOH₁₅FNO: 184.11. found: 184.1.

### Step 5.

To a mixture of (2,4-dichloro-5,7-dihydropyrrolo[3,4-d]pyrimidin-6-yl)-morpholino-methanone (82.73 mg, 272.89 μmol) and 2-[3-[(1R)-1-aminoethyl]phenyl]-2-fluoro-ethanol (100 mg, 545.79 μmol) in n-BuOH (2 mL) was added DIEA (105.81 mg, 818.68 μmol, 142.60 μL) in one portion at rt under N₂. The mixture was stirred and heated to 100° C. for 1 h. The mixture was filtered and concentrated under reduced pressure. The crude product was purified by prep-HPLC to afford [2-chloro-4-[[(1R)-1-[3-(1-fluoro-2-hydroxy-ethyl)phenyl]ethyl]amino]-5,7-dihydropyrrolo[3,4-d]pyrimidin-6-yl]-morpholino-methanone (35 mg, 27.15% yield). LCMS (ESI): m/z: [M+H] calculated for C₂₁H₂₆ClFN₅O₃: 450.16. found: 450.1; ¹H NMR (400 MHz, DMSO-d6) δ ppm 8.11 (br d, J=8.4 Hz, 1H) 7.32-7.41 (m, 3H) 7.24 (d, J=5.2 Hz, 1H) 5.42-5.57 (m, 1H) 5.28-5.30 (m, 1H) 5.17-5.18 (m, 1H) 4.5-4.54 (m, 4H) 3.61-3.72 (m, 6H) 3.23-3.25 (m, 4H) 1.48 (d, J=7.2 Hz, 3H).

### Example 520. Synthesis of 2-chloro-N-[(1R)-1-[3-(cyclopropylamino)phenyl]ethyl]-6-(morpholine-4-carbonyl)-5H,6H,7H-pyrrolo[3,4-d]pyrimidin-4-amine

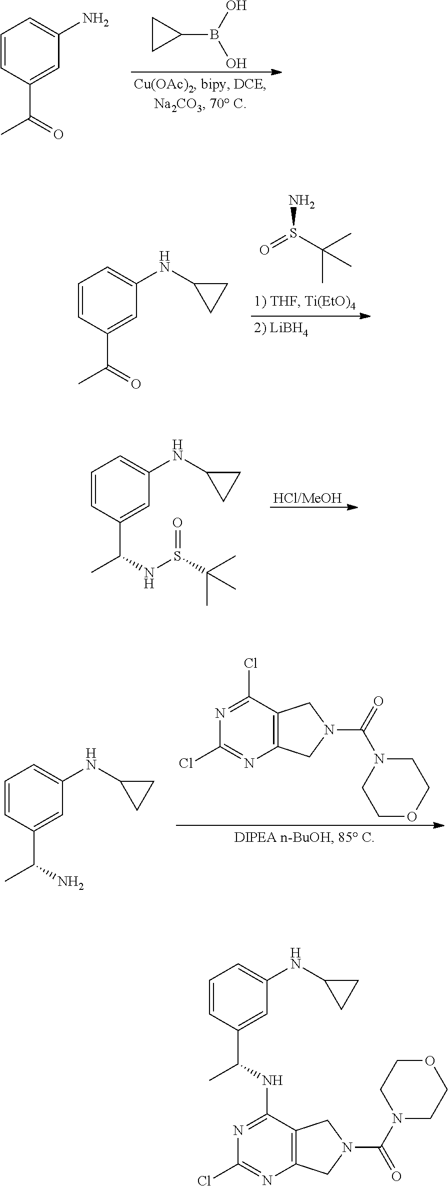

### Step 1.

To a solution of cyclopropylboronic acid (6.36 g, 73.98 mmol) and 1-(3-aminophenyl)ethanone (2 g, 14.80 mmol) in DCE (20 mL) was added bipy (2.31 g, 14.80 mmol), Na₂CO₃ (3.14 g, 29.59 mmol) and Cu(OAc)₂ (2.69 g, 14.80 mmol). The mixture was stirred at 70° C. for 8 h under N₂. After cooling to rt the reaction was filtered and water was added to the filtrate. The aqueous phase was extracted with DCM and the combined organic layers were washed with brine, dried over Na₂SO₄ and the solvent was removed under reduced pressure. The crude residue was purified by silica gel chromatography to give 1-[3-(cyclopropylamino)phenyl]ethanone (750 mg, 28.93% yield). LCMS (ESI): m/z: [M+H] calculated for C₁₁H₁₄NO: 176.10. found 176.1.

### Step 2.

To a solution of 1-[3-(cyclopropylamino)phenyl]ethanone (100 mg, 570.69 μmol) 2-methylpropane-2-sulfinamide (138.34 mg, 1.14 mmol) and Ti(OEt)₄ (520.72 mg, 2.28 mmol, 473.38 μL) in THF (1 mL) was stirred at 80° C. for 4 h. After cooling to 0° C., LiBH₄ (2 M, 285.34 μL) was added and the resulting mixture was stirred for 1 h at 0° C. Water was added and the reaction was filtered. The aqueous phase was extracted with EtOAc and the combined organic phases were washed with brine, dried over anhydrous Na₂SO₄ and the solvent was removed under reduced pressure. The crude residue was purified by prep-TLC to give N-[(1R)-1-[3-(cyclopropylamino)phenyl]ethyl]-2-methyl-propane-2-sulfinamide (60 mg, 37.49% yield).

### Step 3.

A solution of N-[(1R)-1-[3-(cyclopropylamino)phenyl]ethyl]-2-methyl-propane-2-sulfinamide (60 mg, 213.96σμoλ) in HCl/MeOH (4 M, 1 mL) was stirred at 25° C. for 0.5 h. The pH was adjusted to 7 with sat. NaOH/MeOH solution, the solvent was removed under reduced pressure and the crude residue was stirred with DCM/MeOH (5:1, 5 mL) for 10 min. After filtration the solvent was removed under reduced pressure to give 3-[(1R)-1-aminoethyl]-N-cyclopropyl-aniline (44 mg, 86.34% yield). ¹H NMR (400 MHz, CHLOROFORM-d) δ ppm 7.14-7.18 (m, 1H) 6.83 (s, 1H) 6.73-6.78 (m, 2H) 4.16-4.23 (m, 1H) 3.81 (br d, J=0.8 Hz, 2H) 3.49 (s, 1H) 2.37-2.42 (m, 1H) 1.58 (d, J=6.8 Hz, 3H) 0.72-0.75 (m, 2H) 0.48-0.50 (m, 2H).

### Step 4.

To a solution of (2,4-dichloro-5,7-dihydropyrrolo[3,4-d]pyrimidin-6-yl)-morpholino-methanone (68.80 mg, 226.94σμoλ) in n-BuOH (1 mL) was added DIEA (293.30 mg, 2.27 mmol, 395.29 μL) and 3-[(1R)-1-aminoethyl]-N-cyclopropyl-aniline (40 mg, 226.94σμoλ). The mixture was stirred at 85° C. for 10 h. After cooling to rt the solvent was removed under reduced pressure and the crude residue was purified by prep-HPLC to give [2-chloro-4-[[(1R)-1-[3-(cyclopropylamino)phenyl]ethyl]amino]-5,7-dihy dropyrrolo[3,4-d]pyrimidin-6-yl]-morpholino-methanone (12 mg, 11.41% yield). LCMS (ESI): m/z: [M+H] calculated for C₂₂H₂₈ClN₆O₂: 443.19. found 443.3; ¹H NMR (400 MHz, CHLOROFORM-d) δ ppm 7.20 (t, J=7.6 Hz, 1H) 6.72-6.77 (m, 3H) 5.25-5.30 (m, 1H) 4.87 (br s, 1H) 4.56 (s, 4H) 3.71-3.73 (m, 4H) 3.32-3.34 (m, 4H) 2.43-2.45 (m, 1H) 1.59 (d, J=6.8 Hz, 3H) 0.75-0.76 (m, 2H) 0.51-0.53 (m, 2H).

### Example 521. Synthesis of 2-chloro-N-{1-[4-(difluoromethyl)-3-fluoropyridin-2-yl]ethyl}-6-(morpholine-4-carbonyl)-5H,6H,7H-pyrrolo[3,4-d]pyrimidin-4-amine

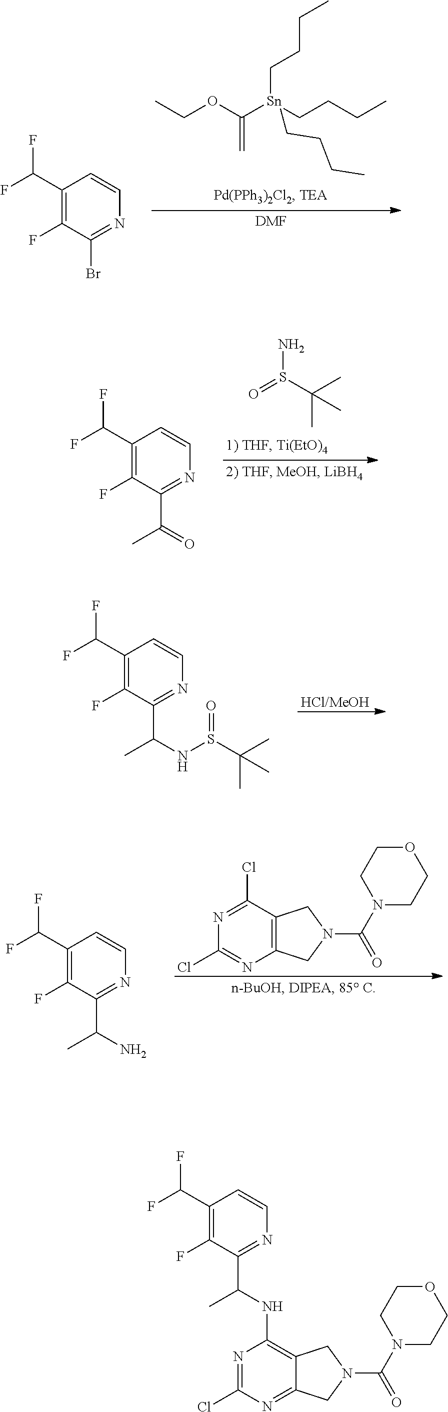

### Step 1.

To a mixture of 2-bromo-4-(difluoromethyl)-3-fluoro-pyridine (0.8 g, 3.54 mmol) in DMF (5 mL) was added Pd(PPh₃)₂Cl₂ (124.23 mg, 177.00σμoλ) and tributyl(1-ethoxyvinyl)stannane (1.53 g, 4.25 mmol, 1.43 mL) in one portion at 25° C. under N₂. The mixture was heated to 100° C. and stirred for 2 h. Aqueous CsF (10 mL) was added and the mixture was stirred for 10 min. After filtration the aqueous phase was extracted with EtOAc and the combined organic phases were washed with brine, dried over Na₂SO₄ and the solvent was removed under reduced pressure. Aqueous HCl (2 M, 8.00 mL) was added and the mixture was stirred at 25° C. for 1 h. Water was added and after extraction with EtOAc the combined organic phases were washed with brine and dried over Na₂SO₄. The solvent was removed under reduced pressure and the crude residue was purified by column chromatography to give 1-[4-(difluoromethyl)-3-fluoro-2-pyridyl]ethanone (0.4 g, 57.42% yield). ¹H NMR (400 MHz, METHANOL-d₄) δ ppm 8.54-8.64 (m, 1H), 7.82-7.84 (t, J=4.4 Hz, 1H), 7.13 (t, J=54.0 Hz, 1H), 2.68 (s, 3H).

### Step 2.

To a mixture of 1-[4-(difluoromethyl)-3-fluoro-2-pyridyl]ethanone (400 mg, 2.11 mmol) in THF (5 mL) was added Ti(OEt)₄ (1.93 g, 8.46 mmol, 1.75 ml) and 2-methylpropane-2-sulfinamide (512.65 mg, 4.23 mmol) at 25° C. under N₂. The mixture was stirred at 80° C. for 2 h. After cooling to 0° C., LiBH₄ (184.28 mg, 8.46 mmol) and MeOH (67.76 mg, 2.11 mmol, 85.58 μL) were added and the reaction was stirred at 0° C. for 1 h. The mixture was poured into ice-water and the aqueous phase was extracted with EtOAc. The combined organic phases were washed with brine and dried over anhydrous Na₂SO₄. The solvent was removed under reduced pressure and the crude residue was purified by column chromatography to give N-[1-[4-(difluoromethyl)-3-fluoro-2-pyridyl]ethyl]-2-methyl-propane-2-sulfonamide (0.470 g, 75.50% yield). LCMS (ESI): m/z: [M+H] calculated for C₁₂H₁₈F₃N₂SO: 294.10. found 295.1.

### Step 3.

To a solution of N-[1-[4-(difluoromethyl)-3-fluoro-2-pyridyl]ethyl]-2-methyl-propane-2-sulfinamide (0.180 g, 611.55υμoλ) in MeOH (7 mL) was added HCl/MeOH (4 M, 305.77 μL) and the reaction was stirred at 25° C. for 0.5 h. The solvent was removed under reduced pressure and the crude residue was dissolved in MeOH (4 mL), and adjusted to pH=7 with sat. NaOH/MeOH solution. The solvent was removed udner reduced pressure and the residue was dissolved in DCM: MeOH (5:1, 5 mL). The mixture was filtered and concentrated to give 1-[4-(difluoromethyl)-3-fluoro-2-pyridyl] ethanamine(50 mg, 42.99% yield). LCMS (ESI): m/z: [M+H] calculated for C₈H₁₀F₃N₂: 191.07. found 191.1.

### Step 4.

To a mixture of 1-[4-(difluoromethyl)-3-fluoro-2-pyridyl]ethanamine (100 mg, 525.86 μmol) and (2,4-dichloro-5,7-dihydropyrrolo[3,4-d]pyrimidin-6-yl)-morpholino-methanone (191.29 mg, 631.03 μmol) in n-BuOH (2 mL) was added DIEA (339.82 mg, 2.63 mmol, 457.98 μL). Then the mixture was heated to 80° C. and stirred for 2 h under N₂. After removing the solvent under reduced pressure the crude residue was purified by prep-HPLC to give [2-chloro-4-[1-[4-(difluoromethyl)-3-fluoro-2-pyridyl]ethylamino]-5,7-dihydropyrrolo[3,4-d]pyrimidin-6-yl]-morpholino-methanone (70 mg, 28.70% yield). LCMS (ESI): m/z: [M+H] calculated for C₁₉H₂₁ClF₃N₆O₄: 456.13. found 457.2; ¹H NMR (400 MHz, METHANOL-d₄) δ ppm 8.46 (d, J=4.8 Hz, 1H), 7.52 (t, J=5.2 Hz, 1H), 7.10 (t, J=54 Hz, 1H), 5.66-5.72 (m, 1H), 4.65 (s, 2H), 4.54 (d, J=1.6 Hz, 2H), 3.71-3.73 (m, 4H), 3.34-3.37 (m, 4H), 1.60 (d, J=7.2 Hz, 3H).

### Example 522. Synthesis of 2-chloro-N-[(1R)-1-[2-chloro-3-(difluoromethyl)phenyl]ethyl]-6-(morpholine-4-carbonyl)-5H,6H,7H-pyrrolo[3,4-d]pyrimidin-4-amine

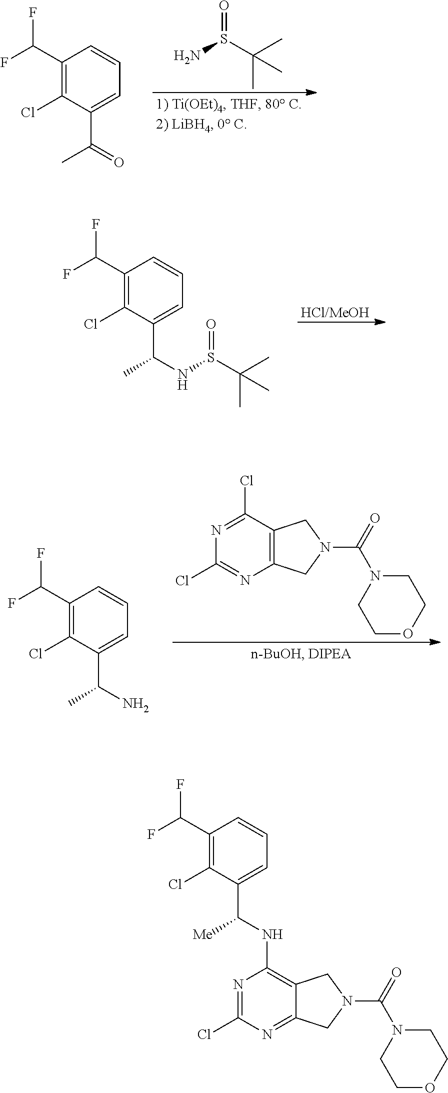

### Step 1.

To a mixture of 1-[2-chloro-3-(difluoromethyl)phenyl]ethanone (250 mg, 1.22 mmol), 2-methylpropane-2-sulfinamide (296.19 mg, 2.44 mmol) and Ti(OEt)₄ (1.11 g, 4.89 mmol, 1.01 mL) in THF (2 mL) was stirred at 80° C. for 4 h. After cooling to 0° C., LiBH₄ (2 M, 610.95 μL) was added and the reaction was stirred for 1 h at 0° C. Water was added and the mixture was extracted with EtOAc. The combined organic phases were washed with brine, dried over Na₂SO₄ and the solvent was removed under reduced pressure. The crude residue was purified by prep-HPLC to get N-[(1R)-1-[2-chloro-3-(difluoromethyl)phenyl]ethyl]-2-methyl-propane-2-sulfinamide (160 mg, 53.33% yield). LCMS (ESI): m/z: [M+H] calculated for C₁₃H₁₉ClF₂NOS: 310.08. found 310.1.

### Step 2.

A solution of N-[(1R)-1-[2-chloro-3-(difluoromethyl)phenyl]ethyl]-2-methyl-propane-2-sulfinamide (150 mg, 484.18 μmol) in HCl/MeOH (1.3 mL, 4 M) was stirred at 25° C. for 0.5 h. The pH was adjusted to 7 with sat. NaOH/MeOH solution. The solvent was removed under reduced pressure and the residue was stirred with DCM/MeOH (10:1) (4 mL) for 10 min and then filtered. The solvent was removed under reduced pressure to give (1R)-1-[2-chloro-3-(difluoromethyl)phenyl]ethanamine (62 mg, 62.27% yield). LCMS (ESI): m/z: [M+H] calculated for C₉H₁₁ClF₂N: 206.05. found 206.1.

### Step 3.

A solution of (1R)-1-[2-chloro-3-(difluoromethyl)phenyl]ethanamine (62.00 mg, 301.51 μmol) in n-BuOH (0.6 mL) was added DIEA (311.74 mg, 2.41 mmol, 420.13 μL) and (2,4-dichloro-5,7-dihydropyrrolo[3,4-d]pyrimidin-6-yl)-morpholino-methanone (100.54 mg, 331.66 μmol). The mixture was stirred at 85° C. for 10 h. After cooling to rt the solvent was removed under reduced pressure and the crude residue was purified by prep-HPLC to give [2-chloro-4-[[(1R)-1-[2-chloro-3-(difluoromethyl)phenyl]ethyl]amino]-5,7-dihydropyrrolo[3,4-d]pyrimidin-6-yl]-morpholino-methanone (61 mg, 42.54% yield). LCMS (ESI): m/z: [M+H] calculated for C₂₀H₂₂Cl₂F₂N₅O₂: 472.10. found 472.1; ¹H NMR (400 MHz, METHANOL-d₄) δ ppm 7.57-7.60 (m, 2H) 7.40-7.44 (m, 1H) 7.11 (t, J=54.8 Hz, 1H) 5.72-5.73 (m, 1H) 4.66 (m, 2H) 4.57 (m, 2H) 3.72-3.74 (m, 4H) 3.35-3.37 (m, 4H) 1.57 (d, J=7.2 Hz, 3H).

### Example 523. Synthesis of 2-chloro-N-[(1R)-1-(4,4-difluoro-3,4-dihydro-1H-2-benzopyran-8-yl)ethyl]-6-(morpholine-4-carbonyl)-5H,6H,7H-pyrrolo[3,4-d]pyrimidin-4-amine

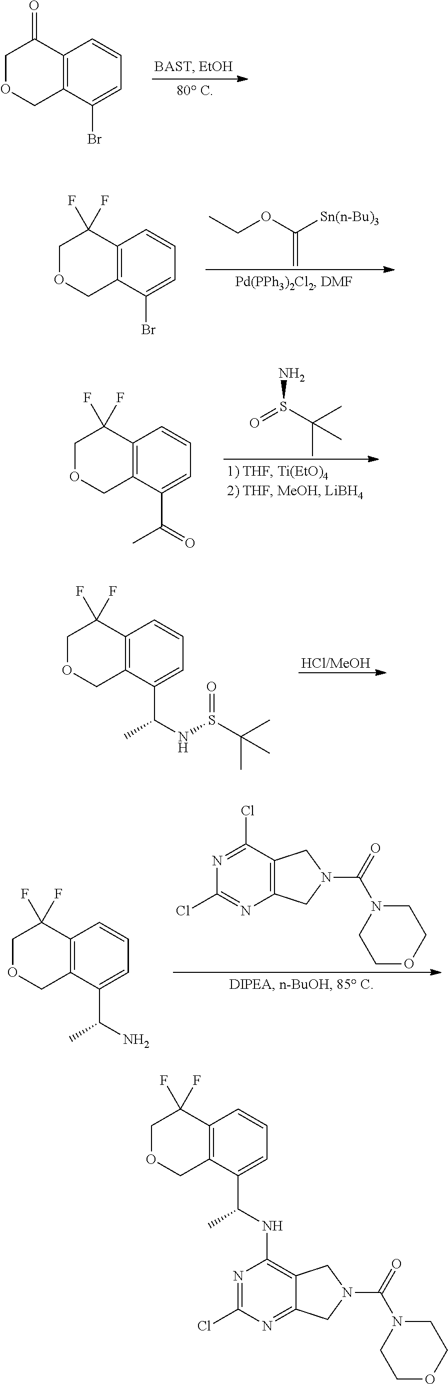

### Step 1.

To a solution of 8-bromoisochroman-4-one (200 mg, 880.85 μmol) in BAST (2 mL) was added EtOH (4.06 mg, 88.08 μmol, 5.15 μL). The mixture was stirred at 80° C. for 2 h. After cooling to rt water was added and the mixture was extracted with EtOAc. Then the combined organic phase was washed with brine and dried with anhydrous Na₂SO₄. The solvent was removed under reduced pressure and the crude residue was purified by column chromatography to give compound 8-bromo-4,4-difluoro-isochromane (400 mg, 91.17% yield). ¹H NMR (400 MHz, METHANOL-d₄) δ ppm 7.69 (d, J=7.6 Hz, 2H), 7.36 (t, J=8.0 Hz, 1H), 4.77 (s, 2H), 4.06-4.11 (m, 2H).

### Step 2.

To a solution of 8-bromo-4,4-difluoro-isochromane (0.4 g, 1.61 mmol) and tributyl(1-ethoxyvinyl)stannane (696.05 mg, 1.93 mmol, 650.51 μL) in DMF (4 mL) was added Pd(PPh₃)₂Cl₂ (56.37 mg, 80.30 μmol). The mixture was stirred at 100° C. for 2 h under N₂. Aqueous CsF (5 mL) was added and the mixture was stirred for 10 min. The aqueous phase was extracted with EtOAc. The combined organic phases were washed with brine, dried over Na₂SO₄ and the solvent was removed under reduced pressure to give 8-(1-ethoxyvinyl)-4,4-difluoro-isochromane (1 g, crude), which was used without further purification. LCMS (ESI): m/z: [M+H] calculated for C₁₃H₁₅F₂O₂: 241.10. found 241.0.

### Step 3.

A solution of 8-(1-ethoxyvinyl)-4,4-difluoro-isochromane (1 g, 4.16 mmol) in 2N HCl (10 mL) was stirred at 25° C. for 1 h. The mixture was extracted with EtOAc and the combined organic phase was washed with brine and dried over Na₂SO₄. The solvent was removed under reduced pressure and the crude residue was purified by column chromatography (SiO₂, Petroleum ether: EtOAc=100:1 to 0:1) to give 1-(4,4-difluoroisochroman-8-yl)ethanone (90 mg, 10.19% yield). LCMS (ESI): m/z: [M+H] calculated for C₁₁H₁₁F₂O₂: 213.1. found 213.2.

### Step 4.

To a mixture of 1-(4,4-difluoroisochroman-8-yl)ethanone (90 mg, 424.14 μmol) and 2-methylpropane-2-sulfinamide (102.81 mg, 848.29 μmol) in THF (1 mL) was added Ti(OEt)₄ (387.00 mg, 1.70 mmol, 351.82 μL) at 25° C. The mixture was stirred at 80° C. for 1 h. After cooling to 0° C., MeOH (13.59 mg, 424.14 μmol, 17.16 μL) and LiBH₄ (36.96 mg, 1.70 mmol) were added to the mixture at 0° C. The mixture was stirred at 25° C. for 1 h. Water was added and the mixture was extracted with EtOAc. The combined organic phases were washed with brine, dried over Na₂SO₄ and the solvent was removed under reduced pressure. The crude residue was purified by column chromatography to give compound N-[(1R)-1-(4,4-difluoroisochroman-8-yl)ethyl]-2-methyl-propane-2-sulfinamide (40 mg, 29.71% yield). LCMS (ESI): m/z: [M+H] calculated for C₁₅H₂₂F₂NO₂S: 318.13. found 318.2;

### Step 5.

To a solution of N-[(1R)-1-(4,4-difluoroisochroman-8-yl)ethyl]-2-methyl-propane-2-sulfinamide (40 mg, 126.03 μmol) in MeOH (2 mL) was added HCl/MeOH (4 M, 63.01 μL). The mixture was stirred at 25° C. for 1 h. The solvent was removed under reduced pressure and the residue was dissolved into MeOH (2 mL). The pH was adjusted to pH=7 with sat. NaOH/MeOH solution. The mixture was then concentrated and redissolved in a mixture of DCM and MeOH (5:1, 5 mL). After filtration the solvent was removed under reduced pressure to give (1R)-1-(4,4-difluoroisochroman-8-yl)ethanamine (40 mg, crude). LCMS (ESI): m/z: [M+H] calculated for C₁₁H₁₄F₂NO: 214.1. found 214.2.

### Step 6.

To a solution of (1R)-1-(4,4-difluoroisochroman-8-yl)ethanamine (40 mg, 187.60 μmol) and (2,4-dichloro-5,7-dihydropyrrolo[3,4-d]pyrimidin-6-yl)-morpholino-methanone (68.24 mg, 225.12 μmol) in n-BuOH (1 mL) was added DIEA (242.46 mg, 1.88 mmol, 326.76 μL). The mixture was stirred at 85° C. for 1 h. After cooling to rt the solvent was removed under reduced pressure and the crude residue was purified by prep-HPLC to give [2-chloro-4-[[(1R)-1-(4,4-difluoroisochroman-8-yl)ethyl]amino]-5,7-dihydropyrrolo[3,4-d]pyrimidin-6-yl]-morpholino-methanone (23 mg, 25.16% yield). LCMS (ESI): m/z: [M+H] calculated for C₂₂H₂₅ClF₂N₅O₃: 480.15. found 480.2; ¹H NMR (400 MHz, METHANOL-d₄) δ ppm 7.53-7.58 (m, 1H), 7.37-7.42 (m, 1H), 5.36 (d, J=15.2 Hz, 1H), 5.15-5.20 (m, 1H), 4.97 (d, J=15.6 Hz, 1H), 4.54-4.61 (m, 4H), 4.03-4.12 (m, 2H), 3.70-3.73 (m, 4H), 3.33-3.36 (m, 4H), 1.54 (d, J=6.8 Hz, 3H).

### Example 524. Synthesis of 2-chloro-N-[(1R)-1-[3-(cyclopropoxydifluoromethyl)phenyl]ethyl]-6-(morpholine-4-carbonyl)-5H,6H,7H-pyrrolo[3,4-d]pyrimidin-4-amine

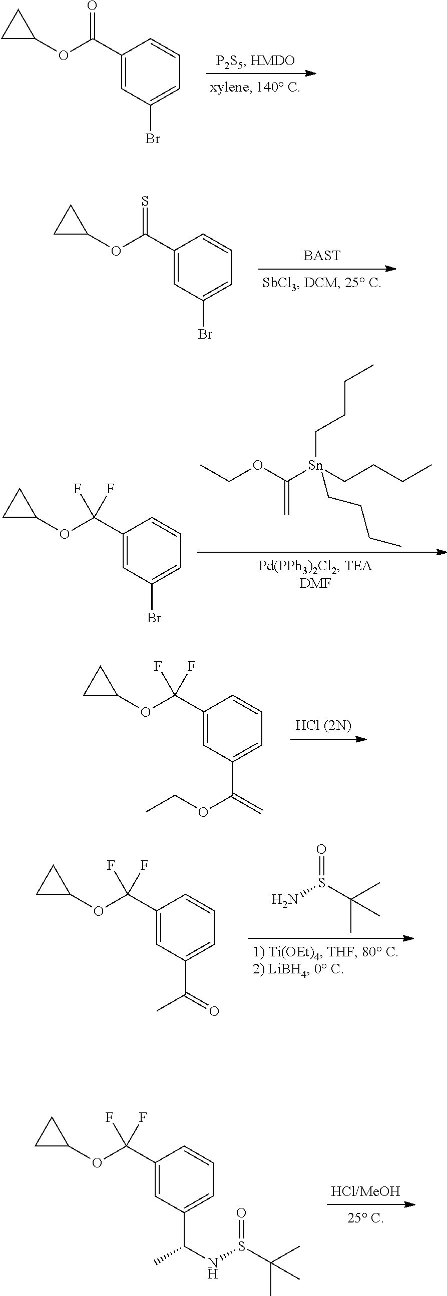

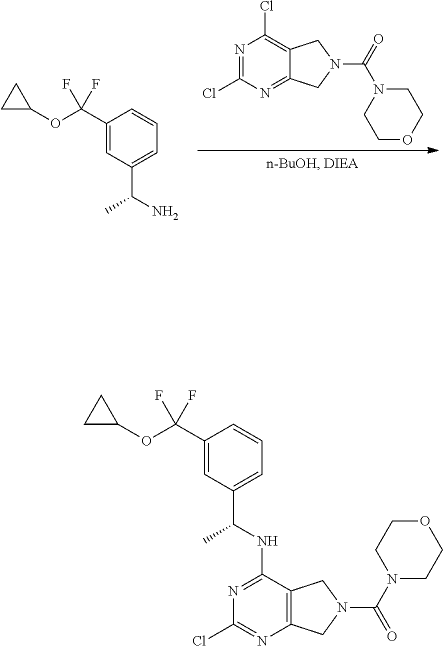 [SMILES: C[C@@H](N)c1cccc(C(F)(F)OC2CC2)c1 ; O=C(N1CCOCC1)N1Cc2nc(Cl)nc(Cl)c2C1 ; C[C@@H](Nc1nc(Cl)nc2c1CN(C(=O)N1CCOCC1)C2)c1cccc(C(F)(F)OC2CC2)c1]

### Step 1.

P₂S₅(1.01 g, 4.56 mmol, 485.26 μL) and trimethyl(trimethylsilyloxy)silane (2.96 g, 18.25 mmol, 3.88 mL) were added to the solution of cyclopropyl 3-bromobenzoate (4.4 g, 18.25 mmol) in XYLENE (44 mL) and the solution was stirred at 140° C. for 12 h. The reaction was cooled to 0° C., poured into aq.K₂CO₃ and extracted with MTBE. The combined organic layers were washed with brine, dried over Na₂SO₄ and the solvent was removed under reduced pressure. The crude residue was purified by column chromatography on silica gel to give O-cyclopropyl 3-bromobenzenecarbothioate (3.2 g, 68.18% yield). ¹H NMR (400 MHz, CDCl₃) (ppm 8.27 (t, J=2.0 Hz, 1H), 8.10 (t, J=8.0 Hz, 1H), 7.68 (t, J=7.2 Hz, 1H), 7.30 (t, J=4.0 Hz, 1H), 4.78-4.80 (m, 1H), 0.99-1.02 (m, 4H).

### Step 2.

SbCl₃ (47.90 mg, 210.00 μmol) and 2-methoxy-N-(2-methoxyethyl)-N-(trifluoro-sulfanyl)ethanamine (650.44 mg, 2.94 mmol, 644.00 μL) were added to the solution of O-cyclopropyl 3-bromobenzenecarbothioate (0.54 g, 2.10 mmol) in DCM (6 mL). The solution was stirred at 25° C. for 2 h. The reaction was poured into water and extracted with DCM. The combined organic layers were washed with brine, dried over Na₂SO₄ and the solvent was removed under reduced pressure. The crude residue was purified by column chromatography on silica gel (Petroleum ether: EtOAc=1:0 to 0:1) to give 1-bromo-3-[cyclopropoxy(difluoro)methyl]benzene (0.5 g, 90.50% yield). ¹H NMR (400 MHz, CDCl₃) (ppm 7.74 (s, 1H), 7.60 (d, J=8.0 Hz, 1H), 7.53 (d, J=8.0 Hz, 1H), 7.30 (t, J=8.0 Hz, 1H), 3.91-3.94 (m, 1H), 0.84-0.88 (m, 2H), 0.67-0.72 (m, 2H).

### Step 3.

To a mixture of 1-bromo-3-[cyclopropoxy(difluoro)methyl]benzene (0.4 g, 1.52 mmol) in DMF (4 mL) was added Pd(PPh₃)₂Cl₂ (53.36 mg, 76.02 μmol) and tributyl(1-ethoxyvinyl)stannane (658.94 mg, 1.82 mmol, 615.83 μL). The mixture was heated to 100° C. and stirred under N₂ for 2 h. Saturated aqueous CsF was added and the mixture was stirred for 10 min. The aqueous phase was extracted with MTBE, the combined organic phase was washed with brine, dried over Na₂SO₄ and the solvent was removed under reduced pressure to give 1-[cyclopropoxy(difluoro)-methyl]-3-(1-ethoxyvinyl)benzene (0.75 g, crude). LCMS (ESI): m/z: [M+H] calculated for C₁₄H₁₇F₂O₂: 255.12. found 255.2.

### Step 4.

A solution of 1-[cyclopropoxy(difluoro)methyl]-3-(1-ethoxyvinyl)benzene (0.75 g, 2.95 mmol) in aq. HCl (2 M, 7.50 mL) was stirred at 20° C. for 1 h. The mixture was poured into water and extracted with EtOAc. The combined organic layers were washed with brine, dried over Na₂SO₄ and the solvent was removed under reduced pressure. The crude residue was purified by column chromatography on silica gel to give 1-[3-[cyclopropoxy(difluoro)methyl]phenyl]ethanone (0.13 g, 19.48% yield). ¹H NMR (400 MHz, CDCl₃) δ ppm 8.17 (s, 1H), 8.06 (d, J=7.6 Hz, 1H), 7.80 (d, J=7.6 Hz, 1H), 7.54 (t, J=7.6 Hz, 1H), 3.95-3.97 (m, 1H), 2.64 (s, 3H), 0.86-0.95 (m, 2H), 0.70-0.73 (m, 2H).

### Step 5.

To a mixture of 1-[3-[cyclopropoxy(difluoro)methyl]phenyl]ethanone (100 mg, 442.05 μmol) and (R)-2-methylpropane-2-sulfinamide (107.15 mg, 884.10 μmol) in THF (2 mL) was added Ti(OEt)₄ (403.34 mg, 1.77 mmol, 366.67 μL). Then the mixture was stirred at 70° C. for 3 h. After cooling to 0° C., MeOH (14.16 mg, 442.05 μmol, 17.89 μL) and LiBH₄ (38.52 mg, 1.77 mmol, 1.50 mL) were added and the mixture was stirred at 0° C. for 1 h. Water was added and the mixture was extracted with EtOAc. The combined organic layers were washed with brine, dried over Na₂SO₄ and the solvent was removed under reduced pressure. The crude residue was purified by prep-TLC give N-[(1R)-1-[3-[cyclopropoxy(difluoro)methyl]phenyl]ethyl]-2-methyl-propane-2-sulfinamide (0.12 g, 81.91% yield). ¹H NMR (400 MHz, CDCl₃) (ppm 7.56 (s, 1H), 7.52 (d, J=7.6 Hz, 1H), 7.47 (d, J=8.0 Hz, 1H), 7.41 (t, J=7.2 Hz, 1H), 4.58-4.61 (m, 1H), 3.92-3.94 (m, 1H), 3.41 (s, 1H), 1.53 (d, J=6.4 Hz, 3H), 1.25 (s, 9H), 0.85-0.88 (m, 2H), 0.68-0.70 (m, 2H).

### Step 6.

HCl/MeOH (4 M, 181.04 μL) was added to a solution of N-[(1R)-1-[3-[cyclopropoxy(difluoro)methyl]phenyl]ethyl]-2-methyl-propane-2-sulfinamide (0.12 g, 362.08 μmol) in MeOH (2.4 mL). The solution was stirred at 20° C. for 2 h. The pH of the mixture was adjusted to 8 with. NaOH in MeOH. The solvent was removed under reduced pressure and the residue was added to a solution of 6 mL (MeOH: DCM=1:5). The suspension was stirred at 20° C. for 10 min and the solvent was removed under reduced pressure to give (1R)-1-[3-[cyclopropoxy(difluoro) methyl]phenyl]ethanamine (0.1 g, crude. ¹H NMR (400 MHz, CDCl₃) δ ppm 8.61 (br s, 2H), 7.68 (s, 1H), 7.64 (d, J=8.0 Hz, 1H), 7.57 (d, J=8.0 Hz, 1H), 7.42 (t, J=7.6 Hz, 1H), 4.39-4.46 (m, 1H), 3.91-3.93 (m, 1H), 1.66 (d, J=6.8 Hz, 3H), 0.83-0.87 (m, 2H), 0.63-0.68 (m, 2H).

### Step 7.

To a solution of (1R)-1-[3-[cyclopropoxy(difluoro)methyl]phenyl]ethanamine (0.1 g, 440.04 μmol) and (2,4-dichloro-5,7-dihydropyrrolo[3,4-d]pyrimidin-6-yl)-morpholino-methanone (160.08 mg, 528.05 μmol) in n-BuOH (2 mL) was added DIEA (284.36 mg, 2.20 mmol, 383.24 μL). The mixture was stirred at 100° C. for 3 h. After cooling to rt the mixture was poured into water and extracted with EtOAc. The combined organic layers were washed with brine, dried over Na₂SO₄ and the solvent was removed under reduced pressure. The crude residue was purified by prep-HPLC to give [2-chloro-4-[[(1R)-1-[3-[cyclopropoxy(difluoro)methyl]phenyl]ethyl] amino]-5,7-dihydropyrrolo[3,4-d]pyrimidin-6-yl]-morpholino-methanone (63 mg, 25.56% yield). LCMS (ESI): m/z: [M+H] calculated for C₂₃H₂₇ClF₂N5O₃: 494.17. found 494.3; ¹H NMR (400 MHz, CDCl₃) δ ppm 7.60 (s, 1H), 7.53 (d, J=8.0 Hz, 1H), 7.48 (d, J=7.2 Hz, 1H), 7.42 (t, J=8.0 Hz, 1H), 5.45-5.46 (m, 1H), 4.87-4.88 (m, 1H), 4.57-4.59 (m, 4H), 3.92-3.95 (m, 1H), 3.73 (t, J=4.8 Hz, 4H), 3.34 (t, J=4.8 Hz, 4H), 1.63 (d, J=7.2 Hz, 3H), 0.84-0.88 (m, 2H), 0.66-0.71 (m, 2H).

### Example 525. Synthesis of N-[(1R)-1-[3-(2-amino-1,1-difluoroethyl)phenyl]ethyl]-2-chloro-6-(4-methoxyoxane-4-carbonyl)-5H,6H,7H-pyrrolo[3,4-d]pyrimidin-4-amine

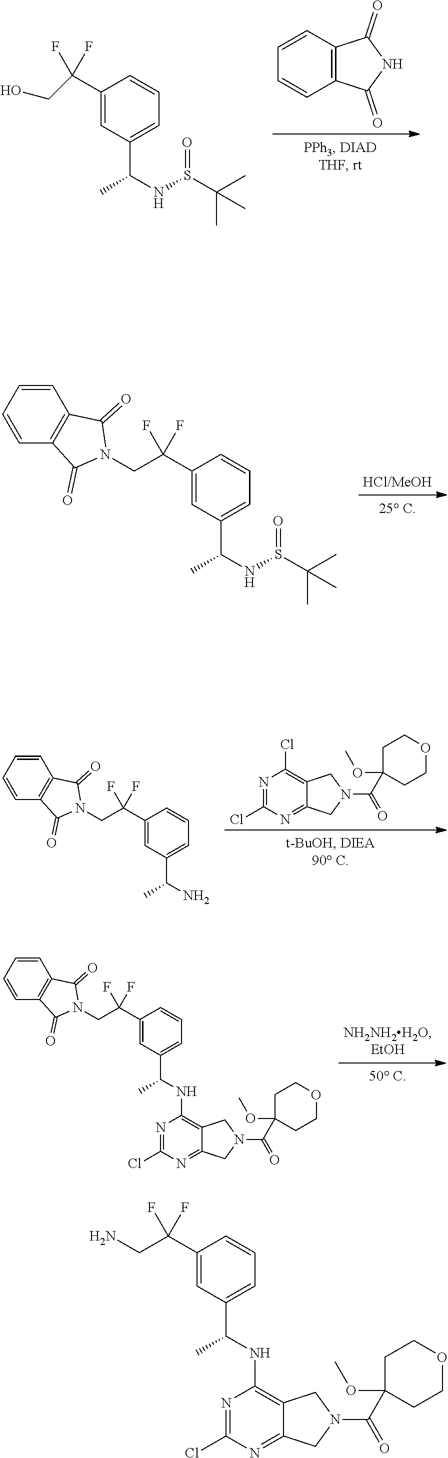

### Step 1.

To a solution of N-[(1R)-1-[3-(1,1-difluoro-2-hydroxy-ethyl)phenyl]ethyl]-2-methyl-propane-2-sulfinamide (2 g, 6.55 mmol) and isoindoline-1,3-dione (1.93 g, 13.10 mmol) in THF (20 mL) was added PPh₃ (3.44 g, 13.10 mmol) and DIAD (2.65 g, 13.10 mmol, 2.55 mL) dropwise at −78° C. The reaction was stirred at 25° C. for 12 h, the solvent was removed under reduced pressure and the residue was taken up in H₂O and extracted with EtOAc. The combined organic layers were washed with brine and dried over Na₂SO₄. The solvent was removed under reduced pressure and the crude residue was purified by prep-HPLC to give N-[(1R)-1-[3-[2-(1,3-dioxoisoindolin-2-yl)-1,1-difluoro-ethyl]phenyl]ethyl]-2-methyl-propane-2-sulfinamide (380 mg, 12% yield). LCMS (ESI): m/z: [M+H] calculated for C₂₂H₂₅F₂N₂O₃S: 435.1. found 435.2.

To a solution of N-[(1R)-1-[3-[2-(1,3-dioxoisoindolin-2-yl)-1,1-difluoro-ethyl]phenyl]ethyl]-2-methyl-propane-2-sulfinamide (350 mg, 805.53 μmol) in MeOH (3.5 mL) was added HCl/MeOH (4 M, 402.76 μL). The mixture was stirred at 25° C. for 2 h and then adjusted to pH=7 ˜ 8 using sat. NaOH/MeOH. The solvent was removed under reduced pressure to give 2-[2-[3-[(1R)-1-aminoethyl]phenyl]-2,2-difluoro-ethyl]isoindoline-1,3-dione (266 mg, crude), which was used without further purification. LCMS (ESI): m/z: [M+H] calculated for C₁₈H₁₇F₂N₂O₂: 331.1. found 331.2.

### Step 3.

To a solution of 2-[2-[3-[(1R)-1-aminoethyl]phenyl]-2,2-difluoro-ethyl]isoindoline-1,3-dione (266 mg, 805.26 μmol) and (2,4-dichloro-5,7-dihydropyrrolo[3,4-d]pyrimidin-6-yl)-(4-methoxytetrahydropyran-4-yl)methanone (267.49 mg, 805.26 μmol) in t-BuOH (1 mL) was added DIEA (1.04 g, 8.05 mmol, 1.40 mL). The mixture was stirred at 90° C. for 8 h, cooled to rt and the solvent was removed under reduced pressure. The residue was diluted with H₂O and extracted with EtOAc. The combined organic layers were washed with brine, dried over Na₂SO₄ and the solvent was removed under reduced pressure. The crude residue was purified by column chromatography to give 2-[2-[3-[(1R)-1-[[2-chloro-6-(4-methoxytetrahydropyran-4-carbonyl)-5,7-dihydropyrrolo[3,4-d]pyrimidin-4-yl]amino]ethyl]phenyl]-2,2-difluoro-ethyl]isoindoline-1,3-dione (260 mg, 51.57% yield). LCMS (ESI): m/z: [M+H] calculated for C₃₁H₃₁ClF₂N₅O₅: 626.2. found 626.4.

### Step 4.

To a solution of 2-[2-[3-[(1R)-1-[[2-chloro-6-(4-methoxytetrahydropyran-4-carbonyl)-5,7-dihydropyrrolo[3,4-d]pyrimidin-4-yl]amino]ethyl]phenyl]-2,2-difluoro-ethyl]isoindoline-1,3-dione (100 mg, 159.73 μmol) in EtOH (0.5 mL) was added NH₂NH₂.H₂O (8.00 mg, 159.73 μmol, 7.76 μL). The mixture was stirred at 50° C. for 2 h. Water was added and the mixture was directly purified by pre-HPLC to give [4-[[(1R)-1-[3-(2-amino-1,1-difluoro-ethyl)phenyl]ethyl]amino]-2-chloro-5,7-dihydropyrrolo[3,4-d]pyrimidin-6-yl]-(4-methoxytetrahydropyran-4-yl)methanone (10 mg, 12.62% yield). LCMS (ESI): m/z: [M+H] calculated for C₂₃H₂₉ClF₂N₅O₃: 496.2. found 496.2; ¹H NMR (400 MHz, METHANOL-d₄) δ ppm 7.58 (s, 1H) 7.52 (d, J=7.2 Hz, 1H) 7.39-7.46 (m, 2H) 5.40-5.43 (m, 1H) 4.96 (s, 1H) 4.87 (s, 1H) 4.61 (d, J=16.4 Hz, 2H) 3.76-3.77 (m, 4H) 3.16-3.27 (m, 5H) 1.92-2.10 (m, 4H) 1.59 (d, J=7.2 Hz, 3H).

### Example 526. Synthesis of N-[(1R)-1-[3-(2-amino-1,1-difluoroethyl)-2-fluorophenyl]ethyl]-2-chloro-6-(4-methoxyoxane-4-carbonyl)-5H,6H,7H-pyrrolo[3,4-d]pyrimidin-4-amine

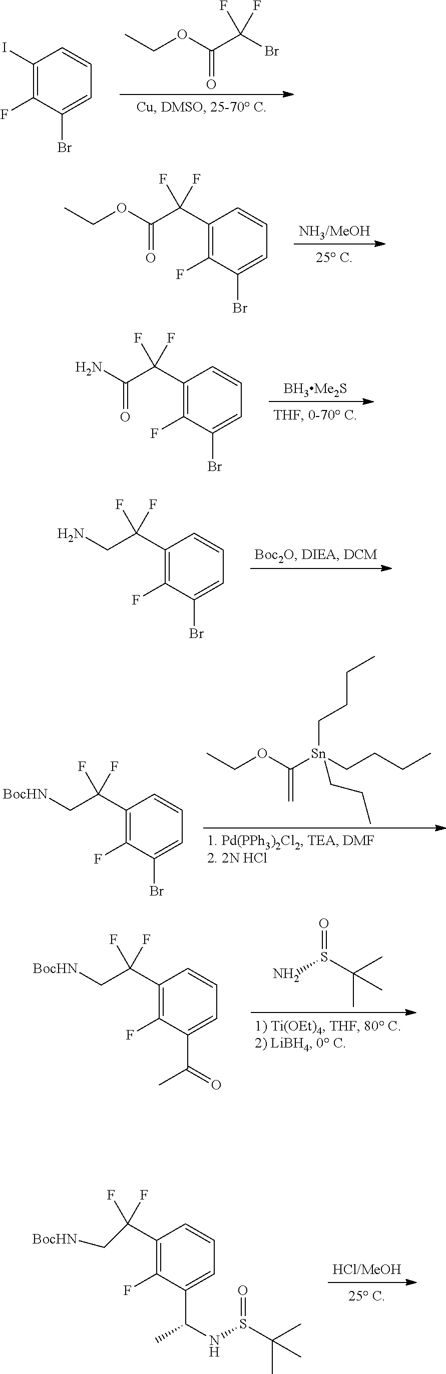

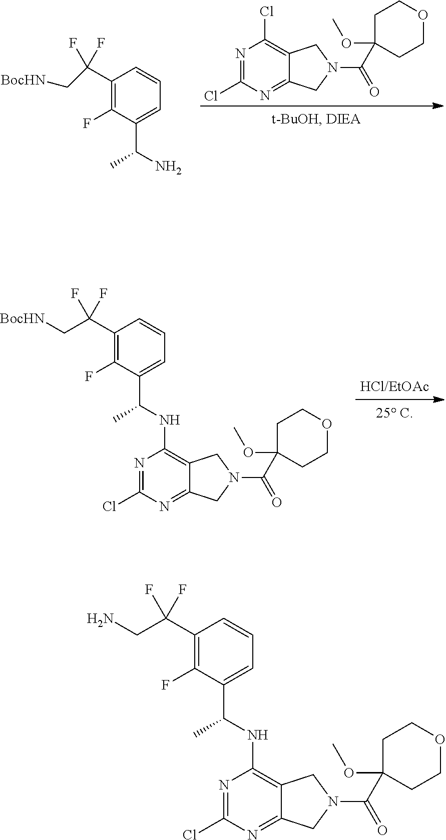 [SMILES: C[C@@H](N)c1cccc(C(F)(F)CNC(=O)OC(C)(C)C)c1F ; COC1(C(=O)N2Cc3nc(Cl)nc(Cl)c3C2)CCOCC1 ; COC1(C(=O)N2Cc3nc(Cl)nc(N[C@H](C)c4cccc(C(F)(F)CNC(=O)OC(C)(C)C)c4F)c3C2)CCOCC1 ; COC1(C(=O)N2Cc3nc(Cl)nc(N[C@H](C)c4cccc(C(F)(F)CN)c4F)c3C2)CCOCC1]

### Step 1.

To a solution of ethyl 2-bromo-2,2-difluoro-acetate (10.12 g, 49.85 mmol, 6.40 mL) in DMSO (70 mL) was added Cu (3.80 g, 59.82 mmol, 424.27 μL) at 25° C. The reaction mixture was stirred at 25° C. for 1h, after which 1-bromo-2-fluoro-3-iodo-benzene (6 g, 19.94 mmol) was added. The resulting reaction mixture was stirred at 70° C. for 3 h. The reaction was allowed to cool to room temperature and then diluted with H₂O (60 mL). The mixture was filtered and the solution was extracted with EtOAc. The combined organic phase was washed with brine, dried over Na₂SO₄, filtered and concentrated in vacuo. The resulting residue was purified by silica gel chromatography to afford ethyl 2-(3-bromo-2-fluoro-phenyl)-2,2-difluoro-acetate (4.8 g, 81.03% yield). ¹H NMR (400 MHz, CDCl₃) δ ppm 7.68-7.72 (m, 1H) 7.59 (t, J=6.4 Hz, 1H) 7.15 (t, J=8.0 Hz, 1H) 4.37 (q, J=7.2 Hz, 2H) 1.34 (t, J=7.2 Hz, 3H).

### Step 2.

A solution of ethyl 2-(3-bromo-2-fluoro-phenyl)-2,2-difluoro-acetate (4.8 g, 16.16 mmol) in 5 M NH₃/MeOH (50 mL) was stirred at 25° C. for 2 h. The reaction was concentrated under reduced pressure to afford 2-(3-bromo-2-fluoro-phenyl)-2,2-difluoro-acetamide (4.2 g, 96.98% yield). ¹H NMR (400 MHz, CDCl₃) δ ppm 7.70 (t, J=7.2 Hz, 1H) 7.62 (t, J=6.8 Hz, 1H) 7.15 (t, J=7.6 Hz, 1H) 6.25 (br d, J=205.6 Hz, 2H)

### Step 3.

Borane methylsulfide (10 M, 10.97 mL) was added dropwise to a solution of 2-(3-bromo-2-fluoro-phenyl)-2,2-difluoro-acetamide (4.2 g, 15.67 mmol) in THF (42 mL) at 0° C. The reaction mixture was stirred at 0° C. for 0.5 h. The mixture was then stirred at 70° C. for 10 h. The reaction mixture was cooled to 25° C. and quenched by dropwise addition of 2N HCl (50 mL) until the pH was adjusted to pH=9. The mixture was extracted with EtOAc and washed with brine. The organic layer was dried over anhydrous sodium sulfate and concentrated in vacuo. The resulting residue was purified by silica gel chromatography to afford 2-(3-bromo-2-fluoro-phenyl)-2,2-difluoro-ethanamine (2.5 g, 62.80% yield). LCMS (ESI): m/z: [M+H] calculated for C₈H₈BrF₃N: 253.97. found 254.0;

### Step 4.

To a solution of 2-(3-bromo-2-fluoro-phenyl)-2,2-difluoro-ethanamine (2.4 g, 9.45 mmol) in THF (25 mL) was added DIPEA (2.44 g, 18.89 mmol, 3.29 mL) and Boc₂O (2.06 g, 9.45 mmol, 2.17 mL). The mixture was stirred at 25° C. for 3 h. The reaction was diluted with water and then extracted with EtOAc. The combined organic layers were washed with saturated NaCl aqueous (30 mL), dried over Na₂SO₄, filtered and concentrated under reduced pressure to afford tert-butyl N-[2-(3-bromo-2-fluoro-phenyl)-2,2-difluoro-ethyl]carbamate (3.0 g, 84.28% yield, 94% purity). ¹H NMR (400 MHz, CDCl₃) δ ppm 7.65 (t, J=6.4 Hz, 1H) 7.47 (t, J=6.8 Hz, 1H) 7.09 (t, J=7.6 Hz, 1H) 4.83-4.85 (m, 1H) 3.82-3.90 (m, 2H) 1.36 (s, 9H)

### Step 5.

To a solution of tert-butyl N-[2-(3-bromo-2-fluoro-phenyl)-2,2-difluoro-ethyl]carbamate (3.0 g, 8.47 mmol) in DMF (40 mL) was added Pd(PPh₃)₂Cl₂ (297.28 mg, 423.53 μmol) and tributyl(1-ethoxyvinyl)stannane (4.28 g, 11.86 mmol, 4.00 mL) in one portion at 20° C. under N₂. The mixture was heated to 100° C. for 2 h. Saturated CsF (50 mL) was added and the resulting mixture was stirred for 10 min, and then filtered. The aqueous phase was extracted with EtOAc. The combined organic phase was washed with brine, dried over anhydrous Na₂SO₄, filtered and concentrated in vacuum. 1 N HCl (40 mL) aqueous was then added to the mixture. The reaction was stirred at 25° C. for 2 h. The mixture was extracted with EtOAc. The combined organic phase was washed with brine, dried over anhydrous Na₂SO₄, filtered and concentrated in vacuum. The remaining residue was purified by column chromatography to afford tert-butyl N-[2-(3-acetyl-2-fluoro-phenyl)-2,2-difluoro-ethyl]carbamate (1.65 g, 61.39% yield). ¹H NMR (400 MHz, CDCl₃) δ ppm 7.90-8.10 (m, 1H) 7.69-7.72 (m, 1H) 7.28-7.30 (m, 1H) 7.85-7.87 (m, 1H) 3.88-3.97 (m, 2H) 2.68 (d, J=5.6 Hz, 3H) 1.33 (s, 9H)

### Step 6.

To a solution of tert-butyl N-[2-(3-acetyl-2-fluoro-phenyl)-2,2-difluoro-ethyl]carbamate (300 mg, 945.47 μmol), and 2-methylpropane-2-sulfinamide (229.18 mg, 1.89 mmol) in THF (4 mL) was added Ti(OEt)₄ (862.68 mg, 3.78 mmol, 784.25 μL). The mixture was stirred at 80° C. for 4 h. Then the reaction was cooled to 0° C. and the LiBH₄ (20.60 mg, 945.47 μmol, 472.73 μL) was added to the mixture. The reaction was stirred at 0° C. for 1 h. The reaction was diluted with water (2 mL), and filtered. The resulting filtrate was extracted with EtOAc. The combined organic phase was washed with brine, dried over anhydrous Na₂SO₄, filtered and concentrated in vacuum. The residue was purified by silica gel chromatography to afford tert-butyl N-[2-[3-[(1R)-1-[[(R)-tert-butylsulfinyl]amino]ethyl]-2-fluoro-phenyl]-2,2-difluoro-ethyl]carbamate (120 mg, 30.04% yield). ¹H NMR (400 MHz, CDCl₃) δ ppm 7.45-7.50 (m, 2H) 7.10-7.22 (m, 1H) 4.81-4.85 (m, 1H) 3.84-3.95 (m, 2H) 1.56 (d, J=6.8 Hz, 3H) 1.35 (s, 9H) 1.24 (s, 9H)

### Step 7.

To a solution of tert-butyl N-[2-[3-[(1R)-1-[[(R)-tert-butylsulfinyl]amino]ethyl]-2-fluoro-phenyl]-2,2-difluoro-ethyl]carbamate (120 mg, 284.02 μmol) in MeOH (1 mL) was added HCl/MeOH (4 M, 142.01 μL). The mixture was stirred at 25° C. for 2 h. The pH was then adjusted to 7 by the addition of sat. NaOH/MeOH solution. The resulting mixture was concentrated in vacuum. The remaining residue was stirred with DCM/MeOH (5:1) (5 mL) for 10 min and then filtered and concentrated in vacuum to afford tert-butyl N-[2-[3-[(1R)-1-aminoethyl]-2-fluoro-phenyl]-2,2-difluoro-ethyl]carbamate (90 mg, 282.72 μmol, 99.54% yield). LCMS (ESI): m/z: [M+H] calculated for C₁₅H₂₂F₃N₂O₂: 319.16. found 319.2.

### Step 8.

To a solution of tert-butyl N-[2-[3-[(1R)-1-aminoethyl]-2-fluoro-phenyl]-2,2-difluoro-ethyl]carbamate (90 mg, 282.72 μmol) in n-BuOH (1 mL) was added (2,4-dichloro-5,7-dihydropyrrolo[3,4-d]pyrimidin-6-yl)-(4-methoxytetrahydropyran-4-yl)methanone (103.31 mg, 310.99 μmol) and DIEA (146.16 mg, 1.13 mmol, 196.98 μL). The mixture was stirred at 80° C. for 2 h. The reaction mixture was quenched by water (1 mL), and then extracted with EtOAc. The combined organic layers were washed with brine, dried over Na₂SO₄, filtered and concentrated under reduced pressure. The resulting residue was purified by column chromatography to afford tert-butyl N-[2-[3-[(1R)-1-[[2-chloro-6-(4-methoxytetrahydropyran-4-carbonyl)-5,7-dihydropyrrolo[3,4-d]pyrimidin-4-yl]amino]ethyl]-2-fluoro-phenyl]-2,2-difluoro-ethyl]carbamate (120 mg, 69.12% yield). LCMS (ESI): m/z: [M+H] calculated for C₂₈H₃₆ClF₃N₅O₅: 614.24. found 614.3; RT=0.859 min. ¹H NMR (400 MHz, MeOH) δ ppm 7.52 (m, 1H) 7.41 (m, 1H) 7.18 (m, 1H) 5.57-5.60 (m, 1H) 4.90-5.00 (m, 2H) 4.56-4.67 (m, 2H) 3.65-3.77 (m, 6H) 3.24 (d, J=14.8 Hz, 3H) 2.04-2.10 (m, 2H) 1.86-1.96 (m, 2H) 1.60-1.63 (m, 3H) 1.22 (s, 9H)

### Step 9.

To a solution of tert-butyl N-[2-[3-[(1R)-1-[[2-chloro-6-(4-methoxytetrahydropyran-4-carbonyl)-5,7-dihydropyrrolo[3,4-d]pyrimidin-4-yl]amino]ethyl]-2-fluoro-phenyl]-2,2-difluoro-ethyl]carbamate (120 mg, 195.42 μmol) in EtOAc (1 mL) was added HCl/EtOAc (4 M, 97.71 μL). The reaction was stirred at 25° C. for 2 h. The reaction was concentrated under reduced pressure and the resulting residue was purified by prep-HPLC to afford [4-[[(1R)-1-[3-(2-amino-1,1-difluoro-ethyl)-2-fluoro-phenyl]ethyl]amino]-2-chloro-5,7-dihydropyrrolo[3,4-d]pyrimidin-6-yl]-(4-methoxytetrahydropyran-4-yl)methanone (38 mg, 37.59% yield). LCMS (ESI): m/z: [M+H] calculated for C₂₃H₂₈ClF₃N₅O₃: 514.18. found 514.3; ¹H NMR (400 MHz, CDCl₃) δ ppm 7.45-7.51 (m, 2H) 7.19-7.24 (m, 1H) 5.58-5.60 (m, 1H) 4.87-5.12 (m, 1H) 4.84 (d, J=15.6 Hz, 2H) 4.67 (d, J=23.6 Hz, 2H) 3.72-3.82 (m, 4H) 3.33 (t, J=16.0 Hz, 2H) 3.23 (d, J=24.0 Hz, 3H) 2.09-2.18 (m, 2H) 1.85-1.93 (m, 2H) 1.65 (d, J=7.2 Hz, 3H)

### Example 527. Synthesis of 2-chloro-N-[(1R)-1-[2-fluoro-3-(fluoromethyl)phenyl]ethyl]-6-(morpholine-4-carbonyl)-5H,6H,7H-pyrrolo[3,4-d]pyrimidin-4-amine

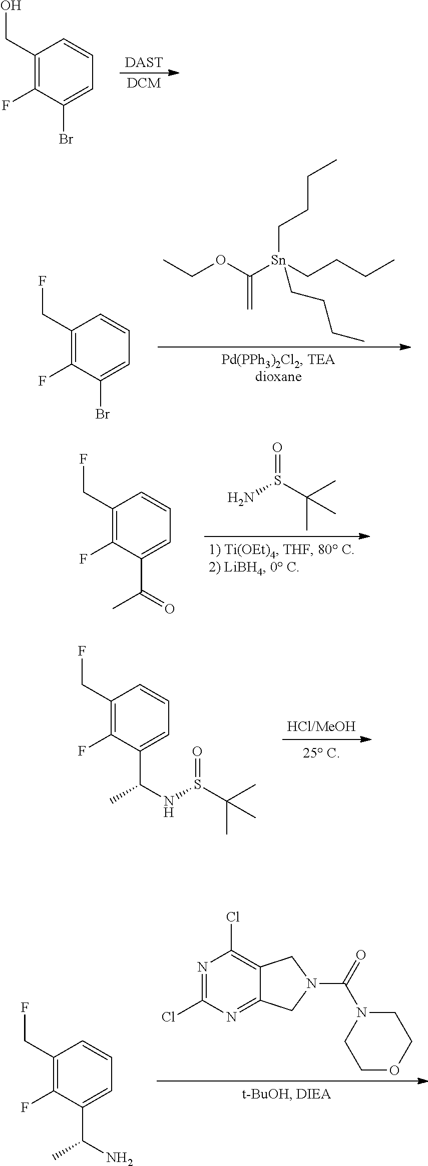

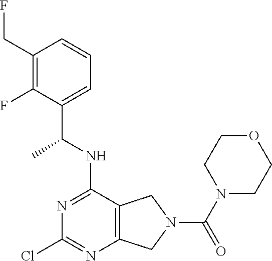 [SMILES: C[C@@H](Nc1nc(Cl)nc2c1CN(C(=O)N1CCOCC1)C2)c1cccc(CF)c1F]

### Step 1.

To a solution of (3-bromo-2-fluoro-phenyl)methanol (1 g, 4.88 mmol) in DCM (10 mL) was added DAST (1.57 g, 9.75 mmol, 1.29 mL) in DCM (10 mL) at 0° C. under N₂. The reaction mixture was then warmed to 25° C. and stirred for 2 h. The reaction was diluted with water and the aqueous phase was extracted with EtOAc. The combined organic phase was washed with brine, dried over anhydrous Na₂SO₄, filtered and concentrated in vacuum. The resulting residue was purified by column chromatography to afford 1-bromo-2-fluoro-3-(fluoromethyl)benzene (0.8 g, crude). ¹H NMR (400 MHz, METHANOL-d₄) δ ppm 7.62-7.66 (m, 1H), 7.43-7.47 (m, 1H), 7.13-7.17 (m, 1H), 5.48 (d, J=48.0 Hz, 2H).

To a solution of 1-bromo-2-fluoro-3-(fluoromethyl)benzene (0.5 g, 2.42 mmol) in DMF (3 mL) was added Pd(PPh₃)₂Cl₂ (84.76 mg, 120.76 μmol) and tributyl(1-ethoxyvinyl) stannane (1.05 g, 2.90 mmol, 978.25 μL) in one portion at 25° C. under N₂. The mixture was heated to 100° C. and stirred for 2 h. CsF aqueous (3 mL) was then added and the reaction was stirred for 10 min, and then filtered. The aqueous phase was extracted with EtOAc. The combined organic phase was washed with brine, dried over anhydrous Na₂SO₄, filtered and concentrated in vacuum. The resulting residue was dissolved in 2N HCl (10 mL), and stirred at 25° C. for 2 h. The mixture was purified by column chromatography to afford 1-[2-fluoro-3-(fluoromethyl)phenyl]ethanone (150 mg, 36.50% yield). ¹H NMR (400 MHz, METHANOL-d₄) δ ppm 7.87-7.95 (m, 1H), 7.70-7.73 (m, 1H), 7.31-7.36 (m, 1H), 5.53 (d, J=48.4 Hz 2H), 2.64 (d, J=4.8 Hz, 3H). LCMS (ESI): m/z: [M+H] calculated for C₉H₉F₂O: 171.05. found 171.2.

### Step 3.

To a solution of 1-[2-fluoro-3-(fluoromethyl)phenyl]ethanone (0.150 g, 881.55 μmol) in THF (5 mL) was added Ti(OEt)₄ (804.36 mg, 3.53 mmol, 731.23 μL) and 2-methylpropane-2-sulfinamide (213.69 mg, 1.76 mmol) at 25° C. under N₂. The mixture was stirred at 80° C. for 2 h. After cooling to 0° C., LiBH₄ (76.80 mg, 3.53 mmol) and MeOH (28.24 mg, 881.55 μmol, 35.67 μL) were added to the mixture and the mixture was stirred 1 h at 0° C. The residue was diluted with water and extracted with EtOAc. The combined organic phase was washed with brine, dried over anhydrous Na₂SO₄, filtered and concentrated in vacuum. The remaining residue was purified to afford N-[(1R)-1-[2-fluoro-3-(fluoromethyl)phenyl]ethyl]-2-methyl-propane-2-sulfinamide (100 mg, 41.20% yield). ¹H NMR (400 MHz, METHANOL-d₄) δ ppm 7.53-7.57 (m, 1H), 7.36-7.39 (m, 1H), 7.19-7.23 (m, 1H), 5.47 (d, J=49.2 Hz 2H), 4.79-4.82 (m, 1H), 1.52 (d, J=6.8 Hz, 3H), 1.21 (s, 9H). LCMS (ESI): m/z: [M+H] calculated for C₁₃H₂₀NF₂OS:276.12. found 276.2.

### Step 4.

To a solution of N-[(1R)-1-[2-fluoro-3-(fluoromethyl)phenyl]ethyl]-2-methyl-propane-2-sulfinamide (100 mg, 363.16 μmol) in MeOH (3 mL) was added HCl/MeOH (4 M, 181.58 μL) at 25° C. The mixture was stirred for 2 h after which the reaction mixture was concentrated. The residue was dissolved into MeOH (4 mL), adjusted to pH=7 with sat. solution of NaOH in MeOH and then concentrated. The remaining residue was dissolved in DCM and MeOH (5:1, 5 mL), filtered and concentrated to afford (1R)-1-[2-fluoro-3-(fluoromethyl)phenyl]ethanamine (50 mg, 80.43% yield). LCMS (ESI): m/z: [M+H] calculated for C₉H₁₂F₂N: 172.19. found 172.2. ¹H NMR (400 MHz, METHANOL-d₄) δ ppm 7.49-7.53 (m, 1H), 7.33-7.37 (m, 1H), 7.19-7.22 (m, 1H), 5.45 (d, J=48.0 Hz 2H), 4.27-4.39 (m, 1H), 1.41 (d, J=6.8 Hz, 3H).

### Step 5.

To a solution of (1R)-1-[2-fluoro-3-(fluoromethyl)phenyl]ethanamine (0.05 g, 292.08 μmol) and (2,4-dichloro-5,7-dihydropyrrolo[3,4-d]pyrimidin-6-yl)-morpholino-methanone (97.40 mg, 321.29 μmol) in n-BuOH (1 mL) was added DIEA (188.75 mg, 1.46 mmol, 254.37 μL) at 25° C. The reaction mixture was then stirred at 80° C. for 2 h. The mixture was filtered and purified by prep-HPLC to afford [2-chloro-4-[[(1R)-1-[2-fluoro-3-(fluoromethyl)phenyl]ethyl]amino]-5,7-dihydropyrrolo[3,4-d]pyrimidin-6-yl]-morpholino-methanone (59.3 mg, 135.43 μmol, 46.37% yield). LCMS (ESI): m/z: [M+H] calculated for C₂₀H₂₃ClF₂N₅O₂: 438.14. found 438.3. ¹H NMR (400 MHz, METHANOL-d₄) δ ppm 7.34-7.45 (m, 2H), 7.15-7.19 (m, 1H), 5.57-5.62 (m, 1H), 5.45 (d, J=48.0 Hz 2H), 4.61-4.64 (m, 2H), 4.56-4.57 (m, 1H), 4.55-4.59 (m, 1H), 3.66-3.73 (m, 4H), 3.34-3.37 (m, 4H), 1.55 (d, J=7.2 Hz, 3H)

### Example 528. Synthesis of N-[(1R)-1-[2-bromo-3-(difluoromethyl)phenyl]ethyl]-2-chloro-6-(morpholine-4-carbonyl)-5H,6H,7H-pyrrolo[3,4-d]pyrimidin-4-amine

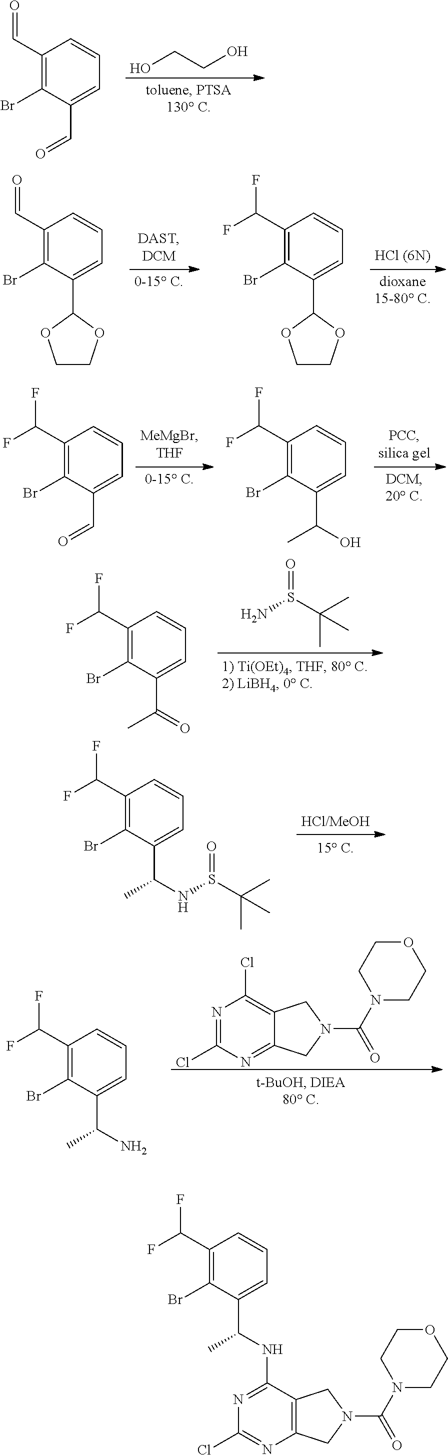

### Step 1.

To a solution of 2-bromobenzene-1,3-dicarbaldehyde (1.5 g, 7.04 mmol) in toluene (27 mL) was added 4-methylbenzenesulfonic acid hydrate (26.79 mg, 140.83 μmol) and the resulting mixture was heated to 130° C. Ethylene glycol (437.04 mg, 7.04 mmol, 393.73 μL) was added over 3 h and water generated from the reaction mixture was removed with a Dean-Stark trap. The mixture was then stirred at 130° C. for an additional 1 h. The reaction mixture was cooled to room temperature, washed with saturated NaHCO₃ solution and extracted with DCM. The organic phase was washed with brine, dried over anhydrous MgSO₄ and concentrated under vacuum. The resulting residue was purified by column chromatography to afford 2-bromo-3-(1,3-dioxolan-2-yl)benzaldehyde (1.1 g, 60.77% yield). LCMS (ESI): m/z: [M+H] calculated for C₁₀H₁₀BrO₃: 256.97. found 257.0. H NMR (400 MHz, DMSO-d₆) (ppm 10.33 (s, 1H), 7.86 (d, J=7.6 Hz, 2H), 7.58-7.62 (m, 1H), 6.11 (s, 1H), 4.02-4.11 (m, 4H).

### Step 2.

To a solution of 2-bromo-3-(1,3-dioxolan-2-yl)benzaldehyde (2 g, 7.78 mmol) in DCM (20 mL) was added N-ethyl-N-(trifluoro-sulfanyl)ethanamine (3.76 g, 23.34 mmol, 3.08 mL) at 0° C. under N₂. The resulting mixture was then stirred at 15° C. for 2 h. The solution was then poured into ice water and partitioned between saturated sodium bicarbonate and EtOAc. The mixture was extracted with EtOAc. The organic layers were combined and concentrated in vacuum. The resulting residue was purified by column chromatography to afford 2-[2-bromo-3-(difluoromethyl)phenyl]-1,3-dioxolane (1.67 g, 76.92% yield). ¹H NMR (400 MHz, CDCl₃) (ppm 7.74 (d, J=7.6 Hz, 1H), 7.69 (d, J=7.6 Hz, 1H), 7.47 (t, J=7.6 Hz, 1H), 7.00 (t, J=54.4 Hz, 1H), 6.17 (s, 1H), 4.10-4.18 (m, 4H).

### Step 3.

To a solution of 2-[2-bromo-3-(difluoromethyl)phenyl]-1,3-dioxolane (1.6 g, 5.73 mmol) in dioxane (16 mL) was added aq. HCl (6 M, 16.00 mL) at 15° C. and the resulting mixture was stirred at 80° C. for 10 h. The solution was poured into water and EtOAc and the aqueous layer was extracted with EtOAc. The combined organic phase was washed with brine, dried over anhydrous MgSO₄ and concentrated in vacuum. The residue was purified by column chromatography to afford 2-bromo-3-(difluoromethyl)benzaldehyde (1.15 g, 85.35% yield). ¹H NMR (400 MHz, CDCl₃) ppm 10.47 (s, 1H), 8.04 (d, J=7.6 Hz, 1H), 7.91 (d, J=7.6 Hz, 1H), 7.58 (t, J=7.6 Hz, 1H), 7.05 (t, J=54.4 Hz, 1H).

### Step 4.

To a solution of 2-bromo-3-(difluoromethyl)benzaldehyde (1.05 g, 4.47 mmol) in THF (10 mL) was added MeMgBr (3 M, 1.94 mL) at 0° C. under N₂ and the resulting mixture was stirred at 15° C. for 3 h. The reaction was poured onto ice water and acidified with 4N HCl aqueous to pH 2 ˜ 3. The resulting mixture was extracted with EtOAc and the combined organic layers were washed with brine, dried over Na₂SO₄, filtered and concentrated under reduced pressure to afford 1-[2-bromo-3-(difluoromethyl)phenyl]ethanol (1.05 g, 93.61% yield). ¹H NMR (400 MHz, CDCl₃) δ ppm 7.77 (d, J=7.6 Hz, 1H), 7.59 (d, J=7.6 Hz, 1H), 7.48 (t, J=7.6 Hz, 1H), 6.98 (t, J=54.8 Hz, 1H), 5.32-5.37 (m, 1H), 1.96 (br s, 1H), 1.51 (d, J=6.4 Hz, 3H)

### Step 5.

To a solution of 1-[2-bromo-3-(difluoromethyl)phenyl]ethanol (1 g, 3.98 mmol) in DCM (10 mL) was added PCC (2.58 g, 11.95 mmol) and Silica gel (2.58 g, 42.94 mmol) at 20° C. under N₂. The reaction mixture was stirred at 20° C. for 1 h. The reaction solution was filtered and concentrated under reduced pressure. The resulting residue was purified by column chromatography to afford 1-[2-bromo-3-(difluoromethyl)phenyl]ethanone (683 mg, 68.85% yield). ¹H NMR (400 MHz, CDCl₃) δ ppm 7.75 (dd, J=2.4, 7.6 Hz, 1H), 7.47-7.53 (m, 2H), 6.99 (t, J=54.8 Hz, 1H), 2.64 (s, 3H)

### Step 6.

To a solution of 1-[2-bromo-3-(difluoromethyl)phenyl]ethanone (680 mg, 2.73 mmol) in THF (8 mL) was added 2-methylpropane-2-sulfinamide (661.84 mg, 5.46 mmol) and Ti(OEt)₄ (2.49 g, 10.92 mmol, 2.26 mL) under N₂. The reaction mixture was stirred at 80° C. for 3 h after which LiBH₄ (59.48 mg, 2.73 mmol) was added to the reaction solution at 0° C. The resulting mixture was stirred at 0° C. for 2 h. The reaction was then poured into ice water. The aqueous layer was extracted with EtOAc and the combined organic layers were washed with brine, dried over Na₂SO₄, filtered and concentrated under reduced pressure. The remaining residue was purified by column chromatography to afford N-[(1R)-1-[2-bromo-3-(difluoromethyl)phenyl]ethyl]-2-methyl-propane-2-sulfinamide (350 mg, 35.46% yield). LCMS (ESI): m/z: [M+H] calculated for C13H₁₉BrF₂NOS: 354.03. found 354.1. ¹H NMR (400 MHz, METHANOL-d₄) (ppm 7.75 (d, J=7.6 Hz, 1H), 7.58 (d, J=6.4 Hz, 1H), 7.52 (t, J=7.6 Hz, 1H), 7.06 (t, J=54.8 Hz, 1H), 5.01-5.06 (m, 1H), 1.49 (d, J=6.8 Hz, 3H), 1.22 (s, 9H).

### Step 7.

A solution of N-[(1R)-1-[2-bromo-3-(difluoromethyl)phenyl]ethyl]-2-methyl-propane-2-sulfinamide (100 mg, 282.28 μmol) in HCl/MeOH (4 M, 1 mL) was stirred at 15° C. for 3 h. The reaction mixture was then adjusted to pH 7 with sat. solution of NaOH/MeOH after which the mixture was concentrated under reduced pressure. The resulting residue was dissolved in DCM (3 mL) and MeOH(0.6 mL) and the solution was filtered. The resulting organic layer was concentrated under reduced pressure to afford (1R)-1-[2-bromo-3-(difluoromethyl)phenyl]ethanamine (60 mg, 84.99% yield). LCMS (ESI): m/z: [M+H] calculated for C₉H₁₁BrF₂N: 250.00. found 250.0. ¹H NMR (400 MHz, METHANOL-d₄) (ppm 7.65-7.76 (m, 3H), 7.10 (t, J=54.4 Hz, 1H), 5.02-5.07 (m, 1H), 1.64 (d, J=6.8 Hz, 3H)

### Step 8.

To a solution of (1R)-1-[2-bromo-3-(difluoromethyl)phenyl]ethanamine (60 mg, 239.92 μmol) in t-BuOH (1 mL) was added (2,4-dichloro-5,7-dihydropyrrolo[3,4-d]pyrimidin-6-yl)-morpholino-methanone (72.73 mg, 239.92 μmol) and DIEA (93.02 mg, 719.76 μmol, 125.37 μL) under N₂. The reaction mixture was stirred at 80° C. for 2 h. The mixture was poured into water and the aqueous phase was extracted with EtOAc. The combined organic phase was washed with brine, dried over anhydrous Na₂SO₄, filtered and concentrated in vacuum. The remaining residue was purified by prep-HPLC to afford [4-[[(1R)-1-[2-bromo-3-(difluoromethyl)phenyl]ethyl]amino]-2-chloro-5,7-dihydropyrrolo[3,4-d]pyrimidin-6-yl]-morpholino-methanone (51 mg, 40.96% yield). LCMS (ESI): m/z: [M+H] calculated for C₂₀H₂₂BrClF₂N₅O₂: 516.05. found 516.1. H NMR (400 MHz, METHANOL-d₄) (ppm 7.55-7.58 (m, 2H), 7.45 (t, J=7.2 Hz, 1H), 7.10 (t, J=54.8 Hz, 1H), 5.68-5.72 (m, 1H), 4.57-4.66 (m, 4H), 3.69-3.77 (m, 4H), 3.35-3.39 (m, 4H), 1.55 (d, J=7.2 Hz, 3H)

### Example 529. Synthesis of 1-[2-chloro-4-[[(1R)-1-[3-(difluoromethyl)-2-fluoro-phenyl] ethyl]amino]-5,7-dihydropyrrolo[3,4-d]pyrimidin-6-ium-6-ylidene]-N,N,N′,N′-tetramethyl-methanediamine formate

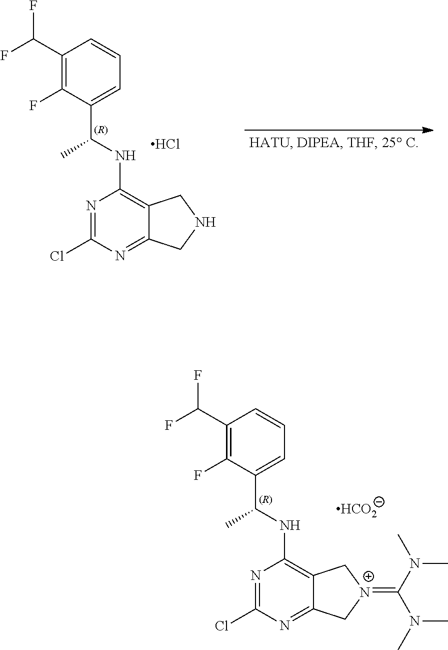

### Step 1.

To a mixture of 2-chloro-N-[(1R)-1-[3-(difluoromethyl)-2-fluoro-phenyl]ethyl]-6,7-dihydro-5H-pyrrolo[3,4-d]pyrimidin-4-amine hydrochloride (50 mg, 132 μmol) and (3-hydroxyoxetane-3-carbonyl)oxylithium (16.4 mg, 132 μmol) in THF (1 mL) were added HATU (11.7 μL, 198 μmol) and DIEA (68.9 μL, 396 μmol). The mixture was stirred at 25° C. for 10 h under N₂. The reaction mixture was then filtered and the filtrate was purified by prep-HPLC to give 1-[2-chloro-4-[[(1R)-1-[3-(difluoromethyl)-2-fluoro-phenyl]ethyl]amino]-5,7-dihydropyrrolo[3,4-d]pyrimidin-6-ium-6-ylidene]-N,N,N′,N′-tetramethyl-methanediamine formate (20 mg, 34% yield). LCMS (ESI): m/z: [M+H] calculated for C₂₀H₂₅ClF₃N₆: 441.2. found: 441.2; ¹H NMR (400 MHz, METHANOL-d₄) δ ppm 7.58 (t, J=7.2 Hz, 1H) 7.50 (t, J=6.8 Hz, 1H) 7.26 (t, J=7.2 Hz, 1H) 6.99 (t, J=54.8 Hz, 1H) 5.66-5.56 (m, 1H) 4.67 (br s, 2H) 4.58 (br s, 2H) 3.04 (s, 12H) 1.61 (d, J=6.8 Hz, 3H).

### Example 530. Synthesis of [5-[[(1R)-1-[3-(difluoromethyl)-2-fluoro-phenyl]ethyl]amino]imidazo[1,2-c]pyrimidin-2-yl]-piperazin-1-yl-methanone hydrochloride

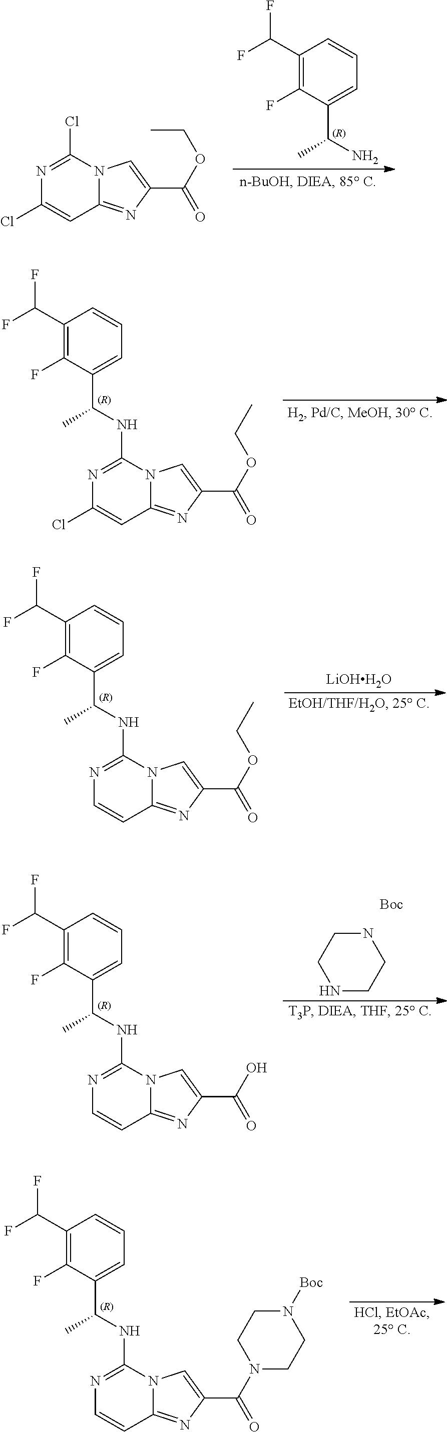

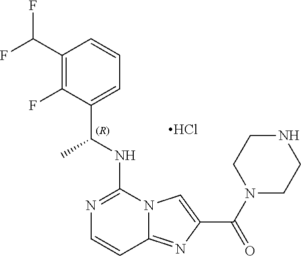

### Step 1.

To a mixture of ethyl 5,7-dichloroimidazo[1,2-c]pyrimidine-2-carboxylate (450 mg, 1.73 mmol) and (1R)-1-[3-(difluoromethyl)-2-fluoro-phenyl]ethanamine (327 mg, 1.73 mmol) in n-BuOH (1 mL) was added DIEA (1.12 g, 8.65 mmol). The reaction was stirred at 85° C. under N₂ for 3 h. The mixture was quenched by the addition of water (10 mL) and extracted with EtOAc. The combined organic extracts were dried over Na₂SO₄, filtered, and concentrated under reduced pressure. The crude residue was purified by column chromatography to give ethyl 7-chloro-5-[[(1R)-1-[3-(difluoromethyl)-2-fluoro-phenyl]ethyl]amino]imidazo[1,2-c]pyrimidine-2-carboxylate (640 mg, 90% yield). LCMS (ESI): m/z: [M+H] calculated for C₁₈H₁₇ClF₃N₄O₂: 413.1. found: 413.1.

To a solution of ethyl 7-chloro-5-[[(1R)-1-[3-(difluoromethyl)-2-fluoro-phenyl]ethyl]amino] imidazo[1,2-c]pyrimidine-2-carboxylate (640 mg, 1.55 mmol) in MeOH (1 mL) was added 10% Pd/C (43.6 mg, 31.01 μmol) under N₂. The suspension was degassed under vacuum and purged with H₂ gas three times. The mixture was stirred under H₂ (15 psi) at 30° C. for 3 h. The reaction mixture was then filtered and the filtrate was concentrated to give ethyl 5-[[(1R)-1-[3-(difluoromethyl)-2-fluoro-phenyl]ethyl]amino]imidazo[1,2-c]pyrimidine-2-carboxylate (580 mg, 99% yield). LCMS (ESI): m/z: [M+H] calculated for C₁₈H₁₈F₃N₄O₂: 379.1. found: 379.1; ¹H NMR (400 MHz, METHANOL-d₄) δ ppm 9.01 (s, 1H) 8.10 (d, J=7.2 Hz, 1H) 7.67 (t, J=7.2 Hz, 1H) 7.52 (t, J=7.2 Hz, 1H) 7.27 (t, J=7.6 Hz, 1H) 6.86-7.14 (m, 2H) 5.73 (q, J=7.2 Hz, 1H) 4.50 (q, J=7.2 Hz, 2H) 1.74 (d, J=7.2 Hz, 3H) 1.45 (t, J=7.2 Hz, 3H).

### Step 3.

To a mixture of ethyl 5-[[(1R)-1-[3-(difluoromethyl)-2-fluoro-phenyl]ethyl]amino]imidazo [1,2-c]pyrimidine-2-carboxylate (580 mg, 1.53 mmol) in EtOH (2 mL), THF (2 mL) and H₂O (2 mL) was added LiOH.H₂O (162 mg, 3.83 mmol). The mixture was stirred at 25° C. for 2 h under N₂. The reaction mixture was treated with a solution of HCl (2N in H₂O) until pH ˜ 4, then was extracted with CH₂Cl₂, dried over Na₂SO₄, filtered, and concentrated under reduced pressure to give 5-[[(1R)-1-[3-(difluoromethyl)-2-fluoro-phenyl]ethyl]amino]imidazo[1,2-c]pyrimidine-2-carboxylic acid (0.50 g, crude). LCMS (ESI): m/z: [M−H] calculated for C₁₋₆H₁₂F₃N₄O₂: 349.1. found 349.0; ¹H NMR (400 MHz, METHANOL-d₄) δ ppm 8.47 (s, 1H) 7.65-7.55 (m, 2H) 7.49 (t, J=6.8 Hz, 1H) 7.23 (t, J=7.6 Hz, 1H) 7.14-6.87 (m, 2H) 5.66 (q, J=6.4 Hz, 1H) 1.67 (d, J=7.2 Hz, 3H).

### Step 4.

To a mixture of 5-[[(1R)-1-[3-(difluoromethyl)-2-fluoro-phenyl]ethyl]amino]imidazo[1,2-c] pyrimidine-2-carboxylic acid (100 mg, 285 μmol) and tert-butyl piperazine-1-carboxylate (53.2 mg, 285 μmol) in THF (2 mL) were added T₃P (273 mg, 428 μmol) and DIEA (249 μL 1.43 mmol). The mixture was stirred at 25° C. for 10 h under N₂. The reaction mixture was quenched by water, extracted with EtOAc, dried over Na₂SO₄, filtered, and concentrated under reduced pressure. The crude residue was purified by column chromatography to give tert-butyl 4-[5-[[(1R)-1-[3-(difluoromethyl)-2-fluoro-phenyl]ethyl]amino]imidazo[1,2-c]pyrimidine-2-carbonyl]piperazine-1-carboxylate (120 mg, 81% yield). LCMS (ESI): m/z: [M+H] calculated for C₂₅H₃₀F₃N₆O₃: 519.2. found 519.2; ¹H NMR (400 MHz, METHANOL-d₄) δ ppm 8.44 (s, 1H) 7.63-7.59 (m, 2H) 7.50 (t, J=7.2 Hz, 1H) 7.24 (t, J=7.6 Hz, 1H) 7.00 (t, J=55.2 Hz, 1H) 6.81 (d, J=6.4 Hz, 1H) 5.66 (q, J=6.8 Hz, 1H) 3.98-3.77 (m, 4H) 3.53 (br s, 4H) 1.67 (d, J=6.8 Hz, 3H) 1.47 (s, 9H).

### Step 5.

tert-Butyl-4-[5-[[(1R)-1-[3-(difluoromethyl)-2-fluoro-phenyl]ethyl]amino]imidazo[1,2-c]pyrimidine-2-carbonyl]piperazine-1-carboxylate (120 mg, 231 μmol) was stirred in a 4M solution of HCl in EtOAc (578 μL, 2.31 mmol) at 25° C. for 1 h. The reaction mixture was then filtered to give [5-[[(1R)-1-[3-(difluoromethyl)-2-fluoro-phenyl]ethyl]amino]imidazo[1,2-c]pyrimidin-2-yl]-piperazin-1-yl-methanone hydrochloride (60 mg, 61% yield). LCMS (ESI): m/z: [M+H] calculated for C₂₀H₂₂F₃N₆O: 419.2. found: 419.2; ¹H NMR (400 MHz, METHANOL-d₄) δ ppm 9.12 (s, 1H) 8.12 (d, J=6.8 Hz, 1H) 7.77 (t, J=7.2 Hz, 1H) 7.52 (t, J=7.2 Hz, 1H) 7.27 (t, J=8.0 Hz, 1H) 7.14-7.00 (m, 2H) 5.76 (q, J=6.8 Hz, 1H) 4.14-4.08 (m, 4H) 3.42 (t, J=5.2 Hz, 4H) 1.76 (d, J=7.2 Hz, 3H).

### Example 531. Synthesis of [2-chloro-4-[[(1R)-1-[3-[2-(cyclopropoxy)-1,1-difluoro-ethyl]phenyl]ethyl]amino]-5,7-dihydropyrrolo[3,4-d]pyrimidin-6-yl]-morpholino-methanone

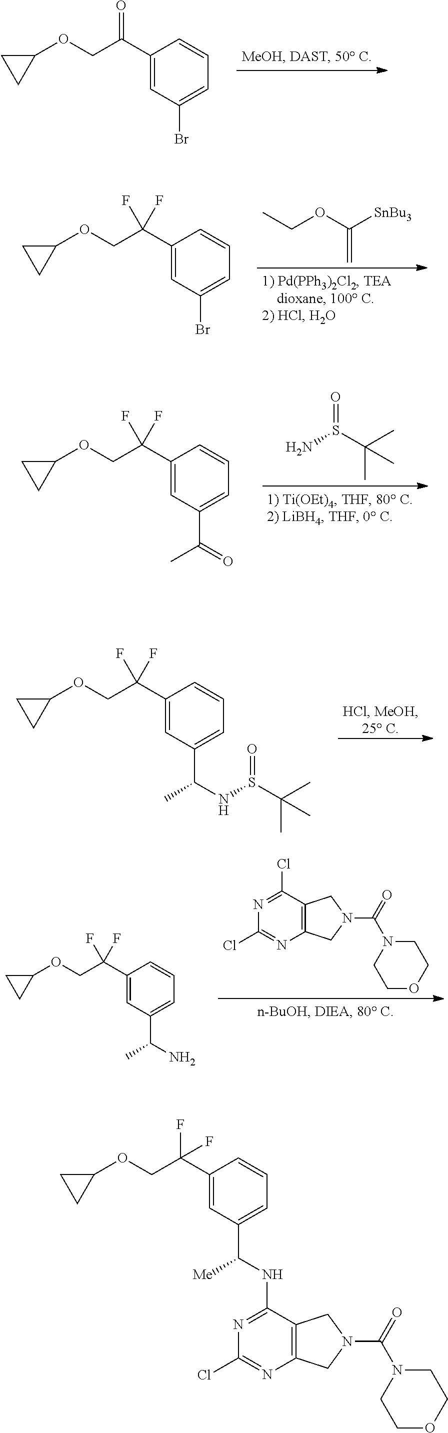

### Step 1.

To a solution of 1-(3-bromophenyl)-2-(cyclopropoxy)ethanone (430 mg, 1.69 mmol) in DAST (4.5 mL) was added MeOH (6.82 μL, 169 μmol) at 25° C. The mixture was heated to 50° C. and stirred for 12 h. The mixture was then diluted with ice water (10 mL) and extracted with EtOAc, treated with brine, dried over with Na₂SO₄, filtered, and concentrated under reduced pressure. The crude residue was purified by column chromatography to give 1-bromo-3-[2-(cyclopropoxy)-1,1-difluoro-ethyl]benzene (250 mg, 54% yield). ¹H NMR (400 MHz, CHLOROFORM-d) δ ppm 7.67 (s, 1H) 7.60-7.58 (m, 1H) 7.47-7.44 (m, 1H) 7.33-7.27 (m, 1H) 3.90 (t, J=12 Hz, 2H) 3.46-3.41 (m, 1H) 0.60-0.46 (m, 4H).

### Step 2.

To a solution of 1-bromo-3-[2-(cyclopropoxy)-1,1-difluoro-ethyl]benzene (100 mg, 361 μmol) and tributyl(1-ethoxyvinyl)stannane (183 μL, 541 μmol) in 1,4-dioxane (2 mL) were added TEA (126 μL, 902 μmol) and Pd(PPh₃)₂Cl₂ (25.3 mg, 36.1 μmol). The mixture was sparged with N₂ gas then was heated to 100° C. and stirred for 2 h. A 2M aqueous HCl solution (20 mL) was added and the mixture and stirred for 5 h. The mixture was filtered and the filtrate was extracted with EtOAc. The combined organic layers were poured into an aq. KF solution (10 mL) and stirred for 1 h. The mixture was filtered and the filtrate was treated with brine, dried over with Na₂SO₄, filtered, and concentrated under reduced pressure. The crude residue was purified by prep-TLC to give 1-[3-[2-(cyclopropoxy)-1,1-difluoro-ethyl]phenyl]ethanone (20 mg, 23% yield). ¹H NMR (400 MHz, CHLOROFORM-d) δ ppm 8.09 (s, 1H) 8.03 (d, J=8 Hz, 1H) 7.71 (d, J=8 Hz, 1H) 7.54 (t, J=8 Hz, 1H) 3.94 (t, J=14 Hz, 2H) 3.44-3.40 (m, 1H) 2.63 (s, 3H) 0.57-0.44 (m, 4H).

### Step 3.

To a solution of 1-[3-[2-(cyclopropoxy)-1,1-difluoro-ethyl]phenyl]ethanone (300 mg, 1.25 mmol) in THF (4 mL), (R)-2-Methyl-2-propanesulfinamide (303 mg, 2.50 mmol) and Ti(OEt)₄ (1.04 mL, 4.99 mmol) were added. The mixture was heated to 80° C. and stirred for 2 h. The mixture was cooled to 0° C., MeOH (50.5 μL, 1.25 mmol) and LiBH₄ (29.9 mg, 1.37 mmol) were added, and the resulting mixture was stirred for 1 h at 0° C. H₂O (10 mL), the mixture was filtered, and the filtrate was extracted with EtOAc. The combined organic extracts were treated with brine, dried with Na₂SO₄, filtered, and concentrated under reduced pressure. The residue was purified by column chromatography to give N-[(1R)-1-[3-[2-(cyclopropoxy)-1,1-difluoro-ethyl]phenyl]ethyl]-2-methyl-propane-2-sulfinamide (215 mg, 49% yield). ¹H NMR (400 MHz, CHLOROFORM-d) δ ppm 7.49 (s, 1H) 7.46-7.44 (m, 3H) 4.63-4.57 (m, 1H) 3.94 (t, J=14 Hz, 2H) 3.46-3.42 (m, 1H) 1.57-1.53 (m, 3H) 1.25 (s, 9H) 0.60-0.45 (m, 4H).

### Step 4.

To a solution of N-[(1R)-1-[3-[2-(cyclopropoxy)-1,1-difluoro-ethyl]phenyl]ethyl]-2-methyl-propane-2-sulfinamide (60 mg, 173 μmol) in MeOH (1 mL) was added a 4M solution of HCl in MeOH (86.8 μL, 347 μmol). The mixture was stirred at 25° C. for 2 h. A saturated solution of NaOH in MeOH was added until pH ˜ 7 and the mixture was concentrated under reduced pressure. The residue was diluted in a 10:1 DCM/MeOH mixture, stirred for 30 min, then filtered and concentrated to give (1R)-1-[3-[2-(cyclopropoxy)-1,1-difluoro-ethyl]phenyl] ethanamine (41.9 mg, crude). LCMS (ESI): m/z: [M+H] calculated for C13H₁₈F₂NO: 242.1. found: 242.1.

### Step 5.

To a solution of (1R)-1-[3-[2-(cyclopropoxy)-1,1-difluoro-ethyl]phenyl]ethanamine (41.9 mg, 174 μmol) and (2,4-dichloro-5,7-dihydropyrrolo[3,4-d]pyrimidin-6-yl)-morpholino-methanone (52.6 mg, 174 μmol) in t-BuOH (1.5 mL) was added DIEA (151 μL, 868 μmol). The mixture was stirred at 80° C. for 16 h. The mixture was filtered and concentrated under reduced pressure and the crude residue was purified by prep-HPLC to give [2-chloro-4-[[(1R)-1-[3-[2-(cyclopropoxy)-1,1-difluoro-ethyl]phenyl]ethyl]amino]-5,7-dihydropyrrolo[3,4-d]pyrimidin-6-yl]-morpholino-methanone (7.1 mg, 8% yield). LCMS (ESI): m/z: [M+H] calculated for C₂₄H₂₉ClF₂N₅O₃: 508.1. found 508.2; ¹H NMR (400 MHz, METHANOL-d₄) δ ppm 7.56 (s, 1H) 7.52 (d, J=8 Hz, 1H) 7.45-7.38 (m, 2H) 5.43-5.39 (m, 1H) 4.63 (br s, 2H) 4.57 (br s, 2H) 3.96-3.89 (m, 2H) 3.74-3.71 (m, 4H) 3.37-3.35 (m, 5H) 1.59 (d, J=8 Hz, 3H) 0.41-0.39 (m, 4H).

### Example 532. Synthesis of [2-chloro-4-[1-(5-phenyl-2-thienyl)ethylamino]-5,7-dihydropyrrolo[3,4-d]pyrimidin-6-yl]-morpholino-methanone

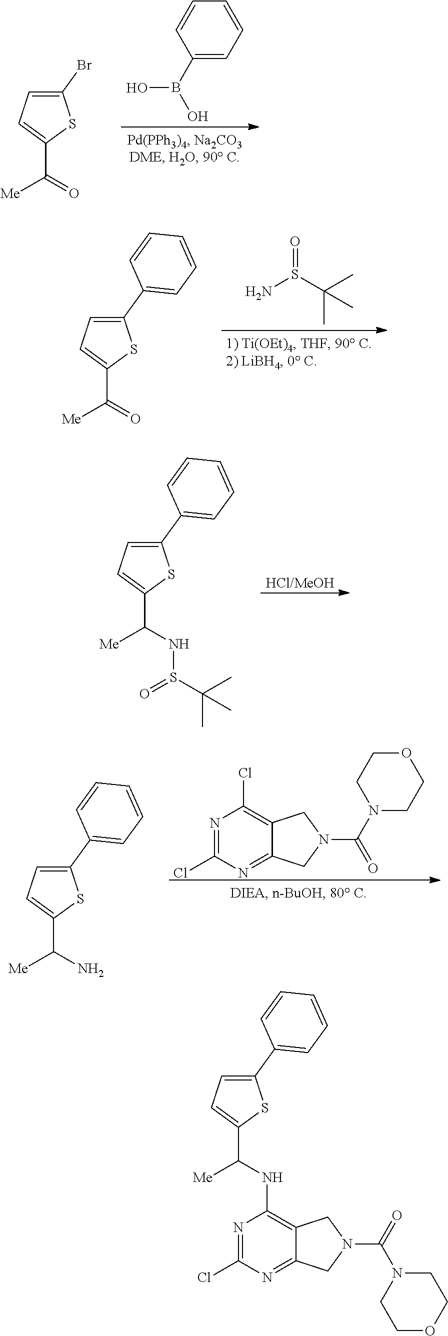

### Step 1.

To a solution of 1-(5-bromo-2-thienyl)ethanone (1 g, 4.88 mmol) and phenylboronic acid (713.48 mg, 5.85 mmol) in DME (10 mL) and H₂O (2 mL) was added Na₂CO₃ (1.55 g, 14.63 mmol) and Pd(PPh₃)₄(1.13 g, 975.27 μmol). The mixture was stirred at 90° C. for 16h under N₂, then diluted with water and extracted with EtOAc. The combined organic phase was washed with brine, dried over anhydrous Na₂SO₄, filtered and concentrated under reduced pressure. The residue was purified by column chromatography to give 1-(5-phenyl-2-thienyl)ethanone (750 mg, 76.04% yield). LCMS (ESI): m/z: [M+H] calculated for C12H_{II}OS: 203.5. found 203.2.

### Step 2.

To a solution of 1-(5-phenyl-2-thienyl)ethanone (750 mg, 3.71 mmol) in THF (7.5 mL) was added 2-methylpropane-2-sulfinamide (898.80 mg, 7.42 mmol) and Ti(OEt)₄ (2.54 g, 11.12 mmol, 2.31 mL). The mixture was stirred at 90° C. for 16h. Then MeOH (118.81 mg, 3.71 mmol, 150.05 μL) and LiBH₄ (242.31 mg, 11.12 mmol) were added to the mixture at 0° C. and stirred for 1h. The reaction mixture was then diluted with water (10.0 mL) and extracted with EtOAc. The combined organic phase was washed with brine, dried over anhydrous Na₂SO₄, filtered and concentrated under reduced pressure. The residue was purified by column chromatography to give 2-methyl-N-[1-(5-phenyl-2-thienyl)ethyl]propane-2-sulfinamide (385 mg, 33.71% yield). LCMS (ESI): m/z: [M+H] calculated for C₁₋₆H₂₂NOS₂: 308.1. found 308.2.

### Step 3.

A solution of 2-methyl-N-[1-(5-phenyl-2-thienyl)ethyl]propane-2-sulfinamide (380 mg, 1.24 mmol) in HCl/MeOH (4 M, 4 mL) was stirred at rt for 2 h. To the mixture was added NaOH/MeOH to PH=8. The mixture was concentrated under reduced pressure to give 1-(5-phenyl-2-thienyl)ethan amine (250 mg, 99.50% yield). LCMS (ESI): m/z: [M−NH2] calculated for C₁₂H₁₁S: 187.06. found 187.1.

### Step 4.

To a solution of 1-(5-phenyl-2-thienyl)ethanamine (150 mg, 737.82 μmol) and (2,4-dichloro-5,7-dihydropyrrolo[3,4-d]pyrimidin-6-yl)-morpholino-methanone (246.03 mg, 811.60 μmol) in n-BuOH (1.5 mL) was added DIEA (286.07 mg, 2.21 mmol, 385.54 μL). The mixture was stirred at 80° C. for 16 h. The mixture was concentrated under reduced pressure and purified by prep-HPLC to give [2-chloro-4-[1-(5-phenyl-2-thienyl)ethylamino]-5,7-dihydropyrrolo[3,4-d]pyrimidin-6-yl]-morpholino-methanone (50 mg, 14.42% yield). LCMS (ESI): m/z: [M+H] calculated for C₂₃H₂₅ClN₅O₂S: 470.1. found 470.1; ¹H NMR (400 MHz, DMSO-d6) δ ppm 8.27-8.29 (d, J=8.00, 1H) 7.58-7.60 (d, J=8.40, 2H) 7.35-7.40 (m, 3H) 7.26-7.30 (t, J=7.20, 1H) 7.03-7.04 (d, J=3.20 Hz, 1H) 5.54-5.57 (m, 1H) 4.52 (s, 4H) 3.60-3.62 (t, J=4.40, 4H) 3.22-3.24 (t, J=4.40, 4H) 1.60-1.62 (d, J=7.20, 3H).

### Example 533. Synthesis of [4-[1-[3-amino-4-fluoro-5-(trifluoromethyl)phenyl]ethylamino]-2-chloro-5,7-dihydropyrrolo[3,4-d]pyrimidin-6-yl]-morpholino-methanone

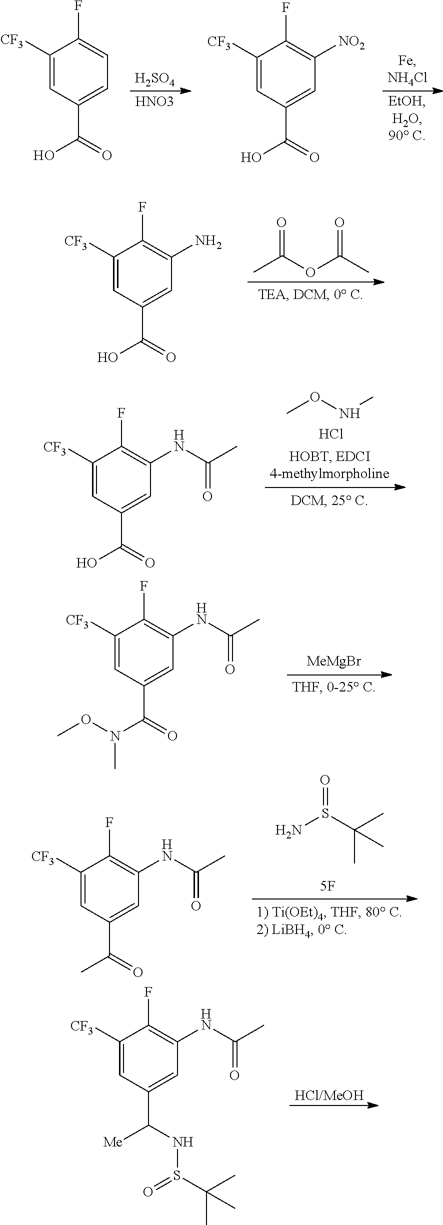

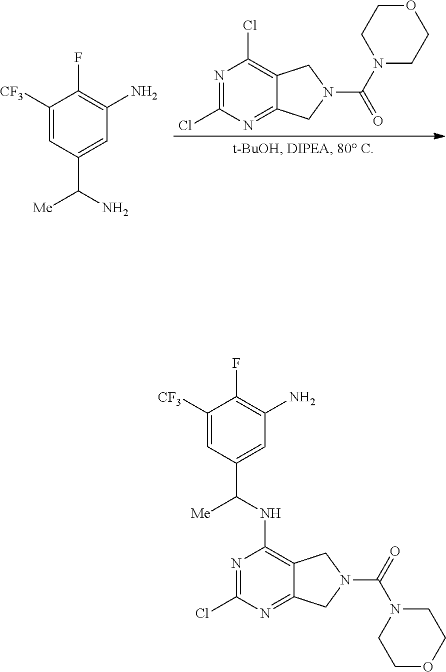 [SMILES: CC(N)c1cc(N)c(F)c(C(F)(F)F)c1 ; O=C(N1CCOCC1)N1Cc2nc(Cl)nc(Cl)c2C1 ; CC(Nc1nc(Cl)nc2c1CN(C(=O)N1CCOCC1)C2)c1cc(N)c(F)c(C(F)(F)F)c1]

### Step 1.

To a mixture of 4-fluoro-3-(trifluoromethyl)benzoic acid (3 g, 14.42 mmol) in HNO₃ (25 mL) was added H₂SO₄ (6.90 g, 70.35 mmol, 3.75 mL) at 0° C. The mixture was stirred at 75° C. for 5 h. The reaction mixture was quenched with ice water (40 mL) and extracted with EtOAc. The combined organic layers was dried over Na₂SO₄, filtered and concentrated under reduced pressure to give 4-fluoro-3-nitro-5-(trifluoromethyl)benzoic acid (2.5 g, 68.52% yield). ¹H NMR (400 MHz, DMSO-d₆) δ=8.78 (dd, J=4.5, 1H), 8.47 (dd, J=3.25, 1H).

To a solution of 4-fluoro-3-nitro-5-(trifluoromethyl)benzoic acid (2.5 g, 9.88 mmol) in EtOH (10 mL) and H₂O (5 mL) was added NH₄Cl (264.17 mg, 4.94 mmol) and Fe (1.65 g, 29.63 mmol) at rt. The mixture was stirred at rt for 5 h. The reaction mixture was quenched with ice water (40 mL) and extracted with EtOAc. The combined organic layers were dried over Na₂SO₄, filtered and concentrated under reduced pressure to give 3-amino-4-fluoro-5-(trifluoromethyl)benzoic acid (2.2 g, 99.83% yield). LCMS (ESI): m/z: [M+H] calculated for C₈H₆F₄NO₂: 224.0. found 224.0.

### Step 3.

To a solution of 3-amino-4-fluoro-5-(trifluoromethyl)benzoic acid (2.2 g, 9.86 mmol) in DCM (10 mL) was added TEA (997.73 mg, 9.86 mmol, 1.37 mL) and acetyl acetate (3.52 g, 34.51 mmol, 3.23 mL). The mixture was stirred at rt for 3 h. To the reaction mixture was added aq. NaHCO₃ (5 mL). The mixture was filtered, and the filtrate was extracted with DCM. The combined organic layer was dried over Na₂SO₄ and filtered. The solvent was concentrated under reduced pressure and the residue was purified by column chromatography to give 3-acetamido-4-fluoro-5-(trifluoromethyl)benzoic acid (2.6 g, 99.45% yield). ¹H NMR (CHLOROFORM-d, 400 MHz) δ ppm=8.85 (s, J=6.0 Hz 1H), 8.08 (d, J=4.8 Hz, 1H), 2.19 (m, 3H)

### Step 4.

To a solution of 3-acetamido-4-fluoro-5-(trifluoromethyl)benzoic acid (2.6 g, 9.81 mmol) in DCM (20 mL) was added HOBt (2.65 g, 19.61 mmol), 4-methylmorpholine (2.98 g, 29.42 mmol, 3.23 mL), N-methoxymethanamine; hydrochloride (1.91 g, 14.27 mmol) and EDCI (3.76 g, 19.61 mmol). The mixture was stirred at rt for 5 h. The reaction mixture was concentrated under reduced pressure and the residue was diluted with H₂O (20 mL) and extracted with EtOAc. The combined organic layers were washed with brine, dried over Na₂SO₄, filtered and concentrated under reduced pressure to give a residue. The residue was purified by column chromatography to give 3-acetamido-4-fluoro-N-methoxy-N-methyl-5-(trifluoromethyl)benzamide (1.3 g, 43.01% yield). LCMS (ESI): m/z: [M+H] calculated for C₁₂H₁₃F₄N₂O₃: 309.1. found 309.1.

### Step 5.

To a solution of 3-acetamido-4-fluoro-N-methoxy-N-methyl-5-(trifluoromethyl)benzamide (1 g, 3.24 mmol) in THF (10 mL) was added LiHMDS (1 M, 3.24 mL) at 0° C. and stirred for 30 min. Then MeMgBr (3 M, 1.08 mL) was added to the mixture. The mixture was stirred at 0° C. for 3 h. The reaction was poured onto ice water (2 mL) and adjusted to pH=4 with HCl, then extracted with EtOAc. The combined organic phase was washed with brine, dried over anhydrous Na₂SO₄, filtered and concentrated in vacuo. The residue was purified by prep-TLC to give N-[5-acetyl-2-fluoro-3-(trifluoromethyl)phenyl]acetamide (700 mg, 81.98% yield).

### Step 6.

To a solution of N-[5-acetyl-2-fluoro-3-(trifluoromethyl)phenyl]acetamide (110 mg, 417.95 μmol) in THF (5 mL) was added Ti(OEt)4 (286.02 mg, 1.25 mmol, 260.02 μL) and 2-methylpropane-2-sulfinamide (101.31 mg, 835.90 μmol) at rt. The mixture was stirred at 80° C. for 4 h. After cooling to −4° C., to the mixture was added MeOH (1 mL) then LiBH4 (27.31 mg, 1.25 mmol) at 0° C. and stirred for 1 h. The reaction was poured slowly onto H2O (2 mL) and THF (2 mL), then filtered over celite. The filter cake was washed with THF and the filtrate was concentrated under reduced pressure and the residue was purified by prep-TLC to give N-[5-[1-(tert-butylsulfinylamino)ethyl]-2-fluoro-3-(trifluoromethyl)phenyl]acetamide (140 mg, 90.93% yield). LCMS (ESI): m/z: [M+H] calculated for C₁₅H₂₁F₄N₂O₂S: 369.1. found 369.1.

### Step 7.

To a solution of N-[5-[1-(tert-butylsulfinylamino)ethyl]-2-fluoro-3-(trifluoromethyl)phenyl]acetamide (140 mg, 380.03 μmol) in MeOH (1 mL) was added HCl/MeOH (4 M, 95.01 μL). The mixture was stirred at rt for 8 h. The mixture was concentrated under reduced pressure to give a residue. The residue was dissolve in MeOH (1 mL). To the solution was added aq. MeOH/NaOH until pH=8. The solution was filtered and concentrated under reduced pressure to give the residue. The residue was washed with DCM/MeOH=10/1, filtered, and the filtrate was concentrated under reduced pressure to give 5-(1-aminoethyl)-2-fluoro-3-(trifluoromethyl)aniline (50 mg, 59.22% yield). LCMS (ESI): m/z: [M+H] calculated for C₉H₁₁F₄N₂: 223.1. found 223.1.

### Step 8.

To a solution of 5-(1-aminoethyl)-2-fluoro-3-(trifluoromethyl)aniline 2 HCl (50 mg, 169.43 μmol) in n-BuOH (1 mL) was added DIEA (65.69 mg, 508.29 μmol, 88.53 μL) and (2,4-dichloro-5,7-dihydropyrrolo[3,4-d]pyrimidin-6-yl)-morpholino-methanone (56.50 mg, 186.37 μmol) at rt. The mixture was stirred at 80° C. for 2 h. The mixture was concentrated under reduced pressure and the residue was purified by prep-HPLC to give [4-[1-[3-amino-4-fluoro-5-(trifluoromethyl)phenyl]ethylamino]-2-chloro-5,7-dihydropyrrolo[3,4-d]pyrimidin-6-yl]-morpholino-methanone (60 mg, 72.44% yield). LCMS (ESI): m/z: [M+H] calculated for C₂₀H₂₂ClF₄N₆O₂: 489.1. found 489.1; ¹H NMR (400 MHz, METHANOL-d₄) δ=7.07 (d, J=7.9, 1H), 6.89 (d, J=4.0, 1H), 5.30 (d, J=6.5, 1H), 4.59 (d, J=14.4, 4H), 3.76-3.70 (m, 4H), 3.35 (d, J=4.4, 4H), 1.53 (d, J=7.0, 3H).

### Example 534. Synthesis of [2-chloro-4-[1-(3-cyclopropylphenyl)ethylamino]-5,7-dihydropyrrolo[3,4-d]pyrimidin-6-yl]-morpholino-methanone

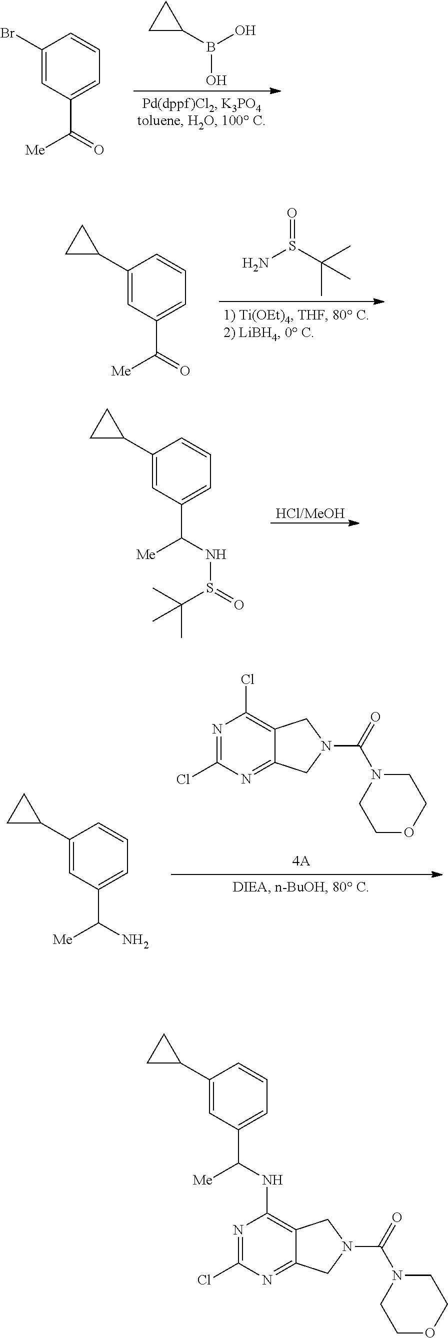

### Step 1.

To a solution of 1-(3-bromophenyl)ethanone (1 g, 5.02 mmol, 662.25 μL) in toluene (10 mL) was added cyclopropylboronic acid (863.10 mg, 10.05 mmol), Pd(dppf)Cl₂ (367.61 mg, 502.40 μmol), K₃PO₄ (3.20 g, 15.07 mmol), and H₂O (0.5 mL). The reaction mixture was stirred and heated to 100° C. under N₂ for 10 h. The reaction mixture was diluted with H₂O (10 mL) and extracted with EtOAc. The combined organic layers were washed with aqueous NaCl, dried over Na₂SO₄, filtered and concentrated under reduced pressure to give a residue. The residue was purified by column chromatography to give 1-(3-cyclopropylphenyl)ethanone (580 mg, 72.06% yield). LCMS (ESI): m/z: [M+H] calculated for C₁₁H₁₃O: 161.1. found 161.0; ¹H NMR (400 MHz, CHLOROFORM-d) δ ppm 7.73 (dt, J=7.6, 1.4 Hz, 1H) 7.68 (t, J=1.8 Hz, 1H) 7.35 (t, J=7.6 Hz, 1H) 7.27 (dt, J=7.6, 1.4 Hz, 1H) 2.60 (s, 3H) 2.00-1.93 (m, 1H) 1.04-0.99 (m, 2H) 0.77-0.73 (m, 2H).

### Step 2.

A mixture of 1-(3-cyclopropylphenyl)ethanone (580 mg, 3.62 mmol), 2-methylpropane-2-sulfinamide (877.54 mg, 7.24 mmol) in THF (10 mL) was added Ti(OEt)₄ (2.48 g, 10.86 mmol, 2.25 mL) at rt. The mixture was stirred at 90° C. for 10 h. After cooling to 0° C., MeOH (116.00 mg, 3.62 mmol, 146.50 μL) and LiBH₄ (78.86 mg, 3.62 mmol) were added to the mixture at 0° C. and stirred for 1 h. The reaction mixture was diluted with H₂O (10 mL) and filtered. The filtrate was extracted with EtOAc. The combined organic layers were washed with aqueous NaCl, dried over Na₂SO₄, filtered and concentrated under reduced pressure to give a residue. The residue was purified by column chromatography to give N-[1-(3-cyclopropylphenyl)ethyl]-2-methyl-propane-2-sulfinamide (380 mg, 39.55% yield). LCMS (ESI): m/z: [M+H] calculated for C₁₅H₂₄NOS: 266.2. found 266.1; ¹H NMR (400 MHz, METHANOL-d₄) δ ppm 7.22-7.18 (m, 1H) 7.15-7.11 (m, 2H) 6.96-6.94 (m, 1H) 4.42 (q, J=6.8 Hz, 1H) 1.94-1.87 (m, 1H) 1.48 (d, J=6.8 Hz, 3H) 1.22 (s, 9H) 0.97-0.92 (m, 2H) 0.70-0.66 (m, 2H).

### Step 3.

A solution of N-[1-(3-cyclopropylphenyl)ethyl]-2-methyl-propane-2-sulfinamide (380 mg, 1.43 mmol) in HCl/MeOH (4 M, 10 mL) was stirred at rt for 0.5 h. The reaction mixture was concentrated under reduced pressure to give 1-(3-cyclopropylphenyl) ethanamine HCl (280 mg, 99.04% yield).

### Step 4.

To a solution of (2,4-dichloro-5H-pyrrolo[3,4-d]pyrimidin-6(7H)-yl)(morpholino)methanone (150 mg, 494.81 μmol) in n-BuOH (2 mL) was added DIEA (319.76 mg, 2.47 mmol, 430.94 μL) and 1-(3-cyclopropylphenyl)ethanamine HCl (117.39 mg, 593.78 μmol). The mixture was stirred at 80° C. for 12 h. The reaction mixture was concentrated under reduced pressure to give a residue. The residue was purified by prep-HPLC to give [2-chloro-4-[1-(3-cyclopropylphenyl)ethylamino]-5,7-dihydropyrrolo[3,4-d]pyrimidin-6-yl]-morpholino-methanone (74.3 mg, 34.75% yield). LCMS (ESI): m/z: [M+H] calculated for C₂₂H₂₇ClN₅O₂: 428.2. found 428.2; H NMR (400 MHz, METHANOL-d4) δ ppm 7.20-7.12 (m, 3H) 6.93 (dt, J=7.2, 1.6 Hz, 1H) 5.36 (q, J=6.8 Hz, 1H) 4.59-4.55 (m, 4H) 3.72 (t, J=4.8 Hz, 4H) 3.35 (t, J=4.6 Hz, 4H) 1.92-1.86 (m, 1H) 1.54 (d, J=6.8 Hz, 3H) 0.96-0.92 (m, 2H) 0.69-0.65 (m, 2H).

### Example 535. Synthesis of 3-[1-[[2-chloro-6-(morpholine-4-carbonyl)-5,7-dihydropyrrolo[3,4-d]pyrimidin-4-yl]amino]ethyl]benzenesulfonamide

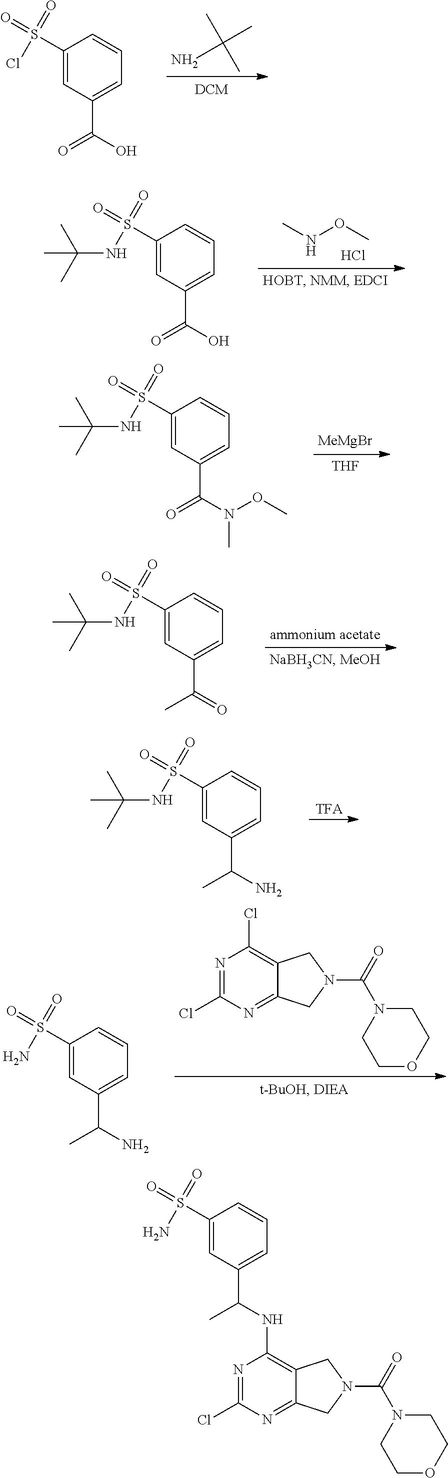

### Step 1.

To a solution of 3-chlorosulfonylbenzoic acid (1 g, 4.53 mmol) in DCM (10 mL) was added a solution of 2-methylpropan-2-amine (1.16 g, 15.86 mmol, 1.67 mL). The mixture was stirred at 0° C. for 0.5 h and at rt for 2 h. A white solid was formed during the reaction. The solid was filtered and washed with 10 mL of DCM. The solid was poured onto 20 mL of H₂O and 5 N aqueous HCl was added slowly (while stirring) until pH-5. The mixture was stirred at rt for 30 min. The mixture was filtered, washed with water and dried under vacuum to give 3-(tert-butylsulfamoyl) benzoic acid (0.9 g, 77.17% yield). ¹H NMR (400 MHz, DMSO-d₆) δ ppm 8.37 (t, J=1.6 Hz, 1H) 8.13 (dt, J=7.6, 1.4 Hz, 1H) 8.05 (dt, J=7.6, 1.4 Hz, 1H) 7.70 (t, J=7.8 Hz, 2H) 1.09 (s, 9H).

### Step 2.

To a solution of 3-(tert-butylsulfamoyl)benzoic acid (0.6 g, 2.33 mmol) in DCM (15 mL) was added HOBt (630.17 mg, 4.66 mmol) and N-methoxymethanamine; hydrochloride (409.43 mg, 4.20 mmol), followed by EDCI (894.04 mg, 4.66 mmol) and 4-methylmorpholine (707.58 mg, 7.00 mmol, 769.11 μL). The mixture was stirred at rt for 12 h. The reaction mixture was washed with H₂O and aqueous NaCl, dried over Na₂SO₄, filtered and concentrated under reduced pressure to give 3-(tert-butylsulfamoyl)-N-methoxy-N-methyl-benzamide (630 mg, 89.95% yield). LCMS (ESI): m/z: [M+H] calculated for C13H₂1N₂O4S: 301.1. found 301.1; ¹H NMR (400 MHz, CHLOROFORM-d) δ ppm 8.24 (t, J=1.4 Hz, 1H) 8.00-7.98 (m, 1H) 7.89-7.87 (m, 1H) 7.56 (t, J=7.8 Hz, 1H) 4.55 (s, 1H) 3.54 (s, 3H) 3.39 (s, 3H) 1.25 (s, 9H).

### Step 3.

To a solution of 3-(tert-butylsulfamoyl)-N-methoxy-N-methyl-benzamide (0.5 g, 1.66 mmol) in THF (10 mL) was added drop wise MeMgBr (3 M, 1.66 mL) at −78° C. The mixture was stirred at −78° C. for 20 min and then warmed to rt for 12 hr. The reaction mixture was quenched with aqueous NH₄Cl (5 mL) at −78° C. The mixture was then extracted with EtOAc. The organic layers were washed with brine and dried over Na₂SO₄, filtered and concentrated under reduced pressure to give 3-acetyl-N-tert-butyl-benzenesulfonamide (310 mg, 72.94% yield). ¹H NMR (400 MHz, CHLOROFORM-d) δ ppm 8.46 (t, J=1.6 Hz, 1H) 8.14-8.09 (m, 2H) 7.62 (t, J=7.8 Hz, 1H) 4.56 (s, 1H) 2.66 (s, 3H) 1.26 (s, 9H).

### Step 4.

To a solution of 3-acetyl-N-tert-butyl-benzenesulfonamide (260 mg, 1.02 mmol) in MeOH (3 mL) was added a solution of NaBH₃CN (95.99 mg, 1.53 mmol) and NH₄OAc (784.91 mg, 10.18 mmol) in MeOH (3 mL). The mixture was stirred at rt for 24 h. The reaction was acidified to pH-2 using 1 N HCl and then concentrated under reduced pressure. The residue was redissolved in water and then extracted with EtOAc. The aqueous layer was basified to pH-10 using saturated sodium hydroxide solution and then extracted with EtOAc, filtered and concentrated under reduced pressure to give 3-(1-aminoethyl)-N-tert-butyl-benzenesulfonamide (77 mg, 29.50% yield).

### Step 5.

A solution of 3-(1-aminoethyl)-N-tert-butyl-benzenesulfonamide (77 mg, 300.35 μmol) in TFA (2 mL) was stirred at tr for 5 h. The mixture was concentrated under reduced pressure to give 3-(1-aminoethyl)benzenesulfonamide TFA (90 mg, 95.34% yield). LCMS (ESI): m/z: [M+H] calculated for C₈H13N₂O₂S: 201.1. found 201.0.

### Step 6.

To a solution of 3-(1-aminoethyl)benzenesulfonamide TFA (90 mg, 286.37 μmol) and (2,4-dichloro-5,7-dihydropyrrolo[3,4-d]pyrimidin-6-yl)-morpholino-methanone (86.81 mg, 286.37 μmol) in n-BuOH (1 mL) was added DIEA (185.06 mg, 1.43 mmol, 249.40 μL). The mixture was stirred at 80° C. for 10 h. The reaction mixture was filtered and concentrated under reduced pressure to give a residue. The residue was purified by prep-HPLC to give 3-[1-[[2-chloro-6-(morpholine-4-carbonyl)-5,7-dihy dropyrrolo[3,4-d]pyrimidin-4-yl]amino]ethyl]benzenesulfonamide (36 mg, 26.92% yield). LCMS (ESI): m/z: [M+H] calculated for C₁₉H₂₄ClN₆O₄S: 467.1. found 467.0; H NMR (400 MHz, METHANOL-d₄) δ ppm 7.93 (s, 1H) 7.79 (d, J=8.0 Hz, 1H) 7.63 (d, J=7.6 Hz, 1H) 7.51 (t, J=7.8 Hz, 1H) 5.45 (q, J=6.8 Hz, 1H) 4.64-4.57 (m, 4H) 3.73 (t, J=4.8 Hz, 4H) 3.36 (t, J=4.6 Hz, 4H) 1.60 (d, J=6.8 Hz, 3H).

### Example 536. Synthesis of [2-chloro-4-[[(1R)-1-[3-(1,1-difluoro-2-methoxy-ethyl)phenyl]ethyl] amino]-5,7-dihydropyrrolo[3,4-d]pyrimidin-6-yl]-(4-methoxytetrahydropyran-4-yl)methanone

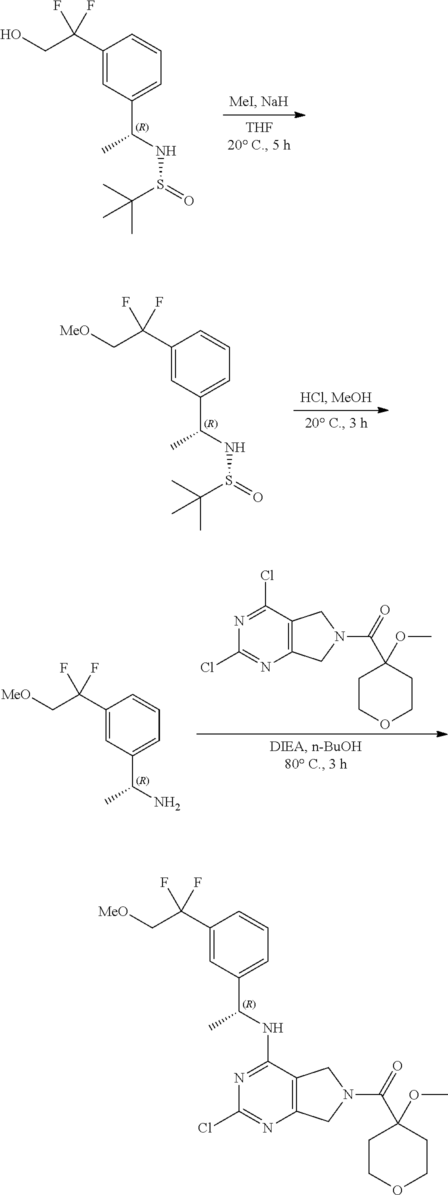

### Step 1.

A mixture of N-[(1R)-1-[3-(1,1-difluoro-2-hydroxy-ethyl)phenyl]ethyl]-2-methyl-propane-2-sulfinamide (500 mg, 1.64 mmol), NaH (65.49 mg, 1.64 mmol) in THF (10 mL) was added Mel (464.79 mg, 3.27 mmol, 203.85 μL). The mixture was stirred at rt for 5 h under N₂. The reaction mixture was quenched with 20 mL of water at 0° C., and then extracted with EtOAc. The combined organic layers were washed with 30 mL of brine, dried over anhydrous Na₂SO₄, filtered and concentrated under reduced pressure to give a residue. The residue was purified by column chromatography to give N-[(1R)-1-[3-(1,1-difluoro-2-methoxy-ethyl)phenyl]ethyl]-2-methyl-propane-2-sulfinamide (200 mg, 38.24% yield). LCMS (ESI): m/z: [M+H] calculated for C₁₅H₂₄F₂NO₂S:320.1. found 320.1; H NMR (400 MHz, CDCl₃) δ ppm=7.51-7.41 (m, 4H), 4.60 (qd, J=6.4, 2.8 Hz, 1H), 3.82 (t, J=13.2 Hz, 2H), 3.45 (s, 3H), 3.44 (br s, 1H), 1.54 (d, J=6.4 Hz, 3H), 1.25 (s, 9H).

### Step 2.

A mixture of N-[(1R)-1-[3-(1,1-difluoro-2-methoxy-ethyl)phenyl]ethyl]-2-methyl-propane-2-sulfinamide (200 mg, 626.16 μmol) in MeOH (5 mL) was added HCl/MeOH (4 M, 313.08 μL). The mixture was stirred at rt for 3 h. The reaction mixture was neutralized to pH=7 with NaOH in methanol and filtered. The filtrate was concentrated under reduced pressure to give a residue. The residue was washed with dichloromethane:methanol (5:1). The organic phases were combined and concentrated under reduced pressure to give (1R)-1-[3-(1,1-difluoro-2-methoxy-ethyl)phenyl]ethanamine (120 mg, 89.04% yield). LCMS (ESI): m/z: [M+H] calculated for C₁₁H₁₆F₂NO:216.1. found 216.0; ¹H NMR (400 MHz, DMSO-d₆) δ ppm=7.70 (s, 1H), 7.68-7.62 (m, 1H), 7.57-7.50 (m, 2H), 4.40 (q, J=6.8 Hz, 1H), 3.92 (t, J=13.6 Hz, 2H), 3.34 (s, 3H), 1.47 (d, J=6.8 Hz, 3H).

### Step 3.

A mixture of (1R)-1-[3-(1,1-difluoro-2-methoxy-ethyl)phenyl]ethanamine (60 mg, 278.76 μmol), (2,4-dichloro-5,7-dihydropyrrolo[3,4-d]pyrimidin-6-yl)-(4-methoxytetrahydro-pyran-4-yl)methanone (92.60 mg, 278.76 μmol) and DIEA (108.08 mg, 836.28 μmol, 145.66 μL) in n-BuOH (2 mL) was stirred at 80° C. for 3 h. The reaction mixture was filtered. The filtrate was purified by prep-HPLC to give [2-chloro-4-[[(1R)-1-[3-(1,1-difluoro-2-methoxy-ethyl)phenyl]ethyl]amino]-5,7-dihydropyrrolo[3,4-d]pyrimidin-6-yl]-(4-methoxytetrahydropyran-4-yl)methanone (25 mg, 16.06% yield). LCMS (ESI): m/z: [M+H] calculated for C₂₄H₃₀ClF₂N₄O₄:511.2. found 511.2; ¹H NMR (400 MHz, DMSO-d₆) δ ppm=8.37-8.25 (m, 1H), 7.59 (s, 1H), 7.56-7.49 (m, 1H), 7.49-7.38 (m, 2H), 5.37-5.28 (m, 1H), 4.87-4.75 (m, 2H), 4.59-4.47 (m, 2H), 3.89 (t, J=13.8 Hz, 2H), 3.74-3.54 (m, 4H), 3.17 and 3.13 (s, 3H), 2.02-1.81 (m, 4H), 1.50 (t, J=6.8 Hz, 3H); ¹⁹F NMR (376 MHz, DMSO-d₆) δ ppm=−101.02 (t, J=13.8 Hz), −101.06 (t, J=13.8 Hz).

### Example 540. Synthesis of N-[(1R)-1-[3-(difluoromethyl)-2-fluorophenyl]ethyl]-2-(fluoromethyl)-6-(morpholine-4-carbonyl)-5H,6H,7H-pyrrolo[3,4-d]pyrimidin-4-amine

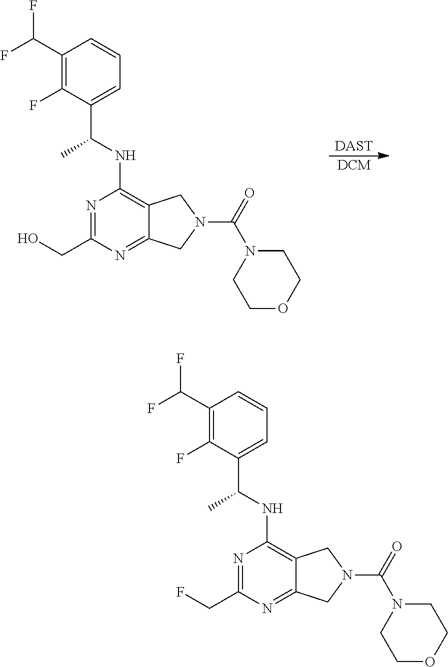

### Step 1.

To a solution of (4-{[(1R)-1-[3-(difluoromethyl)-2-fluorophenyl]ethyl]amino}-6-(morpholine-4-carbonyl)-5H,6H,7H-pyrrolo[3,4-d]pyrimidin-2-yl)methanol (420 mg, 0.93 mmol) in DCM (12.6 mL) DAST (123 μL, 0.93 mol) was added at 0° C. and the mixture was stirred for 2 h at rt. The reaction mixture was quenched with sat. aq NaHCO₃ solution and extracted with DCM. The combined organic layers were dried over Na₂SO₄ and the solvent was removed under reduced pressure. The crude product was purified by prep-HPLC to give N-[(1R)-1-[3-(difluoromethyl)-2-fluorophenyl]ethyl]-2-(fluoromethyl)-6-(morpholine-4-carbonyl)-5H,6H,7H-pyrrolo[3,4-d]pyrimidin-4-amine (19 mg, yield=5%). LCMS (ESI): exact mass for C₂₁H₂₃F₄N₅O₂: 454.2; [M+H]+=454.0 found; ¹H NMR (300 MHz, DMSO-d₆) δ 7.91 (d, J=7.5 Hz, 1H), 7.64 (t, J=7.4 Hz, 1H), 7.50 (t, J=7.1 Hz, 1H), 7.29 (t, J=7.7 Hz, 1H), 7.23 (t, J=54.3 Hz, 1H), 5.72-5.54 (m, 1H), 5.15 (dd, J=47.0, 3.8 Hz, 2H), 4.74-4.48 (m, 4H), 3.64 (t, J=4.6 Hz, 4H), 3.26 (t, J=4.7 Hz, 4H), 1.51 (d, J=7.0 Hz, 3H).

### Biological Examples

### Bodipy-FL-GTP Association Assay.

This assay was used to examine the potency with which compounds inhibit the SOS1-mediated exchange of KRAS-4B:GDP to KRAS-4B:GTP in a defined biochemical setting. A low IC₅₀ value for a given compound is indicative of high potency of said compound in inhibiting the guanine nucleotide exchange factor (GEF) activity of SOS1 on KRAS-4B in this assay setting.

Reagents: BODIPY FL GTP (ThermoFisher Scientific, Cat. G12411); KRAS4-B (Cytoskeleton Inc., Cat. CSRS03); SOS1 (Cytoskeleton Inc., Cat. CS-GE02); 2×Assay Buffer: 40 mM Tris-HCl, pH 7.5; 100 mM NaCl; 20 mM MgCl₂; 0.1 mg/mL BSA; 0.02% NP-40

Assay Procedure: Test compounds were dissolved in DMSO to create 20 mM master stocks. The stocks were diluted in a 3× dilution series in 100% DMSO to achieve 100× compound stocks. A 1 μl spot of each test compound stock was delivered to two adjacent wells of a 96-well assay plate prior to running the assay. Reaction Mix preparation: The following were mixed in order at room temperature to obtain the “Reaction Mix” (5.75 mL 2× Exchange Buffer; 3.22 mL MilliQ ddH2O; 3 μL 5 mM BODIPY FL GTP; 230 μL 50 μM KRAS-4B; 9.203 mL Total volume. Reaction initiation: 80 μL of Reaction Mix was pipetted into each well of a half-area black 96-well plate (Corning, Cat. 3686) containing either a 1 μL spot of DMSO or a 1 μL spot test compound at the concentrations listed above. 20 μL of 1 μM SOS1 was then added to each well to initiate the reaction. For the no GEF control wells this was replaced with 1× exchange buffer. Kinetic measurement: The reaction was monitored in a SpectraMax M2 Microplate Reader (Molecular Devices) under the following protocol: 5 second rapid circular mixing before first read; 61 readings, 30 seconds apart; Assay temperature: 22° C.; Excitation wavelength: 485 nm; Emission wavelength: 513 nm. Data Analysis: The Vmax values for the SOS1-mediated BODIPY FL GTP exchange curves in the presence of test compounds were normalized to the most dilute test sample columns or DMSO only control wells to give the % Activity for each concentration of test compound. Plots of % Activity vs. the Log 10 of the compound concentration were fit by non-linear regression to a 4-parameter logistic model.

### Bodipy-FL-GTP Association Assay (uM)

Bodipy-FL-GTP Association Assay results are shown in Table 8 below. Potency Table Key: <1 μM+; 1-5 μM++; >5 μM+++.

TABLE 8

| | Bodipy-FL-GTP |
|---|---|
| Example # | Association Assay |
| Example 1. | + |
| Example 2. | ++ |
| Example 3. | + |
| Example 4. | + |
| Example 5. | + |
| Example 6. | ++ |
| Example 7. | ++ |
| Example 8. | +++ |
| Example 9. | +++ |
| Example 10. | ++ |
| Example 11. | ++ |
| Example 12. | + |
| Example 13. | ++ |
| Example 14. | + |
| Example 15. | ++ |
| Example 16. | + |
| Example 17. | + |
| Example 18. | + |
| Example 19. | ++ |
| Example 20. | + |
| Example 21. | + |

Blank = Not Determined

### Mode of Action Assay: Inhibition of SOS1 Nucleotide Exchange Activity

The purpose of this assay was to characterize the inhibitory activity of compounds on SOS1 nucleotide exchange of KRAS. Data was reported as IC₅₀ values based on the TR-FRET signal.

Note—the following protocol describes a procedure for monitoring the inhibition of SOS1 nucleotide exchange activity of wild-type KRAS in response to a compound of the invention. Other KRAS mutants and RAS isoforms maybe employed.

In assay buffer containing 20 mM HEPES, pH 7.5, 150 mM NaCl, 5 mM MgCl₂, 0.05% Tween-20, 0.1% BSA, 1 mM DTT, concentration series of test compounds were generated spanning 100 μM to 1.7 nM over eleven 3-fold serial dilutions in a 384-well assay plate at a volume of 20 μL. The purified tagless catalytic domain of SOS1 (residues 564-1049) was first diluted in assay buffer at a concentration of 100 nM, and then 20 μL of the SOS1 containing solution was directly dispensed into compound plates. The SOS1/compound mixture was incubated at room temperature with constant mixing on an orbital shaker for 20 minutes to allow the reaction to reach equilibrium. A KRAS mixture was prepared by diluting 66.7 nM avi-tagged KRAS (residue 1-169), 3.33 nM Streptavidin-Tb and 333 nM EDA-GTP-DY-647P1 in assay buffer. This mixture was prepared immediately before addition to the SOS1/compound mixture to prevent intrinsic nucleotide exchange. Then 5 μL of the pre-incubated SOS1/compound mixture and 7.5 μL of the KRAS mixture were added sequentially in a 384-well low volume black round bottom plate and incubated at room temperature with constant shaking for 30 minutes. Time-resolved fluorescence was measured on a PerkinElmer Envision plate reader. DMSO and 10 μM of compound (i) were used as negative and positive controls, respectively.

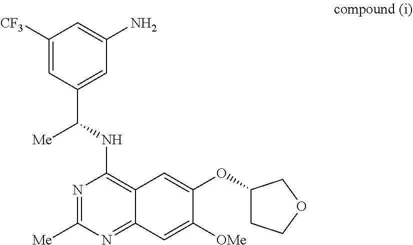 [SMILES: COc1cc2nc(C)nc(N[C@H](C)c3cc(N)cc(C(F)(F)F)c3)c2cc1O[C@H]1CCOC1]

Three replicates were performed for each compound. Data were normalized by the following: (Positive control−Sample signal)/(Positive control−negative control)*100. The data were fit using a four-parameter logistic fit.

SOS1 TR-FRET IC50 Assay results are shown in the Table 9 below: Table 6 Key: <1 μM+; >1 μM++.

TABLE 9

| Example # | TR-FRET IC50 |
|---|---|
| Example 22. | |
| Example 23. | |
| Example 24. | + |
| Example 25. | + |
| Example 26. | |
| Example 27. | + |
| Example 28. | |
| Example 29. | + |
| Example 30. | + |
| Example 31. | ++ |
| Example 32. | |
| Example 33. | |
| Example 34. | ++ |
| Example 35. | |
| Example 36. | ++ |
| Example 37. | + |
| Example 38. | + |
| Example 39. | |
| Example 40. | ++ |
| Example 41. | |
| Example 42. | ++ |
| Example 43. | ++ |
| Example 44. | ++ |
| Example 45. | + |
| Example 46. | ++ |
| Example 47. | + |
| Example 48., 166. | + |
| Example 49. | + |
| Example 50. | ++ |
| Example 51. | + |
| Example 52. | + |
| Example 53. | ++ |
| Example 54., 110. | ++ |
| Example 55. | + |
| Example 56. | + |
| Example 57. | ++ |
| Example 58. | + |
| Example 59. | + |
| Example 60. | ++ |
| Example 61. | ++ |
| Example 62. | ++ |
| Example 63. | ++ |
| Example 64. | ++ |
| Example 65. | + |
| Example 66. | ++ |
| Example 67. | ++ |
| Example 68. | + |
| Example 69. | ++ |
| Example 70. | ++ |
| Example 71. | + |
| Example 72. | + |
| Example 73. | + |
| Example 74. | + |
| Example 75. | + |
| Example 76. | ++ |
| Example 77. | + |
| Example 78. | + |
| Example 79. | |
| Example 80. | ++ |
| Example 81. | + |
| Example 82. | + |
| Example 83. | ++ |
| Example 84. | ++ |
| Example 85. | ++ |
| Example 86. | + |
| Example 87. | ++ |
| Example 88. | |
| Example 89. | + |
| Example 90. | ++ |
| Example 91. | ++ |
| Example 92. | ++ |
| Example 93. | + |
| Example 94. | + |
| Example 95. | ++ |
| Example 96. | ++ |
| Example 97. | ++ |
| Example 98. | |
| Example 99. | + |
| Example 100. | + |
| Example 101. | + |
| Example 102. | ++ |
| Example 103. | ++ |
| Example 104. | + |
| Example 105. | |
| Example 106. | |
| Example 107. | + |
| Example 108. | + |
| Example 109. | + |
| Example 111. | ++ |
| Example 112. | ++ |
| Example 113. | ++ |
| Example 114. | |
| Example 115. | ++ |
| Example 116. | + |
| Example 117. | |
| Example 118. | + |
| Example 119. | + |
| Example 120. | ++ |
| Example 121. | + |
| Example 122. | ++ |
| Example 123. | + |
| Example 124. | + |
| Example 125. | + |
| Example 126. | ++ |
| Example 127. | ++ |
| Example 128. | + |
| Example 129. | + |
| Example 130. | + |
| Example 131. | + |
| Example 132. | + |
| Example 133. | ++ |
| Example 134. | + |
| Example 135. | ++ |
| Example 136. | ++ |
| Example 137. | + |
| Example 138. | + |
| Example 139. | + |
| Example 140. | ++ |
| Example 141. | + |
| Example 142. | + |
| Example 143. | + |
| Example 144. | |
| Example 145. | + |
| Example 146. | + |
| Example 147. | ++ |
| Example 148. | + |
| Example 149. | |
| Example 150. | + |
| Example 151. | + |
| Example 152. | + |
| Example 153. | |
| Example 154. | + |
| Example 155. | |
| Example 156. | |
| Example 157. | |
| Example 158. | |
| Example 159. | + |
| Example 160. | + |
| Example 161. | |
| Example 162. | + |
| Example 163. | + |
| Example 164. | |
| Example 165. | + |
| Example 167. | + |
| Example 168. | + |
| Example 169. | + |
| Example 170. | + |
| Example 171. | + |
| Example 172. | + |
| Example 173. | ++ |
| Example 174. | ++ |
| Example 175. | ++ |
| Example 176. | + |
| Example 177. | + |
| Example 178. | + |
| Example 179. | + |
| Example 180. | + |
| Example 181. | + |
| Example 182. | + |
| Example 183. | + |
| Example 184. | + |
| Example 185. | + |
| Example 186. | |
| Example 187. | |
| Example 188. | ++ |
| Example 189. | ++ |
| Example 190. | ++ |
| Example 191. | ++ |
| Example 192. | ++ |
| Example 193. | ++ |
| Example 194. | ++ |
| Example 195. | ++ |
| Example 196. | ++ |
| Example 197. | ++ |
| Example 198. | ++ |
| Example 199. | ++ |
| Example 200. | ++ |
| Example 201. | ++ |
| Example 202. | ++ |
| Example 203. | ++ |
| Example 204. | + |
| Example 205. | ++ |
| Example 206. | ++ |
| Example 207. | ++ |
| Example 208. | ++ |
| Example 209. | ++ |
| Example 210. | ++ |
| Example 211. | ++ |
| Example 212. | ++ |
| Example 213. | ++ |
| Example 214. | ++ |
| Example 215. | ++ |
| Example 216. | ++ |
| Example 217. | ++ |
| Example 218. | ++ |
| Example 219. | + |
| Example 220. | ++ |
| Example 221. | + |
| Example 222. | ++ |
| Example 223. | + |
| Example 224. | ++ |
| Example 225. | + |
| Example 226. | ++ |
| Example 227. | + |
| Example 228. | + |
| Example 229. | ++ |
| Example 230. | ++ |
| Example 231. | ++ |
| Example 232. | ++ |
| Example 233. | ++ |
| Example 234. | ++ |
| Example 235. | + |
| Example 236. | ++ |
| Example 237. | |
| Example 238. | ++ |
| Example 239. | + |
| Example 240. | + |
| Example 241. | + |
| Example 242. | + |
| Example 243. | ++ |
| Example 244. | |
| Example 245. | ++ |
| Example 246. | ++ |
| Example 247. | ++ |
| Example 248. | ++ |
| Example 249. | ++ |
| Example 250. | ++ |
| Example 251. | |
| Example 252. | ++ |
| Example 253. | ++ |
| Example 254. | ++ |
| Example 255. | + |
| Example 256. | + |
| Example 257. | + |
| Example 258. | + |
| Example 259. | + |
| Example 260. | + |
| Example 261. | + |
| Example 262. | + |
| Example 263. | + |
| Example 264. | + |
| Example 265. | + |
| Example 266. | + |
| Example 267. | + |
| Example 268. | + |
| Example 269. | + |
| Example 270. | + |
| Example 271. | |
| Example 272. | + |
| Example 273. | + |
| Example 274. | + |
| Example 275. | + |
| Example 276. | + |
| Example 277. | |
| Example 278. | + |
| Example 279. | + |
| Example 280. | + |
| Example 281. | + |
| Example 282. | + |
| Example 283. | + |
| Example 284. | + |
| Example 285. | + |
| Example 286. | + |
| Example 287. | + |
| Example 288. | + |
| Example 289. | + |
| Example 290. | + |
| Example 291. | + |
| Example 292. | + |
| Example 293. | + |
| Example 294. | + |
| Example 295. | + |
| Example 296. | + |
| Example 297. | + |
| Example 298. | + |
| Example 299. | + |
| Example 300. | + |
| Example 301. | + |
| Example 302. | + |
| Example 303. | + |
| Example 304. | + |
| Example 305. | + |
| Example 306. | + |
| Example 307. | + |
| Example 308. | + |
| Example 309. | + |
| Example 310. | + |
| Example 311. | + |
| Example 312. | + |
| Example 313. | + |
| Example 314. | + |
| Example 315. | + |
| Example 316. | + |
| Example 317. | |
| Example 318. | + |
| Example 319. | + |
| Example 320. | + |
| Example 321. | + |
| Example 322. | + |
| Example 323. | + |
| Example 324. | + |
| Example 325. | + |
| Example 326. | + |
| Example 327. | + |
| Example 328. | + |
| Example 329. | + |
| Example 330. | + |
| Example 331. | + |
| Example 332. | + |
| Example 333. | + |
| Example 334. | + |
| Example 335. | + |
| Example 336. | + |
| Example 337. | + |
| Example 338. | + |
| Example 339. | + |
| Example 340. | + |
| Example 341. | + |
| Example 342. | + |
| Example 343. | + |
| Example 344. | + |
| Example 345. | + |
| Example 346. | + |
| Example 347. | + |
| Example 348. | + |
| Example 349. | + |
| Example 350. | + |
| Example 351. | |
| Example 352. | |
| Example 353. | + |
| Example 354. | + |
| Example 355. | + |
| Example 356. | + |
| Example 357. | + |
| Example 358. | + |
| Example 359. | + |
| Example 360. | + |
| Example 361. | |
| Example 362. | + |
| Example 363. | + |
| Example 364. | + |
| Example 365. | + |
| Example 366. | + |
| Example 367. | + |
| Example 368. | + |
| Example 369. | + |
| Example 370. | + |
| Example 371. | + |
| Example 372. | + |
| Example 373. | + |
| Example 374. | + |
| Example 375. | + |
| Example 376. | + |
| Example 377. | + |
| Example 378. | + |
| Example 379. | + |
| Example 380. | + |
| Example 381. | + |
| Example 382. | + |
| Example 383. | + |
| Example 384. | + |
| Example 385. | + |
| Example 386. | + |
| Example 387. | + |
| Example 388. | + |
| Example 389. | + |
| Example 390. | + |
| Example 391. | + |
| Example 392. | + |
| Example 393. | + |
| Example 394. | + |
| Example 395. | + |
| Example 396. | + |
| Example 397. | + |
| Example 398. | + |
| Example 399. | + |
| Example 400. | + |
| Example 401. | + |
| Example 402. | + |
| Example 403. | + |
| Example 404. | + |
| Example 405. | + |
| Example 406. | + |
| Example 407. | + |
| Example 408. | + |
| Example 409. | + |
| Example 410. | + |
| Example 411. | + |
| Example 412. | + |
| Example 413. | + |
| Example 414. | + |
| Example 415. | + |
| Example 416. | + |
| Example 417. | + |
| Example 418. | + |
| Example 419. | + |
| Example 420. | + |
| Example 421. | + |
| Example 422. | + |
| Example 423. | + |
| Example 424. | + |
| Example 425. | + |
| Example 426. | + |
| Example 427. | + |
| Example 428. | + |
| Example 429. | + |
| Example 430. | + |
| Example 431. | + |
| Example 432. | + |
| Example 433. | + |
| Example 434. | + |
| Example 435. | + |
| Example 436. | + |
| Example 437. | + |
| Example 438. | + |
| Example 439. | + |
| Example 440. | + |
| Example 441. | + |
| Example 442. | + |
| Example 443. | ++ |
| Example 444. | + |
| Example 445. | + |
| Example 446. | + |
| Example 447. | + |
| Example 448. | + |
| Example 449. | + |
| Example 450. | + |
| Example 451. | + |
| Example 452. | + |
| Example 453. | + |
| Example 454. | + |
| Example 455. | + |
| Example 456. | + |
| Example 457. | + |
| Example 458. | + |
| Example 459. | + |
| Example 460. | + |
| Example 461. | + |
| Example 462. | ++ |
| Example 463. | + |
| Example 464. | + |
| Example 465. | ++ |
| Example 466. | + |
| Example 467. | + |
| Example 468. | + |
| Example 469. | + |
| Example 470. | + |
| Example 471. | + |
| Example 472. | ++ |
| Example 473. | ++ |
| Example 474. | ++ |
| Example 475. | ++ |
| Example 476. | + |
| Example 477. | + |
| Example 478. | + |
| Example 479. | + |
| Example 480. | ++ |
| Example 481. | ++ |
| Example 482. | + |
| Example 483. | + |
| Example 484. | + |
| Example 485. | + |
| Example 486. | + |
| Example 487. | + |
| Example 488. | + |
| Example 489. | + |
| Example 490. | + |
| Example 491. | + |
| Example 492. | + |
| Example 493. | + |
| Example 494. | ++ |
| Example 495. | ++ |
| Example 496. | + |
| Example 497. | ++ |
| Example 498. | ++ |
| Example 499. | ++ |
| Example 500. | + |
| Example 501. | + |
| Example 502. | + |
| Example 503. | + |
| Example 504. | + |
| Example 505. | ++ |
| Example 506. | + |
| Example 507. | + |
| Example 508. | + |
| Example 509. | + |
| Example 510. | + |
| Example 511. | ++ |
| Example 512. | |
| Example 513. | |
| Example 514. | ++ |
| Example 515. | ++ |
| Example 516. | ++ |
| Example 517. | + |
| Example 518. | + |
| Example 519. | + |
| Example 520. | ++ |
| Example 521. | ++ |
| Example 522. | + |
| Example 523. | + |
| Example 524. | + |
| Example 525. | + |
| Example 526. | + |
| Example 527. | + |
| Example 528. | + |
| Example 529. | + |
| Example 530. | |
| Example 531. | + |
| Example 532. | ++ |
| Example 533. | ++ |
| Example 534. | ++ |
| Example 535. | ++ |
| Example 536. | + |
| Example 537. | + |
| Example 538. | + |
| Example 539. | + |
| Example 540. | + |

Blank = Not Determined

### Potency Assay: Measurement of the Binding Affinity of Compounds of the Invention to SOS1 Using Surface Plasmon Resonance (SPR)

The purpose of the SPR assay was to measure the direct binding of compounds to SOS1 catalytic domain (residues 564-1049) immobilized on a sensor chip. Data was reported as equilibrium dissociation constant (K_{d}) values.

Using a GE Biacore 8K SPR instrument, avi-tagged SOS1 catalytic domain protein was immobilized to a level of approximately 6000 response units (RU) on a streptavidin-coated SPR sensor chip in assay buffer containing 0.01 M HEPES, 0.15 M NaCl and 0.05% v/v Surfactant P20. In assay buffer containing 2% DMSO, concentration series of test compounds were generated spanning 5 μM to 4.9 nM over ten 2-fold dilutions. For each test compound, a separate 0 μM sample was generated for use during subsequent double reference subtraction. Serially for each test compound, individual dilution samples were flowed over the immobilized SOS1 protein at a flow rate of 50 μL/minute to monitor the association with SOS1. Dissociation of bound test compound from the SOS1 protein was immediately monitored by flowing assay buffer over the sensor surface and monitoring the decrease in binding signal back to the baseline level seen in the absence of compound. This was repeated for all compound dilutions in each series. The binding level response for each test compound concentration was noted immediately prior to the end of the association phase, and a secondary plot generated showing binding response level versus test compound concentration generated for each compound dilution series. This data was fitted to a model describing reversible equilibrium 1:1 binding between test compound and SOS1, yielding an estimate of the K_{d} value for the interaction.

SOS1 using Surface Plasmon Resonance (SPR) results are shown in the Table 10 below:

### Table 7 Key: ≤0.4 μM+; >0.4 μM++.

TABLE 10

| | SOS1 SPR |
|---|---|
| Example # | Equilibrium Kd |
| Example 22. | + |
| Example 23. | ++ |
| Example 24. | + |
| Example 25. | + |
| Example 26. | ++ |
| Example 27. | ++ |
| Example 28. | + |
| Example 29. | + |
| Example 30. | + |
| Example 31. | + |
| Example 32. | + |
| Example 33. | |
| Example 34. | ++ |
| Example 35. | ++ |
| Example 36. | ++ |
| Example 37. | + |
| Example 38. | |
| Example 39. | + |
| Example 40. | ++ |
| Example 41. | + |
| Example 42. | ++ |
| Example 43. | ++ |
| Example 44. | ++ |
| Example 45. | ++ |
| Example 46. | ++ |
| Example 47. | + |
| Example 48., 166. | + |
| Example 49. | + |
| Example 50. | ++ |
| Example 51. | + |
| Example 52. | + |
| Example 53. | ++ |
| Example 54., 110. | |
| Example 55. | |
| Example 56. | |
| Example 57. | |
| Example 58. | |
| Example 59. | + |
| Example 60. | + |
| Example 61. | + |
| Example 62. | + |
| Example 63. | + |
| Example 64. | ++ |
| Example 65. | ++ |
| Example 66. | ++ |
| Example 67. | ++ |
| Example 68. | + |
| Example 69. | ++ |
| Example 70. | ++ |
| Example 71. | + |
| Example 72. | ++ |
| Example 73. | + |
| Example 74. | + |
| Example 75. | + |
| Example 76. | ++ |
| Example 77. | ++ |
| Example 78. | + |
| Example 79. | + |
| Example 80. | ++ |
| Example 81. | + |
| Example 82. | + |
| Example 83. | ++ |
| Example 84. | + |
| Example 85. | ++ |
| Example 86. | + |
| Example 87. | ++ |
| Example 88. | + |
| Example 89. | + |
| Example 90. | ++ |
| Example 91. | ++ |
| Example 92. | + |
| Example 93. | + |
| Example 94. | + |
| Example 95. | ++ |
| Example 96. | + |
| Example 97. | ++ |
| Example 98. | + |
| Example 99. | ++ |
| Example 100. | + |
| Example 101. | + |
| Example 102. | ++ |
| Example 103. | + |
| Example 104. | + |
| Example 105. | ++ |
| Example 106. | + |
| Example 107. | + |
| Example 108. | + |
| Example 109. | |
| Example 111. | |
| Example 112. | |
| Example 113. | |
| Example 114. | |
| Example 115. | + |
| Example 116. | + |
| Example 117. | ++ |
| Example 118. | ++ |
| Example 119. | + |
| Example 120. | ++ |
| Example 121. | + |
| Example 122. | ++ |
| Example 123. | + |
| Example 124. | + |
| Example 125. | + |
| Example 126. | ++ |
| Example 127. | + |
| Example 128. | + |
| Example 129. | + |
| Example 130. | + |
| Example 131. | + |
| Example 132. | + |
| Example 133. | ++ |
| Example 134. | + |
| Example 135. | ++ |
| Example 136. | ++ |
| Example 137. | + |
| Example 138. | + |
| Example 139. | + |
| Example 140. | + |
| Example 141. | + |
| Example 142. | + |
| Example 143. | + |
| Example 144. | + |
| Example 145. | + |
| Example 146. | + |
| Example 147. | + |
| Example 148. | + |
| Example 149. | + |
| Example 150. | ++ |
| Example 151. | |
| Example 152. | + |
| Example 153. | + |
| Example 154. | + |
| Example 155. | + |
| Example 156. | + |
| Example 157. | + |
| Example 158. | + |
| Example 159. | + |
| Example 160. | + |
| Example 161. | + |
| Example 162. | + |
| Example 163. | + |
| Example 164. | + |
| Example 165. | + |
| Example 167. | + |
| Example 168. | + |
| Example 169. | + |
| Example 170. | + |
| Example 171. | + |
| Example 172. | + |
| Example 173. | ++ |
| Example 174. | ++ |
| Example 175. | ++ |
| Example 176. | |
| Example 177. | |
| Example 178. | + |
| Example 179. | + |
| Example 180. | + |
| Example 181. | + |
| Example 182. | + |
| Example 183. | + |
| Example 184. | + |
| Example 185. | ++ |
| Example 186. | ++ |
| Example 187. | + |
| Example 188. | |
| Example 189. | |
| Example 190. | |
| Example 191. | |
| Example 192. | |
| Example 193. | + |
| Example 194. | ++ |
| Example 195. | |
| Example 196. | |
| Example 197. | |
| Example 198. | |
| Example 199. | |
| Example 200. | |
| Example 201. | |
| Example 202. | |
| Example 203. | |
| Example 204. | |
| Example 205. | + |
| Example 206. | |
| Example 207. | |
| Example 208. | |
| Example 209. | |
| Example 210. | |
| Example 211. | |
| Example 212. | ++ |
| Example 213. | |
| Example 214. | |
| Example 215. | |
| Example 216. | |
| Example 217. | |
| Example 218. | |
| Example 219. | + |
| Example 220. | |
| Example 221. | ++ |
| Example 222. | ++ |
| Example 223. | + |
| Example 224. | |
| Example 225. | + |
| Example 226. | + |
| Example 227. | + |
| Example 228. | + |
| Example 229. | ++ |
| Example 230. | |
| Example 231. | ++ |
| Example 232. | ++ |
| Example 233. | ++ |
| Example 234. | ++ |
| Example 235. | ++ |
| Example 236. | ++ |
| Example 237. | ++ |
| Example 238. | ++ |
| Example 239. | + |
| Example 240. | + |
| Example 241. | + |
| Example 242. | |
| Example 243. | |
| Example 244. | |
| Example 245. | |
| Example 246. | ++ |
| Example 247. | ++ |
| Example 248. | ++ |
| Example 249. | |
| Example 250. | ++ |
| Example 251. | |
| Example 252. | |
| Example 253. | |
| Example 254. | ++ |
| Example 255. | + |
| Example 256. | + |
| Example 257. | + |
| Example 258. | |
| Example 259. | + |
| Example 260. | + |
| Example 261. | + |
| Example 262. | + |
| Example 263. | + |
| Example 264. | + |
| Example 265. | + |
| Example 266. | + |
| Example 267. | |
| Example 268. | |
| Example 269. | |
| Example 270. | + |
| Example 271. | |
| Example 272. | + |
| Example 273. | |
| Example 274. | + |
| Example 275. | ++ |
| Example 276. | ++ |
| Example 277. | |
| Example 278. | + |
| Example 279. | ++ |
| Example 280. | + |
| Example 281. | + |
| Example 282. | + |
| Example 283. | + |
| Example 284. | + |
| Example 285. | + |
| Example 286. | + |
| Example 287. | + |
| Example 288. | + |
| Example 289. | + |
| Example 290. | + |
| Example 291. | ++ |
| Example 292. | + |
| Example 293. | + |
| Example 294. | + |
| Example 295. | + |
| Example 296. | + |
| Example 297. | + |
| Example 298. | + |
| Example 299. | + |
| Example 300. | + |
| Example 301. | |
| Example 302. | + |
| Example 303. | + |
| Example 304. | + |
| Example 305. | + |
| Example 306. | + |
| Example 307. | + |
| Example 308. | |
| Example 309. | + |
| Example 310. | + |
| Example 311. | + |
| Example 312. | + |
| Example 313. | + |
| Example 314. | + |
| Example 315. | + |
| Example 316. | + |
| Example 317. | |
| Example 318. | + |
| Example 319. | + |
| Example 320. | + |
| Example 321. | + |
| Example 322. | + |
| Example 323. | + |
| Example 324. | + |
| Example 325. | + |
| Example 326. | + |
| Example 327. | + |
| Example 328. | + |
| Example 329. | + |
| Example 330. | + |
| Example 331. | + |
| Example 332. | ++ |
| Example 333. | + |
| Example 334. | + |
| Example 335. | + |
| Example 336. | + |
| Example 337. | + |
| Example 338. | + |
| Example 339. | + |
| Example 340. | + |
| Example 341. | + |
| Example 342. | + |
| Example 343. | + |
| Example 344. | + |
| Example 345. | + |
| Example 346. | + |
| Example 347. | + |
| Example 348. | + |
| Example 349. | + |
| Example 350. | + |
| Example 351. | + |
| Example 352. | + |
| Example 353. | + |
| Example 354. | + |
| Example 355. | + |
| Example 356. | + |
| Example 357. | + |
| Example 358. | + |
| Example 359. | + |
| Example 360. | + |
| Example 361. | |
| Example 362. | + |
| Example 363. | + |
| Example 364. | + |
| Example 365. | + |
| Example 366. | + |
| Example 367. | + |
| Example 368. | |
| Example 369. | + |
| Example 370. | + |
| Example 371. | |
| Example 372. | |
| Example 373. | |
| Example 374. | + |
| Example 375. | + |
| Example 376. | + |
| Example 377. | + |
| Example 378. | + |
| Example 379. | |
| Example 380. | + |
| Example 381. | + |
| Example 382. | + |
| Example 383. | + |
| Example 384. | + |
| Example 385. | + |
| Example 386. | + |
| Example 387. | + |
| Example 388. | + |
| Example 389. | + |
| Example 390. | + |
| Example 391. | + |
| Example 392. | + |
| Example 393. | + |
| Example 394. | + |
| Example 395. | + |
| Example 396. | + |
| Example 397. | + |
| Example 398. | + |
| Example 399. | + |
| Example 400. | + |
| Example 401. | + |
| Example 402. | + |
| Example 403. | + |
| Example 404. | + |
| Example 405. | + |
| Example 406. | + |
| Example 407. | + |
| Example 408. | + |
| Example 409. | + |
| Example 410. | + |
| Example 411. | + |
| Example 412. | + |
| Example 413. | + |
| Example 414. | + |
| Example 415. | + |
| Example 416. | + |
| Example 417. | + |
| Example 418. | + |
| Example 419. | + |
| Example 420. | + |
| Example 421. | + |
| Example 422. | + |
| Example 423. | + |
| Example 424. | + |
| Example 425. | + |
| Example 426. | ++ |
| Example 427. | + |
| Example 428. | + |
| Example 429. | + |
| Example 430. | + |
| Example 431. | + |
| Example 432. | + |
| Example 433. | + |
| Example 434. | + |
| Example 435. | + |
| Example 436. | + |
| Example 437. | + |
| Example 438. | + |
| Example 439. | + |
| Example 440. | + |
| Example 441. | |
| Example 442. | + |
| Example 443. | |
| Example 444. | + |
| Example 445. | ++ |
| Example 446. | + |
| Example 447. | + |
| Example 448. | + |
| Example 449. | + |
| Example 450. | + |
| Example 451. | + |
| Example 452. | + |
| Example 453. | + |
| Example 454. | + |
| Example 455. | + |
| Example 456. | + |
| Example 457. | + |
| Example 458. | + |
| Example 459. | + |
| Example 460. | + |
| Example 461. | + |
| Example 462. | |
| Example 463. | + |
| Example 464. | |
| Example 465. | |
| Example 466. | + |
| Example 467. | + |
| Example 468. | + |
| Example 469. | + |
| Example 470. | ++ |
| Example 471. | + |
| Example 472. | |
| Example 473. | |
| Example 474. | |
| Example 475. | |
| Example 476. | + |
| Example 477. | + |
| Example 478. | + |
| Example 479. | + |
| Example 480. | |
| Example 481. | |
| Example 482. | ++ |
| Example 483. | + |
| Example 484. | + |
| Example 485. | + |
| Example 486. | + |
| Example 487. | + |
| Example 488. | + |
| Example 489. | + |
| Example 490. | + |
| Example 491. | + |
| Example 492. | + |
| Example 493. | + |
| Example 494. | |
| Example 495. | |
| Example 496. | ++ |
| Example 497. | |
| Example 498. | |
| Example 499. | |
| Example 500. | |
| Example 501. | |
| Example 502. | |
| Example 503. | |
| Example 504. | ++ |
| Example 505. | ++ |
| Example 506. | |
| Example 507. | + |
| Example 508. | + |
| Example 509. | |
| Example 510. | + |
| Example 511. | |
| Example 512. | |
| Example 513. | |
| Example 514. | |
| Example 515. | |
| Example 516. | |
| Example 517. | |
| Example 518. | |
| Example 519. | |
| Example 520. | |
| Example 521. | |
| Example 522. | + |
| Example 523. | + |
| Example 524. | |
| Example 525. | + |
| Example 526. | + |
| Example 527. | + |
| Example 528. | ++ |
| Example 529. | + |
| Example 530. | + |
| Example 531. | |
| Example 532. | |
| Example 533. | |
| Example 534. | |
| Example 535. | |
| Example 536. | |
| Example 537. | + |
| Example 538. | + |
| Example 539. | ++ |
| Example 540. | + |

Blank = Not Determined

Potency Assay: pERK

The purpose of this assay is to measure the ability of test compounds to inhibit SOS1 function in cells. SOS1 activates RAS proteins by catalyzing the conversion of RAS GDP to RAS GTP in response to receptor tyrosine kinase activation. Activation of RAS induces a sequence of cellular signaling events that results in increased phosphorylation of ERK at Threonine 202 and Tyrosine 204 (pERK). The procedure described below measures the level of cellular pERK in response to test compounds in PC-9 cells (EGFR Ex19Del).

PC-9 cells were grown and maintained using media and procedures recommended by the ATCC. On the day prior to compound addition, cells were plated in 384-well cell culture plates (40 μL/well) and grown overnight in a 37° C., 5% CO₂ incubator. Test compounds were prepared in 10, 3-fold dilutions in DMSO, with a top concentration of 10 mM. On the day of the assay, 40 nL of test compound was added to each well of cell culture plate using an Echo550 liquid handler (LabCyte). Concentrations of test compound were tested in duplicate with highest test concentration being 10 μM. After compound addition, cells were incubated for 1 hour at 37° C., 5% CO₂. Following incubation, culture medium was removed and cells were washed once with phosphate buffered saline.

Cellular pERK level was determined using the AlphaLISA SureFire Ultra p-ERK1/2 Assay Kit (PerkinElmer). Cells were lysed in 25 μL lysis buffer, with shaking at 600 RPM at room temperature for 15 minutes. Lysate (10 μL) was transferred to a 384-well Opti-plate (PerkinElmer) and 5 μL acceptor mix was added. The plate was centrifuged at 1000 RPM for 1 minute, and incubated in the dark for 2 hours. Following this incubation, 5 μL of donor mix was added, the plate was sealed and centrifuged at 1000 RPM for 1 minute, and the mixture was incubated for 2 hours at room temperature. Signal was read on an Envision plate reader (PerkinElmer) using standard AlphaLISA settings. Analysis of raw data was carried out in Excel (Microsoft) and Prism (GraphPad). Signal was plotted vs. the decadal logarithm of compound concentration, and IC₅₀ was determined by fitting a 4-parameter sigmoidal concentration response model.

SOS1 pERK IC50 Assay results are shown in the Table 11 below. Table 8 Key: ≤1 μM+; >1 μM++.

TABLE 11

| | SOS1 pERK |
|---|---|
| Example # | IC50 |
| Example 22. | + |
| Example 23. | ++ |
| Example 24. | + |
| Example 25. | |
| Example 26. | |
| Example 27. | |
| Example 28. | |
| Example 29. | + |
| Example 30. | ++ |
| Example 31. | ++ |
| Example 32. | + |
| Example 33. | |
| Example 34. | + |
| Example 35. | ++ |
| Example 36. | ++ |
| Example 37. | + |
| Example 38. | + |
| Example 39. | + |
| Example 40. | ++ |
| Example 41. | + |
| Example 42. | |
| Example 43. | |
| Example 44. | |
| Example 45. | + |
| Example 46. | ++ |
| Example 47. | + |
| Example 48., 166. | ++ |
| Example 49. | + |
| Example 50. | |
| Example 51. | + |
| Example 52. | + |
| Example 53. | ++ |
| Example 54., 110. | ++ |
| Example 55. | ++ |
| Example 56. | ++ |
| Example 57. | ++ |
| Example 58. | + |
| Example 59. | + |
| Example 60. | + |
| Example 61. | + |
| Example 62. | + |
| Example 63. | |
| Example 64. | |
| Example 65. | + |
| Example 66. | |
| Example 67. | |
| Example 68. | + |
| Example 69. | |
| Example 70. | + |
| Example 71. | + |
| Example 72. | + |
| Example 73. | + |
| Example 74. | |
| Example 75. | + |
| Example 76. | |
| Example 77. | + |
| Example 78. | + |
| Example 79. | |
| Example 80. | |
| Example 81. | |
| Example 82. | + |
| Example 83. | + |
| Example 84. | + |
| Example 85. | |
| Example 86. | + |
| Example 87. | + |
| Example 88. | |
| Example 89. | |
| Example 90. | |
| Example 91. | + |
| Example 92. | ++ |
| Example 93. | + |
| Example 94. | + |
| Example 95. | |
| Example 96. | |
| Example 97. | ++ |
| Example 98. | |
| Example 99. | |
| Example 100. | |
| Example 101. | + |
| Example 102. | + |
| Example 103. | |
| Example 104. | + |
| Example 105. | |
| Example 106. | |
| Example 107. | + |
| Example 108. | + |
| Example 109. | ++ |
| Example 111. | ++ |
| Example 112. | ++ |
| Example 113. | |
| Example 114. | |
| Example 115. | + |
| Example 116. | |
| Example 117. | |
| Example 118. | + |
| Example 119. | ++ |
| Example 120. | ++ |
| Example 121. | |
| Example 122. | |
| Example 123. | + |
| Example 124. | |
| Example 125. | + |
| Example 126. | + |
| Example 127. | |
| Example 128. | + |
| Example 129. | + |
| Example 130. | ++ |
| Example 131. | + |
| Example 132. | + |
| Example 133. | |
| Example 134. | + |
| Example 135. | ++ |
| Example 136. | |
| Example 137. | + |
| Example 138. | + |
| Example 139. | |
| Example 140. | + |
| Example 141. | |
| Example 142. | + |
| Example 143. | |
| Example 144. | |
| Example 145. | + |
| Example 146. | + |
| Example 147. | |
| Example 148. | + |
| Example 149. | |
| Example 150. | |
| Example 151. | + |
| Example 152. | |
| Example 153. | |
| Example 154. | |
| Example 155. | |
| Example 156. | |
| Example 157. | |
| Example 158. | |
| Example 159. | |
| Example 160. | + |
| Example 161. | |
| Example 162. | |
| Example 163. | |
| Example 164. | |
| Example 165. | + |
| Example 167. | + |
| Example 168. | + |
| Example 169. | + |
| Example 170. | + |
| Example 171. | + |
| Example 172. | |
| Example 173. | |
| Example 174. | |
| Example 175. | |
| Example 176. | + |
| Example 177. | ++ |
| Example 178. | |
| Example 179. | + |
| Example 180. | + |
| Example 181. | + |
| Example 182. | |
| Example 183. | + |
| Example 184. | + |
| Example 185. | ++ |
| Example 186. | ++ |
| Example 187. | ++ |
| Example 188. | ++ |
| Example 189. | ++ |
| Example 190. | ++ |
| Example 191. | |
| Example 192. | |
| Example 193. | ++ |
| Example 194. | + |
| Example 195. | ++ |
| Example 196. | ++ |
| Example 197. | ++ |
| Example 198. | |
| Example 199. | ++ |
| Example 200. | ++ |
| Example 201. | ++ |
| Example 202. | ++ |
| Example 203. | ++ |
| Example 204. | ++ |
| Example 205. | + |
| Example 206. | ++ |
| Example 207. | |
| Example 208. | |
| Example 209. | ++ |
| Example 210. | ++ |
| Example 211. | ++ |
| Example 212. | + |
| Example 213. | ++ |
| Example 214. | ++ |
| Example 215. | ++ |
| Example 216. | ++ |
| Example 217. | |
| Example 218. | ++ |
| Example 219. | + |
| Example 220. | ++ |
| Example 221. | ++ |
| Example 222. | ++ |
| Example 223. | + |
| Example 224. | ++ |
| Example 225. | + |
| Example 226. | ++ |
| Example 227. | + |
| Example 228. | ++ |
| Example 229. | ++ |
| Example 230. | ++ |
| Example 231. | ++ |
| Example 232. | ++ |
| Example 233. | ++ |
| Example 234. | ++ |
| Example 235. | ++ |
| Example 236. | ++ |
| Example 237. | ++ |
| Example 238. | ++ |
| Example 239. | ++ |
| Example 240. | + |
| Example 241. | + |
| Example 242. | ++ |
| Example 243. | ++ |
| Example 244. | ++ |
| Example 245. | ++ |
| Example 246. | ++ |
| Example 247. | ++ |
| Example 248. | ++ |
| Example 249. | ++ |
| Example 250. | ++ |
| Example 251. | ++ |
| Example 252. | ++ |
| Example 253. | |
| Example 254. | ++ |
| Example 255. | + |
| Example 256. | + |
| Example 257. | ++ |
| Example 258. | + |
| Example 259. | + |
| Example 260. | + |
| Example 261. | + |
| Example 262. | + |
| Example 263. | + |
| Example 264. | ++ |
| Example 265. | + |
| Example 266. | ++ |
| Example 267. | + |
| Example 268. | ++ |
| Example 269. | ++ |
| Example 270. | ++ |
| Example 271. | ++ |
| Example 272. | + |
| Example 273. | + |
| Example 274. | + |
| Example 275. | ++ |
| Example 276. | ++ |
| Example 277. | ++ |
| Example 278. | + |
| Example 279. | + |
| Example 280. | + |
| Example 281. | + |
| Example 282. | + |
| Example 283. | + |
| Example 284. | + |
| Example 285. | + |
| Example 286. | + |
| Example 287. | + |
| Example 288. | + |
| Example 289. | + |
| Example 290. | + |
| Example 291. | + |
| Example 292. | + |
| Example 293. | + |
| Example 294. | + |
| Example 295. | + |
| Example 296. | + |
| Example 297. | + |
| Example 298. | ++ |
| Example 299. | + |
| Example 300. | + |
| Example 301. | + |
| Example 302. | + |
| Example 303. | + |
| Example 304. | ++ |
| Example 305. | ++ |
| Example 306. | + |
| Example 307. | + |
| Example 308. | ++ |
| Example 309. | + |
| Example 310. | + |
| Example 311. | + |
| Example 312. | + |
| Example 313. | + |
| Example 314. | ++ |
| Example 315. | + |
| Example 316. | + |
| Example 317. | + |
| Example 318. | ++ |
| Example 319. | + |
| Example 320. | + |
| Example 321. | + |
| Example 322. | + |
| Example 323. | + |
| Example 324. | + |
| Example 325. | + |
| Example 326. | + |
| Example 327. | + |
| Example 328. | + |
| Example 329. | + |
| Example 330. | + |
| Example 331. | + |
| Example 332. | ++ |
| Example 333. | + |
| Example 334. | + |
| Example 335. | + |
| Example 336. | ++ |
| Example 337. | ++ |
| Example 338. | + |
| Example 339. | + |
| Example 340. | + |
| Example 341. | + |
| Example 342. | + |
| Example 343. | + |
| Example 344. | + |
| Example 345. | |
| Example 346. | |
| Example 347. | |
| Example 348. | ++ |
| Example 349. | ++ |
| Example 350. | + |
| Example 351. | + |
| Example 352. | ++ |
| Example 353. | + |
| Example 354. | + |
| Example 355. | ++ |
| Example 356. | + |
| Example 357. | + |
| Example 358. | |
| Example 359. | + |
| Example 360. | ++ |
| Example 361. | |
| Example 362. | + |
| Example 363. | + |
| Example 364. | + |
| Example 365. | + |
| Example 366. | + |
| Example 367. | |
| Example 368. | + |
| Example 369. | |
| Example 370. | + |
| Example 371. | + |
| Example 372. | + |
| Example 373. | |
| Example 374. | + |
| Example 375. | + |
| Example 376. | + |
| Example 377. | + |
| Example 378. | ++ |
| Example 379. | + |
| Example 380. | ++ |
| Example 381. | + |
| Example 382. | ++ |
| Example 383. | + |
| Example 384. | + |
| Example 385. | + |
| Example 386. | + |
| Example 387. | + |
| Example 388. | + |
| Example 389. | ++ |
| Example 390. | + |
| Example 391. | + |
| Example 392. | ++ |
| Example 393. | ++ |
| Example 394. | + |
| Example 395. | + |
| Example 396. | + |
| Example 397. | + |
| Example 398. | ++ |
| Example 399. | ++ |
| Example 400. | + |
| Example 401. | + |
| Example 402. | + |
| Example 403. | + |
| Example 404. | + |
| Example 405. | ++ |
| Example 406. | + |
| Example 407. | + |
| Example 408. | + |
| Example 409. | + |
| Example 410. | + |
| Example 411. | + |
| Example 412. | + |
| Example 413. | + |
| Example 414. | + |
| Example 415. | + |
| Example 416. | + |
| Example 417. | + |
| Example 418. | + |
| Example 419. | + |
| Example 420. | + |
| Example 421. | + |
| Example 422. | + |
| Example 423. | + |
| Example 424. | ++ |
| Example 425. | |
| Example 426. | |
| Example 427. | |
| Example 428. | |
| Example 429. | + |
| Example 430. | + |
| Example 431. | |
| Example 432. | |
| Example 433. | + |
| Example 434. | + |
| Example 435. | ++ |
| Example 436. | + |
| Example 437. | + |
| Example 438. | + |
| Example 439. | + |
| Example 440. | + |
| Example 441. | ++ |
| Example 442. | ++ |
| Example 443. | ++ |
| Example 444. | ++ |
| Example 445. | ++ |
| Example 446. | ++ |
| Example 447. | ++ |
| Example 448. | ++ |
| Example 449. | + |
| Example 450. | + |
| Example 451. | + |
| Example 452. | + |
| Example 453. | + |
| Example 454. | + |
| Example 455. | + |
| Example 456. | + |
| Example 457. | + |
| Example 458. | + |
| Example 459. | + |
| Example 460. | + |
| Example 461. | |
| Example 462. | ++ |
| Example 463. | ++ |
| Example 464. | ++ |
| Example 465. | ++ |
| Example 466. | + |
| Example 467. | ++ |
| Example 468. | + |
| Example 469. | + |
| Example 470. | + |
| Example 471. | + |
| Example 472. | ++ |
| Example 473. | ++ |
| Example 474. | ++ |
| Example 475. | ++ |
| Example 476. | ++ |
| Example 477. | + |
| Example 478. | + |
| Example 479. | + |
| Example 480. | ++ |
| Example 481. | ++ |
| Example 482. | ++ |
| Example 483. | ++ |
| Example 484. | + |
| Example 485. | + |
| Example 486. | ++ |
| Example 487. | |
| Example 488. | |
| Example 489. | + |
| Example 490. | + |
| Example 491. | + |
| Example 492. | + |
| Example 493. | + |
| Example 494. | ++ |
| Example 495. | ++ |
| Example 496. | + |
| Example 497. | ++ |
| Example 498. | + |
| Example 499. | + |
| Example 500. | ++ |
| Example 501. | ++ |
| Example 502. | ++ |
| Example 503. | ++ |
| Example 504. | ++ |
| Example 505. | ++ |
| Example 506. | + |
| Example 507. | ++ |
| Example 508. | ++ |
| Example 509. | ++ |
| Example 510. | ++ |
| Example 511. | |
| Example 512. | |
| Example 513. | |
| Example 514. | |
| Example 515. | |
| Example 516. | ++ |
| Example 517. | ++ |
| Example 518. | ++ |
| Example 519. | ++ |
| Example 520. | ++ |
| Example 521. | ++ |
| Example 522. | + |
| Example 523. | + |
| Example 524. | ++ |
| Example 525. | + |
| Example 526. | + |
| Example 527. | ++ |
| Example 528. | + |
| Example 529. | ++ |
| Example 530. | |
| Example 531. | + |
| Example 532. | ++ |
| Example 533. | + |
| Example 534. | ++ |
| Example 535. | ++ |
| Example 536. | + |
| Example 537. | + |
| Example 538. | ++ |
| Example 539. | ++ |
| Example 540. | |

Blank = Not Determined

Effect of SOS1 Inhibitor (Compound A) on In Vivo Tumor Cell Growth Alone and in Combination with KRAS^{G12C} Inhibitor MRTX1257

Objective: To evaluate the efficacy of the SOS1 inhibitor Compound A alone and in combination with a KRAS G12C inhibitor MRTX1257 following oral administration in the human non-small cell lung cancer (NSCLC) NCI-H358 xenograft model in nude mice.

Methods: The effect of a SOS1 inhibitor of the present invention (Compound A) on tumor cell growth in vivo was evaluated in the NSCLC NCI-H358 xenograft model using female balb/c athymic nude mice (6-8 weeks old). Mice were implanted with H358 tumor cells in 50% matrigel (5^{e6} cells/mouse) subcutaneously in the flank. Once tumors reached an average size of ˜200 mm³, mice were randomized to treatment groups and administration of test article or vehicle (2% HPMC, 0.1% tween in 50 mM sodium citrate buffer pH 4). Body weight and tumor volume (using digital calipers) were measured twice a week until study endpoints. Compounds were administered by oral gavage daily.

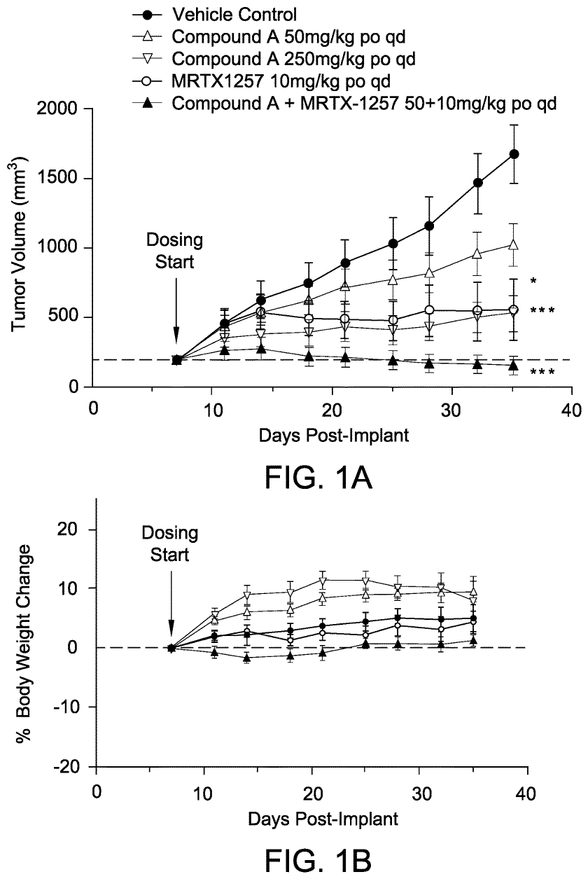

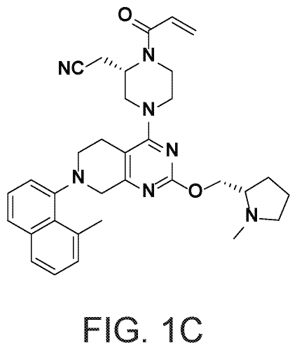

Results: FIG. 1A shows the efficacy of repeated daily dosing of Compound A at 50 and 250 mg/kg po (tumor growth inhibition, TGI=44 and 78% respectively) and MRTX1257 at 10 mg/kg (76%). Compound A at 250 mg/kg and MRTX1257 as a single agent caused significant tumor growth inhibition compared to the vehicle control, ***p≤0.001 and *p≤0.05 respectively, as assessed by an ordinary one-way ANOVA of tumor volumes along with multiple comparisons via a post-hoc Tukey's test in Graphpad Prism Software. Note for the H358 model, MRTX1257 at 50 mg/kg achieved regressions, thus a sub-optimal dose was used to observe combination effects with Compound A.

When dosed in combination, Compound A at 50 mg/kg with MRTX1257 at 10 mg/kg produced average regressions of 21%. At the end of study, 7/10 mice in the combination group achieved tumor regressions >10% reduction from baseline.

All treatments were well-tolerated for the duration of the study as evaluated by body weight (FIG. 1B). FIG. 1C shows the structure of MRTX1257. MRTX1257 is commercially available (CAS No. 2206736-04-9).

Conclusion: Compound A exhibited statistically significant and dose-dependent efficacy in the NCI-H358 non-small cell lung cancer xenograft model following oral administration at 50 mg/kg and 250 mg/kg daily. MRTX1257 also exhibited efficacy in this model at a sub-optimal dose of 10 mg/kg daily. Compound A as a single agent and in combination with MRTX1257 was well tolerated and the combination regimen resulted in 7/10 tumor regressions at the end of study.

### EQUIVALENTS

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

1. A compound having the structure of Formula (I), 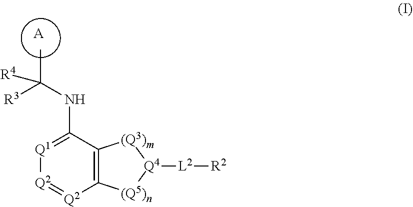
or a pharmaceutically acceptable salt, solvate, stereoisomer, prodrug, or tautomer thereof, wherein:
Q¹ is CH or N;
Q⁴ is CH, C, or N;
each Q² is independently C—R¹ or N, wherein one Q² is N and the other Q² is C—R¹;
each Q³ and Q⁵ are independently C(R^{QC})₂, NR^{QN}, CO, 0, S, or SO₂, wherein each R^{QC} is independently H, F, Cl, Br, or 6-10 membered aryl, and wherein each R^{QN} is independently H, C₁₋₆ alkyl, or 6-10 membered aryl;
wherein at least one of Q¹, Q², Q², Q³, Q⁴, and Q⁵ is N, NR^{QN}, O, or SO₂;
m is 0, 1, 2, or 3;
n is 0, 1, 2, or 3;
wherein when m is 0, then n is not 0;
R¹ is selected from the group consisting of H, C₁₋₆ alkyl, halogen, —CONHR^{1a}, —NHR^{1a}, —OR^{1a}, cyclopropyl, azetidinyl, and —CN; wherein each C₁₋₆ alkyl and azetidinyl is optionally substituted with halogen, R^{1a}, —NHR^{1a}, or —OR^{1a}; wherein R^{1a} is H, C₁₋₆ alkyl, cyclopropyl, 3-6 membered heterocyclyl, or C₁₋₆ haloalkyl;
L² is selected from the group consisting of a bond, —C(O)—, —C(O)O—, —C(O)NH(CH₂)ₒ—, S(O)₂—, 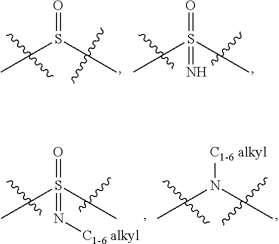
—C(O)(CH₂)ₚ—, —(CH₂)ₚ—, and —O—; wherein o is 0, 1, or 2; and wherein p is a number from 1 to 6;
R² is selected from the group consisting of H, C₁₋₆ alkyl, —NR^{2b}R^{2c}, —OR^{2a}, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl are independently optionally substituted with C₁₋₆ alkyl, C₁₋₆ haloalkyl, C₁₋₆ hydroxyalkyl, C₁₋₆ methoxyalkyl, —OH, —OR^{2a}, oxo, ═N, halogen, —C(O)R^{2a}, —C(O)OR^{2a}, —C(O)NR^{2b}R^{2c}, —SO₂R^{2a}, —CN, —NR^{2b}R^{2c}, 3-6 membered cycloalkyl, 3-7 membered heterocyclyl, 6-10 membered aryl, or 5-10 membered heteroaryl;
wherein R^{2a} is H, C₁₋₆ alkyl, C₁₋₆ haloalkyl, 3-7 membered heterocyclyl, or —(CH₂)ᵣOCH₃, wherein r is 1, 2, or 3;
wherein R^{2b} is H or C₁₋₆ alkyl;
wherein R^{2c} is H or C₁₋₆ alkyl;
R³ and R⁴ are independently H or C₁₋₆ alkyl optionally substituted with halo or —OH; wherein at least one of R³ and R⁴ is H or wherein R³ and R⁴ together with the atom to which they are attached combine to form a 3-6 membered cycloalkyl; and
A is an optionally substituted 6-membered aryl or an optionally substituted 5-6 membered heteroaryl;
with the proviso that when 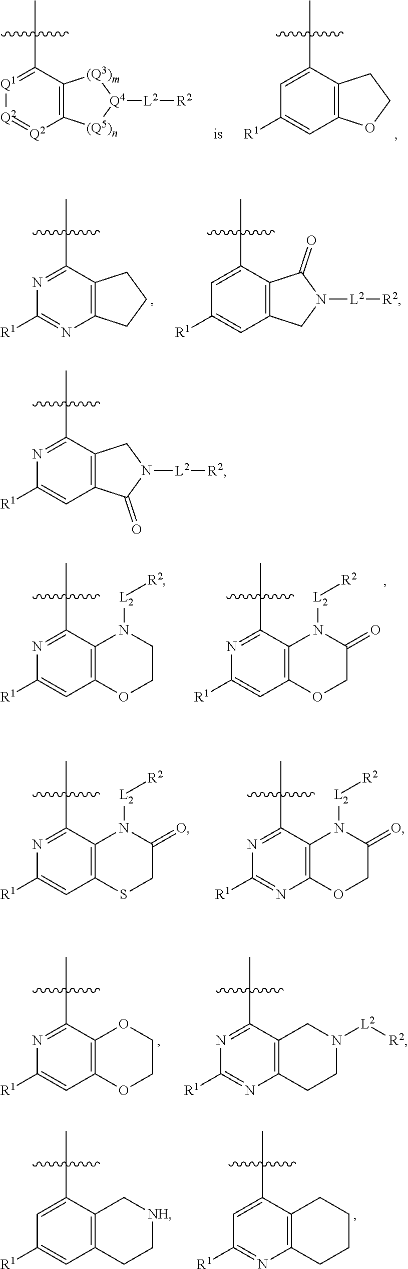 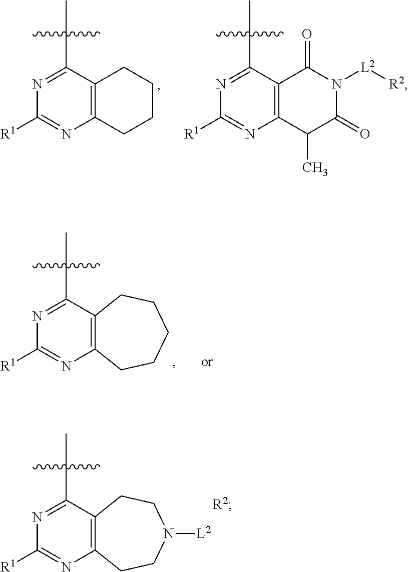
then R¹ is not H.

2. The compound of claim 1 having the structure of Formula (I-a), 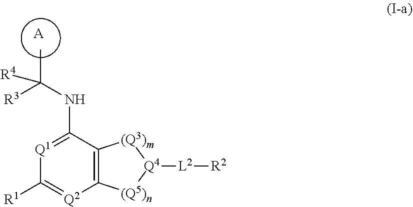
or a pharmaceutically acceptable salt, solvate, stereoisomer, prodrug, or tautomer thereof, wherein:
Q¹, Q³, Q⁴, Q⁵, m, n and A are as defined in claim 1;
Q² is CH or N;
wherein at least one of Q¹, Q², Q², Q³, Q⁴, and Q⁵ is N, NR^{QN}, O, or SO₂;
R¹ is selected from the group consisting of H, halogen, C₁₋₆ alkyl, cyclopropyl, —CN, and —OR^{1a}; wherein R^{1a} is H or C₁₋₆ alkyl;
L² is selected from the group consisting of a bond, —C(O)—, —C(O)O—, —C(O)NH(CH₂)ₒ—, —S(O)₂—, —C(O)(CH₂)ₚ—, —(CH₂)ₚ—, or —O—; wherein o is 0, 1, or 2; and wherein p is a number from 1 to 6;
R² is selected from the group consisting of H, —(CH₂)_{q}CH₃, 3-14 membered cycloalkyl, 3-14 membered cycloalkenyl, 3-14 membered heterocyclyl, 6-10 membered aryl, and 5-10 membered heteroaryl; wherein q is a number from 1 to 5; wherein each 3-14 membered cycloalkyl, 3-14 membered cycloalkenyl, 3-14 membered heterocyclyl, 6-10 membered aryl, or 5-10 membered heteroaryl is optionally substituted with C₁₋₆ alkyl, —OH, halogen, —C(O)R^{2a}, or —C(O)NR^{2b}R^{2c}; wherein R^{2a} is C₁₋₆ alkyl or —(CH₂)ᵣOCH₃, wherein r is 1, 2, or 3; wherein R^{2b} is H or C₁₋₆ alkyl; and wherein R^{2e} is H or C₁₋₆ alkyl; and
R³ and R⁴ are independently H or C₁₋₆ alkyl; wherein at least one of R³ and R⁴ is not H; or
R³ and R⁴ together with the atom to which they are attached combine to form a 3-6 membered cycloalkyl.

3. The compound of claim 1, or a pharmaceutically acceptable salt, solvate, stereoisomer, prodrug, or tautomer thereof, wherein A is an optionally substituted 6-membered aryl.

4. The compound of claim 1, or a pharmaceutically acceptable salt, solvate, stereoisomer, prodrug, or tautomer thereof, wherein A is an optionally substituted 5-6 membered heteroaryl.

5. The compound of claim 1 having the structure of Formula (V), 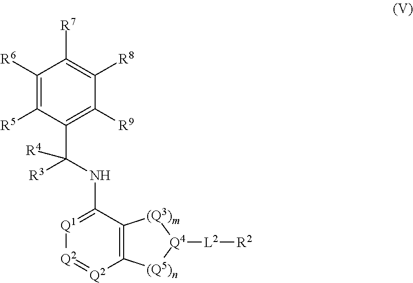
or a pharmaceutically acceptable salt, solvate, stereoisomer, prodrug, or tautomer thereof, wherein:
L², Q¹, Q², Q³, Q⁴, Q⁵, m, n, R¹, R², R³ and R⁴ are as defined in claim 1;
R⁵, R⁶, R⁷, R⁸, and R⁹ are independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OH, halogen, —NO₂, —CN, —NR¹¹R¹², —SR¹⁰, —S(O)₂NR¹¹R¹², S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, halogen, —NO₂, oxo, —CN, —R¹⁰, —OR¹⁰, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, 3-8 membered cycloalkyl, 3-14 membered heterocyclyl, 6-10 membered aryl, or 5-10 membered heteroaryl, or any two adjacent R⁵, R⁶, R⁷, R, and R⁹ forms a 3-14 membered fused ring;
R¹⁰, R¹¹, and R¹² are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR¹³, —SR¹³, halogen, —NR¹³R¹⁴, —NO₂, and —CN; and
R¹³ and R¹⁴ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN.

6. The compound of claim 5 having the structure of Formula (V-a), 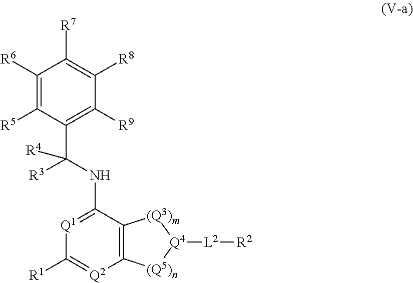
or a pharmaceutically acceptable salt, solvate, stereoisomer, prodrug, or tautomer thereof, wherein:
Q¹, Q³, Q⁴, Q⁵, m, n, R², R³, R⁴, R⁵, R⁶, R⁷, R⁸, R⁹, R¹⁰, R¹¹, R¹², R¹³ and R¹⁴ are as defined in claim 5;
Q² is CH or N;
wherein at least one of Q¹, Q², Q², Q³, Q⁴, and Q⁵ is N, NR^{QN}, O, or SO₂;
R¹ is selected from the group consisting of H, halogen, C₁₋₆ alkyl, cyclopropyl, —CN, and —OR^{1a}; wherein R^{1a} is H or C₁₋₆ alkyl; and
L² is selected from the group consisting of a bond, —C(O)—, —C(O)O—, —C(O)NH(CH₂)ₒ—, —S(O)₂—, —C(O)(CH₂)ₚ—, —(CH₂)ₚ—, or —O—; wherein o is 0, 1, or 2; and wherein p is a number from 1 to 6.

7. The compound of claim 1 having the structure of Formula (VI), 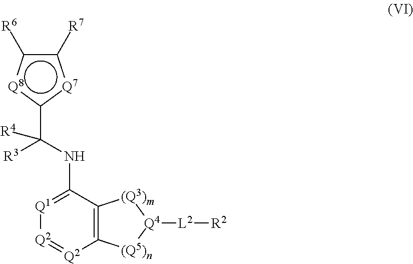
or a pharmaceutically acceptable salt, solvate, stereoisomer, prodrug, or tautomer thereof, wherein:
L², Q¹, Q², Q³, Q⁴, Q⁵, m, n, R¹, R², R³, and R⁴ are as defined in claim 1;
Q⁷ and Q⁸ are each independently CH, N, NH, 0, or S, provided at least one of Q⁷ and Q⁸ is N, NH, O, or S;
R⁶ and R⁷ are independently selected from the group consisting of H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OH, halogen, —NO₂, —CN, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, —C(O)R¹⁰, and —CO₂R¹⁰, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, halogen, —NO₂, oxo, —CN, —R¹⁰, —OR¹⁰, —NR¹¹R¹², —SR¹⁰, —S(O)₂NR¹¹R¹², —S(O)₂R¹⁰, —NR¹⁰S(O)₂NR¹¹R¹², —NR¹⁰S(O)₂R¹¹, —S(O)NR¹¹R¹², —S(O)R¹⁰, —NR¹⁰S(O)NR¹¹R¹², —NR¹⁰S(O)R¹¹, 3-8 membered cycloalkyl, 3-14 membered heterocyclyl, 6-10 membered aryl, or 5-10 membered heteroaryl,
R¹⁰, R¹¹, and R¹² are at each occurrence independently selected from H, D, C₁₋₆ alkyl, C₂₋₆ alkenyl, 4-8 membered cycloalkenyl, C₂₋₆ alkynyl, 3-8 membered cycloalkyl, 3-14 membered heterocyclyl, —OR¹³, —SR¹³, halogen, —NR¹³R¹⁴, —NO₂, and —CN; and
R¹³ and R¹⁴ are at each occurrence independently selected from H, D, C₁₋₆ alkyl, C₂₋₆ alkenyl, 4-8 membered cycloalkenyl, C₂₋₆ alkynyl, 3-8 membered cycloalkyl, and 3-14 membered heterocyclyl, wherein each C₁₋₆ alkyl, C₂₋₆ alkenyl, 4-8 membered cycloalkenyl, C₂₋₆ alkynyl, 3-8 membered cycloalkyl, and 3-14 membered heterocyclyl are independently optionally substituted with —OH, —SH, —NH₂, —NO₂, or —CN.

8. The compound of claim 7, or a pharmaceutically acceptable salt, solvate, stereoisomer, prodrug, or tautomer thereof, having the structure (VI-a), 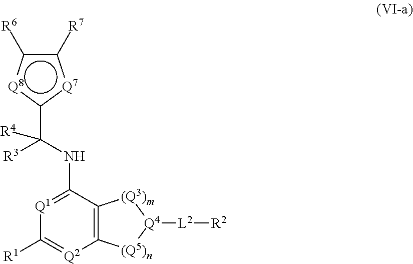
wherein L², Q¹, Q², Q³, Q⁴, Q⁵, Q⁷, Q⁸, R¹, R², R³, R⁴, R⁶, and R⁷ are as defined in claim 7.

9. The compound of claim 2, or a pharmaceutically acceptable salt, solvate, stereoisomer, prodrug, or tautomer thereof, wherein 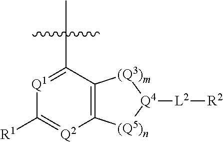
is selected from the group consisting of

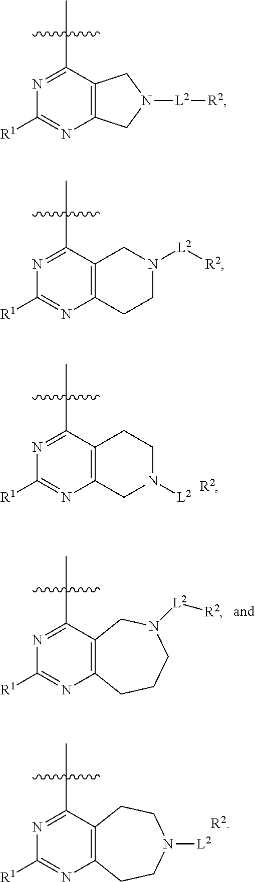

10. The compound of claim 2, or a pharmaceutically acceptable salt, solvate, stereoisomer, prodrug, or tautomer thereof, wherein 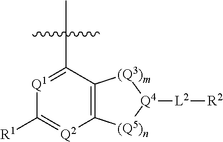
is selected from the group consisting of

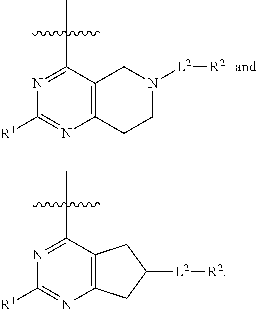

11. The compound of claim 1, or a pharmaceutically acceptable salt, solvate, stereoisomer, prodrug, or tautomer thereof, wherein R¹ is selected from the group consisting of H, C₁₋₆ alkyl, halogen, —NHR^{1a}, —OR^{1a}cyclopropyl, and —CN; wherein C₁₋₆ alkyl is optionally substituted with halogen, —NHR^{1a}, or —OR^{1a}; wherein R^{1a} is H, C₁₋₆ alkyl, 3-6 membered heterocyclyl, or C₁₋₆ haloalkyl.

12. The compound of claim 1, or a pharmaceutically acceptable salt, solvate, stereoisomer, prodrug, or tautomer thereof, wherein R¹ is H, halogen, C₁₋₆ alkyl, cyclopropyl, —CN, or —OR^{1a}; wherein Ria is H or C₁₋₆ alkyl.

13. The compound of claim 1, or a pharmaceutically acceptable salt, solvate, stereoisomer, prodrug, or tautomer thereof, wherein L² is selected from the group consisting of a bond, —C(O)—, —C(O)O—, —C(O)NH(CH₂)ₒ—, —S(O)₂—, —C(O)(CH₂)ₚ—, —(CH₂)ₚ—, and —O—; wherein o is 0, 1, or 2; and wherein p is a number from 1 to 6.

14. The compound of claim 1, or a pharmaceutically acceptable salt, solvate, stereoisomer, prodrug, or tautomer thereof,
wherein L² is selected from the group consisting of

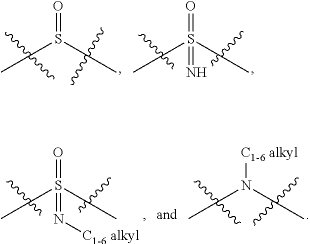

15. The compound of claim 1, or a pharmaceutically acceptable salt, solvate, stereoisomer, prodrug, or tautomer thereof, wherein R² is selected from the group consisting of H, C₁₋₆ alkyl, —NR^{2b}R^{2c}, —OR^{2a}, 3-14 membered cycloalkyl, 3-14 membered cycloalkenyl, 3-14 membered heterocyclyl, 6-10 membered aryl, and 5-10 membered heteroaryl; wherein each C₁₋₆ alkyl, 3-14 membered cycloalkyl, 3-14 membered cycloalkenyl, 3-14 membered heterocyclyl, 6-10 membered aryl, and 5-10 membered heteroaryl are independently optionally substituted with C₁₋₆ alkyl, —OH, —OR^{2a}, oxo, ═N, halogen, —C(O)R^{2a}, —C(O)OR^{2a}, —C(O)NR^{2b}R^{2c}, —CN, —NR^{2b}R^{2c}, 3-6 membered cycloalkyl, 3-7 membered heterocyclyl, 6-10 membered aryl, or 5-10 membered heteroaryl.

16. The compound of claim 1, or a pharmaceutically acceptable salt, solvate, stereoisomer, prodrug, or tautomer thereof, wherein R² is 3-14 membered heterocyclyl, wherein the 3-14 membered heterocyclyl is optionally substituted with C₁₋₆ alkyl optionally substituted with halogen or —OR²a, —OH, —OR^{2a}, oxo, ═N, halogen, —C(O)R^{2a}, —C(O)OR^{2a}, —C(O)NR^{2b}R^{2c}, —SO₂R^{2a}, —CN, —NR^{2b}R^{2c}, 3-6 membered cycloalkyl, 3-7 membered heterocyclyl, 6-10 membered aryl, or 5-10 membered heteroaryl; wherein R^{2a} is H, C₁₋₆ alkyl, C₁₋₆ haloalkyl, 3-7 membered heterocyclyl, or —(CH₂)ᵣOCH₃, wherein r is 1, 2, or 3; wherein R^{2b} is H or C₁₋₆ alkyl; and wherein R², is H or C₁₋₆ alkyl.

17. The compound of claim 2, or a pharmaceutically acceptable salt, solvate, stereoisomer, prodrug, or tautomer thereof, wherein R³ is H and R⁴ is —CH₃ and the compound is of the following formula: 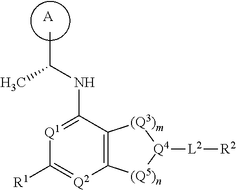
wherein A, L², Q¹, Q², Q³, Q⁴, Q⁵, R¹, R², m and n are as defined in claim 2.

18. The compound of claim 5, or a pharmaceutically acceptable salt, solvate, stereoisomer, prodrug, or tautomer thereof, wherein R³ is H and R⁴ is —CH₃ and the compound is of the following formula: 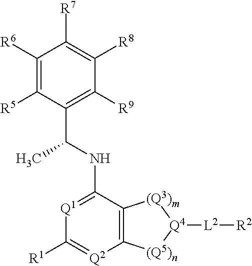
wherein L², Q¹, Q², Q³, Q⁴, Q⁵, R¹, R², R⁴, R⁵, R⁶, R⁷, R⁸, R⁹, m and n are as defined in claim 5.

19. The compound of claim 1, or a pharmaceutically acceptable salt, solvate, hydrate, tautomer, or stereoisomer thereof, selected from the group consisting of compounds of Collection 1.

20. The compound of claim 1, or a pharmaceutically acceptable salt, solvate, hydrate, tautomer, or stereoisomer thereof, selected from the group consisting of compounds of Collection 2.

21. The compound of claim 1, or a pharmaceutically acceptable salt, solvate, hydrate, tautomer, or stereoisomer thereof, selected from the group consisting of compounds of Collection 3.

22. The compound of claim 1, or a pharmaceutically acceptable salt, solvate, hydrate, tautomer, or stereoisomer thereof, selected from the group consisting of compounds of Table A.

23. A pharmaceutical composition comprising a compound of claim 1, or a pharmaceutically acceptable salt, solvate, hydrate, tautomer, or stereoisomer thereof, and a pharmaceutically acceptable carrier.

24. A method of inhibiting SOS1 in a subject, comprising administering to the subject a compound of claim 1, or a pharmaceutically acceptable salt, solvate, hydrate, tautomer, or stereoisomer thereof.

25. A method of inhibiting the interaction of SOS1 and a RAS-family protein in a cell or inhibiting the interaction of SOS1 and RAC1 in a cell, comprising administering to the cell a compound of claim 1, or a pharmaceutically acceptable salt, solvate, hydrate, tautomer, or stereoisomer thereof.

26. A method of treating or preventing a disease, wherein treating or preventing the disease is characterized by inhibition of the interaction of SOS1 and a RAS-family protein or by inhibition of the interaction of SOS1 and RAC1, the method comprising administering to a subject in need thereof an effective amount of a compound of claim 1, or a pharmaceutically acceptable salt, solvate, hydrate, tautomer, or stereoisomer isomer thereof.

27. A method of treating or preventing cancer in a subject in need thereof, comprising administering to the subject an effective amount of a compound of claim 1, or a pharmaceutically acceptable salt, solvate, hydrate, tautomer, or stereoisomer isomer thereof.

28. The method of claim 27, wherein the cancer is selected from the group consisting of pancreatic cancer, lung cancer, colorectal cancer, hematological cancer, cholangiocarcinoma, multiple myeloma, melanoma, uterine cancer, endometrial cancer, thyroid cancer, acute myeloid leukemia, bladder cancer, urothelial cancer, gastric cancer, cervical cancer, head and neck squamous cell carcinoma, diffuse large B cell lymphoma, esophageal cancer, chronic lymphocytic leukemia, hepatocellular cancer, breast cancer, ovarian cancer, prostate cancer, glioblastoma, renal cancer and sarcomas.

29. The method of claim 28, wherein the disease is a RASopathy.

30. The method of claim 29, wherein the RASopathy is selected from the group consisting of Neurofibromatosis type 1 (NF1), Noonan Syndrome (NS), Noonan Syndrome with Multiple Lentigines (NSML), Capillary Malformation-Arteriovenous Malformation Syndrome (CM-AVM), Costello Syndrome (CS), Cardio-Facio-Cutaneous Syndrome (CFC), Legius Syndrome, and Hereditary gingival fibromatosis.
